(19) United States
(12) Patent Application Publication
(10) Pub. No.: US 2022/0177587 A1
(43) Pub. Date: Jun. 9, 2022

(54) METHODS AND USES OF VARIANT CD80 FUSION PROTEINS AND RELATED CONSTRUCTS

(71) Applicant: Alpine Immune Sciences, Inc., Seattle, WA (US)

(72) Inventors: Ryan SWANSON, Seattle, WA (US); Mark F. MAURER, Seattle, WA (US); Stanford L. PENG, Seattle, WA (US); Jing YANG, Seattle, WA (US); Kristine M. SWIDEREK, Seattle, WA (US)

(73) Assignee: Alpine Immune Sciences, Inc., Seattle, WA (US)

(21) Appl. No.: 17/275,646

(22) PCT Filed: Sep. 19, 2019
(86) PCT No.: PCT/US2019/052022

§ 371 Date: Mar. 11, 2021

Related U.S. Application Data
(60) Provisional application No. 62/818,058, filed on Mar. 13, 2019.
(60) Provisional application No. 62/733,623, filed on Sep. 19, 2018.
(60) Provisional application No. 62/733,625, filed on Sep. 19, 2018.

(51) Int. Cl. C07K 16/28; A61K 31/282; C07K 14/705; C12N 15/62; A61P 35/00; A61K 45/06

(52) U.S. Cl. CPC C07K 16/2818; A61K 31/282; C07K 14/70532; A61K 2039/505; A61P 35/00; A61K 45/06; C12N 15/62

(57) ABSTRACT

Provided herein are variant CD80 polypeptides, immunomodulatory proteins comprising variant CD80 polypeptides, and nucleic acids encoding such proteins. The immunomodulatory proteins provide therapeutic utility for a variety of oncological conditions. Compositions and methods for making and using such proteins are provided.

### CROSS-REFERENCE TO RELATED APPLICATIONS

This application claims priority from U.S. provisional application No. 62/733,625, filed Sep. 19, 2018, entitled “METHODS AND USES OF VARIANT CD80 FUSION PROTEINS AND RELATED CONSTRUCTS”; U.S. provisional application No. 62/733,623, filed Sep. 19, 2018, entitled “VARIANT CD80 FUSION PROTEINS AND RELATED COMPOSITIONS AND METHODS”; and U.S. provisional application No. 62/818,058, filed Mar. 13, 2019, entitled “METHODS AND USES OF VARIANT CD80 FUSION PROTEINS AND RELATED CONSTRUCTS”, the contents of each of which are incorporated by reference in their entirety.

### INCORPORATION BY REFERENCE OF SEQUENCE LISTING

The present application is being filed along with a Sequence Listing in electronic format. The Sequence Listing is provided as a file entitled 761612003040SeqList.txt, created Sep. 18, 2019, which is 2,178,803 bytes in size. The information in the electronic format of the Sequence Listing is incorporated by reference in its entirety.

### FIELD

The present disclosure relates to therapeutic compositions for modulating immune response in the treatment of cancer and methods of using the same. In some aspects, the present disclosure relates to particular variants of CD80 that exhibit altered binding, such as binding affinity or selectivity, for a cognate binding partner, such as increased affinity for CD28, PD-L1, and/or CTLA-4.

### BACKGROUND

Modulation of the immune response by intervening in the processes that occur in the immunological synapse (IS) formed by and between antigen-presenting cells (APCs) or target cells and lymphocytes is of increasing medical interest. Mechanistically, cell surface proteins in the IS can involve the coordinated and often simultaneous interaction of multiple protein targets with a single protein to which they bind. IS interactions occur in close association with the junction of two cells, and a single protein in this structure can interact with both a protein on the same cell (cis) as well as a protein on the associated cell (trans), likely at the same time. Although therapeutics are known that can modulate the IS, improved therapeutics are needed. Provided are immunomodulatory proteins, including soluble proteins or transmembrane immunomodulatory proteins capable of being expressed on cells, that meet such needs.

### SUMMARY

Provided herein are methods of treating a cancer in a subject. In some embodiments, the method includes administering to a subject having a cancer a variant CD80 fusion protein that specifically binds to PD-L1, said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide; and administering to the subject a therapeutically effective amount of an anti-cancer agent.

In some embodiments, the anti-cancer agent is an immune checkpoint inhibitor or a chemotherapeutic agent. In some embodiments, the anti-cancer agent is a chemotherapeutic agent that is a platinum-based chemotherapeutic agent. In some embodiments, the chemotherapeutic agent is oxilaplatin. In some embodiments, the anti-cancer agent is an immune checkpoint inhibitor of CTLA-4, optionally wherein the checkpoint inhibitor is an anti-CTLA-4 antibody or an antigen-binding fragment thereof. In some embodiments, the immune checkpoint inhibitor is ipilimumab or tremelimumab, or an antigen binding fragment thereof. In some embodiments, the anti-cancer agent is an immune checkpoint inhibitor of PD-1 (PD-1 inhibitor), optionally wherein the PD-1 inhibitor is an anti-PD-1 antibody or antigen binding fragment thereof.

Provided herein are methods of treating a cancer in a subject. In some embodiments, the method includes administering to a subject having a cancer a variant CD80 fusion protein that specifically binds to PD-L1, said variant CD80 fusion protein comprising a variant CD80 extracellular domain or a portion thereof comprising an IgV domain or a specific binding fragment thereof and a multimerization domain, wherein the variant CD80 extracellular domain or the portion thereof contains one or more amino acid modifications at one or more positions in the sequence of amino acids of the extracellular domain or a portion thereof of an unmodified CD80 polypeptide; and administering to the subject a therapeutically effective amount of a PD-1 inhibitor, wherein the PD-1 inhibitor disrupts the interaction between Programmed Death-1 (PD-1) and a ligand thereof.

In some embodiments, the ligand is Programmed Death Ligand-1 (PD-L1) or PD-L2. In some embodiments, the PD-1 inhibitor specifically binds to PD-1. In some embodiment, the PD-1 inhibitor does not compete with the variant CD80 fusion protein for binding to PD-L1. In some embodiments, the PD-1 inhibitor is a peptide, protein, antibody or antigen-binding fragment thereof, or a small molecule. In some embodiments, the PD-1 inhibitor is an antibody or antigen-binding fragment thereof that specifically binds to PD-1. In some examples, the antibody or antigen-binding portion is selected from nivolumab, pembrolizumab, MEDI0680 (AMP514), PDR001, cemiplimab (REGN2810), pidilizumab (CT011), or an antigen-binding portion thereof.

In some embodiments, the PD-1 inhibitor contains the extracellular domain of PD-L2 or a portion thereof that binds to PD-1, and an Fc region. In some embodiments, the PD-1 inhibitor is AMP-224.

In some embodiments, the initiation of the administration of the PD-1 inhibitor is carried out concurrently or sequentially with the initiation of the administration of the variant CD80 fusion protein. In some examples, the initiation of the administration of the PD-1 inhibitor is after the initiation of the administration of the variant CD80 fusion protein. In some embodiments, the initiation of the administration of the anti-PD-1 antibody is after the administration of the last dose of a therapeutically effective amount of the variant CD80 fusion protein. In some of any such embodiments, the variant CD80 fusion protein is administered in a therapeutically effective amount as a single dose or in six or fewer multiple doses.

Provided herein are methods of treating a cancer in a subject. In some embodiments, the method includes administering to a subject having a cancer a therapeutically effective amount of a variant CD80 fusion protein, said variant CD80 fusion protein comprising a variant CD80 extracellular domain or a portion thereof comprising an IgV domain or a specific binding fragment thereof and a multimerization domain, wherein the variant CD80 extracellular domain or the portion thereof contains one or more amino acid modifications at one or more positions in the sequence of amino acids of the extracellular domain or a portion thereof of an unmodified CD80 polypeptide, wherein the therapeutically effective amount of the variant CD80 fusion protein is administered as a single dose or in six or fewer multiple doses.

In some embodiments, the variant CD80 fusion protein, e.g. variant CD80 Fc fusion, is administered parenterally. In some embodiments, the variant CD80 fusion protein, e.g. variant CD80 Fc fusion, is administered subcutaneously. In some embodiments, the variant CD80 Fc fusion protein is administered intravenously. In some embodiments, the administration is by injection in which the injection is a bolus injection.

In embodiments of any of the provided methods, the therapeutically effective amount that is administered is between about 0.5 mg/kg and about 40 mg/kg, about 0.5 mg/kg and about 30 mg/kg, about 0.5 mg/kg and about 20 mg/kg, about 0.5 mg/kg and about 18 mg/kg, about 0.5 mg/kg and about 12 mg/kg, about 0.5 mg/kg and about 10 mg/kg, about 0.5 mg/kg and about 6 mg/kg, about 0.5 mg/kg and about 3 mg/kg, about 1 mg/kg and about 40 mg/kg, about 1 mg/kg and about 30 mg/kg, about 1 mg/kg and about 20 mg/kg, about 1 mg/kg and about 18 mg/kg, about 1 mg/kg and about 12 mg/kg, about 1 mg/kg and about 10 mg/kg, about 1 mg/kg and about 6 mg/kg, about 1 mg/kg and about 3 mg/kg, about 3 mg/kg and about 40 mg/kg, about 3 mg/kg and about 30 mg/kg, about 3 mg/kg and about 20 mg/kg, about 3 mg/kg and about 18 mg/kg, about 3 mg/kg and about 12 mg/kg, about 3 mg/kg and about 10 mg/kg, about 3 mg/kg and about 6 mg/kg, about 6 mg/kg and about 40 mg/kg, about 6 mg/kg and about 30 mg/kg, about 6 mg/kg and about 20 mg/kg, about 6 mg/kg and about 18 mg/kg, about 6 mg/kg and about 12 mg/kg, about 6 mg/kg and about 10 mg/kg, about 10 mg/kg and about 40 mg/kg, about 10 mg/kg and about 30 mg/kg, about 10 mg/kg and about 20 mg/kg, about 10 mg/kg and about 18 mg/kg, about 10 mg/kg and about 12 mg/kg, about 12 mg/kg and about 40 mg/kg, about 12 mg/kg and about 30 mg/kg, about 12 mg/kg and about 20 mg/kg, about 12 mg/kg and about 18 mg/kg, about 18 mg/kg and about 40 mg/kg, about 18 mg/kg and about 30 mg/kg, about 18 mg/kg and about 20 mg/kg, about 20 mg/kg and about 40 mg/kg, about 20 mg/kg and about 30 mg/kg or about 30 mg/kg and about 40 mg/kg, each inclusive. In some embodiments, the therapeutically effective amount is between about 3.0 mg/kg and 18 mg/kg, inclusive. In some embodiments, the therapeutically effective amount is between about 6 mg/kg and about 20 mg/kg, inclusive.

In some of any such embodiments, the therapeutically effective amount is between about 1 mg/kg and about 10 mg/kg, inclusive. In some embodiments, the therapeutically effective amount is between about 2.0 mg/kg and about 6.0 mg/kg, inclusive. In some embodiments, the variant CD80 fusion protein, e.g. variant CD80 Fc fusion, is administered intratumorally.

Provided herein are methods of treating a cancer in a subject. In some embodiments, the method includes intratumorally administering to a subject having a cancer a therapeutically effective amount of a variant CD80 fusion protein, said variant CD80 fusion protein comprising a variant CD80 extracellular domain or a portion thereof comprising an IgV domain or a specific binding fragment thereof and a multimerization domain, wherein the variant CD80 extracellular domain or the portion thereof contains one or more amino acid modifications at one or more positions in the sequence of amino acids of the extracellular domain or a portion thereof of an unmodified CD80 polypeptide. In some of any such embodiments, the variant CD80 fusion protein is administered in a therapeutically effective amount as a single dose or in six or fewer multiple doses. In some embodiments, the therapeutically effective amount is between about 0.1 mg/kg and about 1 mg/kg, inclusive. In some examples, the therapeutically effective amount is between about 0.2 mg/kg and about 0.6 mg/kg. In some embodiments, the therapeutically effective amount is administered in a single dose.

In some of any such provided embodiments, the therapeutically effective amount is administered in six or fewer multiple doses and the six or fewer multiple doses is two doses, three doses, four doses, five doses or six doses. In some embodiment, the therapeutically effective amount is administered in four doses. In some embodiments, the therapeutically effective amount is administered in three doses. In some examples, the therapeutically effective amount is administered in two doses.

In some embodiments, each dose of the multiple dose is administered weekly, every two weeks, every three weeks or every four weeks. In some embodiments, each of the six or fewer multiple doses is administered weekly, every two weeks, every three weeks, or every four weeks. In some aspects, the interval between each multiple dose is about a week.

In some of any of the provided embodiments, the single dose or each of the multiple doses, such as each of the six of fewer multiple doses, is administered in an amount between about 0.5 mg/kg and about 10 mg/kg once every week (Q1W).

Provided herein are methods of treating a cancer in a subject, the method including administering to a subject having a cancer a variant CD80 fusion protein in an amount of between about 1.0 mg/kg to 10 mg/kg, inclusive, once every week (Q1W), said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide.

In some embodiments the amount of the variant CD80 fusion protein administered Q1W is between about 1 mg/kg and about 3 mg/kg.

In some of any of the provided embodiments, the single dose or each of the multiple doses, such as each of the six or fewer multiple doses, is administered in an amount between about 1.0 mg/kg and about 40 mg/kg once every three weeks (Q3W).

Provided herein are methods of treating a cancer in a subject, the method including administering to a subject having a cancer a variant CD80 fusion protein in an amount of between about 1.0 mg/kg to 40 mg/kg, inclusive, once every three weeks (Q3W), said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide.

In some embodiments, the amount of the variant CD80 fusion protein administered Q3W is between about 3.0 mg/kg and about 10 mg/kg Q3W.

In some of any of the provided embodiments, the variant CD80 fusion protein is administered parenterally, optionally subcutaneously. In some embodiments, the variant CD80 fusion protein is administered by injection that is a bolus injection.

In some of any of the provided embodiments, the administration is for more than one week. In some examples, the therapeutically effective amount is administered in a time period of no more than six weeks. In some embodiments, the therapeutically effective amount is administered in a time period of no more than four weeks or about four weeks. In some embodiment, each multiple dose is an equal amount.

In some of any such embodiments, the method includes prior to the administering, selecting a subject for treatment that has a tumor comprising cells surface positive for PD-L1 or CD28 and/or surface negative for a cell surface ligand selected from CD80 or CD86. In some embodiments, a subject is selected for treatment that has a tumor comprising cells that are surface positive for PD-L1. In some embodiments, a subject is selected for treatment that has a tumor comprising cells that are surface positive for CD28. In some embodiments, a subject is selected for treatment that has a tumor comprising cells that are surface negative for CD80. In some embodiments, a subject is selected for treatment that has a tumor comprising cells that are surface negative for CD86. In particular aspects, such cells are tumor cells. In particular aspects, such cells are tumor infiltrating immune cells, such as tumor infiltrating T lymphocytes.

Provided herein are methods of treating a cancer in a subject, the method including administering a variant CD80 fusion protein to a subject selected as having a tumor containing cells surface negative for a cell surface ligand selected from CD80 or CD86, and/or surface positive for CD28, wherein the variant CD80 fusion protein contains a variant CD80 extracellular domain or a portion thereof comprising an IgV domain or a specific binding fragment thereof and a multimerization domain, said variant CD80 extracellular domain or the portion thereof comprising one or more amino acid modifications at one or more positions in the sequence of amino acids of the extracellular domain or a portion thereof of an unmodified CD80 polypeptide.

In some embodiments, the cells surface negative for CD80 or CD86 contain tumor cells or antigen presenting cells. In some embodiments, the cells surface positive for CD28 contain tumor infiltrating T lymphocytes. In some examples, the subject has further been selected as having a tumor comprising cells surface positive for PD-L1. In some embodiments, the cells surface positive for PD-L1 are tumor cells or tumor infiltrating immune cells, optionally tumor infiltrating T lymphocytes.

In some embodiments, the method includes determining an immunoscore based on the presence or density of tumor infiltrating T lymphocytes in the tumor of the subject. In some embodiments, the subject is selected for treatment if the immunoscore is low. In some of any such embodiments, a subject is selected by immunohistochemistry (IHC) using a reagent that specifically binds to the at least one binding partner.

In some embodiments, the variant CD80 fusion protein exhibits increased binding to at least one binding partner selected from among CD28, PD-L1 and CTLA-4 compared to a fusion protein comprising the extracellular domain of the unmodified CD80 for the at least one binding partner. In some examples, the variant CD80 fusion protein exhibits increased binding to PD-L1 compared to a fusion protein comprising the extracellular domain of the unmodified CD80 for the binding partner. In some embodiments, the variant CD80 fusion protein further exhibits increased binding to at least one binding partner selected from among CD28 and CTLA-4 compared to a fusion protein comprising the extracellular domain of the unmodified CD80 for the at least one binding partner. In some of any such embodiments, the binding, such as affinity, is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 80-fold, 100-fold, 150-fold, 200-fold, 250-fold, 300-fold, 400-fold, or 450-fold compared to the binding, such as affinity, of the unmodified CD80 for the ectodomain of the binding partner.

In some embodiments, the variant CD80 fusion protein exhibits increased binding to at least one binding partner selected from among CD28, PD-L1 and CTLA-4 compared to a fusion protein comprising the extracellular domain or portion thereof of the unmodified CD80 for the at least one binding partner. In some examples, the variant CD80 fusion protein exhibits increased binding to PD-L1 compared to a fusion protein comprising the extracellular domain or portion thereof of the unmodified CD80 for the binding partner PD-L1. In some embodiments, the variant CD80 fusion protein further exhibits increased binding to at least one binding partner selected from among CD28 and CTLA-4 compared to a fusion protein comprising the extracellular domain or portion thereof of the unmodified CD80 for the at least one binding partner. In some of any such embodiments, the binding affinity is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 80-fold, 100-fold, 150-fold, 200-fold, 250-fold, 300-fold, 400-fold, or 450-fold compared to binding affinity of the unmodified CD80 for the ectodomain of the binding partner.

In some of any of the provided embodiments, the one or more amino acid modifications are amino acid substitutions. In some examples, the one or more amino acid modifications contain one or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof. In some embodiments, the one or more amino acid modifications contain two or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

In some examples, the one or more amino acid modifications contain amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2. In some embodiments, the one or more amino acid modifications contain amino acid substitutions E35D/M47L/V68M, E35D/M47V/V68M or E35D/M47I/L70M.

In some of any such embodiments, the one or more amino acid modifications contain amino acid substitutions E35D/M47V/N48K/V68M/K89N.

In some of any such embodiments, the one or more amino acid modifications contain amino acid substitutions H18Y/A26E/E35D/M47L/V68M/A71G/D90G.

In some of any such embodiments, the one or more amino acid modifications contain amino acid substitutions E35D/D46E/M47V/V68M/D90G/K93E.

In some of any such embodiments, the one or more amino acid modifications contain amino acid substitutions E35D/D46V/M47L/V68M/L85Q/E88D.

In some of any such embodiments, the unmodified CD80 is a human CD80.

In some of any such embodiments, the extracellular domain or portion thereof of the unmodified CD80 contains (i) the sequence of amino acids set forth in SEQ ID NO:2, (ii) a sequence of amino acids that has at least 95% sequence identity to SEQ ID NO:2; or (iii) is a portion of (i) or (ii) comprising an IgV domain or a specific binding fragment thereof.

In some embodiments, the extracellular domain or portion thereof of the unmodified CD80 is an extracellular domain portion that is or contains the IgV domain or a specific binding fragment thereof. In some embodiments, the extracellular domain portion of the unmodified CD80 contains the IgV domain but does not contain the IgC domain or a portion of the IgC domain. In some embodiments, the extracellular domain portion of the unmodified CD80 is set forth as the sequence of amino acids 35-135 of SEQ ID NO:2 (SEQ ID NO:76) or 35-141 of SEQ ID NO:2 (SEQ ID NO:150). In some embodiments, the variant CD80 extracellular domain or portion thereof is an extracellular domain portion that does not contain the IgC domain or a portion of the IgC domain.

In some embodiments, the variant CD80 extracellular domain contains the sequence of amino acids 35-135 of SEQ ID NO:2 (SEQ ID NO:76) or 35-141 of SEQ ID NO:2 (SEQ ID NO:150) in which is contained the one or more amino acid substitutions. In some embodiments, the variant CD80 extracellular domain is the sequence of amino acids 35-135 of SEQ ID NO:2 (SEQ ID NO:76) or 35-141 of SEQ ID NO:2 (SEQ ID NO:150) in which is contained the one or more amino acid substitutions. In some embodiments, the variant CD80 extracellular domain 1, 2, 3, 4, 5, 6, 7, 8, 9, 10, 11, 12, 13, 14, 15, 16, 17, 18, 19 or 20 amino acid modifications, optionally wherein the amino acid modifications are amino acid substitutions.

In some of any such embodiments, the variant CD80 extracellular domain contains no more than 1, 2, 3, 4, 5, 6, 7, 8, 9, 10, 11, 12 or 13 amino acid modifications. In some of any such embodiments, the variant CD80 extracellular domain or the portion thereof contains no more than 1, 2, 3, 4, 5, 6, 7, 8, 9, 10, 11, 12 or 13 amino acid modifications. In some such embodiments, the amino acid modifications are amino acid substitutions. In some embodiments, the amino acid sequence of the variant CD80 extracellular domain has at least or at least about 85%, 86%, 87%, 88%, 89%, 90%, 91%, 92%, 93%, 94%, 95%, 96%, 97%, 98%, 99% or more sequence identity to the sequence of amino acids 35-135 of SEQ ID NO:2 (SEQ ID NO:76) or 35-141 of SEQ ID NO:2 (SEQ ID NO:150).

In some of any such embodiments, the multimerization domain is an Fc region. In some embodiments, the Fc region is of an immunoglobulin G1 (IgG1) or an immunoglobulin G2 (IgG2) protein. In some embodiments, the Fc region exhibits one or more effector functions. In some embodiments, the Fc region is a variant Fc region comprising one or more amino acid substitutions in a wildtype Fc region, said variant Fc region exhibiting one or more effector function that is reduced compared to the wildtype Fc region, such as reduced compared to the wildtype human Fc is of human IgG1.

In some embodiments, the Fc region contains the amino acid substitution N297G, wherein the residue is numbered according to the EU index of Kabat. In some embodiments, the Fc region contains the amino acid substitutions R292C/N297G/V302C, wherein the residue is numbered according to the EU index of Kabat. In some embodiments, the Fc region contains the amino acid substitutions L234A/L235E/G237A, wherein the residue is numbered according to the EU index of Kabat. In some embodiments, the Fc region further contains the amino acid substitution C220S, wherein the residues are numbered according to the EU index of Kabat. In some embodiments, the Fc region contains K447del, wherein the residue is numbered according to the EU index of Kabat.

In some of any such embodiments, the variant CD80 fusion protein antagonizes the activity of CTLA-4. In some embodiments, the variant CD80 fusion protein blocks the PD-1/PD-L1 interaction. In some embodiments, the variant CD80 fusion proteins binds to CD28 and mediates CD28 agonism. In some embodiments, the CD28 agonism is PD-L1 dependent. In some embodiments, the subject is a human.

Provided herein are kits containing: a variant CD80 fusion protein that specifically binds to PD-L1, said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide; and an anticancer agent.

In some embodiments, the anti-cancer agent is an immune checkpoint inhibitor or a chemotherapeutic agent. In some embodiments, the anti-cancer agent is a chemotherapeutic agent that is a platinum-based chemotherapeutic agent. In some embodiments, the chemotherapeutic agent is oxilaplatin. In some embodiments, the anti-cancer agent is an immune checkpoint inhibitor of CTLA-4, optionally wherein the checkpoint inhibitor is an anti-CTLA-4 antibody or an antigen-binding fragment thereof. In some embodiments, the immune checkpoint inhibitor is ipilimumab or tremelimumab, or an antigen binding fragment thereof. In some embodiments, the anti-cancer agent is an immune checkpoint inhibitor of PD-1 (PD-1 inhibitor), optionally wherein the PD-1 inhibitor is an anti-PD-1 antibody or antigen binding fragment thereof.

Provided herein are kits containing: a variant CD80 fusion protein that specifically binds to PD-L1, said variant CD80 fusion protein comprising a variant CD80 extracellular domain or a portion thereof comprising an IgV domain or a specific binding fragment thereof and a multimerization domain, wherein the variant CD80 extracellular domain or the portion thereof contains one or more amino acid modifications at one or more positions in the sequence of amino acids of the extracellular domain or a portion thereof of an unmodified CD80 polypeptide; and a PD-1 inhibitor, wherein the PD-1 inhibitor disrupts the interaction between Programmed Death-1 (PD-1) and a ligand thereof.

In some embodiments, the ligand is Programmed Death Ligand-1 (PD-L1) or PD-L2. In some embodiments, the PD-1 inhibitor specifically binds to PD-1. In some embodiments, the PD-1 inhibitor does not compete with the variant CD80 fusion protein for binding to PD-L1. In some embodiments, the PD-1 inhibitor is a peptide, protein, antibody or antigen-binding fragment thereof, or a small molecule. In some embodiments, the PD-1 inhibitor is an antibody or antigen-binding fragment thereof that specifically binds to PD-1.

In some of any such embodiments, the antibody or antigen-binding portion is selected from nivolumab, pembrolizumab, MEDI0680 (AMP514), PDR001, cemiplimab (REGN2810), pidilizumab (CT011), or an antigen-binding portion thereof.

In some embodiments, the PD-1 inhibitor contains the extracellular domain of PD-L2 or a portion thereof that binds to PD-1, and an Fc region. In some embodiments, the PD-1 inhibitor is AMP-224. In some embodiments, the variant CD80 fusion protein exhibits increased binding to at least one binding partner selected from among CD28, PD-L1 and CTLA-4 compared to a fusion protein comprising the extracellular domain or portion thereof of the unmodified CD80 for the at least one binding partner. In some embodiments, the variant CD80 fusion protein exhibits increased binding to PD-L1 compared to a fusion protein comprising the extracellular domain or portion thereof of the unmodified CD80 for PD-1.

In some embodiments, the variant CD80 fusion protein further exhibits increased binding to at least one binding partner selected from among CD28 and CTLA-4 compared to a fusion protein comprising the extracellular domain of the unmodified CD80 for the at least one binding partner. In some embodiments, the variant CD80 fusion protein exhibits increased binding to at least one binding partner selected from among CD28 and CTLA-4 compared to a fusion protein comprising the extracellular domain or portion thereof of the unmodified CD80 for the at least one binding partner. In some embodiments, the binding, such as affinity, is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 80-fold, 100-fold, 150-fold, 200-fold, 250-fold, 300-fold, 400-fold, or 450-fold compared to binding affinity of the unmodified CD80 for the ectodomain of the binding partner.

In some of any such embodiments, the one or more amino acid modifications are amino acid substitutions. In some embodiments, the one or more amino acid modifications contain one or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof. In some embodiments, the one or more amino acid modifications contain two or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

In some of any of the provided embodiments, the one or more amino acid modifications contain amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2. In some embodiments, the one or more amino acid modifications contain amino acid substitutions E35D/M47L/V68M, E35D/M47V/V68M or E35D/M47I/L70M. In some embodiments, the one or more amino acid modifications contain amino acid substitutions E35D/M47V/N48K/V68M/K89N, H18Y/A26E/E35D/M47L/V68M/A71G/D90G, E35D/D46E/M47V/V68M/D90G/K93E or E35D/D46V/M47L/V68M/L85Q/E88D.

In some of any such embodiments, the unmodified CD80 is a human CD80. In some embodiments, the extracellular domain or portion thereof of the unmodified CD80 contains (i) the sequence of amino acids set forth in SEQ ID NO:2, (ii) a sequence of amino acids that has at least 95% sequence identity to SEQ ID NO:2; or (iii) is a portion of (i) or (ii) comprising an IgV domain or a specific binding fragment thereof.

In some embodiments, the extracellular domain or portion thereof of the unmodified CD80 is an extracellular domain portion that is or contains the IgV domain or a specific binding fragment thereof. In some embodiments, the extracellular domain portion of the unmodified CD80 contains the IgV domain but does not contain the IgC domain or a portion of the IgC domain.

In some embodiments, the extracellular domain portion of the unmodified CD80 is set forth as the sequence of amino acids 35-135 of SEQ ID NO:2 (SEQ ID NO:76) or 35-141 of SEQ ID NO:2 (SEQ ID NO:150). In some embodiments, the variant CD80 extracellular domain or portion thereof is an extracellular domain portion that does not contain the IgC domain or a portion of the IgC domain.

In some embodiments, the variant CD80 extracellular domain contains the sequence of amino acids 35-135 of SEQ ID NO:2 (SEQ ID NO:76) or 35-141 of SEQ ID NO:2 (SEQ ID NO:150) in which is contained the one or more amino acid substitutions. In some embodiments, the variant CD80 extracellular domain is the sequence of amino acids 35-135 of SEQ ID NO:2 (SEQ ID NO:76) or 35-141 of SEQ ID NO:2 (SEQ ID NO:150) in which is contained the one or more amino acid substitutions. In some embodiments, the variant CD80 extracellular domain 1, 2, 3, 4, 5, 6, 7, 8, 9, 10, 11, 12, 13, 14, 15, 16, 17, 18, 19 or 20 amino acid modifications, optionally wherein the amino acid modifications are amino acid substitutions.

In some embodiments, the variant CD80 extracellular domain contains no more than 1, 2, 3, 4, 5, 6, 7, 8, 9, 10, 11, 12 or 13 amino acid modifications. In some such embodiments, the amino acid modifications are amino acid substitutions. In some embodiments, the variant CD80 extracellular domain has at least or at least about 85%, 86%, 87%, 88%, 89%, 90%, 91%, 92%, 93%, 94%, 95%, 96%, 97%, 98%, 99% or more sequence identity to the sequence of amino acids 35-135 of SEQ ID NO:2 (SEQ ID NO:76) or 35-141 of SEQ ID NO:2 (SEQ ID NO:150).

In some of any such provided embodiments, the multimerization domain is an Fc region. In some embodiments, the Fc region is of an immunoglobulin G1 (IgG1) or an immunoglobulin G2 (IgG2) protein. In some embodiments, the Fc region exhibits one or more effector functions. In some embodiments, the Fc region is a variant Fc region containing one or more amino acid substitutions in a wildtype Fc region, said variant Fc region exhibiting one or more effector function that is reduced compared to the wildtype Fc region, optionally wherein the wildtype human Fc is of human IgG1.

Provided herein are articles of manufacture containing the kit of any of such embodiments and instructions for use. In some embodiments, the instructions provide information for administration of the variant CD80 fusion protein, such as variant CD80 Fc fusion protein, or PD-1 inhibitor in accord with any of the provided methods.

Provided herein is a multivalent CD80 polypeptide containing two copies of a fusion protein containing: at least two variant CD80 extracellular domains or a portion thereof comprising an IgV domain or a specific binding fragment thereof (vCD80), wherein the vCD80 contains one or more amino acid modifications at one or more positions in the sequence of amino acids of the extracellular domain or a portion thereof of an unmodified CD80 polypeptide and an Fc polypeptide.

In some embodiments, the polypeptide is tetravalent. In some embodiments, the fusion protein contains the structure: (vCD80)-Linker-Fc-Linker-(vCD80). In some embodiments, the fusion protein contains the structure: (vCD80)-Linker-(vCD80)-Linker-Fc.

In some embodiments, the vCD80 exhibits increased binding to at least one binding partner selected from among CD28, PD-L1 and CTLA-4 compared to a vCD80 comprising the extracellular domain or portion thereof of the unmodified CD80 for the at least one binding partner. In some embodiments, the vCD80 exhibits increased binding to PD-L1 compared to the extracellular domain or portion thereof of the unmodified CD80 for PD-L1. In some embodiments, the vCD80 exhibits increased binding to at least one binding partner selected from among CD28, PD-L1 and CTLA-4 compared to a vCD80 comprising the extracellular domain of the unmodified CD80 for the at least one binding partner. In some embodiments, the binding, such as affinity, is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 80-fold, 100-fold, 150-fold, 200-fold, 250-fold, 300-fold, 400-fold, or 450-fold compared to binding affinity of the unmodified CD80 for the ectodomain of the binding partner.

In some embodiments, the one or more amino acid modifications are amino acid substitutions. In some embodiments, the one or more amino acid modifications contain one or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

In some of any such embodiments, the one or more amino acid modifications are amino acid substitutions. In some embodiments, the one or more amino acid modifications contain one or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

In some embodiments, the one or more amino acid modifications contain two or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof. In some embodiments, the one or more amino acid modifications contains amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2.

In some embodiments, the one or more amino acid modifications contain amino acid substitutions E35D/M47L/V68M, E35D/M47V/V68M or E35D/M47I/L70M. In some embodiments, the one or more amino acid modifications contain amino acid substitutions E35D/M47V/N48K/V68M/K89N, H18Y/A26E/E35D/M47L/V68M/A71G/D90G, E35D/D46E/M47V/V68M/D90G/K93E or E35D/D46V/M47L/V68M/L85Q/E88D. In some embodiments, the unmodified CD80 is a human CD80.

In some embodiments, the extracellular domain or portion thereof of the unmodified CD80 contains (i) the sequence of amino acids set forth in SEQ ID NO:2, (ii) a sequence of amino acids that has at least 95% sequence identity to SEQ ID NO:2; or (iii) is a portion of (i) or (ii) comprising an IgV domain or a specific binding fragment thereof. In some examples, the extracellular domain or portion thereof of the unmodified CD80 is an extracellular domain portion that is or contains the IgV domain or a specific binding fragment thereof.

In some embodiments, the extracellular domain portion of the unmodified CD80 contains the IgV domain but does not contain the IgC domain or a portion of the IgC domain. In some embodiments, the extracellular domain portion of the unmodified CD80 is set forth as the sequence of amino acids 35-135 of SEQ ID NO:2 (SEQ ID NO:76) or 35-141 of SEQ ID NO:2 (SEQ ID NO:150). In some examples, the vCD80 is an extracellular domain portion that does not contain the IgC domain or a portion of the IgC domain.

In some of any such embodiments, the vCD80 contains the sequence of amino acids 35-135 of SEQ ID NO:2 (SEQ ID NO:76) or 35-141 of SEQ ID NO:2 (SEQ ID NO:150) in which is contained the one or more amino acid substitutions. In some embodiments, the vCD80 has the sequence of amino acids 35-135 of SEQ ID NO:2 (SEQ ID NO:76) or 35-141 of SEQ ID NO:2 (SEQ ID NO:150) in which is contained the one or more amino acid substitutions. In some embodiments, the vCD80 contains 1, 2, 3, 4, 5, 6, 7, 8, 9, 10, 11, 12, 13, 14, 15, 16, 17, 18, 19 or 20 amino acid modifications, optionally wherein the amino acid modifications are amino acid substitutions. In some embodiments, the vCD80 contains no more than 1, 2, 3, 4, 5, 6, 7, 8, 9, 10, 11, 12 or 13 amino acid modifications, optionally wherein the amino acid modifications are amino acid substitutions. In some embodiments, the vCD80 has at least or at least about 85%, 86%, 87%, 88%, 89%, 90%, 91%, 92%, 93%, 94%, 95%, 96%, 97%, 98%, 99% or more sequence identity to the sequence of amino acids 35-135 of SEQ ID NO:2 (SEQ ID NO:76) or 35-141 of SEQ ID NO:2 (SEQ ID NO:150).

In some embodiments, the multimerization domain is an Fc region. In some embodiments, the Fc region is of an immunoglobulin G1 (IgG1) or an immunoglobulin G2 (IgG2) protein. In some embodiments, the Fc region exhibits one or more effector functions. In some embodiments, the Fc region is a variant Fc region comprising one or more amino acid substitutions in a wildtype Fc region, said variant Fc region exhibiting one or more effector function that is reduced compared to the wildtype Fc region, optionally wherein the wildtype human Fc is of human IgG1.

In some of any such embodiments, each vCD80 is the same. In some embodiments, the linker is a flexible linker. In some embodiments, the linker is a peptide linker. In some embodiments, the linker is GSGGGGS (SEQ ID NO:1522) or 3× GGGGS (SEQ ID NO: 1504).

Provided herein is a nucleic acid molecule encoding the multivalent CD80 polypeptide of any of any such embodiments.

Provided herein is a nucleic acid molecule encoding the fusion protein of the multivalent CD80 polypeptide of any of any such embodiments.

Provided herein is a vector containing the nucleic acid of any of such embodiments. In some embodiments, the vector is an expression vector.

Provided herein is a host cell containing the nucleic acid or the vector of any of such embodiments.

Provided herein is a method of producing a multivalent CD80 polypeptide of any of such embodiments, the method including introducing the nucleic acid of any of such embodiments or the vector of any of such embodiments into a host cell under conditions to express the protein in the cell. In some embodiments, the method includes isolating or purifying the protein containing the multivalent CD80 polypeptide.

Provided herein is an engineered cell comprising the multivalent CD80 polypeptide of any of such embodiments. In some embodiments, the multivalent CD80 polypeptide comprises a fusion protein encoded by a nucleic acid molecule operably linked to a sequence encoding a secretory signal peptide. In some embodiments, the multivalent CD80 polypeptide is capable of being secreted from the engineered cell when expressed.

Provided herein is an engineered cell, comprising the nucleic acid molecule or a vector of any of such embodiments. In some embodiments, the nucleic acid molecule comprises a sequence encoding a secretory signal peptide operably linked to the sequence encoding the fusion protein. In some embodiments, the nucleic acid molecule encodes a fusion protein of a multivalent CD80 polypeptide, wherein the multivalent CD80 polypeptide is capable of being secreted from the engineered cell when expressed. In some embodiments, the signal peptide is a non native signal sequence. In some embodiments, the signal peptide is an IgG kappa signal peptide, an IL-2 signal peptide, a CD33 signal peptide or a VH signal peptide.

In some embodiments, the nucleic acid molecule further comprises at least one promoter operably linked to control expression of the fusion protein. In some embodiments, the promoter is a constitutively active promoter. In some embodiments, the promoter is an inducible promoter. In some embodiments, the promoter is responsive to an element responsive to T-cell activation signaling, optionally wherein the promoter comprises a binding site for NFAT or a binding site for NF-κB.

In some embodiments, the cell is an immune cell, optionally an antigen presenting cell (APC) or a lymphocyte. In some embodiments, the cell is a lymphocyte that is a T cell, a B cell or an NK cell, optionally wherein the lymphocyte is a T cell that is CD4+ or CD8+. In some embodiments, the cell is a primary cell obtained from a subject, optionally wherein the subject is a human subject.

In some embodiments, the cell further comprises a chimeric antigen receptor (CAR) or an engineered T cell receptor (TCR).

Provided herein is a pharmaceutical composition containing the multivalent CD80 polypeptide of any of such embodiments.

Provided herein is a pharmaceutical composition comprising the engineered cell of any of such embodiments.

Provided herein is a variant CD80 fusion protein comprising: (i) a variant extracellular domain comprising one or more amino acid substitutions at one or more positions in the sequence of amino acids set forth as amino acid residues 35-230 of a wildtype human CD80 extracellular domain corresponding to residues set forth in SEQ ID NO:1 and (ii) an Fc region that has effector activity, wherein the extracellular domain of the variant CD80 fusion protein specifically binds to the ectodomain of human CD28 and does not bind to the ectodomain of human PD-L1 or binds to the ectodomain of PD-L1 with a similar binding affinity as the extracellular domain of the wildtype human CD80 for the ectodomain of PD-L1.

In some embodiments, the extracellular domain of the variant CD80 fusion protein exhibits increased binding affinity to the ectodomain of human CTLA-4 compared to the binding affinity of the extracellular domain of wildtype CD80 for the ectodomain of human CTLA-4. In some embodiments, the extracellular domain of the variant CD80 fusion protein exhibits increased binding affinity to the ectodomain of human CD28 compared to the binding affinity of the extracellular domain of wildtype CD80 for the ectodomain of human CD28.

In some embodiments, the wildtype human CD80 extracellular domain has the sequence of amino acids set forth in SEQ ID NO:2 or a sequence that has at least 95%, 96%, 97%, 98%, 99% or more sequence identity to SEQ ID NO:2. In some embodiments, the one or more amino acid substitutions comprise one or more amino acid substitutions selected from L70Q, K89R, D90G, D90K, A91G, F92Y, K93R, I118V, T120S or T130A, with reference to numbering set forth in SEQ ID NO:2, or a conservative amino acid substitution thereof. In some embodiments, the one or more amino acid substitutions comprise amino acid modifications L70Q/K89R, L70Q/D90G, L70Q/D90K, L70Q/A91G, L70Q/F92Y, L70Q/K93R, L70Q/I118V, L70Q/T120S, L70Q/T130A, K89R/D90G, K89R/D90K, K89R/A91G, K89R/F92Y, K89R/K93R, K89R/I118V, K89R/T120S, K89R/T130A, D90G/A91G, D90G/F92Y, D90G/K93R, D90G/I118V, D90G/T120S, D90G/T130A, D90K/A91G, D90K/F92Y, D90K/K93R, D90K/I118V, D90K/T120S, D90K/T130A, F92Y/K93R, F92Y/I118V, F92Y/T120S, F92Y/T130A, K93R/I118V, K93R/T120S, K93R/T130A, I118V/T120S, I118V/T130A or T120S/T130A.

In some embodiments, the one or more amino acid substitutions comprise one or more amino acid substitutions selected from substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof. In some embodiments, the one or more amino acid substitutions comprises amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2.

In some embodiments, the Fc region is of an immunoglobulin G1 (IgG1).

Provided herein is a nucleic acid molecule encoding the variant CD80 fusion protein of any of such embodiments.

Provided herein is a vector comprising the nucleic acid of any of such embodiments, optionally wherein the vector is an expression vector.

Provided herein is a host cell comprising the nucleic acid or the vector of any of such embodiments.

Provided herein is a method of producing a variant CD80 fusion protein of any of such embodiments, comprising introducing the nucleic acid or the vector of any of such embodiments into a host cell under conditions to express the protein in the cell, optionally wherein the method further comprises isolating or purifying the protein comprising the variant CD80 fusion protein.

Provided herein is a pharmaceutical composition comprising the variant CD80 fusion protein of any of such embodiments.

In some embodiments, the pharmaceutical composition contains a pharmaceutically acceptable excipient. In some embodiments, the pharmaceutical composition is sterile.

Provided herein is an article of manufacture containing the pharmaceutical composition of any of such embodiments in a container' in some embodiments, optionally the container is a vial. In some embodiments, the container is sealed.

Provided herein is a method of modulating an immune response in a subject, including administering the pharmaceutical composition of any of such embodiments to a subject or the multivalent CD80 polypeptide of any of such embodiments to a subject. In some embodiments, the method includes modeling the immune response treats a disease or condition in the subject.

Provided herein is a method of modulating an immune response in a subject, comprising administering the multivalent CD80 polypeptide of any of such embodiments to a subject.

Provided herein is a method of modulating an immune response in a subject, comprising administering the engineered cell of any of such embodiments to a subject. In some embodiments, the engineered cell is autologous to the subject. In some embodiments, modulating the immune response treats a disease or condition in the subject. In some embodiments, the disease or condition is a tumor or cancer.

Provided herein is a method of treating a cancer in a subject, including administering the pharmaceutical composition of any of such embodiments to a subject or the multivalent CD80 polypeptide of any of any of such embodiments to a subject.

Provided herein is a method of treating a cancer in a subject, comprising administering the pharmaceutical composition, the multivalent CD80 polypeptide, or the engineered cell of any of such embodiments to a subject.

Provided herein is a variant CD80 fusion protein containing: a variant extracellular domain comprising one or more amino acid substitutions at one or more positions in the sequence of amino acids set forth as amino acid residues 35-230 of a wildtype human CD80 extracellular domain and an Fc region that has effector activity, wherein the extracellular domain of the variant CD80 fusion protein specifically binds to the ectodomain of human CD28 and does not bind to the ectodomain of human PD-L1 or binds to the ectodomain of PD-L1 with a similar binding affinity as the extracellular domain of the wildtype human CD80 for the ectodomain of PD-L1.

In some embodiments, the extracellular domain of the variant CD80 fusion protein exhibits increased binding affinity to the ectodomain of human CTLA-4 compared to the binding affinity of the extracellular domain of wildtype CD80 for the ectodomain of human CTLA-4. In some of any such embodiments, the extracellular domain of the variant CD80 fusion protein exhibits increased binding affinity to the ectodomain of human CD28 compared to the binding affinity of the extracellular domain of wildtype CD80 for the ectodomain of human CD28. In some embodiments, the affinity is increased about or greater than 1.2-fold, 1.5-fold, 2-fold, 3-fold, 4-fold, 5-fold, 6-fold, 7-fold, 8-fold, 9-fold, 10-fold or more.

In some embodiments, the variant CD80 fusion protein increases immunological activity in a mixed lymphocyte reaction, optionally wherein the increased immunological activity includes increased production of IFN-gamma or interleukin 2 in the mixed lymphocyte reaction. In some embodiments, the variant CD80 fusion protein increases immunological activity as assessed in a T cell reporter assay incubated with antigen presenting cells. In some embodiments, the variant CD80 fusion protein increases CD28-mediated costimulation of T lymphocytes. In some aspects, the increase is by about or greater than 1.2-fold, 1.5-fold, 2-fold, 3-fold, 4-fold, 5-fold, 6-fold, 7-fold, 8-fold, 9-fold, 10-fold or more.

In some of any such embodiments, the wildtype human CD80 extracellular domain has the sequence of amino acids set forth in SEQ ID NO:2 or a sequence that has at least 95%, 96%, 97%, 98%, 99% or more sequence identity to SEQ ID NO:2. In some embodiments, the wildtype human CD80 extracellular domain has the sequence of amino acids set forth in SEQ ID NO:2.

In some embodiments the one or more amino acid substitutions contain one or more amino acid substitutions selected from L70Q, K89R, D90G, D90K, A91G, F92Y, K93R, I118V, T120S or T130A, with reference to numbering set forth in SEQ ID NO:2, or a conservative amino acid substitution thereof. In some examples, the one or more amino acid substitutions contain two or more amino acid substitutions selected from L70Q, K89R, D90G, D90K, A91G, F92Y, K93R, I118V, T120S or T130A, with reference to numbering set forth in SEQ ID NO:2, or a conservative amino acid substitution thereof.

In some embodiments, the one or more amino acid substitutions contain amino acid modifications L70Q/K89R, L70Q/D90G, L70Q/D90K, L70Q/A91G, L70Q/F92Y, L70Q/K93R, L70Q/I118V, L70Q/T120S, L70Q/T130A, K89R/D90G, K89R/D90K, K89R/A91G, K89R/F92Y, K89R/K93R, K89R/I118V, K89R/T120S, K89R/T130A, D90G/A91G, D90G/F92Y, D90G/K93R, D90G/I118V, D90G/T120S, D90G/T130A, D90K/A91G, D90K/F92Y, D90K/K93R, D90K/I118V, D90K/T120S, D90K/T130A, F92Y/K93R, F92Y/I118V, F92Y/T120S, F92Y/T130A, K93R/I118V, K93R/T120S, K93R/T130A, I118V/T120S, I118V/T130A or T120S/T130A.

In some embodiments, the one or more amino acid substitutions contain amino acid substitutions A91G/I118V/T120S/T130A. In some examples, the one or more amino acid substitutions contain amino acid substitutions S21P/L70Q/D90G/I118V/T120S/T130A. In some embodiments, the one or more amino acid substitutions contain amino acid substitutions E88D/K89R/D90K/A91G/F92Y/K93R. In some examples, the one or more amino acid substitutions contain one or more amino acid substitutions selected from substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

In some of any such embodiments, the one or more amino acid substitutions contains amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2. In some embodiments, the one or more amino acid modifications contain amino acid substitutions E35D/M47L/V68M, E35D/M47V/V68M or E35D/M47I/L70M.

In some embodiments, the one or more amino acid modifications contain amino acid substitutions E35D/M47V/N48K/V68M/K89N, H18Y/A26E/E35D/M47L/V68M/A71G/D90G, E35D/D46E/M47V/V68M/D90G/K93E or E35D/D46V/M47L/V68M/L85Q/E88D. In some aspects, the variant CD80 extracellular domain has 1, 2, 3, 4, 5, 6, 7, 8, 9, 10, 11, 12, 13, 14, 15, 16, 17, 18, 19 or 20 amino acid substitutions. In some examples, the variant CD80 extracellular domain contains no more than 1, 2, 3, 4, 5, 6, 7, 8, 9, 10, 11, 12 or 13 amino acid substitutions. In some embodiments, the variant CD80 extracellular domain has at least or at least about 85%, 86%, 87%, 88%, 89%, 90%, 91%, 92%, 93%, 94%, 95%, 96%, 97%, 98%, 99% or more sequence identity to the sequence of amino acids set forth in SEQ ID NO:2.

In some of any such embodiments, the Fc region is of an immunoglobulin G1 (IgG1). In some examples, the Fc region contains the amino acid substitution C220S, wherein the residues are numbered according to the EU index of Kabat. In some embodiments, the Fc region contains K447del, wherein the residue is numbered according to the EU index of Kabat.

In some aspects, the Fc region as the sequence of amino acids set forth in SEQ ID NO: 1502, 1510, 1517 or 1527. In some embodiments, the one or more effector function is selected from among antibody dependent cellular cytotoxicity (ADCC), complement dependent cytotoxicity, programmed cell death and cellular phagocytosis. In some of any such embodiments, the variant CD80 fusion protein is a dimer.

Provided herein is a nucleic acid molecule encoding the variant CD80 fusion protein of any of such embodiments.

Provided herein is a vector containing the nucleic acid of any of such embodiments. In some embodiments, the vector is an expression vector.

Provided herein is a host cell containing the nucleic acid of any of such embodiments or the vector of any of such embodiments.

Provided herein is a method of producing a variant CD80 fusion protein of any of such embodiments, including introducing the nucleic acid or the vector of any of such embodiments into a host cell under conditions to express the protein in the cell. In some embodiments, the method further includes isolating or purifying the protein containing the variant CD80 fusion protein.

Provided herein is a pharmaceutical composition containing the variant CD80 fusion protein of any of such embodiments. In some embodiments, the pharmaceutical composition contains a pharmaceutically acceptable excipient. In some embodiments, the pharmaceutical composition is sterile.

Provided herein is an article of manufacture containing the pharmaceutical composition of any of such embodiments in a container, optionally wherein the container is a vial. In some embodiments, the container is sealed.

Provided herein is a method of modulating an immune response in a subject, including administering the pharmaceutical composition of any of such embodiments to a subject or the variant CD80 fusion protein of any of any of such embodiments to a subject. In some aspects, modulating the immune response treats a disease or condition in the subject. In some examples, the disease or condition is a tumor or cancer.

Provided herein is a method of treating a cancer in a subject, including administering the pharmaceutical composition of any of such embodiments to a subject or the variant CD80 fusion protein of any of such embodiments to a subject.

### BRIEF DESCRIPTION OF THE DRAWINGS

FIG. 1A depicts an exemplary schematic of the activity of a CD80 variant IgSF domain (vIgD), conjugated to an Fc, in which the CD80-Fc blocks PD-1/PD-L1 inhibitory activity. As shown, binding of the CD80 vIgD-Fc to PD-L1, thereby antagonizing binding of PD-L1 to its cognate binding partners PD-1, and blocking PD-1 inhibitory signaling, reducing the TCR signaling threshold, and promoting T cell activation.

FIG. 1B depicts an exemplary schematic of the activity of a variant IgSF domain (vIgD)—conjugated to an Fc in which the CD80-Fc effects PD-L1-dependent CD28 agonist activity. As shown, binding of the CD80-Fc to PD-L1, expressed on the surface of a tumor cell, can prevent the association of the PD-L1 on the tumor cell and the inhibitory PD-1 receptor, expressed on the surface of a T cell. In addition, the CD80-Fc is available to bind the costimulatory CD28 receptor on the surfaces of a T cell, thereby localizing the T cell to the tumor while promoting T cell activation via CD28 costimulation of TCR signal.

FIG. 2A depicts an exemplary schematic of the activity of a variant IgSF domain (vIgD) fused to an Fc (vIgD-Fc) in which the vIgD is a variant of an IgSF domain of CD80. As shown, a soluble vIgD of CD80 interacts with its cognate binding partners to block interaction of CD80 with CTLA-4, thereby blocking the CTLA-4 inhibitory receptor, and, in some cases, allowing the T cell to differentiate into an effector phenotype.

FIG. 2B depicts an exemplary schematic of the activity of a CD80 variant IgSF domain (vIgD), conjugated to an Fc, in which the CD80-Fc blocks CTLA-4 inhibitory activity. As shown, binding of the CD80 vIgD-Fc to CTLA-4, expressed on the surface of T cells (e.g., T_{reg} and T_{eff} cells), thereby antagonizing binding of CTLA-4 to its cognate binding partners CD80 (B7-1) and CD86 (B7-2), indicated as B7, and blocking CTLA-4 inhibitory signaling, reducing the TCR signaling threshold, and promoting T cell activation.

FIG. 3 depicts various exemplary configurations of a multivalent molecule containing a first CD80 vIgD and a second CD80 vIgD. As shown, the first CD80 vIgD and second CD80 vIgD are independently linked, directly or indirectly, to the N- or C-terminus of an Fc region. For generating a homodimeric Fc molecule, the Fc region is one that is capable of forming a homodimer with a matched Fc region by co-expression of the individual Fc regions in a cell. For generating a heterodimeric Fc molecule, the individual Fc regions contain mutations (e.g., “knob-into-hole” mutations in the CH3 domain), such that formation of the heterodimer is favored compared to homodimers when the individual Fc regions are co-expressed in a cell. In some embodiments, the first CD80 vIgD and second CD80 vIgD are the same or are different. The configurations shown result in proteins that are bivalent, tetravalent, or hexavalent for one or more of its cognate binding partners.

FIG. 4 depicts binding of exemplary CD80 IgV-Fc variants to cell surface-expressed PD-L1, CD28 and CTL44 ligands.

FIG. 5 depicts dose-dependent PD-L1-dependent CD28 costimulation in a Jurkat/IL-2 reporter line induced by exemplary CD80 IgV-Fc variants.

FIG. 6 depicts human primary T cell cytokine production following PD-L1-dependent costimulation induced by exemplary CD80 IgV-Fc variants.

FIG. 7 depicts the ability of exemplary CD80 IgV-Fc candidates to bind PD-L1 and block fluorescently conjugated PD-1 binding.

FIG. 8 depicts the PD-1/PD-L1 interaction and subsequent functional activity antagonistic activity of exemplary variant CD80-Fc variants.

FIG. 9 depicts the in vivo anti-tumor activity of exemplary variant CD80 polypeptides fused to wild-type IgG1 Fc (WT Fc) or inert IgG1 Fc (inert Fc).

FIG. 10 depicts the median (left panel) and mean (right panel) tumor volumes in a mouse model following treatment with an Inert Fc control; 50 μg, 100 μg, or 500 μg of an exemplary variant CD80 IgV-Fc (inert); or 100 μg anti-PD-L1 antibody (durvalumab). All animals were treated on days 8, 10, and 12 (left three arrows on each of the left and right panels). On days 26, 28, and 31, only animals that initially received the Inert Fc control then also received 100 μg of the exemplary variant CD80 IgV-Fc (right three arrows on each of the left and right panels).

FIG. 11 depicts concentration of IFNγ in hPD-L1MC38 tumor lysates following in vivo treatment with 50 μg, 100 μg, and 500 μg of an exemplary variant CD80 IgV-Fc (inert) and 100 μg anti-PD-L1 antibody (durvalumab).

FIG. 12 depicts the median (left panel) and mean (right panel) tumor volumes in a mouse model following treatment with multiple exemplary CD80 IgV-Fc (inert) variants and anti-PD-L1 antibody (durvalumab).

FIG. 13 depicts the median (left panel) and mean (right panel) tumor volumes in mice, designated tumor-free post-treatment with exemplary CD80 IgV-Fc (inert) variants and anti-PD-L1 antibody (durvalumab), following re-challenge with huPD-L1/MC38 tumor cells.

FIG. 14 depicts detection of bound negative control Fc, CD80 variant-Fc, and anti-PD-L1 antibody by flow cytometry on single cell suspensions of live CD45 negative (CD45 neg.; CD45-) tumor cells.

FIG. 15 depicts the median (top panel) and mean (bottom panel) tumor volumes in a mouse model following treatment with an exemplary variant CD80 IgV-Fc (inert) and anti-PD-L1 antibody (durvalumab).

FIGS. 16A and 16B depict percentage of CD8 cells detected by flow cytometry in the tumor draining lymph node (FIG. 16A) and tumor (FIG. 16B) of mice treated with negative control Fc, CD80 variant-Fc, and anti-PD-L1 antibody.

FIG. 16C represents the percentage of anti-human Fc detected reagents on CD45 negative tumors treated in vivo with negative control Fc, CD80 IgV-Fc, and human anti-PD-L1 antibody.

FIG. 17 depicts specific in vitro cytotoxic activity of CD80 IgV-Fc variants against huPD-L1 transduced MC38 tumor cells but not non-transduced parental MC38, demonstrating huPDL1 specific killing.

FIGS. 18 and 19 depict the binding of CD80 IgV-Fc variants to primary human T cells (FIG. 18) and primary human monocytes (FIG. 19).

FIG. 20 depicts CD80 IgV-Fc variant antagonism of PD-L1-mediated SHP-2 recruitment to PD-1 using an enzyme complementation assay.

FIG. 21 depicts CD80 IgV-Fc variant antagonism of CD80/CTLA-4 binding.

FIG. 22A shows median tumor volumes from assessment of anti-tumor activity of an exemplary tested variant CD80 IgV-Fc alone and in combination with anti-mouse PD-1 monoclonal antibody in a syngeneic mouse melanoma model. FIG. 22B shows anti-tumor activity measured by TGI.

FIG. 23 shows IL-2 production in an assessment of T cell response with a combination of an exemplary tested variant CD80 IgV-Fc alone and in combination with an anti-PD-1 antibody.

FIG. 24A shows median tumor volumes from assessment of anti-tumor activity from treatment with IP (intraperitoneal) or IT (intratumoral injections) with variant CD80 IgV-Fc.

FIG. 24B shows percent of cells detected using huIgG among CD45-negative cell subset from mice treated IP (intraperitoneal) or IT (intratumoral injections) with variant CD80 IgV-Fc.*, **, **** p<0.05, 0.001, 0.0001, respectively, vs Fc control group by 1-way ANOVA.

FIG. 24C shows percent of cells detected using huIgG among PD-L1+CD45− cell subset from mice treated IP (intraperitoneal) or IT (intratumoral injections) with variant CD80 IgV-Fc. *, **** p<0.05, 0.0001, respectively, vs Fc control group by 1-way ANOVA.

FIG. 25 shows evaluation percentage of p15e tetramer+CD8+ T cells among total cells in the tumors from mice treated IP (intraperitoneal) or IT (intratumoral injections) with variant CD80 IgV-Fc. *, *** p<0.05 or 0.001, respectively, vs Fc control group by 1-way ANOVA.

FIG. 26A-26B shows results from assessment of blocking of the PD-L1/PD-1 and CTLA-4/CD80 interaction by exemplary multivalent variant CD80 IgSF domain fusion proteins.

FIG. 27 shows IL-2 production in an assessment of Cytomegalovirus (CMV) antigen specific T cell response with exemplary multivalent variant CD80 IgSF domain fusion proteins.

FIG. 28A shows observed (circles) and predicted (mouse PK model; solid lines) serum concentration in control mice (non-tumor bearing) for dose groups over days.

FIG. 28B shows the goodness of fit for the mouse PK model. The top left scatter plot compares observations of serum concentration against predicted values at the population level. The top right scatter plot compares observations of serum concentration against predicted values at the individual level. In both plots, the dotted line represents unity. The bottom left and right plots show the distribution of weighted residuals for population predictions and time.

FIGS. 29A-29F show model predicted serum concentration values (median and confidence intervals (CI)) compared to observed serum concentration values in a mouse tumor model (murine colon adenocarcinoma MC38 cells expressing human PD-L1) where the animals have been treated. Data and prediction for groups of mice treated with CD80 IgV-Fc (H18Y/A26E/E35D/M47L/V68M/A71G/D90G) are shown for following dosages: a single dose of 100 μg (FIG. 29A; median and 80% CI), a single dose of 33 μg every 7 days (Q7D) for a total of 3 doses (FIG. 29B; median and 90% CI), a single dose of 100 μg (FIG. 29C; median and 90% CI), a single dose of 500 μg (FIG. 29D; median and 90% CI), single dose of 1500 μg (FIG. 29E; median and 90% CI), and a single dose of 167 μg every 3 days (Q3D) for a total of 3 doses (FIG. 29F; median and 90% CI). All treatments were administered intraperitoneal (IP).

FIG. 30A shows observed (circles) and predicted (monkey PK model; solid lines) serum concentration in cynomolgus monkeys for dose groups over days.

FIG. 30B shows the goodness of fit for the monkey PK model. The top left scatter plot compares observations of serum concentration against predicted values at the population level. The top right scatter plot compares observations of serum concentration against predicted values at the individual level. In both plots, the dotted line represents unity. The bottom left and right plots show the distribution of weighted residuals for population predictions and time.

FIGS. 31A-31B show observed (triangles and line fit) and predicted (mouse PD model; solid lines; PRED) tumor volume in hPD-L1-MC38 tumor bearing mice across different treatment groups over days. FIG. 31A shows study #1 treatment groups, where tumor-bearing mice received no treatment (CTRL), 33 μg of the exemplary tested CD80 IgV-Fc (H18Y/A26E/E35D/M47L/V68M/A71G/D90G) every 7 days for a total of 3 doses (Q7Dx3), or a single dose of 100 μg of CD80 IgV-Fc (H18Y/A26E/E35D/M47L/V68M/A71G/D90G). FIG. 31B shows study #2, where tumor-bearing mice received no treatment (CTRL), a single dose of 100 μg of CD80 IgV-Fc (H18Y/A26E/E35D/M47L/V68M/A71G/D90G), 167 μg of CD80 IgV-Fc (H18Y/A26E/E35D/M47L/V68M/A71G/D90G) every 3 days for a total of 3 doses (Q3Dx3), a single dose of 500 μg of CD80 IgV-Fc (H18Y/A26E/E35D/M47L/V68M/A71G/D90G), a or single dose of 1500 μg of the tested CD80 variant.

FIG. 32A shows predicted target (CD28) saturation in humans administered (intravenous injection (IV)) once weekly (Q1W) a dose of CD80 IgV-Fc (H18Y/A26E/E35D/M47L/V68M/A71G/D90G) at different concentrations.

FIG. 32B shows predicted human serum concentration levels of the drug under a regimen where the human was administered (IV) once weekly (Q1W) a dose of CD80 IgV-Fc (H18Y/A26E/E35D/M47L/V68M/A71G/D90G) at different concentrations.

FIG. 32C shows predicted human serum concentration levels of the drug under a regimen where the human was administered (IV) once every three weeks (Q3W) a dose of CD80 IgV-Fc (H18Y/A26E/E35D/M47L/V68M/A71G/D90G) at different concentrations.

FIG. 33 shows the changes in tumor volume for huPD-L1+MC38 tumor-bearing mice after treatment with a CD80 IgV-Fc variant, oxaliplatin, or both in combination.

FIG. 34 shows the changes in tumor volume for huPD-L1+MC38 tumor-bearing mice after treatment with a CD80 IgV-Fc variant, an anti-mouse checkpoint antibody against CTLA-4, or both in combination.

FIG. 35 shows the crystal structure of the binding interface between the CD80 IgV domain of a CD80 IgV-Fc variant and wild-type PD-L1.

FIG. 36A shows the changes in tumor volume for huPD-L1+MC38 tumor-bearing mice after treatment with a CD80 IgV-Fc variant, an anti-CD28 blocking antibody, or both in combination.

FIG. 36B shows the changes in tumor volume for huPD-L1+MC38 tumor-bearing mice after treatment with a CD80 IgV-Fc variant, an anti-PD-L1 blocking antibody, or both in combination.

FIG. 37 shows CD80 IgV-Fc secreted immunomodulatory protein (SIP) concentration levels over time in supernatant collected from SIP-transduced donor Pan T-cells.

FIG. 38 shows dose-dependent CD28 costimulation induced by exemplary CD80 IgV-Fc SIPs in a Jurkat/IL-2 reporter line.

FIG. 39 shows CD80 IgV-Fc SIP binding to PD-L1-expressing artificial antigen-presenting cells.

FIG. 40 depicts dose-dependent FcR-dependent CD28 agonism in a Jurkat/IL-2 reporter line induced by exemplary CD80 ECD-Fc variants.

### DETAILED DESCRIPTION

Provided herein are immunomodulatory proteins that are or contain variants or mutants of CD80 and specific binding fragments thereof that exhibit altered binding activity or affinity to at least one target ligand cognate binding partner (also called counter-structure ligand protein). In some embodiments, the variant CD80 polypeptides contain one or more amino acid modifications (e.g., amino acid substitutions, deletions, or additions) compared to an unmodified or wild-type CD80 polypeptide. In some embodiments, the variant CD80 polypeptides contain one or more amino acid modifications (e.g., substitutions) compared to an unmodified or wild-type CD80 polypeptide. In some embodiments, the one or more amino acid substitutions are in an IgSF domain (e.g., IgV) of an unmodified or wild-type CD80 polypeptide.

Also provided herein are immunomodulatory proteins that are fusion proteins that contain variants or mutants of the extracellular domain of CD80 and a multimerization domain. In some aspects, the provided variant CD80 fusion proteins contain a CD80 extracellular domain polypeptide with one or more amino acid modifications (e.g. substitutions) that confer altered binding activity or affinity to at least one target ligand cognate binding partner (also called counter-structure ligand protein). In some embodiments, the variant CD80 polypeptides contain one or more amino acid modifications (e.g., amino acid substitutions, deletions, or additions) compared to the extracellular domain of an unmodified or wild-type CD80 polypeptide. Methods of making and using these variants CD80 are also provided.

In some embodiments, the altered binding activity, such as binding affinity and/or binding selectivity, e.g., increased or decreased binding affinity or selectivity, is for at least one binding partner protein CD28, PD-L1, or CTLA-4. In some embodiments, the variant CD80 polypeptides exhibit altered, such as increased or decreased, binding activity or affinity to one or more of CD28, PD-L1, or CTLA-4 compared to the unmodified or wild-type CD80 not containing the one or more modifications.

In some embodiments, the variant CD80 polypeptides exhibit increased binding affinity to one or more of CD28, PD-L1, and CTLA-4 compared to the unmodified or wild-type CD80 not containing the one or more modifications. In some embodiments, the variant CD80 polypeptides exhibit increased binding affinity to CD28 compared to the unmodified or wild-type CD80 not containing the one or more modifications. In some embodiments, the variant CD80 polypeptides exhibit increased binding affinity to PD-L1 compared to the unmodified or wild-type CD80 not containing the one or more modifications. In some embodiments, the variant CD80 polypeptides exhibit increased binding affinity to CTLA-4 compared to the unmodified or wild-type CD80 not containing the one or more modifications.

In some embodiments, the variant CD80 polypeptides exhibit increased binding affinity to one or both of CD28 and PD-L1 compared to the unmodified or wild-type CD80 not containing the one or more modifications. In some embodiments, the variant CD80 polypeptides exhibit increased binding affinity to one or both of CD28 and CTLA-4 compared to the unmodified or wild-type CD80 not containing the one or more modifications. In some embodiments, the variant CD80 polypeptides exhibit increased binding affinity to one or both of PD-L1 and CTLA-4 compared to the unmodified or wild-type CD80 not containing the one or more modifications. In some embodiments, the variant CD80 polypeptides exhibit increased binding affinity to CD28, PD-L1 and CTLA-4 compared to the unmodified or wild-type CD80 not containing the one or more modifications.

In some embodiments, the variant CD80 polypeptides provided herein exhibit increased selectivity for binding to CD28, PD-L1 and/or CTLA-4 compared to the selectivity of the unmodified or wild-type CD80 not containing the one more modifications for binding to CD28, PD-L1 and/or CTLA-4. In some embodiments, the ratio is increased greater than or greater than about 1.2-fold, 1.5-fold, 2.0-fold, 3.0-fold, 4.0-fold, 5.0-fold, 6.0-fold, 7.0-fold, 8.0-fold, 9.0-fold, 10.0-fold, 15.0-fold, 20-fold, 30-fold, 40-fold, 50-fold, 100-fold or more.

In some embodiments, the variant CD80 polypeptides and immunomodulatory proteins modulate an immunological immune response, such as increase an immune response. In some embodiments, the provided variant CD80 polypeptides modulate T cell activation, expansion, differentiation, and survival via interactions with costimulatory signaling molecules. In general, antigen specific T-cell activation generally requires two distinct signals. The first signal is provided by the interaction of the T-cell receptor (TCR) with major histocompatibility complex (MHC) associated antigens present on antigen presenting cells (APCs). The second signal is costimulatory, e.g., a CD28 costimulatory signal, to TCR engagement and necessary to avoid T-cell apoptosis or anergy.

In some embodiments, under normal physiological conditions, the T cell-mediated immune response is initiated by antigen recognition by the T cell receptor (TCR) and is regulated by a balance of co-stimulatory and inhibitory signals (e.g., immune checkpoint proteins). The immune system relies on immune checkpoints to prevent autoimmunity (i.e., self-tolerance) and to protect tissues from excessive damage during an immune response, for example during an attack against a pathogenic infection. In some cases, however, these immunomodulatory proteins can be dysregulated in diseases and conditions, including tumors, as a mechanism for evading the immune system.

In some embodiments, among known T-cell costimulatory receptors is CD28, which is the T-cell costimulatory receptor for the ligands B7-1 (CD80) and B7-2 (CD86) both of which are present on APCs. These same ligands can also bind to the inhibitory T-cell receptor CTLA-4 (cytotoxic T-lymphocyte-associated protein 4) with greater affinity than for CD28; the binding to CTLA-4 acts to down-modulate the immune response. In some embodiments, CD80 is able to bind to programmed death ligand 1 (PD-L1). CD80 has similar affinity to PD-L1 as to CD28. PD-L1 is one of two ligands for the inhibitory immune receptor, programmed death 1 (PD-1). The interaction of PD-L1 with PD-1 negatively regulates immune activity by promoting T cell inactivation and down-modulating T cell activity. PD-1 expression on T cells may be induced after T cells have been activated as a strategy to prevent over activity of T cells. Many tumor cells express PD-L1 on their surface, potentially leading to PD-1/PD-L1 interactions and the inhibition of T cell responses against the tumor. The binding of CD80 to PD-L1 can block the interaction between PD-L1 and PD-1, and thereby prevent inhibition of T cell responses, e.g., at the site of a tumor, and effectively potentiate or enhance the immune response. In some embodiments, the provided CD80 polypeptides, e.g., soluble forms of the variant CD80 polypeptides provided herein, can antagonize B7/CTLA-4 binding, preventing CTLA-4 inhibitory signaling, reducing the TCR signaling threshold, thereby promoting T cell activation and immune response

In some embodiments, CD80 might be available to bind to CD28 receptors, and be involved in inducing T cell responses. In some embodiments, CD80 might be available to bind to PD-L1 to block the interaction between PD-L1 and PD-1 preventing inhibition of T cell responses or CTLA-4 to prevent CTLA-4 inhibitory signaling. Thus, in some cases, interactions of CD80 with PD-L1, CD28, and/or CTLA-4 can yield overlapping and complementary effects. In some embodiments, CD28 and PD-L1 may play complementary roles in modeling an immune response.

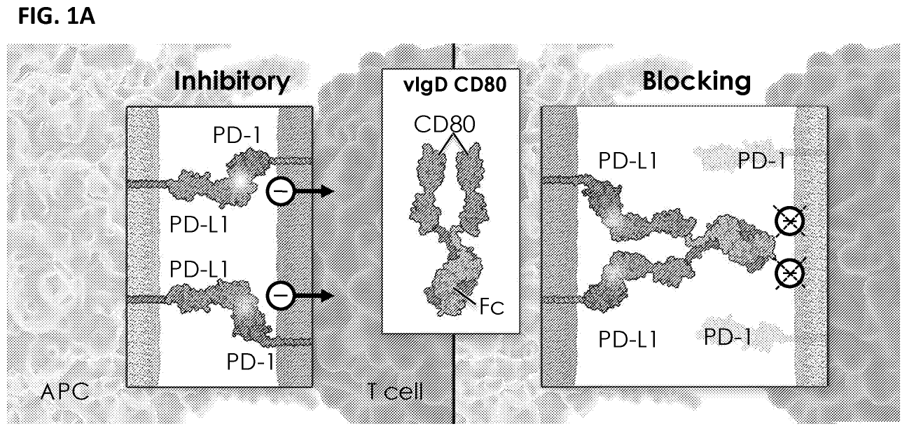

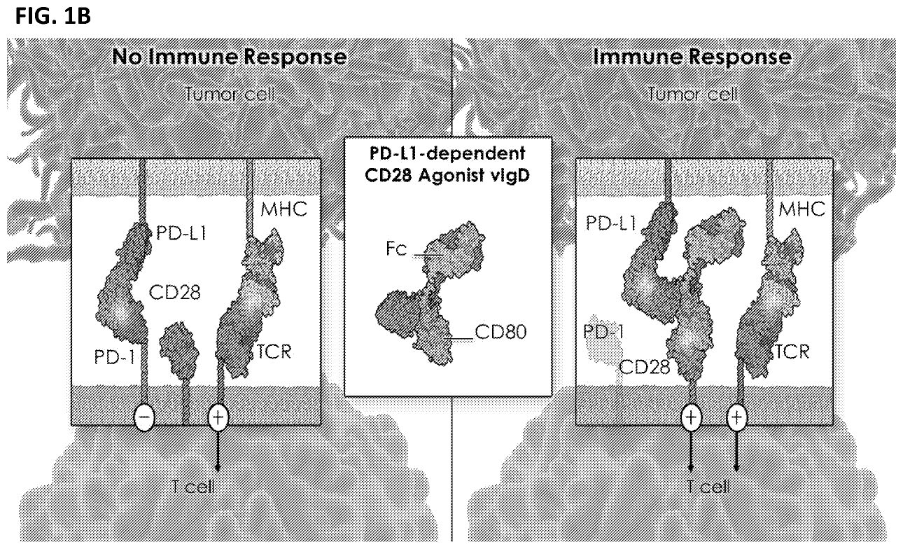

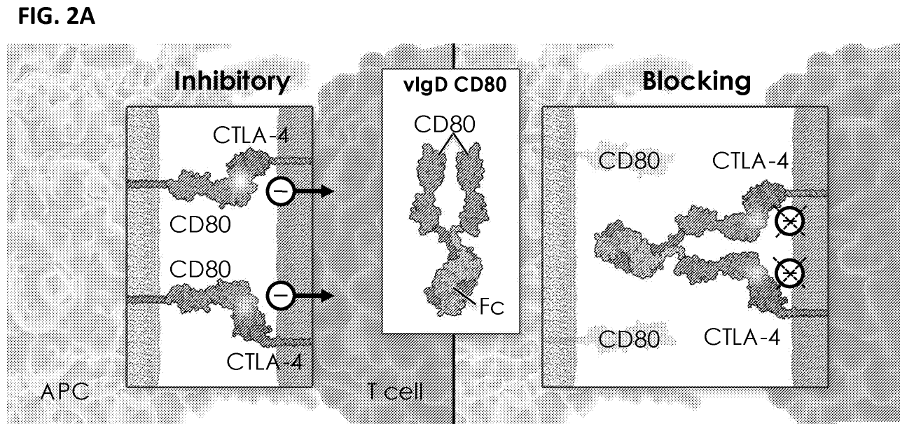

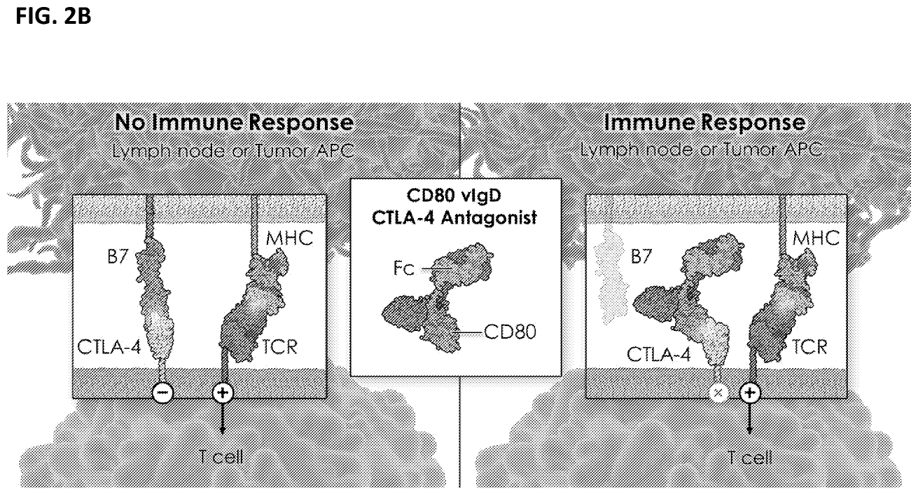

In some embodiments, the provided variant CD80 polypeptides or immunomodulatory proteins modulate (e.g., increase or decrease) immunological activity induced or associated with the inhibitory receptor CTLA-4, the PD-L1/PD-1 negative regulatory complex and/or the costimulatory receptor CD28. For example, in some embodiments, the provided CD80 polypeptides, e.g., soluble forms of the variant CD80 polypeptides provided herein, bind and co-stimulating a CD28 receptor on a localized T cell, thereby promoting an immune response. In some embodiments, the provided CD80 polypeptides, e.g., soluble forms of the variant CD80 polypeptides provided herein, are capable of binding the PD-L1 on a tumor cell or APC, thereby blocking the interaction of PD-L1 and the PD-1 inhibitory receptor, thereby preventing the negative regulatory signaling that would have otherwise resulted from the PD-L1/PD-1 interaction as depicted in in FIG. 1A. In some embodiments, the provided CD80 polypeptides, e.g., soluble forms of the variant CD80 polypeptides provided herein, bind the CTLA-4 inhibitory receptor, blocking its interaction with CD80, expressed on an APC, thereby preventing the negative regulatory signaling of the CD80-bound CTLA-4 receptor as depicted in in FIG. 2A. In some embodiments, the provided CD80 polypeptides, e.g., soluble forms of the variant CD80 polypeptides provided herein, can block the PD-L1/PD-1 interaction while, binding and co-stimulating a CD28 receptor on a localized T cell, thereby promoting an immune response (FIG. 1B). In some particular embodiments, the provided CD80 polypeptides, e.g., soluble forms of the variant CD80 polypeptides provided herein, also bind the CTLA-4 inhibitory receptor, blocking its interaction with CD80 and preventing the negative regulatory signaling of the CD80-bound CTLA-4 receptor.

Thus, in some embodiments, the provided polypeptides with independent binding affinities to both CD28 and/or PD-L1, and, in some cases, CTLA-4, thereby agonizing or antagonizing the complementary effects of costimulation by receptors. Methods of making and using these variants CD80 are also provided.

In some embodiments, the variant CD80 polypeptides specifically bind CD28 and/or CTLA-4, such as to human CD28 or human CTLA-4. In some embodiments, the variant CD80 polypeptides exhibit altered, such as increased, binding activity or affinity to one or both of CD28 or CTLA-4 compared to the unmodified or wild-type CD80 not containing the one or more modifications. In some embodiments, the variant CD80 polypeptides exhibit increased binding to CTLA-4, such as to human CTLA-4, compared to a wild-type human CD80 extracellular domain polypeptide. In some embodiments the variant CD80 polypeptides exhibit increased binding to CD28, such as to human CD28, compared to a wild-type human CD80 extracellular domain polypeptide.

In some embodiments, the variant CD80 IgSF domain fusion proteins are soluble. The ability to format the variant polypeptides in various configurations to, depending on the context, antagonize or agonize an immune response, offers flexibility in therapeutic applications based on the same increased binding and activity of a variant CD80 for binding partners. For example, delivery of enhanced CD80 protein in soluble formats with increased affinity for CD28, PD-L1 and/or CTLA-4 can antagonize signaling of an inhibitory receptor, such as block an inhibitory signal in the cell that may occur to decrease response to an activating stimulus, e.g., CD3 and/or CD28 costimulatory signal or a mitogenic signal. In some cases, the result of this can be to increase the immune response.

Additionally, certain formats, in some cases, also can mediate CD28 agonism. Among provided embodiments are embodiments that modulate, such as agonize, the costimulatory signal via CD28.

In some cases, CD28 agonism is mediated by certain variant CD80 polypeptides exhibiting increased binding to PD-L1 to thereby facilitate tethering or crosslinking of the variant CD80 molecule to a surface at the immune synapse for interaction with CD28, thereby facilitating T cell activation by providing a costimulatory signal. This activity, designated herein as PD-L1-dependent CD28 costimulation, is due, in some aspects, to the ability of a variant CD80 polypeptide to bind both PD-L1 and CD80 in a non-competitive manner and/or by provision of a dimeric format of a variant CD80 polypeptide (see e.g. FIG. 1B). In some cases, such PD-L1-dependent costimulation does not require an Fc with effector function and can be mediated by an Fc fusion protein containing an effector-less or inert Fc molecule. In some aspects, tethering or crosslinking also, additionally or alternatively, can be achieved via the Fc receptor when a variant CD80 polypeptide is provided as a fusion protein with a wild-type Fc region of an immunoglobulin that retains or exhibits effector function, designated herein as Fc receptor-dependent CD28 costimulation.

In some embodiments, it is found herein that certain formats of a variant full extracellular domain of a CD80 polypeptide can mediate CD28 agonism when formatted as a fusion protein with an immunoglobulin Fc that has effector activity. In such examples, binding of the variant CD80 fusion to an FcR via Fc binding may localize or tether the molecule to the immune synapse for engagement with CD28 on a T cell. In some aspects, it is contemplated that such activity is particularly effective in embodiments in which the CD80 polypeptide does not bind to programmed death ligand 1 (PD-L1). It has been reported that CD80 can bind to PD-L1. It is found that certain variants, and variants in certain formats such as formatted with the full extracellular domain of wild-type CD80, exhibit substantially lower PD-L1 binding or do not bind PD-L1. In some embodiments, a molecule that does not bind to PD-L1 exhibits background binding or only slightly above background binding to PD-L1 as detected in a binding assays, e.g. flow cytometry-based assay.

In some embodiments, the provided variant CD80 polypeptides exhibit increased binding to CD28. In some embodiments, increased binding to CD28 can result in an increase in CD28 costimulatory signaling, thereby promoting T cell activation and immune response. In some aspects, the increase in CD28 costimulatory signaling is dependent on an effector Fc that is able to bind to the FcR. In contrast, CD80 variants that bind PD-L1 can exhibit PD-L1-dependent CD28 agonism in formats that do not require an Fc with effector function, such as those in which the Fc fusion protein is an effector-less or inert Fc molecule.

In some aspects, crosslinking the Fc receptor, such as via its effector activity, can initiate antibody-dependent cell cytotoxicity (ADCC)-mediated effector functions, and thereby effect depletion of target cells expressing the cognate binding partner, such as CTLA-4-expressing cells (e.g. CTLA-4-expressing T regulatory cells) or PD-L1-expressing cells (e.g. PD-L1^{hi} tumors).

In some embodiments, the provided CD80 polypeptides, e.g., soluble forms of the variant CD80 polypeptides provided herein, can also antagonize B7/CTLA-4 binding, preventing CTLA-4 inhibitory signaling, reducing the TCR signaling threshold, thereby promoting T cell activation and immune response (FIG. 2B). In some embodiments, the provided CD80 polypeptides, e.g., soluble forms of the variant CD80 polypeptides provided herein, bind the CTLA-4 inhibitory receptor, blocking its interaction with CD80, expressed on an APC, thereby preventing the negative regulatory signaling of the CD80-bound CTLA-4 receptor as depicted in in FIGS. 2A and 2B.

In some embodiments, the provided variant CD80 polypeptides, such as variant CD80 fusion proteins, modulate, e.g. increase, immunological activity induced or associated with the inhibitory receptor CTLA-4, and/or the costimulatory receptor CD28.

Enhancement or suppression of the activity of these receptors has clinical significance for treatment of cancer. In some cases, however, therapies to intervene and alter the costimulatory effects of both receptors are constrained by the spatial orientation requirements as well as size limitations imposed by the confines of the immunological synapse. In some aspects, existing therapeutic drugs, including antibody drugs, may not be able to interact simultaneously with the multiple target proteins involved in modulating these interactions. In addition, in some cases, existing therapeutic drugs may only have the ability to antagonize, but not agonize, an immune response. Additionally, pharmacokinetic differences between drugs that independently target one or the other of these two receptors can create difficulties in properly maintaining a desired blood concentration of such drug combinations throughout the course of treatment. The provided variant CD80 polypeptides and immunomodulatory proteins, and other formats as described, address such problems.

All publications, including patents, patent applications scientific articles and databases, mentioned in this specification are herein incorporated by reference in their entirety for all purposes to the same extent as if each individual publication, including patent, patent application, scientific article or database, were specifically and individually indicated to be incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms used throughout this specification are defined as follows unless otherwise limited in specific instances. As used in the specification and the appended claims, the singular forms “a,” “an,” and “the” include plural referents unless the context clearly dictates otherwise. Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names are per IUPAC-IUB nomenclature. Unless indicated otherwise, all numerical ranges are inclusive of the values defining the range as well as all integer values in-between.

The term “affinity modified” as used in the context of an immunoglobulin superfamily domain, means a mammalian immunoglobulin superfamily (IgSF) domain having an altered amino acid sequence (relative to the corresponding wild-type parental or unmodified IgSF domain) such that it has an increased or decreased binding affinity or avidity to at least one of its cognate binding partners (alternatively “counter-structures”) compared to the parental wild-type or unmodified (i.e., non-affinity modified) IgSF control domain. Included in this context is an affinity modified CD80 IgSF domain. In some embodiments, the affinity-modified IgSF domain can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions, in a wildtype or unmodified IgSF domain. An increase or decrease in binding affinity or avidity can be determined using well known binding assays such as flow cytometry. Larsen et al., American Journal of Transplantation, Vol 5: 443-453 (2005). See also, Linsley et al., Immunity, Vol 1(9: 793-801 (1994). An increase in a protein's binding affinity or avidity to its cognate binding partner(s) is to a value at least 10% greater than that of the wild-type IgSF domain control and in some embodiments, at least 20%, 30%, 40%, 50%, 100%, 200%, 300%, 500%, 1000%, 5000%, or 10000% greater than that of the wild-type IgSF domain control value. A decrease in a protein's binding affinity or avidity to at least one of its cognate binding partner is to a value no greater than 90% of the control but no less than 10% of the wild-type IgSF domain control value, and in some embodiments no greater than 80%, 70% 60%, 50%, 40%, 30%, or 20% but no less than 10% of the wild-type IgSF domain control value. An affinity-modified protein is altered in primary amino acid sequence by substitution, addition, or deletion of amino acid residues. The term “affinity modified IgSF domain” is not to be construed as imposing any condition for any particular starting composition or method by which the affinity-modified IgSF domain was created. Thus, the affinity modified IgSF domains of the present invention are not limited to wild type IgSF domains that are then transformed to an affinity modified IgSF domain by any particular process of affinity modification. An affinity modified IgSF domain polypeptide can, for example, be generated starting from wild type mammalian IgSF domain sequence information, then modeled in silico for binding to its cognate binding partner, and finally recombinantly or chemically synthesized to yield the affinity modified IgSF domain composition of matter. In but one alternative example, an affinity modified IgSF domain can be created by site-directed mutagenesis of a wild-type IgSF domain. Thus, affinity modified IgSF domain denotes a product and not necessarily a product produced by any given process. A variety of techniques including recombinant methods, chemical synthesis, or combinations thereof, may be employed.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “antibody fragment” or “antigen-binding fragment” with reference to an antibody refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody.

The terms “binding affinity,” and “binding avidity” as used herein means the specific binding affinity and specific binding avidity, respectively, of a protein for its counter-structure under specific binding conditions. In biochemical kinetics, avidity refers to the accumulated strength of multiple affinities of individual non-covalent binding interactions, such as between CD80 and its counter-structures PD-L1, CD28, and/or CTLA-4. As such, avidity is distinct from affinity, which describes the strength of a single interaction. An increase or attenuation in binding affinity of a variant CD80 containing an affinity modified CD80 IgSF domain to its counter-structure is determined relative to the binding affinity of the unmodified CD80, such as an unmodified CD80 containing the native or wild-type IgSF domain, such as IgV domain. Methods for determining binding affinity or avidity are known in art. See, for example, Larsen et al., American Journal of Transplantation, Vol. 5: 443453 (2005). In some embodiments, a variant CD80, such as containing an affinity modified IgSF domain, specifically binds to CD28, PD-L1 and/or CTLA-4 measured by flow cytometry with a binding affinity that yields a Mean Fluorescence Intensity (MFI) value at least 10%, 20%, 30%, 40%, 50%, 60%, 70%, 80%, 90%, or 100% greater than an unmodified CD80 control in a binding assay such as described in Example 6.

The term “biological half-life” refers to the amount of time it takes for a substance, such as an immunomodulatory polypeptide containing a variant CD80 polypeptide of the present invention, to lose half of its pharmacologic or physiologic activity or concentration. Biological half-life can be affected by elimination, excretion, degradation (e.g., enzymatic) of the substance, or absorption and concentration in certain organs or tissues of the body. In some embodiments, biological half-life can be assessed by determining the time it takes for the blood plasma concentration of the substance to reach half its steady state level (“plasma half-life”). Conjugates that can be used to derivatize and increase the biological half-life of polypeptides of the invention are known in the art and include, but are not limited to, polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), polyglutamic acid (glutamylation).

The term “blocks binding,” and grammatical variations thereof, with reference to a PD-1 inhibitor, such as an anti-PD-1 antibody, refers to the ability of such inhibitor to inhibit or disrupt or reduce the interaction between PD-1 and a PD-1 ligand, such as PD-L1 or PD-L2. Such inhibition may occur through any mechanism, including direct interference with ligand binding, e.g., because of overlapping binding sites on PD-1, and/or conformational changes in PD-1 induced by the antibody that alter ligand affinity, etc.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. Examples of cancer include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

The term “chimeric antigen receptor” or “CAR” as used herein refers to an artificial (i.e., man-made) transmembrane protein expressed on a mammalian cell containing at least an ectodomain, a transmembrane, and an endodomain. Optionally, the CAR protein includes a “spacer” which covalently links the ectodomain to the transmembrane domain. A spacer is often a polypeptide linking the ectodomain to the transmembrane domain via peptide bonds. The CAR is typically expressed on a mammalian lymphocyte. In some embodiments, the CAR is expressed on a mammalian cell such as a T-cell or a tumor infiltrating lymphocyte (TIL). A CAR expressed on a T-cell is referred to herein as a “CAR T-cell” or “CAR-T.” In some embodiments the CAR-T is a T helper cell, a cytotoxic T-cell, a natural killer T-cell, a memory T-cell, a regulatory T-cell, or a gamma delta T-cell. When used clinically in, e.g., adoptive cell transfer, a CAR-T with antigen binding specificity to the patient's tumor is typically engineered to express on a native T-cell obtained from the patient. The engineered T-cell expressing the CAR is then infused back into the patient. The CAR-T is thus often an autologous CAR-T although allogeneic CAR-Ts are included within the scope of the invention. The ectodomain of a CAR contains an antigen binding region, such as an antibody or antigen binding fragment thereof (e.g., scFv), that specifically binds under physiological conditions with a target antigen, such as a tumor specific antigen Upon specific binding a biochemical chain of events (i.e., signal transduction) results in modulation of the immunological activity of the CAR-T. Thus, for example, upon specific binding by the antigen binding region of the CAR-T to its target antigen can lead to changes in the immunological activity of the T-cell activity as reflected by changes in cytotoxicity, proliferation or cytokine production. Signal transduction upon CAR-T activation is achieved in some embodiments by the CD3-zeta chain (“CD3-z”) which is involved in signal transduction in native mammalian T-cells. CAR-Ts can further contain multiple signaling domains such as CD28, 41BB or OX40, to further modulate immunomodulatory response of the T-cell. CD3-z contains a conserved motif known as an immunoreceptor tyrosine-based activation motif (ITAM) which is involved in T-cell receptor signal transduction.

The term “collectively” or “collective” when used in reference to cytokine production induced by the presence of two or more variant CD80 polypeptides in an in vitro assay, means the overall cytokine expression level irrespective of the cytokine production induced by individual variant CD80 polypeptides. In some embodiments, the cytokine being assayed is IFN-gamma in an in vitro primary T-cell assay such as described in Example 7.

The term “cognate binding partner” (used interchangeably with “counter-structure”) in reference to a polypeptide, such as in reference to an IgSF domain of a variant CD80, refers to at least one molecule (typically a native mammalian protein) to which the referenced polypeptide specifically binds under specific binding conditions. In some aspects, a variant CD80 containing an affinity modified IgSF domain specifically binds to the counter-structure of the corresponding native or wildtype CD80 but with increased or attenuated affinity. A species of ligand recognized and specifically binding to its cognate receptor under specific binding conditions is an example of a counter-structure or cognate binding partner of that receptor. A “cognate cell surface binding partner” is a cognate binding partner expressed on a mammalian cell surface. A “cell surface molecular species” is a cognate binding partner of ligands of the immunological synapse (IS), expressed on and by cells, such as mammalian cells, forming the immunological synapse.

As used herein, “conjugate,” “conjugation” or grammatical variations thereof refers the joining or linking together of two or more compounds resulting in the formation of another compound, by any joining or linking methods known in the art. It can also refer to a compound which is generated by the joining or linking together two or more compounds. For example, a variant CD80 polypeptide linked directly or indirectly to one or more chemical moieties or polypeptide is an exemplary conjugate. Such conjugates include fusion proteins, those produced by chemical conjugates and those produced by any other methods.

The term “competitive binding” as used herein means that a protein is capable of specifically binding to at least two cognate binding partners but that specific binding of one cognate binding partner inhibits, such as prevents or precludes, simultaneous binding of the second cognate binding partner. Thus, in some cases, it is not possible for a protein to bind the two cognate binding partners at the same time. Generally, competitive binders contain the same or overlapping binding site for specific binding but this is not a requirement. In some embodiments, competitive binding causes a measurable inhibition (partial or complete) of specific binding of a protein to one of its cognate binding partner due to specific binding of a second cognate binding partner. A variety of methods are known to quantify competitive binding such as ELISA (enzyme linked immunosorbent assay) assays.

As used herein, a composition refers to any mixture of two or more products, substances, or compounds, including cells. It may be a solution, a suspension, liquid, powder, a paste, aqueous, non-aqueous or any combination thereof.

The term “conservative amino acid substitution” as used herein means an amino acid substitution in which an amino acid residue is substituted by another amino acid residue having a side chain R group with similar chemical properties (e.g., charge or hydrophobicity). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine.

The term, “corresponding to” with reference to positions of a protein, such as recitation that nucleotides or amino acid positions “correspond to” nucleotides or amino acid positions in a disclosed sequence, such as set forth in the Sequence Listing, refers to nucleotides or amino acid positions identified upon alignment with the disclosed sequence based on structural sequence alignment or using a standard alignment algorithm, such as the GAP algorithm. For example, corresponding residues can be determined by alignment of a reference sequence with the sequence of wild-type CD80 set forth in SEQ ID NO: 2 (ECD domain) or set forth in SEQ ID NO: 76, 150, or 1245 (IgV domain) by structural alignment methods as described herein. By aligning the sequences, one skilled in the art can identify corresponding residues, for example, using conserved and identical amino acid residues as guides.

The terms “decrease” or “attenuate” “or suppress” as used herein means to decrease by a statistically significant amount. A decrease can be at least 10%, 20%, 30%, 40%, 50%, 60%, 70%, 80%, 90%, or 100%.

The terms “derivatives” or “derivatized” refer to modification of a protein by covalently linking it, directly or indirectly, to a composition so as to alter such characteristics as biological half-life, bioavailability, immunogenicity, solubility, toxicity, potency, or efficacy while retaining or enhancing its therapeutic benefit. Derivatives of immunomodulatory polypeptides of the invention are within the scope of the invention and can be made by, for example, glycosylation, PEGylation, lipidation, or Fc-fusion.

As used herein, detection includes methods that permit visualization (by eye or equipment) of a protein. A protein can be visualized using an antibody specific to the protein. Detection of a protein can also be facilitated by fusion of the protein with a tag including a label that is detectable or by contact with a second reagent specific to the protein, such as a secondary antibody, that includes a label that is detectable.

As used herein, domain (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs. In another example, a domain can be distinguished by its function, such as an ability to interact with a biomolecule, such as a cognate binding partner. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. Such domains are known, and can be identified by those of skill in the art. For exemplification herein, definitions are provided, but it is understood that it is well within the skill in the art to recognize particular domains by name. If needed appropriate software can be employed to identify domains.

The term “ectodomain” as used herein refers to the region of a membrane protein, such as a transmembrane protein, that lies outside the vesicular membrane. Ectodomains often contain binding domains that specifically bind to ligands or cell surface receptors, such as via a binding domain that specifically binds to the ligand or cell surface receptor. The ectodomain of a cellular transmembrane protein is alternately referred to as an extracellular domain.

The terms “effective amount” or “therapeutically effective amount” refer to a quantity and/or concentration of a therapeutic composition of the invention, including a protein composition or cell composition, that when administered ex vivo (by contact with a cell from a patient) or in vivo (by administration into a patient) either alone (i.e., as a monotherapy) or in combination with additional therapeutic agents, yields a statistically significant decrease in disease progression as, for example, by ameliorating or eliminating symptoms and/or the cause of the disease. An effective amount may be an amount that relieves, lessens, or alleviates at least one symptom or biological response or effect associated with a disease or disorder, prevents progression of the disease or disorder, or improves physical functioning of the patient. In some embodiments the patient is a mammal such as a non-human primate or human patient.

The term “endodomain” as used herein refers to the region found in some membrane proteins, such as transmembrane proteins, that extend into the interior space defined by the cell surface membrane. In mammalian cells, the endodomain is the cytoplasmic region of the membrane protein. In cells, the endodomain interacts with intracellular constituents and can be play a role in signal transduction and thus, in some cases, can be an intracellular signaling domain. The endodomain of a cellular transmembrane protein is alternately referred to as a cytoplasmic domain, which, in some cases, can be a cytoplasmic signaling domain.

The terms “enhanced” or “increased” as used herein in the context of increasing immunological activity of a mammalian lymphocyte means to increase one or more activities the lymphocyte. An increased activity can be one or more of increase cell survival, cell proliferation, cytokine production, or T-cell cytotoxicity, such as by a statistically significant amount. In some embodiments, reference to increased immunological activity means to increase interferon gamma (IFN-gamma) production, such as by a statistically significant amount. In some embodiments, the immunological activity can be assessed in a mixed lymphocyte reaction (MLR) assay. Methods of conducting MLR assays are known in the art. Wang et al., Cancer Immunol Res. 2014 September: 2(9):846-56. Other methods of assessing activities of lymphocytes are known in the art, including any assay as described herein. In some embodiments an enhancement can be an increase of at least 10%, 20%, 30%, 40%, 50%, 75%, 100%, 200%, 300%, 400%, or 500% greater than a non-zero control value.

The term “engineered cell” as used herein refers to a mammalian cell that has been genetically modified by human intervention such as by recombinant DNA methods or viral transduction. In some embodiments, the cell is an immune cell, such as a lymphocyte (e.g., T cell, B cell, NK cell) or an antigen presenting cell (e.g., dendritic cell). The cell can be a primary cell from a patient or can be a cell line. In some embodiments, an engineered cell of the invention contains a variant CD80 of the invention engineered to modulate immunological activity of a T-cell expressing CD28, PD-L1 and/or CTLA-4, or an APC expressing PD-L1, to which the variant CD80 polypeptide specifically binds.

The term “engineered T-cell” as used herein refers to a T-cell such as a T helper cell, cytotoxic T-cell (alternatively, cytotoxic T lymphocyte or CTL), natural killer T-cell, regulatory T-cell, memory T-cell, or gamma delta T-cell, that has been genetically modified by human intervention such as by recombinant DNA methods or viral transduction methods.

The term “engineered T-cell receptor” or “engineered TCR” refers to a T-cell receptor (TCR) engineered to specifically bind with a desired affinity to a major histocompatibility complex (MHC)/peptide target antigen that is selected, cloned, and/or subsequently introduced into a population of T-cells, often used for adoptive immunotherapy. In contrast to engineered TCRs, CARs are engineered to bind target antigens in a MHC independent manner.

The term “expressed on” as used herein is used in reference to a protein expressed on the surface of a cell, such as a mammalian cell. Thus, the protein is expressed as a membrane protein. In some embodiments, the expressed protein is a transmembrane protein. In some embodiments, the protein is conjugated to a small molecule moiety such as a drug or detectable label. Proteins expressed on the surface of a cell can include cell-surface proteins such as cell surface receptors that are expressed on mammalian cells.

The term “half-life extending moiety” refers to a moiety of a polypeptide fusion or chemical conjugate that extends the half-life of a protein circulating in mammalian blood serum compared to the half-life of the protein that is not so conjugated to the moiety. In some embodiments, half-life is extended by greater than or greater than about 1.2-fold, 1.5-fold, 2.0-fold, 3.0-fold, 4.0-fold, 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety. The half-life refers to the amount of time it takes for the protein to lose half of its concentration, amount, or activity. Half-life can be determined for example, by using an ELISA assay or an activity assay. Exemplary half-life extending moieties include an Fc domain, a multimerization domain, polyethylene glycol (PEG), hydroxyethyl starch (HES), XTEN (extended recombinant peptides; see, WO2013130683), human serum albumin (HSA), bovine serum albumin (BSA), lipids (acylation), and poly-Pro-Ala-Ser (PAS), and polyglutamic acid (glutamylation).

The term “immunological synapse” or “immune synapse” as used herein means the interface between a mammalian cell that expresses MHC I (major histocompatibility complex) or MHC II, such as an antigen-presenting cell or tumor cell, and a mammalian lymphocyte such as an effector T cell or Natural Killer (NK) cell.

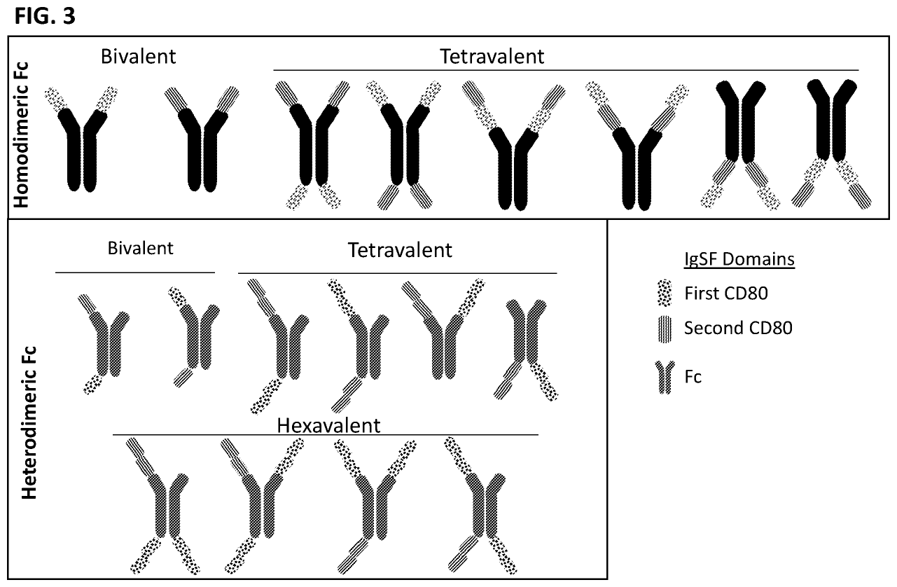

An Fc (fragment crystallizable) region or domain of an immunoglobulin molecule (also termed an Fc polypeptide) corresponds largely to the constant region of the immunoglobulin heavy chain, and is responsible for various functions, including the antibody's effector function(s). The Fc domain contains part or all of a hinge domain of an immunoglobulin molecule plus a CH2 and a CH3 domain. The Fc domain can form a dimer of two polypeptide chains joined by one or more disulfide bonds. Exemplary dimerized polypeptides are depicted in FIG. 3. In some embodiments, the Fc is a variant Fc that exhibits reduced (e.g., reduced greater than 30%, 40%, 50%, 60%, 70%, 80%, 90% or more) activity to facilitate an effector function. In some embodiments, reference to amino acid substitutions in an Fc region is by EU numbering system unless described with reference to a specific SEQ ID NO. EU numbering is known and is according to the most recently updated IMGT Scientific Chart (IMGT®, the international ImMunoGeneTics information System®, http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

An immunoglobulin Fc fusion (“Fc-fusion”), such as an immunomodulatory Fc fusion protein, is a molecule comprising one or more polypeptides (or one or more small molecules) operably linked to an Fc region of an immunoglobulin. An Fc-fusion may comprise, for example, the Fc region of an antibody (which facilitates pharmacokinetics) and a variant CD80 polypeptide. An immunoglobulin Fc region may be linked indirectly or directly to one or more variant CD80 polypeptides or small molecules (fusion partners). Various linkers are known in the art and can optionally be used to link an Fc to a fusion partner to generate an Fc-fusion. Fc-fusions of identical species can be dimerized to form Fc-fusion homodimers, or using non-identical species to form Fc-fusion heterodimers. In some embodiments, the Fc is a mammalian Fc such as a murine, rabbit or human Fc.

The term “host cell” refers to a cell that can be used to express a protein encoded by a recombinant expression vector. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include Chinese hamster ovary (CHO) cells or their derivatives such as Veggie CHO, DG44, Expi CHO, or CHOZN and related cell lines which grow in serum-free media or CHO strain DX-B11, which is deficient in DHFR. In some embodiments, a host cell can be a mammalian cell (e.g., a human cell, a monkey cell, a hamster cell, a rat cell, a mouse cell, or an insect cell).

The term “immunoglobulin” (abbreviated “Ig”) as used herein refers to a mammalian immunoglobulin protein including any of the five human classes of antibody: IgA (which includes subclasses IgA1 and IgA2), IgD, IgE, IgG (which includes subclasses IgG1, IgG2, IgG3, and IgG4), and IgM. The term is also inclusive of immunoglobulins that are less than full-length, whether wholly or partially synthetic (e.g., recombinant or chemical synthesis) or naturally produced, such as antigen binding fragment (Fab), variable fragment (Fv) containing V_{H} and V_{L}, the single chain variable fragment (scFv) containing V_{H} and V_{L} linked together in one chain, as well as other antibody V region fragments, such as Fab′, F(ab)₂, F(ab′)₂, dsFv diabody, Fc, and Fd polypeptide fragments. Bispecific antibodies, homobispecific and heterobispecific, are included within the meaning of the term.

The term “immunoglobulin superfamily” or “IgSF” as used herein means the group of cell surface and soluble proteins that are involved in the recognition, binding, or adhesion processes of cells. Molecules are categorized as members of this superfamily based on shared structural features with immunoglobulins (i.e., antibodies); they all possess a domain known as an immunoglobulin domain or fold. Members of the IgSF include cell surface antigen receptors, co-receptors and co-stimulatory molecules of the immune system, molecules involved in antigen presentation to lymphocytes, cell adhesion molecules, certain cytokine receptors and intracellular muscle proteins. They are commonly associated with roles in the immune system. Proteins in the immunological synapse are often members of the IgSF. IgSF can also be classified into “subfamilies” based on shared properties such as function. Such subfamilies typically consist of from 4 to 30 IgSF members.

The terms “IgSF domain” or “immunoglobulin domain” or “Ig domain” as used herein refers to a structural domain of IgSF proteins. Ig domains are named after the immunoglobulin molecules. They contain about 70-110 amino acids and are categorized according to their size and function. Ig-domains possess a characteristic Ig-fold, which has a sandwich-like structure formed by two sheets of antiparallel beta strands. Interactions between hydrophobic amino acids on the inner side of the sandwich and highly conserved disulfide bonds formed between cysteine residues in the B and F strands stabilize the Ig-fold. One end of the Ig domain has a section called the complementarity determining region that is important for the specificity of antibodies for their ligands. The Ig like domains can be classified (into classes) as: IgV, IgC1, IgC2, or IgI. Most Ig domains are either variable (IgV) or constant (IgC). IgV domains with 9 beta strands are generally longer than IgC domains with 7 beta strands. Ig domains of some members of the IgSF resemble IgV domains in the amino acid sequence, yet are similar in size to IgC domains. These are called IgC2 domains, while standard IgC domains are called IgC1 domains. T-cell receptor (TCR) chains contain two Ig domains in the extracellular portion; one IgV domain at the N-terminus and one IgC1 domain adjacent to the cell membrane. CD80 contains two Ig domains: IgV and IgC.

The term “IgSF species” as used herein means an ensemble of IgSF member proteins with identical or substantially identical primary amino acid sequence. Each mammalian immunoglobulin superfamily (IgSF) member defines a unique identity of all IgSF species that belong to that IgSF member. Thus, each IgSF family member is unique from other IgSF family members and, accordingly, each species of a particular IgSF family member is unique from the species of another IgSF family member. Nevertheless, variation between molecules that are of the same IgSF species may occur owing to differences in post-translational modification such as glycosylation, phosphorylation, ubiquitination, nitrosylation, methylation, acetylation, and lipidation. Additionally, minor sequence differences within a single IgSF species owing to gene polymorphisms constitute another form of variation within a single IgSF species as do wild type truncated forms of IgSF species owing to, for example, proteolytic cleavage. A “cell surface IgSF species” is an IgSF species expressed on the surface of a cell, generally a mammalian cell.

The term “immunological activity” as used herein in the context of mammalian lymphocytes such as T-cells refers to one or more cell survival, cell proliferation, cytokine production (e.g., interferon-gamma), or T-cell cytotoxicity activities. In some cases, an immunological activity can means their expression of cytokines, such as chemokines or interleukins. Assays for determining enhancement or suppression of immunological activity include the MLR (mixed lymphocyte reaction) assays measuring interferon-gamma cytokine levels in culture supernatants (Wang et al., Cancer Immunol Res. 2014 September: 2(9):846-56), SEB (staphylococcal enterotoxin B) T cell stimulation assay (Wang et al., Cancer Immunol Res. 2014 September: 2(9):846-56), and anti-CD3 T cell stimulation assays (Li and Kurlander, J Transl Med. 2010: 8: 104). Since T cell activation is associated with secretion of IFN-gamma cytokine, detecting IFN-gamma levels in culture supernatants from these in vitro human T cell assays can be assayed using commercial ELISA kits (Wu et al, Immunol Lett 2008 Apr. 15; 117(1): 57-62). Induction of an immune response results in an increase in immunological activity relative to quiescent lymphocytes. An immunomodulatory protein, such as a variant CD80 polypeptide containing an affinity modified IgSF domain, as provided herein can in some embodiments increase or, in alternative embodiments, decrease IFN-gamma (interferon-gamma) expression in a primary T-cell assay relative to a wild-type IgSF member or IgSF domain control. Those of skill will recognize that the format of the primary T-cell assay used to determine an increase in IFN-gamma expression will differ from that employed to assay for a decrease in IFN-gamma expression. In assaying for the ability of an immunomodulatory protein or affinity modified IgSF domain of the invention to decrease IFN-gamma expression in a primary T-cell assay, a Mixed Lymphocyte Reaction (MLR) assay can be used as described in Example 6. Conveniently, a soluble form of an affinity modified IgSF domain of the invention can be employed to determine its ability to antagonize and thereby decrease the IFN-gamma expression in a MLR as likewise described in Example 6. Alternatively, in assaying for the ability of an immunomodulatory protein or affinity modified IgSF domain of the invention to increase IFN-gamma expression in a primary T-cell assay, a co-immobilization assay can be used. In a co-immobilization assay, a T-cell receptor signal, provided in some embodiments by anti-CD3 antibody, is used in conjunction with a co-immobilized affinity modified IgSF domain, such as a variant CD80, to determine the ability to increase IFN-gamma expression relative to a wild-type IgSF domain control. Methods to assay the immunological activity of engineered cells, including to evaluate the activity of a variant CD80 transmembrane immunomodulatory protein, are known in the art and include, but are not limited to, the ability to expand T cells following antigen stimulation, sustain T cell expansion in the absence of re-stimulation, and anti-cancer activities in appropriate animal models. Assays also include assays to assess cytotoxicity, including a standard ⁵¹Cr-release assay (see e.g., Milone et al., (2009) Molecular Therapy 17: 1453-1464) or flow based cytotoxicity assays, or an impedance based cytotoxicity assay (Peper et al. (2014) Journal of Immunological Methods, 405:192-198).

An “immunomodulatory polypeptide” or “immunomodulatory protein” is a polypeptide or protein molecule that modulates immunological activity. By “modulation” or “modulating” an immune response is meant that immunological activity is either increased or decreased. An immunomodulatory protein can be a single polypeptide chain or a multimer (dimers or higher order multimers) of at least two polypeptide chains covalently bonded to each other by, for example, interchain disulfide bonds. Thus, monomeric, dimeric, and higher order multimeric polypeptides are within the scope of the defined term. Multimeric polypeptides can be homomultimeric (of identical polypeptide chains) or heteromultimeric (of non-identical polypeptide chains). An immunomodulatory protein can comprise a variant CD80 polypeptide.

The term “increase” as used herein means to increase by a statistically significant amount. An increase can be at least 5%, 10%, 20%, 30%, 40%, 50%, 75%, 100%, or greater than a non-zero control value.

An “isoform” of CD80 is one of a plurality of naturally occurring CD80 polypeptides that differ in amino acid sequence. Isoforms can be the product of splice variants of an RNA transcript expressed by a single gene, or the expression product of highly similar but different genes yielding a functionally similar protein such as may occur from gene duplication. As used herein, the term “isoform” of CD80 also refers to the product of different alleles of a CD80 gene.

As used herein, a “kit” refers to a combination of components, such as a combination of the compositions herein and another item for a purpose including, but not limited to, reconstitution, activation, and instruments/devices for delivery, administration, diagnosis, and assessment of a biological activity or property. Kits optionally include instructions for use.

The term “label” refers to a compound or composition which can be attached or linked, directly or indirectly to provide a detectable signal or that can interact with a second label to modify a detectable signal. The label can be conjugated directly or indirectly to a polypeptide so as to generate a labeled polypeptide. The label can be detectable by itself (e.g., radioisotope labels or fluorescent labels) or, in the case of an enzymatic label, can catalyze chemical alteration of a substrate compound composition which is detectable. Non-limiting examples of labels included fluorogenic moieties, green fluorescent protein, or luciferase.

The term “lymphocyte” as used herein means any of three subtypes of white blood cell in a mammalian immune system. They include natural killer cells (NK cells) (which function in cell-mediated, cytotoxic innate immunity), T cells (for cell-mediated, cytotoxic adaptive immunity), and B cells (for humoral, antibody-driven adaptive immunity). T cells include: T helper cells, cytotoxic T-cells, natural killer T-cells, memory T-cells, regulatory T-cells, or gamma delta T-cells. Innate lymphoid cells (ILC) are also included within the definition of lymphocyte.

The term “subject,” in some cases used interchangeably with patient or individual, is a mammal, such as a human or other animal, and typically is human. The terms “mammal” includes reference to at least one of a: human, chimpanzee, rhesus monkey, cynomolgus monkey, dog, cat, mouse, or rat.

The terms “mammal,” or “patient” specifically includes reference to at least one of a: human, chimpanzee, rhesus monkey, cynomolgus monkey, dog, cat, mouse, or rat.

The term “membrane protein” as used herein means a protein that, under physiological conditions, is attached directly or indirectly to a lipid bilayer. A lipid bilayer that forms a membrane can be a biological membrane such as a eukaryotic (e.g., mammalian) cell membrane or an artificial (i.e., man-made) membrane such as that found on a liposome. Attachment of a membrane protein to the lipid bilayer can be by way of covalent attachment, or by way of non-covalent interactions such as hydrophobic or electrostatic interactions. A membrane protein can be an integral membrane protein or a peripheral membrane protein. Membrane proteins that are peripheral membrane proteins are non-covalently attached to the lipid bilayer or non-covalently attached to an integral membrane protein. A peripheral membrane protein forms a temporary attachment to the lipid bilayer such that under the range of conditions that are physiological in a mammal, peripheral membrane protein can associate and/or disassociate from the lipid bilayer. In contrast to peripheral membrane proteins, integral membrane proteins form a substantially permanent attachment to the membrane's lipid bilayer such that under the range of conditions that are physiological in a mammal, integral membrane proteins do not disassociate from their attachment to the lipid bilayer. A membrane protein can form an attachment to the membrane by way of one layer of the lipid bilayer (monotopic), or attached by way of both layers of the membrane (polytopic). An integral membrane protein that interacts with only one lipid bilayer is an “integral monotopic protein”. An integral membrane protein that interacts with both lipid bilayers is an “integral polytopic protein” alternatively referred to herein as a “transmembrane protein”.

The terms “modulating” or “modulate” as used herein in the context of an immune response, such as a mammalian immune response, refer to any alteration, such as an increase or a decrease, of existing or potential immune responses that occurs as a result of administration of an immunomodulatory polypeptide comprising a variant CD80 of the present invention. Thus, it refers to an alteration, such as an increase or decrease, of an immune response as compared to the immune response that occurs or is present in the absence of the administration of the immunomodulatory protein comprising the variant CD80. Such modulation includes any induction, activation, suppression or alteration in degree or extent of immunological activity of an immune cell. Immune cells include B cells, T cells, NK (natural killer) cells, NK T cells, professional antigen-presenting cells (APCs), and non-professional antigen-presenting cells, and inflammatory cells (neutrophils, macrophages, monocytes, eosinophils, and basophils). Modulation includes any change imparted on an existing immune response, a developing immune response, a potential immune response, or the capacity to induce, regulate, influence, or respond to an immune response. Modulation includes any alteration in the expression and/or function of genes, proteins and/or other molecules in immune cells as part of an immune response. Modulation of an immune response or modulation of immunological activity includes, for example, the following: elimination, deletion, or sequestration of immune cells; induction or generation of immune cells that can modulate the functional capacity of other cells such as autoreactive lymphocytes, antigen presenting cells, or inflammatory cells; induction of an unresponsive state in immune cells (i.e., anergy); enhancing or suppressing the activity or function of immune cells, including but not limited to altering the pattern of proteins expressed by these cells. Examples include altered production and/or secretion of certain classes of molecules such as cytokines, chemokines, growth factors, transcription factors, kinases, costimulatory molecules, or other cell surface receptors or any combination of these modulatory events. Modulation can be assessed, for example, by an alteration in IFN-gamma (interferon gamma) expression relative to the wild-type or unmodified CD80 control in a primary T cell assay (see, Zhao and Ji, Exp Cell Res. 2016 Jan. 1; 340(1): 132-138). Modulation can be assessed, for example, by an alteration of an immunological activity of engineered cells, such as an alteration in in cytotoxic activity of engineered cells or an alteration in cytokine secretion of engineered cells relative to cells engineered with a wild-type CD80 transmembrane protein.

The term, a “multimerization domain” refers to a sequence of amino acids that promotes stable interaction of a polypeptide molecule with one or more additional polypeptide molecules, each containing a complementary multimerization domain (e.g., a first multimerization domain and a second multimerization domain), which can be the same or a different multimerization domain. The interactions between complementary multimerization domains, e.g., interaction between a first multimerization domain and a second multimerization domain, form a stable protein-protein interaction to produce a multimer of the polypeptide molecule with the additional polypeptide molecule. In some cases, the multimerization domain is the same and interacts with itself to form a stable protein-protein interaction between two polypeptide chains. Generally, a polypeptide is joined directly or indirectly to the multimerization domain. Exemplary multimerization domains include the immunoglobulin sequences or portions thereof, leucine zippers, hydrophobic regions, hydrophilic regions, and compatible protein-protein interaction domains. The multimerization domain, for example, can be an immunoglobulin constant region or domain, such as, for example, the Fc domain or portions thereof from IgG, including IgG1, IgG2, IgG3 or IgG4 subtypes, IgA, IgE, IgD and IgM and modified forms thereof.

The terms “nucleic acid” and “polynucleotide” are used interchangeably to refer to a polymer of nucleic acid residues (e.g., deoxyribonucleotides or ribonucleotides) in either single- or double-stranded form. Unless specifically limited, the terms encompass nucleic acids containing known analogues of natural nucleotides and that have similar binding properties to it and are metabolized in a manner similar to naturally-occurring nucleotides. Unless otherwise indicated, a particular nucleic acid sequence also implicitly encompasses conservatively modified variants thereof (e.g., degenerate codon substitutions) and complementary nucleotide sequences as well as the sequence explicitly indicated (a “reference sequence”). Specifically, degenerate codon substitutions may be achieved by generating sequences in which the third position of one or more selected (or all) codons is substituted with mixed-base and/or deoxyinosine residues. The term nucleic acid or polynucleotide encompasses cDNA or mRNA encoded by a gene.

The term “molecular species” as used herein means an ensemble of proteins with identical or substantially identical primary amino acid sequence. Each mammalian immunoglobulin superfamily (IgSF) member defines a collection of identical or substantially identical molecular species. Thus, for example, human CD80 is an IgSF member and each human CD80 molecule is a molecular species of CD80. Variation between molecules that are of the same molecular species may occur owing to differences in post-translational modification such as glycosylation, phosphorylation, ubiquitination, nitrosylation, methylation, acetylation, and lipidation. Additionally, minor sequence differences within a single molecular species owing to gene polymorphisms constitute another form of variation within a single molecular species as do wild type truncated forms of a single molecular species owing to, for example, proteolytic cleavage. A “cell surface molecular species” is a molecular species expressed on the surface of a mammalian cell. Two or more different species of protein, each of which is present exclusively on one or exclusively the other (but not both) of the two mammalian cells forming the IS, are said to be in “cis” or “cis configuration” with each other. Two different species of protein, the first of which is exclusively present on one of the two mammalian cells forming the IS and the second of which is present exclusively on the second of the two mammalian cells forming the IS, are said to be in “trans” or “trans configuration.” Two different species of protein each of which is present on both of the two mammalian cells forming the IS are in both cis and trans configurations on these cells.

The term “non-competitive binding” as used herein means the ability of a protein to specifically bind simultaneously to at least two cognate binding partners. Thus, the protein is able to bind to at least two different cognate binding partners at the same time, although the binding interaction need not be for the same duration such that, in some cases, the protein is specifically bound to only one of the cognate binding partners. In some embodiments, the binding occurs under specific binding conditions. In some embodiments, the simultaneous binding is such that binding of one cognate binding partner does not substantially inhibit simultaneous binding to a second cognate binding partner. In some embodiments, non-competitive binding means that binding a second cognate binding partner to its binding site on the protein does not displace the binding of a first cognate binding partner to its binding site on the protein. Methods of assessing non-competitive binding are well known in the art such as the method described in Perez de La Lastra et al., Immunology, 1999 April: 96(4): 663-670. In some cases, in non-competitive interactions, the first cognate binding partner specifically binds at an interaction site that does not overlap with the interaction site of the second cognate binding partner such that binding of the second cognate binding partner does not directly interfere with the binding of the first cognate binding partner. Thus, any effect on binding of the cognate binding partner by the binding of the second cognate binding partner is through a mechanism other than direct interference with the binding of the first cognate binding partner. For example, in the context of enzyme-substrate interactions, a non-competitive inhibitor binds to a site other than the active site of the enzyme. Non-competitive binding encompasses uncompetitive binding interactions in which a second cognate binding partner specifically binds at an interaction site that does not overlap with the binding of the first cognate binding partner but binds to the second interaction site only when the first interaction site is occupied by the first cognate binding partner.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical composition” refers to a composition suitable for pharmaceutical use in a mammalian subject, often a human. A pharmaceutical composition typically comprises an effective amount of an active agent (e.g., an immunomodulatory polypeptide comprising a variant CD80 or engineered cells expressing a variant CD80 transmembrane immunomodulatory protein) and a carrier, excipient, or diluent. The carrier, excipient, or diluent is typically a pharmaceutically acceptable carrier, excipient or diluent, respectively.

The terms “polypeptide” and “protein” are used interchangeably herein and refer to a molecular chain of two or more amino acids linked through peptide bonds. The terms do not refer to a specific length of the product. Thus, “peptides,” and “oligopeptides,” are included within the definition of polypeptide. The terms include post-translational modifications of the polypeptide, for example, glycosylation, acetylation, phosphorylation and the like. The terms also include molecules in which one or more amino acid analogs or non-canonical or unnatural amino acids that can be synthesized, or expressed recombinantly using known protein engineering techniques. In addition, proteins can be derivatized.

The term “primary T-cell assay” as used herein refers to an in vitro assay to measure interferon-gamma (“IFN-gamma”) expression. A variety of such primary T-cell assays are known in the art such as that described in Example 6. In a preferred embodiment, the assay used is anti-CD3 coimmobilization assay. In this assay, primary T cells are stimulated by anti-CD3 immobilized with or without additional recombinant proteins. Culture supernatants are harvested at timepoints, usually 24-72 hours. In another embodiment, the assay used is a mixed lymphocyte reaction (MLR). In this assay, primary T cells are simulated with allogenic APC. Culture supernatants are harvested at timepoints, usually 24-72 hours. Human IFN-gamma levels are measured in culture supernatants by standard ELISA techniques. Commercial kits are available from vendors and the assay is performed according to manufacturer's recommendation.

The term “purified” as applied to nucleic acids, such as encoding immunomodulatory proteins of the invention, generally denotes a nucleic acid or polypeptide that is substantially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or protein of the invention is at least about 50% pure, usually at least about 75%, 80%, 85%, 90%, 95%, 96%, 99% or more pure (e.g., percent by weight or on a molar basis).

The term “recombinant” indicates that the material (e.g., a nucleic acid or a polypeptide) has been artificially (i.e., non-naturally) altered by human intervention. The alteration can be performed on the material within, or removed from, its natural environment or state. For example, a “recombinant nucleic acid” is one that is made by recombining nucleic acids, e.g., during cloning, affinity modification, DNA shuffling or other well-known molecular biological procedures. A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid or that is otherwise altered by genetic engineering, such as by introducing into the cell a nucleic acid molecule encoding a recombinant protein, such as a transmembrane immunomodulatory protein provided herein. Transcriptional control signals in eukaryotes comprise “promoter” and “enhancer” elements. Promoters and enhancers consist of short arrays of DNA sequences that interact specifically with cellular proteins involved in transcription. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. The terms “in operable combination,” “in operable order” and “operably linked” as used herein refer to the linkage of nucleic acid sequences in such a manner or orientation that a nucleic acid molecule capable of directing the transcription of a given gene and/or the synthesis of a desired protein molecule is produced.

The term “recombinant expression vector” as used herein refers to a DNA molecule containing a desired coding sequence and appropriate nucleic acid sequences necessary for the expression of the operably linked coding sequence in a particular host cell. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the recombinant expression vector, operably linked to the coding sequence for the recombinant protein, such as a recombinant fusion protein, so that the expressed fusion protein can be secreted by the recombinant host cell, for easier isolation of the fusion protein from the cell, if desired. The term includes the vector as a self-replicating nucleic acid structure as well as the vector incorporated into the genome of a host cell into which it has been introduced. Among the vectors are viral vectors, such as lentiviral vectors.

The term “selectivity” refers to the preference of a subject protein, or polypeptide, for specific binding of one substrate, such as one cognate binding partner, compared to specific binding for another substrate, such as a different cognate binding partner of the subject protein. Selectivity can be reflected as a ratio of the binding activity (e.g., binding affinity) of a subject protein and a first substrate, such as a first cognate binding partner, (e.g., K_{d1}) and the binding activity (e.g., binding affinity) of the same subject protein with a second cognate binding partner (e.g., K_{d2}).

The term “sequence identity” as used herein refers to the sequence identity between genes or proteins at the nucleotide or amino acid level, respectively. “Sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. Methods for the alignment of sequences for comparison are well known in the art, such methods include GAP, BESTFIT, BLAST, FASTA and TFASTA. The BLAST algorithm calculates percent sequence identity and performs a statistical analysis of the similarity between the two sequences. The software for performing BLAST analysis is publicly available through the National Center for Biotechnology Information (NCBI) website.

The term “soluble” as used herein in reference to proteins, means that the protein is not a membrane protein. In general, a soluble protein contains only the extracellular domain of an IgSF family member receptor, or a portion thereof containing an IgSF domain or domains or specific-binding fragments thereof, but does not contain the transmembrane domain. In some cases, solubility of a protein can be improved by linkage or attachment, directly or indirectly via a linker, to an Fc domain, which, in some cases, also can improve the stability and/or half-life of the protein. In some aspects, a soluble protein is an Fc fusion protein.

The term “species” as used herein with respect to polypeptides or nucleic acids means an ensemble of molecules with identical or substantially identical sequences. Variation between polypeptides that are of the same species may occur owing to differences in post-translational modification such as glycosylation, phosphorylation, ubiquitination, nitrosylation, methylation, acetylation, and lipidation. Slightly truncated sequences of polypeptides that differ (or encode a difference) from the full length species at the amino-terminus or carboxyl-terminus by no more than 1, 2, or 3 amino acid residues are considered to be of a single species. Such microheterogeneities are a common feature of manufactured proteins.

The term “specific binding fragment” as used herein in reference to a full-length wild-type mammalian CD80 polypeptide or an IgV or an IgC domain thereof, means a polypeptide having a subsequence of an IgV and/or IgC domain and that specifically binds in vitro and/or in vivo to a mammalian CD28, mammalian PD-L1 and/or mammalian CTLA-4, such as a human or murine CD28, PD-L1, and/or CTLA-4. In some embodiments, the specific binding fragment of the CD80 IgV or the CD80 IgC is at least 60%, 70%, 75%, 80%, 85%, 90%, 95%, 96%, 97%, 98%, or 99% the sequence length of the full-length wild-type sequence. The specific binding fragment can be altered in sequence to form the variant CD80.

The term “specifically binds” as used herein means the ability of a protein, under specific binding conditions, to bind to a target protein such that its affinity or avidity is at least 5 times as great, but optionally at least 10, 20, 30, 40, 50, 100, 250 or 500 times as great, or even at least 1000 times as great as the average affinity or avidity of the same protein to a collection of random peptides or polypeptides of sufficient statistical size. A specifically binding protein need not bind exclusively to a single target molecule but may specifically bind to a non-target molecule due to similarity in structural conformation between the target and non-target (e.g., paralogs or orthologs). Those of skill will recognize that specific binding to a molecule having the same function in a different species of animal (i.e., ortholog) or to a non-target molecule having a substantially similar epitope as the target molecule (e.g., paralog) is possible and does not detract from the specificity of binding which is determined relative to a statistically valid collection of unique non-targets (e.g., random polypeptides). Thus, a polypeptide of the invention may specifically bind to more than one distinct species of target molecule due to cross-reactivity. Solid-phase ELISA immunoassays or surface plasmon resonance (e.g., Biacore) measurements can be used to determine specific binding between two proteins. Generally, interactions between two binding proteins have dissociation constants (K_{d}) less than 1×10⁻⁵ M, and often as low as 1×10⁻¹² M. In certain embodiments of the present disclosure, interactions between two binding proteins have dissociation constants of 1×10⁻⁶ M, 1×10⁻⁷ M, 1×10⁻⁸ M, 1×10⁻⁹ M, 1×10⁻¹⁰ M or 1×10⁻¹¹ M.

The terms “surface expresses” or “surface expression” in reference to a mammalian cell expressing a polypeptide means that the polypeptide is expressed as a membrane protein. In some embodiments, the membrane protein is a transmembrane protein.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods.

The term “targeting moiety” as used herein refers to a composition that is covalently or non-covalently attached to, or physically encapsulates, a polypeptide comprising the variant CD80. The targeting moiety has specific binding affinity for a desired counter-structure such as a cell surface receptor (e.g., the B7 family member PD-L1), or a tumor antigen such as tumor specific antigen (TSA) or a tumor associated antigen (TAA) such as B7-H6. Typically, the desired counter-structure is localized on a specific tissue or cell-type. Targeting moieties include: antibodies, antigen binding fragment (Fab), variable fragment (Fv) containing V_{H} and V_{L}, the single chain variable fragment (scFv) containing V_{H} and V_{L} linked together in one chain, as well as other antibody V region fragments, such as Fab′, F(ab)₂, F(ab′)₂, dsFv diabody, nanobodies, soluble receptors, receptor ligands, affinity matured receptors or ligands, as well as small molecule (<500 Dalton) compositions (e.g., specific binding receptor compositions). Targeting moieties can also be attached covalently or non-covalently to the lipid membrane of liposomes that encapsulate a polypeptide of the present invention.

The term “transmembrane protein” as used herein means a membrane protein that substantially or completely spans a lipid bilayer such as those lipid bilayers found in a biological membrane such as a mammalian cell, or in an artificial construct such as a liposome. The transmembrane protein comprises a transmembrane domain (“transmembrane domain”) by which it is integrated into the lipid bilayer and by which the integration is thermodynamically stable under physiological conditions. Transmembrane domains are generally predictable from their amino acid sequence via any number of commercially available bioinformatics software applications on the basis of their elevated hydrophobicity relative to regions of the protein that interact with aqueous environments (e.g., cytosol, extracellular fluid). A transmembrane domain is often a hydrophobic alpha helix that spans the membrane. A transmembrane protein can pass through the both layers of the lipid bilayer once or multiple times. A transmembrane protein includes the provided transmembrane immunomodulatory proteins described herein. In addition to the transmembrane domain, a transmembrane immunomodulatory protein of the invention further comprises an ectodomain and, in some embodiments, an endodomain.

The terms “treating,” “treatment,” or “therapy” of a disease or disorder as used herein mean slowing, stopping or reversing the disease or disorders progression, as evidenced by decreasing, cessation or elimination of either clinical or diagnostic symptoms, by administration of a therapeutic composition (e.g., containing an immunomodulatory protein) of the invention either alone or in combination with another compound as described herein. As used herein in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS). “Preventing,” “prophylaxis,” or “prevention” of a disease or disorder as used in the context of this invention refers to the administration of an immunomodulatory polypeptide, either alone or in combination with another compound, to prevent the occurrence or onset of a disease or disorder or some or all of the symptoms of a disease or disorder or to lessen the likelihood of the onset of a disease or disorder.

The term “tumor specific antigen” or “TSA” as used herein refers to a counter-structure that is present primarily on tumor cells of a mammalian subject but generally not found on normal cells of the mammalian subject. A tumor specific antigen need not be exclusive to tumor cells but the percentage of cells of a particular mammal that have the tumor specific antigen is sufficiently high or the levels of the tumor specific antigen on the surface of the tumor are sufficiently high such that it can be targeted by anti-tumor therapeutics, such as immunomodulatory polypeptides of the invention, and provide prevention or treatment of the mammal from the effects of the tumor. In some embodiments, in a random statistical sample of cells from a mammal with a tumor, at least 50% of the cells displaying a TSA are cancerous. In other embodiments, at least 60%, 70%, 80%, 85%, 90%, 95%, or 99% of the cells displaying a TSA are cancerous.

The term “variant” (also “modified” or mutant”) as used in reference to a variant CD80 means a CD80, such as a mammalian (e.g., human or murine) CD80 created by human intervention. The variant CD80 is a polypeptide having an altered amino acid sequence, relative to an unmodified or wild-type CD80. The variant CD80 is a polypeptide which differs from a wild-type CD80 isoform sequence by one or more amino acid substitutions, deletions, additions, or combinations thereof. For purposes herein, the variant CD80 contains at least one affinity modified domain, whereby one or more of the amino acid differences occurs in an IgSF domain (e.g., IgV domain). A variant CD80 can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid differences, such as amino acid substitutions. A variant CD80 polypeptide generally exhibits at least 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type or unmodified CD80, such as to the sequence of SEQ ID NO:1, a mature sequence thereof or a portion thereof containing the extracellular domain or an IgSF domain thereof. In some embodiments, a variant CD80 polypeptide exhibits at least 50%, 60%, 70%, 80%, 85%, 86%, 87%, 88%, 89%, 90%, 91%, 92%, 93%, 94%, 95%, 96%, 97%, 98%, 99% or more sequence identity to a corresponding wild-type or unmodified CD80 comprising the sequence set forth in SEQ ID NO: 2, SEQ ID NO: 76, or SEQ ID NO: 150, or SEQ ID NO: 1245.

Non-naturally occurring amino acids as well as naturally occurring amino acids are included within the scope of permissible substitutions or additions. A variant CD80 is not limited to any particular method of making and includes, for example, de novo chemical synthesis, de novo recombinant DNA techniques, or combinations thereof. A variant CD80 of the invention specifically binds to at least one or more of: CD28, PD-L1 and/or CTLA-4 of a mammalian species. In some embodiments, the altered amino acid sequence results in an altered (i.e., increased or decreased) binding affinity or avidity to CD28, PD-L1 and/or CTLA-4 compared to the unmodified or wild-type CD80 protein. An increase or decrease in binding affinity or avidity can be determined using well known binding assays such as flow cytometry. Larsen et al., American Journal of Transplantation, Vol 5: 443-453 (2005). See also, Linsley et al., Immunity, Vol 1(9): 793-801 (1994). An increase in variant CD80 binding affinity or avidity to CD28, PD-L1 and/or CTLA-4 can be a value at least 5% greater than that of the unmodified or wild-type CD80 and in some embodiments, at least 10%, 15%, 20%, 30%, 40%, 50%, 100% greater than that of the unmodified or wild-type CD80 control value. A decrease in CD80 binding affinity or avidity to CD28, PD-L1 and/or CTLA-4 is to a value no greater than 95% of the of the unmodified or wild-type CD80 control values, and in some embodiments no greater than 80%, 70% 60%, 50%, 40%, 30%, 20%, 10%, 5%, or no detectable binding affinity or avidity of the unmodified or wild-type CD80 control values. A variant CD80 polypeptide is altered in primary amino acid sequence by substitution, addition, or deletion of amino acid residues. The term “variant” in the context of variant CD80 polypeptide is not to be construed as imposing any condition for any particular starting composition or method by which the variant CD80 is created. A variant CD80 can, for example, be generated starting from wild type mammalian CD80 sequence information, then modeled in silico for binding to CD28, PD-L1 and/or CTLA-4, and finally recombinantly or chemically synthesized to yield the variant CD80. In but one alternative example, the variant CD80 can be created by site-directed mutagenesis of an unmodified or wild-type CD80. Thus, variant CD80 denotes a composition and not necessarily a product produced by any given process. A variety of techniques including recombinant methods, chemical synthesis, or combinations thereof, may be employed.

The term “wild-type” or “natural” or “native” as used herein is used in connection with biological materials such as nucleic acid molecules, proteins (e.g., CD80), IgSF members, host cells, and the like, refers to those which are found in nature and not modified by human intervention.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11: 1726).

### I. VARIANT CD80 IGSF DOMAIN FUSION PROTEINS

Provided herein are fusion proteins containing variant CD80 polypeptides that exhibit altered (increased or decreased) binding activity or affinity for one or more CD80 binding partners. In some embodiments, the CD80 binding partner is CD28, PD-L1, or CTLA-4. In some embodiments, the variant CD80 polypeptides exhibit altered (e.g. increased) binding activity or affinity for one or more CD80 binding partners. In some embodiments, the variant CD80 polypeptides exhibit altered (e.g. increased) binding activity or affinity for two or more CD80 binding partners. In some embodiments, the two or more CD80 binding partner is two or more of CD28, PD-L1, or CTLA-4. In some embodiments, the variant CD80 polypeptides exhibit altered (e.g. increased) binding activity or affinity for three CD80 binding partners. In some embodiments, the CD80 binding partner is CD28, PD-L1, andCTLA-4. In some embodiments, the variant CD80 polypeptide contains one or more amino acid modifications, such as one or more substitutions (alternatively, “mutations” or “replacements”), deletions or additions in an immunoglobulin superfamily (IgSF) domain (IgD) relative to a wild-type or unmodified CD80 polypeptide or a portion of a wild-type or unmodified CD80 containing the IgD or a specific binding fragment thereof. Thus, a provided variant CD80 polypeptide is or comprises a variant IgD (hereinafter called “vIgD”) in which the one or more amino acid modifications (e.g., substitutions) is in an IgD. In some embodiments, the variant CD80 is soluble and lacks a transmembrane domain.

In some embodiments, the variant CD80 polypeptides contain an extracellular domain containing an IgD that includes an IgV domain and an IgC domain. In some embodiments, the IgD can include the entire extracellular domain (ECD). In some embodiments, the IgD comprises an IgV domain or an IgC (e.g., IgC2) domain or specific binding fragment of the IgV domain or the IgC (e.g., IgC2) domain, or combinations thereof. In some embodiments, the IgD can be an IgV only, the combination of the IgV and IgC, including the entire extracellular domain (ECD), or any combination of Ig domains of CD80. Table 1 provides exemplary residues that correspond to IgV or IgC regions of CD80. In some embodiments, the variant CD80 polypeptide contains an IgV domain, or an IgC domain, or specific binding fragments thereof in which the at least one amino acid modification (e.g., substitution) is in the IgV domain or IgC domain or the specific binding fragment thereof. In some embodiments, the variant CD80 polypeptide contains an IgV domain or specific binding fragments thereof in which the at least one of the amino acid modifications (e.g., substitutions) is in the IgV domain or a specific binding fragment thereof. In some embodiments, by virtue of the altered binding activity or affinity, the altered IgV domain or IgC domain is an affinity modified IgSF domain.

TABLE 1
CD80 Domains and Sequences

| | NCBI | | | | | | |
|---|---|---|---|---|---|---|---|
| | Protein | | | | | | |
| | Accession | | | | | | |
| | Number/ | | | Cognate Cell | Amino Acid Sequence (SEQ ID NO) | | |
| IgSF | UniProtKB | IgSF Region | | Surface | Precursor | | |
| Member | Protein | & Domain | Other | Binding | (mature | | |
| (Synonym) | Identifier | Class | Domains | Partners | residues) | Mature | ECD |
| CD80 | NP_005182.1 | 35-135, 35- | S: 1-34, | CD28, | 1 | 1536 | 2 |
| (B7-1) | P33681 | 138, 37-138, | E: 35-242, | CTLA4, PD- | (35-288) | | |
| | | or 35-141 | T: 243- | L1 | | | |
| | | IgV, | 263, C: | | | | |
| | | 145-230, 154- | 264-288 | | | | |
| | | 232, or 142- | | | | | |
| | | 232 IgC | | | | | |

In some embodiments, the variant is modified in one more IgSF domains relative to the sequence of an unmodified CD80 sequence. In some embodiments, the unmodified CD80 sequence is a wild-type CD80. In some embodiments, the unmodified or wild-type CD80 has the sequence of a native CD80 or an ortholog thereof. In some embodiments, the unmodified CD80 is or comprises the extracellular domain (ECD) of CD80 or a portion thereof containing one or more IgSF domain (see Table 1). For example, an unmodified CD80 polypeptide is or comprises an IgV domain set forth as amino acids 35-135 of SEQ ID NO:1, amino acids 35-138 of SEQ ID NO: 1 (see SEQ ID NO: 1245), or amino acids 35-141 of SEQ ID NO: 1. In some cases, an unmodified CD80 polypeptide is or comprises an IgC domain set forth as amino acids 145-230 of SEQ ID NO:1 or amino acids 142-232 of SEQ ID NO:1. In some embodiments, the extracellular domain of an unmodified or wild-type CD80 polypeptide comprises an IgV domain and an IgC domain or domains. However, the variant CD80 polypeptide need not comprise both the IgV domain and the IgC domain or domains. In some embodiments, the variant CD80 polypeptide comprises or consists essentially of the IgV domain or a specific binding fragment thereof. In some embodiments, the variant CD80 polypeptide comprises or consists essentially of the IgC domain or specific binding fragments thereof. In some embodiments, the variant CD80 is soluble and lacks a transmembrane domain. In some embodiments, the variant CD80 further comprises a transmembrane domain and, in some cases, also a cytoplasmic domain.

In some embodiments, the wild-type or unmodified CD80 polypeptide is a mammalian CD80 polypeptide, such as, but not limited to, a human, a mouse, a cynomolgus monkey, or a rat CD80 polypeptide. In some embodiments, the wild-type or unmodified CD80 sequence is human.

In some embodiments, the wild-type or unmodified CD80 polypeptide has (i) the sequence of amino acids set forth in SEQ ID NO: 1 or a mature form thereof lacking the signal sequence, (ii) a sequence of amino acids that exhibits at least about 85%, 86%, 87%, 88%, 89%, 90%, 91%, 92%, 93%, 94%, 95%, 96%, 97%, 98%, 99% sequence identity to SEQ ID NO: 1 or a mature form thereof, or (iii) is a portion of (i) or (ii) containing an IgV domain or IgC domain or specific binding fragments thereof.

In some embodiments, the wild-type or unmodified CD80 polypeptide is or comprises an extracellular domain of the CD80 or a portion thereof. For example, in some embodiments, the unmodified or wild-type CD80 polypeptide comprises the amino acid sequence set forth in SEQ ID NO: 2, or an ortholog thereof. For example, the unmodified or wild-type CD80 polypeptide can comprise (i) the sequence of amino acids set forth in SEQ ID NO:2, (ii) a sequence of amino acids that has at least about 85%, 86%, 87%, 88%, 89%, 90%, 91%, 92%, 93%, 94%, 95%, 96%, 97%, 98%, 99% sequence identity to SEQ ID NO: 2, or (iii) is a specific binding fragment of (i) or (ii) comprising an IgV domain or an IgC domain. In some embodiments, the wild-type or unmodified extracellular domain of CD80 is capable of binding one or more CD80 binding proteins, such as one or more of CTLA-4, PD-L1 or CD28.

In some embodiments, the wild-type or unmodified CD80 polypeptide contains an IgV domain or an IgC domain, or a specific binding fragment thereof. In some embodiments, the IgV domain of the wild-type or unmodified CD80 polypeptide comprises the amino acid sequence set forth in SEQ ID NO: 76, 150, or 1245, or an ortholog thereof. For example, the IgV domain of the unmodified or wild-type CD80 polypeptide can contain (i) the sequence of amino acids set forth in SEQ ID NO: 76, 150, or 1245, (ii) a sequence of amino acids that has at least about 85%, 86%, 87%, 88%, 89%, 90%, 91%, 92%, 93%, 94%, 95%, 96%, 97%, 98%, 99% sequence identity to SEQ ID NO: 76, 150, or 1245, or (iii) is a specific binding fragment of (i) or (ii). In some embodiments, the wild-type or unmodified IgV domain is capable of binding one or more CD80 binding proteins, such as one or more of CTLA-4, PD-L1 or CD28.

In some embodiments, the IgC domain of the wild-type or unmodified CD80 polypeptide comprises the amino acid sequence set forth as residues 145-230, 154-232, or 142-232 of SEQ ID NO: 1, or an ortholog thereof. For example, the IgC domain of the unmodified or wild-type CD80 polypeptide can contain (i) the sequence of amino acids set forth as residues 145-230, 154-232, or 142-232 of SEQ ID NO: 1, (ii) a sequence of amino acids that has at least about 85%, 86%, 87%, 88%, 89%, 90%, 91%, 92%, 93%, 94%, 95%, 96%, 97%, 98%, 99% sequence identity to residues 145-230, 154-232, or 142-232 of SEQ ID NO: 1, or (iii) is a specific binding fragment of (i) or (ii). In some embodiments, the wild-type or unmodified IgC domain is capable of binding one or more CD80 binding proteins.

In some embodiments, the wild-type or unmodified CD80 polypeptide contains a specific binding fragment of CD80, such as a specific binding fragment of the IgV domain or the IgC domain. In some embodiments, the specific binding fragment can bind CD28, PD-L1 and/or CTLA-4. The specific binding fragment can have an amino acid length of at least 50 amino acids, such as at least 60, 70, 80, 90, 100, or 110 amino acids. In some embodiments, the specific binding fragment of the IgV domain contains an amino acid sequence that is at least about 85%, 86%, 87%, 88%, 89%, 90%, 91%, 92%, 93%, 94%, 95%, 96%, 97%, 98%, 99% of the length of the IgV domain set forth as amino acids 35-135, 35-138, 37-138 or 35-141 of SEQ ID NO: 1. In some embodiments, the specific binding fragment of the IgC domain comprises an amino acid sequence that is at least about 85%, 86%, 87%, 88%, 89%, 90%, 91%, 92%, 93%, 94%, 95%, 96%, 97%, 98%, 99% of the length of the IgC domain set forth as amino acids 145-230, 154-232, 142-232 of SEQ ID NO: 1.

In some embodiments, the variant CD80 IgSF domain fusion protein contains a variant CD80 polypeptide that comprises the ECD domain or a portion thereof comprising one or more affinity modified IgSF domains. In some embodiments, the variant CD80 polypeptides can comprise an IgV domain or an IgC domain, or a specific binding fragment of the IgV domain or a specific binding fragment of the IgC domain in which at least one of the IgV or IgC domain contains the one or more amino acid modifications (e.g., substitutions). In some embodiments, the variant CD80 polypeptides can comprise an IgV domain and an IgC domain, or a specific binding fragment of the IgV domain and a specific binding fragment of the IgC domain. In some embodiments, the variant CD80 polypeptide comprises a full-length IgV domain. In some embodiments, the variant CD80 polypeptide comprises a full-length IgC domain. In some embodiments, the variant CD80 polypeptide comprises a specific binding fragment of the IgV domain. In some embodiments, the variant CD80 polypeptide comprises a specific binding fragment of the IgC domain. In some embodiments, the variant CD80 polypeptide comprises a full-length IgV domain and a full-length IgC domain. In some embodiments, the variant CD80 polypeptide comprises a full-length IgV domain and a specific binding fragment of an IgC domain. In some embodiments, the variant CD80 polypeptide comprises a specific binding fragment of an IgV domain and a full-length IgC domain. In some embodiments, the variant CD80 polypeptide comprises a specific binding fragment of an IgV domain and a specific binding fragment of an IgC domain.

In any of such embodiments, the one or more amino acid modifications (e.g., substitutions) of the variant CD80 polypeptides can be located in any one or more of the CD80 polypeptide domains. For example, in some embodiments, one or more amino acid modifications (e.g., substitutions) are located in the extracellular domain of the variant CD80 polypeptide. In some embodiments, one or more amino acid modifications (e.g., substitutions) are located in the IgV domain or specific binding fragment of the IgV domain. In some embodiments, one or more amino acid modifications (e.g., substitutions) are located in the IgC domain or specific binding fragment of the IgC domain.

Generally, each of the various attributes of polypeptides are separately disclosed (e.g., affinity of CD80 for binding partners, number of variations per polypeptide chain, number of linked polypeptide chains, the number and nature of amino acid alterations per variant CD80, etc.). However, as will be clear to the skilled artisan, any particular polypeptide can comprise a combination of these independent attributes. It is understood that reference to amino acids, including to a specific sequence set forth as a SEQ ID NO used to describe domain organization of an IgSF domain are for illustrative purposes and are not meant to limit the scope of the embodiments provided. It is understood that polypeptides and the description of domains thereof are theoretically derived based on homology analysis and alignments with similar molecules. Thus, the exact locus can vary, and is not necessarily the same for each protein. Hence, the specific IgSF domain, such as specific IgV domain or IgC domain, can be several amino acids (such as one, two, three or four) longer or shorter.

Further, various embodiments of the invention as discussed below are frequently provided within the meaning of a defined term as disclosed above. The embodiments described in a particular definition are therefore to be interpreted as being incorporated by reference when the defined term is utilized in discussing the various aspects and attributes described herein. Thus, the headings, the order of presentation of the various aspects and embodiments, and the separate disclosure of each independent attribute is not meant to be a limitation to the scope of the present disclosure.

A. Variant CD80 Polypeptides

Provided herein are variant CD80 IgSF domain fusion proteins that contain at least one affinity-modified IgSF domain or a specific binding fragment thereof relative to an IgSF domain contained in a wild-type or unmodified CD80 polypeptide such that the variant CD80 polypeptide exhibits altered (increased or decreased) binding activity or affinity for one or more cognate binding partners, CD28, PD-L1, or CTLA-4, compared to a wild-type or unmodified CD80 polypeptide. In some embodiments, a variant CD80 polypeptide has a binding affinity for CD28, PD-L1, or CTLA-4 that differs from that of a wild-type or unmodified CD80 polypeptide control sequence as determined by, for example, solid-phase ELISA immunoassays, flow cytometry or surface plasmon resonance (Biacore) assays. In some embodiments, the variant CD80 polypeptide has an increased binding affinity for CD28, PD-L1, and/or CTLA-4. In some embodiments, the variant CD80 polypeptide has an increased binding affinity for CD28 and/or CTLA-4. In some embodiments, the variant CD80 polypeptide has an decreased binding affinity for PD-L1. The CD28, PD-L1 and/or the CTLA-4 can be a mammalian protein, such as a human protein or a murine protein.

The altered, e.g. increased, binding activity or affinity for CD28, PD-L1 and/or the CTLA-4 is conferred by one or more amino acid modifications in an IgSF domain of a wild-type or unmodified IgSF domain. The wild-type or unmodified CD80 sequence does not necessarily have to be used as a starting composition to generate variant CD80 polypeptides described herein. Therefore, use of the term “substitution” does not imply that the provided embodiments are limited to a particular method of making variant CD80 polypeptides. Variants CD80 polypeptides can be made, for example, by de novo peptide synthesis and thus does not necessarily require a “substitution” in the sense of altering a codon to encode for the substitution. This principle also extends to the terms “addition” and “deletion” of an amino acid residue which likewise do not imply a particular method of making. The means by which the variant CD80 polypeptides are designed or created is not limited to any particular method. In some embodiments, however, a wild-type or unmodified CD80 encoding nucleic acid is mutagenized from wild-type or unmodified CD80 genetic material and screened for desired specific binding affinity and/or induction of IFN-gamma expression or other functional activity according to the methods disclosed in the Examples or other methods known to a skilled artisan. In some embodiments, a variant CD80 polypeptide is synthesized de novo utilizing protein or nucleic acid sequences available at any number of publicly available databases and then subsequently screened. The National Center for Biotechnology Information provides such information and its website is publicly accessible via the internet as is the UniProtKB database as discussed previously.

Unless stated otherwise, as indicated throughout the present disclosure, the amino acid modifications(s) are designated by amino acid position number corresponding to the numbering of positions of the unmodified ECD sequence set forth in SEQ ID NO:2 or, where applicable, the unmodified IgV sequence set forth in SEQ ID NO: 76, 150, or 1245 as follows:

(SEQ ID NO: 2)
VIHVTKEVKEVATLSCGHNVSVEELAQTRIYWQKEKKMVL
TMMSGDMNIWPEYKNRTIEDITNNLSIVILALRPSDEGTY
ECVVLKYEKDAFKREHLAEVTLSVKADFPTPSISDFEIPT
SNIRRIICSTSGGFPEPHLSWLENGEELNAINTTVSQDPE
TELYAVSSKLDFNMTTNHSFMCLIKYGHLRVNQTFNWNTT
KQEHFPDN
(SEQ ID NO: 76)
VIHVTKEVKEVATLSCGHNVSVEELAQTRIYWQKEKKMVL
TMMSGDMNIWPEYKNRTIFDITNNLSIVILALRPSDEGTY
ECVVLKYEKDAFKREHLAEVT
(SEQ ID NO: 150)
VIHVTKEVKEVATLSCGHNVSVEELAQTRIYWQKEKKMVL
TMMSGDMNIWPEYKNRTIFDITNNLSIVILALRPSDEGTY
ECVVLKYEKDAFKREHLAEVTLSVKAD
(SEQ ID NO: 1245)
VIHVTKEVKEVATLSCGHNVSVEELAQTR1YWQKEKKMVL
TMMSGDMNIWPEYKNRTIFDITNNLSIVILALRPSDEGTY
ECVVLKYEKDAFKREHLAEVTLSV

It is within the level of a skilled artisan to identify the corresponding position of a modification, e.g., amino acid substitution, in a CD80 polypeptide, including portion thereof containing an IgSF domain (e.g., IgV) thereof, such as by alignment of a reference sequence with SEQ ID NO:2 or SEQ ID NO:76 or SEQ ID NO:150 or SEQ ID NO: 1245. In the listing of modifications throughout this disclosure, the amino acid position is indicated in the middle, with the corresponding unmodified (e.g., wild-type) amino acid listed before the number and the identified variant amino acid substitution listed after the number. If the modification is a deletion of the position, a “del” is indicated, and if the modification is an insertion at the position, an “ins” is indicated. In some cases, an insertion is listed with the amino acid position indicated in the middle, with the corresponding unmodified (e.g., wild-type) amino acid listed before and after the number and the identified variant amino acid insertion listed after the unmodified (e.g., wild-type) amino acid.

In particular embodiments provided herein, the amino acid modifications (e.g. substitutions) are in the full extracellular domain of a wild-type CD80. In some embodiments, the variant CD80 polypeptide contains amino acid residues corresponding to amino acid residues 35-230 of the exemplary wild-type human CD80 extracellular domain set forth in SEQ ID NO:1. In some embodiments, the variant CD80 polypeptides contains one or more amino acid substitutions in an extracellular domain corresponding to amino acid residues 35-230 of the exemplary wild-type human CD80 extracellular domain set forth in SEQ ID NO:1. In some embodiments, the extracellular domain of wild-type CD80 is set forth in SEQ ID NO:2. In some embodiments, the variant CD80 polypeptide containing the one or more amino acid substitutions in the extracellular domain has a sequence of amino acids that has at least or at least about 85%, 86%, 87%, 88%, 89%, 90%, 91%, 92%, 93%, 94%, 95%, 96%, 97%, 98%, 99% or more sequence identity to the sequence set forth in SEQ ID NO:2.

In some embodiments, the variant CD80 polypeptide has one or more amino acid modifications (e.g., substitutions) in a wild-type or unmodified CD80 sequence. The one or more amino acid modifications (e.g., substitutions) can be in the ectodomain (extracellular domain) of the wild-type or unmodified CD80 sequence, such as the extracellular domain. In some embodiments, the one or more amino acid modifications (e.g., substitutions) are in the IgV domain or specific binding fragment thereof. In some embodiments, the one or more amino acid modifications (e.g., substitutions) are in the IgC domain or specific binding fragment thereof. In some embodiments of the variant CD80 polypeptide, some of the one or more amino acid modifications (e.g., substitutions) are in the IgV domain or a specific binding fragment thereof, and some of the one or more amino acid modifications (e.g., substitutions) are in the IgC domain or a specific binding fragment thereof.

In some embodiments, the variant CD80 polypeptide has up to 1, 2, 3, 4, 5, 6, 7, 8, 9, 10, 11, 12, 13, 14, 15, 16, 17, 18, 19, or 20 amino acid modifications (e.g., substitutions). The modifications (e.g., substitutions) can be in the IgV domain or the IgC domain. In some embodiments, the variant CD80 polypeptide has up to 1, 2, 3, 4, 5, 6, 7, 8, 9, 10, 11, 12, 13, 14, 15, 16, 17, 18, 19, or 20 amino acid modifications (e.g., substitutions) in the IgV domain or specific binding fragment thereof. In some embodiments, the variant CD80 polypeptide has up to 1, 2, 3, 4, 5, 6, 7, 8, 9, 10, 11, 12, 13, 14, 15, 16, 17, 18, 19, or 20 amino acid modifications (e.g., substitutions) in the IgC domain or specific binding fragment thereof. In some embodiments, the variant CD80 polypeptide has at least about 85%, 86%, 86%, 88%, 89%, 90%, 91%, 92%, 93%, 94%, 95%, 96%, 97%, 98%, or 99% sequence identity with the wild-type or unmodified CD80 polypeptide or specific binding fragment thereof, such as the amino acid sequence of SEQ ID NO: 2, 76, 150, or 1245.

In some embodiments, the variant CD80 polypeptide has one or more amino acid modifications (e.g., substitutions) in an unmodified CD80 or specific binding fragment there of corresponding to position(s) 4, 7, 9, 10, 11, 12, 13, 14, 15, 16, 17, 18, 20, 21, 22, 23, 24, 25, 26, 27, 28, 29, 30, 31, 33, 34, 35, 36, 37, 38, 40, 41, 42, 43, 44, 46, 47, 48, 49, 50, 51, 52, 53, 54, 55, 57, 58, 59, 60, 61, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 99, 102, 103, 104, 107, 108, 109, 110, 114, 115, 116, 117, 118, 120, 121, 122, 126, 127, 128, 129, 130, 133, 137, 140, 142, 143, 144, 148, 149, 152, 154, 160, 162, 164, 168, 169, 174, 175, 177, 178, 182, 183, 185, 178, 185, 188, 190, 192, 193, or 199 with reference to numbering of SEQ ID NO: 2. In some embodiments, such variant CD80 polypeptides exhibit altered binding affinity to one or more of CD28, PD-L1, or CTLA-4 compared to the wild-type or unmodified CD80 polypeptide. For example, in some embodiments, the variant CD80 polypeptide exhibits increased binding affinity to CD28, PD-L1, and/or CTLA-4 compared to a wild-type or unmodified CD80 polypeptide.

In some embodiments, the variant CD80 polypeptide has one or more amino acid substitution selected from V4M, E7D, K9E, E10R, V11S, A12G, A12T, A12V, T13A, T13N, T13R, L14A, S15F, S15P, S15T, S15V, C16G, C16L, C16R, C16S, G17W, H18A, H18C, H18F, H18I, H18L, H18R, H18T, H18V, H18Y, V20A, V20I, V20L, S21P, V22A, V22D, V22I, V22L, E23D, E23G, E24D, E24G, L25P, L25S, A26D, A26E, A26G, A26H, A26K, A26N, A26P, A26Q, A26R, A26S, A26T, Q27H, Q27L, Q27R, T28A, T28S, T28Y, R29C, R29D, R29H, R29V, I30F, I30T, I30V, Y31C, Y31F, Y31H, Y31L, Y31S, Q33E, Q33H, Q33K, Q33L, Q33R, K34E, E35D, E35G, K36E, K36G, K36R, K37E, K37Q, M38I, M38L, M38T, M38V, L40M, T41A, T41D, T41G, T41I, T41S, M42I, M42T, M42V, M43I, M43L, M43Q, M43R, M43T, M43V, S44P, D46E, D46N, D46V, M47F, M47I, M47L, M47T, M47V, M47Y, N48D, N48H, N48I, N48K, N48R, N48S, N48T, N48Y, I49V, W50G, P51A, E52D, E52G, Y53C, Y53F, Y53H, K54E, K54M, K54N, K54R, N55D, N55I, T57A, T57I, I58V, F59L, F59S, D60V, I61F, I61N, I61V, T62A, T62N, T62S, N63D, N63S, N64S, L65H, L65P, S66H, I67F, I67L, I67T, I67V, V68A, V68E, V68I, V68L, V68M, I69F, I69T, L70M, L70P, L70Q, L70R, A71D, A71G, L72P, L72V, R73H, R73S, P74L, P74S, D76G, D76H, E77A, E77G, E77K, G78A, T79A, T79I, T79L, T79M, T79P, Y80N, E81A, E81G, E81K, E81R, E81V, C82R, V83A, V83I, V84A, V84I, L85E, L85I, L85M, L85Q, L85R, K86E, K86M, Y87C, Y87D, Y87H, Y87N, Y87Q, E88D, E88G, E88V, K89E, K89N, K89R, D90G, D90K, D90L, D90N, D90P, A91E, A91G, A91S, A91T, A91V, F92L, F92N, F92P, F92S, F92V, F92Y, K93I, K93E, K93Q, K93R, K93T, K93V, R94F, R94G, R94L, R94Q, R94W, E95D, E95K, E95V, H96R, L97M, L97R, L97Q, E99D, E99G, L102S, S103L, S103P, V104A, V104L, D107N, F108L, P109H, P109S, T110A, S114T, D115G, F116L, F116S, E117V, E117G, I118A, I118T, I118V, T120S, S121P, N122S, I126L, I126V, I127T, C128R, C128Y, S129L, S129P, T130A, G133D, P137L, S140T, L142S, E143G, N144D, N144S, L148S, N149D, N149S, N152T, T154A, T154I, E160G, E162G, Y164H, S168G, K169E, K169I, K169S, M174T, M174V, T175A, N177S, H178R, C182S, L183H, K185E, H188D, H188Q, R190S, N192D, Q193L, or T199S.

In some embodiments, the one or more amino acid modification, e.g. substitution is L70P, I30F/L70P, Q27H/T41S/A71D, I30T/L70R, T13R/C16R/L70Q/A71D, T57I, M43I/C82R, V22L/M38V/M47T/A71D/L85M, I30V/T57I/L70P/A71D/A91T, V22I/L70M/A71D, N55D/L70P/E77G, T57A/I69T, N55D/K86M, L72P/T79I, L70P/F92S, T79P, E35D/M47I/L65P/D90N, L25S/E35D/M47I/D90N, A71D, E81K/A91S, A12V/M47V/L70M, K34E/T41A/L72V, T41S/A71D/V84A, E35D/A71D, E35D/M47I, K36R/G78A, Q33E/T41A, M47V/N48H, M47L/V68A, S44P/A71D, Q27H/M43I/A71D/R73S, E35D/T57I/L70Q/A71D, M47I/E88D, M42I/I61V/A71D, P51A/A71D, H18Y/M47I/T57I/A71G, V20I/M47V/T57I/V84I, V20I/M47V/A71D, A71D/L72V/E95K, V22L/E35G/A71D/L72P, E35D/A71D, E35D/I67L/A71D, Q27H/E35G/A71D/L72P/T79I, T13R/M42V/M47I/A71D, E35D, E35D/M47I/L70M, E35D/A71D/L72V, E35D/M43L/L70M, A26P/E35D/M43I/L85Q/E88D, E35D/D46V/L85Q, Q27L/E35D/M47I/T57I/L70Q/E88D, M47V/I69F/A71D/V83I, E35D/T57A/A71D/L85Q, H18Y/A26T/E35D/A71D/L85Q, E35D/M47L, E23D/M42V/M43I/I58V/L70R, V68M/L70M/A71D/E95K, N55I/T157I69F, E35D/M43I/A71D, T41S/T57I/L70R, H18Y/A71D/L72P/E88V, V20I/A71D, E23G/A26S/E35D/T62N/A71D/L72V/L85M, A12T/E24D/E35D/D46V/I61V/L72P/E95V, V22L/E35D/M43L/A71G/D76H, E35G/K54E/A71D/L72P, L70Q/A71D, A26E/E35D/M47L/L85Q, D46E/A71D, Y31H/E35D/T41S/V68L/K93R/R94W, A26E/Q33R/E35D/M47L/L85Q/K86E, A26E/Q33R/E35D/M47L/L85Q, E35D/M47L/L85Q, A26E/Q33L/E35D/M47L/L85Q, A26E/Q33L/E35D/M47L, H18Y/A26E/Q33L/E35D/M47L/L85Q, Q33L/E35D/M47I, H18Y/Q33L/E35D/M47I, Q33L/E35D/D46E/M47I, Q33R/E35D/D46E/M47I, H18Y/E35D/M47L, Q33L/E35D/M47V, Q33L/E35D/M47V/T79A, Q33L/E35D/T41S/M47V, Q33L/E35D/M47I/L85Q, Q33L/E35D/M47I/T62N/L85Q, Q33L/E35D/M47V/L85Q, A26E/E35D/M43T/M47L/L85Q/R94Q, Q33R/E35D/K37E/M47V/L85Q, V22A/E23D/Q33L/E35D/M47V, E24D/Q33L/E35D/M47V/K54R/L85Q, S15P/Q33L/E35D/M47L/L85Q, E7D/E35D/M47I/L97Q, Q33L/E35D/T41S/M43I, E35D/M47I/K54R/L85E, Q33K/E35D/D46V/L85Q, Y31S/E35D/M47L/T79L/E88G, H18L/V22A/E35D/M47L/N48T/L85Q, Q27H/E35D/M47L/L85Q/R94Q/E95K, Q33K/E35D/M47V/K89E/K93R, E35D/M47I/E77A/L85Q/R94W, A26E/E35D/M43I/M47L/L85Q/K86E/R94W, Q27H/Q33L/E35D/M47V/N55D/L85Q/K89N, H18Y/V20A/Q33L/E35D/M47V/V53F, V22A/E35D/V68E/A71D, Q33L/E35D/M47L/A71G/F92S, V22A/R29H/E35D/D46E/M47I, Q33L/E35D/M43I/L85Q/R94W, H18Y/E35D/V68M/L97Q, Q33L/E35D/M47L/V68M/L85Q/E88D, Q33L/E35D/M43V/M47I/A71G, E35D/M47L/A71G/L97Q, E35D/M47V/A71G/L85M/L97Q, H18Y/Y31H/E35D/M47V/A71G/L85Q, E35D/D46E/M47V/L97Q, E35D/D46V/M47I/A71G/F92V, E35D/M47V/T62A/A71G/V83A/Y87H/L97M, Q33L/E35D/N48K/L85Q/L97Q, E35D/L85Q/K93T/E95V/L97Q, E35D/M47V/N48K/V68M/K89N, Q33L/E35D/M47I/N48D/A71G, R29H/E35D/M43V/M47I/I49V, Q27H/E35D/M47I/L85Q/D90G, E35D/M47I/L85Q/D90G, E35D/M47I/T62S/L85Q, A26E/E35D/M47L/A71G, E35D/M47I/Y87Q/K89E, V22A/E35D/M47I/Y87N, H18Y/A26E/E35D/M47L/L85Q/D90G, E35D/M47L/A71G/L85Q, E35D/M47V/A71G/E88D, E35D/A71G, E35D/M47V/A71G, I30V/E35D/M47V/A71G/A91V, I30V/V31C/E35D/M47V/A71G/L85M, V22D/E35D/M47L/L85Q, H18Y/E35D/N48K, E35D/T41S/M47V/A71G/K89N, E35D/M47V/N48T/L85Q, E35D/D46E/M47V/A71D/D90G, E35D/D46E/M47V/A71D, E35D/T41S/M43I/A71G/D90G, E35D/T41S/M43I/M47V/A71G, E35D/T41S/M43I/M47L/A71G, H18Y/Y22A/E35D/M47V/T62S/A71G, H18Y/A26E/E35D/M47L/V68M/A71G/D90G, E35D/K37E/M47V/N48D/L85Q/D90N, Q27H/E35D/D46V/M47L/A71G, V22L/Q27H/E35D/M47I/A71G, E35D/D46V/M47L/V68M/L85Q/E88D, E35D/T41S/M43V/M47I/L70M/A71G, E35D/D46E/M47V/N63D/L85Q, E35D/M47V/T62A/A71D/K93E, E35D/D46E/M47V/V68M/D90G/K93E, E35D/M43I/M47V/K89N, E35D/M47L/A71G/L85M/F92Y, E35D/M42V/M47V/E52D/L85Q, V22D/E35D/M47L/L70M/L97Q, E35D/T41S/M47V/L97Q, E35D/Y53H/A71G/D90G/L97R, E35D/A71D/L72V/R73H/E81K, Q33L/E35D/M43L/Y53F/T62S/L85Q, E35D/M38T/D46E/M47V/N48S, Q33R/E35D/M47V/N48K/L85M/F92L, E35D/M38T/M43V/M47V/N48R/L85Q, T28Y/Q33H/E35D/D46V/M47I/A71G, E35D/N48K/L72V, E35D/T41S/N48T, D46V/M47I/A71G, M47I/A71G, E35D/M43I/M47L/L85M, E35D/M43I/D46E/A71G/L85M, H18Y/E35D/M47L/A71G/A91S, E35D/M47I/N48K/I61F, E35D/M47V/T62S/L85Q, M43I/M47L/A71G, E35D/M47V, E35D/M47L/A71G/L85M, V22A/E35D/M47L/A71G, E35D/M47L/A71G, E35D/D46E/M47I, Q27H/E35D/M47I, E35D/D46E/L85M, E35D/D46E/A91G, E35D/D46E, E35D/L97R, H18Y/E35D, Q27L/E35D/M47V/I61V/L85M, E35D/M47V/I61V/L85M, E35D/M47V/L85M/R94Q, E35D/M47V/N48K/L85M, H18Y/E35D/M47V/N48K, A26E/Q27R/E35D/M47L/N48Y/L85Q, E35D/D46E/M47L/V68M/L85Q/F92L, E35D/M47I/T62S/L85Q/E88D, E24D/Q27R/E35D/T41S/M47V/L85Q, S15T/H18Y/E35D/M47V/T62A/N64S/A71G/L85Q/D90N, E35D/M47L/V68M/A71G/L85Q/D90G, H18Y/E35D/M47I/V68M/A71G/R94L, deltaE10-A98, Q33R/M47V/T62N/A71G, H18Y/Y22A/E35D/T41S/M47V/T62N/A71G/A91G, E35D/M47L/L70M, E35D/M47L/V68M, E35D/D46V/M47L/V68M/E88D, E35D/D46V/M47L/V68M/D90G, E35D/D46V/M47L/V68M/K89N, E35D/D46V/M47L/V68M/L85Q, E35D/D46V/M47L/V68M, E35D/D46V/M47L/V70M, E35D/D46V/M47L/V70M/L85Q, E35D/M47V/N48K/V68M, E24D/E35D/M47L/V68M/E95V/L97Q, E35D/D46E/M47I/T62A/V68M/L85M/Y87C, E35D/D46E/M47I/V68M/L85M, E35D/D46E/M47L/V68M/A71G/Y87C/K93R, E35D/D46E/M47L/V68M/T79M/L85M, E35D/D46E/M47L/V68M/T79M/L85M/L97Q, E35D/D46E/M47V/V68M/L85Q, E35D/M43I/M47L/V68M, E35D/M47I/V68M/Y87N, E35D/M47L/V68M/E95V/L97Q, E35D/M47L/Y53F/V68M/A71G/K93R/E95V, E35D/M47V/N48K/V68M/A71G/L85M, E35D/M47V/N48K/V68M/L85M, E35D/M47V/V68M/L85M, E35D/M47V/V68M/L85M/Y87D, E35D/T41S/D46E/M47I/V68M/K93R/E95V, H18Y/E35D/D46E/M47I/V68M/R94L, H18Y/E35D/M38I/M47L/V68M/L85M, H18Y/E35D/M47I/V68M/Y87N, H18Y/E35D/M47L/V68M/A71G/L85M, H18Y/E35D/M47L/V68M/E95V/L97Q, H18Y/E35D/M47L/Y53F/V68M/A71G, H18Y/E35D/M47L/Y53F/V68M/A71G/K93R/E95V, H18Y/E35D/M47V/V68M/L85M, H18Y/E35D/V68M/A71G/R94Q/E95V, H18Y/E35D/V68M/L85M/R94Q, H18Y/E35D/V68M/T79M/L85M, H18Y/Y22D/E35D/M47V/N48K/V68M, Q27L/Q33L/E35D/T41S/M47V/N48K/V68M/L85M, Q33L/E35D/M47V/T62S/V68M/L85M, Q33R/E35D/M38I/M47L/V68M, R29C/E35D/M47L/V68M/A71G/L85M, S21P/E35D/K37E/D46E/M47I/V68M, S21P/E35D/K37E/D46E/M47I/V68M/R94L, T13R/E35D/M47L/V68M, T13R/H18Y/E35D/V68M/L85M/R94Q, T13R/Q27L/Q33L/E35D/T41S/M47V/N48K/V68M/L85M, T13R/Q33L/E35D/M47L/V68M/L85M, T13R/Q33L/E35D/M47V/T62S/V68M/L85M, T13R/Q33R/E35D/M38I/M47L/V68M, T13R/Q33R/E35D/M38I/M47L/V68M/E95V/L97Q, T13R/Q33R/E35D/M38I/M47L/V68M/L85M, T13R/Q33R/E35D/M38I/M47L/V68M/L85M/R94Q, T13R/Q33R/E35D/M47L/V68M, T13R/Q33R/E35D/M47L/V68M/L85M, V22D/E24D/E35D/M47L/V68M, V22D/E24D/E35D/M47L/V68M/L85M/D90G, V22D/E24D/E35D/M47V/V68M, D46V, M47L, V68M, L85Q, E35D/D46V, E35D/V68M, E35D/L85Q, D46V/M47L, D46V/V68M, D46V/L85Q, M47L/V68M, M47L/L85Q, V68M/L85Q, E35D/D46V/M47L, E35D/D46V/V68M, E35D/D46V/L85Q, E35D/V68M/L85Q, D46V/M47L/V68M, D46V/M47L/L85Q, D46V/V68M/L85Q, M47L/V68M/L85Q, E35D/D46V/M47L/L85Q, E35D/D46V/V68M/L85Q, E35D/M47L/V68M/L85Q, D46V/M47L/V68M/L85Q, M47V, N48K, K89N, E35D/N48K, E35D/K89N, M47V/N48K, M47V/V68M, M47V/K89N, N48K/V68M, N48K/K89N, V68M/K89N, E35D/M47V/N48K, E35D/M47V/V68M, E35D/M47V/K89N, E35D/N48K/V68M, E35D/N48K/K89N, E35D/V68M/K89N, M47V/N48K/V68M, M47V/N48K/K89N, M47V/V68M/K89N, N48K/V68M/K89N, E35D/M47V/N48K/K89N, E35D/M47V/V68M/K89N, E35D/N48K/V68M/K89N, M47V/N48K/V68M/K89N, E35D/D46V/M47V/N48K/V68M, E35D/D46V/M47V/V68M/L85Q, E35D/D46V/M47V/V68M/K89N, E35D/M47V/N48K/V68M/L85Q, E35D/M47V/V68M/L85Q/K89N, A26E/E35D/M47L/V68M/A71G/D90G, H18Y/E35D/M47L/V68M/A71G/D90G, H18Y/A26E/M47L/V68M/A71G/D90G, H18Y/A26E/E35D/V68M/A71G/D90G, H18Y/A26E/E35D/M47L/A71G/D90G, H18Y/A26E/E35D/M47L/V68M/D90G, H18Y/A26E/E35D/M47L/V68M/A71G, E35D/M47L/V68M/A71G/D90G, H18Y/M47L/V68M/A71G/D90G, H18Y/A26E/V68M/A71G/D90G, H18Y/A26E/E35D/A71G/D90G, H18Y/A26E/E35D/M47L/D90G, H18Y/A26E/E35D/M47L/V68M, A26E/M47L/V68M/A71G/D90G, A26E/E35D/V68M/A71G/D90G, A26E/E35D/M47L/A71G/D90G, A26E/E35D/M47L/V68M/D90G, A26E/E35D/M47L/V68M/A71G, H18Y/E35D/V68M/A71G/D90G, H18Y/E35D/M47L/A71G/D90G, H18Y/E35D/M47L/V68M/D90G, H18Y/E35D/M47L/V68M/A71G, H18Y/A26E/M47L/A71G/D90G, H18Y/A26E/M47L/V68M/D90G, H18Y/A26E/M47L/V68M/A71G, H18Y/A26E/E35D/V68M/D90G, H18Y/A26E/E35D/V68M/A71G, H18Y/A26E/E35D/M47L/A71G, M47L/V68M/A71G/D90G, H18Y/V68M/A71G/D90G, H18Y/A26E/A71G/D90G, H18Y/A26E/E35D/D90G, H18Y/A26E/E35D/M47L, E35D/V68M/A71G/D90G, E35D/M47L/A71G/D90G, E35D/M47L/V68M/D90G, E35D/M47L/V68M/A71G, A26E/V68M/A71G/D90G, A26E/M47L/A71G/D90G, A26E/M47L/V68M/D90G, A26E/M47L/V68M/A71G, A26E/E35D/A71G/D90G, A26E/E35D/V68M/D90G, A26E/E35D/V68M/A71G, A26E/E35D/M47L/D90G, A26E/E35D/M47L/V68M, H18Y/M47L/A71G/D90G, H18Y/M47L/V68M/D90G, H18Y/M47L/V68M/A71G, H18Y/E35D/A71G/D90G, H18Y/E35D/V68M/D90G, H18Y/E35D/V68M/A71G, H18Y/E35D/M47L/D90G, H18Y/E35D/M47L/A71G, H18Y/E35D/M47L/V68M, H18Y/A26E/V68M/D90G, H18Y/A26E/V68M/A71G, H18Y/A26E/M47L/D90G, H18Y/A26E/M47L/A71G, H18Y/A26E/M47L/V68M, H18Y/A26E/E35D/A71G, H18Y/A26E/E35D/V68M, H18Y/E35D/M47V/V68M/A71G, H18C/A26P/E35D/M47L/V68M/A71G, H18I/A26P/E35D/M47V/V68M/A71G, H18L/A26N/D46E/V68M/A71G/D90G, H18L/E35D/M47V/V68M/A71G/D90G, H18T/A26N/E35D/M47L/V68M/A71G, H18V/A26K/E35D/M47L/V68M/A71G, H18V/A26N/E35D/M47V/V68M/A71G, H18V/A26P/E35D/M47V/V68L/A71G, H18V/A26P/E35D/M47L/V68M/A71G, H18V/E35D/M47V/V68M/A71G/D90G, H18Y/A26P/E35D/M47U/V68M/A71G, H18Y/A26P/E35D/M47V/V68M/A71G, H18Y/E35D/M47V/V68L/A71G/D90G, H18Y/E35D/M47V/V68M/A71G/D90G, A26P/E35D/M47I/V68M/A71G/D90G, H18V/A26G/E35D/M47V/V68M/A71G/D90G, H18V/A26S/E35D/M47L/V68M/A71G/D90G, H18V/A26R/E35D/M47L/V68M/A71G/D90G, H18V/A26D/E35D/M47V/V68M/A71G/D90G, H18V/A26Q/E35D/M47V/V68L/A71G/D90G, H18A/A26P/E35D/M47L/V68M/A71G/D90G, H18A/A26N/E35D/M47L/V68M/A71G/D90G, H18F/A26P/E35D/M47I/V68M/A71G/D90G, H18F/A26H/E35D/M47L/V68M/A71G/D90G, H18F/A26N/E35D/M47V/V68M/A71G/D90K, H18Y/A26N/E35D/M47F/V68M/A71G/D90G, H18Y/A26P/E35D/M47V/V68U/A71G/D90G, H18Y/A26Q/E35D/M47T/V68M/A71G/D90G, H18R/A26P/E35D/D46N/M47V/V68M/A71G/D90P, H18F/A26D/E35D/D46E/M47T/V68M/A71G/D90G, L70Q/A91G, L70Q/A91G/T130A, L70Q/A91G/I118A/T120S/T130A, V4M/L70Q/A91G/T120S/T130A, L70Q/A91G/T120S/T130A, V20L/L70Q/A91S/T120S/T130A, S44P/L70Q/A91G/T130A, L70Q/A91G/E117G/T120S/T130A, A91G/T120S/T130A, L70R/A91G/T120S/T130A, L70Q/E81A/A91G/T120S/I127T/T130A, L70Q/Y87N/A91G/T130A, T28S/L70Q/A91G/E95K/T120S/T130A, N63S/L70Q/A91G/T120S/T130A, K36E/167T/L70Q/A91G/T120S/T130A/N152T, E52G/L70Q/A91G/T120S/T130A, K37E/F59S/L70Q/A91G/T120S/T130A, A91G/S103P, K89E/T130A, D60V/A91G/T120S/T130A, K54M/A91G/T120S, M38T/L70Q/E77G/A91G/T120S/T130A/N152T, R29H/E52G/L70R/E88G/A91G/T130A, Y31H/T41G/L70Q/A91G/T120S/T130A, V68A/T110A, S66H/D90G/T110A/F116L, R29H/E52G/T120S/T130A, A91G/L102S, I67T/L70Q/A91G/T120S, L70Q/A91G/T110A/T120S/T130A, M38V/T41D/M43I/W50G/D76G/V83A/K89E/T120S/T130A, V22A/L70Q/S121P, A12V/S15F/Y31H/T41G/T130A/P137L/N152T, I67F/L70R/E88G/A91G/T120S/T130A, E24G/L25P/L70Q/T120S, A91G/F92L/F108L/T120S, R29D/Y31L/Q33H/K36G/M38I/T41A/M43R/M47T/E81V/L85R/K89N/A91T/F92P/K93V/R94L/I118T/N149S, R29D/Y31L/Q33H/K36G/M38I/T41A/M43R/M47T/E81V/L85R/K89N/A91T/F92P/K93V/R94L/N144S/N149S, R29D/Y31L/Q33H/K36G/M38I/T41A/M42T/M43R/M47T/E81V/L85R/K89N/A91T/F92P/K93V/R94L/L148S/N149S, E24G/R29D/Y31L/Q33H/K36G/M38I/T41A/M43R/M47T/F59L/E81V/L85R/K89N/A91T/F92P/K93V/R94L/H96R/N149S/C182S, R29D/Y31L/Q33H/K36G/M38I/T41A/M43R/M47T/E81V/L85R/K89N/A91T/F92P/K93V/R94L/N149S, R29V/M43Q/E81R/L85I/K89R/D90L/A91E/F92N/K93Q/R94G, T41I/A91G, K89R/D90K/A91G/F92Y/K93R/N122S/N177S, K89R/D90K/A91G/F92Y/K93R, K36G/K37Q/M38I/F59L/E81V/L85R/K89N/A91T/F92P/K93V/R94L/E 99G/T130A/N149S, E88D/K89R/D90K/A91G/F92Y/K93R, K36G/K37Q/M38I/L40M, R29H/Y31H/T41G/Y87N/E88G/K89E/D90N/A91G/P109S, A12T/H18L/M43V/F59L/E77K/P109S/I118T, R29V/Y31F/K36G/M38L/M43Q/E81R/V83I/L85I/K89R/D90L/A91E/F92N/K93Q/R94G, V68M/L70P/L72P/K86E, R29D/Y31L/Q33H/K36G/M38I/T41A/M43R/M47T/E81V/L85R/K89N/A91T/F92P/K93V/R94L/T120S/I127T/T130A/K169E, R29D/Y31L/Q33H/K36G/M38I/T41A/M43R/M47T/L70Q/E81V/L85R/K89N/A91 T/F92P/K93V/R94L/T120S/I127T/T130A, H18L/R29D/Y31L/Q33H/K36G/M38I/T41A/M43R/M47T/E81V/L85R/K89N/A91T/F92P/K93V/R94L/T120S/I127T/T130A/K169E, R29D/Y31L/Q33H/K36G/M38I/T41A/M43R/M47T/E81V/L85R/K89N/A91T/F92P/K93V/R94L/T120S/T130A/M174T, R29D/Y31L/Q33H/K36G/M38I/T41A/M43R/M47T/F59L/E81V/L85R/K89N/A91T/F92P/K93V/R94L/T120S/I127T/T130A/H188D, H18R/R29D/Y31L/Q33H/K36G/K37E/M38I/T41A/M43R/M47T/L70Q/E81V/L85R/K89N/A91T/F92P/K93V/R94L/T120S/T130A/K169E/H188D, R29D/Y31L/Q33H/K36G/M38I/T41A/M43R/M 47T/L70Q/E81V/L85R/K89N/A91T/F92P/K93V/R94L/T120S/I127T/T130A/E143G/K169E/M174V/H188D, R29D/Y31L/Q33H/K36G/M38I/T41A/M43R/M47T/L70Q/E81V/L85R/K89N/A91T/F92P/K93V/R 94L/T120S/I127T/T130A, R29D/Y31L/Q33H/K36G/M38I/T41A/M43R/M47T/E81V/L85R/K89N/A91T/F92P/K93V/R94L/T120S/I127T/T130A/H188D, R29D/Y31L/Q33H/K36G/M38I/T41A/M43R/M47T/L70Q/E81V/L85R/K89N/A91T/F92P/K93V/R94L/T120S/I127T/T130A/K169E, R29D/Y31L/Q33H/K36G/M38I/T41A/M43R/M47T/L70Q/E81V/L85R/K89N/A91T/F92P/K93V/R94L/T120S/I127T/T130A, R29D/Y31L/Q33H/K36G/M38I/T41A/M43R/M47T/L85R/K89N/A91T/F92P/K93V/R94L/T120S/I127T/T130A/K169E/H188D, R29D/V31L/Q33H/K36G/M38I/T41A/M43R/M47T/E81V/L85R/K89N/A91T/F92P/K93V/R94L/F108L/T120S/T130A/K169E/H188D, R29D/Y31L/Q33H/K36G/M38I/T741A/M43R/M47T/L70Q/E81V/L85R/K89N/A91T/F92P/K93V/R94L/T130A/H188D, R29D/Y31L/Q33H/K36G/M38I/T41A/M43R/M47T/L70Q/E81V/L85R/K89N/A91T/F92P/K93V/R94L/T120S/I127T/T130A/K169E, H18L/R29D/V31L/Q33H/K36G/M38I/T41A/M43R/M47T/L70Q/E81V/L85R/K89N/A91T/F92P/K93V/R94L/T120S/T130A/K169E/H188D, R29D/V31L/Q33H/K36G/M38I/T41A/M43R/M47T/E81V/L85R/K 89N/A91T/F92P/K93V/R94I 120S/I127T/C128Y/T130A/H188D, R29D/V31L/Q33H/K36G/M38I/T41A/M43R/M47T/E81V/L85R/K89N/A91T/F92P/K93V/R94F/T130A/K169E, H18L/R29D/V31L/Q33H/K 36G/M38I/T41A/M43R/M47T/E81V/L85R/K89N/A91T/F92P/K93V/R94L/T 130A, H18L/R29D/V31L/Q 33H/K36G/M38I/T41A/M43R/M47T/L70Q/E81V/L85R/K89N/A91T/F92P/K93V/R94L/T120S/T130A/K169E, R29D/V31L/Q33H/K36G/M38I/T41A/M43R/M47T/L70Q/E81V/L85R/K89N/A91T/F92P/K93I/R94L/L97R/T130A, R29D/V31L/Q33H/K36G/M38I/T41A/M43R/M47T/L70Q/E81V/L85R/K89N/A91T/F92P/K93I/R94L/L97R/T130A/L148S, H18L/R29D/Y31L/Q33H/K36G/M38I/T41A/M43R/M47T/E81V/L85R/K89N/A91T/F92P/K93V/R94L/I118V/T120S/I127T/T130A/K169E, R29D/V31L/Q33H/K36G/M38I/T41A/M43R/M47T/I61N/E81V/L85R/K89N/A91T/F92P/K93V/R94F/V104A/T120S/T130A, R29D/Y 31L/Q33H/K36G/M38I/T41A/M43R/M47T/E81V/L85R/K89N/F92P/K93V/R94F/I118V/T130A, R29D/Y31L/Q33H/K36G/M38I/T41A/M43R/M47T/T62S/E81V/L85R/K89N/A91T/F92P/K93V/R94L/I118V/T120S/T130A/K169E/T175A, H18L/R29D/V31L/Q33H/K36G/M38I/T41A/M43R/M47T/E81V/L85R/K 89N/A91T/F92P/K93V/R94L/F116S/T130A/H188D, H18L/R29D/Y31L/Q33H/K36G/M38I/T41A/M43R/M47T/E81V/L85R/K89N/A91T/F92P/K93V/R94I 120S/I127T/T130A/L142S/H188D, C16S/H18L/R29D/V31L/Q33H/K36G/M38I/T41A/M43R/M47T/E81V/L85R/K89N/A91T/F92P/K93V/R94L/T110A/H188D, R29D/V31L/Q33H/K36G/M38I/T41A/M43R/M47T/A91G/T120S/I127T/T130A/H188D, R29D/V31L/Q33H/K36G/M38I/T41A/M43R/M47T/L70Q/D76G/A91G/S103L/T120S/I127T/T130A, DELTAQ33N53C/L85R/K89N/A91T/F92P/K93V/R94I/T120S/I127T/T130A/K169E, T62S/E81V/L85R/K89N/A91T/F92P/K93V/R94L/T120S/T130A/K169E, R29D/V31L/Q33H/K36G/M38 I/T41A/M43R/M47T/E81V/L85R/K89N/A91T/F92P/K93V/R94L/S129L/H188D, K9E/E10R/V11S/A12 G/T13N/K14A/S15V/C16L/G17W/H18Y/Y53C/L70Q/D90G/T130A/N149D/N152T/H188D, H18L/R29D/V31L/Q33H/K36G/T41A/M43R/M47T/E81V/L85R/K89N/A91T/F92P/K93V/R94L/I118V/T120S/I127T/T130A/H188D, K89E/K93E/T130A, S21P/R29D/V31L/Q33H/K36G/M38I/T41A/M43R/M47T/N48I/V68A/E81V/L85R/K89N/A91T/F92P/K 93V/R94L/P109H/I126L/K169I, H18L/R29D/V31L/Q33H/K36G/M38I/T41A/M43R/M47T/P74L/Y80N/E81V/L85R/K89N/A91T/F92P/K93V/R94L/L97R, S21P/R29D/V31L/Q33H/K36G/M38I/T41A/M43R/M 47T/P74L/Y80N/E81V/L85R/K89N/D90N/A91T/F92P/K93V/R94L/T130A/N149S/E162G, H18L/R29D/V31L/Q33H/K36G/M38I/T41A/M43R/M47T/V68M/E81V/L85R/K89N/A91T/F92P/K93V/R94L/T1730A, R29D/V31L/Q33H/K36G/M38I/T41A/M43R/M47T/V68M/E81V/L85R/K89N/A91T/F92P/K93V/R94L/T130A/N149S/R190S, H18L/R29D/V31L/Q33H/K36G/M38I/T41A/M43R/M47T/P74L/Y80N/E81V/L85R/K89N/A91T/F92P/K93V/R94L/T130A/R190S, C16G/V22A/R29D/V31L/Q33H/K36G/M38I/T41A/M43R/M47T/V68M/D76G/E81V/L85R/K89N/A91T/F92P/K93V/R94L/I118T/T130A/S140 T/N149S/K169I/H178R/N192D, R29D/V31L/Q33H/K36G/M38I/T41A/M43R/M47T/E81V/L85R/K89N/A91T/F92P/K93V/R94F/E117V/I118T/N149S/S168G/H188Q, V22A/R29D/V31L/Q33H/K36G/M38I/T41A/M43R/M47T/V68M/E81V/L85R/K89N/A91T/F92P/K93V/R94L/T130A, R29D/V31L/Q33H/K36G/M38I/T41A/M43R/M47T/N64S/E81V/L85R/K89N/A91T/F92P/K93V/R94F/I 118T/T130A/N149S/K169I, V22A/R29D/V31L/Q33H/K36G/M38I/T41A/M43R/M47T/V68M/E81V/L85R/K89N/A91T/F92P/K93V/R94L/D115G/I118T/T130A/G133D/N149S, S129P, A91G/S129P, I69T/L70Q/A91G/T120S, Y31H/S129P, T28A/R29D/V31L/Q33H/K36G/M38I/T41A/M43R/M47T/E81V/L85R/K89N/A91T/F92P/K93V/R94L/V104L/T130A/N149S, H18L/R29D/V31L/Q33H/K36G/M38I/T41A/M43R/M47T/E81V/L85R/K89N/A91T/F92P/K93V/R94L/L97R/N149S/H188Q, H18L/R29D/V31L/Q33H/K36G/M38I/T41A/M43R/M47T/E 81V/L85R/K89N/A91T/F92P/K93V/R94L/L97R/N149S, H18L/R29D/V31L/Q33H/K36G/M38I/T41A/M 43R/M47T/V68A/E81V/L85R/K89N/A91T/F92P/K93V/R94L/T 130A/N149S/T154I, A12G/R29D/Y31L/Q33H/K36G/M38I/T41A/M43R/M47T/V68A/E81V/L85R/K89N/A91T/F92P/K93V/R94L/L97R/T130A/L183H, R29D/V31L/Q33H/K36G/M38I/T41A/M43R/M47T/E81V/L85R/K89N/A91T/F92P/K93V/R94L/I118T/T130A/S140T/N149S/K169S, R29D/V31L/Q33H/K36G/M38I/T41A/M43R/M47T/E81V/L85R/K89N/A91T/F92P/K93V/R94L/I118T/T130A/N149S/K169I/Q193L, V22A/R29D/V31L/Q33H/K36G/M38I/T41A/M43R/M47T/E81V/L85R/K89N/A91T/F92P/K93V/R94L/I118T/T130A/N149S, R29D/V31L/Q33H/K36G/M38I/T41A/M43R/M47T/E81V/L85R/K89N/A91T/F92P/K93V/R94L/I118T/T130A/N149S, R29D/V31L/Q33H/K36G/M38I/T41A/M43R/M47T/E81V/L85R/K89N/A91T/F92P/K93V/R94L/I118T/T130A/N149S/K169I, R29D/V31L/Q33H/K36G/M38I/T41A/M43R/M47T/E81V/L85R/K 89N/A91T/F92P/K93V/R94L/T130A/N149S/K169I, I118T/C128R, Q27R/R29C/M42T/S129P/E160G, S129P/T154A, S21P/L70Q/D90G/T120S/T130A, L70Q/A91G/N144D, L70Q/A91G/I118A/T120S/T130A/K169E, V4M/L70Q/A91G/I118V/T120S/T130A/K169E, L70Q/A91G/I118V/T120S/T130A/K169E, L70Q/A91G/I118V/T120S/T130A, V20L/L70Q/A91S/I118V/T120S/T130A, L70Q/A91G/E117G/I118V/T120S/T130A, A91G/I118V/T120S/T130A, L70R/A91G/I118V/T120S/T130A/T199S, L70Q/E81A/A91G/I118V/T120S/I127T/T130A, T28S/L70Q/A91G/E95K/I118V/T120S/I126V/T130A/K169E, N63S/L70Q/A91G/S 114T/I118V/T120S/T130A, K36E/I67T/L70Q/A91G/I118V/T120S/T130A/N152T, E52G/L70Q/A91G/D107N/I118V/T120S/T130A/K169E, K37E/F59S/L70Q/A91G/I118V/T120S/T130A/K185E, D60V/A91G/I118V/T120S/T130A/K169E, K54M/L70Q/A91G/Y164H/T120S, M38T/L70Q/E77G/A91G/I118V/T120S/T130A/N152T, Y31H/T41G/M43L/L70Q/A91G/I118V/T120S/I126V/T130A, L65H/D90G/T110A/F116L, R29H/E52G/D90N/I118V/T120S/T130A, I67T/L70Q/A91G/I118V/T120S, L70Q/A91G/T110A/I118V/T120S/T130A, M38V/T41D/M43I/W50G/D76G/V83A/K89E/I118V/T120S/I126V/I 130A, A12V/S15F/Y31H/M38I/T41G/M43L/D90N/T130A/P137L/N149D/N152T, I67F/L70R/E88G/A91G/I118V/T120S/T130A, E24G/L25P/L70Q/A91G/I118V/T120S/N152T, A91G/F92L/F108L/I118V/T120S, E88D/K89R/D90K/A91G/F92Y/K93R/N122S/N177S, K36G/K37Q/M38I/L40M/F59L/E81V/L85R/K89N/A91T/F92P/K93V/R94L/E99G/T130A/N149S, K36G/L40M, R29D/Y31L/Q33H/K36G/M38I/T41A/M43R/M47T/E81V/L85R/K89N/A91T/F92P/K93V/R94L/I118V/T120S/I127T/T130A/K169E, R29D/Y31L/Q33H/K36G/M38I/T41A/M43R/M47T/L70Q/E81V/L85R/K89N/A91T/F92P/K93V/R94L/I118V/T120S/I127T/T130A, H18L/R29D/Y31L/Q33H/K36G/M38I/T41A/M43R/M47T/E81V/L85R/K89N/A91T/F92P/K93V/R94L/I118V/T120S/T127T/T7130A/K169E, R29D/Y31L/Q33H/K36G/M38I/T41A/M43R/M47T/E81V/L85R/K89N/A91T/F92P/K93V/R94L/I118V/T120S/T130A/K169E/M174T, R29D/Y31L/Q33H/K36G/M38I/T41A/M43R/M47T/N48D/F59L/E81V/L85R/K89N/A91T/F92P/K93V/R94L/I118V/T120S/I127T/T130A/H188D, H18R/R29D/Y31L/Q33H/K36G/K37E/M38I/T41A/M43R/M47T/L70Q/E81V/L85R/K89N/A91T/F92P/K93V/R94L/I118V/T120S/T130A/K169E/H188D, R29D/Y31L/Q33H/K36G/M38I/T41A/M43R/M47T/L 70Q/E81V/L85R/K89N/A91T/F92P/K93V/R94L/I118V/T120S/I127T/T130A/E143G/K169E/M174V/H188D, R29D/I30V/Y31L/Q33H/K36G/M38I/T41A/M43R/M47T/E81V/L85R/K89N/A91T/F92P/K93V/R94L/I118V/T120S/I127T/T130A/H188D, R29D/Y31L/Q33H/K36G/M38I/T41A/M43R/M47T/E81V/L85R/K89N/A91T/F92P/K93V/R94L/I118V/T120S/I27T/T130A/H188D, R29D/Y31L/Q33H/K36G/M38I/T41A/M43R/M47T/L70Q/E81V/L85R/K89N/A91T/F92P/K93V/R94L/I 118V/T120S/I127T/T130A/K169E, R29D/Y31L/Q33H/K36G/M38I/T41A/M43R/M47T/L70Q/E81V/K89N/A91T/F92P/K93V/R94L/I118V/T120S/I127T/T130A, R29D/Y31L/Q33H/K36G/M38I/T41A/M43R/M47T/L85R/K89N/A91T/F92P/K93V/R94L/I118V/T120S/I127T/T130A/K169E/H188D, R29D/Y31L/Q33H/K36G/M38I/T41A/M43R/M47T/E81V/L85R/K89N/A91T/F92P/K93V/R94L/F108U I118V/T120S/T130A/K169E/H188D, R29D/Y31L/Q33H/K36G/M38I/T41A/M43R/M47T/L70Q/E81V/L 85R/K89N/A91T/F92P/K93V/R94L/I118V/T120S/T130A/N149D/K169E/H188D, H18L/R29D/Y31L/Q33H/K36G/M38I/T41A/M43R/M47T/L70Q/E81V/L85R/K89N/A91T/F92P/K93V/R94L/I118V/T120S/T130A/K169E/H188D, R29D/Y31L/Q33H/K36G/M38I/T741A/M43R/M47T/E81V/L 85R/K89N/A91T/F92P/K93V/R94L/I118V/T120S/I127T/C128Y/T130A/H188D, H18L/R29D/Y31L/Q33H/K36G/M38I/T41A/M43R/M47T/E81V/L85R/K89N/A91T/F92P/K93V/R94L/E99D/T130A, H18L/R29D/V31L/Q33H/K36G/M38I/T41A/M43R/M47T/L70Q/E81V/L85R/K89N/A91T/F92P/K93V/R94L/I118V/T120S/T130A/K169E, R29D/Y31L/Q33H/K36G/M38I/T41A/M43R/M47T/I61N/E81V/L85R/K89N/A91T/F92P/K93V/R94F/V104A/I118V/T120S/I126V/T130A, R29D/Y31L/Q33H/K36G/M38I/T41A/M43R/M47T/E81V/L85R/K89N/A91T/F92P/K93V/R94F/I118V/T120S/T130A, R29D/Y31L/Q33H/K36G/M38V/T41A/M43R/M47T/T62S/E81V/L85R/K89N/A91T/F92P/K93V/R94L/I118V/T120S/T17130A/K169E/T175A, H18L/R29D/Y31L/Q33H/K36G/M38I/T41A/M43R/M47T/E81V/L85R/K89N/A91T/F92P/K93V/R94L/I118V/T120S/I127T/T130A/L142S/H188D, C16S/H118L/R29D/Y31L/Q33H/K36G/M38I/T41A/M43R/M47T/E81V/L85R/K89N/A91T/F92P/K93V/R94I 1710A/I118V/H188D, R29D/Y31L/Q33H/K36G/M38I/T41A/M43R/M47T/A91G/I118V/T7120S/I127T/1730A/H188D, R29D/Y31L/Q33H/K36G/M38I/T41A/M43R/M47T/L70Q/D76G/A91G/S103L/I118V/T120S/I127T/T130A, Y53C/L85R/K89N/A91T/F92P/K93V/R94L/I118V/T120S/I127T/T130A/K169, T62S/E81V/L85R/K89N/A91T/F92P/K93V/R94L/I118V/T120S/T130A/K169E, Y53C/L70Q/D90G/T130A/N149D/N152T/H188D, H18L/R29D/Y31L/Q33H/K36G/M38I/T41A/M43R/M47T/E81V/L85R/K89N/A91T/F92P/K93V/R94L/I118V/T120S/I127T/T130A/H188D, H18L/R29D/Y31L/Q33H/K36G/M38I/T41A/M43R/M47T/E81V/L85R/K89N/A91T/F92P/K93V/R94L/T130A/N149SS21P/L70Q/D90G/I118V/T120S/T130A, I67T/L70Q/A91G/I118V/T120S/T130A.

In some embodiments, the variant CD80 polypeptide has one or more amino acid modifications (e.g., substitutions) in an unmodified CD80 or specific binding fragment there of corresponding to position(s) 7, 23, 26, 34, 49, 51, 55, 57, 58, 71, 73, 78, 79, 82, and/or 84, with reference to numbering of SEQ ID NO: 2. In some embodiments, the variant CD80 polypeptide has one or more amino acid modifications (e.g., substitutions) in an unmodified CD80 or specific binding fragment there of corresponding to position(s) 7, 23, 26, 34, 49, 51, 55, 57, 58, 71, 73, 78, 79, 82, or 84 with reference to numbering of SEQ ID NO: 2. In some embodiments, the variant CD80 polypeptide has a modification, e.g., amino acid substitution, at any 2 or more of the foregoing positions, such as 2, 3, 4, 5, 6, 7, 8, 9, 10, 11, 12, 13, 14, 15 or more of the positions.

In some embodiments, the variant CD80 polypeptide has one or more amino acid substitution selected from among E7D, T13A, T13R, L14A, S15P, S15T, C16R, H18A, H18C, H18F, H18I, H18T, H18V, H18Y, V20A, V20I, V22D, V22I, V22L, E23D, E23G, E24D, L25S, A26D, A26E, A26G, A26H, A26K, A26N, A26P, A26Q, A26R, A26S, A26T, Q27H, Q27L, T28Y, I30F, I30T, Y31C, Y31S, Q33E, Q33K, Q33L, Q33R, K34E, E35D, E35G, K36R, T41S, M42I, M42V, M43T, D46E, D46N, D46V, M47F, M47I, M47L, M47V, M47Y, N48H, N48K, N48R, N48T, N48Y, I49V, P51A, E52D, Y53F, Y53H, K54E, K54N, K54R, N55D, N55I, T57A, T57I, I58V, I61F, I61V, T62A, T62N, N63D, L65P, I67L, I67V, V68E, V68I, V68L, I69F, L70M, A71D, A71G, L72V, R73H, R73S, P74S, D76H, E77A, G78A, T79A, T79I, T79L, T79M, T79P, E81G, E81K, C82R, V84A, V84I, L85M, L85Q, K86M, Y87C, Y87D, Y87H, Y87Q, E88V, D90P, A91V, F92S, F92V, K93T, R94Q, R94W, E95D, E95V, L97M, L97Q, and K169S.

In some embodiments, the variant CD80 polypeptide comprises the amino acid modifications L70P, I30F/L70P, Q27H41S/A71D, I30T/L70R, T13R/C16R/L70Q/A71D, T57I, M43I/C82R, V22L/M38V/M47T/A71D/L85M, I30V/T57I/L70P/A71D/A91T, V22I/L70M/A71D, N55D/L70P/E77G, T57A/I69T, N55D/K86M, L72P/T79I, L70P/F92S, T79P, E35D/M47I/L65P/D90N, L25S/E35D/M47I/D90N, A71D, T13A/I61N/A71D, E81K/A91S, A12V/M47V/L70M, K34E/T41A/L72V, T41S/A71D/V84A, E35D/A71D, E35D/M47I, K36R/G78A, Q33E/T41A, M47V/N48H, M47L/V68A, S44P/A71D, Q27H/M43I/A71D/R73S, E35D/T57I/L70Q/A71D, M47I/E88D, M42I/I61V/A71D, P51A/A71D, H18Y/M47I/T57I/A71G, V20I/M47V/T57I/V84I, V20I/M47V/A71D, A71D/L72V/E95K, V22L/E35G/A71D/L72P, E35D/A71D, E35D/I67L/A71D, Q27H/E35G/A71D/L72P/T79I, T13R/M42V/M47/A71D, E35D, E35D/M47I/L70M, E35D/A71D/L72V, E35D/M43L/L70M, A26P/E35D/M43I/L85Q/E88D, E35D/D46V/L85Q, Q27L/E35D/M47I/T57I/L70Q/E88D, M47V/I69F/A71D/V83I, E35D/T57A/A71D/L85Q, H18Y/A26T/E35D/A71D/L85Q, E35D/M47L, E23D/M42V/M43I/I58V/L70R, V68M/L70M/A71D/E95K, N55I/T57I/I69F, E35D/M43I/A71D, T41S/T57I/L70R, H18Y/A71D/L72P/E88V, V20I/A71D, E23G/A26S/E35D/T62N/A71D/L72V/L85M, A12T/E24D/E35D/D46V/I61V/L72P/E95V, V22L/E35D/M43L/A71G/D76H, E35G/K54E/A71D/L72P, L70Q/A71D, A26E/E35D/M47L/L85Q, D46E/A71D, Y31H/E35D/T41S/V68L/K93R/R94W, A26E/Q33R/E35D/M47L/L85Q/K86E, A26E/Q33R/E35D/M47L/L85Q, E35D/M47L/L85Q, A26E/Q33L/E35D/M47L/L85Q, A26E/Q33L/E35D/M47L, H18Y/A26E/Q33L/E35D/M47L/L85Q, Q33L/E35D/M47I, H18Y/Q33L/E35D/M47I, Q33L/E35D/D46E/M47I, Q33R/E35D/D46E/M47I, H18Y/E35D/M47L, Q33L/E35D/M47V, Q33L/E35D/M47V/T79A, Q33L/E35D/T41S/M47V, Q33L/E35D/M47I/L85Q, Q33L/E35D/M47I/T62N/L85Q, Q33L/E35D/M47V/L85Q, A26E/E35D/M43T/M47L/L85Q/R94Q, Q33R/E35D/K37E/M47V/L85Q, V22A/E23D/Q33L/E35D/M47V, E24D/Q33L/E35D/M47V/K54R/L85Q, S15P/Q33L/E35D/M47L/L85Q, E7D/E35D/M47I/L97Q, Q33L/E35D/T41S/M43I, E35D/M47I/K54R/L85E, Q33K/E35D/D46V/L85Q, Y31S/E35D/M47L/T79L/E88G, H18L/V22A/E35D/M47L/N48T/L85Q, Q27H/E35D/M47L/L85Q/R94Q/E95K, Q33K/E35D/M47V/K89E/K93R, E35D/M47I/E77A/L85Q/R94W, A26E/E35D/M43I/M47L/L85Q/K86E/R94W, Q27H/Q33L/E35D/M47V/N55D/L85Q/K89N, H18Y/V20A/Q33L/E35D/M47V/V53F, V22A/E35D/V68E/A71D, Q33L/E35D/M47L/A71G/F92S, V22A/R29H/E35D/D46E/M47I, Q33L/E35D/M43I/L85Q/R94W, H18Y/E35D/V68M/L97Q, Q33L/E35D/M47L/V68M/L85Q/E88D, Q33L/E35D/M43V/M47I/A71G, E35D/M47L/A71G/L97Q, E35D/M47V/A71G/L85M/L97Q, H18Y/Y31H/E35D/M47V/A71G/L85Q, E35D/D46E/M47V/L97Q, E35D/D46V/M47I/A71G/F92V, E35D/M47V/T62A/A71G/V83A/Y87H/L97M, Q33L/E35D/N48K/L85Q/L97Q, E35D/L85Q/K93T/E95V/L97Q, E35D/M47V/N48K/V68M/K89N, Q33L/E35D/M47I/N48D/A71G, R29H/E35D/M43V/M47I/I49V, Q27H/E35D/M47L/L85Q/D90G, E35D/M47L/L85Q/D90G, E35D/M47I/T62S/L85Q, A26E/E35D/M47L/A71G, E35D/M47I/Y87Q/K89E, V22A/E35D/M47I/Y87N, H18Y/A26E/E35D/M47L/L85Q/D90G, E35D/M47L/A71G/L85Q, E35D/M47V/A71G/E88D, E35D/A71G, E35D/M47V/A71G, I30V/E35D/M47V/A71G/A91V, I30V/V31C/E35D/M47V/A71G/L85M, V22D/E35D/M47L/L85Q, H18Y/E35D/N48K, E35D/T41S/M47V/A71G/K89N, E35D/M47V/N48T/L85Q, E35D/D46E/M47V/A71D/D90G, E35D/D46E/M47V/A71D, E35D/T41S/M43I/A71G/D90G, E35D/T41S/M43I/M47V/A71G, E35D/T41S/M43I/M47L/A71G, H18Y/Y22A/E35D/M47V/T62S/A71G, H18Y/A26E/E35D/M47L/V68M/A71G/D90G, E35D/K37E/M47V/N48D/L85Q/D90N, Q27H/E35D/D46V/M47L/A71G, V22L/Q27H/E35D/M47I/A71G, E35D/D46V/M47L/V68M/L85Q/E88D, E35D/T41S/M43V/M47I/L70M/A71G, E35D/D46E/M47V/N63D/L85Q, E35D/M47V/T62A/A71D/K93E, E35D/D46E/M47V/V68M/D90G/K93E, E35D/M43I/M47V/K89N, E35D/M47L/A71G/L85M/F92Y, E35D/M42V/M47V/E52D/L85Q, V22D/E35D/M47L/L70M/L97Q, E35D/T41S/M47V/L97Q, E35D/Y53H/A71G/D90G/L97R, E35D/A71D/L72V/R73H/E81K, Q33L/E35D/M43I/Y53F/T62S/L85Q, E35D/M38T/D46E/M47V/N48S, Q33R/E35D/M47V/N48K/L85M/F92L, E35D/M38T/M43V/M47V/N48R/L85Q, T28Y/Q33H/E35D/D46V/M47I/A71G, E35D/N48K/L72V, E35D/T41S/N48T, D46V/M47I/A71G, M47I/A71G, E35D/M43I/M47L/L85M, E35D/M43I/D46E/A71G/L85M, H18Y/E35D/M47L/A71G/A91S, E35D/M47I/N48K/I61F, E35D/M47V/T62S/L85Q, M43I/M47L/A71G, E35D/M47V, E35D/M47L/A71G/L85M, V22A/E35D/M47L/A71G, E35D/M47L/A71G, E35D/D46E/M47I, Q27H/E35D/M47I, E35D/D46E/L85M, E35D/D46E/A91G, E35D/D46E, E35D/L97R, H18Y/E35D, Q27L/E35D/M47V/I61V/L85M, E35D/M47V/I61V/L85M, E35D/M47V/L85M/R94Q, E35D/M47V/N48K/L85M, H18Y/E35D/M47V/N48K, A26E/Q27R/E35D/M47L/N48Y/L85Q, E35D/D46E/M47L/V68M/L85Q/F92L, E35D/M47I/T62S/L85Q/E88D, E24D/Q27R/E35D/T41S/M47V/L85Q, S15T/H18Y/E35D/M47V/T62A/N64S/A71G/L85Q/D90N, E35D/M47L/V68M/A71G/L85Q/D90G, H18Y/E35D/M47I/V68M/A71G/R94L, deltaE10-A98, Q33R/M47V/T62N/A71G, H18Y/Y22A/E35D/T41S/M47V/T62N/A71G/A91G, E35D/M47L/L70M, E35D/M47L/V68M, E35D/D46V/M47L/V68M/E88D, E35D/D46V/M47L/V68M/D90G, E35D/D46V/M47L/V68M/K89N, E35D/D46V/M47L/V68M/L85Q, E35D/D46V/M47L/V68M, E35D/D46V/M47L/V70M, E35D/D46V/M47L/V70M/L85Q, E35D/M47V/N48K/V68M, E24D/E35D/M47L/V68M/E95V/L97Q, E35D/D46E/M47I/T62A/V68M/L85M/Y87C, E35D/D46E/M47I/V68M/L85M, E35D/D46E/M47L/V68M/A71G/Y87C/K93R, E35D/D46E/M47L/V68M/T79M/L85M, E35D/D46E/M47L/V68M/T79M/L85M/L97Q, E35D/D46E/M47V/V68M/L85Q, E35D/M43I/M47L/V68M, E35D/M47I/V68M/Y87N, E35D/M47L/V68M/E95V/L97Q, E35D/M47L/Y53F/V68M/A71G/K93R/E95V, E35D/M47V/N48K/V68M/A71G/L85M, E35D/M47V/N48K/V68M/L85M, E35D/M47V/V68M/L85M, E35D/M47V/V68M/L85M/Y87D, E35D/T41S/D46E/M47I/V68M/K93R/E95V, H18Y/E35D/D46E/M47I/V68M/R94L, H18Y/E35D/M38I/M47L/V68M/L85M, H18Y/E35D/M47I/V68M/Y87N, H18Y/E35D/M47L/V68M/A71G/L85M, H18Y/E35D/M47L/V68M/E95V/L97Q, H18Y/E35D/M47L/Y53F/V68M/A71G, H18Y/E35D/M47L/Y53F/V68M/A71G/K93R/E95V, H18Y/E35D/M47V/V68M/L85M, H18Y/E35D/V68M/A71G/R94Q/E95V, H18Y/E35D/V68M/L85M/R94Q, H18Y/E35D/V68M/T79M/L85M, H18Y/Y22D/E35D/M47V/N48K/V68M, Q27L/Q33L/E35D/T41S/M47V/N48K/V68M/L85M, Q33L/E35D/M47V/T62S/V68M/L85M, Q33R/E35D/M38I/M47L/V68M, R29C/E35D/M47L/V68M/A71G/L85M, S21P/E35D/K37E/D46E/M47I/V68M, S21P/E35D/K37E/D46E/M47I/V68M/R94L, T13R/E35D/M47L/V68M, T13R/H18Y/E35D/V68M/L85M/R94Q, T13R/Q27L/Q33L/E35D/T41S/M47V/N48K/V68M/L85M, T13R/Q33L/E35D/M47L/V68M/L85M, T13R/Q33L/E35D/M47V/T62S/V68M/L85M, T13R/Q33R/E35D/M38I/M47L/V68M, T13R/Q33R/E35D/M38I/M47L/V68M/E95V/L97Q, T13R/Q33R/E35D/M38I/M47L/V68M/L85M, T13R/Q33R/E35D/M38I/M47L/V68M/L85M/R94Q, T13R/Q33R/E35D/M47L/V68M, T13R/Q33R/E35D/M47L/V68M/L85M, V22D/E24D/E35D/M47L/V68M, V22D/E24D/E35D/M47L/V68M/L85M/D90G, V22D/E24D/E35D/M47V/V68M, D46V, M47L, V68M, L85Q, E35D/D46V, E35D/V68M, E35D/L85Q, D46V/M47L, D46V/V68M, D46V/L85Q, M47L/V68M, M47L/L85Q, V68M/L85Q, E35D/D46V/M47L, E35D/D46V/V68M, E35D/D46V/L85Q, E35D/V68M/L85Q, D46V/M47L/V68M, D46V/M47L/L85Q, D46V/V68M/L85Q, M47L/V68M/L85Q, E35D/D46V/M47L/L85Q, E35D/D46V/V68M/L85Q, E35D/M47L/V68M/L85Q, D46V/M47L/V68M/L85Q, M47V, N48K, K89N, E35D/N48K, E35D/K89N, M47V/N48K, M47V/V68M, M47V/K89N, N48K/V68M, N48K/K89N, V68M/K89N, E35D/M47V/N48K, E35D/M47V/V68M, E35D/M47V/K89N, E35D/N48K/V68M, E35D/N48K/K89N, E35D/V68M/K89N, M47V/N48K/V68M, M47V/N48K/K89N, M47V/V68M/K89N, N48K/V68M/K89N, E35D/M47V/N48K/K89N, E35D/M47V/V68M/K89N, E35D/N48K/V68M/K89N, M47V/N48K/V68M/K89N, E35D/D46V/M47V/N48K/V68M, E35D/D46V/M47V/V68M/L85Q, E35D/D46V/M47V/V68M/K89N, E35D/M47V/N48K/V68M/L85Q, E35D/M47V/V68M/L85Q/K89N, A26E/E35D/M47L/V68M/A71G/D90G, H18Y/E35D/M47L/V68M/A71G/D90G, H18Y/A26E/M47L/V68M/A71G/D90G, H18Y/A26E/E35D/V68M/A71G/D90G, H18Y/A26E/E35D/M47L/A71G/D90G, H18Y/A26E/E35D/M47L/V68M/D90G, H18Y/A26E/E35D/M47L/V68M/A71G, E35D/M47L/V68M/A71G/D90G, H18Y/M47L/V68M/A71G/D90G, H18Y/A26E/V68M/A71G/D90G, H18Y/A26E/E35D/A71G/D90G, H18Y/A26E/E35D/M47L/D90G, H18Y/A26E/E35D/M47L/V68M, A26E/M47L/V68M/A71G/D90G, A26E/E35D/V68M/A71G/D90G, A26E/E35D/M47L/A71G/D90G, A26E/E35D/M47L/V68M/D90G, A26E/E35D/M47L/V68M/A71G, H18Y/E35D/V68M/A71G/D90G, H18Y/E35D/M47L/A71G/D90G, H18Y/E35D/M47L/V68M/D90G, H18Y/E35D/M47L/V68M/A71G, H18Y/A26E/M47L/A71G/D90G, H18Y/A26E/M47L/V68M/D90G, H18Y/A26E/M47L/V68M/A71G, H18Y/A26E/E35D/V68M/D90G, H18Y/A26E/E35D/V68M/A71G, H18Y/A26E/E35D/M47L/A71G, M47L/V68M/A71G/D90G, H18Y/V68M/A71G/D90G, H18Y/A26E/A71G/D90G, H18Y/A26E/E35D/D90G, H18Y/A26E/E35D/M47L, E35D/V68M/A71G/D90G, E35D/M47L/A71G/D90G, E35D/M47L/V68M/D90G, E35D/M47L/V68M/A71G, A26E/V68M/A71G/D90G, A26E/M47L/A71G/D90G, A26E/M47L/V68M/D90G, A26E/M47L/V68M/A71G, A26E/E35D/A71G/D90G, A26E/E35D/V68M/D90G, A26E/E35D/V68M/A71G, A26E/E35D/M47L/D90G, A26E/E35D/M47L/V68M, H18Y/M47L/A71G/D90G, H18Y/M47L/V68M/D90G, H18Y/M47L/V68M/A71G, H18Y/E35D/A71G/D90G, H18Y/E35D/V68M/D90G, H18Y/E35D/V68M/A71G, H18Y/E35D/M47L/D90G, H18Y/E35D/M47L/A71G, H18Y/E35D/M47L/V68M, H18Y/A26E/V68M/D90G, H18Y/A26E/V68M/A71G, H18Y/A26E/M47L/D90G, H18Y/A26E/M47L/A71G, H18Y/A26E/M47L/V68M, H18Y/A26E/E35D/A71G, H18Y/A26E/E35D/V68M, H18Y/E35D/M47V/V68M/A71G, H18C/A26P/E35D/M47L/V68M/A71G, H18I/A26P/E35D/M47V/V68M/A71G, H18L/A26N/D46E/V68M/A71G/D90G, H18L/E35D/M47V/V68M/A71G/D90G, H18T/A26N/E35D/M47L/V68M/A71G, H18V/A26K/E35D/M47L/V68M/A71G, H18V/A26N/E35D/M47V/V68M/A71G, H18V/A26P/E35D/M47V/V68L/A71G, H18V/A26P/E35D/M47L/V68M/A71G, H18V/E35D/M47V/V68M/A71G/D90G, H18Y/A26P/E35D/M47I/V68M/A71G, H18Y/A26P/E35D/M47V/V68M/A71G, H18Y/E35D/M47V/V68L/A71G/D90G, H18Y/E35D/M47V/V68M/A71G/D90G, A26P/E35D/M47I/V68M/A71G/D90G, H18V/A26G/E35D/M47V/V68M/A71G/D90G, H18V/A26S/E35D/M47L/V68M/A71G/D90G, H18V/A26R/E35D/M47L/V68M/A71G/D90G, H18V/A26D/E35D/M47V/V68M/A71G/D90G, H18V/A26Q/E35D/M47V/V68L/A71G/D90G, H18A/A26P/E35D/M47L/V68M/A71G/D90G, H18A/A26N/E35D/M47L/V68M/A71G/D90G, H18F/A26P/E35D/M47I/V68M/A71G/D90G, H18F/A26H/E35D/M47L/V68M/A71G/D90G, H18F/A26N/E35D/M47V/V68M/A71G/D90K, H18Y/A26N/E35D/M47F/V68M/A71G/D90G, H18Y/A26P/E35D/M47V/V68U/A71G/D90G, H18Y/A26Q/E35D/M47T/V68M/A71G/D90G, H18R/A26P/E35D/D46N/M47V/V68M/A71G/D90P, H18F/A26D/E35D/D46E/M47T/V68M/A71G/D90G.

In some embodiments, the variant CD80 polypeptide has one or more amino acid modifications (e.g., substitutions) in an unmodified CD80 or specific binding fragment there of corresponding to position(s) 7, 13, 15, 16, 20, 22, 23, 24, 25, 26, 27, 30, 31, 33, 34, 35, 36, 38, 41, 42, 43, 46, 47, 48, 51, 53, 54, 55, 57, 58, 61, 62, 65, 67, 68, 69, 70, 71, 72, 73, 74, 76, 77, 78, 79, 81, 82, 84, 85, 86, 87, 88, 92, 94, 95, and/or 97 with reference to numbering of SEQ ID NO: 2. In some embodiments, the variant CD80 polypeptide has one or more amino acid modifications (e.g., substitutions) in an unmodified CD80 or specific binding fragment there of corresponding to position(s) 7, 23, 26, 30, 34, 35, 46, 51, 55, 57, 58, 65, 71, 73, 78, 79, 82, or 84 with reference to numbering of SEQ ID NO: 2. In some embodiments, the variant CD80 polypeptide has a modification, e.g., amino acid substitution, at any 2 or more of the foregoing positions, such as 2, 3, 4, 5, 6, 7, 8, 9, 10, 11, 12, 13, 14, 15 or more of the positions.

In some embodiments, the variant CD80 polypeptide has one or more amino acid substitution selected from among E7D, T13A, T13R, S15P, S15T, C16R, H18A, H18C, H18F, H18I, H18T, H18V, V20A, V20I, V22D, V22I, V22L, E23D, E23G, E24D, L25S, A26D, A26E, A26G, A26H, A26K, A26N, A26P, A26Q, A26R, A26S, A26T, Q27H, Q27L, T28Y, I30F, I30T, I30V, Y31C, Y31S, Q33E, Q33K, Q33L, Q33R, K34E, E35D, E35G, K36R, T41S, M42I, M42V, M43L, M43T, D46E, D46N, D46V, M47F, M47I, M47L, M47V, M47Y, N48D, N48H, N48K, N48R, N48S, N48T, N48Y, P51A, Y53F, Y53H, K54E, K54N, K54R, N55D, N55I, T57A, T57I, I58V, I61F, I61V, T62A, T62N, N63D, L65P, I67L, I67V, V68E, V68I, V68L, I69F, L70M, L70P, L70Q, A71D, A71G, L72V, R73H, R73S, P74S, D76H, E77A, G78A, T79A, T79I, T79L, T79M, T79P, E81G, E81K, C82R, V84A, V84I, L85E, L85M, L85Q, K86M, Y87C, Y87D, Y87H, Y87Q, E88V, D90P, F92S, F92V, K93T, R94Q, R94W, E95D, E95V, L97M, and L97Q. In some embodiments, the variant CD80 polypeptide has one or more amino acid substitutions selected from E7D, E23D, E23G, A26E, A26P, A26S, A26T, I30F, I30T, I30V, K34E, E35D, E35G, D46E, D46V, P51A, N55D, N55I, T57A, T57I, I58V, L65P, A71D, A71G, R73S, G78A, T79A, T79I, T79L, T79P, C82R, V84A, V84I, L85Q, or a conservative amino acid substitution thereof. In some embodiments, the variant CD80 polypeptide comprises any one or more of the foregoing amino acid substitutions, such as 2, 3, 4, 5, 6, 7, 8, 9, 10, 11, 12, 13, 14, 15 or more of the amino acid substitutions. In some embodiments, the variant CD80 polypeptides comprises only one amino acid difference compared to the unmodified or wild-type CD80 polypeptide comprising only one of the foregoing amino acid substitutions.

In some embodiments, the variant CD80 polypeptide contains one or more additional amino acid modifications (e.g., substitutions) in an unmodified CD80 or specific binding fragment thereof corresponding to position(s) 12, 18, 29, 31, 37, 38, 41, 43, 44, 47, 61, 67, 68, 69, 70, 72, 77, 83, 88, 89, 90, 91, or 93 with reference to numbering of SEQ ID NO: 2. In some embodiments, the variant CD80 polypeptide has one or more additional amino acid substitution selected from among A12T, A12V, H18L, H18Y, R29H, Y31H, K37E, M38T, T41A, M43I, S44P, M47L, M47T, I67T, V68A, V68M, I69T, L70P, L70R, L70Q, L72P, E77G, V83A, V83I, E88D, K89E, K89N, D90G, D90N, A91T, K93R.

A conservative amino acid substitution is any amino acid that falls in the same class of amino acids as the substituted amino acids, other than the wild-type or unmodified amino acid. The classes of amino acids are aliphatic (glycine, alanine, valine, leucine, and isoleucine), hydroxyl or sulfur-containing (serine, cysteine, threonine, and methionine), cyclic (proline), aromatic (phenylalanine, tyrosine, tryptophan), basic (histidine, lysine, and arginine), and acidic/amide (aspartate, glutamate, asparagine, and glutamine). Thus, for example, a conservative amino acid substitution of the A26E substitution includes A26D, A26N, and A26Q amino acid substitutions.

In some embodiments, the variant CD80 polypeptide has one or more amino acid substitution selected from among L70Q, K89R, D90G, D90K, A91G, F92Y, K93R, I118V, T120S or T130A, with reference to numbering set forth in SEQ ID NO:2, or a conservative amino acid substitution thereof. In some embodiments, the variant CD80 polypeptide as two or more amino acids substitutions from among L70Q, K89R, D90G, D90K, A91G, F92Y, K93R, I118V, T120S or T130A, with reference to numbering set forth in SEQ ID NO:2, or a conservative amino acid substitution thereof. In some embodiments, the variant CD80 polypeptide as three or more amino acids substitutions from among L70Q, K89R, D90G, D90K, A91G, F92Y, K93R, I118V, T120S or T130A, with reference to numbering set forth in SEQ ID NO:2, or a conservative amino acid substitution thereof.

In some embodiments, the variant CD80 polypeptide has or comprises the amino acid substitutions L70Q/K89R, L70Q/D90G, L70Q/D90K, L70Q/A91G, L70Q/F92Y, L70Q/K93R, L70Q/I118V, L70Q/T120S, L70Q/T130A, K89R/D90G, K89R/D90K, K89R/A91G, K89R/F92Y, K89R/K93R, K89R/I118V, K89R/T120S, K89R/T130A, D90G/A91G, D90G/F92Y, D90G/K93R, D90G/I118V, D90G/T120S, D90G/T130A, D90K/A91G, D90K/F92Y, D90K/K93R, D90K/I118V, D90K/T120S, D90K/T130A, F92Y/K93R, F92Y/I118V, F92Y/T120S, F92Y/T130A, K93R/I118V, K93R/T120S, K93R/T130A, I118V/T120S, I118V/T130A or T120S/T130A.

In some embodiments, the variant CD80 polypeptide has or comprises the amino acid substitutions A91G/I118V/T120S/T130A.

In some embodiments, the variant CD80 polypeptide has or comprises the amino acid substitutions S21P/L70Q/D90G/I118V/T120S/T7130A.

In some embodiments, the variant CD80 polypeptide has or comprises the amino acid substitutions E88D/K89R/D90K/A91G/F92Y/K93R.

In some embodiments, the variant CD80 polypeptide has or comprises the amino acid substitutions I67T/L70Q/A91G/I118V/T120S/T130A.

In some embodiments, the variant CD80 polypeptide comprises an amino acid modification in an unmodified CD80 or specific binding fragment thereof at a position corresponding to position 18, with reference to numbering of positions set forth in SEQ ID NO:2. In some embodiments, the amino acid modification is the amino acid substitution H18Y or a conservative amino acid substitution thereof. In some embodiments, the variant CD80 polypeptide further contains one or more amino acid modifications, e.g. amino acid substitutions, at one or more positions 26, 35, 46, 47, 68, 71, 85 or 90. In some embodiments, the one or more amino acid modification is one or more amino acid substitutions A26E, E35D, D46E, D46V, M47I, M47L, V68M, A71G, L85Q or D90G, or a conservative amino acid substitution thereof. In some embodiments, the variant CD80 polypeptide comprises the amino acid modifications H18Y/A26E, H18Y/E35D, H18Y/D46E, H18Y/D46V, H18Y/M47I, H18Y/M47L, H18Y/V68M, H18Y/A71G, H18Y/L85Q, H18Y/D90G. The variant CD80 polypeptide can provide further amino acid modifications in accord with the provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 26, with reference to numbering of positions set forth in SEQ ID NO:2. In some embodiments, the amino acid modification is the amino acid substitution A26E or a conservative amino acid substitution thereof. In some embodiments, the variant CD80 polypeptide further contains one or more amino acid modifications, e.g. amino acid substitutions, at one or more positions 18, 35, 46, 47, 68, 71, 85 or 90. In some embodiments, the one or more amino acid modification is one or more amino acid substitutions H18Y, E35D, D46E, D46V, M47I, M47L, V68M, A71G, L85Q or D90G, or a conservative amino acid substitution thereof. In some embodiments, the variant CD80 polypeptide comprises the amino acid modifications H18Y/A26E, A26E/E35D, A26E/D46E, A26E/D46V, A26E/M47I, A26E/M47L, A26E/V68M, A26E/A71G, A26E/L85Q, A26E/D90G. The variant CD80 polypeptide can include further amino acid modifications, such as any described herein, in accord with provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 35, with reference to numbering of positions set forth in SEQ ID NO:2. In some embodiments, the amino acid modification is the amino acid substitution E35D or a conservative amino acid substitution thereof. In some embodiments, the variant CD80 polypeptide further contains one or more amino acid modifications, e.g. amino acid substitutions, at one or more positions 18, 26, 46, 47, 68, 71, 85 or 90. In some embodiments, the one or more amino acid modification is one or more amino acid substitutions H18Y, A26E, D46E, D46V, M47I, M47L, V68M, A71G, L85Q or D90G, or a conservative amino acid substitution thereof. In some embodiments, the variant CD80 polypeptide comprises the amino acid modifications H18Y/E35D, A26E/E35D, E35D/D46E, E35D/D46V, E35D/M47I, E35D/M47L, E35D/V68M, E35D/A71G, E35D/L85Q, E35D/D90G. The variant CD80 polypeptide can include further amino acid modifications, such as any described herein, in accord with provided embodiments. Table 2 sets forth exemplary amino acid modifications and variant CD80 polypeptides as described. In some embodiments, the variant CD80 polypeptide comprises an amino acid modification in an unmodified CD80 or specific binding fragment thereof at a position corresponding to position 46, with reference to numbering of positions set forth in SEQ ID NO:2. In some embodiments, the amino acid modification is the amino acid substitution D46E or D46V or a conservative amino acid substitution thereof. In some embodiments, the variant CD80 polypeptide further contains one or more amino acid modifications, e.g. amino acid substitutions, at one or more positions 18, 26, 35, 47, 68, 71, 85 or 90. In some embodiments, the one or more amino acid modification is one or more amino acid substitutions H18Y, A26E, E35D, M47I, M47L, V68M, A71G, L85Q or D90G, or a conservative amino acid substitution thereof. In some embodiments, the variant CD80 polypeptide comprises the amino acid modifications H18Y/D46E, A26E/D46E, E35D/D46E, D46E/M47I, D46E/M47L, D46E/V68M, D46E/A71G, D46E/L85Q, D46E/D90G. In some embodiments, the variant CD80 polypeptide comprises the amino acid modifications H18Y/D46V, A26E/D46V, E35D/D46V, D46V/M47I, D46V/M47L, D46V/V68M, D46V/A71G, D46V/L85Q, D46V/D90G. The variant CD80 polypeptide can include further amino acid modifications, such as any described herein, in accord with provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 47, with reference to numbering of positions set forth in SEQ ID NO:2. In some embodiments, the amino acid modification is the amino acid substitution M47I or M47L or a conservative amino acid substitution thereof. In some embodiments, the variant CD80 polypeptide further contains one or more amino acid modifications, e.g. amino acid substitutions, at one or more positions 18, 26, 35, 46, 68, 71, 85 or 90. In some embodiments, the one or more amino acid modification is one or more amino acid substitutions H18Y, A26E, E35D, D46E, D46V, V68M, A71G, L85Q or D90G, or a conservative amino acid substitution thereof. In some embodiments, the variant CD80 polypeptide comprises the amino acid modifications H18Y/M47I, A26E/M47I, E35D/M47I, M47I/D46E, M47I/D46V, M47I/V68M, M47I/A71G, M47I/L85Q or M47I/D90G. In some embodiments, the variant CD80 polypeptide comprises the amino acid modifications H18Y/M47L, A26E/M47L, E35D/M47L, M47L/D46E, M47L/D46V, M47L/V68M, M47L/A71G, M47L/L85Q, or M47L/D90G. The variant CD80 polypeptide can include further amino acid modifications, such as any described herein, in accord with provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 68, with reference to numbering of positions set forth in SEQ ID NO:2. In some embodiments, the amino acid modification is the amino acid substitution V68M or a conservative amino acid substitution thereof. In some embodiments, the variant CD80 polypeptide further contains one or more amino acid modifications, e.g. amino acid substitutions, at one or more positions 18, 26, 35, 46, 47, 71, 85 or 90. In some embodiments, the one or more amino acid modification is one or more amino acid substitutions H18Y, A26E, E35D, D46E, D46V, M47I, M47L, A71G, L85Q or D90G, or a conservative amino acid substitution thereof. In some embodiments, the variant CD80 polypeptide comprises the amino acid modifications H18Y/V68M, A26E/V68M, E35D/V68M, D46E/V68M, D46V/D68M, M47I/V68M, M47L/V68M, V68M/A71G, V68M/L85Q, V68M/D90G. The variant CD80 polypeptide can include further amino acid modifications, such as any described herein, in accord with provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 71, with reference to numbering of positions set forth in SEQ ID NO:2. In some embodiments, the amino acid modification is the amino acid substitution A71G or a conservative amino acid substitution thereof. In some embodiments, the variant CD80 polypeptide further contains one or more amino acid modifications, e.g. amino acid substitutions, at one or more positions 18, 26, 35, 46, 47, 68, 85 or 90. In some embodiments, the one or more amino acid modification is one or more amino acid substitutions H18Y, A26E, E35D, D46E, D46V, M47I, M47L, V68M, L85Q or D90G, or a conservative amino acid substitution thereof. In some embodiments, the variant CD80 polypeptide comprises the amino acid modifications H18Y/A71G, A26E/A71G, E35D/A71G, D46E/A71G, D46V/D68M, M47I/A71G, M47L/A71G, V68M/A71G, A71G/L85Q, A71G/D90G. The variant CD80 polypeptide can include further amino acid modifications, such as any described herein, in accord with provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 85, with reference to numbering of positions set forth in SEQ ID NO:2. In some embodiments, the amino acid modification is the amino acid substitution L85Q or a conservative amino acid substitution thereof. In some embodiments, the variant CD80 polypeptide further contains one or more amino acid modifications, e.g. amino acid substitutions, at one or more positions 18, 26, 35, 46, 47, 68, 71, or 90. In some embodiments, the one or more amino acid modification is one or more amino acid substitutions H18Y, A26E, E35D, D46E, D46V, M47I, M47L, V68M, A71G or D90G, or a conservative amino acid substitution thereof. In some embodiments, the variant CD80 polypeptide comprises the amino acid modifications H18Y/L85Q, A26E/L85Q, E35D/L85Q, D46E/L85Q, D46V/D68M, M47I/L85Q, M47L/L85Q, V68M/L85Q, A71G/L85Q, L85Q/D90G. The variant CD80 polypeptide can include further amino acid modifications, such as any described herein, in accord with provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 90, with reference to numbering of positions set forth in SEQ ID NO:2. In some embodiments, the amino acid modification is the amino acid substitution D90G or a conservative amino acid substitution thereof. In some embodiments, the variant CD80 polypeptide further contains one or more amino acid modifications, e.g. amino acid substitutions, at one or more positions 18, 26, 35, 46, 47, 68, 71, or 85. In some embodiments, the one or more amino acid modification is one or more amino acid substitutions H18Y, A26E, E35D, D46E, D46V, M47I, M47L, V68M, A71G or L85Q, or a conservative amino acid substitution thereof. In some embodiments, the variant CD80 polypeptide comprises the amino acid modifications H18Y/D90G, A26E/D90G, E35D/D90G, D46E/D90G, D46V/D68M, M47I/D90G, M47L/D90G, V68M/D90G, A71G/D90G, L85Q/D90G. The variant CD80 polypeptide can include further amino acid modifications, such as any described herein, in accord with provided embodiments. Table 2 sets forth exemplary amino acid modifications and variant CD80 polypeptides as described.

In some embodiments, the variant CD80 polypeptide comprises an amino acid modification in an unmodified CD80 or specific binding fragment thereof at a position corresponding to position 18, 26, 35, 46, 47, 48, 68, 70, 71, 85, 88, 89, 90, or 93 with reference to numbering of positions set forth in SEQ ID NO:2. In some embodiments, the amino acid modification is the amino acid substitution H18Y, A26E, E35D, D46E, D46V, M47I, M47L, M47V, N48K, V68M, L70M, A71G, L85Q, E88D, K89N, D90G, K93E or a conservative amino acid substitution thereof. In some embodiments, the variant CD80 polypeptide comprises the amino acid modifications E35D/M47I/L70M, E35D/M47L E35D/M47V/N48K/V68M/K89N, H18Y/A26E/E35D/M47L/V68M/A71G/D90G, E35D/D46E/M47V/V68M/D90G/K93E, or E35D/D46V/M47L/V68M/L85Q/E88D.

In some embodiments, the variant CD80 polypeptide does not contain amino acid modifications in an unmodified CD80 polypeptide set forth in SEQ ID NO:2, 76 or 150 in which the only amino acid modifications are H18Y/M47I/T57I/A71G, H18Y/A26T/E35D/A71D/L85Q or H18Y/A71D/L72P/E88V. In some embodiments, the variant CD80 polypeptide is not the polypeptide set forth in SEQ ID NO: 41, 59, 66, 115, 133, 140, 189, 207 or 214.

In some embodiments, the variant CD80 polypeptide does not contain amino acid modifications in an unmodified CD80 polypeptide set forth in SEQ ID NO:2, 76 or 150 in which the only amino acid modifications are A26E/E35D/M47L/L85Q. In some embodiments, the variant CD80 polypeptide is not the polypeptide set forth in SEQ ID NO: 73, 147, or 221.

In some embodiments, the variant CD80 polypeptide does not contain amino acid modifications in an unmodified CD80 polypeptide set forth in SEQ ID NO:2, 76 or 150 in which the only amino acid modifications are E35D/M47I/L65P/D90N, L25S/E35D/M47I/D90N, E35D/A71D, E35D/M47I, E35D/T57I/L70Q/A71D, E35D/A71D, E35D/I67L/A71D. E35D, E35D/M47I/L70M, E35D/A71D/L72V, E35D/M43L/L70M, A26P/E35D/M43I/L85Q/E88D, E35D/D46V/L85Q, Q27L/E35D/M47/T57I/L70Q/E88D, E35D/T57A/A71D/L85Q, H18Y/A26T/E35D/A71D/L85Q, E35D/M47L, E35D/M43I/A71D, E23G/A26S/E35D/T62N/A71D/L72V/L85M, A12T/E24D/E35D/D46V/I61V/L72P/E95V, V22L/E35D/M43L/A71G/D76H, A26E/E35D/M47L/L85Q, Y31H/E35D/T41S/V68L/K93R/R94W. In some embodiments, the variant CD80 polypeptide is not the polypeptide set forth in SEQ ID NO: 19, 20, 28, 29, 37, 46, 47, 50, 51, 52, 53, 54, 55, 56, 58, 59, 60, 64, 68, 69, 70, 73, 75, 93, 94, 102, 103, 111, 120, 121, 124, 125, 126, 127, 128, 129, 130, 132, 133, 134, 138, 142, 143, 144, 147, 149, 167, 168, 176, 177, 185, 194, 195, 198, 199, 200, 201, 202, 203, 204, 206, 207, 208, 212, 216, 217, 218, 221, or 223.

In some embodiments, the variant CD80 polypeptide does not contain amino acid modifications in an unmodified CD80 polypeptide set forth in SEQ ID NO:2, 76 or 150 in which the only amino acid modifications are E35D/D46V/L85Q, A12T/E24D/E35D/D46V/I61V/L72P/E95V or D46E/A71D. In some embodiments, the variant CD80 polypeptide is not the polypeptide set forth in SEQ ID NO: 55, 69, 74, 129, 143, 148, 203, 217, or 222.

In some embodiments, the variant CD80 polypeptide does not contain amino acid modifications in an unmodified CD80 polypeptide set forth in SEQ ID NO:2, 76 or 150 in which the only amino acid modifications are E35D/M47I/L65P/D90N, L25S/E35D/M47I/D90N, E35D/M47I, M47L/V68A, M47I/E88D, H18Y/M47/T57I/A71G, T13R/M42V/M47I/A71D, E35D/M47I/L70M, Q27L/E35D/M47I/T57I/L70Q/E88D, E35D/M47L, A26E/E35D/M47L/L85Q. In some embodiments, the variant CD80 polypeptide is not the polypeptide set forth in SEQ ID NO: 19, 20, 29, 33, 38, 41, 49, 51, 56, 60, 73, 93, 94, 103, 107, 112, 115, 123, 125, 130, 134, 147, 167, 168, 177, 181, 186, 189, 197, 199, 204, 208, 221.

In some embodiments, the variant CD80 polypeptide does not contain amino acid modifications in an unmodified CD80 polypeptide set forth in SEQ ID NO:2, 76 or 150 in which the only amino acid modifications are A26E/E35D/M47L/L85Q. In some embodiments, the variant CD80 polypeptide is not the polypeptide set forth in SEQ ID NO: 62, 136, 210.

In some embodiments, the variant CD80 polypeptide does not contain amino acid modifications in an unmodified CD80 polypeptide set forth in SEQ ID NO:2, 76 or 150 in which the only amino acid modifications are H18Y/M47I/T57I/A71G or V22L/E35D/M43L/A71G/D76H. In some embodiments, the variant CD80 polypeptide is not the polypeptide set forth in SEQ ID NO: 41, 70, 115, 144, 189 or 218.

In some embodiments, the variant CD80 polypeptide does not contain amino acid modifications in an unmodified CD80 polypeptide set forth in SEQ ID NO:2, 76 or 150 in which the only amino acid modifications are A26P/E35D/M43I/L85Q/E88D, E35D/D46V/L85Q, E35D/T57A/A71D/L85Q, H18Y/A26T/E35D/A71D/L85Q or A26E/E35D/M47L/L85Q. In some embodiments, the variant CD80 polypeptide is not the polypeptide set forth in SEQ ID NO: 54, 55, 58, 59, 73, 128, 129, 132, 133, 147, 202, 203, 206, 207 or 221.

In some embodiments, the variant CD80 polypeptide comprises amino acid modifications in an unmodified CD80 or specific binding fragment thereof at a position corresponding to E35D and M47L. In some embodiments, the variant CD80 polypeptide comprises amino acid modifications in an unmodified CD80 or specific binding fragment thereof corresponding to E35D and M47I. In some embodiments, the variant CD80 polypeptide comprises amino acid modifications in an unmodified CD80 or specific binding fragment thereof corresponding to E35D and A71G. In some embodiments, the variant CD80 polypeptide comprises amino acid modifications in an unmodified CD80 or specific binding fragment thereof corresponding to E35D and M47V. In some embodiments, the variant CD80 polypeptide comprises amino acid modifications in an unmodified CD80 or specific binding fragment thereof corresponding to E35D and V68M. In some embodiments, the variant CD80 polypeptide comprises amino acid modifications in an unmodified CD80 or specific binding fragment thereof corresponding to H18Y and E35D.

In some embodiments, the variant CD80 polypeptide comprises at least three amino acid modifications, wherein the at least three modifications include a modification at three or more of positions corresponding to positions 18, 26, 35, 46, 47, 68, 71, 85 or 90, with reference to numbering of positions set forth in SEQ ID NO:2. In some embodiments, the at least three amino acid modification comprises amino acid modifications in an unmodified CD80 or specific binding fragment thereof corresponding to H18Y, A26E, E35D, D46E, D46V, M47I, M47L, V68M, A71G, L85Q, or D90G or a conservative amino acid substitution thereof.

In some embodiments, the variant CD80 polypeptide comprises amino acid modifications in an unmodified CD80 or specific binding fragment thereof corresponding to E35D/M47L/V68M.

In some embodiments, the variant CD80 polypeptide comprises amino acid modifications in an unmodified CD80 or specific binding fragment thereof corresponding to E35D/M47V/V68M.

In some embodiments, the variant CD80 polypeptide comprises amino acid modifications in an unmodified CD80 or specific binding fragment thereof corresponding to E35D/M47L/L85Q.

In some embodiments, the variant CD80 polypeptide comprises amino acid modifications in an unmodified CD80 or specific binding fragment thereof corresponding to H18Y/E35D/M47I.

In some embodiments, the variant CD80 polypeptide comprises any of the substitutions (mutations) listed in Table 2. Table 2 also provides exemplary sequences by reference to SEQ ID NO for the extracellular domain (ECD) or IgV domain of wild-type CD80 or exemplary variant CD80 polypeptides. As indicated, the exact locus or residues corresponding to a given domain can vary, such as depending on the methods used to identify or classify the domain. Also, in some cases, adjacent N- and/or C-terminal amino acids of a given domain (e.g., IgV) also can be included in a sequence of a variant IgSF polypeptide, such as to ensure proper folding of the domain when expressed. Thus, it is understood that the exemplification of the SEQ ID NOs in Table 2 is not to be construed as limiting. For example, the particular domain, such as the IgV domain, of a variant CD80 polypeptide can be several amino acids longer or shorter, such as 1-10, e.g., 1, 2, 3, 4, 5, 6 or 7 amino acids longer or shorter, than the sequence of amino acids set forth in the respective SEQ ID NO.

In some embodiments, the variant CD80 polypeptide comprises any of the extracellular domain (ECD) sequences listed in Table 2 (i.e., any one of SEQ ID NOS: 3-75, 224-319, 512-722, 1145-1175, 1299-1365, 1383-1444, 1447-1500, 1537 or 1541). In some embodiments, the variant CD80 polypeptide comprises a polypeptide sequence that exhibits at least 90% identity, at least 91% identity, at least 92% identity, at least 93% identity, at least 94% identity, at least 95% identity, such as at least 96% identity, 97% identity, 98% identity, or 99% identity to any of the extracellular domain (ECD) sequences listed in Table 2 (i.e., any one of SEQ ID NOS: 3-75, 224-319, 512-722, 1145-1175, 1299-1365, 1383-1444, 1447-1500, 1537 or 1541) and contains the amino acid modification(s), e.g., substitution(s), not present in the wild-type or unmodified CD80. In some embodiments, the variant CD80 polypeptide comprises a specific binding fragment of any of the extracellular domain (ECD) sequences listed in Table 2 (i.e., any one of SEQ ID NOS: 3-75, 224-319, 512-722, 1145-1175, 1299-1365, 1383-1444, 1447-1500, 1537 or 1541) and contains the amino acid modification(s), e.g., substitution(s), not present in the wild-type or unmodified CD80. In some embodiments, the variant CD80 polypeptide comprises any of the IgV sequences listed in Table 2 (i.e., any one of SEQ ID NOS: 77-149, 151-223, 320-511, 723-1144, 1176-1237, 1256-1298, 1366-1368, 1370-1380, 1381-1382, 1445-1446, 1538, 1540, 1542 or 1544). In some embodiments, the variant CD80 polypeptide comprises a polypeptide sequence that exhibits at least 90% identity, at least 91% identity, at least 92% identity, at least 93% identity, at least 94% identity, at least 95% identity, such as at least 96% identity, 97% identity, 98% identity, or 99% identity to any of the IgV sequences listed in Table 2 (i.e., any one of SEQ ID NOS: 77-149, 151-223, 320-511, 723-1144, 1176-1237, 1256-1298,1366-1368, 1370-1380, 1381-1382, 1445-1446, 1538, 1540, 1542 or 1544) and contains the amino acid modification(s), e.g., substitution(s), not present in the wild-type or unmodified CD80. In some embodiments, the variant CD80 polypeptide comprises a specific binding fragment of any of the IgV sequences listed in Table 2 (i.e., any one of SEQ ID NOS: 77-149, 151-223, 320-511, 723-1144, 1176-1237, 1256-1298,1366-1368, 1370-1380, 1381-1382, 1445-1446, 1538, 1540, 1542 or 1544) and contains the amino acid modification(s), e.g., substitution(s), not present in the wild-type or unmodified CD80.

Table 2 also provides exemplary sequences by reference to SEQ ID NO for the extracellular domain (ECD) or IgV domain of wild-type CD80 or exemplary variant CD80 polypeptides. As indicated, the exact locus or residues corresponding to a given domain can vary, such as depending on the methods used to identify or classify the domain. Also, in some cases, adjacent N- and/or C-terminal amino acids of a given domain (e.g., ECD) also can be included in a sequence of a variant IgSF polypeptide, such as to ensure proper folding of the domain when expressed. Thus, it is understood that the exemplification of the SEQ ID NOS in Table 2 is not to be construed as limiting. For example, the particular domain, such as the IgV domain, of a variant CD80 polypeptide can be several amino acids longer or shorter, such as 1-10, e.g., 1, 2, 3, 4, 5, 6 or 7, amino acids longer or shorter, than the sequence of amino acids set forth in the respective SEQ ID NO.

TABLE 2
Exemplary variant CD80 polypeptides

| | ECD | IgV | |
|---|---|---|---|
| | SEQ | SEQ | |
| | ID | ID | |
| CD80 Mutation(s) | NO | NO | |
| Wild-type | 2 | 76 | 150 |
| L70P | 3 | 77 | 151 |
| I30F/L70P | 4 | 78 | 152 |
| Q27H/T41S/A71D | 5 | 79 | 153 |
| I30T/L70R | 6 | 80 | 154 |
| T13R/C16R/L70Q/A71D | 7 | 81 | 155 |
| T57I | 8 | 82 | 156 |
| M43I/C82R | 9 | 83 | 157 |
| V22L/M38V/M47T/A71D/L85M | 10 | 84 | 158 |
| I30V/T57I/L70P/A71D/A91T | 11 | 85 | 159 |
| V22I/L70M/A71D | 12 | 86 | 160 |
| N55D/L70P/E77G | 13 | 87 | 161 |
| T57A/I69T | 14 | 88 | 162 |
| N55D/K86M | 15 | 89 | 163 |
| L72P/T79I | 16 | 90 | 164 |
| L70P/F92S | 17 | 91 | 165 |
| T79P | 18 | 92 | 166 |
| E35D/M47I/L65P/D90N | 19 | 93 | 167 |
| L25S/E35D/M47I/D90N | 20 | 94 | 168 |
| A71D | 22 | 96 | 170 |
| T13A/I61N/A71D | 23 | 97 | 171 |
| E81K/A91S | 24 | 98 | 172 |
| A12V/M47V/L70M | 25 | 99 | 173 |
| K34E/T41A/L72V | 26 | 100 | 174 |
| T41S/A71D/V84A | 27 | 101 | 175 |
| E35D/A71D | 28 | 102 | 176 |
| E35D/M47I | 29 | 103 | 177 |
| K36R/G78A | 30 | 104 | 178 |
| Q33E/T41A | 31 | 105 | 179 |
| M47V/N48H | 32 | 106 | 180 |
| M47L/V68A | 33 | 107 | 181 |
| S44P/A71D | 34 | 108 | 182 |
| Q27H/M43I/A71D/R73S | 35 | 109 | 183 |
| E35D/T57I/L70Q/A71D | 37 | 111 | 185 |
| M47I/E88D | 38 | 112 | 186 |
| M42I/I61V/A71D | 39 | 113 | 187 |
| P51A/A71D | 40 | 114 | 188 |
| H18Y/M47I/T57I/A71G | 41 | 115 | 189 |
| V20I/M47V/T57I/V84I | 42 | 116 | 190 |
| V20I/M47V/A71D | 43 | 117 | 191 |
| A71D/L72V/E95K | 44 | 118 | 192 |
| V22L/E35G/A71D/L72P | 45 | 119 | 193 |
| E35D/A71D | 46 | 120 | 194 |
| E35D/I67L/A71D | 47 | 121 | 195 |
| Q27H/E35G/A71D/L72P/T79I | 48 | 122 | 196 |
| T13R/M42V/M47I/A71D | 49 | 123 | 197 |
| E35D | 50 | 124 | 198 |
| E35D/M47I/L70M | 51 | 125 | 199 |
| E35D/A71D/L72V | 52 | 126 | 200 |
| E35D/M43L/L70M | 53 | 127 | 201 |
| A26P/E35D/M43I/L85Q/E88D | 54 | 128 | 202 |
| E35D/D46V/L85Q | 55 | 129 | 203 |
| Q27L/E35D/M47I/T57I/L70Q/E88D | 56 | 130 | 204 |
| M47V/I69F/A71D/V83I | 57 | 131 | 205 |
| E35D/T57A/A71D/L85Q | 58 | 132 | 206 |
| H18Y/A26T/E35D/A71D/L85Q | 59 | 133 | 207 |
| E35D/M47L | 60 | 134 | 208 |
| E23D/M42V/M43I/I58V/L70R | 61 | 135 | 209 |
| V68M/L70M/A71D/E95K | 62 | 136 | 210 |
| N55I/T57I/I69F | 63 | 137 | 211 |
| E35D/M43I/A71D | 64 | 138 | 212 |
| T41S/T57I/L70R | 65 | 139 | 213 |
| H18Y/A71D/L72P/E88V | 66 | 140 | 214 |
| V20I/A71D | 67 | 141 | 215 |
| E23G/A26S/E35D/T62N/A71D/L72V/L85M | 68 | 142 | 216 |
| A12T/E24D/E35D/D46V/I61V/L72P/E95V | 69 | 143 | 217 |
| V22L/E35D/M43L/A71G/D76H | 70 | 144 | 218 |
| E35G/K54E/A71D/L72P | 71 | 145 | 219 |
| L70Q/A71D | 72 | 146 | 220 |
| A26E/E35D/M47L/L85Q | 73 | 147 | 221 |
| D46E/A71D | 74 | 148 | 222 |
| Y31H/E35D/T41S/V68L/K93R/R94W | 75 | 149 | 223 |
| A26E/Q33R/E35D/M47L/L85Q/K86E | 224 | 320 | 416 |
| A26E/Q33R/E35D/M47L/L85Q | 225 | 321 | 417 |
| E35D/M47L/L85Q | 226 | 322 | 418 |
| A26E/Q33L/E35D/M47L/L85Q | 227 | 323 | 419 |
| A26E/Q33L/E35D/M47L | 228 | 324 | 420 |
| H18Y/A26E/Q33L/E35D/M47L/L85Q | 229 | 325 | 421 |
| Q33L/E35D/M47I | 230 | 326 | 422 |
| H18Y/Q33L/E35D/M47I | 231 | 327 | 423 |
| Q33L/E35D/D46E/M47I | 232 | 328 | 424 |
| Q33R/E35D/D46E/M47I | 233 | 329 | 425 |
| H18Y/E35D/M47L | 234 | 330 | 426 |
| Q33L/E35D/M47V | 235 | 331 | 427 |
| Q33L/E35D/M47V/T79A | 236 | 332 | 428 |
| Q33L/E35D/T41S/M47V | 237 | 333 | 429 |
| Q33L/E35D/M47I/L85Q | 238 | 334 | 430 |
| Q33L/E35D/M47I/T62N/L85Q | 239 | 335 | 431 |
| Q33L/E35D/M47V/L85Q | 240 | 336 | 432 |
| A26E/E35D/M43T/M47L/L85Q/R94Q | 241 | 337 | 433 |
| Q33R/E35D/K37E/M47V/L85Q | 242 | 338 | 434 |
| V22A/E23D/Q33L/E35D/M47V | 243 | 339 | 435 |
| E24D/Q33L/E35D/M47V/K54R/L85Q | 244 | 340 | 436 |
| S15P/Q33L/E35D/M47L/L85Q | 245 | 341 | 437 |
| E7D/E35D/M47I/L97Q | 246 | 342 | 438 |
| Q33L/E35D/T41S/M43I | 247 | 343 | 439 |
| E35D/M47I/K54R/L85E | 248 | 344 | 440 |
| Q33K/E35D/D46V/L85Q | 249 | 345 | 441 |
| Y31S/E35D/M47L/T79L/E88G | 250 | 346 | 442 |
| H18L/V22A/E35D/M47L/N48T/L85Q | 251 | 347 | 443 |
| Q27H/E35D/M47L/L85Q/R94Q/E95K | 252 | 348 | 444 |
| Q33K/E35D/M47V/K89E/K93R | 253 | 349 | 445 |
| E35D/M47I/E77A/L85Q/R94W | 254 | 350 | 446 |
| A26E/E35D/M43I/M47L/L85Q/K86E/R94W | 255 | 351 | 447 |
| Q27H/Q33L/E35D/M47V/N55D/L85Q/K89N | 256 | 352 | 448 |
| H18Y/V20A/Q33L/E35D/M47V/Y53F | 257 | 353 | 449 |
| V22A/E35D/V68E/A71D | 258 | 354 | 450 |
| Q33L/E35D/M47L/A71G/F92S | 259 | 355 | 451 |
| V22A/R29H/E35D/D46E/M47I | 260 | 356 | 452 |
| Q33L/E35D/M43I/L85Q/R94W | 261 | 357 | 453 |
| H18Y/E35D/V68M/L97Q | 262 | 358 | 454 |
| Q33L/E35D/M47L/V68M/L85Q/E88D | 263 | 359 | 455 |
| Q33L/E35D/M43V/M47I/A71G | 264 | 360 | 456 |
| E35D/M47L/A71G/L97Q | 265 | 361 | 457 |
| E35D/M47V/A71G/L85M/L97Q | 266 | 362 | 458 |
| H18Y/Y31H/E35D/M47V/A71G/L85Q | 267 | 363 | 459 |
| E35D/D46E/M47V/L97Q | 268 | 364 | 460 |
| E35D/D46V/M47I/A71G/F92V | 269 | 365 | 461 |
| E35D/M47V/T62A/A71GN83A/Y87H/L97M | 270 | 366 | 462 |
| Q33L/E35D/N48K/L85Q/L97Q | 271 | 367 | 463 |
| E35D/L85Q/K93T/E95V/L97Q | 272 | 368 | 464 |
| E35D/M47V/N48K/V68M/K89N | 273 | 369 | 465 |
| Q33L/E35D/M47I/N48D/A71G | 274 | 370 | 466 |
| R29H/E35D/M43V/M47I/I49V | 275 | 371 | 467 |
| Q27H/E35D/M47I/L85Q/D90G | 276 | 372 | 468 |
| E35D/M47I/L85Q/D90G | 277 | 373 | 469 |
| E35D/M47I/T62S/L85Q | 278 | 374 | 470 |
| A26E/E35D/M47L/A71G | 279 | 375 | 471 |
| E35D/M47I/Y87Q/K89E | 280 | 376 | 472 |
| V22A/E35D/M47I/Y87N | 281 | 377 | 473 |
| H18Y/A26E/E35D/M47L/L85Q/D90G | 282 | 378 | 474 |
| E35D/M47L/A71G/L85Q | 283 | 379 | 475 |
| E35D/M47V/A71G/E88D | 284 | 380 | 476 |
| E35D/A71G | 285 | 381 | 477 |
| E35D/M47V/A71G | 286 | 382 | 478 |
| I30V/E35D/M47V/A71G/A91V | 287 | 383 | 479 |
| I30V/Y31C/E35D/M47V/A71G/L85M | 288 | 384 | 480 |
| V22D/E35D/M47L/L85Q | 289 | 385 | 481 |
| H18Y/E35D/N48K | 290 | 386 | 482 |
| E35D/T41S/M47V/A71G/K89N | 291 | 387 | 483 |
| E35D/M47V/N48T/L85Q | 292 | 388 | 484 |
| E35D/D46E/M47V/A71D/D90G | 293 | 389 | 485 |
| E35D/D46E/M47V/A71D | 294 | 390 | 486 |
| E35D/T41S/M43I/A71G/D90G | 295 | 391 | 487 |
| E35D/T41S/M43I/M47V/A71G | 296 | 392 | 488 |
| E35D/T41S/M43I/M47L/A71G | 297 | 393 | 489 |
| H18Y/V22A/E35D/M47V/T62S/A71G | 298 | 394 | 490 |
| H18Y/A26E/E35D/M47L/V68M/A71G/D90G | 299 | 395 | 491 |
| E35D/K37E/M47V/N48D/L85Q/D90N | 300 | 396 | 492 |
| Q27H/E35D/D46V/M47L/A71G | 301 | 397 | 493 |
| V22L/Q27H/E35D/M47I/A71G | 302 | 398 | 494 |
| E35D/D46V/M47L/V68M/L85Q/E88D | 303 | 399 | 495 |
| E35D/T41S/M43V/M47I/L70M/A71G | 304 | 400 | 496 |
| E35D/D46E/M47V/N63D/L85Q | 305 | 401 | 497 |
| E35D/M47V/T62A/A71D/K93E | 306 | 402 | 498 |
| E35D/D46E/M47V/V68M/D90G/K93E | 307 | 403 | 499 |
| E35D/M43I/M47V/K89N | 308 | 404 | 500 |
| E35D/M47L/A71G/L85M/F92Y | 309 | 405 | 501 |
| E35D/M42V/M47V/E52D/L85Q | 310 | 406 | 502 |
| V22D/E35D/M47L/L70M/L97Q | 311 | 407 | 503 |
| E35D/T41S/M47V/L97Q | 312 | 408 | 504 |
| E35D/Y53H/A71G/D90G/L97R | 313 | 409 | 505 |
| E35D/A71D/L72V/R73H/E81K | 314 | 410 | 506 |
| Q33L/E35D/M43I/Y53F/T62S/L85Q | 315 | 411 | 507 |
| E35D/M38T/D46E/M47V/N48S | 316 | 412 | 508 |
| Q33R/E35D/M47V/N48K/L85M/F92L | 317 | 413 | 509 |
| E35D/M38T/M43V/M47V/N48R/L85Q | 318 | 414 | 510 |
| T28Y/Q33H/E35D/D46V/M47I/A71G | 319 | 415 | 511 |
| E35D/N48K/L72V | 512 | 723 | 934 |
| E35D/T41S/N48T | 513 | 724 | 935 |
| D46V/M47I/A71G | 514 | 725 | 936 |
| M47I/A71G | 515 | 726 | 937 |
| E35D/M43I/M47L/L85M | 516 | 727 | 938 |
| E35D/M43I/D46E/A71G/L85M | 517 | 728 | 939 |
| H18Y/E35D/M47L/A71G/A91S | 518 | 729 | 940 |
| E35D/M47I/N48K/I61F | 519 | 730 | 941 |
| E35D/M47V/T62S/L85Q | 520 | 731 | 942 |
| M43I/M47L/A71G | 521 | 732 | 943 |
| E35D/M47V | 522 | 733 | 944 |
| E35D/M47L/A71G/L85M | 523 | 734 | 945 |
| V22A/E35D/M47L/A71G | 524 | 735 | 946 |
| E35D/M47L/A71G | 525 | 736 | 947 |
| E35D/D46E/M47I | 526 | 737 | 948 |
| Q27H/E35D/M47I | 527 | 738 | 949 |
| E35D/D46E/L85M | 528 | 739 | 950 |
| E35D/D46E/A91G | 529 | 740 | 951 |
| E35D/D46E | 530 | 741 | 952 |
| E35D/L97R | 531 | 742 | 953 |
| H18Y/E35D | 532 | 743 | 954 |
| Q27L/E35D/M47V/I61V/L85M | 533 | 744 | 955 |
| E35D/M47V/I61V/L85M | 534 | 745 | 956 |
| E35D/M47V/L85M/R94Q | 535 | 746 | 957 |
| E35D/M47V/N48K/L85M | 536 | 747 | 958 |
| H18Y/E35D/M47V/N48K | 537 | 748 | 959 |
| A26E/Q27R/E35D/M47L/N48Y/L85Q | 538 | 749 | 960 |
| E35D/D46E/M47L/V68M/L85Q/F92L | 539 | 750 | 961 |
| E35D/M47I/T62S/L85Q/E88D | 540 | 751 | 962 |
| E24D/Q27R/E35D/T41S/M47V/L85Q | 541 | 752 | 963 |
| S15T/H18Y/E35D/M47V/T62A/ | 542 | 753 | 964 |
| N64S/A71G/L85Q/D90N | | | |
| E35D/M47L/V68M/A71G/L85Q/D90G | 543 | 754 | 965 |
| H18Y/E35D/M47I/V68M/A71G/R94L | 544 | 755 | 966 |
| deltaE10-A98 | 545 | 756 | 967 |
| Q33R/M47V/T62N/A71G | 546 | 757 | 968 |
| H18Y/V22A/E35D/T41S/M47V/T62N/ | 547 | 758 | 969 |
| A71G/A91G | | | |
| E35D/M47L/L70M | 548 | 759 | 970 |
| E35D/M47L/V68M | 549 | 760 | 971 |
| E35D/D46V/M47L/V68M/E88D | 550 | 761 | 972 |
| E35D/D46V/M47L/V68M/D90G | 551 | 762 | 973 |
| E35D/D46V/M47L/V68M/K89N | 552 | 763 | 974 |
| E35D/D46V/M47L/V68M/L85Q | 553 | 764 | 975 |
| E35D/D46V/M47L/V68M | 554 | 765 | 976 |
| E35D/D46V/M47L/V70M | 555 | 766 | 977 |
| E35D/D46V/M47L/V70M/L85Q | 556 | 767 | 978 |
| E35D/M47V/N48K/V68M | 557 | 768 | 979 |
| E24D/E35D/M47L/V68M/E95V/L97Q | 558 | 769 | 980 |
| E35D/D46E/M47I/T62A/V68M/L85M/Y87C | 559 | 770 | 981 |
| E35D/D46E/M47I/V68M/L85M | 560 | 771 | 982 |
| E35D/D46E/M47L/V68M/A71G/Y87C/K93R | 561 | 772 | 983 |
| E35D/D46E/M47L/V68M/T79M/L85M | 562 | 773 | 984 |
| E35D/D46E/M47L/V68M/T79M/L85M/L97Q | 563 | 774 | 985 |
| E35D/D46E/M47V/V68M/L85Q | 564 | 775 | 986 |
| E35D/M43I/M47L/V68M | 565 | 776 | 987 |
| E35D/M47I/V68M/Y87N | 566 | 777 | 988 |
| E35D/M47L/V68M/E95V/L97Q | 567 | 778 | 989 |
| E35D/M47L/Y53F/V68M/A71G/K93R/E95V | 568 | 779 | 990 |
| E35D/M47V/N48K/V68M/A71G/L85M | 569 | 780 | 991 |
| E35D/M47V/N48K/V68M/L85M | 570 | 781 | 992 |
| E35D/M47V/V68M/L85M | 571 | 782 | 993 |
| E35D/M47V/V68M/L85M/Y87D | 572 | 783 | 994 |
| E35D/T41S/D46E/M47I/V68M/K93R/E95V | 573 | 784 | 995 |
| H18Y/E35D/D46E/M47I/V68M/R94L | 574 | 785 | 996 |
| H18Y/E35D/M38I/M47L/V68M/L85M | 575 | 786 | 997 |
| H18Y/E35D/M47I/V68M/Y87N | 576 | 787 | 998 |
| H18Y/E35D/M47L/V68M/A71G/L85M | 577 | 788 | 999 |
| H18Y/E35D/M47L/V68M/E95V/L97Q | 578 | 789 | 1000 |
| H18Y/E35D/M47L/Y53F/V68M/A71G | 579 | 790 | 1001 |
| H18Y/E35D/M47L/Y53F/V68M/ | 580 | 791 | 1002 |
| A71G/K93R/E95V | | | |
| H18Y/E35D/M47V/V68M/L85M | 581 | 792 | 1003 |
| H18Y/E35D/V68M/A71G/R94Q/E95V | 582 | 793 | 1004 |
| H18Y/E35D/V68M/L85M/R94Q | 583 | 794 | 1005 |
| H18Y/E35D/V68M/T79M/L85M | 584 | 795 | 1006 |
| H18Y/V22D/E35D/M47V/N48K/V68M | 585 | 796 | 1007 |
| Q27L/Q33L/E35D/T41S/M47V/ | 586 | 797 | 1008 |
| N48K/V68M/L85M | | | |
| Q33L/E35D/M47V/T62S/V68M/L85M | 587 | 798 | 1009 |
| Q33R/E35D/M38I/M47L/V68M | 588 | 799 | 1010 |
| R29C/E35D/M47L/V68M/A71G/L85M | 589 | 800 | 1011 |
| S21P/E35D/K37E/D46E/M47I/V68M | 590 | 801 | 1012 |
| S21P/E35D/K37E/D46E/M47I/V68M/R94L | 591 | 802 | 1013 |
| T13R/E35D/M47L/V68M | 592 | 803 | 1014 |
| T13R/H18Y/E35D/V68M/L85M/R94Q | 593 | 804 | 1015 |
| T13R/Q27L/Q33L/E35D/T41S/ | 594 | 805 | 1016 |
| M47V/N48K/V68M/L85M | | | |
| T13R/Q33L/E35D/M47L/V68M/L85M | 595 | 806 | 1017 |
| T13R/Q33L/E35D/M47V/T62S/V68M/L85M | 596 | 807 | 1018 |
| T13R/Q33R/E35D/M38I/M47L/V68M | 597 | 808 | 1019 |
| T13R/Q33R/E35D/M38I/M47L/ | 598 | 809 | 1020 |
| V68M/E95V/L97Q | | | |
| T13R/Q33R/E35D/M38I/M47L/V68M/L85M | 599 | 810 | 1021 |
| T13R/Q33R/E35D/M38I/M47L/ | 600 | 811 | 1022 |
| V68M/L85M/R94Q | | | |
| T13R/Q33R/E35D/M47L/V68M | 601 | 812 | 1023 |
| T13R/Q33R/E35D/M47L/V68M/L85M | 602 | 813 | 1024 |
| V22D/E24D/E35D/M47L/V68M | 603 | 814 | 1025 |
| V22D/E24D/E35D/M47L/V68M/L85M/D90G | 604 | 815 | 1026 |
| V22D/E24D/E35D/M47V/V68M | 605 | 816 | 1027 |
| D46V | 606 | 817 | 1028 |
| M47L | 607 | 818 | 1029 |
| V68M | 608 | 819 | 1030 |
| L85Q | 609 | 820 | 1031 |
| E35D/D46V | 610 | 821 | 1032 |
| E35D/V68M | 611 | 822 | 1033 |
| E35D/L85Q | 612 | 823 | 1034 |
| D46V/M47L | 613 | 824 | 1035 |
| D46V/V68M | 614 | 825 | 1036 |
| D46V/L85Q | 615 | 826 | 1037 |
| M47L/V68M | 616 | 827 | 1038 |
| M47L/L85Q | 617 | 828 | 1039 |
| V68M/L85Q | 618 | 829 | 1040 |
| E35D/D46V/M47L | 619 | 830 | 1041 |
| E35D/D46V/V68M | 620 | 831 | 1042 |
| E35D/D46V/L85Q | 621 | 832 | 1043 |
| E35D/V68M/L85Q | 622 | 833 | 1044 |
| D46V/M47L/V68M | 623 | 834 | 1045 |
| D46V/M47L/L85Q | 624 | 835 | 1046 |
| D46V/V68M/L85Q | 625 | 836 | 1047 |
| M47L/V68M/L85Q | 626 | 837 | 1048 |
| E35D/D46V/M47L/L85Q | 627 | 838 | 1049 |
| E35D/D46V/V68M/L85Q | 628 | 839 | 1050 |
| E35D/M47L/V68M/L85Q | 629 | 840 | 1051 |
| D46V/M47L/V68M/L85Q | 630 | 841 | 1052 |
| M47V | 631 | 842 | 1053 |
| N48K | 632 | 843 | 1054 |
| K89N | 633 | 844 | 1055 |
| E35D/N48K | 634 | 845 | 1056 |
| E35D/K89N | 635 | 846 | 1057 |
| M47V/N48K | 636 | 847 | 1058 |
| M47V/V68M | 637 | 848 | 1059 |
| M47V/K89N | 638 | 849 | 1060 |
| N48K/V68M | 639 | 850 | 1061 |
| N48K/K89N | 640 | 851 | 1062 |
| V68M/K89N | 641 | 852 | 1063 |
| E35D/M47V/N48K | 642 | 853 | 1064 |
| E35D/M47V/V68M | 643 | 854 | 1065 |
| E35D/M47V/K89N | 644 | 855 | 1066 |
| E35D/N48K/V68M | 645 | 856 | 1067 |
| E35D/N48K/K89N | 646 | 857 | 1068 |
| E35D/V68M/K89N | 647 | 858 | 1069 |
| M47V/N48K/V68M | 648 | 859 | 1070 |
| M47V/N48K/K89N | 649 | 860 | 1071 |
| M47V/V68M/K89N | 650 | 861 | 1072 |
| N48K/V68M/K89N | 651 | 862 | 1073 |
| E35D/M47V/N48K/K89N | 652 | 863 | 1074 |
| E35D/M47V/V68M/K89N | 653 | 864 | 1075 |
| E35D/N48K/V68M/K89N | 654 | 865 | 1076 |
| M47V/N48K/V68M/K89N | 655 | 866 | 1077 |
| E35D/D46V/M47V/N48K/V68M | 656 | 867 | 1078 |
| E35D/D46V/M47V/V68M/L85Q | 657 | 868 | 1079 |
| E35D/D46V/M47V/V68M/K89N | 658 | 869 | 1080 |
| E35D/M47V/N48K/V68M/L85Q | 659 | 870 | 1081 |
| E35D/M47V/V68M/L85Q/K89N | 660 | 871 | 1082 |
| A26E/E35D/M47L/V68M/A71G/D90G | 661 | 872 | 1083 |
| H18Y/E35D/M47L/V68M/A71G/D90G | 662 | 873 | 1084 |
| H18Y/A26E/M47L/V68M/A71G/D90G | 663 | 874 | 1085 |
| H18Y/A26E/E35D/V68M/A71G/D90G | 664 | 875 | 1086 |
| H18Y/A26E/E35D/M47L/A71G/D90G | 665 | 876 | 1087 |
| H18Y/A26E/E35D/M47L/V68M/D90G | 666 | 877 | 1088 |
| H18Y/A26E/E35D/M47L/V68M/A71G | 667 | 878 | 1089 |
| E35D/M47L/V68M/A71G/D90G | 668 | 879 | 1090 |
| H18Y/M47L/V68M/A71G/D90G | 669 | 880 | 1091 |
| H18Y/A26E/V68M/A71G/D90G | 670 | 881 | 1092 |
| H18Y/A26E/E35D/A71G/D90G | 671 | 882 | 1093 |
| H18Y/A26E/E35D/M47L/D90G | 672 | 883 | 1094 |
| H18Y/A26E/E35D/M47L/V68M | 673 | 884 | 1095 |
| A26E/M47L/V68M/A71G/D90G | 674 | 885 | 1096 |
| A26E/E35D/V68M/A71G/D90G | 675 | 886 | 1097 |
| A26E/E35D/M47L/A71G/D90G | 676 | 887 | 1098 |
| A26E/E35D/M47L/V68M/D90G | 677 | 888 | 1099 |
| A26E/E35D/M47L/V68M/A71G | 678 | 889 | 1100 |
| H18Y/E35D/V68M/A71G/D90G | 679 | 890 | 1101 |
| H18Y/E35D/M47L/A71G/D90G | 680 | 891 | 1102 |
| H18Y/E35D/M47L/V68M/D90G | 681 | 892 | 1103 |
| H18Y/E35D/M47L/V68M/A71G | 682 | 893 | 1104 |
| H18Y/A26E/M47L/A71G/D90G | 683 | 894 | 1105 |
| H18Y/A26E/M47L/V68M/D90G | 684 | 895 | 1106 |
| H18Y/A26E/M47L/V68M/A71G | 685 | 896 | 1107 |
| H18Y/A26E/E35D/V68M/D90G | 686 | 897 | 1108 |
| H18Y/A26E/E35D/V68M/A71G | 687 | 898 | 1109 |
| H18Y/A26E/E35D/M47L/A71G | 688 | 899 | 1110 |
| M47L/V68M/A71G/D90G | 689 | 900 | 1111 |
| H18Y/V68M/A71G/D90G | 690 | 901 | 1112 |
| H18Y/A26E/A71G/D90G | 691 | 902 | 1113 |
| H18Y/A26E/E35D/D90G | 692 | 903 | 1114 |
| H18Y/A26E/E35D/M47L | 693 | 904 | 1115 |
| E35D/V68M/A71G/D90G | 694 | 905 | 1116 |
| E35D/M47L/A71G/D90G | 695 | 906 | 1117 |
| E35D/M47L/V68M/D90G | 696 | 907 | 1118 |
| E35D/M47L/V68M/A71G | 697 | 908 | 1119 |
| A26E/V68M/A71G/D90G | 698 | 909 | 1120 |
| A26E/M47L/A71G/D90G | 699 | 910 | 1121 |
| A26E/M47L/V68M/D90G | 700 | 911 | 1122 |
| A26E/M47L/V68M/A71G | 701 | 912 | 1123 |
| A26E/E35D/A71G/D90G | 702 | 913 | 1124 |
| A26E/E35D/V68M/D90G | 703 | 914 | 1125 |
| A26E/E35D/V68M/A71G | 704 | 915 | 1126 |
| A26E/E35D/M47L/D90G | 705 | 916 | 1127 |
| A26E/E35D/M47L/V68M | 706 | 917 | 1128 |
| H18Y/M47L/A71G/D90G | 707 | 918 | 1129 |
| H18Y/M47L/V68M/D90G | 708 | 919 | 1130 |
| H18Y/M47L/V68M/A71G | 709 | 920 | 1131 |
| H18Y/E35D/A71G/D90G | 710 | 921 | 1132 |
| H18Y/E35D/V68M/D90G | 711 | 922 | 1133 |
| H18Y/E35D/V68M/A71G | 712 | 923 | 1134 |
| H18Y/E35D/M47L/D90G | 713 | 924 | 1135 |
| H18Y/E35D/M47L/A71G | 714 | 925 | 1136 |
| H18Y/E35D/M47L/V68M | 715 | 926 | 1137 |
| H18Y/A26E/V68M/D90G | 716 | 927 | 1138 |
| H18Y/A26E/V68M/A71G | 717 | 928 | 1139 |
| H18Y/A26E/M47L/D90G | 718 | 929 | 1140 |
| H18Y/A26E/M47L/A71G | 719 | 930 | 1141 |
| H18Y/A26E/M47L/V68M | 720 | 931 | 1142 |
| H18Y/A26E/E35D/A71G | 721 | 932 | 1143 |
| H18Y/A26E/E35D/V68M | 722 | 933 | 1144 |
| H18Y/E35D/M47V/V68M/A71G | 1145 | 1176 | 1207 |
| H18C/A26P/E35D/M47L/V68M/A71G | 1146 | 1177 | 1208 |
| H18I/A26P/E35D/M47V/V68M/A71G | 1147 | 1178 | 1209 |
| H18L/A26N/D46E/V68M/A71G/D90G | 1148 | 1179 | 1210 |
| H18L/E35D/M47V/V68M/A71G/D90G | 1149 | 1180 | 1211 |
| H18T/A26N/E35D/M47L/V68M/A71G | 1150 | 1181 | 1212 |
| H18V/A26K/E35D/M47L/V68M/A71G | 1151 | 1182 | 1213 |
| H18V/A26N/E35D/M47V/V68M/A71G | 1152 | 1183 | 1214 |
| H18V/A26P/E35D/M47V/V68L/A71G | 1153 | 1184 | 1215 |
| H18V/A26P/E35D/M47L/V68M/A71G | 1154 | 1185 | 1216 |
| H18V/E35D/M47V/V68M/A71G/D90G | 1155 | 1186 | 1217 |
| H18Y/A26P/E35D/M47I/V68M/A71G | 1156 | 1187 | 1218 |
| H18Y/A26P/E35D/M47V/V68M/A71G | 1157 | 1188 | 1219 |
| H18Y/E35D/M47V/V68L/A71G/D90G | 1158 | 1189 | 1220 |
| H18Y/E35D/M47V/V68M/A71G/D90G | 1159 | 1190 | 1221 |
| A26P/E35D/M47I/V68M/A71G/D90G | 1160 | 1191 | 1222 |
| H18V/A26G/E35D/M47V/V68M/A71G/D90G | 1161 | 1192 | 1223 |
| H18V/A26S/E35D/M47L/V68M/A71G/D90G | 1162 | 1193 | 1224 |
| H18V/A26R/E35D/M47L/V68M/A71G/D90G | 1163 | 1194 | 1225 |
| H18V/A26D/E35D/M47V/V68M/A71G/D90G | 1164 | 1195 | 1226 |
| H18V/A26Q/E35D/M47V/V68L/A71G/D90G | 1165 | 1196 | 1227 |
| H18A/A26P/E35D/M47L/V68M/A71G/D90G | 1166 | 1197 | 1228 |
| H18A/A26N/E35D/M47L/V68M/A71G/D90G | 1167 | 1198 | 1229 |
| H18F/A26P/E35D/M47I/V68M/A71G/D90G | 1168 | 1199 | 1230 |
| H18F/A26H/E35D/M47L/V68M/A71G/D90G | 1169 | 1200 | 1231 |
| H18F/A26N/E35D/M47V/V68M/A71G/D90K | 1170 | 1201 | 1232 |
| H18Y/A26N/E35D/M47F/V68M/A71G/D90G | 1171 | 1202 | 1233 |
| H18Y/A26P/E35D/M47Y/V68I/A71G/D90G | 1172 | 1203 | 1234 |
| H18Y/A26Q/E35D/M47T/V68M/A71G/D90G | 1173 | 1204 | 1235 |
| H18R/A26P/E35D/D46N/M47V/ | 1174 | 1205 | 1236 |
| V68M/A71G/D90P | | | |
| H18F/A26D/E35D/D46E/M47T/ | 1175 | 1206 | 1237 |
| V68M/A71G/D90G | | | |
| L70Q/A91G | 1447 | 1256 | 1370 |
| L70Q/A91G/T130A | 1448 | | |
| L70Q/A91G/I118A/T120S/T130A | 1449 | | |
| V4M/L70Q/A91G/T120S/T130A | 1450 | 1257 | |
| L70Q/A91G/T120S/T130A | 1451 | | |
| V20L/L70Q/A91S/T120S/T130A | 1452 | 1258 | |
| S44P/L70Q/A91G/T130A | 1453 | 1259 | |
| L70Q/A91G/E117G/T120S/T130A | 1454 | | |
| A91G/T120S/T130A | 1455 | 1260 | |
| L70R/A91G/T120S/T130A | 1456 | 1261 | |
| L70Q/E81A/A91G/T120S/I127T/T130A | 1457 | 1262 | |
| L70Q/Y87N/A91G/T130A | 1458 | 1263 | |
| T28S/L70Q/A91G/E95K/T120S/T130A | 1459 | 1264 | |
| N63S/L70Q/A91G/T120S/T130A | 1460 | 1265 | |
| K36E/I67T/L70Q/A91G/T120S/T130A/N152T | 1461 | 1266 | |
| E52G/L70Q/A91G/T120S/T130A | 1462 | 1267 | |
| K37E/F59S/L70Q/A91G/T120S/T130A | 1463 | 1268 | |
| A91G/S103P | 1464 | | 1368 |
| K89E/T130A | 1465 | 1269 | 1381 |
| A91G | 1466 | 1257 | 1371 |
| D60V/A91G/T120S/T130A | 1467 | 1270 | |
| K54M/A91G/T120S | 1468 | 1271 | |
| M38T/L70Q/E77G/A91G/T120S/T130A/N152T | 1469 | 1272 | |
| R29H/E52G/L70R/E88G/A91G/T130A | 1470 | 1273 | |
| Y31H/T41G/L70Q/A91G/T120S/T130A | 1471 | 1274 | |
| V68A/T110A | 1472 | 1275 | |
| S66H/D90G/T110A/F116L | 1473 | 1276 | |
| R29H/E52G/T120S/T130A | 1474 | 1277 | |
| A91G/L102S | 1475 | | 1382 |
| I67T/L70Q/A91G/T120S | 1476 | 1278 | |
| L70Q/A91G/T110A/T120S/T130A | 1477 | | |
| M38V/T41D/M43I/W50G/D76G/ | 1478 | 1279 | |
| V83A/K89E/T120S/T130A | | | |
| V22A/L70Q/S121P | 1479 | 1280 | |
| A12V/S15F/Y31H/T41G/T130A/P137L/N152T | 1480 | 1281 | |
| I67F/L70R/E88G/A91G/T120S/T130A | 1481 | 1282 | |
| E24G/L25P/L70Q/T120S | 1482 | 1283 | |
| A91G/F92L/F108L/T120S | 1483 | 1284 | |
| R29D/Y31L/Q33H/K36G/M38I/ | 1484 | 1285 | |
| T41A/M43R/M47T/E81V/ | | | |
| L85R/K89N/A91T/F92P/K93V/ | | | |
| R94L/I118T/N149S | | | |
| R29D/Y31L/Q33H/K36G/M38I/ | 1485 | | |
| T41A/M43R/M47T/E81V/ | | | |
| L85R/K89N/A91T/F92P/K93V/ | | | |
| R94L/N144S/N149S | | | |
| R29D/Y31L/Q33H/K36G/M38I/ | 1486 | 1286 | |
| T41A/M42T/M43R/M47T/ | | | |
| E81V/L85R/K89N/A91T/F92P/ | | | |
| K93V/R94L/L148S/N149S | | | |
| E24G/R29D/Y31L/Q33H/K36G/ | 1487 | 1287 | |
| M38I/T41A/M43R/M47T/ | | | |
| F59L/E81V/L85R/K89N/A91T/ | | | |
| F92P/K93V/R94L/H96R/ | | | |
| N149S/C182S | | | |
| R29D/Y31L/Q33H/K36G/M38I/ | 1488 | | |
| T41A/M43R/M47T/E81V/ | | | |
| L85R/K89N/A91T/F92P/K93V/R94L/N149S | | | |
| R29V/M43Q/E81R/L85I/K89R/ | 1489 | 1288 | 1372 |
| D90L/A91E/F92N/K93Q/ | | | |
| R94G | | | |
| T41I/A91G | 1490 | 1289 | 1373 |
| K89R/D90K/A91G/F92Y/K93R/N122S/N177S | 1491 | 1290 | |
| K89R/D90K/A91G/F92Y/K93R | 1492 | 1367 | 1374 |
| K36G/K37Q/M38I/F59L/E81V/ | 1493 | 1291 | |
| L85R/K89N/A91T/F92P/ | | | |
| K93V/R94L/E99G/T130A/N149S | | | |
| E88D/K89R/D90K/A91G/F92Y/K93R | 1494 | 1292 | 1375 |
| K36G/K37Q/M38I/L40M | 1495 | 1293 | 1376 |
| K36G | 1496 | 1294 | 1377 |
| R29H/Y31H/T41G/Y87N/E88G/ | 1497 | 1295 | |
| K89E/D90N/A91G/P109S | | | |
| A12T/H18L/M43V/F59L/E77K/P109S/I118T | 1498 | 1296 | |
| R29V/Y31F/K36G/M38L/ | 1499 | 1297 | 1378 |
| M43Q/E81R/V83I/L85I/K89R/ | | | |
| D90L/A91E/F92N/K93Q/R94G | | | |
| V68M/L70P/L72P/K86E | 1500 | 1298 | 1379 |
| R29D/Y31L/Q33H/K36G/M38I/ | 1299 | | |
| T41A/M43R/M47T/E81V/ | | | |
| L85R/K89N/A91T/F92P/K93V/ | | | |
| R94L/T120S/I127T/T130A/ | | | |
| K169E | | | |
| R29D/Y31L/Q33H/K36G/M38I/ | 1300 | | |
| T41A/M43R/M47T/L70Q/ | | | |
| E81V/L85R/K89N/A91T/F92P/ | | | |
| K93V/R94L/T120S/I127T/T130A | | | |
| H18L/R29D/Y31L/Q33H/K36G/ | 1301 | | |
| M38I/T41A/M43R/M47T/ | | | |
| E81V/L85R/K89N/A91T/F92P/ | | | |
| K93V/R94L/T120S/I127T/ | | | |
| T130A/K169E | | | |
| R29D/Y31L/Q33H/K36G/M38I/ | 1302 | | |
| T41A/M43R/M47T/E81V/ | | | |
| L85R/K89N/A91T/F92P/K93V/ | | | |
| R94L/T120S/T130A/M174T | | | |
| R29D/Y31L/Q33H/K36G/M38I/ | 1303 | | |
| T41A/M43R/M47T/F59L/ | | | |
| E81V/L85R/K89N/A91T/F92P/ | | | |
| K93V/R94L/T120S/I127T/ | | | |
| T130A/H188D | | | |
| H18R/R29D/Y31L/Q33H/K36G/ | 1304 | | |
| K37E/M38I/T41A/M43R/ | | | |
| M47T/L70Q/E81V/L85R/K89N/ | | | |
| A91T/F92P/K93V/R94L/T120S/ | | | |
| T130A/K169E/H188D | | | |
| R29D/Y31L/Q33H/K36G/M38I/ | 1305 | | |
| T41A/M43R/M47T/L70Q/ | | | |
| E81V/L85R/K89N/A91T/F92P/ | | | |
| K93V/R94L/T120S/I127T/ | | | |
| T130A/E143G/K169E/M174V/H188D | | | |
| R29D/Y31L/Q33H/K36G/M38I/ | 1306 | | |
| T41A/M43R/M47T/L70Q/ | | | |
| E81V/L85R/K89N/A91T/F92P/ | | | |
| K93V/R94L/T120S/I127T/T130A | | | |
| R29D/Y31L/Q33H/K36G/M38I/ | 1307 | | |
| T41A/M43R/M47T/E81V/ | | | |
| L85R/K89N/A91T/F92P/K93V/ | | | |
| R94L/T120S/I127T/T130A/ | | | |
| H188D | | | |
| R29D/Y31L/Q33H/K36G/M38I/ | 1308 | | |
| T41A/M43R/M47T/L70Q/ | | | |
| E81V/L85R/K89N/A91T/F92P/ | | | |
| K93V/R94L/T120S/I127T/ | | | |
| T130A/K169E | | | |
| R29D/Y31L/Q33H/K36G/M38I/ | 1309 | | |
| T41A/M43R/M47T/L70Q/ | | | |
| E81V/L85R/K89N/A91T/F92P/ | | | |
| K93V/R94L/T120S/I127T/T130A | | | |
| R29D/Y31L/Q33H/K36G/M38I/ | 1310 | | |
| T41A/M43R/M47T/L85R/ | | | |
| K89N/A91T/F92P/K93V/R94L/ | | | |
| T120S/I127T/T130A/K169E/ | | | |
| H188D | | | |
| R29D/Y31L/Q33H/K36G/M38I/ | 1311 | | |
| T41A/M43R/M47T/E81V/ | | | |
| L85R/K89N/A91T/F92P/K93V/ | | | |
| R94L/F108L/T120S/T130A/ | | | |
| K169E/H188D | | | |
| R29D/Y31L/Q33H/K36G/M38I/ | 1312 | | |
| T41A/M43R/M47T/L70Q/ | | | |
| E81V/L85R/K89N/A91T/F92P/ | | | |
| K93V/R94L/T130A/H188D | | | |
| R29D/Y31L/Q33H/K36G/M38I/ | 1313 | | |
| T41A/M43R/M47T/L70Q/ | | | |
| E81V/L85R/K89N/A91T/F92P/ | | | |
| K93V/R94L/T120S/I127T/ | | | |
| T130A/K169E | | | |
| H18L/R29D/Y31L/Q33H/K36G/ | 1314 | | |
| M38I/T41A/M43R/M47T/ | | | |
| L70Q/E81V/L85R/K89N/A91T/ | | | |
| F92P/K93V/R94L/T120S/ | | | |
| T130A/K169E/H188D | | | |
| R29D/Y31L/Q33H/K36G/M38I/ | 1315 | | |
| T41A/M43R/M47T/E81V/ | | | |
| L85R/K89N/A91T/F92P/K93V/ | | | |
| R94L/T120S/I127T/C128Y/ | | | |
| T130A/H188D | | | |
| R29D/Y31L/Q33H/K36G/M38I/ | 1316 | | |
| T41A/M43R/M47T/E81V/ | | | |
| L85R/K89N/A91T/F92P/K93V/ | | | |
| R94F/T130A/K169E | | | |
| H18L/R29D/Y31L/Q33H/K36G/ | 1317 | | |
| M38I/T41A/M43R/M47T/ | | | |
| E81V/L85R/K89N/A91T/F92P/ | | | |
| K93V/R94L/T130A | | | |
| H18L/R29D/Y31L/Q33H/K36G/ | 1318 | | |
| M38I/T41A/M43R/M47T/ | | | |
| L70Q/E81V/L85R/K89N/A91T/ | | | |
| F92P/K93V/R94L/T120S/ | | | |
| T130A/K169E | | | |
| R29D/Y31L/Q33H/K36G/M38I/ | 1319 | | |
| T41A/M43R/M47T/L70Q/ | | | |
| E81V/L85R/K89N/A91T/F92P/ | | | |
| K93I/R94L/L97R/T130A | | | |
| R29D/Y31L/Q33H/K36G/M38I/ | 1320 | | |
| T41A/M43R/M47T/L70Q/ | | | |
| E81V/L85R/K89N/A91T/F92P/ | | | |
| K93I/R94L/L97R/T130A/L148S | | | |
| H18L/R29D/Y31L/Q33H/K36G/ | 1321 | | |
| M38I/T41A/M43R/M47T/ | | | |
| E81V/L85R/K89N/A91T/F92P/ | | | |
| K93V/R94L/I118V/T120S/I127T/ | | | |
| T130A/K169E | | | |
| R29D/Y31L/Q33H/K36G/M38I/ | 1322 | | |
| T41A/M43R/M47T/I61N/ | | | |
| E81V/L85R/K89N/A91T/F92P/ | | | |
| K93V/R94F/V104A/T120S/T130A | | | |
| R29D/Y31L/Q33H/K36G/M38I/ | 1323 | | |
| T41A/M43R/M47T/E81V/ | | | |
| L85R/K89N/F92P/K93V/R94F/ | | | |
| I118V/T130A | | | |
| R29D/Y31L/Q33H/K36G/M38I/ | 1324 | | |
| T41A/M43R/M47T/T62S/ | | | |
| E81V/L85R/K89N/A91T/F92P/ | | | |
| K93V/R94L/I118V/T120S/T130A/ | | | |
| K169E/T175A | | | |
| H18L/R29D/Y31L/Q33H/K36G/ | 1325 | | |
| M38I/T41A/M43R/M47T/ | | | |
| E81V/L85R/K89N/A91T/F92P/ | | | |
| K93V/R94L/F116S/T130A/ | | | |
| H188D | | | |
| H18L/R29D/Y31L/Q33H/K36G/ | 1326 | | |
| M38I/T41A/M43R/M47T/ | | | |
| E81V/L85R/K89N/A91T/F92P/ | | | |
| K93V/R94L/T120S/I127T/ | | | |
| T130A/L142S/H188D | | | |
| C16S/H18L/R29D/Y31L/Q33H/ | 1327 | | |
| K36G/M38I/T41A/M43R/ | | | |
| M47T/E81V/L85R/K89N/A91T/ | | | |
| F92P/K93V/R94L/T110A/ | | | |
| H188D | | | |
| R29D/Y31L/Q33H/K36G/M38I/ | 1328 | | |
| T41A/M43R/M47T/A91G/ | | | |
| T120S/I127T/T130A/H188D | | | |
| R29D/Y31L/Q33H/K36G/M38I/ | 1329 | | |
| T41A/M43R/M47T/L70Q/ | | | |
| D76G/A91G/S103L/T120S/I127T/T130A | | | |
| DELTAQ33/Y53C/L85R/K89N/ | 1330 | | |
| A91T/F92P/K93V/R94L/ | | | |
| T120S/I127T/T130A/K169E | | | |
| T62S/E81V/L85R/K89N/A91T/ | 1331 | | |
| F92P/K93V/R94L/T120S/ | | | |
| T130A/K169E | | | |
| R29D/Y31L/Q33H/K36G/M38I/ | 1332 | | |
| T41A/M43R/M47T/E81V/ | | | |
| L85R/K89N/A91T/F92P/K93V/ | | | |
| R94L/S129L/H188D | | | |
| K9E/E10R/V11S/A12G/T13N/ | 1333 | | |
| L14A/S15V/C16L/G17W/ | | | |
| H18Y/Y53C/L70Q/D90G/T130A/ | | | |
| N149D/N152T/H188D | | | |
| H18L/R29D/Y31L/Q33H/K36G/ | 1334 | | |
| T41A/M43R/M47T/E81V/ | | | |
| L85R/K89N/A91T/F92P/K93V/ | | | |
| R94L/I118V/T120S/I127T/ | | | |
| T130A/H188D | | | |
| K89E/K93E/T130A | 1335 | | |
| S21P/R29D/Y31L/Q33H/K36G/ | 1336 | | |
| M38I/T41A/M43R/M47T/ | | | |
| N48I/V68A/E81V/L85R/K89N/ | | | |
| A91T/F92P/K93V/R94L/ | | | |
| P109H/I126L/K169I | | | |
| H18L/R29D/Y31L/Q33H/K36G/ | 1337 | 1366 | 1380 |
| M38I/T41A/M43R/M47T/ | | | |
| P74L/Y80N/E81V/L85R/K89N/ | | | |
| A91T/F92P/K93V/R94L/ | | | |
| L97R | | | |
| S21P/R29D/Y31L/Q33H/K36G/ | 1338 | | |
| M38I/T41A/M43R/M47T/ | | | |
| P74L/Y80N/E81V/L85R/K89N/ | | | |
| D90N/A91T/F92P/K93V/R94L/ | | | |
| T130A/N149S/E162G | | | |
| H18L/R29D/Y31L/Q33H/K36G/ | 1339 | | |
| M38I/T41A/M43R/M47T/ | | | |
| V68M/E81V/L85R/K89N/A91T/ | | | |
| F92P/K93V/R94L/T130A | | | |
| R29D/Y31L/Q33H/K36G/M38I/ | 1340 | | |
| T41A/M43R/M47T/V68M/ | | | |
| E81V/L85R/K89N/A91T/F92P/ | | | |
| K93V/R94L/T130A/N149S/ | | | |
| R190S | | | |
| H18L/R29D/Y31L/Q33H/K36G/ | 1341 | | |
| M38I/T41A/M43R/M47T/ | | | |
| P74L/Y80N/E81V/L85R/K89N/ | | | |
| A91T/F92P/K93V/R94L/ | | | |
| T130A/R190S | | | |
| C16G/V22A/R29D/Y31L/Q33H/ | 1342 | | |
| K36G/M38I/T41A/M43R/ | | | |
| M47T/V68M/D76G/E81V/L85R/ | | | |
| K89N/A91T/F92P/K93V/R94L/ | | | |
| I118T/T130A/S140T/N149S/ | | | |
| K169I/H178R/N192D | | | |
| R29D/Y31L/Q33H/K36G/M38I/ | 1343 | | |
| T41A/M43R/M47T/E81V/ | | | |
| L85R/K89N/A91T/F92P/K93V/ | | | |
| R94F/E117V/I118T/N149S/ | | | |
| S168G/H188Q | | | |
| V22A/R29D/Y31L/Q33H/K36G/ | 1344 | | |
| M38I/T41A/M43R/M47T/ | | | |
| V68M/E81V/L85R/K89N/A91T/ | | | |
| F92P/K93V/R94L/T130A | | | |
| R29D/Y31L/Q33H/K36G/M38I/ | 1345 | | |
| T41A/M43R/M47T/N64S/ | | | |
| E81V/L85R/K89N/A91T/F92P/K93V/ | | | |
| R94F/I118T/T130A/N149S/K169I | | | |
| V22A/R29D/Y31L/Q33H/K36G/ | 1346 | | |
| M38I/T41A/M43R/M47T/ | | | |
| V68M/E81V/L85R/K89N/A91T/ | | | |
| F92P/K93V/R94L/D115G/ | | | |
| I118T/T130A/G133D/N149S | | | |
| S129P | 1347 | | |
| A91G/S129P | 1348 | | |
| I69T/L70Q/A91G/T120S | 1349 | | |
| Y31H/S129P | 1350 | | |
| T28A/R29D/Y31L/Q33H/K36G/ | 1351 | | |
| M38I/T41A/M43R/M47T/ | | | |
| E81V/L85R/K89N/A91T/F92P/K93V/R94L/ | | | |
| V104L/T130A/N149S | | | |
| H18L/R29D/Y31L/Q33H/K36G/ | 1352 | | |
| M38I/T41A/M43R/M47T/ | | | |
| E81V/L85R/K89N/A91T/F92P/ | | | |
| K93V/R94L/L97R/N149S/ | | | |
| H188Q | | | |
| H18L/R29D/Y31L/Q33H/K36G/ | 1353 | | |
| M38I/T41A/M43R/M47T/ | | | |
| E81V/L85R/K89N/A91T/F92P/ | | | |
| K93V/R94L/L97R/N149S | | | |
| H18L/R29D/Y31L/Q33H/K36G/ | 1354 | | |
| M38I/T41A/M43R/M47T/ | | | |
| V68A/E81V/L85R/K89N/A91T/ | | | |
| F92P/K93V/R94L/ | | | |
| T130A/N149S/T154I | | | |
| A12G/R29D/Y31L/Q33H/K36G/ | 1355 | | |
| M38I/T41A/M43R/M47T/ | | | |
| V68A/E81V/L85R/K89N/A91T/ | | | |
| F92P/K93V/R94L/ | | | |
| L97R/T130A/L183H | | | |
| R29D/Y31L/Q33H/K36G/M38I/ | 1356 | | |
| T41A/M43R/M47T/E81V/ | | | |
| L85R/K89N/A91T/F92P/K93V/ | | | |
| R94L/I118T/T130A/S140T/ | | | |
| N149S/K169S | | | |
| R29D/Y31L/Q33H/K36G/M38I/ | 1357 | | |
| T41A/M43R/M47T/E81V/ | | | |
| L85R/K89N/A91T/F92P/K93V/ | | | |
| R94L/I118T/T130A/N149S/ | | | |
| K169I/Q193L | | | |
| V22A/R29D/Y31L/Q33H/K36G/ | 1358 | | |
| M38I/T41A/M43R/M47T/ | | | |
| E81V/L85R/K89N/A91T/F92P/K93V/ | | | |
| R94L/I118T/T130A/N149S | | | |
| R29D/Y31L/Q33H/K36G/M38I/ | 1359 | | |
| T41A/M43R/M47T/E81V/ | | | |
| L85R/K89N/A91T/F92P/K93V/R94L/ | | | |
| I118T/T130A/N149S | | | |
| R29D/Y31L/Q33H/K36G/M38I/T41A/ | 1360 | | |
| M43R/M47T/E81V/ | | | |
| L85R/K89N/A91T/F92P/K93V/ | | | |
| R94L/I118T/T130A/N149S/ | | | |
| K169I | | | |
| R29D/Y31L/Q33H/K36G/M38I/ | 1361 | | |
| T41A/M43R/M47T/E81V/ | | | |
| L85R/K89N/A91T/F92P/K93V/ | | | |
| R94F/T130A/N149S/K169I | | | |
| I118T/C128R | 1362 | | |
| Q27R/R29C/M42T/S129P/E160G | 1363 | | |
| S129P/T154A | 1364 | | |
| S21P/L70Q/D90G/T120S/T130A | 1365 | | |
| L70Q/A91G/N144D | 1383 | | |
| L70Q/A91G/I118A/T120S/T130A/K169E | 1384 | | |
| V4M/L70Q/A91G/I118V/T120S/T130A/K169E | 1385 | | |
| L70Q/A91G/I118V/T120S/T130A/K169E | 1386 | | |
| L70Q/A91G/I118V/T120S/T130A | 1387 | | |
| V20L/L70Q/A91S/I118V/T120S/T130A | 1388 | | |
| L70Q/A91G/E117G/I118V/T120S/T130A | 1389 | | |
| A91G/I118V/T120S/T130A | 1390 | | |
| L70R/A91G/I118V/T120S/T130A/T199S | 1391 | | |
| L70Q/E81A/A91G/I118V/T120S/I127T/T130A | 1392 | | |
| T28S/L70Q/A91G/E95K/I118V/ | 1393 | | |
| T120S/I126V/T130A/K169E | | | |
| N63S/L70Q/A91G/S114T/I118V/T120S/T130A | 1394 | | |
| K36E/I67T/L70Q/A91G/I118V/ | 1395 | | |
| T120S/T130A/N152T | | | |
| E52G/L70Q/A91G/D107N/I118V/ | 1396 | | |
| T120S/T130A/K169E | | | |
| K37E/F59S/L70Q/A91G/I118V/ | 1397 | | |
| T120S/T130A/K185E | | | |
| D60V/A91G/I118V/T120S/T130AK169E | 1398 | | |
| K54M/L70Q/A91G/Y164H/T120S | 1399 | | |
| M38T/L70Q/E77G/A91G/I118V/ | 1400 | | |
| T120S/T130A/N152T | | | |
| Y31H/T41G/M43L/L70Q/A91G/ | 1401 | | |
| I118V/T120S/I126V/T130A | | | |
| L65H/D90G/T110A/F116L | 1402 | | |
| R29H/E52G/D90N/I118V/T120S/T130A | 1403 | | |
| 167T/L70Q/A91G/I118V/T120S | 1405 | | |
| L70Q/A91G/T110A/I118V/T120S/T130A | 1406 | | |
| M38V/T41D/M43I/W50G/D76G/ | 1407 | | |
| V83A/K89E/I118V/T120S/ | | | |
| I126V/T130A | | | |
| A12V/S15F/Y31H/M38L/T41G/ | 1408 | | |
| M43L/D90N/T130A/P137L/ | | | |
| N149D/N152T | | | |
| I67F/L70R/E88G/A91G/I118V/T120S/T130A | 1409 | | |
| E24G/L25P/L70Q/A91G/I118V/T120S/N152T | 1410 | | |
| A91G/F92L/F108L/I118V/T120S | 1411 | | |
| E88D/K89R/D90K/A91G/F92Y/ | 1412 | | |
| K93R/N122S/N177S | | | |
| K36G/K37Q/M38I/L40M/F59L/ | 1413 | | |
| E81V/L85R/K89N/A91T/ | | | |
| F92P/K93V/R94L/E99G/T130A/N149S | | | |
| K36G/L40M | 1414 | 1445 | 1446 |
| R29D/Y31L/Q33H/K36G/M38I/T41A/ | 1415 | | |
| M43R/M47T/E81V/ | | | |
| L85R/K89N/A91T/F92P/K93V/ | | | |
| R94L/I118V/T120S/I127T/ | | | |
| T130A/K169E | | | |
| R29D/Y31L/Q33H/K36G/M38I/ | 1416 | | |
| T41A/M43R/M47T/L70Q/ | | | |
| E81V/L85R/K89N/A91T/F92P/ | | | |
| K93V/R94L/I118V/T120S/I127T/ | | | |
| T130A | | | |
| H18L/R29D/Y31L/Q33H/K36G/ | 1417 | | |
| M38I/T41A/M43R/M47T/ | | | |
| E81V/L85R/K89N/A91T/F92P/ | | | |
| K93V/R94L/I118V/T120S/I127T/ | | | |
| T130A/K169E | | | |
| R29D/Y31L/Q33H/K36G/M38I/ | 1418 | | |
| T41A/M43R/M47T/E81V/ | | | |
| L85R/K89N/A91T/F92P/K93V/ | | | |
| R94L/I118V/T120S/T130A/ | | | |
| K169E/M174T | | | |
| R29D/Y31L/Q33H/K36G/M38I/ | 1419 | | |
| T41A/M43R/M47T/N48D/ | | | |
| F59L/E81V/L85R/K89N/A91T/ | | | |
| F92P/K93V/R94L/I118V/T120S/ | | | |
| I127T/T130A/H188D | | | |
| H18R/R29D/Y31L/Q33H/K36G/ | 1420 | | |
| K37E/M38I/T41A/M43R/ | | | |
| M47T/L70Q/E81V/L85R/K89N/ | | | |
| A91T/F92P/K93V/R94L/I118V/ | | | |
| T120S/T130A/K169E/H188D | | | |
| R29D/Y31L/Q33H/K36G/M38I/ | 1421 | | |
| T41A/M43R/M47T/L70Q/ | | | |
| E81V/L85R/K89N/A91T/F92P/K93V/ | | | |
| R94L/I118V/T120S/I127T/ | | | |
| T130A/E143G/K169E/M174V/H188D | | | |
| R29D/I30V/Y31L/Q33H/K36G/ | 1422 | | |
| M38I/T41A/M43R/M47T/ | | | |
| E81V/L85R/K89N/A91T/F92P/ | | | |
| K93V/R94L/I118V/T120S/I127T/ | | | |
| T130A/H188D | | | |
| R29D/Y31L/Q33H/K36G/M38I/ | 1423 | | |
| T41A/M43R/M47T/E81V/ | | | |
| L85R/K89N/A91T/F92P/K93V/ | | | |
| R94L/I118V/T120S/I127T/ | | | |
| T130A/H188D | | | |
| R29D/Y31L/Q33H/K36G/M38I/ | 1424 | | |
| T41A/M43R/M47T/L70Q/ | | | |
| E81V/L85R/K89N/A91T/F92P/K93V/R94L/ | | | |
| I118V/T120S/I127T/T130A/K169E | | | |
| R29D/Y31L/Q33H/K36G/M38I/ | 1425 | | |
| T41A/M43R/M47T/L70Q/ | | | |
| E81V/K89N/A91T/F92P/K93V/ | | | |
| R94L/I118V/T120S/I127T/ | | | |
| T130A | | | |
| R29D/Y31L/Q33H/K36G/M38I/ | 1426 | | |
| T41A/M43R/M47T/L85R/ | | | |
| K89N/A91T/F92P/K93V/R94L/ | | | |
| I118V/T120S/I127T/T130A/ | | | |
| K169E/H188D | | | |
| R29D/Y31L/Q33H/K36G/M38I/ | 1427 | | |
| T41A/M43R/M47T/E81V/ | | | |
| L85R/K89N/A91T/F92P/K93V/ | | | |
| R94L/F108L/I118V/T120S/ | | | |
| T130A/K169E/H188D | | | |
| R29D/Y31L/Q33H/K36G/M38I/ | 1428 | | |
| T41A/M43R/M47T/L70Q/ | | | |
| E81V/L85R/K89N/A91T/F92P/ | | | |
| K93V/R94L/I118V/T120S/ | | | |
| T130A/N149D/K169E/H188D | | | |
| H18L/R29D/Y31L/Q33H/K36G/ | 1429 | | |
| M38I/T41A/M43R/M47T/ | | | |
| L70Q/E81V/L85R/K89N/ | | | |
| A91T/F92P/K93V/R94L/ | | | |
| I118V/T120S/T130A/K169E/H188D | | | |
| R29D/Y31L/Q33H/K36G/M38I/ | 1430 | | |
| T41A/M43R/M47T/E81V/ | | | |
| L85R/K89N/A91T/F92P/K93V/ | | | |
| R94L/I118V/T120S/I127T/ | | | |
| C128Y/T130A/H188D | | | |
| H18L/R29D/Y31L/Q33H/K36G/ | 1431 | | |
| M38I/T41A/M43R/M47T/ | | | |
| E81V/L85R/K89N/A91T/F92P/ | | | |
| K93V/R94L/E99D/T130A | | | |
| H18L/R29D/Y31L/Q33H/K36G/ | 1432 | | |
| M38I/T41A/M43R/M47T/ | | | |
| L70Q/E81V/L85R/K89N/ | | | |
| A91T/F92P/K93V/R94L/ | | | |
| I118V/T120S/T130A/K169E | | | |
| R29D/Y31L/Q33H/K36G/M38I/ | 1433 | | |
| T41A/M43R/M47T/I61N/ | | | |
| E81V/L85R/K89N/A91T/F92P/ | | | |
| K93V/R94F/V104A/ | | | |
| I118V/T120S/I126V/T130A | | | |
| R29D/Y31L/Q33H/K36G/M38I/ | 1434 | | |
| T41A/M43R/M47T/E81V/ | | | |
| L85R/K89N/A91T/F92P/K93V/ | | | |
| R94F/I118V/T120S/T130A | | | |
| R29D/Y31L/Q33H/K36G/M38I/ | 1435 | | |
| T41A/M43R/M47T/T62S/ | | | |
| E81V/L85R/K89N/A91T/ | | | |
| F92P/K93V/R94L/I118V/ | | | |
| T120S/T130A/K169E/T175A | | | |
| H18L/R29D/Y31L/Q33H/K36G/ | 1436 | | |
| M38I/T41A/M43R/M47T/ | | | |
| E81V/L85R/K89N/A91T/ | | | |
| F92P/K93V/R94L/I118V/ | | | |
| T120S/I127T/T130A/L142S/H188D | | | |
| C16S/H18L/R29D/Y31L/Q33H/ | 1437 | | |
| K36G/M38I/T41A/M43R/ | | | |
| M47T/E81V/L85R/K89N/ | | | |
| A91T/F92P/K93V/ | | | |
| R94L/T110A/I118V/H188D | | | |
| R29D/Y31L/Q33H/K36G/M38I/ | 1438 | | |
| T41A/M43R/M47T/A91G/ | | | |
| I118V/T120S/I127T/T130A/H188D | | | |
| R29D/Y31L/Q33H/K36G/M38I/ | 1439 | | |
| T41A/M43R/M47T/L70Q/ | | | |
| D76G/A91G/S103L/I118V/ | | | |
| T120S/I127T/T130A | | | |
| Y53C/L85R/K89N/A91T/F92P/ | 1440 | | |
| K93V/R94L/I118V/T120S/ | | | |
| I127T/T130A/K169E | | | |
| T62S/E81V/L85R/K89N/A91T/ | 1441 | | |
| F92P/K93V/R94L/I118V/ | | | |
| T120S/T130A/K169E | | | |
| Y53C/L70Q/D90G/T130A/ | 1442 | | |
| N149D/N152T/H188D | | | |
| H18L/R29D/Y31L/Q33H/K36G/ | 1443 | | |
| M38I/T41A/M43R/M47T/ | | | |
| E81V/L85R/K89N/A91T/F92P/K93V/R94L/ | | | |
| I118V/T120S/I127T/T130A/H188D | | | |
| H18L/R29D/Y31L/Q33H/K36G/ | 1444 | | |
| M38I/T41A/M43R/M47T/ | | | |
| E81V/L85R/K89N/A91T/F92P/ | | | |
| K93V/R94L/T130A/N149S | | | |
| I67T/L70Q/A91G/I118V/T120S/T130A | 1537 | 1538 | 1540 |
| S21P/L70Q/D90G/I118V/T120S/T130A | 1541 | 1542 | 1544 |

In some embodiments, the one or more amino acid modifications of a variant CD80 polypeptides provided herein produces at least one affinity-modified IgSF domain (e.g., IgV or IgC) or a specific binding fragment thereof relative to an IgSF domain contained in a wild-type or unmodified CD80 polypeptide such that the variant CD80 polypeptide exhibits altered (increased or decreased) binding activity or affinity for one or more binding partners, CTLA-4, PD-L1, or CD28, compared to a wild-type or unmodified CD80 polypeptide. The provided variant CD80 polypeptides containing at least one affinity-modified IgSF domain (e.g., IgV or IgC) or a specific binding fragment thereof exhibit altered (increased or decreased) binding activity or affinity for one or more cognate binding partners, CTLA-4, PD-L1, or CD28, compared to a wild-type or unmodified CD80 polypeptide. In some embodiments, a variant CD80 polypeptide has a binding affinity for CD28, PD-L1, or CTLA-4 that differs from that of a wild-type or unmodified CD80 polypeptide control sequence as determined by, for example, solid-phase ELISA immunoassays, flow cytometry or surface plasmon resonance (Biacore) assays. In some embodiments, the variant CD80 polypeptide has an increased binding affinity for CD28, PD-L1, and/or CTLA-4. In some embodiments, the variant CD80 polypeptide has an increased binding affinity for CTLA-4, and/or CD28. In some embodiments, the variant CD80 polypeptide has a decreased binding affinity for PD-L1, relative to a wild-type or unmodified CD80 polypeptide. The CD28, PD-L1 and/or the CTLA-4 can be a mammalian protein, such as a human protein or a murine protein.

Binding affinities for each of the binding partners are independent; that is, in some embodiments, a variant CD80 polypeptide has an altered binding affinity for one, two or three of CD28, PD-L1, and CTLA-4, relative to a wild-type or unmodified CD80 polypeptide. In some embodiments, a variant CD80 polypeptide has an increased binding affinity for one, two or three of CD28, PD-L1, and CTLA-4, relative to a wild-type or unmodified CD80 polypeptide. In some embodiments, a variant CD80 polypeptide has an increased binding affinity for one, two or three of CD28, PD-L1, and CTLA-4, and/or a decreased binding affinity for one, two or three of CD28, PD-L1, and CTLA-4, relative to a wild-type or unmodified CD80 polypeptide.

In some embodiments, the variant CD80 polypeptide has an increased binding affinity for CD28, relative to a wild-type or unmodified CD80 polypeptide. In some embodiments, the variant CD80 polypeptide has an increased binding affinity for PD-L1, relative to a wild-type or unmodified CD80 polypeptide. In some embodiments, the variant CD80 polypeptide has an increased binding affinity for CTLA-4, relative to a wild-type or unmodified CD80 polypeptide.

In some embodiments, the variant CD80 polypeptide has an increased binding affinity for PD-L1 and an increased binding affinity for CD28, relative to a wild-type or unmodified CD80 polypeptide. In some embodiments, the variant CD80 polypeptide has an increased binding affinity for CTLA-4 and an increased binding affinity for PD-L1, relative to a wild-type or unmodified CD80 polypeptide. In some embodiments, the variant CD80 polypeptide has an increased binding affinity for CD28 and an increased binding affinity for CTLA-4, relative to a wild-type or unmodified CD80 polypeptide. In some embodiments, the variant CD80 polypeptide has an increased binding affinity for CD28, PD-L1, and CTLA-4, relative to a wild-type or unmodified CD80 polypeptide.

In some embodiments, the variant CD80 polypeptide has a decreased binding affinity for PD-L1, relative to a wild-type or unmodified CD80 polypeptide.

In some embodiments, the variant CD80 polypeptide has an increased binding affinity for CTLA-4 and CD28, relative to a wild-type or unmodified CD80 polypeptide. In some embodiments, the variant CD80 polypeptide has a increased binding affinity for CTLA-4 and an decreased binding affinity for CD28, relative to a wild-type or unmodified CD80 polypeptide. In any of such embodiments, the variant CD80 polypeptide has a decreased binding affinity for PD-L1 and/or does not bind or substantially bind to PD-L1.

In some embodiments, a variant CD80 polypeptide with increased or greater binding affinity to CD28, PD-L1, and/or CTLA-4 will have an increase in binding affinity relative to the wild-type or unmodified CD80 polypeptide control of at least about 5%, such as at least about 10%, 15%, 20%, 25%, 35%, or 50% for the CD28, PD-L1, and/or CTLA-4 binding partner(s). In some embodiments, the increase in binding affinity relative to the wild-type or unmodified CD80 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 250-fold, 300-fold, 350-fold, 400-fold, or more. In such examples, the wild-type or unmodified CD80 polypeptide has the same sequence as the variant CD80 polypeptide except that it does not contain the one or more amino acid modifications (e.g., substitutions).

In some embodiments, a variant CD80 polypeptide with decreased or reduced binding affinity to a cognate binding partner(s) will have decrease in binding affinity relative to the wild-type or unmodified CD80 polypeptide control of at least 5%, such as at least about 10%, 15%, 20%, 30%, 40%, 50%, 60%, 70%, 80%, 90% or more for the binding partner(s). In some embodiments, the decrease in binding affinity relative to the wild-type or unmodified CD80 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold. In such examples, the wild-type or unmodified CD80 polypeptide has the same sequence as the variant CD80 polypeptide except that it does not contain the one or more amino acid modifications (e.g., substitutions).

In some embodiments, the equilibrium dissociation constant (K_{d}) of any of the foregoing embodiments to CD28, PD-L1, and/or CTLA-4 can be at least at or about 1×10⁻⁵ M, 1×10⁻⁶ M, 1×10⁻⁷ M, 1×10⁻⁸ M, 1×10⁻⁹ M, 1×10⁻¹⁰ M or 1×10⁻¹¹ M, or 1×10⁻¹² M or less.

Non-limiting examples of CD80 variant polypeptides with altered (e.g. increased or decreased) binding to binding partners are described in the examples, including those in which the mutations are contained in the full extracellular domain containing the IgV and IgC domain. Exemplary binding activities for binding cognate binding partners are shown in a flow-cytometry based assay based on mean fluorescence intensity (MFI) and comparison of binding to the corresponding unmodified or wild-type CD80 polypeptide. Among such variant polypeptides are polypeptides that exhibit an increase or decrease for a cognate binding partner, such as CD28, CTLA-4 and/or PD-L1 as described.

In some embodiments, the provided variant CD80 polypeptides containing at least one affinity-modified IgSF domain (e.g., IgV or IgC) or a specific binding fragment thereof relative to an IgSF domain contained in a wild-type or unmodified CD80 polypeptide exhibit altered (increases/stimulates or decreases/inhibits) signaling induced by one or more functional binding partner(s), such as CD28, PD-L1, and/or CTLA-4, expressed on the surface of a cell capable of signaling, such as a T-cell capable of releasing cytokine in response to intracellular signal, compared to a wild-type or unmodified CD80 polypeptide upon binding the one or more binding partner(s). In some embodiments, the altered signaling differs from that effected by a wild-type or unmodified CD80 polypeptide control sequence, e.g. in the same format (e.g. soluble), as determined by, for example, an assay that measures cytokine release (e.g., IL-2 release or IFN-gamma release), following incubation with the specified variant and/or wild-type or unmodified CD80 polypeptide. An exemplary assay is described in Examples 8-9. In exemplary assays, the cytokine release is a function of the sum of the signaling activities of the functional binding partners expressed on the surface of the cytokine-releasing cell.

Because CTLA-4 induces inhibitory signaling, increased CTLA-4 signaling results in a decrease in cytokine release in some exemplary assays. Conversely, decreased CTLA-4 signaling results in decreased inhibitory signaling, which does not decrease cytokine release and can result in increased cytokine release in some assays. Because CD28 signaling stimulates cytokine release, increased CD28 signaling results in increased cytokine release in exemplary assays. Conversely, decreased CD28 signaling results in decreased cytokine release in exemplary assays. Because PD-L1 induces inhibitory signaling when bound to PD-1, increased PD-L1 signaling results in a decrease in cytokine release in some exemplary assays. Conversely, decreased PD-L1 signaling results in decreased inhibitory signaling, which does not decrease cytokine release and can result in increased cytokine release in some assays.

In some embodiments, the variant CD80 polypeptide increases CD28-mediated signaling, relative to a wild-type or unmodified CD80 polypeptide. In some embodiments, the variant CD80 polypeptide decreases PD-L1, and/or CTLA-4-mediated signaling, relative to a wild-type or unmodified CD80 polypeptide. In some embodiments, the variant CD80 polypeptide increases CD28-mediated signaling and decreases PD-L1, and/or CTLA-4-mediated signaling, relative to a wild-type or unmodified CD80 polypeptide.

Binding affinities for each of the cognate binding partners are independent; thus, in some embodiments, a variant CD80 polypeptide can increase the signaling induced by one, two or three of CD28, PD-L1, and CTLA-4, and/or a decrease the signaling induced by one, two or three of CD28, PD-L1, and CTLA-4, relative to a wild-type or unmodified CD80 polypeptide.

In some embodiments, the variant CD80 polypeptide increases the signaling induced by CD28, upon binding, relative to a wild-type or unmodified CD80 polypeptide. In some embodiments, the variant CD80 decreases the signaling induced by PD-L1/PD-1, relative to a wild-type or unmodified CD80 polypeptide. In some embodiments, the variant CD80 polypeptide decreases the signaling induced by CTLA-4, relative to a wild-type or unmodified CD80 polypeptide.

In some embodiments, the variant CD80 polypeptide decreases the signaling induced by CTLA-4, and increases the signaling induced by CD28, relative to a wild-type or unmodified CD80 polypeptide. In some embodiments, the variant CD80 polypeptide decreases the signaling induced by PD-L1 and increases the signaling induced by CD28, relative to a wild-type or unmodified CD80 polypeptide.

In some embodiments, a variant CD80 polypeptide that stimulates or increases the signaling induced by CD28 will produce a signal that is at least 105%, 110%, 120%, 150%, 200%, 300%, 400%, or 500%, or more of the signal induced by the wild-type or unmodified CD80 polypeptide. In some embodiments, the increase in CD28-mediated signaling relative to the wild-type or unmodified CD80 polypeptide is more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 250-fold, 300-fold, 350-fold, 400-fold, or more. In such examples, the wild-type or unmodified CD80 polypeptide has the same sequence as the variant CD80 polypeptide except that it does not contain the one or more amino acid modifications (e.g., substitutions).

In some embodiments, a variant CD80 polypeptide that inhibits or decreases the inhibitory signaling induced by CTLA-4 or PD-1/PD-L1 will produce a signal that is 90%, 85%, 80%, 75%, 70%, 65%, 60%, 55%, 50%, 45%, 40%, 35%, 30%, 25%, 20%, 15%, 10%, 5%, or less, of the signal induced by the wild-type or unmodified CD80 polypeptide. In such examples, the wild-type or unmodified CD80 polypeptide has the same sequence as the variant CD80 polypeptide except that it does not contain the one or more amino acid modifications (e.g., substitutions).

In some embodiments, a variant CD80 polypeptide that affects the inhibitory signaling induced by CTLA-4 and/or PD-L1, and/or affects the signaling by CD28 will yield a sum of the PD-L1, CTLA-4 and CD28 signaling that is greater than the sum of the PD-L1, CTLA-4 and CD28 signaling effected by the corresponding wild-type or unmodified CD80 polypeptide. In such embodiments, the sum of the PD-L1, CTLA-4 and CD28 signaling is at least 105%, 110%, 120%, 150%, 200%, 300%, 400%, or 500%, or more of the signal effected by the corresponding wild-type or unmodified CD80 polypeptide. In such examples, the corresponding wild-type or unmodified CD80 polypeptide has the same sequence as the variant CD80 polypeptide except that it does not contain the one or more amino acid modifications (e.g., substitutions).

Non-limiting examples of CD80 variant polypeptides with altered (e.g. increased or decreased) signaling induced following interactions with one or more functional binding partners, e.g. CD28, PD-L1, and/or CTLA-4, are described in the examples. Among provided CD80 variant polypeptides include those in which the mutations are contained in the full extracellular domain containing the IgV and IgC domain. Exemplary functional activities are shown in a reporter-based assay based on changed in fluorescence of a reporter in a T cell reporter Jurkat cell line, including in comparison to the corresponding unmodified or wild-type CD80 polypeptide. Among such variant polypeptides are polypeptides that exhibit an increase in CD28 costimulation or agonism as described. 1. CD28

In some embodiments, the variant CD80 polypeptide exhibits increased affinity for the ectodomain of CD28 compared to a wild-type or unmodified CD80 polypeptide. In some embodiments, the variant CD80 polypeptide exhibits increased affinity to the ectodomain of CD28 compared to a wildtype or unmodified CD80 polypeptide, such as comprising the sequence set forth in SEQ ID NO: 2, 76, 150, or 1245. In some embodiments, the increased affinity to the ectodomain of CD28 is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold 60-fold, 70-fold, 80-fold, 90-fold, 100-fold, 150-fold, or 200-fold, compared to binding affinity of the unmodified CD80 for the ectodomain of CD28.

In some embodiments, the variant CD80 polypeptide exhibits increased affinity for the ectodomain of CD28 and the ectodomain of CTLA-4 compared to a wildtype or unmodified CD80 polypeptide, such as comprising the sequence set forth in SEQ ID NO: 2, 76, 150, or 1245. In some embodiments, the variant CD80 polypeptide exhibits increased affinity for the ectodomain of CD28 and the ectodomain of PD-L1 compared to a wildtype or unmodified CD80 polypeptide, such as comprising the sequence set forth in SEQ ID NO: 2, 76, 150, or 1245. In some embodiments, the variant CD80 polypeptide exhibits increased affinity for the ectodomain of CD28, the ectodomain of PD-L1 and the ectodomain of CTLA-4 compared to wild-type or an unmodified CD80 polypeptide, such as comprising the sequence set forth in SEQ ID NO: 2, 76, 150, or 1245. In some embodiments, the increased affinity to the ectodomain of CD28 and one or both of CTLA-4 and PD-L1 is independently increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, or 450-fold compared to binding affinity of the unmodified CD80 for the ectodomain of CTLA-4 or PD-L1.

In some embodiments, the variant CD80 polypeptide exhibits increased affinity for the ectodomain of CD28 and the ectodomain of CTLA-4, compared to wild-type or unmodified CD80 polypeptide, such as comprising the sequence set forth in SEQ ID NO: 2, 76, 150, or 1245. In some embodiments, the variant CD80 polypeptide exhibits increased affinity for the ectodomain of CD28 and the ectodomain of PD-L1, compared to wild-type or unmodified CD80 polypeptide, such as comprising the sequence set forth in SEQ ID NO: 2, 76, 150, or 1245. In some embodiments, the variant CD80 polypeptide exhibits increased affinity for the ectodomain of CD28, the ectodomain of CTLA-4, and the ectodomain of PD-L1, compared to wild-type or unmodified CD80 polypeptide, such as comprising the sequence set forth in SEQ ID NO: 2, 76, 150, or 1245. In some embodiments, the increased affinity to the ectodomain of CD28 is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold or 60-fold compared to binding affinity of the unmodified CD80 for the ectodomain of CD28.

Non-limiting examples of CD80 variant polypeptides with altered (e.g. increased binding to CD28 are described in the examples. Exemplary binding activities for binding CD28 are shown in a flow-cytometry based assay based on mean fluorescence intensity (MFI) and comparison of binding to the corresponding unmodified or wild-type CD80 polypeptide. Among such variant polypeptides are polypeptides that exhibit an increase binding for CD28, e.g. human CD28, as described. Further, non-limiting examples of CD80 variant polypeptides with altered (e.g. increased) signaling induced following interactions with one or more functional binding partners, e.g. CD28, are described in the examples. Exemplary functional activities are shown, in some aspects, in an mixed lymphocyte reaction and/or reporter-based assay based on changed in fluorescence of a reporter in a T cell reporter Jurkat cell line, including in comparison to the corresponding unmodified or wild-type CD80 polypeptide. Among such variant polypeptides are polypeptides that exhibit an increase in CD28 costimulation or agonism as described.

Among non-limiting examples of such variant polypeptide include, in some of these embodiments, the variant CD80 polypeptide that exhibits increased binding affinity for CD28 compared to a wild-type or unmodified CD80 polypeptide has one or more amino acid modifications (e.g., substitutions) corresponding to positions 12, 13, 18, 20, 22, 23, 24, 26, 27, 31, 35, 41, 42, 43, 46, 47, 54, 55, 57, 58, 61, 62, 67, 68, 69, 70, 71, 72, 79, 83, 84, 85, 88, 90, 93, 94, and/or 95 of SEQ ID NO: 2, 76, 150, or 1245. In some of these embodiments, the variant CD80 polypeptide that exhibits increased binding affinity for CD28 compared to a wild-type or unmodified CD80 polypeptide has one or more amino acid modifications (e.g., substitutions) corresponding to positions 23, 26, 35, 46, 55, 57, 58, 71, 79, and/or 84 of SEQ ID NO: 2, 76, 150, or 1245.

In some embodiments, the variant CD80 polypeptide has one or more amino acid substitutions selected from the group consisting of A12T, T13R, S15T, H18A, H18C, H18F, H18I, H18T, H18V, H18Y, V20I, S21P, V22A, V22D, V22L, E23D, E23G, E24D, A26D, A26E, A26G, A26H, A26K, A26N, A26P, A26Q, A26R, A26S, A26T, Q27H, Q27L, Q27R, Y31H, Q33R, E35D, E35G, K37E, M38I, T41S, M42V, M43I, M43L, D46E, D46N, D46V, M47I, M47L, M47V, M47Y, N48K, N48Y, Y53F, K54E, N55I, T57A, T57I, I58V, I61F, I61V, T62A, T62N, T62S, N64S, I67L, V68E, V68I, V68L, V68M, I69F, L70M, L70Q, L70R, A71D, A71G, L72P, L72V, 179I, T79M, V83I, V84I, L85M, L85Q, Y87C, Y87D, Y87N, E88D, E88V, D90G, D90N, D90P, A91G, A91S, K93E, K93R, R94L, R94Q, R94W, E95K, E95V, and L97Q. In some embodiments, the variant CD80 polypeptide has one or more amino acid substitutions selected from the group consisting of T13R, S15T, H18A, H18C, H18F, H18I, H18T, H18V, V20I, V22D, V22L, E23D, E23G, E24D, A26D, A26E, A26G, A26H, A26K, A26N, A26P, A26Q, A26R, A26S, A26T, Q27H, Q27L, Q33R, E35D, E35G, T41S, M42V, M43L, D46E, D46N, D46V, M47I, M47L, M47V, M47Y, N48K, N48Y, Y53F, K54E, N55I, T57A, T57I, I58V, I61F, I61V, T62A, T62N, I67L, V68E, V68I, V68L, I69F, L70M, A71D, A71G, L72V, 179I, T79M, V84I, L85M, L85Q, Y87C, Y87D, E88V, D90P, R94Q, R94W, E95V, L97Q.

In some embodiments, the one or more amino acid substitution is Q27H/T41S/A71D, V20I/M47V/T57I/V84I, V20I/M47V/A71D, A71D/L72V/E95K, V22L/E35G/A71D/L72P, E35D/A71D, E35D/I67L/A71D, Q27H/E35G/A71D/L72P/T79I, T13R/M42V/M47/A71D, E35D, E35D/M47I/L70M, E35D/A71D/L72V, E35D/M43L/L70M, A26P/E35D/M43I/L85Q/E88D, E35D/D46V/L85Q, Q27L/E35D/M47/T57I/L70Q/E88D, M47V/I69F/A71D/V83I, E35D/T57A/A71D/L85Q, H18Y/A26T/E35D/A71D/L85Q, E35D/M47L, E23D/M42V/M43I/I58V/L70R, V68M/L70M/A71D/E95K, N55I/T57I/I69F, E35D/M43I/A71D, T41S/T57I/L70R, H18Y/A71D/L72P/E88V, V20I/A71D, E23G/A26S/E35D/T62N/A71D/L72V/L85M, A12T/E24D/E35D/D46V/I61V/L72P/E95V, E35G/K54E/A71D/L72P, L70Q/A71D, A26E/E35D/M47L/L85Q, D46E/A71D, Y31H/E35D/T41S/V68L/K93R/R94W, V22A/E35D/V68E/A71D, E35D/D46E/M47V/V68M/D90G/K93E, E35D/N48K/L72V, D46V/M47I/A71G, M47I/A71G, E35D/M43I/M47L/L85M, E35D/M43I/D46E/A71G/L85M, H18Y/E35D/M47L/A71G/A91S, E35D/M47I/N48K/I61F, E35D/M47V/T62S/L85Q, M43I/M47L/A71G, E35D/M47V, E35D/M47L/A71G/L85M, V22A/E35D/M47L/A71G, E35D/M47L/A71G, E35D/D46E/M47I, Q27H/E35D/M47I, E35D/D46E/L85M, E35D/D46E/A91G, E35D/D46E, H18Y/E35D, Q27L/E35D/M47V/I61V/L85M, E35D/M47V/I61V/L85M, E35D/M47V/N48K/L85M, H18Y/E35D/M47V/N48K, A26E/Q27R/E35D/M47L/N48Y/L85Q, E35D/M47I/T62S/L85Q/E88D, E24D/Q27R/E35D/T41S/M47V/L85Q, S15T/H18Y/E35D/M47V/T62A/N64S/A71G/L85Q/D90N, E35D/M47L/V68M/A71G/L85Q/D90G, H18Y/E35D/M47I/V68M/A71G/R94L, H18Y/Y22A/E35D/T41S/M47V/T62N/A71G/A91G, E35D/D46E/M47I/T62A/V68M/L85M/Y87C, E35D/D46E/M47I/V68M/L85M, E35D/D46E/M47L/V68M/A71G/Y87C/K93R, E35D/D46E/M47L/V68M/T79M/L85M, E35D/D46E/M47V/V68M/L85Q, E35D/M43I/M47L/V68M, E35D/M47I/V68M/Y87N, E35D/M47L/Y53F/V68M/A71G/K93R/E95V, E35D/M47V/N48K/V68M/A71G/L85M, E35D/M47V/N48K/V68M/L85M, E35D/M47V/V68M/L85M, E35D/M47V/V68M/L85M/Y87D, E35D/T41S/D46E/M47I/V68M/K93R/E95V, H18Y/E35D/D46E/M47I/V68M/R94L, H18Y/E35D/D46E/M47I/V68M/R94L, H18Y/E35D/M47I/V68M/Y87N, H18Y/E35D/M47I/V68M/Y87N, H18Y/E35D/M47L/V68M/A71G/L85M, H18Y/E35D/M47L/V68M/A71G/L85M, H18Y/E35D/M47L/V68M/E95V/L97Q, H18Y/E35D/M47L/Y53F/V68M/A71G, H18Y/E35D/M47L/Y53F/V68M/A71G/K93R/E95V, H18Y/E35D/M47L/Y53F/V68M/A71G/K93R/E95V, H18Y/E35D/M47V/V68M/L85M, H18Y/E35D/M47V/V68M/L85M, H18Y/E35D/V68M/A71G/R94Q/E95V, H18Y/E35D/V68M/L85M/R94Q, H18Y/E35D/V68M/T79M/L85M, H18Y/V22D/E35D/M47V/N48K/V68M, S21P/E35D/K37E/D46E/M47I/V68M, S21P/E35D/K37E/D46E/M47I/V68M/R94L, T13R/Q33R/E35D/M38I/M47L/V68M/E95V/L97Q, T13R/Q33R/E35D/M47L/V68M/L85M, V22D/E24D/E35D/M47L/V68M, V22D/E24D/E35D/M47L/V68M/L85M/D90G, V22D/E24D/E35D/M47V/V68M, H18Y/E35D/M47V/V68M/A71G, H18C/A26P/E35D/M47L/V68M/A71G, H18I/A26P/E35D/M47V/V68M/A71G, H18L/A26N/D46E/V68M/A71G/D90G, H18L/E35D/M47V/V68M/A71G/D90G, H18T/A26N/E35D/M47L/V68M/A71G, H18V/A26K/E35D/M47L/V68M/A71G, H18V/A26N/E35D/M47V/V68M/A71G, H18V/A26P/E35D/M47V/V68L/A71G, H18V/A26P/E35D/M47L/V68M/A71G, H18V/E35D/M47V/V68M/A71G/D90G, H18Y/A26P/E35D/M47U/V68M/A71G, H18Y/A26P/E35D/M47V/V68M/A71G, H18Y/E35D/M47V/V68L/A71G/D90G, H18Y/E35D/M47V/V68M/A71G/D90G, A26P/E35D/M47I/V68M/A71G/D90G, H18V/A26G/E35D/M47V/V68M/A71G/D90G, H18V/A26S/E35D/M47L/V68M/A71G/D90G, H18V/A26R/E35D/M47L/V68M/A71G/D90G, H18V/A26D/E35D/M47V/V68M/A71G/D90G, H18V/A26Q/E35D/M47V/V68L/A71G/D90G, H18A/A26P/E35D/M47L/V68M/A71G/D90G, H18A/A26N/E35D/M47L/V68M/A71G/D90G, H18F/A26P/E35D/M47I/V68M/A71G/D90G, H18F/A26H/E35D/M47L/V68M/A71G/D90G, H18F/A26N/E35D/M47V/V68M/A71G/D90K, H18Y/A26P/E35D/M47V/V68U/A71G/D90G, H18Y/A26Q/E35D/M47T/V68M/A71G/D90G, H18R/A26P/E35D/D46N/M47V/V68M/A71G/D90P, or H18F/A26D/E35D/D46E/M47T/V68M/A71G/D90G.

2. PD-L1

In some embodiments, the variant CD80 polypeptide exhibits increased affinity to PD-L1 compared to the wild-type or unmodified CD80 polypeptide. In some embodiments, the variant CD80 polypeptide exhibits increased affinity for the ectodomain of PD-L1 compared to wild-type or an unmodified CD80 polypeptide, such as comprising the sequence set forth in SEQ ID NO: 2, 76, 150, or 1245. In some embodiments, the increased affinity to the ectodomain of PD-L1 is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, or 450-fold compared to binding affinity of the unmodified CD80 for the ectodomain of PD-L1.

In some embodiments, the variant CD80 polypeptide exhibits increased affinity for the ectodomain of PD-L1, and increased affinity for the ectodomain of CTLA-4, compared to wild-type or unmodified CD80 polypeptide, such as comprising the sequence set forth in SEQ ID NO: 2, 76, 150, or 1245. In some embodiments, the variant CD80 polypeptide exhibits increased affinity for the ectodomain of PD-L1, and increased affinity for the ectodomain of CD28, compared to wild-type or unmodified CD80 polypeptide, such as comprising the sequence set forth in SEQ ID NO: 2, 76, 150, or 1245. In some embodiments, the variant CD80 polypeptide exhibits increased affinity for the ectodomain of PD-L1, and increased affinity for the ectodomain of CD28, and increased affinity for the ectodomain of CTLA-4, compared to wild-type or unmodified CD80 polypeptide, such as comprising the sequence set forth in SEQ ID NO: 2, 76, 150, or 1245. In some embodiments, the increased affinity to the ectodomain of PD-L1 is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold or 60-fold compared to binding affinity of the unmodified CD80 for the ectodomain of PD-L1.

Non-limiting examples of CD80 variant polypeptides with altered (e.g. increased) binding to PD-L1 are described in the examples. Exemplary binding activities for binding PD-L1 are shown in a flow-cytometry based assay based on mean fluorescence intensity (MFI) and comparison of binding to the corresponding unmodified or wild-type CD80 polypeptide. Among such variant polypeptides are polypeptides that exhibit an increase binding for PD-L1, e.g. human PD-L1, as described. Further, non-limiting examples of CD80 variant polypeptides with altered (e.g. increased) signaling induced following interactions with one or more functional binding partners, e.g. PD-L1, are described in the examples. Exemplary functional activities are shown, in some aspects, in an mixed lymphocyte reaction and/or reporter-based assay based on changed in fluorescence of a reporter in a T cell reporter Jurkat cell line, including in comparison to the corresponding unmodified or wild-type CD80 polypeptide. Among such variant polypeptides are polypeptides that exhibit an increase in PD-L1-dependent CD28 costimulation or agonism as described.

Among non-limiting examples of such variant polypeptide include, in some of these embodiments, the variant CD80 polypeptide that exhibits increased binding affinity for PD-L1 compared to a wild-type or unmodified CD80 polypeptide has one or more amino acid modifications (e.g., substitutions) corresponding to positions 7, 12, 13, 15, 16, 18, 20, 22, 23, 24, 25, 26, 27, 28, 29, 30, 31, 33, 34, 35, 36, 37, 38, 41, 42, 43, 44, 46, 47, 48, 51, 53, 54, 55, 57, 58, 61, 62, 63, 65, 67, 68, 69, 70, 71, 72, 73, 74, 76, 77, 78, 79, 81, 82, 83, 84, 85, 86, 87, 88, 89, 90, 91, 92, 93, 94, 95, and/or 97 of SEQ ID NO: 2, 76, 150, or 1245. In some of these embodiments, the variant CD80 polypeptide that exhibits increased binding affinity for PD-L1 compared to a wild-type or unmodified CD80 polypeptide has one or more amino acid modifications (e.g., substitutions) corresponding to positions 7, 23, 26, 30, 34, 35, 46, 51, 55, 57, 58, 65, 71, 73, 78, 79, 82, and/or 84, of SEQ ID NO: 2, 76, 150, or 1245.

In some embodiments, the variant CD80 polypeptide has one or more amino acid substitutions selected from the group consisting of E7D, A12V, T13A, T13R, S15P, S15T, C16R, H18A, H18C, H18F, H18I, H18T, H18V, H18L, H18Y, V20A, V20I, S21P, V22A, V22D, V22I, V22L, E23D, E23G, E24D, L25S, A26D, A26E, A26G, A26H, A26K, A26N, A26P, A26Q, A26R, A26S, A26T, Q27H, Q27L, Q27R, R29C, T28Y, R29H, I30T, I30V, Y31H, Y31S, Q33E, Q33H, Q33K, Q33L, Q33R, K34E, E35D, K36R, K37E, M38I, M38T, M38V, T41A, T41S, M42I, M42V, M43I, M43L, M43T, M43V, S44P, D46E, D46N, D46V, M47F, M47I, M47L, M47T, M47V, N48D, N48H, N48K, N48R, N48S, N48T, N48Y, P51A, Y53F, Y53H, K54R, N55D, N55I, T57I, I58V, I61F, I61N, I61V, T62A, T62N, T62S, N63D, N64S, L65P, I67L, I67T, V68A, V68I, V68L, V68M, I69F, L70M, L70P, L70Q, L70R, A71D, A71G, L72P, L72V, R73S, P74S, D76H, E77A, G78A, T79A, T79I, T79L, T79M, T79P, E81G, E81K, C82R, V83A, V83I, V84A, V84I, L85E, L85M, L85Q, K86E, K86M, Y87C, Y87D, Y87H, Y87N, Y87Q, E88D, E88G, K89E, K89N, D90G, D90N, D90P, A91G, A91S, A91T, A91V, F92L, F92S, F92V, F92Y, K93E, K93R, K93T, R94L, R94Q, R94W, E95D, E95K, E95V, L97M, L97Q, and L97R. In some embodiments, the variant CD80 polypeptide has one or more amino acid substitutions selected from the group consisting of E7D, T13A, T13R, S15T, C16R, H18A, H18C, H18F, H18I, H18T, H18V, V20A, V20I, V22D, V22I, V22L, E23D, E23G, E24D, L25S, A26D, A26E, A26G, A26H, A26K, A26N, A26P, A26Q, A26R, A26S, A26T, Q27H, Q27L, 130T, I30V, Q33E, Q33K, Q33L, Q33R, K34E, E35D, K36R, T41S, M42I, M42V, M43L, M43T, D46E, D46N, D46V, M47F, M47I, M47L, M47V, N48D, N48H, N48K, N48R, N48S, N48T, N48Y, P51A, Y53F, K54R, N55D, N55I, T57I, I58V, I61F, I61V, T62A, T62N, L65P, I67L, V68I, V68L, I69F, L70M, A71D, A71G, L72V, R73S, P74S, D76H, G78A, T79A, T79I, T79L, T79M, T79P, E81G, E81K, C82R, V84A, V84I, L85E, L85M, L85Q, K86M, Y87C, Y87D, D90P, F92S, F92V, R94Q, R94W, E95D, E95V, L97M, and L97Q.

In some embodiments, the one or more amino acid substitution is Q27H/T41S/A71D, I30T/L70R, T13R/C16R/L70Q/A71D, T57I, M43I/C82R, V22L/M38V/M47T/A71D/L85M, I30V/T57I/L70P/A71D/A91T, V22I/L70M/A71D, N55D/K86M, L72P/T79I, L70P/F92S, T79P, E35D/M47I/L65P/D90N, L25S/E35D/M47I/D90N, S44P/167T/P74S/E81G/E95D, A71D, T13A/I61N/A71D, E81K, A12V/M47V/L70M, K34E/T41A/L72V, T41S/A71D/V84A, E35D/A71D, E35D/M47I, K36R/G78A, Q33E/T41A, M47V/N48H, M47L/V68A, S44P/A71D, Q27H/M43I/A71D/R73S, E35D/T57I/L70Q/A71D, M47I/E88D, M42I/I61V/A71D, P51A/A71D, H18Y/M47I/T57I/A71G, V20I/M47V/T57I/V84I, V20I/M47V/A71D, A71D/L72V/E95K, E35D/A71D, E35D/I67L/A71D, T13R/M42V/M47I/A71D, E35D, E35D/M47I/L70M, E35D/A71D/L72V, E35D/M43L/L70M, A26P/E35D/M43I/L85Q/E88D, E35D/D46V/L85Q, M47V/I69F/A71D/V83I, H18Y/A26T/E35D/A71D/L85Q, E35D/M47L, E23D/M42V/M43I/I58V/L70R, V68M/L70M/A71D/E95K, N55I/T57I/I69F, E35D/M43I/A71D, T41S/T57I/L70R, V20I/A71D, E23G/A26S/E35D/T62N/A71D/L72V/L85M, V22L/E35D/M43L/A71G/D76H, A26E/E35D/M47L/L85Q, D46E/A71D, Y31H/E35D/T41S/V68L/K93R/R94W, A26E/Q33R/E35D/M47L/L85Q/K86E, A26E/Q33R/E35D/M47L/L85Q, E35D/M47L/L85Q, A26E/Q33L/E35D/M47L/L85Q, A26E/Q33L/E35D/M47L, H18Y/A26E/Q33L/E35D/M47L/L85Q, Q33L/E35D/M47I, H18Y/Q33L/E35D/M47I, Q33L/E35D/D46E/M47I, Q33R/E35D/D46E/M47I, H18Y/E35D/M47L, Q33L/E35D/M47V, Q33L/E35D/M47V/T79A, Q33L/E35D/T41S/M47V, Q33L/E35D/M47I/L85Q, Q33L/E35D/M47I/T62N/L85Q, Q33L/E35D/M47V/L85Q, A26E/E35D/M43T/M47L/L85Q/R94Q, Q33R/E35D/K37E/M47V/L85Q, V22A/E23D/Q33L/E35D/M47V, E24D/Q33L/E35D/M47V/K54R/L85Q, S15P/Q33L/E35D/M47L/L85Q, E7D/E35D/M47I/L97Q, Q33L/E35D/T41S/M43I, E35D/M47I/K54R/L85E, Q33K/E35D/D46V/L85Q, Y31S/E35D/M47L/T79L/E88G, H18L/V22A/E35D/M47L/N48T/L85Q, Q27H/E35D/M47L/L85Q/R94Q/E95K, Q33K/E35D/M47V/K89E/K93R, E35D/M47I/E77A/L85Q/R94W, A26E/E35D/M43I/M47L/L85Q/K86E/R94W, Q27H/Q33L/E35D/M47V/N55D/L85Q/K89N, H18Y/V20A/Q33L/E35D/M47V/V53F, Q33L/E35D/M47L/A71G/F92S, V22A/R29H/E35D/D46E/M47I, Q33L/E35D/M43I/L85Q/R94W, H18Y/E35D/V68M/L97Q, Q33L/E35D/M47L/V68M/L85Q/E88D, Q33L/E35D/M43V/M47I/A71G, E35D/M47L/A71G/L97Q, E35D/M47V/A71G/L85M/L97Q, H18Y/Y31H/E35D/M47V/A71G/L85Q, E35D/D46E/M47V/L97Q, E35D/D46V/M47I/A71G/F92V, E35D/M47V/T62A/A71G/V83A/Y87H/L97M, Q33L/E35D/N48K/L85Q/L97Q, E35D/L85Q/K93T/E95V/L97Q, E35D/M47V/N48K/V68M/K89N, Q33L/E35D/M47I/N48D/A71G, Q27H/E35D/M47I/L85Q/D90G, E35D/M47I/L85Q/D90G, E35D/M47I/T62S/L85Q, A26E/E35D/M47L/A71G, E35D/M47I/Y87Q/K89E, V22A/E35D/M47I/Y87N, H18Y/A26E/E35D/M47L/L85Q/D90G, E35D/M47L/A71G/L85Q, E35D/M47V/A71G/E88D, E35D/A71G, E35D/M47V/A71G, I30V/E35D/M47V/A71G/A91V, V22D/E35D/M47L/L85Q, H18Y/E35D/N48K, E35D/T41S/M47V/A71G/K89N, E35D/M47V/N48T/L85Q, E35D/D46E/M47V/A71D/D90G, E35D/T41S/M43I/A71G/D90G, E35D/T41S/M43I/M47V/A71G, E35D/T41S/M43I/M47L/A71G, H18Y/Y22A/E35D/M47V/T62S/A71G, H18Y/A26E/E35D/M47L/V68M/A71G/D90G, E35D/K37E/M47V/N48D/L85Q/D90N, Q27H/E35D/D46V/M47L/A71G, V22L/Q27H/E35D/M47I/A71G, E35D/D46V/M47L/V68M/L85Q/E88D, E35D/T41S/M43V/M47I/L70M/A71G, E35D/D46E/M47V/N63D/L85Q, E35D/D46E/M47V/V68M/D90G/K93E, E35D/M43I/M47V/K89N, E35D/M47L/A71G/L85M/F92Y, V22D/E35D/M47L/L70M/L97Q, E35D/T41S/M47V/L97Q, E35D/Y53H/A71G/D90G/L97R, Q33L/E35D/M43U/Y53F/T62S/L85Q, E35D/M38T/D46E/M47V/N48S, Q33R/E35D/M47V/N48K/L85M/F92L, E35D/M38T/M43V/M47V/N48R/L85Q, T28Y/Q33H/E35D/D46V/M47U/A71G, E35D/N48K/L72V, E35D/T41S/N48T, D46V/M47I/A71G, M47I/A71G, E35D/M43I/M47L/L85M, E35D/M43I/D46E/A71G/L85M, H18Y/E35D/M47L/A71G/A91S, E35D/M47I/N48K/I61F, E35D/M47V/T62S/L85Q, M43I/M47L/A71G, E35D/M47V, E35D/M47L/A71G/L85M, V22A/E35D/M47L/A71G, E35D/M47L/A71G, E35D/D46E/M47I, Q27H/E35D/M47I, E35D/D46E/L85M, E35D/D46E/A91G, E35D/D46E, E35D/L97R, H18Y/E35D, Q27L/E35D/M47V/I61V/L85M, E35D/M47V/I61V/L85M, E35D/M47V/L85M/R94Q, E35D/M47V/N48K/L85M, H18Y/E35D/M47V/N48K, A26E/Q27R/E35D/M47L/N48Y/L85Q, E35D/D46E/M47L/V68M/L85Q/F92L, E35D/M47I/T62S/L85Q/E88D, E24D/Q27R/E35D/T41S/M47V/L85Q, S15T/H18Y/E35D/M47V/T62A/N64S/A71G/L85Q/D90N, E35D/M47L/V68M/A71G/L85Q/D90G, H18Y/E35D/M47I/V68M/A71G/R94L, Q33R/M47V/T62N/A71G, H18Y/Y22A/E35D/T41S/M47V/T62N/A71G/A91G, E24D/E35D/M47L/V68M/E95V/L97Q, E35D/D46E/M47I/T62A/V68M/L85M/Y87C, E35D/D46E/M47I/V68M/L85M, E35D/D46E/M47L/V68M/A71G/Y87C/K93R, E35D/D46E/M47L/V68M/T79M/L85M, E35D/D46E/M47L/V68M/T79M/L85M/L97Q, E35D/D46E/M47V/V68M/L85Q, E35D/M43I/M47L/V68M, E35D/M47I/V68M/Y87N, E35D/M47L/V68M/E95V/L97Q, E35D/M47L/Y53F/V68M/A71G/K93R/E95V, E35D/M47V/N48K/V68M/A71G/L85M, E35D/M47V/N48K/V68M/L85M, E35D/M47V/V68M/L85M, E35D/M47V/V68M/L85M/Y87D, E35D/T41S/D46E/M47I/V68M/K93R/E95V, H18Y/E35D/D46E/M47I/V68M/R94L, H18Y/E35D/D46E/M47I/V68M/R94L, H18Y/E35D/M38I/M47L/V68M/L85M, H18Y/E35D/M47I/V68M/Y87N, H18Y/E35D/M47I/V68M/Y87N, H18Y/E35D/M47L/V68M/A71G/L85M, H18Y/E35D/M47L/V68M/A71G/L85M, H18Y/E35D/M47L/V68M/E95V/L97Q, H18Y/E35D/M47L/V68M/E95V/L97Q, H18Y/E35D/M47L/Y53F/V68M/A71G, H18Y/E35D/M47L/Y53F/V68M/A71G, H18Y/E35D/M47L/Y53F/V68M/A71G/K93R/E95V, H18Y/E35D/M47L/Y53F/V68M/A71G/K93R/E95V, H18Y/E35D/M47V/V68M/L85M, H18Y/E35D/M47V/V68M/L85M, H18Y/E35D/V68M/A71G/R94Q/E95V, H18Y/E35D/V68M/A71G/R94Q/E95V, H18Y/E35D/V68M/L85M/R94Q, H18Y/E35D/V68M/L85M/R94Q, H18Y/E35D/V68M/T79M/L85M, H18Y/V22D/E35D/M47V/N48K/V68M, Q27L/Q33L/E35D/T41S/M47V/N48K/V68M/L85M, Q33L/E35D/M47V/T62S/V68M/L85M, Q33R/E35D/M38I/M47L/V68M, R29C/E35D/M47L/V68M/A71G/L85M, S21P/E35D/K37E/D46E/M47I/V68M, S21P/E35D/K37E/D46E/M47I/V68M/R94L, T13R/E35D/M47L/V68M, T13R/Q27L/Q33L/E35D/T41S/M47V/N48K/V68M/L85M, T13R/Q33L/E35D/M47L/V68M/L85M, T13R/Q33L/E35D/M47V/T62S/V68M/L85M, T13R/Q33R/E35D/M38I/M47L/V68M, T13R/Q33R/E35D/M38I/M47L/V68M/E95V/L97Q, T13R/Q33R/E35D/M38I/M47L/V68M/L85M, T13R/Q33R/E35D/M38I/M47L/V68M/L85M/R94Q, T13R/Q33R/E35D/M47L/V68M, T13R/Q33R/E35D/M47L/V68M/L85M, V22D/E24D/E35D/M47L/V68M, V22D/E24D/E35D/M47L/V68M/L85M/D90G, V22D/E24D/E35D/M47V/V68M, H18Y/E35D/M47V/V68M/A71G, H18C/A26P/E35D/M47L/V68M/A71G, H18I/A26P/E35D/M47V/V68M/A71G, H18L/A26N/D46E/V68M/A71G/D90G, H18L/E35D/M47V/V68M/A71G/D90G, H18T/A26N/E35D/M47L/V68M/A71G, H18V/A26K/E35D/M47L/V68M/A71G, H18V/A26N/E35D/M47V/V68M/A71G, H18V/A26P/E35D/M47V/V68L/A71G, H18V/A26P/E35D/M47L/V68M/A71G, H18V/E35D/M47V/V68M/A71G/D90G, H18Y/A26P/E35D/M47I/V68M/A71G, H18Y/A26P/E35D/M47V/V68M/A71G, H18Y/E35D/M47V/V68L/A71G/D90G, H18Y/E35D/M47V/V68M/A71G/D90G, A26P/E35D/M47I/V68M/A71G/D90G, H18V/A26G/E35D/M47V/V68M/A71G/D90G, H18V/A26S/E35D/M47L/V68M/A71G/D90G, H18V/A26R/E35D/M47L/V68M/A71G/D90G, H18V/A26D/E35D/M47V/V68M/A71G/D90G, H18V/A26Q/E35D/M47V/V68L/A71G/D90G, H18A/A26P/E35D/M47L/V68M/A71G/D90G, H18A/A26N/E35D/M47L/V68M/A71G/D90G, H18F/A26P/E35D/M47I/V68M/A71G/D90G, H18F/A26H/E35D/M47L/V68M/A71G/D90G, H18F/A26N/E35D/M47V/V68M/A71G/D90K, H18Y/A26N/E35D/M47F/V68M/A71G/D90G, H18Y/A26P/E35D/M47V/V68U/A71G/D90G, H18Y/A26Q/E35D/M47T/V68M/A71G/D90G, H18R/A26P/E35D/D46N/M47V/V68M/A71G/D90P, or H18F/A26D/E35D/D46E/M47T/V68M/A71G/D90G.

In some embodiments, the variant CD80 polypeptides provided herein, that exhibit increased affinity for the ectodomain of PD-L1, compared to a wild-type or unmodified CD80 polypeptide, results in decreased inhibitory signal from the binding of PD-L1 an PD-1. In some embodiments, a variant CD80 polypeptide that inhibits or decreases the inhibitory signaling induced by PD-L1 and PD-1 will produce a signal that is 90%, 85%, 80%, 75%, 70%, 65%, 60%, 55%, 50%, 45%, 40%, 35%, 30%, 25%, 20%, 15%, 10%, 5%, or less, of the PD-L1/PD-1 signal in the presence of the wild-type or unmodified CD80 polypeptide. In such examples, the wild-type or unmodified CD80 polypeptide has the same sequence as the variant CD80 polypeptide except that it does not contain the one or more amino acid modifications (e.g., substitutions).

In some embodiments, the variant CD80 polypeptides provided herein, that exhibit increased affinity for the ectodomain of PD-L1, compared to a wild-type or unmodified CD80 polypeptide, can exhibit PD-L1-dependent CD28 costimulation or can effect PD-L1-dependent CD28 costimulatory activity. In some embodiments, wherein a variant CD80 polypeptide mediates or effects PD-L1-dependent CD28 costimulatory activity, the affinity of the variant CD80 polypeptide is increased at least 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold 60-fold, 70-fold, 80-fold, 90-fold, 100-fold, 150-fold, 200-fold, 250-fold, 300-fold, 350-fold, 400-fold, or 450-fold compared to binding affinity of the unmodified CD80 for the ectodomain of PD-L1.

In some embodiments, the variant CD80 polypeptides provided herein that exhibit, mediate, or effect PD-L1-dependent CD28 costimulatory activity, retain binding to the ectodomain of CD28 compared to a wild-type or unmodified CD80. For example the variant CD80 polypeptide can retain at least or about at least 2%, 3%, 4%, 5%, 6%, 7%, 8,%, 9%, 10%, 12%, 15%, 20%, 25%, 30%, 35%, 40%, 50%, 55%, 60%, 65%, 70% 75%, 80%, 85%, 90%, or 95% of the affinity to the ectodomain of CD28, compared to the binding affinity of the unmodified CD80 polypeptide for the ectodomain of CD28.

In some embodiments, the variant CD80 polypeptides provided herein that exhibit, mediate, or effect PD-L1-dependent CD28 costimulatory activity exhibit increased affinity to the ectodomain of CD28, compared to the binding affinity of the unmodified CD80 for the ectodomain of CD28. For example, the variant CD80 polypeptide can exhibit increased affinity to the ectodomain of CD28 that is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 150-fold, or 200-fold, compared to binding affinity of the unmodified CD80 for the ectodomain of CD28. 3. CTLA-4

In some embodiments, the variant CD80 polypeptide exhibits increased affinity for the ectodomain of CTLA-4 compared to a wild-type or unmodified CD80 polypeptide, such as a wildtype or unmodified CD80 polypeptide, comprising the sequence set forth in SEQ ID NO: 2, 76, 150, or 1245. In some embodiments, the increased affinity to the ectodomain of CTLA-4 is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold or 60-fold compared to binding affinity of the unmodified CD80 for the ectodomain of CTLA-4.

Non-limiting examples of CD80 variant polypeptides with altered (e.g. increased) binding to CTLA-4 are described in the examples. Exemplary binding activities for binding CTLA-4 are shown in a flow-cytometry based assay based on mean fluorescence intensity (MFI) and comparison of binding to the corresponding unmodified or wild-type CD80 polypeptide. Among such variant polypeptides are polypeptides that exhibit an increase binding for CTLA-4, e.g. human CTLA-4, as described. Further, non-limiting examples of CD80 variant polypeptides with altered (e.g. increased) signaling induced following interactions with one or more functional binding partners, e.g. CTLA-4, are described in the examples. Exemplary functional activities are shown, in some aspects, in an mixed lymphocyte reaction and/or reporter-based assay based on changed in fluorescence of a reporter in a T cell reporter Jurkat cell line, including in comparison to the corresponding unmodified or wild-type CD80 polypeptide.

Among non-limiting examples of such variant polypeptide include, in some of these embodiments, the variant CD80 polypeptide that exhibits increased binding affinity for CTLA-4 compared to a wild-type or unmodified CD80 polypeptide has one or more amino acid modifications (e.g., substitutions) corresponding to positions 7, 12, 13, 16, 18, 20, 22, 23, 24, 26, 27, 30, 33, 35, 37, 38, 41, 42, 43, 44, 46, 47, 48, 52, 53, 54, 57, 58, 61, 62, 63, 67, 68, 69, 70, 71, 72, 73, 74, 77, 79, 81, 83, 84, 85, 87, 88, 89, 90, 91, 92, 93, 94, 95, and/or 97 of SEQ ID NO: 2, 76, 150, or 1245. In some of these embodiments, the variant CD80 polypeptide that exhibits increased binding affinity for CTLA-4 compared to a wild-type or unmodified CD80 polypeptide has one or more amino acid modifications (e.g., substitutions) corresponding to positions 7, 23, 26, 30, 35, 46, 57, 58, 71, 73, 79, and/or 84 of SEQ ID NO: 2, 76, 150, or 1245.

In some embodiments, the variant CD80 polypeptide has one or more amino acid substitutions selected from the group consisting of E7D, A12T, T13A, T13R, S15T, C16R, H18A, H18C, H18F, H18I, H18L, H18T, H18V, H18Y, V20I, S21P, V22A, V22D, V22L, E23D, E23G, E24D, A26D, A26E, A26G, A26H, A26K, A26N, A26P, A26Q, A26R, A26S, A26T, Q27H, Q27L, Q27R, I30V, Q33L, Q33R, E35D, E35G, K37E, M38I, M38T, M38V, T41S, M42V, M43I, M43L, M43T, M43V, S44P, D46E, D46N, D46V, M47I, M47L, M47T, M47V, M47Y, N48D, N48H, N48K, N48R, N48S, N48T, N48Y, E52D, Y53F, Y53H, K54E, K54R, T57A, T57I, I58V, I61F, I61N, I61V, T62A, T62N, T62S, N63D, N64S, I67L, I67T, V68E, V68I, V68L, V68M, I69F, L70M, L70Q, L70R, A71D, A71G, L72P, L72V, R73H, P4S, E77A, T79I, T79M, E81G, E81K, V83I, V84I, L85M, L85Q, Y87C, Y87D, Y87N, E88D, E88V, K89N, D90G, D90N, D90P, A91G, A91S, A91V, F92V, F92Y, K93E, K93R, K93T, R94L, R94Q, R94W, E95D, E95K, E95V, L97Q, and L97R. In some embodiments, the variant CD80 polypeptide has one or more amino acid substitutions selected from the group consisting of E7D, T13A, T13R, S15T, C16R, H18A, H18C, H18F, H18I, H18T, H18V, V20I, V22D, V22L, E23D, E23G, E24D, A26D, A26E, A26G, A26H, A26K, A26N, A26P, A26Q, A26R, A26S, A26T, Q27H, Q27L, I30V, Q33L, Q33R, E35D, E35G, T41S, M42V, M43L, M43T, D46E, D46N, D46V, M47I, M47L, M47V, M47Y, N48D, N48H, N48K, N48R, N48S, N48T, N48Y, Y53F, K54E, K54R, T57A, T57I, I58V, I61F, I61V, T62A, T62N, I67L, V68E, V68I, V68L, I69F, L70M, A71D, A71G, L72V, R73H, P4S, T79I, T79M, E81G, E81K, V84I, L85M, L85Q, Y87C, Y87D, E88V, D90P, F92V, R94Q, R94W, E95D, E95V, and L97Q.

In some embodiments, the one or more amino acid substitution is Q27H/T41S/A71D, T13R/C16R/L70Q/A71D, T57I, V22L/M38V/M47T/A71D/L85M, S44P/167T/P74S/E81G/E95D, A71D, T13A/I61N/A71D, E35D/M47I, M47V/N48H, V20I/M47V/T57I/V84I, V20I/M47V/A71D, A71D/L72V/E95K, V22L/E35G/A71D/L72P, E35D/A71D, E35D/I67L/A71D, Q27H/E35G/A71D/L72P/T79I, T13R/M42V/M47/A71D, E35D, E35D/M47I/L70M, E35D/A71D/L72V, E35D/M43L/L70M, A26P/E35D/M43I/L85Q/E88D, E35D/D46V/L85Q, Q27L/E35D/M47/T57I/L70Q/E88D, M47V/I69F/A71D/V83I, E35D/T57A/A71D/L85Q, H18Y/A26T/E35D/A71D/L85Q, E35D/M47L, E23D/M42V/M43I/I58V/L70R, V68M/L70M/A71D/E95K, E35D/M43I/A71D, T41S/T57I/L70R, H18Y/A71D/L72P/E88V, V20I/A71D, E23G/A26S/E35D/T62N/A71D/L72V/L85M, A12T/E24D/E35D/D46V/I61V/L72P/E95V, E35G/K54E/A71D/L72P, L70Q/A71D, A26E/E35D/M47L/L85Q, D46E/A71D, E35D/M47L/L85Q, H18Y/E35D/M47L, A26E/E35D/M43T/M47L/L85Q/R94Q, E24D/Q33L/E35D/M47V/K54R/L85Q, E7D/E35D/M47I/L97Q, H18L/V22A/E35D/M47L/N48T/L85Q, Q27H/E35D/M47L/L85Q/R94Q/E95K, E35D/M47I/E77A/L85Q/R94W, V22A/E35D/V68E/A71D, E35D/M47L/A71G/L97Q, E35D/M47V/A71G/L85M/L97Q, E35D/D46E/M47V/L97Q, E35D/D46V/M47I/A71G/F92V, E35D/L85Q/K93T/E95V/L97Q, Q27H/E35D/M47I/L85Q/D90G, E35D/M47I/L85Q/D90G, E35D/M47I/T62S/L85Q, A26E/E35D/M47L/A71G, V22A/E35D/M47I/Y87N, H18Y/A26E/E35D/M47L/L85Q/D90G, E35D/M47V/A71G/E88D, E35D/A71G, E35D/M47V/A71G, I30V/E35D/M47V/A71G/A91V, V22D/E35D/M47L/L85Q, H18Y/E35D/N48K, E35D/T41S/M47V/A71G/K89N, E35D/M47V/N48T/L85Q, E35D/D46E/M47V/A71D/D90G, E35D/D46E/M47V/A71D, E35D/T41S/M43I/A71G/D90G, E35D/T41S/M43I/M47V/A71G, E35D/T41S/M43I/M47L/A71G, H18Y/Y22A/E35D/M47V/T62S/A71G, H18Y/A26E/E35D/M47L/V68M/A71G/D90G, E35D/K37E/M47V/N48D/L85Q/D90N, E35D/D46V/M47L/V68M/L85Q/E88D, E35D/T41S/M43V/M47I/L70M/A71G, E35D/D46E/M47V/N63D/L85Q, E35D/M47V/T62A/A71D/K93E, E35D/D46E/M47V/V68M/D90G/K93E, E35D/M43I/M47V/K89N, E35D/M47L/A71G/L85M/F92Y, E35D/M42V/M47V/E52D/L85Q, E35D/T41S/M47V/L97Q, E35D/Y53H/A71G/D90G/L97R, E35D/A71D/L72V/R73H/E81K, E35D/M38T/D46E/M47V/N48S, E35D/M38T/M43V/M47V/N48R/L85Q, E35D/N48K/L72V, E35D/T41S/N48T, D46V/M47I/A71G, M47I/A71G, E35D/M43I/M47L/L85M, E35D/M43I/D46E/A71G/L85M, H18Y/E35D/M47L/A71G/A91S, E35D/M47I/N48K/I61F, E35D/M47V/T62S/L85Q, M43I/M47L/A71G, E35D/M47V, E35D/M47L/A71G/L85M, V22A/E35D/M47L/A71G, E35D/M47L/A71G, E35D/D46E/M47I, Q27H/E35D/M47I, E35D/D46E/L85M, E35D/D46E/A91G, E35D/D46E, E35D/L97R, H18Y/E35D, Q27L/E35D/M47V/I61V/L85M, E35D/M47V/I61V/L85M, E35D/M47V/L85M/R94Q, E35D/M47V/N48K/L85M, H18Y/E35D/M47V/N48K, A26E/Q27R/E35D/M47L/N48Y/L85Q, E35D/M47I/T62S/L85Q/E88D, E24D/Q27R/E35D/T41S/M47V/L85Q, S15T/H18Y/E35D/M47V/T62A/N64S/A71G/L85Q/D90N, E35D/M47L/V68M/A71G/L85Q/D90G, H18Y/E35D/M47I/V68M/A71G/R94L, H18Y/Y22A/E35D/T41S/M47V/T62N/A71G/A91G, E24D/E35D/M47L/V68M/E95V/L97Q, E35D/D46E/M47I/T62A/V68M/L85M/Y87C, E35D/D46E/M47I/V68M/L85M, E35D/D46E/M47L/V68M/A71G/Y87C/K93R, E35D/D46E/M47L/V68M/T79M/L85M, E35D/D46E/M47V/V68M/L85Q, E35D/M43I/M47L/V68M, E35D/M47I/V68M/Y87N, E35D/M47L/V68M/E95V/L97Q, E35D/M47L/Y53F/V68M/A71G/K93R/E95V, E35D/M47V/N48K/V68M/A71G/L85M, E35D/M47V/N48K/V68M/L85M, E35D/M47V/V68M/L85M, E35D/M47V/V68M/L85M/Y87D, E35D/T41S/D46E/M47I/V68M/K93R/E95V, H18Y/E35D/D46E/M47I/V68M/R94L, H18Y/E35D/D46E/M47I/V68M/R94L, H18Y/E35D/M47I/V68M/Y87N, H18Y/E35D/M47I/V68M/Y87N, H18Y/E35D/M47L/V68M/A71G/L85M, H18Y/E35D/M47L/V68M/A71G/L85M, H18Y/E35D/M47L/V68M/E95V/L97Q, H18Y/E35D/M47L/V68M/E95V/L97Q, H18Y/E35D/M47L/Y53F/V68M/A71G, H18Y/E35D/M47L/Y53F/V68M/A71G/K93R/E95V, H18Y/E35D/M47V/V68M/L85M, H18Y/E35D/V68M/A71G/R94Q/E95V, H18Y/E35D/V68M/L85M/R94Q, H18Y/E35D/V68M/T79M/L85M, H18Y/V22D/E35D/M47V/N48K/V68M, S21P/E35D/K37E/D46E/M47I/V68M, S21P/E35D/K37E/D46E/M47I/V68M/R94L, T13R/E35D/M47L/V68M, T13R/Q33R/E35D/M38I/M47L/V68M/E95V/L97Q, T13R/Q33R/E35D/M38I/M47L/V68M/L85M, T13R/Q33R/E35D/M38I/M47L/V68M/L85M/R94Q, T13R/Q33R/E35D/M47L/V68M, T13R/Q33R/E35D/M47L/V68M/L85M, V22D/E24D/E35D/M47L/V68M, V22D/E24D/E35D/M47L/V68M/L85M/D90G, V22D/E24D/E35D/M47V/V68M, H18Y/E35D/M47V/V68M/A71G, H18C/A26P/E35D/M47L/V68M/A71G, H18I/A26P/E35D/M47V/V68M/A71G, H18L/A26N/D46E/V68M/A71G/D90G, H18L/E35D/M47V/V68M/A71G/D90G, H18T/A26N/E35D/M47L/V68M/A71G, H18V/A26K/E35D/M47L/V68M/A71G, H18V/A26N/E35D/M47V/V68M/A71G, H18V/A26P/E35D/M47V/V68L/A71G, H18V/A26P/E35D/M47L/V68M/A71G, H18V/E35D/M47V/V68M/A71G/D90G, H18Y/A26P/E35D/M47U/V68M/A71G, H18Y/A26P/E35D/M47V/V68M/A71G, H18Y/E35D/M47V/V68L/A71G/D90G, H18Y/E35D/M47V/V68M/A71G/D90G, A26P/E35D/M47I/V68M/A71G/D90G, H18V/A26G/E35D/M47V/V68M/A71G/D90G, H18V/A26S/E35D/M47L/V68M/A71G/D90G, H18V/A26R/E35D/M47L/V68M/A71G/D90G, H18V/A26D/E35D/M47V/V68M/A71G/D90G, H18V/A26Q/E35D/M47V/V68L/A71G/D90G, H18A/A26P/E35D/M47L/V68M/A71G/D90G, H18A/A26N/E35D/M47L/V68M/A71G/D90G, H18F/A26P/E35D/M47I/V68M/A71G/D90G, H18F/A26H/E35D/M47L/V68M/A71G/D90G, H18F/A26N/E35D/M47V/V68M/A71G/D90K, H18Y/A26N/E35D/M47F/V68M/A71G/D90G, H18Y/A26P/E35D/M47V/V68U/A71G/D90G, H18Y/A26Q/E35D/M47T/V68M/A71G/D90G, H18R/A26P/E35D/D46N/M47V/V68M/A71G/D90P, or H18F/A26D/E35D/D46E/M47T/V68M/A71G/D90G.

In some embodiments, the variant CD80 polypeptides provided herein, that exhibit increased affinity for the ectodomain of CTLA-4, compared to a wild-type or unmodified CD80 polypeptide, results in decreased inhibitory signal from the CTLA-4 inhibitory receptor. In some embodiments, the variant CD80 polypeptides provided herein blocks interaction of CD80 with CTLA-4, thereby blocking the CTLA-4 inhibitory receptor. In some embodiments, a variant CD80 polypeptide that inhibits or decreases the activity of the inhibitory receptor CTLA-4 will produce a signal that is 90%, 85%, 80%, 75%, 70%, 65%, 60%, 55%, 50%, 45%, 40%, 35%, 30%, 25%, 20%, 15%, 10%, 5%, or less, of the CTLA-4 inhibitory signal in the presence of the wild-type or unmodified CD80 polypeptide. In such examples, the wild-type or unmodified CD80 polypeptide has the same sequence as the variant CD80 polypeptide except that it does not contain the one or more amino acid modifications (e.g., substitutions).

B. Multimerization Domains

The variant CD80 IgSF domain fusion proteins comprising a variant CD80 provided herein in which is contained a vIgD can be formatted in a variety of ways as a soluble protein. In some embodiments, the variant CD80 IgSF domain fusion protein contains a multimerization domain. In some embodiments, the multimerization domain is an Fc region. In some particular aspects, the Fc region is an effector Fc capable of binding the FcR and/or mediating one or more effector activity. In other particular aspects, the Fc region is an Fc that is modified by one or more amino acid substitutions to reduce effector activity or to render the Fc inert for Fc effector function.

In some embodiments, the variant CD80 IgSF domain fusion protein agonizes or stimulates activity of its binding partner, e.g., CD28. In some embodiments, agonism of CD28 may be useful to promote immunity in oncology. In some cases, a variant CD80 IgSF domain fusion protein comprising a variant CD80 polypeptide is provided to antagonize or block activity of its binding partner, e.g., CTLA-4 and/or PD-L1. In some embodiments, antagonism of CTLA-4 or PD-L1/PD-1 may be useful to promote immunity in oncology. In some embodiments, agonism of CD28 can be dependent on or enhanced by CD80 binding of PD-L1. Such PD-L1-dependent agonism of CD28 may be useful to promote immunity in oncology. A skilled artisan can readily determine the activity of a particular format, such as for antagonizing or agonizing one or more specific binding partner. Exemplary methods for assessing such activities are provided herein, including in the examples.

In some embodiments, the immunomodulatory protein containing a variant CD80 polypeptide is a soluble protein. Those of skill will appreciate that cell surface proteins typically have an intracellular, transmembrane, and extracellular domain (ECD) and that a soluble form of such proteins can be made using the extracellular domain or an immunologically active subsequence thereof. Thus, in some embodiments, the immunomodulatory protein containing a variant CD80 polypeptide lacks a transmembrane domain or a portion of the transmembrane domain. In some embodiments, the immunomodulatory protein containing a variant CD80 lacks the intracellular (cytoplasmic) domain or a portion of the intracellular domain. In some embodiments, the immunomodulatory protein containing the variant CD80 polypeptide only contains the vIgD portion containing the ECD domain or a portion thereof containing an IgV domain and/or IgC (e.g., IgC2) domain or domains or specific binding fragments thereof containing the amino acid modification(s).

In some aspects, provided are variant CD80 IgSF domain fusion proteins comprising a vIgD of CD80 that is fused to a multimerization domain, e.g. an Fc chain. Those of skill will appreciate that cell surface proteins typically have an intracellular, transmembrane, and extracellular domain (ECD) and that a soluble form of such proteins can be made using the extracellular domain or an immunologically active subsequence thereof. Thus, in some embodiments, the immunomodulatory protein containing a variant CD80 polypeptide lacks a transmembrane domain or a portion of the transmembrane domain. In some embodiments, the immunomodulatory protein containing a variant CD80 lacks the intracellular (cytoplasmic) domain or a portion of the intracellular domain. In some embodiments, the immunomodulatory protein containing the variant CD80 polypeptide only contains the vIgD portion containing the ECD domain or a portion thereof containing an IgV domain and/or IgC (e.g., IgC2) domain or domains or specific binding fragments thereof containing the amino acid modification(s).

In some embodiments, a variant CD80 IgSF domain fusion protein comprising a variant CD80 can include one or more variant CD80 polypeptides of the invention. In some embodiments a polypeptide of the invention will comprise exactly 1, 2, 3, 4, 5 variant CD80 sequences. In some embodiments, at least two of the variant CD80 sequences are identical variant CD80 sequences.

In some embodiments, the provided variant CD80 IgSF domain fusion protein comprises two or more vIgD sequences of CD80. In some embodiments, the provided variant CD80 IgSF domain fusion protein comprises three or more vIgD sequences of CD80. In some embodiments, the variant CD80 IgSF domain fusion protein exhibits multivalent binding to its binding partner. For example, in some cases, the variant CD80 IgSF domain fusion protein exhibits bivalent, trivalent, tetravalent, pentavalent, or hexavalent binding to its binding partner. In some embodiments, the provided variant CD80 IgSF domain fusion protein is bivalent. In some embodiments, the provided variant CD80 IgSF domain fusion protein is tetravalent.

In some embodiments, multiple variant CD80 polypeptides within the polypeptide chain can be identical (i.e., the same species) to each other or be non-identical (i.e., different species) variant CD80 sequences. In addition to single polypeptide chain embodiments, in some embodiments two, three, four, or more of the polypeptides of the invention can be covalently or non-covalently attached to each other. Thus, monomeric, dimeric, and higher order (e.g., 3, 4, 5, or more) multimeric proteins are provided herein. For example, in some embodiments exactly two polypeptides of the invention can be covalently or non-covalently attached to each other to form a dimer. In some embodiments, attachment is made via interchain cysteine disulfide bonds. Compositions comprising two or more polypeptides of the invention can be of an identical species or substantially identical species of polypeptide (e.g., a homodimer) or of non-identical species of polypeptides (e.g., a heterodimer). A composition having a plurality of linked polypeptides of the invention can, as noted above, have one or more identical or non-identical variant CD80 polypeptides of the invention in each polypeptide chain.

In some embodiments, the immunomodulatory protein contains a variant CD80 polypeptide that is linked, directly or indirectly via a linker to a multimerization domain. For example, the variant CD80 IgSF domain fusion proteins provided herein can be formatted as multimeric (e.g. dimeric, trimeric, tetrameric, or pentameric) molecules. In some aspects, the multimerization domain increases the half-life of the molecule. Interaction of two or more variant CD80 polypeptides can be facilitated by their linkage, either directly or indirectly, to any moiety or other polypeptide that are themselves able to interact to form a stable structure. For example, separate encoded variant CD80 polypeptide chains can be joined by multimerization, whereby multimerization of the polypeptides is mediated by a multimerization domain. Typically, the multimerization domain provides for the formation of a stable protein-protein interaction between a first variant CD80 polypeptide and a second variant CD80 polypeptide.

Homo- or heteromultimeric polypeptides can be generated from co-expression of separate variant CD80 polypeptides. The first and second variant CD80 polypeptides can be the same or different. In particular embodiments, the first and second variant CD80 polypeptides are the same in a homodimer, and each is linked to a multimerization domain that is the same. In other embodiments, heterodimers can be formed by linking first and second variant CD80 polypeptides that are different. In some of such embodiments, the first and second variant CD80 polypeptide are linked to different multimerization domains capable of promoting heterodimer formation.

In some embodiments, a multimerization domain includes any capable of forming a stable protein-protein interaction. The multimerization domains can interact via an immunoglobulin sequence (e.g. Fc domain; see e.g., International Patent Pub. Nos. WO 93/10151 and WO 2005/063816 US; U.S. Pub. No. 2006/0024298; U.S. Pat. No. 5,457,035); leucine zipper (e.g., from nuclear transforming proteins fos and jun or the proto-oncogene c-myc or from General Control of Nitrogen (GCN4)) (see e.g., Busch and Sassone-Corsi (1990) Trends Genetics, 6:36-40; Gentz et al., (1989) Science, 243:1695-1699); a hydrophobic region; a hydrophilic region; or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. Pat. No. 5,731,168; International Patent Pub. Nos. WO 98/50431 and WO 2005/063816; Ridgway et al. (1996) Protein Engineering, 9:617-621. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or tryptophan). Compensatory cavities of identical or similar size to the protuberances are optionally created on the interface of the second polypeptide by replacing large amino acid side chains with smaller ones (e.g., alanine or threonine). Exemplary multimerization domains are described below.

The variant CD80 polypeptide can be joined anywhere, but typically via its N- or C-terminus, to the N- or C-terminus of a multimerization domain to form a chimeric polypeptide. The linkage can be direct or indirect via a linker. The chimeric polypeptide can be a fusion protein or can be formed by chemical linkage, such as through covalent or non-covalent interactions. For example, when preparing a chimeric polypeptide containing a multimerization domain, nucleic acid encoding all or part of a variant CD80 polypeptide can be operably linked to nucleic acid encoding the multimerization domain sequence, directly or indirectly or optionally via a linker domain. In some cases, the construct encodes a chimeric protein where the C-terminus of the variant CD80 polypeptide is joined to the N-terminus of the multimerization domain. In some instances, a construct can encode a chimeric protein where the N-terminus of the variant CD80 polypeptide is joined to the C-terminus of the multimerization domain.

In some embodiments, the variant CD80 IgSF domain fusion protein comprises two or more polypeptides joined by multimerization, such as joined as dimeric, trimeric, tetrameric, or pentameric molecules. In some embodiments of the configurations, the variant CD80 IgSF domain fusion proteins containing one or more variant CD80 polypeptides are fused to a multimerization domain. In some examples, the variant CD80 IgSF domain fusion proteins containing one or more variant CD80 polypeptides (e.g., comprising separate encoded polypeptide chains) are fused with a sequence of amino acids that promotes dimerization, trimerization, tetramerization, or pentamerization of the proteins.

In some embodiments, the variant CD80 IgSF domain fusion proteins containing one or more variant CD80 polypeptides (e.g., separate encoded polypeptide chains) are fused with a sequence of amino acids that promotes pentamerization of the proteins. In some embodiments, the variant CD80 IgSF domain fusion proteins containing one or more variant CD80 polypeptides (e.g., separate encoded polypeptide chains) are fused to a portion of the cartilage oligomeric matrix protein (COMP) assembly domain (Voulgaraki et al., Immunology (2005) 115(3):337-346. In some examples, the COMP is or contains an amino acid sequence as set forth in SEQ ID NO: 1524 (e.g. amino acids 29-72 of the full length COMP, Uniprot accession number P49747) or a sequence that has 85%, 85%, 87%, 88%, 89%, 90%, 91%, 92%, 93%, 94%, 95%, 96%, 97%, 98%, 99% or more sequence identity to SEQ ID NO: 1524.

In some embodiments, the variant CD80 IgSF domain fusion proteins containing one or more variant CD80 polypeptides (e.g., separate encoded polypeptide chains) are fused with a sequence of amino acids that promotes tetramerization of the proteins. In some embodiments, the variant CD80 IgSF domain fusion proteins containing one or more variant CD80 polypeptides (e.g., separate encoded polypeptide chains) are fused to a vasodilator-stimulated phosphoprotein (VASP) tetramerization domain (Bachmann et al., J Biol Chem (1999) 274(33):23549-23557). In some embodiments, the VASP is or contains an amino acid sequence as set forth in SEQ ID NO: 1525 (e.g. amino acids 343-375 of the full length VASP; Uniprot accession number P50552) or a sequence that has 85%, 85%, 87%, 88%, 89%, 90%, 91%, 92%, 93%, 94%, 95%, 96%, 97%, 98%, 99% or more sequence identity to SEQ ID NO: 1525.

In some embodiments, the variant CD80 IgSF domain fusion proteins containing one or more variant CD80 polypeptides (e.g., separate encoded polypeptide chains) are fused with a sequence of amino acids that promotes trimerization of the proteins. In some embodiments, the variant CD80 IgSF domain fusion proteins containing one or more variant CD80 polypeptides (e.g., separate encoded polypeptide chains) are fused to a ZymoZipper (ZZ) 12.6 domain. In some embodiments, the ZZ domain is or contains an amino acid sequence as set forth in SEQ ID NO: 1526 (See U.S. Pat. No. 7,655,439) or a sequence that has 85%, 85%, 87%, 88%, 89%, 90%, 91%, 92%, 93%, 94%, 95%, 96%, 97%, 98%, 99% or more sequence identity to SEQ ID NO: 1526.

In some embodiments, the variant CD80 IgSF domain fusion protein is tetravalent. In some embodiments, the variant CD80 IgSF domain fusion protein contains two copies of a variant CD80 IgSf domain. In some embodiments, the variant CD80 IgSF domain fusion protein comprises the components variant CD80 IgSf domain(s), linker(s), and multimerization domain in various order and combinations. In some embodiments, the variant CD80 IgSF domain fusion protein comprises the following in the order: variant CD80 IgSF domain—linker—multimerization domain—linker—variant CD80 IgSf domain. In some embodiments, the variant CD80 IgSF domain fusion protein comprises the following in the order: variant CD80 IgSF domain—linker—variant CD80 IgSf domain—linker—multimerization domain. In some embodiments, the variant CD80 IgSF domain fusion protein comprises the following in the order: multimerization domain—linker—variant CD80 IgSF domain—variant CD80 IgSf domain. Exemplary variant CD80 IgSF domain fusion proteins are shown in FIG. 3. In some embodiments, the variant CD80 IgSF domain fusion protein can further contain a third CD80 vIgD. In some embodiments, the CD80 vIgD(s) are independently linked, directly or indirectly, to the N- or C-terminus of an Fc region or to the N- or C-terminus of another CD80 vIgD.

A polypeptide multimer contains multiple, such as two, chimeric proteins created by linking, directly or indirectly, two of the same or different variant CD80 polypeptides directly or indirectly to a multimerization domain. In some examples, where the multimerization domain is a polypeptide, a gene fusion encoding the variant CD80 polypeptide and multimerization domain is inserted into an appropriate expression vector. The resulting chimeric or fusion protein can be expressed in host cells transformed with the recombinant expression vector, and allowed to assemble into multimers, where the multimerization domains interact to form multivalent polypeptides. Chemical linkage of multimerization domains to variant CD80 polypeptides can be carried out using heterobifunctional linkers.

The resulting chimeric polypeptides, such as fusion proteins, and multimers formed therefrom, can be purified by any suitable method such as, for example, by affinity chromatography over Protein A or Protein G columns. Where two nucleic acid molecules encoding different polypeptides are transformed into cells, formation of homo- and heterodimers will occur. Conditions for expression can be adjusted so that heterodimer formation is favored over homodimer formation.

In some embodiments, the multimerization domain is an Fc domain or portions thereof from an immunoglobulin. In some embodiments, the variant CD80 IgSF domain fusion protein comprises one or more variant CD80 polypeptide(s) attached to an immunoglobulin Fc (yielding an “immunomodulatory Fc fusion,” such as a “variant CD80-Fc fusion,” also termed a CD80 vIgD-Fc fusion). In some embodiments, the attachment of the variant CD80 polypeptide(s) is at the N-terminus of the Fc. In some embodiments, the attachment of the variant CD80 polypeptide (s) is at the C-terminus of the Fc. In some embodiments, two or more CD80 variant polypeptides (the same or different) are independently attached at the N-terminus and at the C-terminus.

In some embodiments, the one or more variant CD80 polypeptide(s) can be joined anywhere, but typically via its N- or C-terminus, to the N- or C-terminus of a multimerization domain to form a chimeric polypeptide. The linkage can be direct or indirect via a linker. Also, the chimeric polypeptide can be a fusion protein or can be formed by chemical linkage, such as through covalent or non-covalent interactions. For example, when preparing a chimeric polypeptide containing a multimerization domain, nucleic acid encoding one or more variant CD80 polypeptide(s) can be operably linked to nucleic acid encoding the multimerization domain sequence, directly or indirectly or optionally via a linker domain. In some cases, the construct encodes a chimeric protein where the C-terminus of the variant CD80 polypeptide is joined to the N-terminus of the multimerization domain. In some instances, a construct can encode a chimeric protein where the N-terminus of the variant CD80 polypeptide is joined to the N- or C-terminus of the multimerization domain.

In some embodiments, the one or more variant CD80 polypeptides are positioned N-terminal to the multimerization domain. In some embodiments, two variant CD80 polypeptide(s) are positioned N-terminal to the multimerization domain. In some embodiments, the one or more variant CD80 polypeptide(s) are positioned C-terminal to the multimerization domain. In some embodiments, two variant CD80 polypeptides are positioned C-terminal to the multimerization domain.

In some embodiments, each of the multimerization domain is linked to two or more variant CD80 polypeptides to form a chimeric polypeptide. In some cases, the construct encodes a chimeric protein where the C-terminus of the first variant CD80 polypeptide is joined to the N-terminus of a second variant CD80 polypeptide and the C-terminus of the second variant CD80 polypeptide is joined to N-terminus of the multimerization domain. In some embodiments, the construct encodes a chimeric protein where the C-terminus of the multimerization domain is joined to the N-terminus of the first variant CD80 polypeptide and the C-terminus of the first variant CD80 polypeptide is joined to the N-terminus of the second variant CD80 polypeptide. In some embodiments, the construct encodes a chimeric protein where the C-terminus of the first variant CD80 polypeptide is joined the the N-terminus of the multimerization domain and C-terminus of the multimerization domain is joined to the the N-terminus of the second variant CD80 polypeptide. In some embodiments, the multimerization domain is an Fc domain or portions thereof from an immunoglobulin.

In some embodiments, the first and the second variant CD80 polypeptide are the same. In some embodiments, the first and the second variant CD80 polypeptides are different. In some embodiments, the chimeric polypeptide further contains a third CD80 polypeptide joined either N-terminal or C-terminal to the polypeptides described.

In some embodiments, the variant CD80 IgSF domain fusion protein comprises two or more polypeptides joined by multimerization, such as joined as dimeric, trimeric, tetrameric, or pentameric molecules, each polypeptide having the configuration of the chimeric polypeptides containing one or more variant CD80 polypeptides as described.

In some embodiments, the Fc is murine or human Fc. In some embodiments, the Fc is a mammalian or human IgG1, lgG2, lgG3, or lgG4 Fc regions. In some embodiments, the Fc is derived from IgG1, such as human IgG1. In some embodiments, the Fc comprises the amino acid sequence set forth in SEQ ID NO: 1502, 1510, or 1518 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 1502, 1510, or 1518.

In some embodiments, the Fc region contains one more modifications to alter (e.g., reduce) one or more of its normal functions. In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments, such functions can be reduced or altered in an Fc for use with the provided Fc fusion proteins.

In some embodiments, one or more amino acid modifications may be introduced into the Fc region of a CD80-Fc variant fusion provided herein, thereby generating an Fc region variant. In some embodiments, the Fc region variant has decreased effector function. There are many examples of changes or mutations to Fc sequences that can alter effector function. For example, WO 00/42072, WO2006019447, WO2012125850, WO2015/107026, US2016/0017041 and Shields et al. J Biol. Chem. 9(2): 6591-6604 (2001) describe exemplary Fc variants with improved or diminished binding to FcRs. The contents of those publications are specifically incorporated herein by reference.

In some embodiments, the provided variant CD80-Fc fusions comprise an Fc region that exhibits reduced effector functions, which makes it a desirable candidate for applications in which the half-life of the CD80-Fc variant fusion in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the CD80-Fc variant fusion lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTFI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the CD80-Fc variant fusion is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Variant CD80 IgSF domain fusion proteins with reduced effector function include those with substitution of one or more of Fc region residues 238, 265, 269, 270, 297, 327 and 329 by EU numbering (U.S. Pat. No. 6,737,056). Such Fc mutants include Fc mutants with substitutions at two or more of amino acid positions 265, 269, 270, 297 and 327 by EU numbering, including the so-called “DANA” Fc mutant with substitution of residues 265 and 297 to alanine (U.S. Pat. No. 7,332,581).

In some embodiments, the Fc region of variant CD80 IgSF domain fusion proteins has an Fc region in which any one or more of amino acids at positions 234, 235, 236, 237, 238, 239, 270, 297, 298, 325, and 329 (indicated by EU numbering) are substituted with different amino acids compared to the native Fc region. Such alterations of Fc region are not limited to the above-described alterations, and include, for example, alterations such as deglycosylated chains (N297A and N297Q), IgG1-N297G, IgG1-L234A/L235A, IgG1-L234A/L235E/G237A, IgG1-A325A/A330S/P331S, IgG1-C226S/C229S, IgG1-C226S/C229S/E233P/L234V/L235A, IgG1-E233P/L234V/L235A/G236del/S267K, IgG1-L234F/L235E/P331S, IgG1-S267E/L328F, IgG2-V234A/G237A, IgG2-H268Q/V309L/A330S/A331S, IgG4-L235A/G237A/E318A, and IgG4-L236E described in Current Opinion in Biotechnology (2009) 20 (6), 685-691; alterations such as G236R/L328R, L235G/G236R, N325A/L328R, and N325LL328R described in WO 2008/092117; amino acid insertions at positions 233, 234, 235, and 237 (indicated by EU numbering); and alterations at the sites described in WO 2000/042072.

Certain Fc variants with improved or diminished binding to FcRs are described. (See, e.g., U.S. Pat. No. 6,737,056; WO 2004/056312, WO2006019447 and Shields et al., J. Biol. Chem. 9(2): 6591-6604 (2001).)

In some embodiments, there is provided a variant CD80 IgSF domain fusion protein comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.) or WO2015107026. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434 by EU numbering, e.g., substitution of Fc region residue 434 (U.S. Pat. No. 7,371,826).

In some embodiments, the Fc region of a CD80-Fc variant fusion comprises one or more amino acid substitution E356D and M358L by EU numbering. In some embodiments, the Fc region of a CD80-Fc variant fusion comprises one or more amino acid substitutions C220S, C226S and/or C229S by EU numbering. In some embodiments, the Fc region of a CD80 variant fusion comprises one or more amino acid substitutions R292C and V302C. See also Duncan & Winter, Nature 322:738-40 (1988); U.S. Pat. Nos. 5,648,260; 5,624,821; and WO 94/29351 concerning other examples of Fc region variants.

In some embodiments, the wild-type IgG1 Fc can be the Fc set forth in SEQ ID NO: 1502 having an allotype containing residues Glu (E) and Met (M) at positions 356 and 358 by EU numbering (e.g., f allotype). In other embodiments, the wild-type IgG1 Fc contains amino acids of the human G1m1 allotype, such as residues containing Asp (D) and Leu (L) at positions 356 and 358, e.g. as set forth in SEQ ID NO:1527. Thus, in some cases, an Fc provided herein can contain amino acid substitutions E356D and M358L to reconstitute residues of allotype G1 ml (e.g., alpha allotype). In some aspects, a wild-type Fc is modified by one or more amino acid substitutions to reduce effector activity or to render the Fc inert for Fc effector function. Exemplary effectorless or inert mutations include those described herein. Among effectorless mutations that can be included in an Fc of constructs provided herein are L234A, L235E and G237A by EU numbering. In some embodiments, a wild-type Fc is further modified by the removal of one or more cysteine residue, such as by replacement of the cysteine residues to a serine residue at position 220 (C220S) by EU numbering. Exemplary inert Fc regions having reduced effector function are set forth in SEQ ID NO: 1508 and SEQ ID NO: 1518, which are based on allotypes set forth in SEQ ID NO: 1502 or SEQ ID NO: 1527, respectively. In some embodiments, an Fc region used in a construct provided herein can further lack a C-terminal lysine residue.

In some embodiments, alterations are made in the Fc region that result in diminished C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided a CD80-Fc variant fusion comprising a variant Fc region comprising one or more amino acid modifications, wherein the variant Fc region is derived from IgG1, such as human IgG1. In some embodiments, the variant Fc region is derived from the amino acid sequence set forth in SEQ ID NO: 1502. In some embodiments, the Fc contains at least one amino acid substitution that is N82G by numbering of SEQ ID NO: 1502 (corresponding to N297G by EU numbering). In some embodiments, the Fc further contains at least one amino acid substitution that is R77C or V87C by numbering of SEQ ID NO: 1502 (corresponding to R292C or V302C by EU numbering). In some embodiments, the variant Fc region further comprises a C5S amino acid modification by numbering of SEQ ID NO: 1502 (corresponding to C220S by EU numbering), such as the Fc region set forth in SEQ ID NO: 1517. For example, in some embodiments, the variant Fc region comprises the following amino acid modifications: V297G and one or more of the following amino acid modifications C220S, R292C or V302C by EU numbering (corresponding to N82G and one or more of the following amino acid modifications C5S, R77C or V87C with reference to SEQ ID NO: 1502), e.g., the Fc region comprises the sequence set forth in SEQ ID NO: 1507. In some embodiments, the variant Fc region comprises one or more of the amino acid modifications C220S, L234A, L235E or G237A, e.g., the Fc region comprises the sequence set forth in SEQ ID NO: 1508. In some embodiments, the variant Fc region comprises one or more of the amino acid modifications C220S, L235P, L234V, L235A, G236del or S267K, e.g., the Fc region comprises the sequence set forth in SEQ ID NO: 1509. In some embodiments, the variant Fc comprises one or more of the amino acid modifications C220S, L234A, L235E, G237A, E356D or M358L, e.g., the Fc region comprises the sequence set forth in SEQ ID NO: 1513.

In some embodiments, CD80-Fc variant fusion provided herein contains a variant CD80 polypeptide in accord with the description set forth in Section I.A above. In some embodiments, there is provided a CD80-Fc variant fusion comprising any one of the described variant CD80 polypeptide linked to a variant Fc region, wherein the variant Fc region is not a human IgG1 Fc containing the mutations R292C, N297G and V302C (corresponding to R77C, N82G and V87C with reference to wild-type human IgG1 Fc set forth in SEQ ID NO: 1502). In some embodiments, there is provided a CD80-Fc variant fusion comprising any one of the variant CD80 polypeptide linked to an Fc region or variant Fc region, wherein the variant CD80 polypeptide is not linked to the Fc with a linker consisting of three alanines.

In some embodiments, the Fc region lacks the C-terminal lysine corresponding to position 232 of the wild-type or unmodified Fc set forth in SEQ ID NO: 1502 (corresponding to K447del by EU numbering). In some aspects, such an Fc region can additionally include one or more additional modifications, e.g., amino acid substitutions, such as any as described. Examples of such an Fc region are set forth in SEQ ID NO: 1508-1510, 1513, or 1519-1521.

In some embodiments, there is provided a CD80-Fc variant fusion comprising a variant Fc region in which the variant Fc comprises the sequence of amino acids set forth in any of SEQ ID NOS: 1513, 1508-1510, 1517, or 1519-1521 or a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1513, 1508-1510, 1517, or 1519-1521.

In some embodiments, the Fc is derived from IgG2, such as human IgG2. In some embodiments, the Fc comprises the amino acid sequence set forth in SEQ ID NO: 1503 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 1503.

In some embodiments, the Fc comprises the amino acid sequence set forth in SEQ ID NO: 1515 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 1515. In some embodiments, the IgG4 Fc is a stabilized Fc in which the CH3 domain of human IgG4 is substituted with the CH3 domain of human IgG1 and which exhibits inhibited aggregate formation, an antibody in which the CH3 and CH2 domains of human IgG4 are substituted with the CH3 and CH2 domains of human IgG1, respectively, or an antibody in which arginine at position 409 indicated in the EU index proposed by Kabat et al. of human IgG4 is substituted with lysine and which exhibits inhibited aggregate formation (see e.g., U.S. Pat. No. 8,911,726. In some embodiments, the Fc is an IgG4 containing the S228P mutation, which has been shown to prevent recombination between a therapeutic antibody and an endogenous IgG4 by Fab-arm exchange (see e.g., Labrijin et al. (2009) Nat. Biotechnol., 27(8): 767-71). In some embodiments, the Fc comprises the amino acid sequence set forth in SEQ ID NO: 1516 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 1516.

In some embodiments, the variant CD80 IgSF domain fusion protein is indirectly linked to the Fc sequence, such as via a linker. In some embodiments, one or more “peptide linkers” link the variant CD80 polypeptide and the Fc domain. In some embodiments, a peptide linker can be a single amino acid residue or greater in length. In some embodiments, the peptide linker has at least one amino acid residue but is no more than 20, 19, 18, 17, 16, 15, 14, 13, 12, 11, 10, 9, 8, 7, 6, 5, 4, 3, 2, or 1 amino acid residues in length. In some embodiments, the linker is a flexible linker. In some embodiments, the linker is (in one-letter amino acid code): GGGGS (“4GS” or “G₄S”; SEQ ID NO: 1523) or multimers of the 4GS linker, such as repeats of 2, 3, 4, or 5 4GS linkers, such as set forth in SEQ ID NO: 1505 (2×GGGS; (G₄S)₂) or SEQ ID NO: 1504 (3×GGGS; (G₄S)₃). In some embodiments, the linker can include a series of alanine residues alone or in addition to another peptide linker (such as a 4GS linker or multimer thereof). In some embodiments, the number of alanine residues in each series is 2, 3, 4, 5, or 6 alanines. In some embodiments, the linker is three alanines (AAA). In some embodiments, the variant CD80 polypeptide is indirectly linked to the Fc sequence via a linker, wherein the linker doe not consist of three alanines. In some examples, the linker is a 2×GGGS followed by three alanines (GGGGSGGGGSAAA; SEQ ID NO: 1506). In some embodiments, the linker can further include amino acids introduced by cloning and/or from a restriction site, for example the linker can include the amino acids GS (in one-letter amino acid code) as introduced by use of the restriction site BAMHI. For example, in some embodiments, the linker (in one-letter amino acid code) is GSGGGGS (SEQ ID NO:1522), GS(G₄S)₃ (SEQ ID NO: 1243), or GS(G₄S)₅ (SEQ ID NO: 1244). In some embodiments, the linker is a rigid linker. For example, the linker is an α-helical linker. In some embodiments, the linker is (in one-letter amino acid code): EAAAK or multimers of the EAAAK linker, such as repeats of 2, 3, 4, or 5 EAAAK linkers, such as set forth in SEQ ID NO: 1241 (1×EAAAK), SEQ ID NO: 1242 (3×EAAAK), or SEQ ID NO: 1251 (5×EAAAK). In some cases, the immunomodulatory polypeptide comprising a variant CD80 comprises various combinations of peptide linkers.

In some embodiments, the variant CD80 polypeptide of the variant CD80 IgSF domain fusion protein is directly linked to the Fc sequence. In some embodiments, the variant CD80 polypeptide is directly linked to an Fc, such as an inert Fc, that was additionally lacking all or a portion of the hinge region. An exemplary Fc, lacking a portion (6 amino acids) of the hinge region is set forth in SEQ ID NO: 1240. In some embodiments, where the CD80 polypeptide is directly linked to the Fc sequence, the CD80 polypeptide can be truncated at the C-terminus by 1, 2, 3, 4, 5, 6, 7, 8, 9, 10, 12, 13, 14, 15, or more amino acids. In some embodiments, the variant CD80 polypeptide is truncated to remove 1, 2, 3, 4, 5, 6, 7, 8, 9, 10, or more amino acids that connect the IgV region to the IgC region. For example, variant CD80 polypeptides can contain modifications in the exemplary wild-type CD80 backbone set forth in SEQ ID NO: 1245).

In some embodiments, the variant CD80 IgSF domain fusion protein (e.g. variant CD80-Fc fusion protein) is a dimer formed by two variant CD80 Fc polypeptides linked to an Fc domain. In some specific embodiments, identical or substantially identical species (allowing for 3 or fewer N-terminus or C-terminus amino acid sequence differences) of CD80-Fc variant fusion polypeptides will be dimerized to create a homodimer. In some embodiments, the dimer is a homodimer in which the two variant CD80 Fc polypeptides are the same. Alternatively, different species of CD80-Fc variant fusion polypeptides can be dimerized to yield a heterodimer. Thus, in some embodiments, the dimer is a heterodimer in which the two variant CD80 Fc polypeptides are different.

Also provided are nucleic acid molecules encoding the variant CD80-Fc fusion protein. In some embodiments, for production of an Fc fusion protein, a nucleic acid molecule encoding a variant CD80-Fc fusion protein is inserted into an appropriate expression vector. The resulting variant CD80-Fc fusion protein can be expressed in host cells transformed with the expression where assembly between Fc domains occurs by interchain disulfide bonds formed between the Fc moieties to yield dimeric, such as divalent, variant CD80-Fc fusion proteins.

The resulting Fc fusion proteins can be easily purified by affinity chromatography over Protein A or Protein G columns. For the generation of heterodimers, additional steps for purification can be necessary. For example, where two nucleic acids encoding different variant CD80 polypeptides are transformed into cells, the formation of heterodimers must be biochemically achieved since variant CD80 molecules carrying the Fc-domain will be expressed as disulfide-linked homodimers as well. Thus, homodimers can be reduced under conditions that favor the disruption of interchain disulfides, but do no effect intra-chain disulfides. In some cases, different variant-CD80 Fc monomers are mixed in equimolar amounts and oxidized to form a mixture of homo- and heterodimers. The components of this mixture are separated by chromatographic techniques. Alternatively, the formation of this type of heterodimer can be biased by genetically engineering and expressing Fc fusion molecules that contain a variant CD80 polypeptide using knob-into-hole methods described below.

C. Secreted Immunomodulatory Proteins (SIP) and Engineered Cells

Provided herein are engineered cells which express any of the immunomodulatory variant CD80 polypeptides (alternatively, “engineered cells). In some embodiments, the expressed immunomodulatory variant CD80 polypeptide is expressed and secreted from the cell (herein after also called a “secreted immunomodulatory protein” or SIP).

In some embodiments, the CD80 variant immunomodulatory polypeptide containing any one or more of the amino acid mutations as described herein, is secretable, such as when expressed from a cell. Such a variant CD80 immunomodulatory protein does not comprise a transmembrane domain. In some embodiments, the CD80 variant immunomodulatory protein that is secreted from the cell is a CD80-Fc fusion protein in which a variant CD80 polypeptide, such as any as described, is linked or fused, directly or indirectly, to an Fc region or domain. In some cases, the Fc region is inert and/or does not exhibit effector activity, such as any of the described Fc regions in which a wild-type Fc (e.g. IgG1) contains one or more amino acid mutations to reduce effector activity. In some cases, the Fc region is a wild-type Fc of an immunoglobulin (e.g. IgG1) and/or exhibits effector activity.

In particular embodiments, the variant CD80 immunomodulatory protein is a CD80 multivalent polypeptide, such as any as described or provided herein.

In some embodiments, the variant CD80 immunomodulatory protein comprises a signal peptide, e.g., an antibody signal peptide or other efficient signal sequence to get domains outside of cell. When the immunomodulatory protein comprises a signal peptide and is expressed by an engineered cell, the signal peptide causes the immunomodulatory protein to be secreted by the engineered cell. Generally, the signal peptide, or a portion of the signal peptide, is cleaved from the immunomodulatory protein with secretion. The immunomodulatory protein can be encoded by a nucleic acid (which can be part of an expression vector). In some embodiments, the immunomodulatory protein is expressed and secreted by a cell (such as an immune cell, for example a primary immune cell).

Thus, in some embodiments, there are provided variant CD80 immunomodulatory proteins that further comprises a signal peptide. In some embodiments, such a variant CD80 polypeptide is encoded by a nucleic acid molecule encoding an immunomodulatory protein under the operable control of a signal sequence for secretion. In some embodiments, the encoded immunomodulatory protein is secreted when expressed from a cell. In some embodiments, provided herein is a nucleic acid molecule encoding the variant CD80 immunomodulatory protein operably connected to a secretion sequence encoding the signal peptide.

A signal peptide is a sequence on the N-terminus of an immunomodulatory protein that signals secretion of the immunomodulatory protein from a cell. In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length).

In some embodiments, the signal peptide is a native signal peptide from the corresponding wild-type CD80 (see Table 1). In some embodiments, the signal peptide is a non-native signal peptide. For example, in some embodiments, the non-native signal peptide is a mutant native signal peptide from the corresponding wild-type CD80, and can include one or more (such as 2, 3, 4, 5, 6, 7, 8, 9, or 10 or more) substitutions insertions or deletions. In some embodiments, the non-native signal peptide is a signal peptide or mutant thereof of a family member from the same IgSF family as the wild-type IgSF family member. In some embodiments, the non-native signal peptide is a signal peptide or mutant thereof from an IgSF family member from a different IgSF family that the wild-type IgSF family member. In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2), or CD33), a serum albumin protein (e.g., HSA or albumin), a human azurocidin preprotein signal sequence, a luciferase, a trypsinogen (e.g., chymotrypsinogen or trypsinogen) or other signal peptide able to efficiently secrete a protein from a cell. Exemplary signal peptides, include any described in the Table 3.

TABLE 3
Exemplary Signal Peptides

| SEQ ID | Signal | |
|---|---|---|
| NO | Peptide | Peptide Sequence |
| SEQ ID | HSA | MKWVTFISLLFLFSSAYS |
| NO: 1547 | signal | |
| | peptide | |
| SEQ ID | Ig | MDMRAPAGIFGFLLVLFPG |
| NO: 1548 | kappa | YRS |
| | light | |
| | chain | |
| SEQ ID | human | MTRLTVLALLAGLLASSRA |
| NO: 1549 | azurocidin | |
| | preprotein | |
| | signal | |
| | sequence | |
| SEQ ID | IgG | MELGLSWIFLLAILKGVQC |
| NO: 1550 | heavy | |
| | chain | |
| | signal | |
| | peptide | |
| SEQ ID | IgG | MELGLRWVFLVAILEGVQC |
| NO: 1551 | heavy | |
| | chain | |
| | signal | |
| | peptide | |
| SEQ ID | IgG | MKHLWFFLLLVAAPRWVLS |
| NO: 1552 | heavy | |
| | chain | |
| | signal | |
| | peptide | |
| SEQ ID | IgG | MDWTWRILFLVAAATGAHS |
| NO: 1553 | heavy | |
| | chain | |
| | signal | |
| | peptide | |
| SEQ ID | IgG | MDWTWRFLFVVAAATGVQS |
| NO: 1554 | heavy | |
| | chain | |
| | signal | |
| | peptide | |
| SEQ ID | IgG | MEFGLSWLFLVAILKGVQC |
| NO: 1555 | heavy | |
| | chain | |
| | signal | |
| | peptide | |
| SEQ ID | IgG | MEFGLSWVFLVALFRGVQC |
| NO: 1556 | heavy | |
| | chain | |
| | signal | |
| | peptide | |
| SEQ ID | IgG | MDLLHKNMKHLWFFLLLVA |
| NO: 1557 | heavy | APRWVLS |
| | chain | |
| | signal | |
| | peptide | |
| SEQ ID | IgG | MDMRVPAQLLGLLLLWLS |
| NO: 1558 | Kappa | GARC |
| | light | |
| | chain | |
| | signal | |
| | sequences: | |
| SEQ ID | IgG | MKYLLPTAAAGLLLLAAQ |
| NO: 1559 | Kappa | PAMA |
| | light | |
| | chain | |
| | signal | |
| | sequences: | |
| SEQ ID | Gaussia | MGVKVLFALICIAVAEA |
| NO: 1560 | luciferase | |
| SEQ ID | Human | MKWVTFISLLFLFSSAYS |
| NO: 1561 | albumin | |
| SEQ ID | Human | MAFLWLLSCWALLGTTFG |
| NO: 1562 | chymotrypsinogen | |
| SEQ ID | Human | MQLLSCIALILALV |
| NO: 1563 | interleukin-2 | |
| SEQ ID | Human | MNLLLILTFVAAAVA |
| NO: 1564 | trypsinogen-2 | |
| SEQ ID | VH | MGSTAILALLLAVLQGVSA |
| NO: : 1546 | signal | |
| | peptide | |

In some embodiments of a secretable variant CD80 immunomodulatory protein, the immunomodulatory protein comprises a signal peptide when expressed, and the signal peptide (or a portion thereof) is cleaved from the immunomodulatory protein upon secretion.

1. Engineered Cells

Provided herein are engineered cells expressing any of the provided immunomodulatory polypeptide. In some embodiments, the engineered cells express and are capable of or are able to secrete the immunomodulatory protein from the cells under conditions suitable for secretion of the protein. In some embodiments, the immunomodulatory protein is expressed on a lymphocyte such as a tumor infiltrating lymphocyte (TIL), T-cell or NK cell, or on a myeloid cell. In some embodiments, the engineered cells are antigen presenting cells (APCs). In some embodiments, the engineered cells are engineered mammalian T-cells or engineered mammalian antigen presenting cells (APCs). In some embodiments, the engineered T-cells or APCs are human or murine cells.

In some embodiments, engineered T-cells include, but are not limited to, T helper cell, cytotoxic T-cell (alternatively, cytotoxic T lymphocyte or CTL), natural killer T-cell, regulatory T-cell, memory T-cell, or gamma delta T-cell. In some embodiments, the engineered T cells are CD4+ or CD8+. In addition to the signal of the MHC, engineered T-cells also require a co-stimulatory signal. Inn some embodiments, engineered T cells also can be modulated by inhibitory signals, which, in some cases, is provided by a variant CD80 transmembrane immunomodulatory polypeptide expressed in membrane bound form as discussed previously.

In some embodiments, the engineered APCs include, for example, MHC II expressing APCs such as macrophages, B cells, and dendritic cells, as well as artificial APCs (aAPCs) including both cellular and acellular (e.g., biodegradable polymeric microparticles) aAPCs. Artificial APCs (aAPCs) are synthetic versions of APCs that can act in a similar manner to APCs in that they present antigens to T-cells as well as activate them. Antigen presentation is performed by the MHC (Class I or Class II). In some embodiments, in engineered APCs such as aAPCs, the antigen that is loaded onto the MHC is, in some embodiments, a tumor specific antigen or a tumor associated antigen. The antigen loaded onto the MHC is recognized by a T-cell receptor (TCR) of a T cell, which, in some cases, can express CTLA-4, CD28, PD-L1 or other molecules recognized by the variant CD80 polypeptides provided herein. Materials which can be used to engineer an aAPC include: poly (glycolic acid), poly(lactic-co-glycolic acid), iron-oxide, liposomes, lipid bilayers, sepharose, and polystyrene.

In some embodiments a cellular aAPC can be engineered to contain a secreted CD80 immunomodulatory polypeptide or SIP and TCR agonist which is used in adoptive cellular therapy. In some embodiments, a cellular aAPC can be engineered to contain a SIP and TCR agonist which is used in ex vivo expansion of human T cells, such as prior to administration, e.g., for reintroduction into the patient. In some aspects, the aAPC may include expression of at least one anti-CD3 antibody clone, e.g., such as, for example, OKT3 and/or UCHT1. In some aspects, the aAPCs may be inactivated (e.g., irradiated).

In some embodiments, an immunomodulatory protein provided herein, such as a secretable immunomodulatory protein, is co-expressed or engineered into a cell that expresses an antigen-binding receptor, such as a recombinant receptor, such as a chimeric antigen receptor (CAR) or T cell receptor (TCR). In some embodiments, the engineered cell, such as an engineered T cell, recognizes a desired antigen associated with cancer, inflammatory and autoimmune disorders, or a viral infection. In specific embodiments, the antigen-binding receptor contains an antigen-binding moiety that specifically binds a tumor specific antigen or a tumor associated antigen. In some embodiments, the engineered T-cell is a CAR (chimeric antigen receptor) T-cell that contains an antigen-binding domain (e.g., scFv) that specifically binds to an antigen, such as a tumor specific antigen or tumor associated antigen. In some embodiments, the secreted CD80 immunomodulatory protein or sIP protein is expressed by an engineered T-cell receptor cell or an engineered chimeric antigen receptor cell. In such embodiments, the engineered cell co-expresses the SIP and the CAR or TCR, and secretes the SIP from the cell.

Chimeric antigen receptors (CARs) are recombinant receptors that include an antigen-binding domain (ectodomain), a transmembrane domain and an intracellular signaling region (endodomain) that is capable of inducing or mediating an activation signal to the T cell after the antigen is bound. In some example, CAR-expressing cells are engineered to express an extracellular single chain variable fragment (scFv) with specificity for a particular tumor antigen linked to an intracellular signaling part comprising an activating domain and, in some cases, a costimulatory domain. The costimulatory domain can be derived from, e.g., CD28, OX-40, 4-1BB/CD137, inducible T cell costimulator (ICOS), The activating domain can be derived from, e.g., CD3, such as CD3 zeta, epsilon, delta, gamma, or the like. In certain embodiments, the CAR is designed to have two, three, four, or more costimulatory domains. The CAR scFv can be designed to target an antigen expressed on a cell associated with a disease or condition, e.g., a tumor antigen, such as, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in U.S. Patent Publication Nos. 2013/0287748, 2014/0227237, 2014/0099309, and 2014/0050708, and these references are incorporated by reference in their entirety.

In some aspects, the antigen-binding domain is an antibody or antigen-binding fragment thereof, such as a single chain fragment (scFv). In some embodiments, the antigen is expressed on a tumor or cancer cell. Exemplary of an antigen is CD19. Exemplary of a CAR is an anti-CD19 CAR, such as a CAR containing an anti-CD19 scFv set forth in SEQ ID NO: 1565. In some embodiments, the CAR further contains a spacer, a transmembrane domain, and an intracellular signaling domain or region comprising an ITAM signaling domain, such as a CD3zeta signaling domain. In some embodiments, the CAR further includes a costimulatory signaling domain. In some embodiments, the spacer and transmembrane domain are the hinge and transmembrane domain derived from CD8, such as having an exemplary sequence set forth in SEQ ID NO: 1566, 1567, or 1568 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:332, 364, 1997. In some embodiments, the endodomain comprises at CD3-zeta signaling domain. In some embodiments, the CD3-zeta signaling domain comprises the sequence of amino acids set forth in SEQ ID NO: 1569 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO: 1569 and retains the activity of T cell signaling. In some embodiments, the endodomain of a CAR can further comprise a costimulatory signaling domain or region to further modulate immunomodulatory responses of the T-cell. In some embodiments, the costimulatory signaling domain is or comprises a costimulatory region, or is derived from a costimulatory region, of CD28, ICOS, 41BB or OX40. In some embodiments, the costimulatory signaling domain is a derived from CD28 or 4-1BB and comprises the sequence of amino acids set forth in any of SEQ ID NOS: 1570-1573 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO: 1570-1573 and retains the activity of T cell costimulatory signaling.

In some embodiments, the construct encoding the CAR further encodes a second protein, such as a marker, e.g., detectable protein, separated from the CAR by a self-cleaving peptide sequence. In some embodiments, the self-cleaving peptide sequence is an F2A, T2A, E2A or P2A self-cleaving peptide. Exemplary sequences of a T2A self-cleaving peptide are set for the in any one of SEQ ID NOS: 1574, 1575, or 1576 or a sequence of amino acids that exhibits at least 85%, 86%, 87%, 88%, 89%, 90%, 91%, 92%, 93%, 94%, 95%, 96%, 97%, 98% or 99% or more sequence identity to any of SEQ ID NOS: 1574, 1575, or 1576. In some embodiments, the T2A is encoded by the sequence of nucleotides set forth in SEQ ID NO: 1576 or a sequence that exhibits at least 85%, 86%, 87%, 88%, 89%, 90%, 91%, 92%, 93%, 94%, 95%, 96%, 97%, 98% or 99% or more sequence identity to any of SEQ ID NO: 2008. An exemplary sequence of a P2A self-cleaving peptide is set in SEQ ID NO: 1577 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NOS: 1577. In some cases, a nucleic acid construct that encodes more than one P2A self-cleaving peptide (such as a P2A1 and P2A2), in which the nucleotide sequence P2A1 and P2A2 each encode the P2A set forth in SEQ ID NO: 1577, the nucleotide sequence may be different to avoid recombination between sequences.

In some embodiments, the marker is a detectable protein, such as a fluorescent protein, e.g., a green fluorescent protein (GFP) or blue fluorescent protein (BFP). Exemplary sequences of a fluorescent protein marker are set forth in SEQ ID NO: 1578-1582, or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO: 1578-1582.

In some embodiments, the CAR has the sequence of amino acids set forth in any of SEQ ID NOS: 1583-1590 or a sequence of amino acids that exhibits at least 85%, 86%, 87%, 88%, 89%, 90%, 91%, 92%, 93%, 94%, 95%, 96%, 97%, 98% or 99% or more sequence identity to any one of SEQ ID NOS: 1583-1590. In some embodiments, the CAR is encoded by a sequence of nucleotides set forth in SEQ ID NO: 1591 or 1592 or a sequence of amino acids that exhibits at least 85%, 86%, 87%, 88%, 89%, 90%, 91%, 92%, 93%, 94%, 95%, 96%, 97%, 98% or 99% or more sequence identity to any one of SEQ ID NO: 1591 or 1592.

In another embodiment, the engineered T-cell possesses a TCR, including a recombinant or engineered TCR. In some embodiments, the TCR can be a native TCR. Those of skill in the art will recognize that generally native mammalian T-cell receptors comprise an alpha and a beta chain (or a gamma and a delta chain) involved in antigen specific recognition and binding. In some embodiments, the TCR is an engineered TCR that is modified. In some embodiments, the TCR of an engineered T-cell specifically binds to a tumor associated or tumor specific antigen presented by an APC. In some embodiments, the TCR is a TCR specific to HPV E6, such as described in WO 2015/009606. In some embodiments, the TCRα and TCRβ chain sequences can be constructed as part of the same expression vector in which the encoding nucleic acids are separated from each other by a sequence encoding a self-cleaving peptide, such as a P2A or T2A ribosome skip sequence.

In some embodiments, the immunomodulatory polypeptides, such as secretable immunomodulatory polypeptides, can be incorporated into engineered cells, such as engineered T cells or engineered APCs, by a variety of strategies such as those employed for recombinant host cells. A variety of methods to introduce a DNA construct into primary T cells are known in the art. In some embodiments, viral transduction or plasmid electroporation are employed. In typical embodiments, the nucleic acid molecule encoding the immunomodulatory protein, or the expression vector, comprises a signal peptide that localizes the expressed immunomodulatory proteins for secretion. In some embodiments, a nucleic acid encoding a secretable immunomodulatory protein of the invention is sub-cloned into a viral vector, such as a retroviral vector, which allows expression in the host mammalian cell. The expression vector can be introduced into a mammalian host cell and, under host cell culture conditions, the immunomodulatory protein is secreted.

In an exemplary example, primary T-cells can be purified ex vivo (CD4 cells or CD8 cells or both) and stimulated with an activation protocol consisting of various TCR/CD28 agonists, such as anti-CD3/anti-CD28 coated beads. After a 2 or 3 day activation process, a recombinant expression vector containing an immunomodulatory polypeptide can be stably introduced into the primary T cells through art standard lentiviral or retroviral transduction protocols or plasmid electroporation strategies. Cells can be monitored for immunomodulatory polypeptide expression by, for example, flow cytometry using anti-epitope tag or antibodies that cross-react with native parental molecule and polypeptides comprising variant CD80. T-cells that express the immunomodulatory polypeptide can be enriched through sorting with anti-epitope tag antibodies or enriched for high or low expression depending on the application.

Upon immunomodulatory polypeptide expression the engineered T-cell can be assayed for appropriate function by a variety of means. The engineered CAR or TCR co-expression can be validated to show that this part of the engineered T cell was not significantly impacted by the expression of the immunomodulatory protein. Once validated, standard in vitro cytotoxicity, proliferation, or cytokine assays (e.g., IFN-gamma expression) can be used to assess the function of engineered T-cells. Exemplary standard endpoints are percent lysis of the tumor line, proliferation of the engineered T-cell, or IFN-gamma protein expression in culture supernatants. An engineered construct which results in statistically significant increased lysis of tumor line, increased proliferation of the engineered T-cell, or increased IFN-gamma expression over the control construct can be selected for. Additionally, non-engineered, such as native primary or endogenous T-cells could also be incorporated into the same in vitro assay to measure the ability of the immunomodulatory polypeptide construct expressed on the engineered cells, such as engineered T-cells, to modulate activity, including, in some cases, to activate and generate effector function in bystander, native T-cells. Increased expression of activation markers such as CD69, CD44, or CD62L could be monitored on endogenous T cells, and increased proliferation and/or cytokine production could indicate desired activity of the immunomodulatory protein expressed by the engineered T cells.

In some embodiments, the similar assays can be used to compare the function of engineered T cells containing the CAR or TCR alone to those containing the CAR or TCR and a SIP construct. Typically, these in vitro assays are performed by plating various ratios of the engineered T cell and a “tumor” cell line containing the cognate CAR or TCR antigen together in culture. Standard endpoints are percent lysis of the tumor line, proliferation of the engineered T cell, or IFN-gamma production in culture supernatants. An engineered immunomodulatory protein which resulted in statistically significant increased lysis of tumor line, increased proliferation of the engineered T cell, or increased IFN-gamma production over the same TCR or CAR construct alone can be selected for. Engineered human T cells can be analyzed in immunocompromised mice, like the NSG strain, which lacks mouse T, NK and B cells. Engineered human T cells in which the CAR or TCR binds a target counter-structure on the xenograft and is co-expressed with the SIP affinity modified IgSF domain can be adoptively transferred in vivo at different cell numbers and ratios compared to the xenograft. For example, engraftment of CD19+ leukemia tumor lines containing a luciferase/GFP vector can be monitored through bioluminescence or ex vivo by flow cytometry. In a common embodiment, the xenograft is introduced into the murine model, followed by the engineered T cells several days later. Engineered T cells containing the immunomodulatory protein can be assayed for increased survival, tumor clearance, or expanded engineered T cells numbers relative to engineered T cells containing the CAR or TCR alone. As in the in vitro assay, endogenous, native (i.e., non-engineered) human T cells could be co-adoptively transferred to look for successful epitope spreading in that population, resulting in better survival or tumor clearance.

D. Nucleic Acids, Vectors and Methods for Producing the Polypeptides or Cells

Provided herein are isolated or recombinant nucleic acids collectively referred to as “nucleic acids” which encode any of the various provided embodiments of the variant CD80 polypeptides or variant CD80 IgSF domain fusion proteins provided herein. In some embodiments, nucleic acids provided herein, including all described below, are useful in recombinant production (e.g., expression) of variant CD80 polypeptides or variant CD80 IgSF domain fusion proteins provided herein. The nucleic acids provided herein can be in the form of RNA or in the form of DNA, and include mRNA, cRNA, recombinant or synthetic RNA and DNA, and cDNA. The nucleic acids provided herein are typically DNA molecules, and usually double-stranded DNA molecules. However, single-stranded DNA, single-stranded RNA, double-stranded RNA, and hybrid DNA/RNA nucleic acids or combinations thereof comprising any of the nucleotide sequences of the invention also are provided.

Also provided herein are recombinant expression vectors and recombinant host cells useful in producing the variant CD80 polypeptides or variant CD80 IgSF domain fusion proteins provided herein.

In any of the above provided embodiments, the nucleic acids encoding the variant CD80 IgSF domain fusion proteins provided herein can be introduced into cells using recombinant DNA and cloning techniques. To do so, a recombinant DNA molecule encoding an immunomodulatory polypeptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidite method. Also, a combination of these techniques could be used. In some instances, a recombinant or synthetic nucleic acid may be generated through polymerase chain reaction (PCR). In some embodiments, a DNA insert can be generated encoding one or more variant CD80 polypeptides containing at least one affinity-modified IgSF domain and, in some embodiments, a multimerization domain (e.g. Fc domain) in accord with the provided description. This DNA insert can be cloned into an appropriate transduction/transfection vector as is known to those of skill in the art. Also provided are expression vectors containing the nucleic acid molecules.

In some embodiments, the expression vectors are capable of expressing the variant CD80 IgSF domain fusion proteins in an appropriate cell under conditions suited to expression of the protein. In some aspects, nucleic acid molecule or an expression vector comprises the DNA molecule that encodes the immunomodulatory protein operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

In some embodiments, expression of the variant CD80 IgSF domain fusion protein is controlled by a promoter or enhancer to control or regulate expression. The promoter is operably linked to the portion of the nucleic acid molecule encoding the variant polypeptide or immunomodulatory protein. In some embodiments, the promotor is a constitutively active promotor (such as a tissue-specific constitutively active promotor or other constitutive promotor). In some embodiments, the promotor is an inducible promotor, which may be responsive to an inducing agent (such as a T cell activation signal).

In some embodiments, a constitutive promoter is operatively linked to the nucleic acid molecule encoding the variant polypeptide or immunomodulatory protein. Exemplary constitutive promoters include the Simian vacuolating virus 40 (SV40) promoter, the cytomegalovirus (CMV) promoter, the ubiquitin C (UbC) promoter, and the EF-1 alpha (EF1a) promoter. In some embodiments, the constitutive promoter is tissue specific. For example, in some embodiments, the promoter allows for constitutive expression of the immunomodulatory protein in specific tissues, such as immune cells, lymphocytes, or T cells. Exemplary tissue-specific promoters are described in U.S. Pat. No. 5,998,205, including, for example, a fetoprotein, DF3, tyrosinase, CEA, surfactant protein, and ErbB2 promoters.

In some embodiments, an inducible promoter is operatively linked to the nucleic acid molecule encoding the variant polypeptide or immunomodulatory protein such that expression of the nucleic acid is controllable by controlling the presence or absence of the appropriate inducer of transcription. For example, the promoter can be a regulated promoter and transcription factor expression system, such as the published tetracycline-regulated systems or other regulatable systems (see, e.g., published International PCT Appl. No. WO 01/30843), to allow regulated expression of the encoded polypeptide. An exemplary regulatable promoter system is the Tet-On (and Tet-Off) system available, for example, from Clontech (Palo Alto, Calif.). This promoter system allows the regulated expression of the transgene controlled by tetracycline or tetracycline derivatives, such as doxycycline. Other regulatable promoter systems are known (see e.g., published U.S. Application No. 2002-0168714, entitled “Regulation of Gene Expression Using Single-Chain, Monomeric, Ligand Dependent Polypeptide Switches,” which describes gene switches that contain ligand binding domains and transcriptional regulating domains, such as those from hormone receptors).

In some embodiments, the promotor is responsive to an element responsive to T-cell activation signaling. Solely by way of example, in some embodiments, an engineered T cell comprises an expression vector encoding the immunomodulatory protein and a promotor operatively linked to control expression of the immunomodulatory protein. The engineered T cell can be activated, for example by signaling through an engineered T cell receptor (TCR) or a chimeric antigen rector (CAR), and thereby triggering expression and secretion of the immunomodulatory protein through the responsive promotor.

In some embodiments, an inducible promoter is operatively linked to the nucleic acid molecule encoding the immunomodulatory protein such that the immunomodulatory protein is expressed in response to a nuclear factor of activated T-cells (NFAT) or nuclear factor kappa-light-chain enhancer of activated B cells (NF-κB). For example, in some embodiments, the inducible promoter comprises a binding site for NFAT or NF-κB. For example, in some embodiments, the promoter is an NFAT or NF-κB promoter or a functional variant thereof. Thus, in some embodiments, the nucleic acids make it possible to control the expression of immunomodulatory protein while also reducing or eliminating the toxicity of the immunomodulatory protein. In particular, engineered immune cells comprising the nucleic acids of the invention express and secrete the immunomodulatory protein only when the cell (e.g., a T-cell receptor (TCR) or a chimeric antigen receptor (CAR) expressed by the cell) is specifically stimulated by an antigen and/or the cell (e.g., the calcium signaling pathway of the cell) is non-specifically stimulated by, e.g., phorbol myristate acetate (PMA)/Ionomycin. Accordingly, the expression and, in some cases, secretion, of immunomodulatory protein can be controlled to occur only when and where it is needed (e.g., in the presence of an infectious disease-causing agent, cancer, or at a tumor site), which can decrease or avoid undesired immunomodulatory protein interactions.

In some embodiments, the nucleic acid encoding a variant CD80 IgSF domain fusion protein described herein comprises a suitable nucleotide sequence that encodes a NFAT promoter, NF-κB promoter, or a functional variant thereof. “NFAT promoter” as used herein means one or more NFAT responsive elements linked to a minimal promoter. “NF-κB promoter” refers to one or more NF-κB responsive elements linked to a minimal promoter. In some embodiments, the minimal promoter of a gene is a minimal human IL-2 promoter or a CMV promoter. The NFAT responsive elements may comprise, e.g., NFAT1, NFAT2, NFAT3, and/or NFAT4 responsive elements. The NFAT promoter, NF-κB promoter, or a functional variant thereof may comprise any number of binding motifs, e.g., at least two, at least three, at least four, at least five, or at least six, at least seven, at least eight, at least nine, at least ten, at least eleven, or up to twelve binding motifs.

The resulting recombinant expression vector having the DNA molecule thereon is used to transform an appropriate host. This transformation can be performed using methods well known in the art. In some embodiments, a nucleic acid provided herein further comprises nucleotide sequence that encodes a secretory or signal peptide operably linked to the nucleic acid encoding an immunomodulatory polypeptide such that a resultant soluble immunomodulatory polypeptide is recovered from the culture medium, host cell, or host cell periplasm. In other embodiments, the appropriate expression control signals are chosen to allow for membrane expression of an immunomodulatory polypeptide. Furthermore, commercially available kits as well as contract manufacturing companies can also be utilized to make engineered cells or recombinant host cells provided herein.

In some embodiments, the resulting expression vector having the DNA molecule thereon is used to transform, such as transduce, an appropriate cell. The introduction can be performed using methods well known in the art. Exemplary methods include those for transfer of nucleic acids encoding the receptors, including via viral, e.g., retroviral or lentiviral, transduction, transposons, and electroporation. In some embodiments, the expression vector is a viral vector. In some embodiments, the nucleic acid is transferred into cells by lentiviral or retroviral transduction methods.

Any of a large number of publicly available and well-known mammalian host cells, including mammalian T-cells or APCs, can be used in the preparing the polypeptides or engineered cells. The selection of a cell is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all cells can be equally effective for the expression of a particular DNA sequence.

In some embodiments, the host cells can be a variety of eukaryotic cells, such as in yeast cells, or with mammalian cells such as Chinese hamster ovary (CHO) or HEK293 cells. In some embodiments, the host cell is a suspension cell and the polypeptide is engineered or produced in cultured suspension, such as in cultured suspension CHO cells, e.g., CHO-S cells. In some examples, the cell line is a CHO cell line that is deficient in DHFR (DHFR−), such as DG44 and DUXB11. In some embodiments, the cell is deficient in glutamine synthase (GS), e.g., CHO-S cells, CHOK1 SV cells, and CHOZN((R)) GS−/− cells. In some embodiments, the CHO cells, such as suspension CHO cells, may be CHO-S-2H2 cells, CHO-S-clone 14 cells, or ExpiCHO-S cells.

In some embodiments, host cells can also be prokaryotic cells, such as with E. coli. The transformed recombinant host is cultured under polypeptide expressing conditions, and then purified to obtain a soluble protein. Recombinant host cells can be cultured under conventional fermentation conditions so that the desired polypeptides are expressed. Such fermentation conditions are well known in the art. Finally, the polypeptides provided herein can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, and affinity chromatography. Protein refolding steps can be used, as desired, in completing configuration of the mature protein. Finally, high performance liquid chromatography (HPLC) can be employed in the final purification steps.

In some embodiments, the cell is an immune cell, such as any described above in connection with preparing engineered cells. In some embodiments, such engineered cells are primary cells. In some embodiments, the engineered cells are autologous to the subject. In some embodiment, the engineered cells are allogeneic to the subject. In some embodiments, the engineered cells are obtained from a subject, such as by leukapheresis, and transformed ex vivo for expression of the immunomodulatory polypeptide, e.g., transmembrane immunomodulatory polypeptide or secretable immunomodulatory polypeptide.

In some embodiments, the recombinant vector is a plasmid or cosmid. Plasmid or cosmid containing nucleic acid sequences encoding the variant immunomodulatory polypeptides, as described herein, is readily constructed using standard techniques well known in the art. For generation of the infectious agent, the vector or genome can be constructed in a plasmid form that can then be transfected into a packaging or producer cell line or a host bacterium. The recombinant vectors can be generated using any of the recombinant techniques known in the art. In some embodiments, the vectors can include a prokaryotic origin of replication and/or a gene whose expression confers a detectable or selectable marker such as a drug resistance for propagation and/or selection in prokaryotic systems.

In some embodiments, the recombinant vector is a viral vector. Exemplary recombinant viral vectors include a lentiviral vector genome, poxvirus vector genome, vaccinia virus vector genome, adenovirus vector genome, adenovirus-associated virus vector genome, herpes virus vector genome, and alpha virus vector genome. Viral vectors can be live, attenuated, replication conditional or replication deficient, non-pathogenic (defective), replication competent viral vector, and/or is modified to express a heterologous gene product, e.g., the variant immunomodulatory polypeptides provided herein. Vectors for generation of viruses also can be modified to alter attenuation of the virus, which includes any method of increasing or decreasing the transcriptional or translational load.

Exemplary viral vectors that can be used include modified vaccinia virus vectors (see, e.g., Guerra et al., J. Virol. 80:985-98 (2006); Tartaglia et al., AIDS Research and Human Retroviruses 8: 144547 (1992); Gheradi et al., J. Gen. Virol. 86:2925-36 (2005); Mayr et al., Infection 3:6-14 (1975); Hu et al., J. Virol. 75: 10300-308 (2001); U.S. Pat. Nos. 5,698,530, 6,998,252, 5,443,964, 7,247,615 and 7,368,116); adenovirus vector or adenovirus-associated virus vectors (see, e.g., Molin et al., J. Virol. 72:8358-61 (1998); Narumi et al., Am J. Respir. Cell Mol. Biol. 19:93641 (1998); Mercier et al., Proc. Natl. Acad. Sci. USA 101:6188-93 (2004); U.S. Pat. Nos. 6,143,290; 6,596,535; 6,855,317; 6,936,257; 7,125,717; 7,378,087; 7,550,296); retroviral vectors including those based upon murine leukemia virus (MuLV), gibbon ape leukemia virus (GaLV), ecotropic retroviruses, simian immunodeficiency virus (SIV), human immunodeficiency virus (HIV), and combinations (see, e.g., Buchscher et al., J. Virol. 66:2731-39 (1992); Johann et al., J. Virol. 66: 1635-40 (1992); Sommerfelt et al., Virology 176:58-59 (1990); Wilson et al., J. Virol. 63:2374-78 (1989); Miller et al., J. Virol. 65:2220-24 (1991); Miller et al., Mol. Cell Biol. 10:4239 (1990); Kolberg, NIH Res. 4:43 1992; Cornetta et al., Hum. Gene Ther. 2:215 (1991)); lentiviral vectors including those based upon Human Immunodeficiency Virus (HIV-1), HIV-2, feline immunodeficiency virus (FIV), equine infectious anemia virus, Simian Immunodeficiency Virus (SIV), and maedi/visna virus (see, e.g., Pfeifer et al., Annu. Rev. Genomics Hum. Genet. 2: 177-211 (2001); Zufferey et al., J. Virol. 72: 9873, 1998; Miyoshi et al., J. Virol. 72:8150, 1998; Philpott and Thrasher, Human Gene Therapy 18:483, 2007; Engelman et al., J. Virol. 69: 2729, 1995; Nightingale et al., Mol. Therapy, 13: 1121, 2006; Brown et al., J. Virol. 73:9011 (1999); WO 2009/076524; WO 2012/141984; WO 2016/011083; McWilliams et al., J. Virol. 77: 11150, 2003; Powell et al., J. Virol. 70:5288, 1996) or any, variants thereof, and/or vectors that can be used to generate any of the viruses described above. In some embodiments, the recombinant vector can include regulatory sequences, such as promoter or enhancer sequences, that can regulate the expression of the viral genome, such as in the case for RNA viruses, in the packaging cell line (see, e.g., U.S. Pat. Nos. 5,385,839 and 5,168,062).

In some aspects, nucleic acids or an expression vector comprises a nucleic acid sequence that encodes the immunomodulatory protein operatively linked to appropriate expression control sequences. Methods of affecting this operative linking, either before or after the nucleic acid sequence encoding the immunomodulatory protein is inserted into the vector, are well known. Expression control sequences include promoters, activators, enhancers, operators, ribosomal binding sites, start signals, stop signals, cap signals, polyadenylation signals, and other signals involved with the control of transcription or translation. The promoter can be operably linked to the portion of the nucleic acid sequence encoding the immunomodulatory protein. In some embodiments, the promotor is a constitutively active promotor in the target cell (such as a tissue-specific constitutively active promotor or other constitutive promotor). For example, the recombinant expression vector may also include, lymphoid tissue-specific transcriptional regulatory elements (TRE) such as a B lymphocyte, T lymphocyte, or dendritic cell specific TRE. Lymphoid tissue specific TRE are known in the art (see, e.g., Thompson et al., Mol. Cell. Biol. 12:1043-53 (1992); Todd et al., J. Exp. Med. 177:1663-74 (1993); Penix et al., J. Exp. Med. 178:1483-96 (1993)). In some embodiments, the promotor is an inducible promotor, which may be responsive to an inducing agent (such as a T cell activation signal). In some embodiments, nucleic acids delivered to the target cell in the subject, e.g., tumor cell, immune cell and/or APC, can be operably linked to any of the regulatory elements described above.

In some embodiments, the vector is a bacterial vector, e.g., a bacterial plasmid or cosmid. In some embodiments, the bacterial vector is delivered to the target cell, e.g., tumor cells, immune cells and/or APCs, via bacterial-mediated transfer of plasmid DNA to mammalian cells (also referred to as “bactofection”). In some embodiments, the delivered bacterial vector also contains appropriate expression control sequences for expression in the target cells, such as a promoter sequence and/or enhancer sequences, or any regulatory or control sequences described above. In some embodiments, the bacterial vector contains appropriate expression control sequences for expression and/or secretion of the encoded variant polypeptides in the infectious agent, e.g., the bacterium.

In some embodiments, polypeptides provided herein can also be made by synthetic methods. Solid phase synthesis is the preferred technique of making individual peptides since it is the most cost-effective method of making small peptides. For example, well known solid phase synthesis techniques include the use of protecting groups, linkers, and solid phase supports, as well as specific protection and deprotection reaction conditions, linker cleavage conditions, use of scavengers, and other aspects of solid phase peptide synthesis. Peptides can then be assembled into the polypeptides as provided herein.

### II. METHODS OF ASSESSING ACTIVITY IMMUNE MODULATION OF VARIANT CD80 IGSF DOMAIN FUSION PROTEINS

In some embodiments, the variant CD80 IgSF domain fusion proteins provided herein exhibit immunomodulatory activity to modulate T cell activation. In some embodiments, the variant CD80 IgSF domain fusion proteins modulate IFN-gamma expression in a T cell assay relative to a wild-type or unmodified CD80 control. In some cases, modulation of IFN-gamma expression can increase IFN-gamma expression relative to the control. Assays to determine specific binding and IFN-gamma expression are well-known in the art and include the MLR (mixed lymphocyte reaction) assays measuring interferon-gamma cytokine levels in culture supernatants (Wang et al., Cancer Immunol Res. 2014 September: 2(9):846-56), SEB (staphylococcal enterotoxin B) T cell stimulation assay (Wang et al., Cancer Immunol Res. 2014 September: 2(9):846-56), and anti-CD3 T cell stimulation assays (Li and Kurlander, J Transl Med. 2010: 8: 104).

In some embodiments, a variant CD80 IgSF domain fusion protein can in some embodiments, alter (e.g. increase) IFN-gamma (interferon-gamma) expression in a primary T-cell assay relative to a wild-type CD80 control. In some embodiments, a variant CD80 polypeptide or variant CD80 IgSF domain fusion protein is an antagonist of the inhibitory receptor, such as blocks an inhibitory signal in the cell that may occur to decrease response to an activating stimulus, e.g., CD3 and/or CD28 costimulatory signal or a mitogenic signal. Those of skill will recognize that different formats of the primary T-cell assay used to determine an increase or decrease in IFN-gamma expression exist.

In assaying for the ability of a variant CD80 to increase IFN-gamma expression in a primary T-cell assay, a Mixed Lymphocyte Reaction (MLR) assay can be used. In some embodiments, a variant CD80 polypeptide or variant CD80 IgSF domain fusion protein blocks activity of the CTLA-4 inhibitory receptor or PD-L1 and thereby increase MLR activity in the assay, such as observed by increased production of IFN-gamma in the assay. In some embodiments, a variant CD80 polypeptide or immunomodulatory protein exhibits agonist activity, and/or may block activity of the CTLA-4 inhibitory receptor and thereby increase MLR activity, such as increase IFN-gamma production.

Alternatively, in assaying for the ability of a variant CD80 to modulate or increase IFN-gamma expression in a primary T-cell assay, a co-immobilization assay can be used. In a co-immobilization assay, a TCR signal, provided in some embodiments by anti-CD3 antibody, is used in conjunction with a co-immobilized variant CD80 to determine the ability to increase or decrease IFN-gamma expression relative to a CD80 unmodified or wild-type control. In some embodiments, a variant CD80 polypeptide or variant CD80 IgSF domain fusion protein, e.g., CD80-Fc, increases IFN-gamma production in a co-immobilization assay.

In some embodiments, in assaying for the ability of a variant CD80 to increase IFN-gamma expression a T cell reporter assay can be used. In some embodiments, the T cell is a Jurkat T cell line or is derived from Jurkat T cell lines. In reporter assays, the reporter cell line (e.g., Jurkat reporter cell) also is generated to overexpress an inhibitory receptor that is the cognate binding partner of the variant IgSF domain polypeptide. For example, in the case of a variant CD80, the reporter cell line (e.g., Jurkat reporter cell) is generated to overexpress CTLA-4. In other examples, the reporter cell line (e.g., Jurkat reporter cell) is generated to overexpress PD-L1. In some embodiments, the reporter T cells also contain a reporter construct containing an inducible promoter responsive to T cell activation operably linked to a reporter. In some embodiments, the reporter is a fluorescent or luminescent reporter. In some embodiments, the reporter is luciferase. In some embodiments, the promoter is responsive to CD3 signaling. In some embodiments, the promoter is an NFAT promoter. In some embodiments, the promoter is responsive to costimulatory signaling, e.g., CD28 costimulatory signaling. In some embodiments, the promoter is an IL-2 promoter.

In aspects of a reporter assay, a reporter cell line is stimulated, such as by co-incubation with antigen presenting cells (APCs) expressing the wild-type ligand of the inhibitory receptor, e.g., CD80. In some embodiments, the APCs are artificial APCs. Artificial APCs are well known to a skilled artisan. In some embodiments, artificial APCs are derived from one or more mammalian cell line, such as K562, CHO or 293 cells. In some embodiments, the artificial APCs are engineered to express an anti-CD3 antibody and, in some cases, a costimulatory ligand. In some embodiments, the artificial APC is generated to overexpress the cognate binding partner of the variant IgSF domain polypeptide. For example, in the case of a variant CD80, the reporter cell line (e.g., Jurkat reporter cell) is generated to overexpress the inhibitory ligand PD-L1.

In some embodiments, the Jurkat reporter cells are co-incubated with artificial APCs overexpressing the inhibitory ligand in the presence of the variant IgSF domain molecule or immunomodulatory protein, e.g., variant CD80 polypeptide or variant CD80 IgSF domain fusion protein. In some embodiments, reporter expression is monitored, such as by determining the luminescence or fluorescence of the cells. In some embodiments, normal interactions between its inhibitory receptor and ligand result in a repression of or decrease in the reporter signal, such as compared to control, e.g., reporter expression by co-incubation of control T cells and APCs in which the inhibitory receptor and ligand interaction is not present, e.g., APCs that do not overexpress CD80. In certain embodiments provided herein, a variant CD80 polypeptide or immunomodulatory protein mediates CD28 agonism, such as such as PD-L1-dependent CD28 costimulation, e.g. when provided in soluble form as a variant CD80-Fc, thereby resulting in an increase of the reporter signal compared to the absence of the variant CD80 polypeptide or immunomodulatory protein. In some cases, certain formats of a variant CD80 polypeptide or immunomodulatory protein as provided herein may provide a blocking activity of an inhibitory receptor, thereby increasing reporter expression compared to the absence of the variant CD80 polypeptide or immunomodulatory protein.

Use of proper controls is known to those of skill in the art, however, in the aforementioned embodiments, a control typically involves use of the unmodified CD80, such as a wild-type of native CD80 isoform from the same mammalian species from which the variant CD80 was derived or developed. In some embodiments, the wild-type or native CD80 is of the same form or corresponding form as the variant. For example, if the variant CD80 is a soluble form containing a variant ECD fused to an Fc protein, then the control is a soluble form containing the wild-type or native ECD of CD80 fused to the Fc protein. Irrespective of whether the binding affinity to either one or more of CD28, CTLA-4 and PD-L1 is increased or decreased, a variant CD80 in some embodiments will increase IFN-gamma expression in a T-cell assay relative to a wild-type CD80 control.

In some embodiments, a variant CD80 polypeptide or immunomodulatory protein, increases IFN-gamma expression (i.e., protein expression) relative to a wild-type or unmodified CD80 control by at least: 5%, 10%, 20%, 30%, 40%, 50%, 60%, 70%, 80%, 90%, or higher. In some embodiments, the wild-type CD80 control is murine CD80, such as would typically be used for a variant CD80 altered in sequence from that of a wild-type murine CD80 sequence. In some embodiments, the wild-type CD80 control is human CD80, such as would typically be used for a variant CD80 altered in sequence from that of a corresponding wild-type human CD80 sequence such as an CD80 sequence comprising the sequence of amino acids of SEQ ID NO: 1, SEQ ID NO: 2 or SEQ ID NO: 76 or SEQ ID NO:150 or SEQ ID NO: 1245.

### III. PHARMACEUTICAL FORMULATIONS, ADMINISTRATION, AND ARTICLES OF MANUFACTURE OR KITS

Provided herein are compositions containing any of the variant CD80 polypeptides or variant CD80 IgSF domain fusion proteins described herein. The pharmaceutical composition can further comprise a pharmaceutically acceptable excipient. For example, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

In some embodiments, the pharmaceutical composition is a solid, such as a powder, capsule, or tablet. For example, the components of the pharmaceutical composition can be lyophilized. In some embodiments, the solid pharmaceutical composition is reconstituted or dissolved in a liquid prior to administration.

In some embodiments, the pharmaceutical composition is a liquid, for example variant CD80 polypeptides dissolved in an aqueous solution (such as physiological saline or Ringer's solution). In some embodiments, the pH of the pharmaceutical composition is between about 4.0 and about 8.5 (such as between about 4.0 and about 5.0, between about 4.5 and about 5.5, between about 5.0 and about 6.0, between about 5.5 and about 6.5, between about 6.0 and about 7.0, between about 6.5 and about 7.5, between about 7.0 and about 8.0, or between about 7.5 and about 8.5).

In some embodiments, the pharmaceutical composition comprises a pharmaceutically-acceptable excipient, for example a filler, binder, coating, preservative, lubricant, flavoring agent, sweetening agent, coloring agent, a solvent, a buffering agent, a chelating agent, or stabilizer. Examples of pharmaceutically-acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically-acceptable binders include polyvinylpyrrolidone, starch, lactose, xylitol, sorbitol, maltitol, gelatin, sucrose, polyethylene glycol, methyl cellulose, or cellulose. Examples of pharmaceutically-acceptable coatings include hydroxypropyl methylcellulose (HPMC), shellac, corn protein zein, or gelatin. Examples of pharmaceutically-acceptable disintegrants include polyvinylpyrrolidone, carboxymethyl cellulose, or sodium starch glycolate. Examples of pharmaceutically-acceptable lubricants include polyethylene glycol, magnesium stearate, or stearic acid. Examples of pharmaceutically-acceptable preservatives include methyl parabens, ethyl parabens, propyl paraben, benzoic acid, or sorbic acid. Examples of pharmaceutically-acceptable sweetening agents include sucrose, saccharine, aspartame, or sorbitol. Examples of pharmaceutically-acceptable buffering agents include carbonates, citrates, gluconates, acetates, phosphates, or tartrates.

In some embodiments, the pharmaceutical composition further comprises an agent for the controlled or sustained release of the product, such as injectable microspheres, bio-erodible particles, polymeric compounds (polylactic acid, polyglycolic acid), beads, or liposomes.

In some embodiments, the pharmaceutical composition is sterile. Sterilization may be accomplished by filtration through sterile filtration membranes or radiation. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

In some embodiments, the compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

A pharmaceutically acceptable carrier may be a pharmaceutically acceptable material, composition, or vehicle that is involved in carrying or transporting cells of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

### IV. THERAPEUTIC APPLICATIONS

Provided herein are methods for using and uses of the provided molecules containing a variant CD80 IgSF domain fusion protein described herein and pharmaceutical compositions containing the same. Such methods and uses include methods for modulating an immune response, including in connection with treating a disease or condition in a subject, such as in a human patient. Included among such molecules in the methods for using and uses herein are formats in which an extracellular domain or portion thereof of a CD80 variant polypeptide containing an affinity modified IgSF domain (e.g. IgV) is linked, directly or indirectly, to a multimerization domain, e.g. an Fc domain or region.

In particular embodiments, the full extracellular domain containing the IgV and IgC domains are linked to the multimerization domain, e.g. an Fc domain or region. In some embodiments, such a therapeutic agent is a variant CD80-Fc fusion protein, such as a variant CD80 IgV-Fv fusion protein.

In other particular embodiments as described, the Fc domain or region has effector activity. In some embodiments, such a therapeutic agent is a variant CD80-Fc fusion protein, such as a variant CD80 ECD-Fc fusion protein

In some aspects, such methods and uses include therapeutic methods and uses, for example, involving administration of the molecules or compositions containing the same, to a subject having a disease or condition in need of treatment thereof. The pharmaceutical compositions described herein (including pharmaceutical composition comprising the variant CD80 IgSF domain fusion proteins) can be used in a variety of therapeutic applications, such as for the treatment of a tumor or a cancer in a subject, viral infection or bacterial infection. In some embodiments, the disease or condition is a cancer. In some embodiments, the molecule, cell, and/or composition is administered in an effective amount to effect treatment of the disease or disorder. Uses include uses of the variant CD80 IgSF domain fusion proteins, alone or as a combination therapy as described, in such methods and treatments, and in the preparation of a medicament in order to carry out such therapeutic methods. In some embodiments, the methods are carried out by administering the variant CD80 IgSF domain fusion proteins, or compositions comprising the same, to the subject having or suspected of having the disease or condition. In some embodiments, the methods thereby treat the disease or condition or disorder in the subject.

In some aspects, the molecules or compositions pharmaceutical composition can modulate, such as increase, an immune response to treat the disease. In some embodiments, the methods carried out with a variant CD80 IgSF domain fusion protein as described increases an immune response in a subject. Among the provided methods are methods involving delivery of variant CD80 IgSF domain fusion proteins with increased affinity for CD28, which can agonize signaling of the stimulatory signal and/or increased affinity for PD-L1 and/or CTLA-4, which can antagonize signaling of an inhibitory receptor, such as block an inhibitory signal in the cell that may occur to decrease response to an activating stimulus, e.g., CD3 and/or CD28 costimulatory signal or a mitogenic signal. In some cases, the result of this can be to increase the immune response. In some embodiments, agonism of CD28, which can be dependent on or enhanced by Fc binding, may be useful to promote immunity in oncology, such as for treatment of tumors or cancers. In some embodiments, the agonism of CD28 and antagonism of PD-L1 may be useful to promote immunity in oncology, such as for treatment of tumors or cancers. In some embodiments, the agonism of CD28 and antagonism of CTLA-4 may be useful to promote immunity in oncology, such as for treatment of tumors or cancers. In some embodiments, the agonism of CD28 and antagonism of PD-L1 and CTLA-4 may be useful to promote immunity in oncology, such as for treatment of tumors or cancers.

Among the provided methods are methods involving delivery of variant CD80 IgSF domain fusion proteins which, in some embodiments, have increased affinity for CTLA-4 and/or PD-L1, which can antagonize signaling of an inhibitory receptor, such as block an inhibitory signal in the cell that may occur to decrease response to an activating stimulus, e.g., CD3 and/or CD28 costimulatory signal or a mitogenic signal. In certain cases, a variant CD80 IgSF fusion protein is capable of binding the PD-L1 on a tumor cell or APC, thereby blocking the interaction of PD-L1 and the PD-1 inhibitory receptor to prevent the negative regulatory signaling that would have otherwise resulted from the PD-L1/PD-1 interaction. In some cases, the result of this can be to increase the immune response. In other embodiments, the provided variant CD80 IgSF domain fusion proteins exhibit activity to bind CD28, in some cases with increased affinity. In some embodiments, binding to CD28 can agonize signaling of the stimulatory signal, particularly dependent on or enhanced by CD80 co-binding to PD-L1. In some embodiments, the agonism of CD28 is by PD-L1 dependent CD28 costimulation. Such PD-L1-dependent costimulation does not require an Fc with effector function and can be mediated by an Fc fusion protein containing an effector-less or inert Fc molecule. In some cases, such variant CD80 polypeptides also can facilitate promotion of an immune response in connection with the provided therapeutic methods by blocking the PD-L1/PD-1 interaction while also binding and co-stimulating a CD28 receptor on a localized T cell. In some embodiments, the agonism of CD28 and/or antagonism of CTLA-4 or PD-L1/PD-1 may be useful to promote immunity in oncology, such as for treatment of tumors or cancers.

In some embodiments, the pharmaceutical composition can be used to inhibit growth of mammalian cancer cells (such as human cancer cells). A method of treating cancer can include administering an effective amount of any of the pharmaceutical compositions described herein to a subject with cancer. The effective amount of the pharmaceutical composition can be administered to inhibit, halt, or reverse progression of cancers. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient. Thus, the present invention provides ex vivo and in vivo methods to inhibit, halt, or reverse progression of the tumor, or otherwise result in a statistically significant increase in progression-free survival (i.e., the length of time during and after treatment in which a patient is living with cancer that does not get worse), or overall survival (also called “survival rate;” i.e., the percentage of people in a study or treatment group who are alive for a certain period of time after they were diagnosed with or treated for cancer) relative to treatment with a control.

The cancers that can be treated by the pharmaceutical compositions and the treatment methods described herein include, but are not limited to, melanoma, bladder cancer, hematological malignancies (leukemia, lymphoma, myeloma), liver cancer, brain cancer, renal cancer, breast cancer, pancreatic cancer (adenocarcinoma), colorectal cancer, lung cancer (small cell lung cancer and non-small-cell lung cancer), spleen cancer, cancer of the thymus or blood cells (i.e., leukemia), prostate cancer, testicular cancer, ovarian cancer, uterine cancer, gastric carcinoma, a musculoskeletal cancer, a head and neck cancer, a gastrointestinal cancer, a germ cell cancer, or an endocrine and neuroendocrine cancer. In some embodiments, the cancer is Ewing's sarcoma. In some embodiments, the cancer is selected from melanoma, lung cancer, bladder cancer, and a hematological malignancy. In some embodiments, the cancer is a lymphoma, lymphoid leukemia, myeloid leukemia, cervical cancer, neuroblastoma, or multiple myeloma. In some embodiments, the cancer is selected from melanoma, non-small cell lung cancer (NSCLC), renal cell carcinoma (RCC), gastric cancer, bladder cancer, diffuse large B-cell lymphoma (DLBCL), Hodgkin's lymphoma, ovarian cancer, head & neck squamous cell cancer (HNSCC), mesothelioma, and triple negative breast cancer (TNBC). In some embodiments, the cancer is selected from melanoma, gastric cancer, head & neck squamous cell cancer (HNSCC), non-small cell lung cancer (NSCLC), and triple negative breast cancer (TNBC).

In some embodiments, the pharmaceutical composition (including pharmaceutical composition comprising a variant CD80 polypeptide such as variant CD80 IgSF domain fusion proteins) is administered as a monotherapy (i.e., as a single agent) or as a combination therapy (i.e., in combination with one or more additional anticancer agents, such as a chemotherapeutic drug, a cancer vaccine, or an immune checkpoint inhibitor).

In some embodiments, the pharmaceutical composition (including pharmaceutical composition comprising a variant CD80 polypeptide such as a variant CD80 IgSF domain fusion proteins) is administered in combination with an immune checkpoint inhibitor. Immune checkpoint inhibitors can include agents that specifically bind to a checkpoint molecule other than PD-L1, such as a molecule selected from among CD25, PD-1, PD-L2, CTLA-4, LAG-3, TIM-3, 4-1BB, GITR, CD40, CD40L, OX40, OX40L, CXCR2, B7-H3, B7-H4, BTLA, HVEM, CD28 and VISTA. In some embodiments, the immune checkpoint inhibitor is and antibody or antigen-binding fragment, a small molecule or a polypeptide. In some embodiments, the pharmaceutical composition is administered in combination with a PD-1 inhibitor, such as an anti-PD-1 antibody. In some embodiments, the pharmaceutical composition is administered in combination with a CTLA-4 inhibitor, such as an anti-CTLA-4 antibody.

In some embodiments, the pharmaceutical composition (including pharmaceutical composition comprising a variant CD80 polypeptide such as a variant CD80 IgSF domain fusion proteins) is administered as a combination therapy with radiation chemotherapy.

In some embodiments, the pharmaceutical composition (including pharmaceutical composition comprising a variant CD80 polypeptide such as a variant CD80 IgSF domain fusion proteins) is administered in combination with one or more chemotherapeutic agents. Exemplary chemotherapeutic agents that may be combined with the in methods provided herein include, but are not limited to, capectiabine, cyclophosphamide, dacarbazine, temozolomide, cyclophosphamide, docetaxel, doxorubicin, daunorubicin, cisplatin, carboplatin, epirubicin, eribulin, 5-FU, gemcitabine, irinotecan, ixabepilone, methotrexate, mitoxantrone, oxaliplatin, paclitaxel, nab-paclitaxel, ABRAXANE (Registered trademark) (protein-bound paclitaxel), pemetrexed, vinorelbine, and vincristine.

In some embodiments, the provided method, including provided combination therapy methods, enhances an immune response in the subject. In some embodiments, the provided methods, including the provided combination therapy methods, results in activation of T cells in the subject. In some embodiments, the provided methods, including provided combination therapy methods, reduces tumor size in a subject with cancer. In some embodiments, the provided methods, including provided combination therapy methods, can result in or achieve a reduction in size for a tumor or an eradication of tumors. In some embodiments, the mammal is a human.

The efficacy of the provided therapeutic methods, including combination therapy, can be evaluated according to guidelines that provide an objective response criteria for evaluating anti-tumor therapeutics. Such guidelines are known to a skilled artisan. For example, published guidelines include those published by the World Health Organization (WHO) (see World Health Organization, “WHO Handbook for Reporting Results of Cancer Treatment,” (1979) WHO Offset Publication No. 48, Geneva pp. 1-45 and Miller et al., (1981) Cancer. 47:207-214), and those published as Response Evaluation Criteria in Solid Tumors (RECIST) (Eisenhauer et al, (2009) Eur J Cancer. 45(2):228-247). These guidelines are provided to define when tumors in cancer patients improve (“respond”), stay the same (“stabilize”), or worsen (“progress”) during treatments. The tumors can be measured by any reproducible method. For example, CT (computed tomography) or MRI (magnetic resonance imaging) with cuts of 10 mm or less in slice thickness, or spiral CT using a 5 mm continuous reconstruction algorithm, can be used to measure tumor size. In some examples, the tumors can be measured by chest X-ray or ultrasound. It can also be possible to measure tumors using endoscopy or laparoscopy.

A variety of means are known for determining if administration of a therapeutic composition of the invention sufficiently modulates immunological activity by inducing, generating, or turning on immune cells that mediate or are capable of mediating a protective immune response; changing the physical or functional properties of immune cells; or a combination of these effects. Examples of measurements of the modulation of immunological activity include, but are not limited to, examination of the presence or absence of immune cell populations (using flow cytometry, immunohistochemistry, histology, electron microscopy, polymerase chain reaction (PCR)); measurement of the functional capacity of immune cells including ability or resistance to proliferate or divide in response to a signal (such as using T-cell proliferation assays and pepscan analysis based on 3H-thymidine incorporation following stimulation with anti-CD3 antibody, anti-T-cell receptor antibody, anti-CD28 antibody, calcium ionophores, PMA (phorbol 12-myristate 13-acetate) antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (e.g., by flow cytometry, enzyme-linked immunosorbent assays, Western blot analysis, protein microarray analysis, immunoprecipitation analysis); measurement of biochemical markers of activation of immune cells or signaling pathways within immune cells (e.g., Western blot and immunoprecipitation analysis of tyrosine, serine or threonine phosphorylation, polypeptide cleavage, and formation or dissociation of protein complexes; protein array analysis; DNA transcriptional, profiling using DNA arrays or subtractive hybridization); measurements of cell death by apoptosis, necrosis, or other mechanisms (e.g., annexin V staining, TUNEL assays, gel electrophoresis to measure DNA laddering, histology; fluorogenic caspase assays, Western blot analysis of caspase substrates); measurement of the genes, proteins, and other molecules produced by immune cells (e.g., Northern blot analysis, polymerase chain reaction, DNA microarrays, protein microarrays, 2-dimensional gel electrophoresis, Western blot analysis, enzyme linked immunosorbent assays, flow cytometry); and measurement of clinical symptoms or outcomes for example, by measuring relapse rate or disease severity (using clinical scores known to the ordinarily skilled artisan).

A. Dosing and Administration

In some embodiments, a pharmaceutical composition described herein (including pharmaceutical composition comprising the variant CD80 IgSF domain fusion proteins) is administered to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage can be determined in light of factors related to the subject requiring treatment. Dosage and administration can be adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

In some embodiments, modeling and simulation of pharmacokinetic (PK) and pharmacodynamic (PD) profiles observed in control animals and animal models of disease (e.g., cancer models) can be used to predict or determine patient dosing. For example, PK data from non-human primates (e.g., cynomolgus monkeys) can be used to estimate human PK. Similarly, mouse PK and PD data can be used to predict human dosing. The observed animal data can be used to inform computational models which can be used to simulate human dose response. In some embodiments, transduction models, such as signal distribution models (SDM; Lobo E D et al., AAPS PharmSci. 2002; 4(4): E42) or cell distribution models (CDM; Yang J et al., AAPS J. 2010; 12(1):1-10) can be informed by such PK and PD animal data (see, e.g., Example 26) and used to predict human dosing and response. In some embodiments, transduction models, such as SDM, can be used to predict human dosing and administration. In some embodiments, transduction models, such as SDM, can be used to develop immuno-oncology therapies, such as therapies including treatment with variant CD80 fusion proteins described herein. In some embodiments, the model is an SDM. In some embodiments, the model is a CDM. In some embodiments, transduction models, such as SDM, can be used to determine tumor static concentration (TSC), which refers to the minimum drug concentration where the tumor is neither growing nor regressing. In some embodiments, TSC can be used, for example alone or in combination with PK data, to determine (e.g., predict) human dosing. For example, to induce tumor growth inhibition, human dosing may be higher or delivered in a regimen that results in the drug concentration exceeding the predicted TSC.

Long-acting pharmaceutical compositions may be administered every 3 to 4 days, every week, biweekly, every three weeks, once a month, etc. depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data. A number of biomarkers or physiological markers for therapeutic effect can be monitored including T cell activation or proliferation, cytokine synthesis or production (e.g., production of TNF-α, IFN-γ, IL-2), induction of various activation markers (e.g., CD25, IL-2 receptor), inflammation, joint swelling or tenderness, serum level of C-reactive protein, anti-collagen antibody production, and/or T cell-dependent antibody response(s).

Typically, precise amount of the compositions of the present invention to be administered can be determined by a physician with consideration of individual differences in age, weight, tumor size, extent of infection or metastasis, and condition of the patient (subject). In some embodiments, when referencing dosage based on mg/kg of the subject, an average human subject is considered to have a mass of about 70 kg-75 kg, such as 70 kg and a body surface area (BSA) of 1.73 m².

In some embodiments, the dosage, such as to achieve a therapeutically effective amount, of the pharmaceutical composition (including pharmaceutical composition comprising the variant CD80 IgSF domain fusion proteins) is a single dose or a repeated dose, such as via administration of multiple doses. In some embodiments, the doses are given to a subject once per day, twice per day, three times per day, or four or more times per day. In some embodiments, about 1 or more (such as about 2 or more, about 3 or more, about 4 or more, about 5 or more, about 6 or more, or about 7 or more) doses are given in a week. In some embodiments, multiple doses are given over the course of days, weeks, months, or years. In some embodiments, a course of treatment is about 1 or more doses (such as about 2 or more doses, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In some embodiments, an administered dose of the pharmaceutical composition (including pharmaceutical composition comprising the variant CD80 IgSF domain fusion proteins) is about 1 μg of protein per kg subject body mass or more (such as about 2 μg of protein per kg subject body mass or more, about 5 μg of protein per kg subject body mass or more, about 10 μg of protein per kg subject body mass or more, about 25 μg of protein per kg subject body mass or more, about 50 μg of protein per kg subject body mass or more, about 100 μg of protein per kg subject body mass or more, about 250 μg of protein per kg subject body mass or more, about 500 μg of protein per kg subject body mass or more, about 1 mg of protein per kg subject body mass or more, about 2 mg of protein per kg subject body mass or more, or about 5 mg of protein per kg subject body mass or more).

In some embodiments, the pharmaceutical composition (including pharmaceutical composition comprising the variant CD80 IgSF domain fusion proteins) is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, intraspinally, intratumorally or systemically.

In some embodiments, the pharmaceutical composition (including pharmaceutical composition comprising the variant CD80 IgSF domain fusion proteins) is administered parenterally. Examples provided herein demonstrate that particularly suitable routes of administration include intravenous, subcutaneous or intratumoral administration. In some embodiments, the pharmaceutical composition is in a form suitable for administration by injection, such as by bolus injection. In some embodiments, the pharmaceutical composition is in a form suitable for infusion injection, for example by intravenous injection. In some embodiments, the infusion duration is, is at least, or is about 30 minutes, 40 minutes, 50 minutes, 1 hour, 1.5 hours, 2 hours, 3 hours, 4 hours, 5 hours or 6 hours. In some embodiments the infusion duration is between about 30 minutes and 6 hours. In some embodiments, the infusion duration is between about 30 minutes and 5 hours. In some embodiments, the infusion duration is between about 30 minutes and 4 hours. In some embodiments, the infusion duration is between about 30 minutes and 3 hours. In some embodiments, the infusion duration is between about 30 minutes and 2 hours. In some embodiments, the infusion duration is between about 30 minutes and 1 hour. In some embodiments, the infusion duration is or is about 30 minutes.

In some embodiments, a pharmaceutical composition (including a pharmaceutical composition comprising the variant CD80 IgSF domain fusion proteins) is administered in a therapeutically effective amount to treat a cancer in a subject that is known or suspected of having a cancer. In some embodiments, the therapeutically effective amount is between about 0.001 mg/kg and about 100 mg/kg, inclusive. In some embodiments, the therapeutically effective amount is between about 0.003 mg/kg and about 80 mg/kg, inclusive. In some embodiments, the therapeutically effective amount is between about 0.5 mg/kg and about 60 mg/kg, inclusive. In some embodiments, the therapeutically effective amount is between about 1 mg/kg and about 60 mg/kg, inclusive. In some embodiments, the therapeutically effective amount is between about 1 mg/kg and about 40 mg/kg, inclusive. In some embodiments, the therapeutically effective amount is between about 1 mg/kg and about 20 mg/kg, inclusive.

In some embodiments, a pharmaceutical composition (including pharmaceutical composition comprising the variant CD80 IgSF domain fusion proteins) is administered in a therapeutically effective amount to treat a cancer in a subject that is known or suspected of having a cancer. In some embodiments, the therapeutically effective amount is an amount between or between about 1 mg/kg and 10 mg/kg, inclusive, such as between or between about 1 mg/kg and 8 mg/kg, between or between about 1 mg/kg and 6 mg/kg, between or between about 1 mg/kg and 4 mg/kg, between or between about 1 mg/kg and 2 mg/kg, between or between about 2 mg/kg an 10 mg/kg, between or between about 2 mg/kg and 8 mg/kg, between or between about 2 mg/kg and 6 mg/kg, between or between about 2 mg/kg and 4 mg/kg, between or between about 4 mg/kg and 10 mg/kg, between or between about 4 mg/kg and 8 mg/kg, between or between about 4 mg/kg and 6 mg/kg, between or between about 6 mg/kg and 10 mg/kg, between or between about 6 mg/kg and 8 mg/kg or between or between about 8 mg/kg and 10 mg/kg, each inclusive.

In some embodiments, the therapeutically effective amount is the amount, e.g., amount of variant CD80 fusion protein as described herein, needed to saturate at least 16% of CD28 receptors. In some embodiments, the therapeutically effective amount is the amount, e.g., amount of variant CD80 fusion protein as described herein, needed to saturate at least 20% of CD28 receptors. In some embodiments, the therapeutically effective amount is the amount, e.g., amount of variant CD80 fusion protein as described herein, needed to saturate at least 30% of CD28 receptors. In some embodiments, the therapeutically effective amount is the amount, e.g., amount of variant CD80 fusion protein as described herein, needed to saturate at least 40% of CD28 receptors. In some embodiments, the therapeutically effective amount is the amount, e.g., amount of variant CD80 fusion protein as described herein, needed to saturate at least 50% of CD28 receptors. In some embodiments, the therapeutically effective amount is the amount, e.g., amount of variant CD80 fusion protein as described herein, needed to saturate at least 60% of CD28 receptors. In some embodiments, the therapeutically effective amount is the amount, e.g., amount of variant CD80 fusion protein as described herein, needed to saturate at least 70% of CD28 receptors. In some embodiments, the therapeutically effective amount is the amount, e.g., amount of variant CD80 fusion protein as described herein, needed to saturate at least 80% of CD28 receptors. In some embodiments, the therapeutically effective amount is the amount, e.g., amount of variant CD80 fusion protein as described herein, needed to saturate at least 90% of CD28 receptors. In some embodiments, the therapeutically effective amount is the amount, e.g., amount of variant CD80 fusion protein as described herein, needed to saturate at least 95% of CD28 receptors. In some embodiments, the therapeutically effective amount is the amount, e.g., amount of variant CD80 fusion protein as described herein, needed to saturate at least 99% of CD28 receptors.

In some embodiments, the pharmaceutical composition (including pharmaceutical composition comprising the variant CD80 IgSF domain fusion proteins) is in a form suitable for administration by intratumoral delivery. In some aspects, a dosage amount for intratumoral delivery is less than the amount administered by injection or other parenteral routes.

In some embodiments the therapeutically effective amount of a pharmaceutical composition (including pharmaceutical composition comprising the variant CD80 IgSF domain fusion proteins) for intratumoral administration is an amount between or between about 0.1 mg/kg and 1 mg/kg, inclusive, such as between or between about 0.1 mg/kg and 0.8 mg/kg, between or between about 0.1 mg/kg and 0.6 mg/kg, between or between about 0.1 mg/kg and 0.4 mg/kg, between or between about 0.1 mg/kg and 0.2 mg/kg, between or between about 0.2 mg/kg an 1 mg/kg, between or between about 0.2 mg/kg and 0.8 mg/kg, between or between about 0.2 mg/kg and 0.6 mg/kg, between or between about 0.2 mg/kg and 0.4 mg/kg, between or between about 0.4 mg/kg and 1 mg/kg, between or between about 0.4 mg/kg and 0.8 mg/kg, between or between about 0.4 mg/kg and 0.6 mg/kg, between or between about 0.6 mg/kg and 1 mg/kg, between or between about 0.6 mg/kg and 0.8 mg/kg or between or between about 0.8 mg/kg and 1 mg/kg, each inclusive.

In some embodiments, the therapeutically effective amount of a pharmaceutical composition (including pharmaceutical composition comprising the variant CD80 IgSF domain fusion proteins) is administered as a single dose.

In some embodiments, the therapeutically effective amount of a pharmaceutical composition (including pharmaceutical composition comprising the variant CD80 IgSF domain fusion proteins) is administered as multiple doses, such as two or more doses, for example, 2, 3, 4, 5 or 6 doses. In some embodiments, the therapeutically effective amount of a pharmaceutical composition (including pharmaceutical composition comprising the variant CD80 IgSF domain fusion proteins) is administered in six or fewer multiple doses. In some embodiments, the therapeutically effective amount of a pharmaceutical composition is administered as two doses. In some embodiments, the therapeutically effective amount of a pharmaceutical composition is administered as three doses. In some embodiments, the therapeutically effective amount of a pharmaceutical composition is administered as four doses. In some embodiments, the therapeutically effective amount of a pharmaceutical composition is administered as five doses. In some embodiments, the therapeutically effective amount of a pharmaceutical composition is administered as six doses. In some embodiments, the multiple doses are administered at least or about at least one week apart. In some embodiments, the doses are administered once weekly (QW or Q1W), once every 2 weeks (Q2W), once every 3 weeks (Q3W) or once every 4 weeks (Q4W). In some embodiments, the interval between each administered dose is or is about one week. In some embodiments, the interval between each administered dose or is is about 2 weeks. In some embodiments, the interval between each administered dose is or is about 3 weeks. In some embodiments, the interval between each administered dose is or is about 4 weeks.

In some embodiments, the dose, e.g., single dose or each individual dose of multiple doses (e.g., six or fewer multiple doses), is an amount between about 0.001 mg/kg and about 100 mg/kg, inclusive. In some embodiments, the dose, e.g., single dose or each individual dose of multiple doses (e.g., six or fewer multiple doses), is an amount between about 0.003 mg/kg and about 80 mg/kg, inclusive. In some embodiments, the dose, e.g., single dose or each individual dose of multiple doses (e.g., six or fewer multiple doses), is an amount between about 0.5 mg/kg and about 60 mg/kg, inclusive. In some embodiments, the dose, e.g., single dose or each individual dose of multiple doses (e.g., six or fewer multiple doses), is an amount between about 1 mg/kg and about 60 mg/kg, inclusive. In some embodiments, the dose, e.g., single dose or each individual dose of multiple doses (e.g., six or fewer multiple doses), is an amount between about 1 mg/kg and about 40 mg/kg, inclusive. In some embodiments, the dose, e.g., single dose or each individual dose of multiple doses (e.g., six or fewer multiple doses), is an amount between about 1 mg/kg and about 20 mg/kg, inclusive. In some embodiments, the dose, e.g., single dose or each individual dose of multiple doses (e.g., six or fewer multiple doses), is an amount between about 1 mg/kg and about 10 mg/kg, inclusive. In some embodiments, the dose, e.g., single dose or each individual dose of multiple doses (e.g., six or fewer multiple doses), is an amount between about 1 mg/kg and about 8 mg/kg, inclusive. In some embodiments, the dose, e.g., single dose or each individual dose of multiple doses (e.g., six or fewer multiple doses), is an amount between about 1 mg/kg and about 6 mg/kg, inclusive. In some embodiments, the dose, e.g., single dose or each individual dose of multiple doses (e.g., six or fewer multiple doses), is an amount between about 1 mg/kg and about 3 mg/kg, inclusive. In some embodiments, the dose, e.g., single dose or each individual dose of multiple doses (e.g., six or fewer multiple doses), is an amount of about 1 mg/kg, 3 mg/kg, or 10 mg/kg.

In some embodiments, when the dose is administered once weekly, such as QIW, the amount administered per dose is between about 1 mg/kg and about 3 mg/kg. In some embodiments, when the dose is administered once weekly, such as QIW, the amount administered per dose is or is about 1 mg/kg, 1.5 mg/kg, 2 mg/kg, 2.5 mg/kg, or 3 mg/kg, or any value in between any of the foregoing.

In some embodiments, when the dose is administered once every 3 weeks, such as Q3W, the amount administered per dose is between about 3 mg/kg and about 10 mg/kg. In some embodiments, when the dose is administered once every 3 weeks, such as Q3W, the amount administered per dose is or is about 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, or 10 mg/kg, or any value between.

In some embodiments, a dose regimen as described herein is administered to achieve a therapeutically effective amount.

In some embodiments, the duration of administration, such as for administration of the multiple doses (e.g., six or fewer single doses), is for one week, two weeks, three weeks, one month, two months, three months, four months, five months, or six months. In some embodiments, the duration of administration, such as for administration of the multiple doses (e.g., six or fewer single doses), is for no more than two months, such as no more than six weeks.

In some embodiments, the therapeutically effective amount, such as administered as 2, 3, 4, 5 or 6 doses, is administered within a period of no more than 6 weeks, such as within a period of 1 week to 6 weeks. In some embodiments, the therapeutically effective amount is administered within a period of six weeks. In some embodiments, the therapeutically effective amount is administered within a period of five weeks. In some embodiments, the therapeutically effective amount is administered within a period of four weeks. In some embodiments, the therapeutically effective amount is administered within a period of three weeks. In some embodiments, the therapeutically effective amount is administered within a period of two weeks. In some embodiments, the therapeutically effective amount is administered within a period of one week.

It is contemplated that dosing (e.g., multiple doses), can continue until any time as desired by a skilled practitioner. For example, dosing may continue until a desirable disease response is achieved, such as a reduction in tumor size, a reduction or amelioration in signs and/or symptoms of a disease.

B. Combination Therapy

In some embodiments, the fusion proteins containing variant CD80 polypeptides or pharmaceutical compositions thereof can also be administered with one or more additional agents. In particular embodiments, the one or more additional agent is an agent that does not compete with or block the binding of the variant CD80 polypeptide to its cognate binding partner, such as to one or more of CD28, CTLA-4 and PD-L1. For example, in particular embodiments, the variant CD80 polypeptide of the fusion protein for use in methods provided herein binds to PD-L1, such as with increased affinity compared to the wild-type or unmodified CD80 polypeptide, and the additional agent does not bind to PD-L1 and/or does not compete for binding to PD-L1 or does not share the same or overlapping epitope of PD-L1 as the variant CD80 polypeptide.

In some embodiments, the combination therapy includes administering to a subject a therapeutically effective amount of the anti-cancer agent, such as any described herein. In some embodiments, a therapeutically effective dose can be from or from about 0.01 mg to 1000 mg, such as a dose of at least 0.01 mg, 0.1 mg, 1 mg, 10 mg, 1000 mg, 2000 mg, 3000 mg or more. In some embodiments, a therapeutically effective dose of the anti-cancer agent is from or from about 0.01 mg/kg to about 50 mg/kg, such as about 0.1 mg/kg to about 20 mg/kg, about 0.1 to about 10 mg/kg, about 0.3 to about 10 mg/kg, about 0.5 mg/kg to about 5 mg/kg or about 0.5 mg/kg to about 1 mg/kg.

In some embodiments, the dose of the anti-cancer agent (e.g. immune checkpoint inhibitor or chemotherapeutic agent) is continued or repeated in accord with its clinically dosing schedule. Thus, in some embodiments, in a dose schedule or cycle of administration in accord with the provided methods, the variant CD80 polypeptide (e.g. variant CD80-Fc fusion protein) can be administered only one time, such as in a single dose or infusion or in several doses as described, whereas the administration of the anticancer agent is continued or repeated more than one time, such as three times a week, two times a week, once a week, once every two weeks, once every three weeks or once a month during a dosing schedule or cycle of administration. In some embodiments, the dosing schedule or cycle of administration is or is about 28 days or 4 weeks.

In some embodiments, the anti-cancer agent is an immune checkpoint inhibitor. The immune checkpoint inhibitor can be administered in an amount that is from or from about 0.01 mg to 1000 mg, such as at a dose of at least 0.01 mg, 0.1 mg, 1 mg, 10 mg, 1000 mg, 2000 mg, 3000 mg or more. In an exemplary embodiment, an immune checkpoint inhibitor may be administered at about 0.3 mg/kg to 10 mg/kg, or the maximum tolerated dose, such as at least 0.5 mg/kg, or at least 1 mg/kg, or at least 2 mg/kg, or at least 3 mg/kg, or at least 5 mg/kg, or at least 8 mg/kg. In some cases, the dose can be administered as a single dose or in a plurality of doses. Alternatively, the immune checkpoint inhibitor may be administered by an escalating dosage regimen including administering a first dosage at about 3 mg/kg, a second dosage at about 5 mg/kg, and a third dosage at about 9 mg/kg. Alternatively, the escalating dosage regimen includes administering a first dosage of the immune checkpoint inhibitor at about 5 mg/kg and a second dosage at about 9 mg/kg. Another stepwise escalating dosage regimen may include administering a first dosage of an immune checkpoint inhibitor at about 3 mg/kg, a second dosage of about 3 mg/kg, a third dosage of about 5 mg/kg, a fourth dosage of about 5 mg/kg, and a fifth dosage of about 9 mg/kg. In another aspect, a stepwise escalating dosage regimen may include administering a first dosage of 5 mg/kg, a second dosage of 5 mg/kg, and a third dosage of 9 mg/kg. In some embodiments, particular dosages can be administered twice weekly, once weekly, once every two weeks, once every three weeks or once a month or more. In some cases, the dosages can be administered over a course of a cycle that can be repeated, such as repeated for one month, two months, three months, six months, 1 year or more.

In some embodiments, the additional agent is a checkpoint inhibitor that is able to block the interaction between PD-L1 and its receptor PD-1, thereby providing an alternative or approach for blocking or preventing the negative regulatory signaling that would have otherwise resulted from the PD-L1/PD-1 interaction.

In some embodiments, targeting blockade of such receptor/ligand interactions achieved by the provided combination therapy methods can produce additive or synergistic antitumor activities. Hence, in some aspects, the provided combination therapy improves the treatment outcome or response compared to treatment of the subject, or a group of similarly situated subjects, with either molecule alone as a monotherapy. In some aspects, the provided combination therapy achieves similar or greater anti-tumor efficacy at lower dosages of one or other molecules compared to treatment of the subject, or a group of similarly situated subjects, with either molecule alone as a monotherapy.

In some embodiments, the additional agent is a PD-1 inhibitor. PD-1 is an inhibitory receptor that is a type 1 membrane protein and is able to be bound by ligands such as PD-L1 and PD-L2, which are members of the B7 family. PD-1 includes human and non-human proteins. In particular, PD-1 antigen includes human PD-1 (see e.g., UniProt Accession No. Q15116.3). In some embodiments, a PD-1 inhibitor useful in the provided combinations described herein include any molecule capable of inhibiting, blocking, abrogating or interfering with the activity or expression of PD-1 In some aspects, a PD-1 inhibitor disrupts the interaction between PD-1 and one or both of its ligands PD-L1 and PD-L2.

In some embodiments, the PD-1 inhibitor is a small molecule, a nucleic acid, a protein or polypeptide, an antibody or antigen-binding fragment thereof, a peptibody, a diabody, or a minibody. In one instance the PD-1 inhibitor is a small molecule compound (e.g., a compound having a molecule weight of less than about 1000 Da.). Examples of small molecule inhibitor sof PD-1 (e.g. Sasikumar et al., Biodrugs (2018) 10.1007/s40259-018-0303-4). In other instances, useful PD-1 inhibitors in the combinations described herein include nucleic acids and polypeptides. A nonlimiting exemplary peptide that is a PD-1 inhibitor is AUR-012. A PD-1 inhibitor can be a polypeptide (e.g., macrocyclic polypeptide), such as those exemplified in U.S. Patent Application Publication No.: 2014/0294898, In other examples, a PD-1 inhibitor can include a recombinant fusion protein of an extracellular domain of a PD-1 ligand, such as an extracellular domain of PD-L1 or PD-L2. For example, AMP-224 (Amplimmune/GlaxoSmithKline) contains the extracellular domain of PD-L2 and an Fc region of human IgG, which can bind to PD-1 and block interactions with its ligands, se e.g, international patent application publication Nos. WO2010/027827 and WO2011/066342.

Exemplary inhibitors of PD-1 include, but are not limited to CS1003 (Cstone Pharmaceuticals), AK103 or AK105 (Akesio Biopharma, Hangzhou Hansi Biologics, Hanzhong Biologics), HLX-10 (Henlius Biotech). LZM009 (Livzon), JTX-4014.

In some embodiments, the PD-1 inhibitor is an anti-PD-1 antibody or antigen binding fragments thereof. In some aspects, anti-PD-1 antibody or antigen-binding fragments can exhibit one or more of the following characteristics: (a) binds to human PD-1 with a KD of 1×10⁻⁷ M or less, such as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-gamma production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and/or (j) inhibits tumor cell growth in vivo.

In some cases, the anti-PD-1 antibody is a chimeric antibody. In other cases, the anti-PD-1 antibody is a humanized antibody. In further cases, the anti-PD-1 antibody is a chimeric humanized antibody. The anti-PD-1 antibody can be a human antibody or humanized antibody. Examples of anti-PD-1 antibodies or antigen-binding fragments are known, see e.g. U.S. Pat. Nos. U.S. Pat. Nos. 6,808,710, 7,488,802, 7,943,743, 8,008,449, 8,168,757 and 8,354,509, 8,779, 105, 8,735, 553; U.S. Patent Application Publication US20050180969, US20070166281, US20170290808, international patent application publication Nos. WO2008156712 WO2012145493, WO2018156494, WO201891661, WO2014206107; Clinical Trial Study Record Nos.: NCT03474640; NCT03473743; NCT03311412; NCT02383212. In some embodiments, two or more PD-1 antibodies are administered in combination with a variant CD80 fusion protein as described herein.

Exemplary anti-PD-1 antibodies include, but are not limited to, AGEN-2034 (Agenus), AM-0001, AK 103 (Akeso Biopharma), BAT-I306 (Bio-Thera Solutions), BGB-A317 (Beigene), BI-754091, cemiplimab (REGN2810 or SAR439684) (Sanofi/Regeneron), CBT-501, ENUM-244C8, GB-226, GLS-010 (Gloria Pharmaceuticals; WuXi Biologics), GX-D1, IBI308 (Innovent Biologics), JS001 (Junshi Biosciences), JNJ-63723283, MGA012 (Macrogenics), MEDI0680 or AMP514 (AstraZeneca/MedImmune), nivolumab, pembrolizumab, pidilizumab (Pfizer), CT011 or MDV9300, PDR001 (Pfizer), recombinant humanized anti-PD-1 mAb (Bio-Thera Solutions), PD-1 based bispecific antibody (Beijing Hanmi Pharmaceutical), PD-1 monoclonal antibody (Genor Biopharma), REGN-2810, SHR-1210 (Hengrui Medicine), Sym021, SSI-361, TAB001, TSR-042 or an antigen binding fragment thereof.

In one embodiment, the anti-PD-1 Ab is nivolumab or a derivative thereof, such as variants or antigen-binding fragments of nivolumab. Nivolumab (also known as Opdivorm; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (see e,g, U.S. Pat. No. 8,008,449; Wang et al., 2014 Cancer Immunol Res. 2(9):846-56).

In another embodiment, the anti-PD-1 antibody is pembrolizumab or a derivative thereof, such as variants or antigen-binding fragments of pembrolizumab. Pembrolizumab (also known as Keytruda™, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. No. 8,900,587 and as antibody designated h409AII in International patent publication No. WO2008156712.

In a further embodiment, the anti-PD-1 antibody is pidilizumab (also called hBAT-1 or CT-011) or derivatives thereof, such as variants or antigen-binding fragments of pidilizumab. Pidilizumab is a humanized IgG1K monoclonal antibody that was generated from a murine antibody (BAT), which was raised against B lymphoid cell membranes, and has been shown to elicit T-celland NK-cell-based activities. Pidilizumab binds human PD-1 (see, e.g., antibody designated BAT-RK_{D}/RHC in US 2005/0180969).

In other embodiments, the anti-PD-1 Ab is MEDI0608 (formerly AMP-514), or is a derivative thereof, such as variants or antigen-binding fragment of MEDI1068. MEDI0608 is a monoclonal antibody against the PD-1 receptor. MEDI0608 is described, for example, in U.S. Pat. No. 8,609,089B2.

In some embodiments, the additional agent is a checkpoint inhibitor that is able to block the interaction between CTLA-4 and its cognate binding partners CD80 or CD86. Exemplary anti-CTLA-4 antibodies include ipilimumab (Bristol-Myers Squibb) and tremelimumab (Pfizer).

In some embodiments, the anti-CTLA-4 Ab is ipilimumab (also called MDX-010, MDX-101, MDX-CTLA-4, 10D1 or Yervoy®), or is a derivative thereof, such as variants or antigen-binding fragments of ipilimumab. Ipilimumab is a fully humanized IgG1 monoclonal antibody against CTLA-4. Ipilimumab is described, for example, in International published PCT Appl. No. WO2001014424 or EP patent EP1503794, U.S. published patent appl. Nos. U.S. Pat. App. Pub. No. US20020086014, US20150283234.

In some embodiments, the anti-CTLA-4 Ab is tremelimumab (also called CP-675, CP-675206, ticilimumab, antibody clone 11.2.1), or is a derivative thereof, such as a variant or antigen-binding fragment of tremelimumab. Tremelimumab is a monoclonal antibody against CTLA-4. Tremelimumab is described, for example, in U.S. Pat. Nos. 6,682,736, 7,109,003; 7,123,281; 7,411,057; 7,824,679; 8,143,379; 7,807,797; and 8,491,895.

Checkpoint inhibitors, such as anti-PD-1 antibodies, for use in the combination therapy described herein include antigen-binding fragment of an antibody, e.g. anti-PD-1 antibody, such as any of the above antibodies. Examples of antigen-binding fragments include, for example, a Fab fragment, which is a monovalent fragment containing the VL, VH, CL and CHI domains; (ii) a F(ab′)2 fragment, which is a bivalent fragment comprising two Fab fragments linked by a disulfide bridge at the hinge region; (iii) a Fd fragment containing the VH and CHI domains; and (iv) a Fv fragment containing the VL and VH domains of a single arm of an antibody.

In some embodiments, the anti-cancer agent is a chemotherapeutic agent. In some embodiments, the anti-cancer agent is an alkylating agent. Alkylating agents are compounds that directly damage DNA by forming covalent bonds with nucleic acids and inhibiting DNA synthesis. Exemplary alkylating agents include, but are not limited to, mechlorethamine, cyclophosphamide, ifosamide, melphalan, chlorambucil, busulfan, and thiotepa as well as nitrosurea alkylating agents such as carmustine and lomustine. In some embodiments, the anti-cancer agent is a platinum drug. Platinum drugs bind to and cause crosslinking of DNA, which ultimately triggers apoptosis. Exemplary platinum drugs include, but are not limited to, cisplatin, carboplatin, oxaliplatin, satraplatin, picoplatin, nedaplatin, triplatin, and lipoplatin. In some embodiments, the anti-cancer agent is an antimetabolite. Antimetabolites interfere with DNA and RNA growth by substituting for the normal building blocks of RNA and DNA. These agents damage cells during the S phase, when the cell's chromosomes are being copied. In some cases, antimetabolites can be used to treat leukemias, cancers of the breast, ovary, and the intestinal tract, as well as other types of cancer. Exemplary antimetabolites include, but are not limited to, 5-fluorouracil (5-FU), 6-mercaptopurine (6-MP), capecitabine (Xeloda®), cytarabine (Ara-C®), floxuridine, fludarabine, gemcitabine (Gemzar®), hydroxyurea, methotrexate, and pemetrexed (Alimta®). In some embodiments, the anti-cancer agent is an anti-tumor antibiotic. Anti-tumor antibiotics work by altering the DNA inside cancer cells to keep them from growing and multiplying. Anthracyclines are anti-tumor antibiotics that interfere with enzymes involved in DNA replication. These drugs generally work in all phases of the cell cycle. They can be widely used for a variety of cancers. Exemplary anthracyclines include, but are not limited to, daunorubicin, doxorubicin, epirubicin, and idarubicin. Other anti-tumor antibiotics include actinomycin-D, bleomycin, mitomycin-C, and mitoxantrone. In some embodiments, the anti-cancer agent is a topoisomerase inhibitor. These drugs interfere with enzymes called topoisomerases, which help separate the strands of DNA so they can be copied during the S phase. Topoisomerase inhibitors can be used to treat certain leukemias, as well as lung, ovarian, gastrointestinal, and other cancers. Exemplary toposiomerase inhibitors include, but are not limited to, doxorubicin, topotecan, irinotecan (CPT-11), etoposide (VP-16), teniposide, and mitoxantrone. In some embodiments, the anti-cancer agent is a mitotic inhibitor. Mitotic inhibitors are often plant alkaloids and other compounds derived from natural plant products. They work by stopping mitosis in the M phase of the cell cycle but, in some cases, can damage cells in all phases by keeping enzymes from making proteins needed for cell reproduction. Exemplary mitotic inhibitors include, but are not limited to, paclitaxel (Taxol®), docetaxel (Taxotere®), ixabepilone (Ixempra®), vinblastine (Velban®), vincristine (Oncovin®), vinorelbine (Navelbine®), and estramustine (Emcyt®). In some embodiments, the anti-cancer agent is a platinum-based chemotherapeutic agent, such as oxaliplatin. Oxaliplatin is a platinum-based drug that acts as a DNA cross-linking agent to effectively inhibit DNA replication and transcription, resulting in cytotoxicity which is cell cycle non-specific.

In some embodiments, a chemotherapeutic agent, such as a platinum-based agent, e.g. oxaliplatin, is administered to a human patient in an amount that can range from about 20 mg/m² to about 150 mg/m2, for example, from about 40 mg/m² to about 100 mg/m², or an amount of at or about 50 mg/m², at or about 55 mg/m2, at or about 60 mg/m², at or about 65 mg/m², at or about 70 mg/m², at or about 75 mg/m², at or about 80 mg/m², at or about 85 mg/m², at or about 90 mg/m², or at or about 95 mg/m², or any value between any of the foregoing. In some embodiments, particular dosages can be administered twice weekly, once weekly, once every two weeks, once every three weeks or once a month or more. In some cases, the dosages can be administered over a course of a cycle that can be repeated, such as repeated for one month, two months, three months, six months, 1 year or more.

The anticancer agent, such as a checkpoint inhibitor (e.g. PD-1 inhibitor, such as an anti-PD-1 antibody or antigen-binding fragment thereof) can be administered prior to, simultaneously with or near simultaneously with, sequentially with or intermittently with the fusion proteins containing variant CD80 polypeptides or pharmaceutical compositions thereof. For example, the anticancer agent, such as a checkpoint inhibitor (e.g. PD-1 inhibitor, e.g. anti-PD-1 antibody), and the fusion protein containing variant CD80 polypeptide (e.g., variant CD80-Fc, such as variant CD80 IgV-Fc) can be co-administered together or separately. In some aspects, the fusion protein containing the variant CD80 polypeptide is administered prior to the anticancer agent, such as checkpoint inhibitor (e.g. PD-1 inhibitor). In some embodiments, the anticancer agent, such as checkpoint inhibitor (e.g. PD-1 inhibitor) is administered within 2 hours to one week after the initiation of administration of the variant CD80 fusion protein or after the administration of the last dose of a therapeutically effective amount of the variant CD80 fusion protein. In some aspects, the anticancer agent, such as checkpoint inhibitor (e.g. PD-1 inhibitor) is administered between or between about 2 hours and 144 hours after the initiation of administration of the variant CD80 fusion protein or after administration of the last dose of a therapeutically effective amount of the variant CD80 fusion protein, such as between or between about 2 hours and 120 hours, between or between about 2 hours and 96 hours, between or between about 2 hours and 72 hours, between or between about 2 hours and 48 hours, between or between about 2 hours and 24 hours, between or between about 2 hours and 12 hours, between or between about 12 hours and 120 hours, between or between about 12 hours and 96 hours, between or between about 12 hours and 72 hours, between or between about 12 hours and 48 hours, between or between about 12 hours and 24 hours, between or between about 24 hours and 120 hours, between or between about 24 hours and 96 hours, between or between about 24 hours and 72 hours, between or between about 24 hours and 48 hours, between or between about 48 hours and 120 hours, between or between about 48 hours and 96 hours, between or between about 48 hours and 72 hours, between or between about 72 hours and 120 hours, between or between about 72 hours and 96 hours or between or between about 96 hours and 120 hours.

The anticancer agent, such as checkpoint inhibitor (e.g. PD-1 inhibitor, such as anti-PD-1 antibody), can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a anticancer agent, such as checkpoint inhibitor (e.g. PD-1 inhibitor, e.g. anti-PD-1 antibody), is administered to a subject one or more times. In some embodiments, an effective dose of a anticancer agent, such as checkpoint inhibitor (e.g. PD-1 inhibitor, such as an anti-PD-1 antibody), is administered to the subject once a month, less than once a month, such as, for example, every two months or every three months. In some embodiments, an effective dose of a anticancer agent, such as checkpoint inhibitor (e.g. PD-1 inhibitor, such as an anti-PD-1 antibody), is administered less than once a month, such as, for example, once every three weeks, once every two weeks, or once every week. In some cases, an effective dose of a anticancer agent, such as checkpoint inhibitor (e.g. PD-1 inhibitor, such as an anti-PD-1 antibody), is administered to the subject at least once. In some embodiments, the effective dose of a anticancer agent, such as checkpoint inhibitor (e.g. PD-1 inhibitor, e.g. an anti-PD-1 antibody), may be administered multiple times, including for periods of at least a month, at least six months, or at least a year.

In some embodiments, pharmaceutical compositions of a anticancer agent, such as checkpoint inhibitor (e.g. PD-1 inhibitor, such as an anti-PD-1 antibody), are administered in the provided combination therapy in an amount effective for treatment of (including prophylaxis of) cancer. The therapeutically effective amount is typically dependent on the weight of the subject being treated, his or her physical or health condition, the extensiveness of the condition to be treated, or the age of the subject being treated. In general, a anticancer agent, such as checkpoint inhibitor (e.g. PD-1 inhibitor, such as an anti-PD-1 antibody), may be administered in an amount in the range of about 10 μg/kg body weight to about 100 mg/kg body weight per dose. In some embodiments, the anticancer agent, such as checkpoint inhibitor (e.g. PD-1 inhibitor, such as an anti-PD-1 antibody), may be administered in an amount in the range of about 50 μg/kg body weight to about 5 mg/kg body weight per dose. In some embodiments, a anticancer agent, such as checkpoint inhibitor (e.g. PD-1 inhibitor, such as an anti-PD-1 antibody), may be administered in an amount in the range of about 100 μg/kg body weight to about 10 mg/kg body weight per dose. In some embodiments, a anticancer agent, such as checkpoint inhibitor (e.g. PD-1 inhibitor, such as an anti-PD-1 antibody), may be administered in an amount in the range of about 100 μ/kg body weight to about 20 mg/kg body weight per dose. In some embodiments, a anticancer agent, such as checkpoint inhibitor (e.g. PD-1 inhibitor, such as an anti-PD-1 antibody), may be administered in an amount in the range of about 0.5 mg/kg body weight to about 20 mg/kg body weight per dose.C.

C. Subjects for Treatment

In some embodiments, the provided methods are for treating a subject that is or is suspected of having the disease or condition for which the therapeutic application is directed. In some cases, the subject for treatment can be selected prior to treatment based on one or more features or parameters, such as to determine suitability for the therapy or to identify or select subjects for treatment in accord with any of the provided embodiments, including treatment with any of the provided variant CD80 polypeptides or variant CD80 IgSF domain fusion proteins.

In some embodiments, provided methods include diagnostic, prognostic or monitoring methods utilizing binding assays on various biological samples of patients having a disease or condition in which is known, suspected or that may be a candidate for treatment in accord with the provided embodiments. In some embodiments, the methods are carried out with reagents capable of detecting one or more cells surface marker expressed, or likely to be expressed, on tumors or tumor cell infiltrates. In some aspects, the one or more cell markers include those in which tumors or tumor cell infiltrates express one or more binding partner (e.g. CD28, PD-L1 and/or CTLA-4) or competing cell surface ligand (e.g. CD80 or CD86) of the variant CD80 polypeptide to be utilized in the therapeutic methods. In some aspects, a reagent is employed that is able to detect a cell surface marker of T cells, such as tumor infiltrating T lymphocytes, e.g. a CD3 binding reagent. Such reagents can be used as companion diagnostics for selecting subjects that are most likely to benefit from treatment with the provided molecules or pharmaceutical compositions and/or for predicting efficacy of the treatment.

In some embodiments, methods are provided for selecting subjects and/or predicting efficacy of treatment with provided therapies based on activity of provided variant CD80 polypeptides or variant CD80 IgSF domain fusion proteins to antagonize or block CTLA-4, antagonize or block PD-L1/PD-1 interaction and/or to mediate CD28 agonism, such as PD-L1-dependent CD28 costimulation, including in methods for increasing an immune response for treating a disease or condition and/or for treating a tumor or cancer.

In some embodiments, the reagent is binding reagent that specifically binds to the cell surface marker (e.g. CD28, CD80 (B7-1), CD86 (B7-2) PD-L1, or CTLA-4) on the surface of a cell. In some embodiments, the binding reagent can be an antibody or antigen-binding fragment, protein ligand or binding partner, an aptamer, an affimer, a peptide or a hapten. In some embodiments, such reagents can be used as a companion diagnostic for selecting or identifying subjects for treatment with a therapeutic agent or pharmaceutical composition provided herein containing a variant CD80 polypeptide that is or contains an IgSF domain. Included among such therapeutic agents are fusion proteins containing an extracellular portion of a CD80 variant polypeptide containing an affinity modified IgSF domain (e.g. IgV) is linked, directly or indirectly, to a multimerization domain, e.g. an Fc domain or region. In some embodiments, such a therapeutic agent is a variant CD80-Fc fusion protein.

In some embodiments, prior to administering a provided pharmaceutical composition (including pharmaceutical composition comprising the variant CD80 IgSF domain fusion proteins) to a subject, such as a subject known or suspected of having a cancer, the method includes obtaining a biological sample from the subject for assessment of the presence or absence, or degree of presence, of a cell surface marker as described. In some embodiments, the provided methods including contacting a biological sample from a subject with a binding reagent (e.g. antibody) capable of specifically binding to the ectodomain of the cell surface marker (e.g. CD28, CD80 (B7-1), CD86 (B7-2, PD-L1, or CTLA-4) and detecting the presence or absence of the bound binding reagent in or on cells of the biological sample. In some embodiments, the biological sample is a tumor tissue sample comprising stromal cells, tumor cells or tumor infiltrating cells, such as tumor infiltrating immune cells, e.g. tumor infiltrating lymphocytes.

In some embodiments, it is desired to detect, in a subject suspected of having a cancer, cells that are surface negative for a cell surface marker that is, is likely or may be a competing cell surface ligand to the variant CD80 polypeptide. In some aspects, a competing cell surface ligand is a ligand that, if expressed on cells in or around the tumor, may or has the potential to compete for binding of the variant CD80 polypeptide to one or more of its binding partners, such as CD28. For example, CD80 and CD86 are cell surface markers that are expressed or may be expressed on antigen presenting cells (APCs) or on tumor cells and are cognate binding partners for CD28. In some embodiments, the provided methods are carried out with reagents that are capable of binding to CD80 or CD86. In some embodiments of the provided methods, a biological sample is detected as having cells surface negative for CD80 or CD86, or cells that are relatively surface negative for CD80 or CD86, if there is not detectectable expression of CD80 or CD86 (e.g. following contacting with the binding reagent and detection of bound binding reagent) on cells of the biological sample and/or in which CD80 or CD86 is expressed on less than or less than about 20% of cells of the biological sample and/or in which CD80 or CD86 surface expression on cells of the biological sample is scored or identified as having a low intensity of cell membrane staining (e.g. score of 0 or 1). In some embodiments of the provided methods, a biological sample is detected as having cells that are relatively surface negative for CD80 or CD86 if less than or less than about 20% of the cells of the biological sample are surface positive for CD80 or CD86, such as less than or less than about 10% of the cells, less than or less than about 5% of the cells, less than or less than about 2% of the cells or less than or less than about 1% of the cells. In some embodiments, if the biological sample is determined or assessed to comprise cells that are surface negative for expression of CD80 or CD86, or relatively surface negative for expression of CD80 or CD86, the subject is selected for treatment.

In some embodiments, the binding reagent is an antibody or an antigen binding fragment thereof that specifically binds CD80 (B7-1) or CD86 (B7-2). Various reagents, including antibodies, specific for CD80 or CD86, including human CD80 or human CD86, are known. Exemplary antibodies for use in diagnostics tests or as part of a kit for diagnostics is provided in Table 4.

TABLE 4
Exemplary Antibodies for Use in Diagnostics Tests

| Antibody | IgG Isotype | Supplier (Catalogue Number) |
|---|---|---|
| Anti-CD80 | Rabbit IgG | Abcam (ab134120) |
| [EPR1157(2)] | (monoclonal) | |
| Anti-CD80 [2D10] | Mouse IgG1k | BioLegend (305202) |
| Anti-CD80 [775] | Rabbit IgG | Sino Biologicals |
| | (monoclonal) | (10698-R775) |
| Anti-CD86 [BU63] | Mouse IgG1k | Abcam (ab234000) |
| Anti-CD86 [CDLA86] | Mouse IgG1k | Source Bioscience |
| | | (LS-C392134) |
| Anti-CD86 [118] | Rabbit IgG | Sino Biologicals |
| | (monoclonal) | (10699-R118) |
| Anti-CD86 [C86/2160R] | Rabbit IgG | Abcam (ab234401) |
| | (monoclonal) | |

In some embodiments, the provided methods include contacting a biological sample from a subject with an anti-CD80 antibody EPR1157(2) and detecting the presence or absence of the bound binding reagent in or on cells of the biological sample. In some embodiments, the provided methods include contacting a biological sample from a subject with an anti-CD80 antibody 2D10 and detecting the presence or absence of the bound binding reagent in or on cells of the biological sample. In some embodiments, the provided methods include contacting a biological sample from a subject with an anti-CD80 antibody 775 and detecting the presence or absence of the bound binding reagent in or on cells of the biological sample. In some embodiments, the provided methods include contacting a biological sample from a subject with an anti-CD86 antibody BU63 and detecting the presence or absence of the bound binding reagent in or on cells of the biological sample. In some embodiments, the provided methods include contacting a biological sample from a subject with an anti-CD86 antibody CDLA86 and detecting the presence or absence of the bound binding reagent in or on cells of the biological sample. In some embodiments, the provided methods include contacting a biological sample from a subject with an anti-CD86 antibody 118 and detecting the presence or absence of the bound binding reagent in or on cells of the biological sample. In some embodiments, the provided methods include contacting a biological sample from a subject with an anti-CD86 antibody C86/2160R and detecting the presence or absence of the bound binding reagent in or on cells of the biological sample. In some embodiments, the biological sample is a tumor tissue sample comprising stromal cells, tumor cells or tumor infiltrating cells, such as tumor infiltrating immune cells, e.g. tumor infiltrating lymphocytes.

In some embodiments, it is desired to detect, in a subject suspected of having a cancer, cells that are surface positive for a cell surface marker that is or comprises a binding partner of a variant CD80 polypeptide. In some aspects, the binding partner is cell surface CD28, PD-L1 or CTLA-4, which, in some cases, can be expressed on tumor infiltrating T cells, antigen presenting cells or tumor cells. In some embodiments, a biological sample is detected for cells surface positive for a cell surface marker, e.g. CD28, PD-L1, or CTLA-4, if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more.

In some embodiments, the tumor tissue sample is detected for cells surface positive for PD-L1 if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more. In some embodiments, the cells are tumor cells or tumor infiltrating immune cells. In some embodiments, the tumor tissue sample is detected for cells surface positive for CD28 if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more. In some embodiments, the cells are tumor infiltrating immune lymphocytes. In some embodiments, if the biological sample is determined or assessed to comprise cells that are surface positive for expression of PD-L1, or relatively surface positive for expression of PD-L1, the subject is selected for treatment.

In some embodiments, the reagent is a PD-L1-binding reagent that specifically binds to PD-L1 on the surface of a cell, such as on the surface of a tumor cell or myeloid cells present in the tumor environment. In some embodiments, the binding reagent is an antibody or an antigen binding fragment thereof that specifically binds PD-L1. Various companion diagnostic reagents for detecting PD-L1, such as human PD-L1, including intracellular or extracellular PD-L1, are known, e.g. Roach et al. (2016) Appl. Immunohistochem., Mol. Morphol., 24:392-397; Cogswell et al. (2017) Mol. Diagn. Ther. 21:85-93; International published patent application No. WO2015/181343 or WO2017/085307, or U.S. published patent application No. US2016/0009805 or US2017/0285037. Non limiting examples of anti-PD-L1 antibodies include, but are not limited to, mouse anti-PD-L1 clone 22C3 (Merck & Co.), rabbit anti-PD-L1 clone 28-8 (Bristol-Myers Squibb), rabbit anti-PD-L1 clones SP263 or SP142 (Spring Biosciences) and rabbit anti-PD-L1 antibody clone E1L3N. Such binding reagents can be used in histochemistry methods, including those available as Dako PD-L1 IHC 22C3 pharmDx assay, PD-L1 IHC 28-8 pharmDx assay, Ventana PD-L1 (SP263) assay, or Ventana PD-L1 (SP142) assay.

In some embodiments, the tumor tissue sample is detected for cells surface positive for CD28 if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more. In some embodiments, the cells are tumor infiltrating immune cells, such as tumor infiltrating T lymphocytes. In some embodiments, if the biological sample is determined or assessed to comprise cells that are surface positive for expression of CD28, or relatively surface positive for expression of CD28, the subject is selected for treatment. In some embodiments, the binding reagent is an antibody or an antigen-binding fragment thereof that specifically binds CD28. Various reagents, including antibodies, specific for CD28, including human CD28, are known. Non-limiting examples of anti-CD28 antibodies include, but are not limited to, anti-CD28 antibody 007 (Sino Biologicals, 11524-R007) or anti-CD28 antibody C28/77 (NovusBio, NBO2-32817).

In some embodiments, the tumor tissue sample is detected for cells surface positive for CTLA-4 if there is a detectable expression level of the binding partner (e.g. following contacting with the binding reagent and detection of bound binding reagent) in at least or at least about or about 1% of the cells, at least or at least about or about 5% of the cells, at least or at least about or about 10% of the cells, at least or at least about or about 20% of the cells, at least or at least about or about 40% of the cells or more. In some embodiments, the cells are tumor infiltrating immune cells, such as tumor infiltrating T lymphocytes. In some embodiments, if the biological sample is determined or assessed to comprise cells that are surface positive for expression of CTLA-4, or relatively surface positive for expression of CTLA-4, the subject is selected for treatment. In some embodiments, the binding reagent is an antibody or an antigen-binding fragment thereof that specifically binds CTLA-4. Various reagents, including antibodies, specific for CTLA-4, including human CTLA-4, are known.

In some embodiments, the methods further can include methods for scoring the immune response in a subject with a cancer or suspected of having a cancer, such as using Immunoscore or similar methods for assessing immune cell infiltrates. In some aspects, such methods include methods for identifying or evaluating specific lymphocyte populations, such as T cells. For example, an immunoscore includes a quantifiable measure of a tumor-infiltrating lymphocytes. In some cases, the methods involve the use of a binding reagent that is capable of binding to CD3, which is generally a universal marker for T cells. In some aspects, further analysis may be done to identify the type of T cells, e.g. regulatory or cytototic T cells, such as based on CD45RO, CD8 or other marker of a T cell subset or type. In some cases, an immunoscore is based on the density of two lymphocyte populations, cytotoxic (CD8) and memory (CD45RO) T cells. Other immunoscore-like markers can be employed. In some cases, aspects of scoring or assessing an immune response, such as by analyzing the presence or absence of T lymphocytes, can be carried out using multiplex methods. Exemplary methods for analyzing or assessing an immune response in a subject, such as for analyzing the presence or absence of certain T lymphocyte populations in a biological sample in a subject are known, see e.g. Galon et al. (2012) Journal of Translational Medicine, 10:1; Galon et al. (2006) Science, 313:1960-1964; Galon et al. (2016) Journal of Translational Medicine, 14:273; Ascierto et al. (2013) Journal of Translational Medicine, 11:54; Kwak et al. (2016) Oncotarget, 7:81778-81790; U.S. patent application publication US20160363593. Further, any of the provided methods described herein for assessing or detecting a surface marker as described can be multiplexed together, including in methods for also assessing or scoring for the presence or absence of an immune response or presence of absence of T lymphocytes.

The binding reagent can be conjugated, such as fused, directly or indirectly to a detectable label for detection. In some cases, the binding reagent is linked or attached to a moiety that permits either direct detection or detection via secondary agents, such as via antibodies that bind to the reagent or a portion of the reagent and that are coupled to a detectable label. Exemplary detectable labels include, for example, chemiluminescent moieties, bioluminescent moieties, fluorescent moieties, radionuclides, and metals. Methods for detecting labels are well known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Exemplary detectable labels include, for example, chemiluminescent moieties, bioluminescent moieties, fluorescent moieties, radionuclides, and metals. Among detectable labels are fluorescent probes or detectable enzymes, e.g. horseradish perioxidase.

The binding reagents can detect the cell surface marker, e.g. CD28, CD80 (B7-1), CD86 (B7-2) PD-L1, or CTLA-4, using any binding assay known to one of skill in the art including, in vitro or in vivo assays. Exemplary binding assays that can be used to assess, evaluate, determine, quantify and/or otherwise specifically detect expression or levels of a cell surface marker in a sample include, but are not limited to, solid phase binding assays (e.g. enzyme linked immunosorbent assay (ELISA)), radioimmunoassay (RIA), immunoradiometric assay, fluorescence assay, chemiluminescent assay, bioluminescent assay, western blot and histochemistry methods, such as immunohistochemistry (IHC) or pseudo immunohistochemistry using a non-antibody binding agent. In solid phase binding assay methods, such as ELISA methods, for example, the assay can be a sandwich format or a competitive inhibition format. In other examples, in vivo imaging methods can be used. The binding assay can be performed on samples obtained from a patient body fluid, cell or tissue sample of any type, including from plasma, urine, tumor or suspected tumor tissues (including fresh, frozen, and fixed or paraffin embedded tissue), lymph node or bone marrow. In exemplary methods to select a subject for treatment in accord with the therapeutic methods provided herein, harvesting of the sample, e.g. tumor tissue, is carried out prior to treatment of the subject.

In some embodiments, the binding assay is a tissue staining assay to detect the expression or levels of a binding partner in a tissue or cell sample. Tissue staining methods include, but are not limited to, cytochemical or histochemical methods, such as immunohistochemistry (IHC) or histochemistry using a non-antibody binding agent (e.g. pseudo immunohistochemistry). Such histochemical methods permit quantitative or semi-quantitative detection of the amount of the binding partner in a sample, such as a tumor tissue sample. In such methods, a tissue sample can be contacted with a binding reagent, and in particular one that is detectably labeled or capable of detection, under conditions that permit binding to a tissue- or cell-associated cell surface marker as described.

A sample for use in the methods provided herein as determined by histochemistry can be any biological sample that is associated with the disease or condition, such as a tissue or cellular sample. For example, a tissue sample can be solid tissue, including a fresh, frozen and/or preserved organ or tissue sample or biopsy or aspirate, or cells. In some examples, the tissue sample is tissue or cells obtained from a solid tumor, such as primary and metastatic tumors, including but not limited to, breast, colon, rectum, lung, stomach, ovary, cervix, uterus, testes, bladder, prostate, thyroid and lung cancer tumors. In particular examples, the sample is a tissue sample from a cancer that is a late-stage cancer, a metastatic cancer, undifferentiated cancer, ovarian cancer, in situ carcinoma (ISC), squamous cell carcinoma (SCC), prostate cancer, pancreatic cancer, non-small cell lung cancer, breast cancer, colon cancer.

In some aspects, when the tumor is a solid tumor, isolation of tumor cells can be achieved by surgical biopsy. Biopsy techniques that can be used to harvest tumor cells from a subject include, but are not limited to, needle biopsy, CT-guided needle biopsy, aspiration biopsy, endoscopic biopsy, bronchoscopic biopsy, bronchial lavage, incisional biopsy, excisional biopsy, punch biopsy, shave biopsy, skin biopsy, bone marrow biopsy, and the Loop Electrosurgical Excision Procedure (LEEP). Typically, a non-necrotic, sterile biopsy or specimen is obtained that is greater than 100 mg, but which can be smaller, such as less than 100 mg, 50 mg or less, 10 mg or less or 5 mg or less; or larger, such as more than 100 mg, 200 mg or more, or 500 mg or more, 1 gm or more, 2 gm or more, 3 gm or more, 4 gm or more or 5 gm or more. The sample size to be extracted for the assay can depend on a number of factors including, but not limited to, the number of assays to be performed, the health of the tissue sample, the type of cancer, and the condition of the subject. The tumor tissue is placed in a sterile vessel, such as a sterile tube or culture plate, and can be optionally immersed in an appropriate medium.

In some embodiments, tissue obtained from the patient after biopsy is fixed, such as by formalin (formaldehyde) or glutaraldehyde, for example, or by alcohol immersion. For histochemical methods, the tumor sample can be processed using known techniques, such as dehydration and embedding the tumor tissue in a paraffin wax or other solid supports known to those of skill in the art (see Plenat et ah, (2001) Ann Pathol. January 21(1):29-47), slicing the tissue into sections suitable for staining, and processing the sections for staining according to the histochemical staining method selected, including removal of solid supports for embedding by organic solvents, for example, and rehydration of preserved tissue.

In some embodiments, histochemistry methods are employed. In some cases, the binding reagent is directly attached or linked to a detectable label or other moiety for direct or indirect detection. Exemplary detectable regents including, but are not limited to, biotin, a fluorescent protein, bioluminescent protein or enzyme. In other examples, the binding reagents are conjugated, e.g. fused, to peptides or proteins that can be detected via a labeled binding partner or antibody. In some examples, a binding partner can be detected by HC methods using a labeled secondary reagent, such as labeled antibodies, that recognize one or more regions, e.g. epitopes, of the binding reagent.

In some embodiments, the resulting stained specimens, such as obtained by histochemistry methods, are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the cell surface marker, e.g. e.g. CD28, CD80 (B7-1), CD86 (B7-2) PD-L1, or CTLA-4, in the sample. Various automated sample processing, scanning and analysis systems suitable for use with immunohistochemistry are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g. published U.S. patent Appl. No. US20100136549).

In some embodiments, the diagnostic tests are used prior to, during, and/or after treatment containing the provided variant CD80 polypeptides. In some embodiments, the provided diagnostic tests predict the likelihood and/or degree of a subject having a response to a treatment containing the provided variant CD80 polypeptides. Also provided are methods for selecting a therapy for a subject with a disease or condition that is a tumor or cancer.

### V. KITS AND ARTICLES OF MANUFACTURE

Also provided herein are articles of manufacture that comprise the pharmaceutical compositions described herein (including pharmaceutical composition comprising the variant CD80 IgSF domain fusion proteins) in suitable packaging. Among suitable packaging for articles of manufacture include one or more containers, typically a plurality of containers, packaging material, and a label or package insert on or associated with the container or containers and/or packaging, generally including instructions for administration of the composition to a subject. Suitable containers for packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

The article of manufacture may further include a package insert or label with one or more pieces of identifying information and/or instructions for use. In some embodiments, the information or instructions indicates that the contents can or should be used to treat a particular condition or disease, and/or providing instructions therefor. The label or package insert may indicate that the contents of the article of manufacture are to be used for treating the disease or condition. In some embodiments, the label or package insert provides instructions to treat a subject, e.g., according to any of the embodiments of the provided methods. In some embodiments, the instructions specify administering one or more of the unit doses to the subject.

Further provided are kits comprising the pharmaceutical compositions (or articles of manufacture) described herein, which may further comprise instruction(s) on methods of using the composition, such as uses described herein. The kits described herein may also include other materials desirable from a commercial and user standpoint, including other buffers, diluents, filters, needles, syringes, and package inserts with instructions for performing any methods described herein.

### VI. EXEMPLARY EMBODIMENTS

Among the provided embodiments are:

1. A method of treating a cancer in a subject, the method comprising:

(a) administering to a subject having a cancer a variant CD80 fusion protein that specifically binds to PD-L1, said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide; and

(b) administering to the subject a therapeutically effective amount of a PD-1 inhibitor, wherein the PD-1 inhibitor disrupts the interaction between Programmed Death-1 (PD-1) and a ligand thereof.

2. The method of embodiment 1 wherein the ligand is Programmed Death Ligand-1 (PD-L1) or PD-L2.

3. The method of embodiment 1 or embodiment 2, wherein the PD-1 inhibitor specifically binds to PD-1.

4. The method of embodiment 1 or embodiment 2, wherein the PD-1 inhibitor does not compete with the variant CD80 fusion protein for binding to PD-L1.

5. The method of any of embodiments 1-4, wherein the PD-1 inhibitor is a peptide, protein, antibody or antigen-binding fragment thereof, or a small molecule.

6. The method of any of embodiments 1-5, wherein the PD-1 inhibitor is an antibody or antigen-binding fragment thereof that specifically binds to PD-1.

7. The method of any of embodiments 1-6, wherein the antibody or antigen-binding portion is selected from nivolumab, pembrolizumab, MEDI0680 (AMP514), PDR001, cemiplimab (REGN2810), pidilizumab (CT011), or an antigen-binding portion thereof.

8. The method of any of embodiments 1-7, wherein the PD-1 inhibitor comprises the extracellular domain of PD-L2 or a portion thereof that binds to PD-1, and an Fc region.

9. The method of embodiment 8, wherein the PD-1 inhibitor is AMP-224.

10. The method of any of embodiments 1-9, wherein the initiation of the administration of the PD-1 inhibitor is carried out concurrently or sequentially with the initiation of the administration of the variant CD80 fusion protein.

11. The method of any of embodiments 1-10, wherein the initiation of the administration of the PD-1 inhibitor is after the initiation of the administration of the variant CD80 fusion protein.

12. The method of any of embodiments 1-11, wherein the initiation of the administration of the anti-PD-1 antibody is after the administration of the last dose of a therapeutically effective amount of the variant CD80 fusion protein.

13. The method of any of embodiments 1-12, wherein the variant CD80 fusion protein is administered in a therapeutically effective amount as a single dose or in six or fewer multiple doses.

14. A method of treating a cancer in a subject, the method comprising administering to a subject having a cancer a therapeutically effective amount of a variant CD80 fusion protein, said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide, wherein the therapeutically effective amount of the variant CD80 fusion protein is administered as a single dose or in six or fewer multiple doses.

15. The method of any of embodiments 1-14, wherein the variant CD80 fusion protein is administered parenterally.

16. The method of any of embodiments 1-15, wherein the variant CD80 fusion protein is administered subcutaneously.

17. The method of any of embodiments 1-15, wherein the variant CD80 fusion protein is administered intravenously.

18. The method of any of embodiments 1-17, wherein the variant CD80 fusion protein is administered by injection that is a bolus injection.

19. The method of any of embodiments 13-18, wherein the therapeutically effective amount is between about 0.5 mg/kg and about 140 mg/kg, about 0.5 mg/kg and about 30 mg/kg, about 0.5 mg/kg and about 20 mg/kg, about 0.5 mg/kg and about 18 mg/kg, about 0.5 mg/kg and about 12 mg/kg, about 0.5 mg/kg and about 10 mg/kg, about 0.5 mg/kg and about 6 mg/kg, about 0.5 mg/kg and about 3 mg/kg, about 1 mg/kg and about 40 mg/kg, about 1 mg/kg and about 30 mg/kg, about 1 mg/kg and about 20 mg/kg, about 1 mg/kg and about 18 mg/kg, about 1 mg/kg and about 12 mg/kg, about 1 mg/kg and about 10 mg/kg, about 1 mg/kg and about 6 mg/kg, about 1 mg/kg and about 3 mg/kg, about 3 mg/kg and about 40 mg/kg, about 3 mg/kg and about 30 mg/kg, about 3 mg/kg and about 20 mg/kg, about 3 mg/kg and about 18 mg/kg, about 3 mg/kg and about 12 mg/kg, about 3 mg/kg and about 10 mg/kg, about 3 mg/kg and about 6 mg/kg, about 6 mg/kg and about 40 mg/kg, about 6 mg/kg and about 30 mg/kg, about 6 mg/kg and about 20 mg/kg, about 6 mg/kg and about 18 mg/kg, about 6 mg/kg and about 12 mg/kg, about 6 mg/kg and about 10 mg/kg, about 10 mg/kg and about 40 mg/kg, about 10 mg/kg and about 30 mg/kg, about 10 mg/kg and about 20 mg/kg, about 10 mg/kg and about 18 mg/kg, about 10 mg/kg and about 12 mg/kg, about 12 mg/kg and about 40 mg/kg, about 12 mg/kg and about 30 mg/kg, about 12 mg/kg and about 20 mg/kg, about 12 mg/kg and about 18 mg/kg, about 18 mg/kg and about 40 mg/kg, about 18 mg/kg and about 30 mg/kg, about 18 mg/kg and about 20 mg/kg, about 20 mg/kg and about 40 mg/kg, about 20 mg/kg and about 30 mg/kg or about 30 mg/kg and about 40 mg/kg, each inclusive.

20. The method of any of embodiments 13-19, wherein the therapeutically effective amount is between about 3.0 mg/kg and 18 mg/kg, inclusive.

21. The method of any of embodiments 13-19, wherein the therapeutically effective amount is between about 6 mg/kg and about 20 mg/kg, inclusive.

22. The method of any of embodiment 13-19, wherein the therapeutically effective amount is between about 1 mg/kg and about 10 mg/kg, inclusive.

23. The method of any of embodiments 13-19 and 22, wherein the therapeutically effective amount is between about 2.0 mg/kg and about 6.0 mg/kg, inclusive.

24. The method of any of embodiments 1-23, wherein the variant CD80 fusion protein is administered intratumorally.

25. A method of treating a cancer in a subject, the method comprising intratumorally administering to a subject having a cancer a therapeutically effective amount of a variant CD80 fusion protein, said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide.

26. The method of embodiment 25, wherein the variant CD80 fusion protein is administered in a therapeutically effective amount as a single dose or in six or fewer multiple doses.

27. The method of any of embodiments 1-18 and 24-26, wherein the therapeutically effective amount is between about 0.1 mg/kg and about 1 mg/kg, inclusive.

28. The method of any of embodiments 1-18 and 24-27, wherein the therapeutically effective amount is between about 0.2 mg/kg and about 0.6 mg/kg.

29. The method of any of embodiments 13-24 and 26-28, wherein the therapeutically effective amount is administered in a single dose.

30. The method of any of embodiments 13-24 and 26-28, wherein the therapeutically effective amount is administered in six or fewer multiple doses and the six or fewer multiple doses is two doses, three doses, four doses, five doses or six doses.

31. The method of embodiment 30, wherein the therapeutically effective amount is administered in four doses.

32. The method of embodiment 30, wherein the therapeutically effective amount is administered in three doses.

33. The method of embodiment 30, wherein the therapeutically effective amount is administered in two doses.

34. The method of any of embodiments 30-33, wherein each of the six or fewer multiple doses is administered weekly, every two weeks, every three weeks or every four weeks.

35. The method of any of embodiments 30-33, wherein the interval between each multiple dose is about a week.

36. The methods of any of embodiments 13-19 and 29-35 wherein the single dose or each of the six or fewer multiple doses, individually, is administered in an amount between about 0.5 mg/kg and about 10 mg/kg once every week (Q1W).

37. A method of treating a cancer in a subject, the method comprising administering to a subject having a cancer a variant CD80 fusion protein in an amount of between about 1.0 mg/kg to 10 mg/kg, inclusive, once every week (Q1W), wherein said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide, wherein the variant CD80 fusion protein is administered.

38. The method of embodiment 36 or 37, wherein the amount of the variant CD80 fusion protein administered Q1W is between about 1 mg/kg and about 3 mg/kg.

39. The method of embodiment 36-38, wherein the administration is for more than one week.

40. The methods of any of embodiments 13-19, 29-34, wherein the single dose or six or fewer multiple doses, individually, is administered in an amount between about 1.0 mg/kg and about 40 mg/kg once every three weeks (Q3W).

41. A method of treating a cancer in a subject, the method comprising administering to a subject having a cancer a variant CD80 fusion protein in an amount of between about 1.0 mg/kg to 40 mg/kg, inclusive, once every three weeks (Q3W), wherein said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide.

42. The method of embodiments 39 or embodiment 40, wherein the amount of the variant CD80 fusion protein administered Q3W is between about 3.0 mg/kg and about 10 mg/kg.

43. The method of any of embodiments 37-39, 41 and 42, wherein the variant CD80 fusion protein is administered parenterally, optionally subcutaneously.

44. The method of any of embodiments 37-39, 41-43, wherein the variant CD80 fusion protein is administered by injection that is a bolus injection.

45. The method of any of embodiments 13-44, wherein the therapeutically effective amount is administered in a time period of no more than six weeks.

46. The method of any of embodiments 13-44, wherein the therapeutically effective amount is administered in a time period of no more than four weeks or about four weeks.

47. The method of any of embodiments 13-44, wherein each multiple dose is an equal amount.

48. The method of any of embodiments 1-47, wherein prior to the administering, selecting a subject for treatment that has a tumor comprising cells surface positive for PD-L1 or CD28 and/or surface negative for a cell surface ligand selected from CD80 or CD86.

49. A method of treating a cancer in a subject, the method comprising administering a variant CD80 fusion protein to a subject selected as having a tumor comprising cells surface negative for a cell surface ligand selected from CD80 or CD86, and/or surface positive for CD28, wherein the variant CD80 fusion protein comprises a variant CD80 extracellular domain or a portion thereof comprising an IgV domain or a specific binding fragment thereof and a multimerization domain, said variant CD80 extracellular domain or the portion thereof comprising one or more amino acid modifications at one or more positions in the sequence of amino acids of the extracellular domain or a portion thereof of an unmodified CD80 polypeptide.

50. The method of embodiment 48 or embodiment 49, wherein the cells surface negative for CD80 or CD86 comprise tumor cells or antigen presenting cells.

51. The method of embodiment 48 or embodiment 49, wherein the cells surface positive for CD28 comprise tumor infiltrating T lymphocytes.

52. The method of any of embodiments 48-51, wherein the subject has further been selected as having a tumor comprising cells surface positive for PD-L1.

53. The method of embodiment 48 or embodiment 52, wherein the cells surface positive for PD-L1 are tumor cells or tumor infiltrating immune cells, optionally tumor infiltrating T lymphocytes.

54. The method of any of embodiments 48-53, further comprising determining an immunoscore based on the presence or density of tumor infiltrating T lymphocytes in the tumor of the subject.

55. The method of embodiment 54, wherein the subject is selected for treatment if the immunoscore is low.

56. The method of any of embodiments 48-55, wherein a subject is selected by immunohistochemistry (IHC) using a reagent that specifically binds to the at least one binding partner.

57. The method of any of embodiments 14-56, wherein the variant CD80 fusion protein exhibits increased binding to at least one binding partner selected from among CD28, PD-L1 and CTLA-4 compared to a fusion protein comprising the extracellular domain of the unmodified CD80 for the at least one binding partner.

58. The method of any of embodiments 14-57, wherein the variant CD80 fusion protein exhibits increased binding to PD-L1 compared to a fusion protein comprising the extracellular domain of the unmodified CD80 for the binding partner.

59. The method of any of embodiments 1-13, wherein the variant CD80 fusion protein further exhibits increased binding to at least one binding partner selected from among CD28 and CTLA-4 compared to a fusion protein comprising the extracellular domain of the unmodified CD80 for the at least one binding partner.

60. The method of any of embodiments 1-59, wherein the binding affinity is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 80-fold, 100-fold, 150-fold, 200-fold, 250-fold, 300-fold, 400-fold, or 450-fold compared to binding affinity of the unmodified CD80 for the ectodomain of the binding partner.

61. The method of any of embodiments 1-60, wherein the one or more amino acid modifications are amino acid substitutions.

62. The method of any of embodiments 1-61, wherein the one or more amino acid modifications comprise one or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

63. The method of any of embodiments 1-62, wherein the one or more amino acid modifications comprise two or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

64. The method of any of embodiments 1-63, wherein the one or more amino acid modifications comprises amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2.

65. The method of any of embodiments 1-64, wherein the one or more amino acid modifications comprise amino acid substitutions E35D/M47L/V68M, E35D/M47V/V68M or E35D/M47I/L70M.

66. The method of any of embodiments 1-65, wherein the one or more amino acid modifications comprise amino acid substitutions E35D/M47V/N48K/V68M/K89N.

67. The method of any of embodiments 1-65, wherein the one or more amino acid modifications comprise amino acid substitutions H18Y/A26E/E35D/M47L/V68M/A71G/D90G.

68. The method of any of embodiments 1-65, wherein the one or more amino acid modifications comprise amino acid substitutions E35D/D46E/M47V/V68M/D90G/K93E.

69. The method of any of embodiments 1-65, wherein the one or more amino acid modifications comprise amino acid substitutions E35D/D46V/M47L/V68M/L85Q/E88D.

70. The method of any of embodiments 1-69, wherein the unmodified CD80 is a human CD80.

71. The method of any of embodiments 1-70, wherein the extracellular domain or portion thereof of the unmodified CD80 comprises (i) the sequence of amino acids set forth in SEQ ID NO:2, (ii) a sequence of amino acids that has at least 95% sequence identity to SEQ ID NO:2; or (iii) is a portion of (i) or (ii) comprising an IgV domain or a specific binding fragment thereof.

72. The method of embodiment 71, wherein the extracellular domain or portion thereof of the unmodified CD80 is an extracellular domain portion that is or comprises the IgV domain or a specific binding fragment thereof.

73. The method of embodiment 72, wherein the extracellular domain portion of the unmodified CD80 comprises the IgV domain but does not comprise the IgC domain or a portion of the IgC domain.

74. The method of embodiment 72 or embodiment 73, wherein the extracellular domain portion of the unmodified CD80 is set forth as the sequence of amino acids 35-135 of SEQ ID NO:2 (SEQ ID NO:76) or 35-141 of SEQ ID NO:2 (SEQ ID NO:150).

75. The method of any of embodiments 1-74, wherein the variant CD80 extracellular domain or portion thereof is an extracellular domain portion that does not comprise the IgC domain or a portion of the IgC domain.

76. The method of any of embodiments 1-75, wherein the variant CD80 extracellular domain comprises the sequence of amino acids 35-135 of SEQ ID NO:2 (SEQ ID NO:76) or 35-141 of SEQ ID NO:2 (SEQ ID NO:150) in which is contained the one or more amino acid substitutions.

77. The method of any of embodiments 1-75, wherein the variant CD80 extracellular domain is the sequence of amino acids 35-135 of SEQ ID NO:2 (SEQ ID NO:76) or 35-141 of SEQ ID NO:2 (SEQ ID NO:150) in which is contained the one or more amino acid substitutions.

78. The method of any of embodiments 1-77, wherein the variant CD80 extracellular domain 1, 2, 3, 4, 5, 6, 7, 8, 9, 10, 11, 12, 13, 14, 15, 16, 17, 18, 19 or 20 amino acid modifications, optionally wherein the amino acid modifications are amino acid substitutions.

79. The method of any of embodiments 1-78, wherein the variant CD80 extracellular domain comprises no more than 1, 2, 3, 4, 5, 6, 7, 8, 9, 10, 11, 12 or 13 amino acid modifications, optionally wherein the amino acid modifications are amino acid substitutions.

80. The method of any of embodiments 1-79, wherein the amino acid sequence of the variant CD80 extracellular domain has at least or at least about 85%, 86%, 87%, 88%, 89%, 90%, 91%, 92%, 93%, 94%, 95%, 96%, 97%, 98%, 99% or more sequence identity to the sequence of amino acids 35-135 of SEQ ID NO:2 (SEQ ID NO:76) or 35-141 of SEQ ID NO:2 (SEQ ID NO:150).

81. The method of any of embodiments 1-80, wherein the multimerization domain is an Fc region.

82. The method of embodiment 81, wherein the Fc region is of an immunoglobulin G1 (IgG1) or an immunoglobulin G2 (IgG2) protein.

83. The method of embodiment 81 or embodiment 82, wherein the Fc region exhibits one or more effector functions.

84. The method of embodiment 81 or embodiment 82, wherein the Fc region is a variant Fc region comprising one or more amino acid substitutions in a wildtype Fc region, said variant Fc region exhibiting one or more effector function that is reduced compared to the wildtype Fc region, optionally wherein the wildtype human Fc is of human IgG1.

85. The method of embodiment 84, wherein the Fc region comprises the amino acid substitution N297G, wherein the residue is numbered according to the EU index of Kabat.

86. The method of embodiment 84, wherein the Fc region comprises the amino acid substitutions R292C/N297G/V302C, wherein the residue is numbered according to the EU index of Kabat.

87. The method of embodiment 84, wherein the Fc region comprises the amino acid substitutions L234A/L235E/G237A, wherein the residue is numbered according to the EU index of Kabat.

88. The method of any of embodiments 81-87, wherein the Fc region further comprises the amino acid substitution C220S, wherein the residues are numbered according to the EU index of Kabat.

89. The method of any of embodiments 81-88, wherein the Fc region comprises K447del, wherein the residue is numbered according to the EU index of Kabat.

90. The method of any of embodiments 14-89, wherein the variant CD80 fusion protein antagonizes the activity of CTLA-4.

91. The method of any of embodiments 14-90, wherein the variant CD80 fusion protein blocks the PD-1/PD-L1 interaction.

92. The method of any of embodiments 14-91, wherein the variant CD80 fusion proteins binds to CD28 and mediates CD28 agonism.

93. The method of embodiment 92, wherein the CD28 agonism is PD-L1 dependent.

94. The method of any of embodiments 1-93, wherein the subject is a human.

95. A kit, comprising:

(a) a variant CD80 fusion protein that specifically binds to PD-L1, said variant CD80 fusion protein comprising a variant CD80 extracellular domain or a portion thereof comprising an IgV domain or a specific binding fragment thereof and a multimerization domain, wherein the variant CD80 extracellular domain or the portion thereof comprises one or more amino acid modifications at one or more positions in the sequence of amino acids of the extracellular domain or a portion thereof of an unmodified CD80 polypeptide; and

(b) a PD-1 inhibitor, wherein the PD-1 inhibitor disrupts the interaction between Programmed Death-1 (PD-1) and a ligand thereof.

96. The kit of embodiment 95, wherein the ligand is Programmed Death Ligand-1 (PD-L1) or PD-L2.

97. The kit of embodiment 95 or embodiment 96, wherein the PD-1 inhibitor specifically binds to PD-1.

98. The kit of any of embodiments 95-97, wherein the PD-1 inhibitor does not compete with the variant CD80 fusion protein for binding to PD-L1.

99. The kit of embodiment 95, wherein the PD-1 inhibitor is a peptide, protein, antibody or antigen-binding fragment thereof, or a small molecule.

100. The kit of embodiment 95-99, wherein the PD-1 inhibitor is an antibody or antigen-binding fragment thereof that specifically binds to PD-1.

101. The kit of embodiment 100, wherein the antibody or antigen-binding portion is selected from nivolumab, pembrolizumab, MEDI0680 (AMP514), PDR001, cemiplimab (REGN2810), pidilizumab (CT011), or an antigen-binding portion thereof.

102. The kit of any of embodiments 95-99, wherein the PD-1 inhibitor comprises the extracellular domain of PD-L2 or a portion thereof that binds to PD-1, and an Fc region.

103. The kit of embodiment 102, wherein the PD-1 inhibitor is AMP-224.

104. The kit of any of embodiments 95-103, wherein the variant CD80 fusion protein further exhibits increased binding to at least one binding partner selected from among CD28 and CTLA-4 compared to a fusion protein comprising the extracellular domain of the unmodified CD80 for the at least one binding partner.

105. The kit of any of embodiments 95-104, wherein the binding affinity is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 80-fold, 100-fold, 150-fold, 200-fold, 250-fold, 300-fold, 400-fold, or 450-fold compared to binding affinity of the unmodified CD80 for the ectodomain of the binding partner.

106. The kit of any of embodiments 95-105, wherein the one or more amino acid modifications are amino acid substitutions.

107. The kit of any of embodiments 95-106, wherein the one or more amino acid modifications comprise one or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

108. The kit of any of embodiments 95-107, wherein the one or more amino acid modifications comprise two or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

109. The kit of any of embodiments 95-108, wherein the one or more amino acid modifications comprises amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2.

110. The kit of any of embodiments 95-109, wherein the one or more amino acid modifications comprise amino acid substitutions E35D/M47L/V68M, E35D/M47V/V68M or E35D/M47I/L70M.

111. The kit of any of embodiments 95-110, wherein the one or more amino acid modifications comprise amino acid substitutions E35D/M47V/N48K/V68M/K89N, H18Y/A26E/E35D/M47L/V68M/A71G/D90G, E35D/D46E/M47V/V68M/D90G/K93E or E35D/D46V/M47L/V68M/L85Q/E88D.

112. The kit of any of embodiments 95-111, wherein the unmodified CD80 is a human CD80.

113. The kit of any of embodiments 95-112, wherein the extracellular domain or portion thereof of the unmodified CD80 comprises (i) the sequence of amino acids set forth in SEQ ID NO:2, (ii) a sequence of amino acids that has at least 95% sequence identity to SEQ ID NO:2; or (iii) is a portion of (i) or (ii) comprising an IgV domain or a specific binding fragment thereof.

114. The kit of embodiment 113, wherein the extracellular domain or portion thereof of the unmodified CD80 is an extracellular domain portion that is or comprises the IgV domain or a specific binding fragment thereof.

115. The kit of embodiment 114, wherein the extracellular domain portion of the unmodified CD80 comprises the IgV domain but does not comprise the IgC domain or a portion of the IgC domain.

116. The kit of embodiment 114 or embodiment 115, wherein the extracellular domain portion of the unmodified CD80 is set forth as the sequence of amino acids 35-135 of SEQ ID NO:2 (SEQ ID NO:76) or 35-141 of SEQ ID NO:2 (SEQ ID NO:150).

117. The kit of any of embodiments 95-116, wherein the variant CD80 extracellular domain or portion thereof is an extracellular domain portion that does not comprise the IgC domain or a portion of the IgC domain.

118. The kit of any of embodiments 95-117, wherein the variant CD80 extracellular domain comprises the sequence of amino acids 35-135 of SEQ ID NO:2 (SEQ ID NO:76) or 35-141 of SEQ ID NO:2 (SEQ ID NO:150) in which is contained the one or more amino acid substitutions.

119. The kit of any of embodiments 95-118, wherein the variant CD80 extracellular domain is the sequence of amino acids 35-135 of SEQ ID NO:2 (SEQ ID NO:76) or 35-141 of SEQ ID NO:2 (SEQ ID NO:150) in which is contained the one or more amino acid substitutions.

120. The kit of any of embodiments 95-119, wherein the variant CD80 extracellular domain 1, 2, 3, 4, 5, 6, 7, 8, 9, 10, 11, 12, 13, 14, 15, 16, 17, 18, 19 or 20 amino acid modifications, optionally wherein the amino acid modifications are amino acid substitutions.

121. The kit of any of embodiments 95-120, wherein the variant CD80 extracellular domain comprises no more than 1, 2, 3, 4, 5, 6, 7, 8, 9, 10, 11, 12 or 13 amino acid modifications, optionally wherein the amino acid modifications are amino acid substitutions.

122. The kit of any of embodiments 95-121, wherein the variant CD80 extracellular domain has at least or at least about 85%, 86%, 87%, 88%, 89%, 90%, 91%, 92%, 93%, 94%, 95%, 96%, 97%, 98%, 99% or more sequence identity to the sequence of amino acids 35-135 of SEQ ID NO:2 (SEQ ID NO:76) or 35-141 of SEQ ID NO:2 (SEQ ID NO:150).

123. The kit of any of embodiments 1-122, wherein the multimerization domain is an Fc region.

124. The kit of embodiment 123, wherein the Fc region is of an immunoglobulin G1 (IgG1) or an immunoglobulin G2 (IgG2) protein.

125. The kit of embodiment 123 or embodiment 124, wherein the Fc region exhibits one or more effector functions.

126. The kit of any of embodiments 123-125, wherein the Fc region is a variant Fc region comprising one or more amino acid substitutions in a wildtype Fc region, said variant Fc region exhibiting one or more effector function that is reduced compared to the wildtype Fc region, optionally wherein the wildtype human Fc is of human IgG1.

127. An article of manufacture comprising the kit of any of embodiments 95-126 and instructions for use.

128. The article of manufacture of embodiment 127, wherein the instructions provide information for administration of the variant CD80 Fc fusion protein or PD-1 inhibitor in accord with the methods 1-13, 19-24 and 27-94.

129. A multivalent CD80 polypeptide comprising two copies of a fusion protein comprising: (1) at least two variant CD80 extracellular domains or a portion thereof comprising an IgV domain or a specific binding fragment thereof (vCD80), wherein the vCD80 comprises one or more amino acid modifications at one or more positions in the sequence of amino acids of the extracellular domain or a portion thereof of an unmodified CD80 polypeptide and (2) an Fc polypeptide.

130. The multivalent CD80 polypeptide of embodiment 129, wherein the polypeptide is tetravalent.

131. The multivalent CD80 polypeptide of embodiment 129 or embodiment 130, wherein the fusion protein comprises the structure: (vCD80)-Linker-Fc-Linker-(vCD80).

132. The multivalent CD80 polypeptide of embodiment 129 or embodiment 130, wherein the fusion protein comprises the structure: (vCD80)-Linker-(vCD80)-Linker-Fc.

133. The multivalent CD80 polypeptide of embodiment 132, wherein the vCD80 exhibits increased binding to at least one binding partner selected from among CD28, PD-L1 and CTLA-4 compared to a vCD80 comprising the extracellular domain of the unmodified CD80 for the at least one binding partner.

134. The multivalent CD80 polypeptide of embodiment 133, wherein the affinity is increased more than 1.2-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 80-fold, 100-fold, 150-fold, 200-fold, 250-fold, 300-fold, 400-fold, or 450-fold compared to binding affinity of the unmodified CD80 for the ectodomain of the binding partner.

135. The multivalent CD80 polypeptide of any of embodiments 129-134, wherein the one or more amino acid modifications are amino acid substitutions.

136. The multivalent CD80 polypeptide of any of embodiments 129-135, wherein the one or more amino acid modifications comprise one or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

137. The multivalent CD80 polypeptide of any of embodiments 129-136, wherein the one or more amino acid modifications comprise two or more amino acid substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

138. The multivalent CD80 polypeptide of any of embodiments 129-137, wherein the one or more amino acid modifications comprises amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2.

139. The multivalent CD80 polypeptide of any of embodiments 129-138, wherein the one or more amino acid modifications comprise amino acid substitutions E35D/M47L/V68M, E35D/M47V/V68M or E35D/M47I/L70M.

140. The multivalent CD80 polypeptide of any of embodiments 129-139, wherein the one or more amino acid modifications comprise amino acid substitutions E35D/M47V/N48K/V68M/K89N, H18Y/A26E/E35D/M47L/V68M/A71G/D90G, E35D/D46E/M47V/V68M/D90G/K93E or E35D/D46V/M47L/V68M/L85Q/E88D.

141. The multivalent CD80 polypeptide of any of embodiments 129-140, wherein the unmodified CD80 is a human CD80.

142. The multivalent CD80 polypeptide of any of embodiments 129-141, wherein the extracellular domain or portion thereof of the unmodified CD80 comprises (i) the sequence of amino acids set forth in SEQ ID NO:2, (ii) a sequence of amino acids that has at least 95% sequence identity to SEQ ID NO:2; or (iii) is a portion of (i) or (ii) comprising an IgV domain or a specific binding fragment thereof.

143. The multivalent CD80 polypeptide of embodiment 142, wherein the extracellular domain or portion thereof of the unmodified CD80 is an extracellular domain portion that is or comprises the IgV domain or a specific binding fragment thereof.

144. The multivalent CD80 polypeptide of embodiment 143, wherein the extracellular domain portion of the unmodified CD80 comprises the IgV domain but does not comprise the IgC domain or a portion of the IgC domain.

145. The multivalent CD80 polypeptide of embodiment 143 or embodiment 144, wherein the extracellular domain portion of the unmodified CD80 is set forth as the sequence of amino acids 35-135 of SEQ ID NO:2 (SEQ ID NO:76) or 35-141 of SEQ ID NO:2 (SEQ ID NO:150).

146. The multivalent CD80 polypeptide of any of embodiments 129-145, wherein the vCD80 is an extracellular domain portion that does not comprise the IgC domain or a portion of the IgC domain.

147. The multivalent CD80 polypeptide of any of embodiments 129-146, wherein the vCD80 comprises the sequence of amino acids 35-135 of SEQ ID NO:2 (SEQ ID NO:76) or 35-141 of SEQ ID NO:2 (SEQ ID NO:150) in which is contained the one or more amino acid substitutions.

148. The multivalent CD80 polypeptide of any of embodiments 129-147, wherein the vCD80 has the sequence of amino acids 35-135 of SEQ ID NO:2 (SEQ ID NO:76) or 35-141 of SEQ ID NO:2 (SEQ ID NO:150) in which is contained the one or more amino acid substitutions.

149. The multivalent CD80 polypeptide of any of embodiments 129-148, wherein the vCD80 comprises 1, 2, 3, 4, 5, 6, 7, 8, 9, 10, 11, 12, 13, 14, 15, 16, 17, 18, 19 or 20 amino acid modifications, optionally wherein the amino acid modifications are amino acid substitutions.

150. The multivalent CD80 polypeptide of any of embodiments 129-149, wherein the vCD80 comprises no more than 1, 2, 3, 4, 5, 6, 7, 8, 9, 10, 11, 12 or 13 amino acid modifications, optionally wherein the amino acid modifications are amino acid substitutions.

151. The multivalent CD80 polypeptide of any of embodiments 129-150, wherein the vCD80 has at least or at least about 85%, 86%, 87%, 88%, 89%, 90%, 91%, 92%, 93%, 94%, 95%, 96%, 97%, 98%, 99% or more sequence identity to the sequence of amino acids 35-135 of SEQ ID NO:2 (SEQ ID NO:76) or 35-141 of SEQ ID NO:2 (SEQ ID NO:150).

152. The multivalent CD80 polypeptide of any of embodiments 129-151, wherein the multimerization domain is an Fc region.

153. The multivalent CD80 polypeptide of any of embodiments 129-152, wherein the Fc region is of an immunoglobulin G1 (IgG1) or an immunoglobulin G2 (IgG2) protein.

154. The multivalent CD80 polypeptide of embodiment 152 or embodiment 153, wherein the Fc region exhibits one or more effector functions.

155. The multivalent CD80 polypeptide of embodiment 154 or embodiment 153, wherein the Fc region is a variant Fc region comprising one or more amino acid substitutions in a wildtype Fc region, said variant Fc region exhibiting one or more effector function that is reduced compared to the wildtype Fc region, optionally wherein the wildtype human Fc is of human IgG1.

156. The multivalent CD80 polypeptide of any of embodiments 129-155, wherein each vCD80 is the same.

157. The multivalent CD80 polypeptide of any of embodiments 129-156, wherein the linker is a flexible linker.

158. The multivalent CD80 polypeptide of any of embodiments 129-157, wherein the linker is a peptide linker.

159. The multivalent CD80 polypeptide of embodiment 158, wherein the linker is GSGGGGS (SEQ ID NO:1522) or 3× GGGGS (SEQ ID NO: 1504).

160. A nucleic acid molecule encoding the multivalent CD80 polypeptide of any of embodiments 129-159.

161. A vector comprising the nucleic acid of embodiment 160.

162. The vector of embodiment 161 that is an expression vector.

163. A host cell comprising the nucleic acid of embodiment 160 or the vector of embodiment 161 or embodiment 162.

164. A method of producing a multivalent CD80 polypeptide of any of embodiments 129-159, comprising introducing the nucleic acid of embodiment 160 or the vector of embodiment 161 or embodiment 162 into a host cell under conditions to express the protein in the cell.

165. The method of embodiment 164, further comprising isolating or purifying the protein comprising the multivalent CD80 polypeptide.

166. A pharmaceutical composition comprising the multivalent CD80 polypeptide of any of embodiments 129-159.

167. The pharmaceutical composition of embodiment 166, comprising a pharmaceutically acceptable excipient.

168. The pharmaceutical composition of embodiment 166 or embodiment 167, wherein the pharmaceutical composition is sterile.

169. An article of manufacture comprising the pharmaceutical composition of any of embodiments 166-168 in a container, optionally wherein the container is a vial.

170. The article of manufacture of embodiment 169, wherein the container is sealed.

171. A method of modulating an immune response in a subject, comprising administering the pharmaceutical composition of any of embodiments 166-168 to a subject or the multivalent CD80 polypeptide of any of embodiments 129-170 to a subject.

172. The method of any of embodiment 171, wherein modeling the immune response treats a disease or condition in the subject.

173. The method of embodiment 172, wherein the disease or condition is a tumor or cancer.

174. A method of treating a cancer in a subject, comprising administering the pharmaceutical composition of any of embodiments 166-168 to a subject or the multivalent CD80 polypeptide of any of embodiments 129-171 to a subject.

175. A variant CD80 fusion protein comprising: (i) a variant extracellular domain comprising one or more amino acid substitutions at one or more positions in the sequence of amino acids set forth as amino acid residues 35-230 of a wildtype human CD80 extracellular domain corresponding to residues set forth in SEQ ID NO:1 and (ii) an Fc region that has effector activity, wherein the extracellular domain of the variant CD80 fusion protein specifically binds to the ectodomain of human CD28 and does not bind to the ectodomain of human PD-L1 or binds to the ectodomain of PD-L1 with a similar binding affinity as the extracellular domain of the wildtype human CD80 for the ectodomain of PD-L1.

176. The variant CD80 fusion protein of embodiment 175, wherein the extracellular domain of the variant CD80 fusion protein exhibits increased binding affinity to the ectodomain of human CTLA-4 compared to the binding affinity of the extracellular domain of wildtype CD80 for the ectodomain of human CTLA-4.

177. The variant CD80 fusion protein of embodiment 175 or embodiment 176, wherein the extracellular domain of the variant CD80 fusion protein exhibits increased binding affinity to the ectodomain of human CD28 compared to the binding affinity of the extracellular domain of wildtype CD80 for the ectodomain of human CD28.

178. The variant CD80 fusion protein of embodiment 176 or embodiment 177, wherein the affinity is increased about or greater than 1.2-fold, 1.5-fold, 2-fold, 3-fold, 4-fold, 5-fold, 6-fold, 7-fold, 8-fold, 9-fold, 10-fold or more.

179. The variant CD80 fusion protein of any of embodiments 175-178, wherein:

the variant CD80 fusion protein increases immunological activity as assessed in a mixed lymphocyte reaction, optionally wherein the increased immunological activity comprises increased production of IFN-gamma or interleukin 2 in the mixed lymphocyte reaction; and/or

the variant CD80 fusion protein increases immunological activity as assessed in a T cell reporter assay incubated with antigen presenting cells.

180. The variant CD80 fusion protein of any of embodiments 175-179, wherein the variant CD80 fusion protein increases CD28-mediated costimulation of T lymphocytes.

181. The variant CD80 fusion protein of embodiment 179 or embodiment 180, wherein the increase is by about or greater than 1.2-fold, 1.5-fold, 2-fold, 3-fold, 4-fold, 5-fold, 6-fold, 7-fold, 8-fold, 9-fold, 10-fold or more.

182. The variant CD80 fusion protein of any of embodiments 175-181, wherein the wildtype human CD80 extracellular domain has the sequence of amino acids set forth in SEQ ID NO:2 or a sequence that has at least 95%, 96%, 97%, 98%, 99% or more sequence identity to SEQ ID NO:2.

183. The variant CD80 fusion protein of any of embodiments 175-182, wherein the wildtype human CD80 extracellular domain has the sequence of amino acids set forth in SEQ ID NO:2.

184. The variant CD80 fusion protein of any of embodiments 175-183, wherein the one or more amino acid substitutions comprise one or more amino acid substitutions selected from L70Q, K89R, D90G, D90K, A91G, F92Y, K93R, I118V, T120S or T130A, with reference to numbering set forth in SEQ ID NO:2, or a conservative amino acid substitution thereof.

185. The variant CD80 fusion protein of any of embodiments 175-184, wherein the one or more amino acid substitutions comprise two or more amino acid substitutions selected from L70Q, K89R, D90G, D90K, A91G, F92Y, K93R, I118V, T120S or T130A, with reference to numbering set forth in SEQ ID NO:2, or a conservative amino acid substitution thereof.

186. The variant CD80 fusion protein of embodiment 184 or embodiment 185, wherein the one or more amino acid substitutions comprise amino acid modifications L70Q/K89R, L70Q/D90G, L70Q/D90K, L70Q/A91G, L70Q/F92Y, L70Q/K93R, L70Q/I118V, L70Q/T120S, L70Q/T130A, K89R/D90G, K89R/D90K, K89R/A91G, K89R/F92Y, K89R/K93R, K89R/I118V, K89R/T120S, K89R/T130A, D90G/A91G, D90G/F92Y, D90G/K93R, D90G/I118V, D90G/T120S, D90G/T130A, D90K/A91G, D90K/F92Y, D90K/K93R, D90K/I118V, D90K/T120S, D90K/T130A, F92Y/K93R, F92Y/I118V, F92Y/T120S, F92Y/T130A, K93R/I118V, K93R/T120S, K93R/T130A, I118V/T120S, I118V/T130A or T120S/T130A.

187. The variant CD80 fusion protein of any of embodiments 175-186, wherein the one or more amino acid substitutions comprise amino acid substitutions A91G/I118V/T120S/T130A.

188. The variant CD80 fusion protein of any of embodiments 175-186, wherein the one or more amino acid substitutions comprise amino acid substitutions S21P/L70Q/D90G/I118V/T120S/T130A.

189. The variant CD80 fusion protein of any of embodiments 175-186, wherein the one or more amino acid substitutions comprise amino acid substitutions E88D/K89R/D90K/A91G/F92Y/K93R.

190. The variant CD80 fusion protein of any of embodiments 175-183, wherein the one or more amino acid substitutions comprise one or more amino acid substitutions selected from substitutions selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

191. The variant CD80 fusion protein of embodiment 190, wherein the one or more amino acid substitutions comprises amino acid substitutions H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2.

192. The variant CD80 fusion protein of any of embodiments 175-183, 190 and 191, wherein the one or more amino acid modifications comprise amino acid substitutions E35D/M47L/V68M, E35D/M47V/V68M or E35D/M47I/L70M.

193. The variant CD80 fusion protein of any of embodiments 175-183, and 190-192, wherein the one or more amino acid modifications comprise amino acid substitutions E35D/M47V/N48K/V68M/K89N, H18Y/A26E/E35D/M47L/V68M/A71G/D90G, E35D/D46E/M47V/V68M/D90G/K93E or E35D/D46V/M47L/V68M/L85Q/E88D.

194. The variant CD80 fusion protein of any of embodiments 175-193, wherein the variant CD80 extracellular domain has 1, 2, 3, 4, 5, 6, 7, 8, 9, 10, 11, 12, 13, 14, 15, 16, 17, 18, 19 or 20 amino acid substitutions.

195. The variant CD80 fusion protein of any of embodiments 175-194, wherein the variant CD80 extracellular domain comprises no more than 1, 2, 3, 4, 5, 6, 7, 8, 9, 10, 11, 12 or 13 amino acid substitutions.

196. The variant CD80 fusion protein of any of embodiments 175-195, wherein the variant CD80 extracellular domain has at least or at least about 85%, 86%, 87%, 88%, 89%, 90%, 91%, 92%, 93%, 94%, 95%, 96%, 97%, 98%, 99% or more sequence identity to the sequence of amino acids set forth in SEQ ID NO:2.

197. The variant CD80 fusion protein of any of embodiments 175-196, wherein the Fc region is of an immunoglobulin G1 (IgG1).

198. The variant CD80 fusion protein of any of embodiments 175-197, wherein the Fc region comprises the amino acid substitution C220S, wherein the residues are numbered according to the EU index of Kabat.

199. The variant CD80 fusion protein of any of embodiments 175-198, wherein the Fc region comprises K447del, wherein the residue is numbered according to the EU index of Kabat.

200. The variant CD80 fusion protein of any of embodiments 175-199, wherein the Fc region as the sequence of amino acids set forth in SEQ ID NO: 1502, 1510, 1517 or 1527.

201. The variant CD80 fusion protein of any of embodiments 175-200, wherein the one or more effector function is selected from among antibody dependent cellular cytotoxicity (ADCC), complement dependent cytotoxicity, programmed cell death and cellular phagocytosis.

202. The variant CD80 fusion protein of any of embodiments 175-201 that is a dimer.

203. A nucleic acid molecule encoding the variant CD80 fusion protein of any of embodiments 175-202.

204. A vector comprising the nucleic acid of embodiment 203.

205. The vector of embodiment 204 that is an expression vector.

206. A host cell comprising the nucleic acid of embodiment 203 or the vector of embodiment 204 or embodiment 205.

207. A method of producing a variant CD80 fusion protein of any of embodiments 175-202, comprising introducing the nucleic acid of embodiment 203 or the vector of embodiment 204 or embodiment 205 into a host cell under conditions to express the protein in the cell.

208. The method of embodiment 207, further comprising isolating or purifying the protein comprising the variant CD80 fusion protein.

209. A pharmaceutical composition comprising the variant CD80 fusion protein of any of embodiments 175-202.

210. The pharmaceutical composition of embodiment 209, comprising a pharmaceutically acceptable excipient.

211. The pharmaceutical composition of embodiment 209 or embodiment 210, wherein the pharmaceutical composition is sterile.

212. An article of manufacture comprising the pharmaceutical composition of any of embodiments 209-211 in a container, optionally wherein the container is a vial.

213. The article of manufacture of embodiment 212, wherein the container is sealed.

214. A method of modulating an immune response in a subject, comprising administering the pharmaceutical composition of any of embodiments 209-211 to a subject or the variant CD80 fusion protein of any of embodiments 175-202 to a subject.

215. The method of any of embodiment 214, wherein modulating the immune response treats a disease or condition in the subject.

216. The method of embodiment 215, wherein the disease or condition is a tumor or cancer.

217. A method of treating a cancer in a subject, comprising administering the pharmaceutical composition of any of embodiments 209-211 to a subject or the variant CD80 fusion protein of any of embodiments 175-202 to a subject.

### VII. EXAMPLES

The following examples are included for illustrative purposes only and are not intended to limit the scope of the invention.

### Example 1

### Generation of Mutant DNA Constructs of IgSF Domains

Example 1 describes the generation of mutant DNA constructs of human CD80 IgSF domains for translation and expression on the surface of yeast as yeast display libraries.

A. Degenerate Libraries

Constructs were generated based on a wildtype human CD80 sequence set forth in SEQ ID NO:150, containing the immunoglobulin-like V-type (IgV) domain as follows:

(SEQ ID NO: 150)
VIHVTKEVKEVATLSCGHNVSVEELAQTRIYWQKE
KKMVLTMMSGDMNIWPEYKNRTIFDITNNLSIVIL
ALRPSDEGTYECVVLKYEKDAFKREHLAEVTLSV
KAD

For libraries that target specific residues for complete or partial randomization with degenerate codons, degenerate codons, such as specific mixed base sets to code for various amino acid substitutions, were generated using an algorithm at the URL: rosettadesign.med.unc.edu/SwiftLib/. In general, positions to mutate were chosen from crystal structure information for CD80 bound to CTLA-4 at the URL: rcsb.org/pdb/explore/explore.do?structureId=1I8L, and a targeted library was designed based on the CD80::CTLA-4 interface for selection of improved binders to CTLA-4. For example, the structural information was used to identify contact or non-contact interface residues for mutagenesis with degenerate codons. This analysis was performed using a structure viewer available at the URL: spdbv.vital-it.ch.

The next step in library design was the alignment of human, mouse, rat, and monkey CD80 sequences to identify which of the residues chosen for mutagenesis were conserved residues. Based on this analysis, conserved target residues were mutated with degenerate codons that only specified conservative amino acid changes plus the wild-type residue. Residues that were not conserved were mutated more aggressively, but also included the wild-type residue. Degenerate codons that also encoded the wild-type residue were deployed to avoid excessive mutagenesis of target protein. For the same reason, only up to 20 positions were targeted for mutagenesis for each library. Mutational analysis was focused on contact and non-contact interfacial residues that were within 6 Å of the binding surface with their side chains directed toward the ligand/counter structure.

To generate DNA encoding the targeted library, overlapping oligos of up to 80 nucleotides in length and containing degenerate codons at the residue positions targeted for mutagenesis, were ordered from Integrated DNA Technologies (Coralville, USA). The oligonucleotides were dissolved in sterile water, mixed in equimolar ratios, heated to 95° C. for five minutes and slowly cooled to room temperature for annealing. IgV domain-specific oligonucleotide primers that anneal to the start and end of the IgV domain gene sequence were then used to generate PCR product. IgV domain-specific oligonucleotides which overlap by 40 bp with pBYDS03 cloning vector (Life Technologies, USA), beyond and including the BamHI and KpnI cloning sites, were then used to amplify 100 ng of PCR product from the prior step to generate a total of at least 12 μg of DNA for every electroporation. Both polymerase chain reactions (PCRs) used OneTaq 2× PCR master mix (New England Biolabs, USA). The products from the second PCR were purified using a PCR purification kit (Qiagen, Germany) and resuspended in sterile deionized water. Alternatively, Ultramers® (Integrated DNA Technologies) of up to 200 bp in length were used in conjunction with megaprimer PCR (URL: http://www.ncbi.nlm.nih.gov/pmc/articles/PMC146891/pdf/253371.pdf) to generate larger stretches of degenerate codons that could not be as easily incorporated using multiple small overlapping primers. Following the generation of full length product using megaprimer PCR, the mutant IgV domain library was PCR amplified again using DNA primers containing 40 bp overlap region with pBYDS03 cloning variant for homologous recombination into yeast.

To prepare for library insertion, pBYDS03 vector was digested with BamHI and KpnI restriction enzymes (New England Biolabs, USA) and the large vector fragment was gel-purified and dissolved in sterile, deionized water. Electroporation-ready DNA for the next step was generated by mixing 12 μg of library DNA insert with 4 μg of linearized vector in a total volume of 50 μL deionized and sterile water. An alternative method to generate targeted libraries, is to carry out site-directed mutagenesis (Multisite kit, Agilent, USA) of the target IgV domain with oligonucleotides containing degenerate codons. This approach is used to generate sublibraries that only target a few specific stretches of DNA for mutagenesis. In these cases, sublibraries are mixed before proceeding to the selection steps. In general, library sizes were in the range of 10E7 to 10E8 clones, except that sublibraries were only in the range of 10E4 to 10E5.

B. Random Libraries

Random libraries were also constructed to identify variants of the IgV domain of CD80 set forth in SEQ ID NO:150 (containing the IgV domain). DNA encoding the wild-type CD80 IgV domain was cloned between the BamHI and KpnI sites of yeast display vector pBYDS03 and then released using the same restriction enzymes. The released DNA was then mutagenized with the Genemorph II Kit (Agilent Genomics, USA) to generate an average of three to five amino acid changes per library variant. Mutagenized DNA was then amplified by the two-step PCR and further processed as described above for targeted libraries.

After completing several rounds of selection using beads and iterative FACS, a pool of clones were further mutated via error prone PCR. Thus, a second generation mutant library was created following the steps outlined as above though using selection output DNA as template rather than wildtype IgV plasmid sequence as template.

### Example 2

### Introduction of DNA Libraries into Yeast

To introduce degenerate and random CD80 library DNA into yeast, electroporation-competent cells of yeast strain BJ5464 (ATCC.org; ATCC number 208288) were prepared and electroporated on a Gene Pulser II (Biorad, USA) with the electroporation-ready DNA from the steps above essentially as described (Colby, D. W. et al. 2004 Methods Enzymology 388, 348-358). The only exception was that transformed cells were grown in non-inducing minimal selective SCD-Leu medium to accommodate the LEU2 selective marker carried by modified plasmid pBYDS03. One liter of SCD-Leu media consists of 14.7 grams of sodium citrate, 4.29 grams of citric acid monohydrate, 20 grams of dextrose, 6.7 grams of yeast nitrogen base, and 1.6 grams yeast synthetic drop-out media supplement without leucine. The Medium was filter sterilized before use, using a 0.22 μm vacuum filter device.

Library size was determined by plating dilutions of freshly recovered cells on SCD-Leu agar plates and then extrapolating library size from the number of single colonies from plating that generated at least 50 colonies per plate. The remainder of the electroporated culture was grown to saturation and cells from this culture were subcultured 1/100 into the same medium once more and grown to saturation to minimize the fraction of untransformed cells and to allow for segregation of plasmid from cells that may contain two or more library variants. To maintain library diversity, this subculturing step was carried out using an inoculum that contained at least 10× more cells than the calculated library size. Cells from the second saturated culture were resuspended in fresh medium containing sterile 25% (weight/volume) glycerol to a density of 10E10/mL and frozen and stored at −80° C. (frozen library stock).

### Example 3

### Yeast Selection

Example 3 describes the selection of yeast cells expressing affinity-modified variants of CD80. It has been well-established that CTLA-4 binding to CD80 antagonizes CD28 binding to CD80 (Schwartz J. C. et al. Nature 410, 604-08, 2001). To identify CD80 mutants that selectively bind CTLA-4 over CD28, cells from the CD80 mutant libraries were subjected to iterative rounds of positive and negative FACS sorting and mutagenesis.

A number of cells equal to at least 10 times the estimated library size were thawed from individual library stocks, suspended to 1.0×10E6 cells/mL in non-inducing SCD-Leu medium, and grown overnight. The next day, a number of cells equal to 10 times the library size were centrifuged at 2000 RPM for two minutes and resuspended to 0.5×10E6 cells/mL in inducing SCDG-Leu media. One liter of SCDG-Leu induction media consists of 5.4 grams Na₂HPO₄, 8.56 grams NaH₂PO₄.H₂O, 20 grams galactose, 2.0 grams dextrose, 6.7 grams yeast nitrogen base, and 1.6 grams yeast synthetic drop out media supplement without leucine dissolved in water and sterilized through a 0.22 μm membrane filter device. The culture was grown in induction medium for 1 day at room temperature to induce expression of library proteins on the yeast cell surface.

Cells were sorted twice using Protein A magnetic beads (New England Biolabs, USA) loaded with cognate ligand to reduce non-binders and enrich for all CD80 variants with the ability to bind their exogenous recombinant counter-structure proteins. This was then followed by multiple rounds of fluorescence activated cell sorting (FACS) using exogenous counter-structure protein staining to enrich the fraction of yeast cells that displays improved binding to CTLA-4-Fc (R&D Systems, USA). These positive selections were alternated with negative FACS selections to remove CD80 clones that bound to CD28-Fc. Magnetic bead enrichment and selections by flow cytometry were carried out essentially as described in Miller K. D., et al., Current Protocols in Cytometry 4.7.1-4.7.30, July 2008.

With CD80 libraries, target ligand proteins were employed as follows: internally produced human rCTLA-4-Fc, human rCD28-Fc, and human rPD-L1 (R&D Systems, Minneapolis, USA). Magnetic Protein A beads were obtained from New England Biolabs, USA. For two-color, flow cytometric sorting, a Bio-Rad S3e sorter was used. CD80 display levels were monitored with an anti-hemagglutinin (HA) antibody labeled with Alexafluor 488 (Life Technologies, USA). Ligand binding of Fc fusion proteins, rCTLA-4Fc, rPD-L1 or rCD28Fc, were detected with PE conjugated human Ig specific goat Fab (Jackson ImmunoResearch, USA). Doublet yeast were gated out using forward scatter (FSC)/side scatter (SSC) parameters, and sort gates were based upon higher ligand binding detected in FL2 that possessed more limited tag expression binding in FL1.

Yeast outputs from the flow cytometric sorts were assayed for higher specific binding affinity. Sort output yeast were expanded and re-induced to express the particular IgSF affinity modified domain variants they encode. This population then can be compared to the parental, wild-type yeast strain, or any other selected outputs, such as the bead output yeast population, by flow cytometry.

For CD80, the second FACS outputs (F2) were compared to parental CD80 yeast for binding rCTLA-4Fc, rPD-L1, or rCD28Fc by double staining each population with anti-HA (hemagglutinin) tag expression and the anti-human Fc secondary to detect ligand binding.

Selected variant CD80 IgV domains were further formatted as fusion proteins and tested for binding and functional activity as described below.

### Example 4

### Reformatting Selection Outputs as Fc-Fusions and in Various Immunomodulatory Protein Types

Example 4 describes reformatting of selection outputs identified in Example 3 as immunomodulatory proteins containing an affinity modified (variant) immunoglobulin-like V-type (IgV) domain of CD80 fused to an Fc molecule (variant IgV domain-Fc fusion molecules).

Output cell pools from final flow cytometric CD80 sorts were grown to terminal density in SCD-Leu medium. Plasmid DNA from each output was isolated using a yeast plasmid DNA isolation kit (Zymoresearch, USA). For Fc fusions, PCR primers with added restriction sites suitable for cloning into the Fc fusion vector of choice were used to batch-amplify from the plasmid DNA preps the coding DNA for the mutant target IgV domains After restriction digestion, the PCR products were ligated into Fc fusion vector followed by heat shock transformation into E. coli strain XL1 Blue (Agilent, USA) or NEB5alpha (New England Biolabs) as directed by supplier. Alternatively, the outputs were PCR amplified with primers containing 40 bp overlap regions on either end with Fc fusion vector to carry out in vitro recombination using Gibson Assembly Mastermix (New England Biolabs), which was subsequently used in heat shock transformation into E. coli strain NEB5alpha. Exemplary of an Fc fusion vector is pFUSE-hIgG1-Fc2 (InvivoGen, USA).

Dilutions of transformation reactions were plated on LB-agar containing 100 μg/mL carbenicillin (Teknova, USA) to isolate single colonies for selection. Up to 96 colonies from each transformation were then grown in 96 well plates to saturation overnight at 37° C. in LB-carbenicillin broth (Teknova cat #L8112) and a small aliquot from each well was submitted for DNA sequencing of the IgV domain insert in order to identify the mutation(s) in all clones. Sample preparation for DNA sequencing was carried out using protocols provided by the service provider (Genewiz; South Plainfield, N.J.). After removal of sample for DNA sequencing, glycerol was then added to the remaining cultures for a final glycerol content of 25% and plates were stored at −20° C. for future use as master plates (see below). Alternatively, samples for DNA sequencing were generated by replica plating from grown liquid cultures onto solid agar plates using a disposable 96 well replicator (VWR, USA). These plates were incubated overnight to generate growth patches and the plates were submitted to Genewiz as specified by Genewiz.

After identification of clones of interest from analysis of Genewiz-generated DNA sequencing data, clones of interest were recovered from master plates and individually grown to density in liquid LB-broth containing 100 μg/mL carbenicillin (Teknova, USA) and cultures were then used for preparation of plasmid DNA of each clone using a standard kit such as the PureYield Plasmid Miniprep System (Promega) or the MidiPlus kit (Qiagen). Identification of clones of interest from Genewiz sequencing data generally involved the following steps. First, DNA sequence data files were downloaded from the Genewiz website. All sequences were then manually curated so that they start at the beginning of the IgV domain coding region. The curated sequences were then batch-translated using a suitable program available at the URL: www.ebi.ac.uk/Tools/st/emboss_transeq/. The translated sequences were then aligned using a suitable program available at the URL: multalin.toulouse.inra.fr/multalin/multalin.html. Alternatively, Genewiz sequenced were processed to generate alignments using Ugene software (http://ugene.net).

Clones of interest were then identified from alignments using the following criteria: 1) identical clone occurs at least two times in the alignment and 2) a mutation occurs at least two times in the alignment and preferably in distinct clones. Clones that meet at least one of these criteria were assumed to be clones that have been enriched by the sorting process due to improved binding.

To generate recombinant immunomodulatory proteins that are Fc fusion proteins containing an IgV domain of CD80 with at least one affinity-modified domain (e.g. variant CD80 IgV-Fc), the DNA encoding the variant was generated to encode a protein as follows: variant (mutant) CD80 IgV domain followed by a linker of three alanines (AAA) followed by an inert Fc lacking effector function. In some cases the inert Fc was an Fc containing the mutations C220S, R292C, N297G and V302C by EU numbering (corresponding to C5S, R77C, N82G and V87C with reference to wild-type human IgG1 Fc set forth in SEQ ID NO: 1502), such as set forth in set forth in SEQ ID NO: 1519. In some cases, the inert Fc was an Fc containing the mutations C220S, L234A, L235E and G237A by EU numbering, such as set forth in SEQ ID NO: 1518 or 1520. Alternatively, CD80 IgV domains were fused in a similar manner but with a linker containing the amino acids (GSGGGGS; SEQ ID NO: 1522) followed by an inert Fc lacking effector function, set forth in SEQ ID NO: 1520, or allotypes thereof. In some cases, CD80 IgV domains were fused in a similar manner but with a human IgG1 Fc capable of effector activity (effector). Since the construct does not include an antibody, light chains that can form a covalent bond with a cysteine, such an exemplary human IgG1 Fc (set forth in SEQ ID NO: 1517) contained a replacement of the cysteine residue to a serine residue at position 220 (C220S) by EU numbering (corresponding to position 5 (C5S) with reference to the wild-type or unmodified Fc set forth in SEQ ID NO: 1502).

### Example 5

### Expression and Purification of Fc-Fusions

Example 5 describes the high throughput expression and purification of Fc-fusion proteins containing variant IgV CD80 as described in the above Examples.

Recombinant variant Fc fusion proteins were produced from suspension-adapted human embryonic kidney (HEK) 293 cells using the Expi293 expression system (Invitrogen, USA). 4 μg of each plasmid DNA from the previous step was added to 200 μL Opti-MEM (Invitrogen, USA) at the same time as 10.8 μL ExpiFectamine was separately added to another 200 μL Opti-MEM. After 5 minutes, the 200 μL of plasmid DNA was mixed with the 200 μL of ExpiFectamine and was further incubated for an additional 20 minutes before adding this mixture to cells. Ten million Expi293 cells were dispensed into separate wells of a sterile 10 mL, conical bottom, deep 24-well growth plate (Thomson Instrument Company, USA) in a volume of 4 mL Expi293 media (Invitrogen, USA). Plates were shaken for 5 days at 120 RPM in a mammalian cell culture incubator set to 95% humidity and 8% CO₂. Following a 5-day incubation, cells were pelleted and culture supernatants were retained.

Proteins were purified from supernatants using a high throughput 96-well Filter Plate (Thomson Catalog number 931919), each well loaded with 60 μL of Mab SelectSure settled bead (GE Healthcare cat. no. 17543801). Protein was eluted with four consecutive 200 μl fractions of 50 mM Acetate pH 3.3. Each fraction's pH was adjusted to above pH 5.0 with 4 μL 2 M Tris pH 8.0. Fractions were pooled and quantitated using 280 nm absorbance measured by Nanodrop instrument (Thermo Fisher Scientific, USA), and protein purity was assessed by loading 5 μg of non-reduced protein on Mini-Protean TGX Stain-Free gels. Proteins were then visualized on a Bio Rad Chemi Doc XRS gel imager.

### Example 6

### Assessment of Binding of Affinity-Matured IgSF Domain-Containing Molecules

This Example describes Fc-fusion binding studies of purified proteins from the above Examples to cell-expressed CTLA-4, PD-L1, and CD28 counter structures to assess the specificity and affinity of CD80 domain variant immunomodulatory proteins. Full-length mammalian surface expression constructs for each of human CTLA-4, PD-L1, and CD28, were designed in pcDNA3.1 expression vector (Life Technologies) and sourced from Genscript, USA. Binding studies were carried out on transfected HEK293 cells generated to express the full-length mammalian surface ligands using the using the Expi293F transient transfection system (Life Technologies, USA). As a control, binding to mock (non-transfected) cells also was assessed. The number of cells needed for the experiment was determined, and the appropriate 30 mL scale of transfection was performed using the manufacturer's suggested protocol. For each CTLA-4, PD-L1, CD-28 or mock 30 mL transfection, 75 million Expi293F cells were incubated with 30 μg expression construct DNA and 1.5 mL diluted ExpiFectamine 293 reagent for 48 hours, at which point cells were harvested for staining.

For staining and analysis by flow cytometry, 100,000 cells of appropriate transient transfection or negative control (mock) were plated in 96-well round bottom plates. Cells were spun down and resuspended in staining buffer (PBS (phosphate buffered saline), 1% BSA (bovine serum albumin), and 0.1% sodium azide) for 20 minutes to block non-specific binding. Afterwards, cells were centrifuged and resuspended in staining buffer containing 200 nM to 91 pM of each candidate variant CD80 Fc, depending on the experiment of each candidate variant CD80 Fc protein in 50 μl. As controls, the binding activities of wild-type CD80-ECD-Fc (R&D Systems), wild-type CD80-ECD-Fc (inert), wild-type IgV-Fc (inert) and/or human IgG (Sigma) were also assessed. Primary staining was performed on ice for 45 minutes, before washing cells in staining buffer twice. PE-conjugated anti-human Fc (Jackson ImmunoResearch, USA) was diluted 1:150 in 50 μL staining buffer and added to cells and incubated another 30 minutes on ice. Secondary antibody was washed out twice, cells were fixed in 4% formaldehyde/PBS, and samples were analyzed on Intellicyt flow cytometer (Intellicyt Corp, USA). Mean Fluorescence Intensity (MFI) was calculated for each transfectant and mock transfected HEK293 with FlowJo Version 10 software (FlowJo LLC, USA).

Results for two binding studies for exemplary CD80 variants are shown in Tables E1 and E2. In the Tables. The exemplary amino acid substitutions are designated by amino acid position number corresponding to numbering of the respective reference unmodified ECD sequence. For example, the reference unmodified ECD sequence is the unmodified CD80 ECD sequence set forth in SEQ ID NO: 2. The amino acid position is indicated in the middle, with the corresponding unmodified (e.g., wild-type) amino acid listed before the number and the identified variant amino acid substitution listed after the number. The second column sets forth the SEQ ID NO identifier for the variant IgV for each variant IgV-Fc fusion molecule.

Also shown is the binding activity as measured by the Mean Fluorescence Intensity (MFI) value for the binding of each variant CD80 Fc-fusion molecule to cells engineered to express the indicated cognate counter structure ligand (i.e., CTLA-4, PD-L1, or CD28) and the ratio of the MFI of the variant CD80 IgV-Fc, compared to the binding of the corresponding unmodified CD80 IgV-Fc fusion molecule not containing the amino acid substitution(s), to the same cell-expressed counter structure ligand. The ratio of the binding of the variant CD80IgV-Fc to the CTLA-4 counter structure ligand compared to the binding of the variant CD80IgV-Fc to the CD28 counter structure ligand also is shown in the last column of the Tables.

As shown in Tables E1 and E2, the selections resulted in the identification of a number of CD80 IgV domain variants that were affinity-modified to exhibit increased binding for CTLA-4 and/or PD-L1 counter structure ligand(s). In addition, the results indicate that a number of variants were selected that exhibit reduced binding to CD28, including several CD80 IgV domain variants that exhibit increased binding to the CTLA-4 counter structure ligand compared to the CD28 counter structure ligand (Ratio of CTLA-4:CD28).

TABLE E1
Variant CD80 Binding to HEK293 Cells Transfected with CTLA4, CD28 or PD-L1

| | | CTLA4 | | CD28 | | PD-L1 | | Ratio |
|---|---|---|---|---|---|---|---|---|
| | SEQ | MFI at | Fold | MFI at | Fold | MFI at | Fold | of |
| | ID NO | 66.6 | change | 66.6 | change | 22.2 | change | CTLA |
| CD80 mutation(s) | (IgV) | nM | to WT | nM | to WT | nM | to WT | 4:CD28 |
| L70P | 151 | Not tested | | | | | | |
| I30F/L70P | 152 | Not tested | | | | | | |
| Q27H/T41S/A71D | 153 | 368176 | 2.3 | 25051 | 1.01 | 24181 | N/A | 14.7 |
| I30T/L70R | 154 | 2234 | 0.0 | 2596 | 0.10 | 5163 | N/A | 0.9 |
| T13R/C16R/L70Q/A71D | 155 | 197357 | 1.2 | 16082 | 0.65 | 9516 | N/A | 12.3 |
| T57I | 156 | 393810 | 2.4 | 23569 | 0.95 | 3375 | N/A | 16.7 |
| M43I/C82R | 157 | 3638 | 0.0 | 3078 | 0.12 | 7405 | N/A | 1.2 |
| V22L/M38V/M47I/A71D/ | 158 | 175235 | 1.1 | 3027 | 0.12 | 6144 | N/A | 57.9 |
| L85M | | | | | | | | |
| I30V/T57I/L70P/A71D/ | 159 | 116085 | 0.7 | 10129 | 0.41 | 5886 | N/A | 11.5 |
| A91T | | | | | | | | |
| V22I/L70M/A71D | 160 | 163825 | 1.0 | 22843 | 0.92 | 33404 | N/A | 7.2 |
| N55D/L70P/E77G | 161 | Not tested | | | | | | |
| T57A/I69T | 162 | Not tested | | | | | | |
| N55D/K86M | 163 | 3539 | 0.0 | 3119 | 0.13 | 5091 | N/A | 1.1 |
| L72P/T57I | 164 | 50176 | 0.3 | 3397 | 0.14 | 6023 | N/A | 14.8 |
| L70P/F92S | 165 | 4035 | 0.0 | 2948 | 0.12 | 6173 | N/A | 1.4 |
| T79P | 166 | 2005 | 0.0 | 2665 | 0.11 | 4412 | N/A | 0.8 |
| E35D/M47I/L65P/D90N | 167 | 4411 | 0.0 | 2526 | 0.10 | 4034 | N/A | 1.7 |
| L25S/E35D/M47I/D90N | 168 | 61265 | 0.4 | 4845 | 0.20 | 20902 | N/A | 12.6 |
| A71D | 170 | 220090 | 1.4 | 16785 | 0.68 | 29642 | N/A | 13.1 |
| E81K/A91S | 172 | 98467 | 0.6 | 3309 | 0.13 | 44557 | N/A | 29.8 |
| A12V/M47V/L70M | 173 | 81616 | 0.5 | 7400 | 0.30 | 31077 | N/A | 11.0 |
| K34E/T41A/L72V | 174 | 88982 | 0.6 | 3755 | 0.15 | 35293 | N/A | 23.7 |
| T41S/A71D/V84A | 175 | 103010 | 0.6 | 5573 | 0.22 | 83541 | N/A | 18.5 |
| E35D/A71D | 176 | 106069 | 0.7 | 18206 | 0.73 | 40151 | N/A | 5.8 |
| E35D/M47I | 177 | 353590 | 2.2 | 14350 | 0.58 | 149916 | N/A | 24.6 |
| K36R/G78A | 178 | 11937 | 0.1 | 2611 | 0.11 | 5715 | N/A | 4.6 |
| Q33E/T41A | 179 | 8292 | 0.1 | 2442 | 0.10 | 3958 | N/A | 3.4 |
| M47V/N48H | 180 | 207012 | 1.3 | 14623 | 0.59 | 145529 | N/A | 14.2 |
| M47L/V68A | 181 | 74238 | 0.5 | 13259 | 0.53 | 11223 | N/A | 5.6 |
| S44P/A71D | 182 | 8839 | 0.1 | 2744 | 0.11 | 6309 | N/A | 3.2 |
| Q27H/M43I/A71D/R73S | 183 | 136251 | 0.8 | 12391 | 0.50 | 8242 | N/A | 11.0 |
| E35D/I57I/L70Q/A71D | 185 | 121901 | 0.8 | 21284 | 0.86 | 2419 | N/A | 5.7 |
| M47I/E88D | 186 | 105192 | 0.7 | 7337 | 0.30 | 97695 | N/A | 14.3 |
| M42I/I61V/A71D | 187 | 54478 | 0.3 | 6074 | 0.24 | 4226 | N/A | 9.0 |
| P51A/A71D | 188 | 67256 | 0.4 | 4262 | 0.17 | 5532 | N/A | 15.8 |
| H18Y/M47I/T57I/A71G | 189 | 136455 | 0.8 | 20081 | 0.81 | 13749 | N/A | 6.8 |
| V20I/M47V/I57I/V84I | 190 | 183516 | 1.1 | 26922 | 1.08 | 3583 | N/A | 6.8 |
| WT CD80 ECD-Fc | 2 | 161423 | 1.0 | 24836 | 1.00 | Not | N/A | 6.5 |
| | | | | | | tested | | |
| Fc only | | 5962 | | 2592 | | 4740 | | |

TABLE E2
Variant CD80 Binding to HEK293 Cells Transfected with CTLA4, CD28 or PD-L1

| | | | | CD28 | | | | |
|---|---|---|---|---|---|---|---|---|
| | | CTLA4 | | MFI | | PD-L1 | | |
| | SEQ | MFI at | Fold | at | Fold | MFI at | Fold | Ratio |
| | ID NO | 66.6 | change | 66.6 | change | 22.2 | change | of |
| CD80 mutation(s) | (IgV) | nM | to WT | nM | to WT | nM | to WT | CTLA4:CD28 |
| V20I/M47V/A71D | 191 | 149937 | 7.23 | 15090 | 9.33 | 9710 | 5.48 | 9.9 |
| A71D/L72V/E95K | 192 | 140306 | 6.77 | 6314 | 3.90 | 8417 | 4.75 | 22.2 |
| V22L/E35G/A71D/L72P | 193 | 152588 | 7.36 | 8150 | 5.04 | 1403 | 0.79 | 18.7 |
| E35D/A71D | 194 | 150330 | 7.25 | 14982 | 9.26 | 13781 | 7.77 | 10.0 |
| E35D/I67L/A71D | 195 | 146087 | 7.04 | 11175 | 6.91 | 9354 | 5.28 | 13.1 |
| T13R/M42V/M47I/A71D | 197 | 108900 | 5.25 | 16713 | 10.33 | 1869 | 1.05 | 6.5 |
| E35D | 198 | 116494 | 5.62 | 3453 | 2.13 | 25492 | 14.38 | 33.7 |
| E35D/M47I/L70M | 199 | 116531 | 5.62 | 14395 | 8.90 | 49131 | 27.71 | 8.1 |
| E35D/A71D/L72V | 200 | 134252 | 6.47 | 11634 | 7.19 | 13125 | 7.40 | 11.5 |
| E35D/M43L/L70M | 201 | 102499 | 4.94 | 3112 | 1.92 | 40632 | 22.92 | 32.9 |
| A26P/E35D/M43I/L85Q/ | 202 | 83139 | 4.01 | 5406 | 3.34 | 9506 | 5.36 | 15.4 |
| E88D | | | | | | | | |
| E35D/D46V/L85Q | 203 | 85989 | 4.15 | 7510 | 4.64 | 38133 | 21.51 | 11.4 |
| Q27L/E35D/M47I/T57I/ | | | | | | | | |
| L70Q/E88D | 204 | 59793 | 2.88 | 14011 | 8.66 | 1050 | 0.59 | 4.3 |
| Q27H/E35G/A71D/L72P/ | 196 | 85117 | 4.10 | 10317 | 6.38 | 1452 | 0.82 | 8.3 |
| T791 | | | | | | | | |
| M47V/I69F/A71D/V831 | 205 | 76944 | 3.71 | 15906 | 9.83 | 3399 | 1.92 | 4.8 |
| E35D/T57A/A71D/L85Q | 206 | 85724 | 4.13 | 3383 | 2.09 | 1764 | 0.99 | 25.3 |
| H18Y/A26T/E35D/A71D/ | 207 | 70878 | 3.42 | 6487 | 4.01 | 8026 | 4.53 | 10.9 |
| L85Q | | | | | | | | |
| E35D/M47L | 208 | 82410 | 3.97 | 11508 | 7.11 | 58645 | 33.08 | 7.2 |
| E23D/M42V/M43I/I58V/ | 209 | 37331 | 1.80 | 10910 | 6.74 | 2251 | 1.27 | 3.4 |
| L70R | | | | | | | | |
| V68M/L70M/A71D/E95K | 210 | 56479 | 2.72 | 10541 | 6.51 | 38182 | 21.53 | 5.4 |
| N55I/T57I/I69F | 211 | 2855 | 0.14 | 1901 | 1.17 | 14759 | 8.32 | 1.5 |
| E35D/M43I/A71D | 212 | 63789 | 3.08 | 6369 | 3.94 | 27290 | 15.39 | 10.0 |
| T41S/T57I/L70R | 213 | 59844 | 2.89 | 4902 | 3.03 | 19527 | 11.01 | 12.2 |
| H18Y/A71D/L72P/E88V | 214 | 68391 | 3.30 | 8862 | 5.48 | 1085 | 0.61 | 7.7 |
| V20I/A71D | 215 | 60323 | 2.91 | 10500 | 6.49 | 3551 | 2.00 | 5.7 |
| E23G/A26S/E35D/T62N/ | 216 | 59025 | 2.85 | 5484 | 3.39 | 10662 | 6.01 | 10.8 |
| A71D/L72V/L85M | | | | | | | | |
| A12T/E24D/E35D/D46V/ | 217 | 63738 | 3.07 | 7411 | 4.58 | 1221 | 0.69 | 8.6 |
| I61V/L72P/E95V | | | | | | | | |
| V22L/E35D/M43L/A71G/ | 218 | 2970 | 0.14 | 1498 | 0.93 | 1851 | 1.04 | 2.0 |
| D76H | | | | | | | | |
| E35G/K54E/A71D/L72P | 219 | 71899 | 3.47 | 3697 | 2.29 | 1575 | 0.89 | 19.4 |
| L70Q/A71D | 220 | 45012 | 2.17 | 18615 | 11.50 | 1692 | 0.95 | 2.4 |
| A26E/E35D/M47L/L85Q | 221 | 40325 | 1.94 | 2266 | 1.40 | 55548 | 31.33 | 17.8 |
| D46E/A71D | 222 | 69674 | 3.36 | 16770 | 10.36 | 22777 | 12.85 | 4.2 |
| Y31H/E35D/T41S/V68L/ | | | | | | | | |
| K93R/R94W | 223 | 3379 | 0.16 | 2446 | 1.51 | 18863 | 10.64 | 1.4 |
| WT CD80 IgV-Fc (inert) | 150 | 20739 | 1.00 | 1618 | 1.00 | 1773 | 1.00 | 12.8 |
| WT CD80 ECD-Fc | 2 | 72506 | 3.50 | 3072 | 1.90 | 4418 | 2.49 | 23.6 |
| (inert) | | | | | | | | |

### Example 7

### Selection of Additional Variant CD80 IgV Domains and Assessment of Binding Activity

In order to refine affinity and functional potency of CD80 IgV variant interactions with counter structures CTLA-4, CD28 and PDL1, second and third generations (Gen) of random mutagenesis and selection were run using procedures substantially described in Examples 1-3. Briefly, yeast plasmid DNA was isolated from outgrown yeast post FACS selection and used as template for mutagenic PCR. To maximize diversity, both characterized individual variants and a pool of FACS selected variants were used as template. The resulting library was subjected to iterative rounds of FACS selection and outgrowth. To increase PDL1 affinity while maintaining CD28 affinity, multiple FASC sort progression paths were taken. The second-generation mutagenic library underwent four FACS selections alternating between CD28− and CTLA-4+ selections generating outputs that, when titrated against counter structures, were chosen to be reformatted into Fc vectors. The third-generation mutagenic library used the following FACS selection paths to yield yeast outputs that, when titrated against counter structures, were chosen to be reformatted into Fc vectors: 1. 50 nM PDL1+, 2a. 1 nM CTLA-4+, 2b. 20 nM CTLA-4−, 2a3. 10 nM PDL1+, 2b. 10 nM PDL1+, 2b34. 25 nM CD28+. Following selection of yeast expressing affinity modified variants of CD80, the selected variants were reformatted as Fc fusion for the generation of additional Fc-fusion proteins containing IgV CD80 variants. After sequence analysis, individual variants were chosen for protein production, binding and functional assay. Variants from generation 1 mutagenesis are shown in Table E1, generation 2 shown in Table E2, generation 3 shown in Tables E3 and E4.

Binding of selected immunomodulatory fusion proteins to cognate binding partners was assessed. To produce cells expressing the CD80 cognate binding partners, huCTLA4 and huPD-L1, full-length mammalian surface expression constructs were generated, incorporated into lentivirus and transduced into CHO cells. Cells were sorted in a Bio-Rad S3 Cell Sorter (Bio-Rad Corp., USA) to >98% purity. Jurkat/IL2 reporter cells, which endogenously express CD28, were used to detect binding to CD28.

For staining and analysis by flow cytometry, 100,000 cells of appropriate transfected cells were plated in 96-well round bottom plates. Cells were spun down and resuspended in staining buffer (phosphate buffered saline (PBS), 1% bovine serum albumin (BSA), and 0.1% sodium azide) for 20 minutes to block non-specific binding. Afterwards, cells were centrifuged and resuspended in staining buffer containing a six-point serial dilution (concentrations ranged from 100 nM to 41 pM) of each candidate variant CD80-Fc protein in 50 μl. Primary staining was performed on ice for 45 minutes, before washing cells in staining buffer twice. Phycoerythrin (PE)-conjugated anti-human Fc (Jackson ImmunoResearch, USA) was diluted 1:150, added to cells and incubated another 30 minutes on ice. Cells were then washed twice with 150 μL/well stain buffer, fixed in 2% formaldehyde/PBS, and analyzed on Intellicyt flow cytometer (Intellicyt Corp., USA). PE Mean Fluorescence Intensity (MFI) was calculated for each cell type with FlowJo Version 10 software (FlowJo LLC, USA).

Results for two binding studies for exemplary CD80 variants are shown in Tables E3 and E4. In the Tables, the exemplary amino acid substitutions are designated by amino acid position number corresponding to numbering of the respective reference unmodified IgV sequence. For example, the reference unmodified ECD sequence is the unmodified CD80 ECD sequence set forth in SEQ ID NO:2. The amino acid position is indicated in the middle, with the corresponding unmodified (e.g., wild-type) amino acid listed before the number and the identified variant amino acid substitution listed after the number. The second column sets forth the SEQ ID NO identifier for the variant IgV for each variant IgV-Fc fusion molecule.

Also shown is the binding activity as measured by the Mean Fluorescence Intensity (MFI) value for the binding of 33 nM of each variant CD80 Fc-fusion molecule to cells engineered to express the indicated cognate counter structure ligand (i.e., CTLA-4, PD-L1, or CD28) and the ratio of the MFI of the variant CD80 IgV-Fc, compared to the binding of the unmodified CD80-ECD-Fc fusion molecule (R&D Systems, USA) not containing the amino acid substitution(s), to the same cell-expressed counter structure ligand. The ratio of the binding of the variant CD80 IgV-Fc to the PD-L1 counter structure compared to the binding of the variant CD80 IgV-Fc to the CD28 counter structure also is shown in the last column of the Tables.

As shown, the selections resulted in the identification of several CD80 IgV domain variants that were affinity-modified to exhibit increased binding for PD-L and/or CD28 counter structures. Several variants also retained or exhibited increased binding to CTLA-4, while others exhibited decreased binding to CTLA-4. In addition, the results indicate that a number of variants were selected that exhibit reduced binding to CD28, including several CD80 IgV domain variants that exhibit increased binding to the PD-L1 counter structure ligand compared to the CD28 counter structure ligand (Ratio of PD-L1:CD28). Thus, the variants have unique profiles for binding cell-surface CTLA4, CD28, and PD-L1 as measured by flow cytometry.

TABLE E3
Variant CD80 Flow Binding to Jurkat Cells (CD28) and CHO cells stably expressing
CTLA4 or PD-L1

| | | CTLA4 | | CD28 | | PD-L1 | | Ratio |
|---|---|---|---|---|---|---|---|---|
| | SEQ | MFI at | Fold | MFI at | Fold | MFI at | Fold | of |
| | ID NO | 33.3 | change | 33.3 | change | 33.3 | change | PDL1: |
| CD80 mutation(s) | (IgV) | nM | to WT CD80 | nM | to WT | nM | to WT | CD28 |
| A26E/Q33R/E35D/M47L/ | 416 | 1275 | 0.01 | 275 | 0.04 | 75974 | 9.56 | 276 |
| L85Q/K86E | | | | | | | | |
| A26E/Q33R/E35D/M47L/ | 417 | 1280 | 0.01 | 264 | 0.03 | 81533 | 10.26 | 309 |
| L85Q | | | | | | | | |
| E35D/M47L/L85Q | 418 | 336179 | 1.88 | 646 | 0.08 | 33200 | 4.18 | 51 |
| A26E/Q33L/E35D/M47L/ | 419 | 1172 | 0.01 | 274 | 0.04 | 62680 | 7.89 | 229 |
| L85Q | | | | | | | | |
| A26E/Q33L/E35D/M47L | 420 | 1316 | 0.01 | 271 | 0.04 | 60903 | 7.67 | 225 |
| H18Y/A26E/Q33L/E35D/ | 421 | 2088 | 0.01 | 272 | 0.04 | 76591 | 9.64 | 282 |
| M47L/L85Q | | | | | | | | |
| Q33L/E35D/M47I | 422 | 15919 | 0.09 | 282 | 0.04 | 37353 | 4.70 | 132 |
| H18Y/Q33L/E35D/M47I | 423 | 5539 | 0.03 | 295 | 0.04 | 47793 | 6.02 | 162 |
| Q33L/E35D/D46E/M47I | 424 | 23328 | 0.13 | 281 | 0.04 | 42137 | 5.30 | 150 |
| Q33R/E35D/D46E/M47I | 425 | 3562 | 0.02 | 303 | 0.04 | 53345 | 6.72 | 176 |
| H18Y/E35D/M47L | 426 | 284445 | 1.59 | 5068 | 0.66 | 44161 | 5.56 | 9 |
| Q33L/E35D/M47V | 427 | 47648 | 0.27 | 281 | 0.04 | 47911 | 6.03 | 170 |
| Q33L/E35D/M47V/T79A | 428 | 28899 | 0.16 | 285 | 0.04 | 62078 | 7.82 | 218 |
| Q33L/E35D/T41S/M47V | 429 | 14515 | 0.08 | 287 | 0.04 | 43850 | 5.52 | 153 |
| Q33L/E35D/M47I/L85Q | 430 | 20548 | 0.11 | 287 | 0.04 | 63930 | 8.05 | 222 |
| Q33L/E35D/M47I/T62N/ | 431 | 1658 | 0.01 | 284 | 0.04 | 72578 | 9.14 | 256 |
| L85Q | | | | | | | | |
| Q33L/E35D/M47V/L85Q | 432 | 75368 | 0.42 | 268 | 0.04 | 47438 | 5.97 | 177 |
| A26E/E35D/M43T/M47L/ | 433 | 278021 | 1.56 | 260 | 0.03 | 68089 | 8.57 | 262 |
| L85Q/R94Q | | | | | | | | |
| Q33R/E35D/K37E/M47V/ | 434 | 22701 | 0.13 | 258 | 0.03 | 44438 | 5.59 | 172 |
| L85Q | | | | | | | | |
| V22A/E23D/Q33L/E35D/ | 435 | 3636 | 0.02 | 274 | 0.04 | 75513 | 9.51 | 275 |
| M47V | | | | | | | | |
| E24D/Q33L/E35D/M47V/ | 436 | 310964 | 1.74 | 3180 | 0.42 | 67066 | 8.44 | 21 |
| K54R/L85Q | | | | | | | | |
| S15P/Q33L/E35D/M47L/ | 437 | 22377 | 0.13 | 266 | 0.03 | 51558 | 6.49 | 194 |
| L85Q | | | | | | | | |
| E7D/E35D/M47I/L97Q | 438 | 270798 | 1.52 | 273 | 0.04 | 14643 | 1.84 | 54 |
| Q33L/E35D/T41S/M43I | 439 | 6388 | 0.04 | 433 | 0.06 | 44935 | 5.66 | 104 |
| E35D/M47I/K54R/L85E | 440 | 8665 | 0.05 | 285 | 0.04 | 36917 | 4.65 | 130 |
| Q33K/E35D/D46V/L85Q | 441 | 8507 | 0.05 | 257 | 0.03 | 26676 | 3.36 | 104 |
| Y31S/E35D/M47L/T79L/ | 442 | 1095 | 0.01 | 278 | 0.04 | 38909 | 4.90 | 140 |
| E88G | | | | | | | | |
| H18L/V22A/E35D/M47L/ | 443 | 373548 | 2.09 | 434 | 0.06 | 98110 | 12.35 | 226 |
| N48T/L85Q | | | | | | | | |
| Q27H/E35D/M47L/L85Q/ | 444 | 288596 | 1.61 | 282 | 0.04 | 36055 | 4.54 | 128 |
| R94Q/E95K | | | | | | | | |
| Q33K/E35D/M47V/K89E/ | 445 | 1752 | 0.01 | 276 | 0.04 | 39061 | 4.92 | 142 |
| K93R | | | | | | | | |
| E35D/M47I/E77A/L85Q/ | 446 | 247334 | 1.38 | 272 | 0.04 | 64521 | 8.12 | 238 |
| R94W | | | | | | | | |
| A26E/E35D/M43I/M47L/ | 447 | 2947 | 0.02 | 314 | 0.04 | 49440 | 6.22 | 157 |
| L85Q/K86E/R94W | | | | | | | | |
| Q27H/Q33L/E35D/M47V/ | 448 | 56061 | 0.31 | 269 | 0.04 | 14802 | 1.86 | 55 |
| N55D/L85Q/K89N | | | | | | | | |
| H18Y/V20A/Q33L/E35D/ | 449 | 2878 | 0.02 | 260 | 0.03 | 120517 | 15.17 | 463 |
| M47V/Y53F | | | | | | | | |
| V22A/E35D/V68E/A71D | 450 | 437038 | 2.45 | 13987 | 1.83 | 1350 | 0.17 | 0 |
| Q33L/E35D/M47L/A71G/ | 451 | 2107 | 0.01 | 366 | 0.05 | 28041 | 3.53 | 77 |
| F92S | | | | | | | | |
| V22A/R29H/E35D/D46E/ | 452 | 77423 | 0.43 | 323 | 0.04 | 25407 | 3.20 | 79 |
| M47I | | | | | | | | |
| Q33L/E35D/M43I/L85Q/ | 453 | 1083 | 0.01 | 272 | 0.04 | 29001 | 3.65 | 107 |
| R94W | | | | | | | | |
| H18Y/E35D/V68M/L97Q | 454 | 172538 | 0.97 | 299 | 0.04 | 121591 | 15.31 | 407 |
| Q33L/E35D/M47L/V68M/ | 455 | 3526 | 0.02 | 264 | 0.03 | 125741 | 15.83 | 476 |
| L85Q/E88D | | | | | | | | |
| Q33L/E35D/M43V/M47I/ | 456 | 13964 | 0.08 | 284 | 0.04 | 78029 | 9.82 | 275 |
| A71G | | | | | | | | |
| E35D/M47L/A71G/L97Q | 457 | 225591 | 1.26 | 300 | 0.04 | 65944 | 8.30 | 220 |
| E35D/M47V/A71G/L85M/ | 458 | 239089 | 1.34 | 339 | 0.04 | 61708 | 7.77 | 182 |
| L97Q | | | | | | | | |
| H18Y/Y31H/E35D/M47V/ | 459 | 3835 | 0.02 | 268 | 0.04 | 76364 | 9.61 | 285 |
| A71G/L85Q | | | | | | | | |
| E35D/D46E/M47V/L97Q | 460 | 305331 | 1.71 | 371 | 0.05 | 19484 | 2.45 | 52 |
| E35D/D46V/M47I/A71G/ | 461 | 287194 | 1.61 | 7543 | 0.99 | 45755 | 5.76 | 6 |
| F92V | | | | | | | | |
| E35D/M47V/T62A/A71G/ | 462 | 18113 | 0.10 | 305 | 0.04 | 77547 | 9.76 | 255 |
| V83A/Y87H/L97M | | | | | | | | |
| Q33L/E35D/N48K/L85Q/ | 463 | 1183 | 0.01 | 279 | 0.04 | 45185 | 5.69 | 162 |
| L97Q | | | | | | | | |
| WT CD80 ECD-Fc | 2 | 178708 | 1.00 | 7627 | 1.00 | 7943 | 1.00 | 1 |
| (R&D) | | | | | | | | |

TABLE E4
Variant CD80 Flow Binding to Jurkat Cells (CD28) and CHO cells stably expressing
CTLA4 or PD-L1

| | | CTLA4 | | CD28 | | PD-L1 | | Ratio |
|---|---|---|---|---|---|---|---|---|
| | SEQ | MFI at | Fold | MFI at | Fold | MFI at | Fold | of |
| | ID NO | 33.3 | change | 33.3 | change | 33.3 | change | PDL1: |
| CD80 mutation(s) | (IgV) | nM | to WT CD80 | nM | to WT CD80 | nM | to WT CD80 | CD28 |
| E35D/L85Q/K93I/E95V/ | 464 | 246401 | 1.57 | 400 | 0.02 | 19880 | 1.67 | 50 |
| L97Q | | | | | | | | |
| E35D/M47V/N48KN68M/ | 465 | 807 | 0.01 | 11736 | 0.65 | 89775 | 7.56 | 8 |
| K89N | | | | | | | | |
| Q33L/E35D/M47I/N48D/ | 466 | 116798 | 0.74 | 644 | 0.04 | 31151 | 2.62 | 48 |
| A71G | | | | | | | | |
| R29H/E35D/M43V/M47I/ | 467 | 4694 | 0.03 | 336 | 0.02 | 1590 | 0.13 | 5 |
| I49V | | | | | | | | |
| Q27H/E35D/M47I/L85Q/D90G | 468 | 257734 | 1.64 | 3513 | 0.19 | 30667 | 2.58 | 9 |
| E35D/M47I/L85Q/D90G | 469 | 247703 | 1.57 | 4095 | 0.23 | 35710 | 3.01 | 9 |
| E35D/M47I/T62S/L85Q | 470 | 300845 | 1.91 | 1758 | 0.10 | 44975 | 3.79 | 26 |
| A26E/E35D/M47L/A71G | 471 | 341248 | 2.17 | 2161 | 0.12 | 53352 | 4.49 | 25 |
| E35D/M47I/Y87Q/K89E | 472 | 110177 | 0.70 | 15452 | 0.86 | 29803 | 2.51 | 2 |
| V22A/E35D/M47I/Y87N | 473 | 245711 | 1.56 | 15299 | 0.85 | 35251 | 2.97 | 2 |
| H18Y/A26E/E35D/M47L/ | 474 | 230588 | 1.47 | 3540 | 0.20 | 52390 | 4.41 | 15 |
| L85Q/D90G | | | | | | | | |
| E35D/M47L/A71G/L85Q | 475 | 156254 | 0.99 | 1436 | 0.08 | 50474 | 4.25 | 35 |
| E35D/M47V/A71G/E88D | 476 | 211831 | 1.35 | 6237 | 0.35 | 37146 | 3.13 | 6 |
| E35D/A71G | 477 | 184204 | 1.17 | 4299 | 0.24 | 34149 | 2.88 | 8 |
| E35D/M47V/A71G | 478 | 226532 | 1.44 | 6360 | 0.35 | 36216 | 3.05 | 6 |
| I30V/E35D/M47V/A71G/ | 479 | 204756 | 1.30 | 5779 | 0.32 | 43877 | 3.70 | 8 |
| A91V | | | | | | | | |
| V22D/E35D/M47L/L85Q | 481 | 256426 | 1.63 | 542 | 0.03 | 34908 | 2.94 | 64 |
| H18Y/E35D/N48K | 482 | 260795 | 1.66 | 4189 | 0.23 | 45849 | 3.86 | 11 |
| E35D/T41S/M47V/A71G/ | 483 | 251238 | 1.60 | 5314 | 0.29 | 45436 | 3.83 | 9 |
| K89N | | | | | | | | |
| E35D/M47V/N48I/L85Q | 484 | 281417 | 1.79 | 692 | 0.04 | 35491 | 2.99 | 51 |
| E35D/D46E/M47V/A71D/ | 485 | 274661 | 1.75 | 6169 | 0.34 | 32371 | 2.73 | 5 |
| D90G | | | | | | | | |
| E35D/D46E/M47V/A71D | 486 | 174016 | 1.11 | 5949 | 0.33 | 549 | 0.05 | 0 |
| E35D/T41S/M43I/A71G/ | 487 | 208017 | 1.32 | 9249 | 0.51 | 56172 | 4.73 | 6 |
| D90G | | | | | | | | |
| E35D/T41S/M43I/M47V/ | 488 | 243502 | 1.55 | 2845 | 0.16 | 44419 | 3.74 | 16 |
| A71G | | | | | | | | |
| E35D/T41S/M43I/M47L/ | 489 | 209034 | 1.33 | 3104 | 0.17 | 59613 | 5.02 | 19 |
| A71G | | | | | | | | |
| H18Y/V22A/E35D/M47V/ | 490 | 219782 | 1.40 | 4214 | 0.23 | 87702 | 7.39 | 21 |
| T62S/A71G | | | | | | | | |
| H18Y/A26E/E35D/M47L/ | 491 | 253787 | 1.61 | 14934 | 0.83 | 170935 | 14.40 | 11 |
| V68M/A71G/D90G | | | | | | | | |
| E35D/K37E/M47V/N48D/ | 492 | 243506 | 1.55 | 1589 | 0.09 | 26542 | 2.24 | 17 |
| L85Q/D90N | | | | | | | | |
| Q27H/E35D/D46V/M47L/ | 493 | 157358 | 1.00 | 10412 | 0.58 | 60139 | 5.07 | 6 |
| A71G | | | | | | | | |
| V22L/Q27H/E35D/M47I/ | 494 | 151600 | 0.96 | 7269 | 0.40 | 43797 | 3.69 | 6 |
| A71G | | | | | | | | |
| E35D/D46V/M47LN68M/ | 495 | 224734 | 1.43 | 5027 | 0.28 | 137368 | 11.57 | 27 |
| L85Q/E88D | | | | | | | | |
| E35D/T41S/M43V/M47I/ | 496 | 249456 | 1.59 | 2698 | 0.15 | 12978 | 1.09 | 5 |
| L70M/A71G | | | | | | | | |
| E35D/D46E/M47V/N63D/ | 497 | 274320 | 1.74 | 1331 | 0.07 | 69780 | 5.88 | 52 |
| L85Q | | | | | | | | |
| E35D/M47V/T62A/A71D/ | 498 | 225737 | 1.44 | 12030 | 0.67 | 693 | 0.06 | 0 |
| K93E | | | | | | | | |
| E35D/D46E/M47V/V68M/ | 499 | 273157 | 1.74 | 27080 | 1.50 | 71903 | 6.06 | 3 |
| D90G/K93E | | | | | | | | |
| E35D/M43I/M47V/K89N | 500 | 278391 | 1.77 | 6752 | 0.37 | 19250 | 1.62 | 3 |
| E35D/M47L/A71G/L85M/ | 501 | 215998 | 1.37 | 2459 | 0.14 | 46684 | 3.93 | 19 |
| F92Y | | | | | | | | |
| E35D/M42V/M47V/E52D/ | 502 | 225986 | 1.44 | 1291 | 0.07 | 11897 | 1.00 | 9 |
| L85Q | | | | | | | | |
| V22D/E35D/M47L/L70M/ | 503 | 127835 | 0.81 | 527 | 0.03 | 17670 | 1.49 | 34 |
| L97Q | | | | | | | | |
| E35D/T41S/M47V/L97Q | 504 | 262204 | 1.67 | 290 | 0.02 | 13591 | 1.14 | 47 |
| E35D/Y53H/A71G/D90G/ | 505 | 182701 | 1.16 | 1547 | 0.09 | 57455 | 4.84 | 37 |
| L97R | | | | | | | | |
| E35D/A71D/L72V/R73H/ | 506 | 186582 | 1.19 | 3365 | 0.19 | 503 | 0.04 | 0 |
| E81K | | | | | | | | |
| Q33L/E35D/M43I/Y53F/ | 507 | 3985 | 0.03 | 1024 | 0.06 | 72065 | 6.07 | 70 |
| T62S/L85Q | | | | | | | | |
| E35D/M38T/D46E/M47V/ | 508 | 175387 | 1.11 | 587 | 0.03 | 19393 | 1.63 | 33 |
| N48S | | | | | | | | |
| Q33R/E35D/M47V/N48K/ | 509 | 2680 | 0.02 | 265 | 0.01 | 21425 | 1.80 | 81 |
| L85M/F92L | | | | | | | | |
| E35D/M38I/M43V/M47V/ | 510 | 203938 | 1.30 | 285 | 0.02 | 21795 | 1.84 | 76 |
| N48R/L85Q | | | | | | | | |
| I28Y/Q33H/E35D/D46V/ | 511 | 156810 | 1.00 | 298 | 0.02 | 46038 | 3.88 | 154 |
| M47I/A71G | | | | | | | | |
| WT CD80 ECD-Fc | 2 | 157306 | 1.00 | 18035 | 1.00 | 11871 | 1.00 | 1 |
| (R&D) | | | | | | | | |

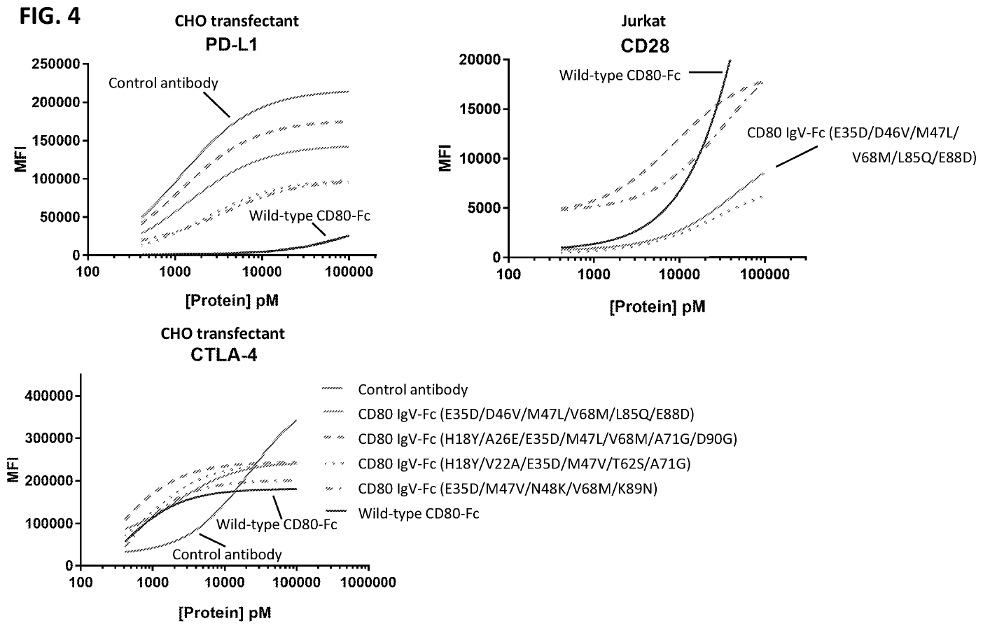

To further compare binding, various concentrations of exemplary variant CD80 IgV-Fc molecules were assessed and compared to wild-type CD80 IgV-Fc for binding to cell surface expressed PD-L1, CD28 and CTLA-4. The exemplary tested variant CD80 IgV-Fc included: E35D/D46V/M47L/V68M/L85Q/E88D (SEQ ID NO: 495), H18Y/A26E/E35D/M47L/V68M/A71G/D90G (SEQ ID NO: 491), H18Y/V22A/E35D/M47V/T62S/A71G (SEQ ID NO: 490), and E35D/M47V/N48K/V68M/K89N (SEQ ID NO: 465). Binding to CD28 was assessed using Jurkat/IL2 reporter cells expressing CD28 and binding to CTLA-4 and PD-L1 was assessed using CHO cells stably transfected to express huCTLA-4 or huPD-L1 as described above. Indicated transfectants or cell lines were plated and stained with titrated amounts of CD80 vIgD-Fc or wild-type CD80 IgV-Fc. Bound protein was detected with fluorochrome conjugated anti-huFc and Mean Fluorescence Intensity (MFI) measured by flow cytometry. As shown in FIG. 4, some tested CD80 vIgD-Fc bound human PD-L1, human CTLA-4, and human CD28 with higher affinity than wild-type CD80.

### Example 8

### Assessment of Bioactivity of Affinity-Matured CD80 IgSF Domain-Containing Molecules Using a Jurkat/IL2 Reporter Assay

This Example describes a Jurkat/IL2 reporter assay to assess bioactivity of CD80 domain variant immunomodulatory proteins for blockade of CD28 costimulation.

The day before the assay, the assay plate was prepared. To prepare the assay plate, 10 nM anti-CD3 antibody (clone OKT3; BioLegend, catalog no. 317315) and 20 nM CD86-Fc (R&D Systems, catalog no. 141-B2) in PBS were aliquoted at 100 μL/well into a white, flat-bottom 96-well plate (Costar). The plate was incubated overnight at 4° C. to allow the antibody and CD86-Fc protein to adhere to the surface of the plate. The next day, the wells of the assay plate were washed twice with 150 μL PBS prior to the assay.

The day of the assay, 60 μL exemplary variant CD80 IgV-Fc fusion molecules and control, wildtype CD80 IgV-Fc or wildtype CD80 (ECD)-Fc, molecules, or negative control Fc alone, were diluted to a concentration of 40 nM in assay buffer (RPMI1640+5% fetal bovine serum (FBS)), or buffer alone, and were added to the wells of a fresh 96-well polypropylene plate. Jurkat effector cells expressing IL-2-luciferase reporter were counted and resuspended in assay buffer to a concentration of 2×10⁶ cells/mL. 60 μL of the Jurkat cell suspension were then added to the wells containing the CD80-Fc fusion molecules or controls. The cells and CD80 proteins were incubated at room temperature for 15 minutes and then 100 μL of the cell/CD80 protein mixture were transferred/well of the prepared anti-CD3/CD86-Fc assay plate.

The assay plate was briefly spun down (10 seconds at 1200 RPM) and incubated at 37° C. for 5 hours. After the 5 hour incubation, the plate was removed and equilibrated to room temperature for 15 minutes. 100 μL of Bio-Glo (Promega) were added/well of the assay plate, which was then placed on an orbital shaker for 10 minutes. Luminescence was measured with a 1 second per well integration time using a BioTek Cytation 3 luminometer.

An average relative luminescence value was determined for each variant CD80 IgV Fc and a fold increase in IL-2 reporter signal was calculated for each variant compared to wildtype CD80 IgV-Fc protein. The results are provided in Table E5 below.

As shown in Table E5, co-culturing many of the exemplary variant CD80 IgV-Fc molecules with Jurkat effector cells expressing IL-2-luciferase reporter, resulted in decreased CD28 costimulation (i.e., blockade) compared to buffer only or the Fc-only negative control. Several of the variant CD80 IgV-Fc molecules appeared to increase the CD28 costimulatory signal compared to the wild-type CD80 IgV-Fc molecule suggesting possible agonistic activity.

TABLE E5
Jurkat/IL2 Reporter Assay: Blockade of
CD28 Costimulation

| | | Average | |
|---|---|---|---|
| | | Relative | Fold increase |
| CD80 Mutation(s) | SEQ ID NO (IgV) | Luminescence Units | in IL2 reporter signal |
| Q27H/T41S/A71D | 153 | 1301 | 0.32 |
| I30T/L70R | 154 | 3236 | 0.79 |
| T13R/C16R/L70Q/A71D | 155 | 3204 | 0.78 |
| T57I | 156 | 1463 | 0.36 |
| M43I/C82R | 157 | 1326 | 0.32 |
| V22L/M38V/M47T/A71D/L85M | 158 | 1770 | 0.43 |
| I30V/T57I/L70P/A71D/A91T | 159 | 1731 | 0.42 |
| V22I/L70M/A71D | 160 | 253 | 0.06 |
| N55D/K86M | 163 | 4277 | 1.04 |
| L72P/I79I | 164 | 4157 | 1.01 |
| L70P/F92S | 165 | 5035 | 1.22 |
| T79P | 166 | 4397 | 1.07 |
| E35D/M47I/L65P/D90N | 167 | 2377 | 0.58 |
| L25S/E35D/M47I/D90N | 168 | 2567 | 0.62 |
| A71D | 170 | 999 | 0.24 |
| E81K/A91S | 172 | 4038 | 0.98 |
| A12V/M47V/L70M | 173 | 4999 | 1.22 |
| K34E/T41A/L72V | 174 | 4225 | 1.03 |
| T41S/A71D/V84A | 175 | 2685 | 0.65 |
| E35D/A71D | 176 | 1461 | 0.36 |
| E35D/M47I | 177 | 1444 | 0.35 |
| K36R/G78A | 178 | 2597 | 0.63 |
| Q33E/T41A | 179 | 4220 | 1.03 |
| M47V/N48H | 180 | 2656 | 0.65 |
| M47L/V68A | 181 | 5445 | 1.32 |
| S44P/A71D | 182 | 2848 | 0.69 |
| Q27H/M43I/A71D/R73S | 183 | 1891 | 0.46 |
| E35D/T57I/L70Q/A71D | 185 | 280 | 0.07 |
| M47I/E88D | 186 | 2178 | 0.53 |
| M42I/I61V/A71D | 187 | 2549 | 0.62 |
| P51A/A71D | 188 | 4690 | 1.14 |
| H18Y/M47I/T57I/A71G | 189 | 924 | 0.22 |
| V20I/M47V/T57I/V84I | 190 | 1870 | 0.45 |
| V20I/M47V/A71D | 191 | 360 | 0.09 |
| A71D/L72V/E95K | 192 | 2939 | 0.71 |
| V22L/E35G/A71D/L72P | 193 | 2334 | 0.57 |
| E35D/A71D | 194 | 812 | 0.20 |
| E35D/I67L/A71D | 195 | 1223 | 0.30 |
| T13R/M42V/M47I/A71D | 197 | 759 | 0.18 |
| E35D | 198 | 1981 | 0.48 |
| E35D/M47I/L70M | 199 | 1077 | 0.26 |
| E35D/A7I/L72V | 200 | 1152 | 0.28 |
| E35D/M43L/L70M | 201 | 3640 | 0.88 |
| A26P/E35D/M43I/L85Q/E88D | 202 | 4078 | 0.99 |
| E35D/D46V/L85Q | 203 | 3230 | 0.79 |
| Q27L/E35D/M47I/T57I/L70Q/E88D | 204 | 1180 | 0.29 |
| Q27H/E35G/A71D/L72P/T791 | 196 | 2000 | 0.49 |
| M47V/169F/A71D/V831 | 205 | 290 | 0.07 |
| E35D/T57A/A71D/L85Q | 206 | 3213 | 0.78 |
| H18Y/A26T/E35D/A71D/L85Q | 207 | 2773 | 0.67 |
| E35D/M47L | 208 | 1110 | 0.27 |
| E23D/M42V/M43I/I58V/L70R | 209 | 4460 | 1.08 |
| V68M/L70M/A71D/E95K | 210 | 2067 | 0.50 |
| N55I/T57I/I69F | 211 | 1915 | 0.47 |
| E35D/M43I/A71D | 212 | 3019 | 0.73 |
| T41S/T57I/L70R | 213 | 3641 | 0.89 |
| H18Y/A71D/L72P/E88V | 214 | 1354 | 0.33 |
| V20I/A71D | 215 | 2165 | 0.53 |
| E23G/A26S/E35D/T62N/A71D/ | 216 | 2067 | 0.50 |
| L72V/L85M | | | |
| A12T/E24D/E35D/D46V/161V/ | 217 | 2408 | 0.59 |
| L72P/E95V | | | |
| V22L/E35D/M43L/A71G/D76H | 218 | 2004 | 0.49 |
| E35G/K54E/A71D/L72P | 219 | 3618 | 0.88 |
| L70Q/A71D | 220 | 1036 | 0.25 |
| A26E/E35D/M47L/L85Q | 221 | 4111 | 1.00 |
| D46E/A71D | 222 | 490 | 0.12 |
| Y31H/E35D/T41S/V68L/K93R/R94W | 223 | 3678 | 0.89 |
| WT CD80 IgV-Fc | 150 | 4113 | 1.00 |
| WT CD80 ECD-Fc | 2 | 3816 | 0.93 |
| Fc only Control | — | 4107 | 1.00 |
| Buffer Only | — | 4173.25 | 1.01 |

### Example 9

### Assessment of Bioactivity of Affinity-Matured CD80 IgSF Domain-Containing Molecules in the Presence and Absence of PD-L1 Using a Jurkat/IL2 Reporter Assay

This Example describes a Jurkat/IL2 reporter assay to assess the capacity of CD80 domain variant immunomodulatory proteins fused to either an inert Fc molecule (e.g. SEQ ID NO: 1520, or allotypes thereof) or an Fc molecule capable of mediating effector activity (SEQ ID NO: 1517) to modulate CD28 costimulation signal in the presence or absence of PD-L1-expressing antigen presenting cells.

A. PD-L1-Dependent CD28 Costimulation

Jurkat effector cells expressing an IL-2-luciferase reporter (purchased from Promega Corp., USA) were suspended at 2×10⁶ cells/mL in Jurkat Assay buffer (RPMI1640+5% FBS). Jurkat cells were then plated at 50 μL/well for a total of 100,000 cells per well.

To each well, 25 μL of test protein was added to the Jurkat cells. Test proteins included variant CD80 IgV-Fc (inert) fusion molecules or full CD80-ECD-Fc (R&D Systems, USA) or wild type CD80-IgV-Fc (inert). All proteins were added at: 200 nM, 66.7 nM, and 22.2 nM (no PD-L1) or 200 nM, 66.7 nM, 22.2 nM, 7.4 nM, and 2.5 nM (+PD-L1). The Jurkat cells with test or control proteins were incubated for 15 minutes at room temperature. CHO-derived artificial antigen presenting cells (aAPC) displaying transduced cell surface anti-CD3 single chain Fv (OKT3) (i.e., no PD-L1), or OKT3 and PD-L1 (i.e., +PD-L1), were brought to 0.8×10⁶ cells/mL, and 25 μL of cells were added to each well, bringing the final volume of each well to 100 μL. Each well had a final ratio of 5:1 Jurkat:CHO cells and a test protein concentration of 50, 16.7 or 5.6 nM (no PD-L1), or 50, 16.7, 5.6, 1.9, and 0.6 nM (+PD-L1). Jurkat cells and CHO cells were incubated for 5 hours at 37 degrees Celsius in a humidified 5% CO₂ incubation chamber. Plates were then removed from the incubator and acclimated to room temperature for 15 minutes. 100 μL of a cell lysis and luciferase substrate solution (BioGlo luciferase reagent, Promega) were added to each well and the plates were incubated on an orbital shaker for 10 minutes. Luminescence was measured with a 1 second per well integration time using a BioTek Cytation luminometer, and a relative luminescence value (RLU) was determined for each test sample. The results are provided in Table E6.

In the absence of PD-L1 on the aAPC, little to no co-stimulatory signal was observed consistent with the observation that variant CD80 molecules fused to an inert Fc were not able to induce a costimulatory signal via CD28. In the presence of PD-L1, however, several of the variant CD80-IgV-Fc (inert) molecules tested exhibited concentration dependent CD28 costimulation that was correlated with the CD28 and/or PD-L1 binding affinity of the variant molecules. This result indicates that variant CD80 molecules with increased affinity to PD-L1 are able to mediate PD-L1-dependent costimulation of CD28.

TABLE E6
PD-L1-Dependent CD28 Costimulation

| | | No PD-L1 | | | +PD-L1 | | | | |
|---|---|---|---|---|---|---|---|---|---|
| | SEQ ID | 5.6 | | 50 | 0.6 | | 5.6 | 16.7 | 50 |
| CD80 Mutation(s) | NO (IgV) | nM | 16.7 nM | nM | nM | 1.9 nM | nM | nM | nM |
| E35D/M47I | 177 | 637 | 710 | 894 | 1047 | 1732 | 2794 | 3672 | 3778 |
| A71D/L72V/E95K | 192 | 466 | 547 | 644 | 524 | 530 | 617 | 641 | 755 |
| E35D | 198 | 412 | 480 | 448 | 456 | 465 | 625 | 995 | 1606 |
| E35D/M47I/L70M | 199 | 549 | 544 | 600 | 1004 | 1640 | 2348 | 2629 | 2629 |
| E35D/M43L/L70M | 201 | 396 | 439 | 515 | 479 | 525 | 683 | 1066 | 1809 |
| E35D/D46V/L85Q | 203 | 511 | 554 | 720 | 611 | 1001 | 1486 | 1814 | 2224 |
| H18Y/A26I/E35D/ | 207 | 638 | 660 | 926 | 628 | 621 | 795 | 974 | 1156 |
| A71D/L85Q | | | | | | | | | |
| E35D/M47L | 208 | 633 | 731 | 817 | 1041 | 1730 | 2580 | 3069 | 2906 |
| E23G/A26S/E35D/ | 216 | 566 | 560 | 606 | 524 | 604 | 659 | 689 | 695 |
| T62N/A71D/ | | | | | | | | | |
| L72V/L85M | | | | | | | | | |
| E35G/K54E/A71D/L72P | 219 | 417 | 475 | 440 | 529 | 489 | 554 | 504 | 476 |
| A26E/E35D/M47L/L85Q | 221 | 458 | 415 | 432 | 509 | 618 | 886 | 1385 | 1998 |
| WT CD80 IgV-Fc (inert) | 150 | 450 | 444 | 479 | 458 | 486 | 511 | 523 | 483 |
| WT CD80 ECD-Fc (inert) | 2 | 436 | 412 | 420 | 518 | 474 | 505 | 462 | 449 |
| Fc only Control | — | 419 | 406 | 395 | 501 | 457 | 438 | 451 | 440 |

In a further experiment, other variant CD80 IgV-Fc (inert) fusion proteins were tested for CD28 stimulation in the absence of aAPCs+/−PD-L as described above, except the final concentrations of each test protein were 50 nM and 5 nM. A relative luminescence value (RLU) was determined for each test sample and a fold increase (or decrease) in EL-2 reporter signal was calculated for each variant CD80-IgV molecule and compared to wildtype CD80-ECD-Fc (inert) and CD80-IgV-Fc (inert) proteins.

As shown in Tables E7 and E8, the luciferase activity of the Jurkat effector cells co-cultured with K562/OKT3/PD-L1 aAPC and 50 nM CD8N-IgV-Fc (inert) molecules was altered (increased or decreased) for several of the molecules tested. Simultaneous binding of PD-L1 on the aAPC and CD28 on the Jurkat cell resulted in increased CD28-costimulation and downstream EL-2 signal transduction. Fold increase (or decrease) in luminescence relative to wildtype CD80-IgV-Fc (inert) is also shown. In the Table, the first column sets forth the mutation(s), and the second column sets forth the SEQ ID NO identifier for each CD80-IgV of a CD80-IgV Fc (inert) variant tested.

TABLE E7
Jurkat/IL2 + K562/OKT3/PD-L1 Reporter Assay:
Relative Luciferase Units (RLU)

| | | CD80-Fc | Fold Increase |
|---|---|---|---|
| CD80 Mutation(s) | SEQ ID NO (IgV) | Conc. 50 nM | over WT CD80-IgV-Fc |
| A26E/Q33R/E35D/M47L/L85Q/K86E | 416 | 569 | 1.0 |
| A26E/Q33R/E35D/M47L/L85Q | 417 | 500 | 0.9 |
| E35D/M47L/L85Q | 418 | 2852 | 5.0 |
| A26E/Q33L/E35D/M47L/L85Q | 419 | 416 | 0.7 |
| A26E/Q33L/E35D/M47L | 420 | 476 | 0.8 |
| H18Y/A26E/Q33L/E35D/M47L/L85Q | 421 | 408 | 0.7 |
| Q33L/E35D/M47I | 422 | 423 | 0.7 |
| H18Y/Q33L/E35D/M47I | 423 | 486 | 0.9 |
| Q33L/E35D/D46E/M47I | 424 | 554 | 1.0 |
| Q33R/E35D/D46E/M47I | 425 | 522 | 0.9 |
| H18Y/E35D/M47L | 426 | 2976 | 5.3 |
| Q33L/E35D/M47V | 427 | 393 | 0.7 |
| Q33L/E35D/M47V/T79A | 428 | 527 | 0.9 |
| Q33L/E35D/T41S/M47V | 429 | 481 | 0.8 |
| Q33L/E35D/M47I/L85Q | 430 | 432 | 0.8 |
| Q33L/E35D/M47I/T62N/L85Q | 431 | 463 | 0.8 |
| Q33L/E35D/M47V/L85Q | 432 | 556 | 1.0 |
| A26E/E35D/M43T/M47L/L85Q/R94Q | 433 | 526 | 0.9 |
| Q33R/E35D/K37E/M47V/L85Q | 434 | 464 | 0.8 |
| V22A/E23D/Q33L/E35D/M47V | 435 | 390 | 0.7 |
| E24D/Q33L/E35D/M47V/K54R/L85Q | 436 | 3235 | 5.7 |
| S15P/Q33L/E35D/M47L/L85Q | 437 | 468 | 0.8 |
| E7D/E35D/M47I/L97Q | 438 | 1243 | 2.2 |
| Q33L/E35D/T41S/M43I | 439 | 533 | 0.9 |
| E35D/M47I/K54R/L85E | 440 | 602 | 1.1 |
| Q33K/E35D/D46V/L85Q | 441 | 504 | 0.9 |
| Y31S/E35D/M47L/T79L/E88G | 442 | 496 | 0.9 |
| H18L/V22A/E35D/M47L/N48T/L85Q | 443 | 2652 | 4.7 |
| Q27H/E35D/M47L/L85Q/R94Q/E95K | 444 | 513 | 0.9 |
| Q33K/E35D/M47V/K89E/K93R | 445 | 415 | 0.7 |
| E35D/M47I/E77A/L85Q/R94W | 446 | 473 | 0.8 |
| A26E/E35D/M43I/M47L/L85Q/K86E/R94W | 447 | 498 | 0.9 |
| Q27H/Q33L/E35D/M47V/N55D/L85Q/K89N | 448 | 551 | 1.0 |
| H18Y/V20A/Q33L/E35D/M47V/Y53F | 449 | 566 | 1.0 |
| V22A/E35D/V68E/A71D | 450 | 538 | 1.0 |
| Q33L/E35D/M47L/A71G/F925 | 451 | 394 | 0.7 |
| V22A/R29H/E35D/D46E/M47I | 452 | 3314 | 5.9 |
| Q33L/E35D/M43I/L85Q/R94W | 453 | 553 | 1.0 |
| H18Y/E35DN68M/L97Q | 454 | 4336 | 7.7 |
| Q33L/E35D/M47L/V68M/L85Q/E88D | 455 | 572 | 1.0 |
| Q33L/E35D/M43V/M47I/A71G | 456 | 473 | 0.8 |
| E35D/M47L/A71G/L97Q | 457 | 2156 | 3.8 |
| E35D/M47V/A71G/L85M/L97Q | 458 | 576 | 1.0 |
| H18Y/Y31H/E35D/M47V/A71G/L85Q | 459 | 455 | 0.8 |
| E35D/D46E/M47V/L97Q | 460 | 1087 | 1.9 |
| E35D/D46V/M47I/A71G/F92V | 461 | 2254 | 4.0 |
| E35D/M47V/T62A/A71G/V83A/Y87H/L97M | 462 | 438 | 0.8 |
| Q33L/E35D/N48K/L85Q/L97Q | 463 | 358 | 0.6 |
| WT CD80-ECD-Fc (effector) | 2 | 3045 | 5.4 |
| WT CD80 | 150 | 566 | 1 |
| IgV-Fc (inert) | | | |

TABLE E8
Jurkat/IL2 + K562/OKT3/PD-L1 Reporter Assay:
Relative Luciferase Units (RLU)

| | | CD80-Fe | Fold Increase |
|---|---|---|---|
| CD80 Mutation(s) | SEQ ID NO (IgV) | Cone 50 nM | over WT CD80-IgV-Fe |
| E35D/L85Q/K93T/E95V/L97Q | 464 | 315 | 1.5 |
| E35D/M47V/N48K/V68M/K89N | 465 | 1439 | 7.0 |
| Q33L/E35D/M47I/N48D/A71G | 466 | 213 | 1.0 |
| R29H/E35D/M43V/M47I/I49V | 467 | 227 | 1.1 |
| Q27H/E35D/M47I/L85Q/D90G | 468 | 1313 | 6.4 |
| E35D/M47I/L85Q/D90G | 469 | 1438 | 7.0 |
| E35D/M47I/T62S/L85Q | 470 | 1571 | 7.6 |
| A26E/E35D/M47L/A71G | 471 | 1748 | 8.5 |
| E35D/M47I/Y87Q/K89E | 472 | 1581 | 7.7 |
| V22A/E35D/M47I/Y87N | 473 | 1388 | 6.7 |
| H18Y/A26E/E35D/M47L/L85Q/D90G | 474 | 1506 | 7.3 |
| E35D/M47L/A71G/L85Q | 475 | 1256 | 6.1 |
| E35D/M47V/A71G/E88D | 476 | 1216 | 5.9 |
| E35D/A71G | 477 | 1190 | 5.8 |
| E35D/M47V/A71G | 478 | 1190 | 5.8 |
| I30V/E35D/M47V/A71G/A91V | 479 | 1503 | 7.3 |
| V22D/E35D/M47L/L85Q | 481 | 1142 | 5.5 |
| H18Y/E35D/N48K | 482 | 1230 | 6.0 |
| E35D/T41S/M47V/A71G/K89N | 483 | 1023 | 5.0 |
| E35D/M47V/N48T/L85Q | 484 | 897 | 4.4 |
| E35D/D46E/M47V/A71D/D90G | 485 | 1042 | 5.1 |
| E35D/D46E/M47V/A71D | 486 | 683 | 3.3 |
| E35D/T41S/M43I/A71G/D90G | 487 | 1122 | 5.4 |
| E35D/T41S/M43I/M47V/A71G | 488 | 1273 | 6.2 |
| E35D/T41S/M43I/M47L/A71G | 489 | 1535 | 7.5 |
| H18Y/V22A/E35D/M47V/T62S/A71G | 490 | 1379 | 6.7 |
| H18Y/A26E/E35D/M47L/V68M/A71G/D90G | 491 | 1116 | 5.4 |
| E35D/K37E/M47V/N48D/L85Q/D90N | 492 | 851 | 4.1 |
| Q27H/E35D/D46V/M47L/A71G | 493 | 978 | 4.7 |
| V22L/Q27H/E35D/M47I/A71G | 494 | 1123 | 5.5 |
| E35D/D46V/M47L/V68M/L85Q/E88D | 495 | 1464 | 7.1 |
| E35D/T41S/M43V/M47I/L70M/A71G | 496 | 1672 | 8.1 |
| E35D/D46E/M47V/N63D/L85Q | 497 | 1381 | 6.7 |
| E35D/M47V/T62A/A71D/K93E | 498 | 1056 | 5.1 |
| E35D/D46E/M47V/V68M/D90G/K93E | 499 | 1261 | 6.1 |
| E35D/M43I/M47V/K89N | 500 | 1094 | 5.3 |
| E35D/M47L/A71G/L85M/F92Y | 501 | 1322 | 6.4 |
| E35D/M42V/M47V/E52D/L85Q | 502 | 1260 | 6.1 |
| V22D/E35D/M47L/L70M/L97Q | 503 | 1542 | 7.5 |
| E35D/T41S/M47V/L97Q | 504 | 594 | 2.9 |
| E35D/Y53H/A71G/D90G/L97R | 505 | 1723 | 8.4 |
| E35D/A71D/L72V/R73H/E81K | 506 | 282 | 1.4 |
| Q33L/E35D/M43I/Y53F/T62S/L85Q | 507 | 168 | 0.8 |
| E35D/M38T/D46E/M47V/N48S | 508 | 1315 | 6.4 |
| Q33R/E35D/M47V/N48K/L85M/F92L | 509 | 215 | 1.0 |
| E35D/M38T/M43V/M47V/N48R/L85Q | 510 | 680 | 3.3 |
| T28Y/Q33H/E35D/D46V/M47I/A71G | 511 | 580 | 2.8 |
| WT CD80 ECD-Fc (effector) | 2 | 1786 | 8.7 |
| WT CD80-IgV-Fc (inert) | 150 | 206 | 1.0 |

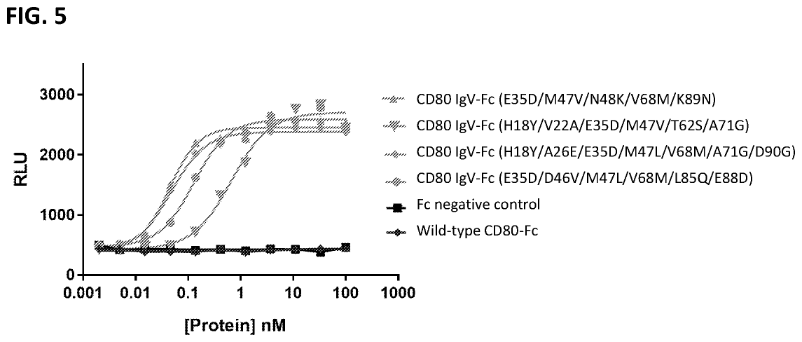

To further compare activity, various concentrations of exemplary variant CD80 IgV-Fc (inert) were assessed for induction of luciferase activity in Jurkat/IL2 reporter cells using the K562/OKT3/PDL1 aAPC cell line described above and activity was compared to wildtype CD80 IgV-Fc (inert). The exemplary variant CD80 IgV molecules that were tested contained E35D/M47V/N48K/V68M/K89N (SEQ ID NO: 465), H18Y/Y22A/E35D/M47V/T62S/A71G (SEQ ID NO: 490), H18Y/A26E/E35D/M47L/V68M/A71G/D90G (SEQ ID NO: 491), and E35D/D46V/M47L/V68M/L85Q/E88D (SEQ ID NO: 495). As shown in FIG. 5, the exemplary tested variant CD80 IgV domain-containing molecules induced PD-L1 dependent CD28 costimulation in a dose-dependent manner. No PD-L1 dependent CD28 costimulation was observed by wildtype CD80 IgV-Fc at any of the assessed concentrations.

B. Cytokine Production Following PD-L1-Dependent Costimulation

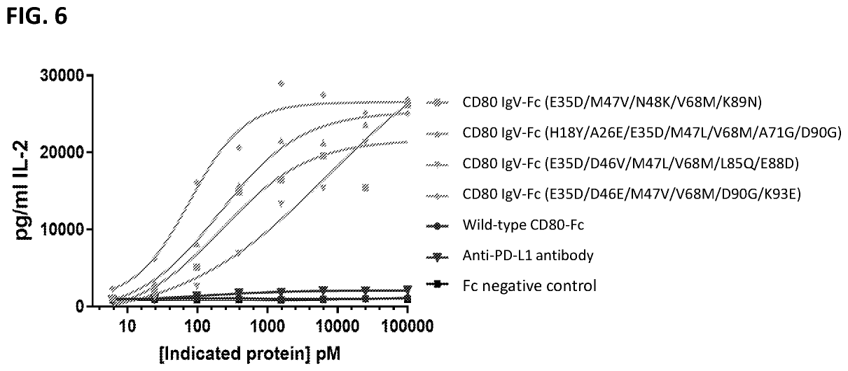

K562/OKT3/PDL1 aAPC cells described above were treated with mitomycin-c and co-cultured with primary human pan T cells in the presence of titrated increasing concentrations of CD80 IgV-Fc (inert) or wildtype CD80 IgV-Fc (inert). Exemplary variant CD80-Fcs tested contained E35D/M47V/N48K/V68M/K89N (SEQ ID NO: 465), H18Y/A26E/E35D/M47L/V68M/A71G/D90G (SEQ ID NO: 491), E35D/D46V/M47L/V68M/L85Q/E88D (SEQ ID NO: 495), E35D/D46E/M47V/V68M/D90G/K93E (SEQ ID NO: 499). As a further control, primary human pan T cells also were cultured with the exemplary anti-PD-L1 durvalumab or an Fc (inert) only control. Results, set forth in FIG. 6, showed that the tested variant CD80-IgV-Fc molecules resulted in IL-2 secretion in culture supernatants, consistent with an observation that PD-L1 dependent co-stimulation was induced by the tested exemplary variant CD80-IgV-Fc molecules. IL-2 production was not observed in T cell cultures when incubated with wildtype CD80-IgV Fc or other tested controls.

C. Fc-Dependent CD28 Costimulation +/−PD-L1

In a further experiment, CD28 costimulation was assessed for variant CD80-IgV-Fc fusion proteins, where the Fc was an IgG1 Fc (e.g. SEQ ID NO:1517) capable of mediating effector activity via binding to Fc receptors (FcR). The experiment was carried out as described in part A above, except CD32-expressing K562 cells stably transduced with OKT3 (K562/OKT3) or OKT3 and PD-L1 (K562/OKT3/PD-L1) were used instead of the CHO/OKT3 and CHO/OKT3/PD-L1 cells, and the results are depicted in Table E9.

TABLE E9
CD28 Costimulation via Fc Receptor or PD-L1 Dependent Cross-Linking

| | | K562/0KT3 aAPC | | | | | K562/0KT3/PD-L1 aAPC | | | | |
|---|---|---|---|---|---|---|---|---|---|---|---|
| | SEQ | FcR Dependent Cross-Linking | | | | | Combination of FcR and/or PD-L1 | | | | |
| | ID | (No PD-L1) | | | | | Dependent Cross-Linking | | | | |
| | NO | 0.6 | 1.9 | 5.6 | 16.7 | 50 | 0.6 | 1.9 | 5.6 | 16.7 | 50 |
| CD80 Mutation(s) | (IgV) | nM | nM | nM | nM | nM | nM | nM | nM | nM | nM |
| E35D/M47I | 177 | 1777 | 2133 | 3651 | 5792 | 7144 | 2832 | 3604 | 4702 | 5321 | 5704 |
| A71D/L72V/E95K | 192 | 1821 | 2588 | 4127 | 5553 | 7109 | 1060 | 1537 | 2517 | 3642 | 4031 |
| E35D | 198 | 1402 | 1328 | 1300 | 1318 | 1203 | 920 | 1113 | 1397 | 1765 | 2270 |
| E35D/M47I/L70M | 199 | 1609 | 2520 | 4231 | 5370 | 5780 | 2238 | 2689 | 3654 | 3907 | 3870 |
| E35D/M43L/L70M | 201 | 1349 | 1336 | 1404 | 1345 | 1573 | 1022 | 1250 | 1616 | 2046 | 2780 |
| E35D/D46V/L85Q | 203 | 1880 | 2721 | 4396 | 6023 | 7015 | 1418 | 2432 | 3306 | 3645 | 4126 |
| H18Y/A26I/E35D/ | 207 | 2081 | 2808 | 4550 | 6958 | 8747 | 1156 | 1825 | 3121 | 4329 | 5215 |
| A71D/L85Q | | | | | | | | | | | |
| E35D/M47L | 208 | 2119 | 3042 | 5615 | 7736 | 8685 | 2783 | 3846 | 4726 | 5406 | 5036 |
| E23G/A26S/E35D/T62N/ | 216 | 2022 | 3300 | 5052 | 7011 | 7855 | 1153 | 1949 | 3219 | 4042 | 4138 |
| A71D/L72V/L85M | | | | | | | | | | | |
| E35G/K54E/A71D/L72P | 219 | 1337 | 1367 | 1380 | 1430 | 1510 | 689 | 732 | 735 | 701 | 805 |
| A26E/E35D/M47L/L85Q | 221 | 1350 | 1382 | 1416 | 1371 | 1327 | 1228 | 1586 | 2004 | 2504 | 2640 |
| WT CD80 IgV-Fc | 150 | 1410 | 1349 | 1309 | 1208 | 1246 | 662 | 674 | 697 | 673 | 663 |
| WT CD80 ECD-Fc | 2 | 1344 | 1270 | 1481 | 1727 | 2202 | 692 | 705 | 847 | 875 | 1519 |
| (inert) | (ECD) | | | | | | | | | | |
| Fc only Control | 1520 | 1404 | 1390 | 1390 | 1370 | 1373 | 689 | 675 | 666 | 694 | 679 |

Some of the exemplary assessed variant CD80-IgV Fc (effector) immunomodulatory proteins, including E35D, E35D/M43L/L70M, and A26E/E35D/M47L/L85Q, did not effect CD28 costimulation when crosslinked by binding to the FcR. However, the results indicated that several exemplary assessed variants with an Fc capable of binding FcR (effector) could provide CD28 costimulation in trans with FcR crosslinking. Among these, some of the exemplary assessed CD80-IgV Fc (effector) immunomodulatory proteins, such as E35D/M47F, enhanced CD28 costimulation via crosslinking of both PD-L1 and FcR. In some cases, the results indicated enhanced CD28 costimulation by crosslinking of FcR and PD-L1 was more potent than crosslinking of PD-L1 alone.

### Example 10

### Generation of Additional Variant CD80 IgV Domains

A. Additional CD8 IgV Binding Domains and Binding Assessment

Additional CD80 variants were generated and expressed as Fc fusion proteins essentially as described in Examples 2-5. The variants were tested for binding, substantially as described in Example 7, and bioactivity, substantially described in Example 9. Results from the binding and activity studies are provided in Tables E10-E13.

1. Binding Assessment

TABLE E10
Flow Binding to Jurkats (CD28) and CHO cells stably expressing CTLA4 or PD-L1

| | | CTLA4 | | CD28 | | PD-L1 | | Ratio |
|---|---|---|---|---|---|---|---|---|
| | SEQ | MFI at | Fold | MFI at | Fold | MFI at | Fold | of |
| | ID NO | 33.3 | change | 33.3 | change | 33.3 | change | PDL1: |
| CD80 mutation(s) | (IgV) | nM | to WT CD80 | nM | to WT CD80 | nM | to WT CD80 | CD28 |
| E35D/N48K/L72V | 934 | 32731 | 17.1 | 582 | 8.8 | 3031 | 43.1 | 5 |
| E35D/T41S/N48T | 935 | 30262 | 15.8 | 72.4 | 1.1 | 2191 | 31.2 | 30 |
| D46V/M47I/A71G | 936 | 28420 | 14.8 | 1325 | 20.1 | 7328 | 104.2 | 6 |
| M47I/A71G | 937 | 27768 | 14.5 | 823 | 12.5 | 5097 | 72.5 | 6 |
| E35D/M43I/M47L/L85M | 938 | 24584 | 12.8 | 265 | 4.0 | 4878 | 69.4 | 18 |
| E35D/M43I/D46E/A71G/ | 939 | 26878 | 14.0 | 200 | 3.0 | 7138 | 101.5 | 36 |
| L85M | | | | | | | | |
| H18Y/E35D/M47L/A71G/ | 940 | 24218 | 12.6 | 528 | 8.0 | 7582 | 107.9 | 14 |
| A91S | | | | | | | | |
| E35D/M47I/N48K/I61F | 941 | 25859 | 13.5 | 816 | 12.4 | 5627 | 80.0 | 7 |
| E35D/M47V/T62S/L85Q | 942 | 31230 | 16.3 | 99.4 | 1.5 | 6653 | 94.6 | 67 |
| M43I/M47L/A71G | 943 | 23292 | 12.2 | 1000 | 15.2 | 7763 | 110.4 | 8 |
| E35D/M47V | 944 | 20893 | 10.9 | 461 | 7.0 | 2935 | 41.7 | 6 |
| E35D/M47L/A71G/L85M | 945 | 16609 | 8.7 | 199 | 3.0 | 8312 | 118.2 | 42 |
| V22A/E35D/M47L/A71G | 946 | 21855 | 11.4 | 990 | 15.0 | 8168 | 116.2 | 8 |
| E35D/M47L/A71G | 947 | 20576 | 10.7 | 626 | 9.5 | 6635 | 94.4 | 11 |
| E35D/D46E/M47I | 948 | 21394 | 11.2 | 1001 | 15.2 | 3789 | 53.9 | 4 |
| Q27H/E35D/M47I | 949 | 27530 | 14.4 | 756 | 11.5 | 3424 | 48.7 | 5 |
| E35D/D46E/L85M | 950 | 30289 | 15.8 | 164 | 2.5 | 2880 | 41.0 | 18 |
| E35D/D46E/A91G | 951 | 32189 | 16.8 | 3450 | 52.3 | 2818 | 40.1 | 1 |
| E35D/D46E | 952 | 27921 | 14.6 | 779 | 11.8 | 3757 | 53.4 | 5 |
| E35D/L97R | 953 | 22803 | 11.9 | 44.6 | 0.7 | 2614 | 37.2 | 59 |
| H18Y/E35D | 954 | 26258 | 13.7 | 479 | 7.3 | 3526 | 50.2 | 7 |
| Q27L/E35D/M47V/I61V/ | 955 | 27881 | 14.6 | 230 | 3.5 | 2705 | 38.5 | 12 |
| L85M | | | | | | | | |
| E35D/M47V/I61V/L85M | 956 | 28848 | 15.1 | 274 | 4.2 | 3054 | 43.4 | 11 |
| E35D/M47V/L85M/R94Q | 957 | 23334 | 12.2 | 23.7 | 0.4 | 3039 | 43.2 | 128 |
| E35D/M47V/N48K/L85M | 958 | 11792 | 11.5 | 413 | 10.0 | 5660 | 67.9 | 14 |
| H18Y/E35D/M47V/N48K | 959 | 11747 | 11.4 | 841 | 20.4 | 6462 | 77.5 | 8 |
| WT CD80 ECD-Fc H22.6 | 2 | 31563 | 16.5 | 43 | 0.7 | 46.3 | 0.7 | 1 |
| CD80 WT IgV-Fc | 150 | 1916 | 1.0 | 66 | 1.0 | 70.3 | 1.0 | 1 |
| Inert Fc | 1520 | 65.7 | 0.0 | 23 | 0.4 | 41 | 0.6 | 2 |

TABLE E11
Flow Binding to Jurkats (CD28) and CHO cells stably expressing CTLA4 or PD-Ll

| | | CTLA4 | | CD28 | | PD-L1 | | Ratio |
|---|---|---|---|---|---|---|---|---|
| | SEQ | MFI at | Fold | MFI at | Fold | MFI at | Fold | of |
| | ID NO | 33.3 | change | 33.3 | change | 33.3 | change | PDL1: |
| CD80 mutation(s) | (IgV) | nM | to WT CD80 | nM | to WT CD80 | nM | to WT CD80 | CD28 |
| E24D/E35D/M47L/V68M/ | 980 | 15505 | 8.8 | 15 | 0.5 | 18649 | 362.1 | 1268.6 |
| E95V/L97Q | | | | | | | | |
| E35D/D46E/M47I/T62A/ | 981 | 16987 | 9.7 | 486 | 15.5 | 18734 | 363.8 | 38.5 |
| V68M/L85M/Y87C | | | | | | | | |
| E35D/D46E/M47I/V68M/ | 982 | 14036 | 8.0 | 353 | 11.2 | 16341 | 317.3 | 46.3 |
| L85M | | | | | | | | |
| E35D/D46E/M47L/V68M/ | 983 | 15098 | 8.6 | 425 | 13.5 | 24297 | 471.8 | 57.2 |
| A71G/Y87C/K93R | | | | | | | | |
| E35D/D46E/M47L/V68M/ | 984 | 15049 | 8.6 | 403 | 12.8 | 8641 | 167.8 | 21.4 |
| T79M/L85M | | | | | | | | |
| E35D/D46E/M47L/V68M/ | 985 | 96 | 0.1 | 14 | 0.5 | 4617 | 89.7 | 325.1 |
| T79M/L85M/L97Q | | | | | | | | |
| E35D/D46E/M47V/V68M/ | 986 | 15533 | 8.9 | 1740 | 55.4 | 1723 | 33.5 | 1.0 |
| L85Q | | | | | | | | |
| E35D/M43I/M47L/V68M | 987 | 16243 | 9.3 | 1517 | 48.3 | 16912 | 328.4 | 11.1 |
| E35D/M47I/V68M/Y87N | 988 | 17860 | 10.2 | 3553 | 113.2 | 13145 | 255.2 | 3.7 |
| E35D/M47L/V68M/E95V/ | 989 | 14955 | 8.5 | 14 | 0.5 | 18600 | 361.2 | 1300.7 |
| L97Q | | | | | | | | |
| E35D/M47L/Y53F/V68M/ | 990 | 16013 | 9.1 | 383 | 12.2 | 25024 | 485.9 | 65.3 |
| A71G/K93R/E95V | | | | | | | | |
| E35D/M47V/N48KN68M/ | 991 | 16604 | 9.5 | 302 | 9.6 | 22770 | 442.1 | 75.4 |
| A71G/L85M | | | | | | | | |
| E35D/M47V/N48KN68M/ | 992 | 15581 | 8.9 | 245 | 7.8 | 7618 | 147.9 | 31.1 |
| L85M | | | | | | | | |
| E35D/M47V/V68M/L85M | 993 | 15997 | 9.1 | 201 | 6.4 | 9177 | 178.2 | 45.7 |
| E35D/M47V/V68M/L85M/ | 994 | 13936 | 7.9 | 509 | 16.2 | 1721 | 33.4 | 3.4 |
| Y87D | | | | | | | | |
| E35D/T41S/D46E/M47I/ | 995 | 18369 | 10.5 | 476 | 15.2 | 14790 | 287.2 | 31.1 |
| V68M/K93R/E95V | | | | | | | | |
| H18Y/E35D/D46E/M47I/ | 996 | 23300 | 13.3 | 244 | 7.8 | 18806 | 365.2 | 77.1 |
| V68M/R94L | | | | | | | | |
| H18Y/E35D/M38I/M47L/ | 997 | 139 | 0.1 | 16.7 | 0.5 | 3589 | 69.7 | 214.9 |
| V68M/L85M | | | | | | | | |
| H18Y/E35D/M47I/V68M/ | 998 | 18626 | 10.6 | 4038 | 128.6 | 14988 | 291.0 | 3.7 |
| Y87N | | | | | | | | |
| H18Y/E35D/M47L/V68M/ | 999 | 19541 | 11.1 | 437 | 13.9 | 18669 | 362.5 | 42.7 |
| A71G/L85M | | | | | | | | |
| H18Y/E35D/M47L/V68M/ | 1000 | 20475 | 11.7 | 14.5 | 0.5 | 14750 | 286.4 | 1017.2 |
| E95V/L97Q | | | | | | | | |
| H18Y/E35D/M47L/Y53F/ | 1001 | 146 | 0.1 | 15.7 | 0.5 | 5105 | 99.1 | 325.2 |
| V68M/A71G | | | | | | | | |
| H18Y/E35D/M47L/Y53F/ | 1002 | 18356 | 10.5 | 334 | 10.6 | 23390 | 454.2 | 70.0 |
| V68M/A71G/K93R/E95V | | | | | | | | |
| H18Y/E35D/M47V/V68M/ | 1003 | 18367 | 10.5 | 373 | 11.9 | 16774 | 325.7 | 45.0 |
| L85M | | | | | | | | |
| H18Y/E35D/V68M/A71G/ | 1004 | 18281 | 10.4 | 16 | 0.5 | 14990 | 291.1 | 954.8 |
| R94Q/E95V | | | | | | | | |
| H18Y/E35D/V68M/L85M/ | 1005 | 19766 | 11.3 | 14 | 0.4 | 14410 | 279.8 | 1036.7 |
| R94Q | | | | | | | | |
| H18Y/E35D/V68M/T79M/ | 1006 | 16287 | 9.3 | 1041 | 33.2 | 14907 | 289.5 | 14.3 |
| L85M | | | | | | | | |
| H18Y/V22D/E35D/M47V/ | 1007 | 15798 | 9.0 | 257 | 8.2 | 12867 | 249.8 | 50.1 |
| N48K/V68M | | | | | | | | |
| Q27L/Q33L/E35D/T41S/ | 1008 | 178 | 0.1 | 15 | 0.5 | 16492 | 320.2 | 1129.6 |
| M47V/N48K/V68M/L85M | | | | | | | | |
| Q33L/E35D/M47V/T62S/ | 1009 | 86 | 0.0 | 15 | 0.5 | 16838 | 327.0 | 1107.8 |
| V68M/L85M | | | | | | | | |
| Q33R/E35D/M38I/M47L/ | 1010 | 107 | 0.1 | 15 | 0.5 | 16502 | 320.4 | 1107.5 |
| V68M | | | | | | | | |
| R29C/E35D/M47L/V68M/ | 1011 | 91 | 0.1 | 16 | 0.5 | 16251 | 315.6 | 997.0 |
| A71G/L85M | | | | | | | | |
| S21P/E35D/K37E/D46E/ | 1012 | 20616 | 11.8 | 540 | 17.2 | 17833 | 346.3 | 33.0 |
| M47I/V68M | | | | | | | | |
| S21P/E35D/K37E/D46E/ | 1013 | 20142 | 11.5 | 284 | 9.0 | 17789 | 345.4 | 62.6 |
| M47I/V68M/R94L | 1014 | 21255 | 12.1 | 15.6 | 0.5 | 19969 | 387.7 | 1280.1 |
| T13R/E35D/M47LN68M | | | | | | | | |
| T13R/Q27L/Q33L/E35D/ | 1016 | 109 | 0.1 | 14.6 | 0.5 | 3272 | 63.5 | 224.1 |
| T41S/M47V/N48K/V68M/ | | | | | | | | |
| L85M | | | | | | | | |
| T13R/Q33L/E35D/M47L/ | 1017 | 141 | 0.1 | 15.7 | 0.5 | 3228 | 62.7 | 205.6 |
| V68M/L85M | | | | | | | | |
| T13R/Q33L/E35D/M47V/ | 1018 | 105 | 0.1 | 16 | 0.5 | 3968 | 77.0 | 248.0 |
| T62S/V68M/L85M | | | | | | | | |
| T13R/Q33R/E35D/M38I/ | 1019 | 193 | 0.1 | 13.8 | 0.4 | 4482 | 87.0 | 324.8 |
| M47L/V68M | | | | | | | | |
| T13R/Q33R/E35D/M38I/ | 1020 | 20652 | 11.8 | 1111 | 35.4 | 19157 | 372.0 | 17.2 |
| M47L/V68M/E95V/L97Q | | | | | | | | |
| T13R/Q33R/E35D/M38I/ | 1021 | 22011 | 12.6 | 14.2 | 0.5 | 1106 | 21.5 | 77.9 |
| M47L/V68M/L85M | | | | | | | | |
| T13R/Q33R/E35D/M38I/ | 1022 | 19105 | 10.9 | 15.2 | 0.5 | 20366 | 395.5 | 1339.9 |
| M47L/V68M/L85M/R94Q | | | | | | | | |
| T13R/Q33R/E35D/M47L/ | 1023 | 20738 | 11.8 | 14.1 | 0.4 | 14680 | 285.0 | 1041.1 |
| V68M | | | | | | | | |
| T13R/Q33R/E35D/M47L/ | 1024 | 13438 | 7.7 | 112 | 3.6 | 18938 | 367.7 | 169.1 |
| V68M/L85M | | | | | | | | |
| V22D/E24D/E35D/M47L/ | 1025 | 19403 | 11.1 | 1254 | 39.9 | 15418 | 299.4 | 12.3 |
| V68M | | | | | | | | |
| V22D/E24D/E35D/M47L/ | 1026 | 14574 | 8.3 | 1183 | 37.7 | 19047 | 369.8 | 16.1 |
| V68M/L85M/D90G | | | | | | | | |
| V22D/E24D/E35D/M47V/ | 1027 | 16899 | 9.6 | 191 | 6.1 | 17793 | 345.5 | 93.2 |
| V68M | | | | | | | | |
| WT CD80 ECD-Fc | 2 | 1753 | 1.0 | 31 | 1.0 | 52 | 1.0 | 1.6 |
| CD80 WT IgV-Fc | 150 | 26392 | 15.1 | 95 | 3.0 | 44 | 0.9 | 0.5 |

Bioactivity Assessment

TABLE E12
Jurkat/IL2 + CHO/OKT3/PD-L1 Reporter Assay:
Relative Luciferase Units (RLU)

| | SEQ | CD80 | Fold Increase |
|---|---|---|---|
| | ID | Cone | over WT |
| CD80 Mutation(s) | NO: | 5.0 nM | CD80-IgV-Fe |
| E35D/N48K/L72V | 934 | 1731 | 4.3 |
| E35D/T41S/N48T | 935 | 1136 | 2.8 |
| D46V/M47I/A71G | 936 | 1601 | 4.0 |
| M47I/A71G | 937 | 1762 | 4.4 |
| E35D/M43I/M47L/L85M | 938 | 1427 | 3.6 |
| E35D/M43I/D46E/A71G/ | 939 | 1475 | 3.7 |
| L85M | | | |
| H18Y/E35D/M47L/ | 940 | 1898 | 4.7 |
| A71G/A91S | | | |
| E35D/M47I/N48K/I61F | 941 | 2078 | 5.2 |
| E35D/M47V/T62S/L85Q | 942 | 1402 | 3.5 |
| M43I/M47L/A71G | 943 | 1641 | 4.1 |
| E35D/M47V | 944 | 1353 | 3.4 |
| E35D/M47L/A71G/L85M | 945 | 1513 | 3.8 |
| V22A/E35D/M47L/A71G | 946 | 2583 | 6.5 |
| E35D/M47L/A71G | 947 | 1954 | 4.9 |
| E35D/D46E/M47I | 948 | 1915 | 4.8 |
| Q27H/E35D/M47I | 949 | 1829 | 4.6 |
| E35D/D46E/L85M | 950 | 1413 | 3.5 |
| E35D/D46E/A91G | 951 | 395 | 1.0 |
| E35D/D46E | 952 | 1961 | 4.9 |
| E35D/L97R | 953 | 914 | 2.3 |
| H18Y/E35D | 954 | 1990 | 5.0 |
| Q27L/E35D/M47V/ | 955 | 1166 | 2.9 |
| I61V/L85M | | | |
| E35D/M47V/I61V/L85M | 956 | 1176 | 2.9 |
| E35D/M47V/L85M/R94Q | 957 | 466 | 1.2 |
| E35D/M47V/N48K/L85M | 958 | 2116 | 5.3 |
| H18Y/E35D/M47V/N48K | 959 | 2146 | 5.4 |
| CD80 WT IgV-Fc | 150 | 400 | 1.0 |
| CD80 ECD-Fc | 2 | 521 | 1.3 |

TABLE E13
Jurkat/IL2 + CHO/OKT3/PD-L1 Reporter Assay:
Relative Luciferase Units (RLU)

| | SEQ ID | CD80 | Fold Increase |
|---|---|---|---|
| | NO | Cone | over WT CD80- |
| CD80 Mutation(s) | (IgV) | 5.0 nM | IgV-Fe |
| E24D/E35D/M47L/V68M/E95V/L97Q | 980 | 1087 | 2.7 |
| E35D/D46E/M47I/T62A/V68M/L85M/Y87C | 981 | 1104 | 2.8 |
| E35D/D46E/M47I/V68M/L85M | 982 | 1230 | 3.1 |
| E35D/D46E/M47L/V68M/A71G/Y87C/K93R | 983 | 1198 | 3.0 |
| E35D/D46E/M47L/V68M/T79M/L85M | 984 | 1137 | 2.8 |
| E35D/D46E/M47L/V68M/T79M/L85M/L97Q | 985 | 160 | 0.4 |
| E35D/D46E/M47V/V68M/L85Q | 986 | 1006 | 2.5 |
| E35D/M43I/M47L/V68M | 987 | 1072 | 2.7 |
| E35D/M47I/V68M/Y87N | 988 | 958 | 2.4 |
| E35D/M47L/V68M/E95V/L97Q | 989 | 1086 | 2.7 |
| E35D/M47L/Y53F/V68M/A71G/K93R/E95V | 990 | 1546 | 3.9 |
| E35D/M47V/N48K/V68M/A71G/L85M | 991 | 1422 | 3.6 |
| E35D/M47V/N48K/V68M/L85M | 992 | 1203 | 3.0 |
| E35D/M47V/V68M/L85M | 993 | 1167 | 2.9 |
| E35D/M47V/V68M/L85M/Y87D | 994 | 1181 | 3.0 |
| E35D/T41S/D46E/M47I/V68M/K93R/E95V | 995 | 1165 | 2.9 |
| H18Y/E35D/D46E/M47I/V68M/R94L | 996 | 1425 | 3.6 |
| H18Y/E35D/M38I/M47L/V68M/L85M | 997 | 198 | 0.5 |
| H18Y/E35D/M47I/V68M/Y87N | 998 | 1117 | 2.8 |
| H18Y/E35D/M47L/V68M/A71G/L85M | 999 | 1219 | 3.0 |
| H18Y/E35D/M47L/V68M/E95V/L97Q | 1000 | 225 | 0.6 |
| H18Y/E35D/M47L/Y53F/V68M/A71G | 1001 | 120 | 0.3 |
| H18Y/E35D/M47L/Y53F/V68M/A71G/ | 1002 | 1190 | 3.0 |
| K93R/E95V | | | |
| H18Y/E35D/M47V/V68M/L85M | 1003 | 1013 | 2.5 |
| H18Y/E35D/V68M/A71G/R94Q/E95V | 1004 | 183 | 0.5 |
| H18Y/E35D/V68M/L85M/R94Q | 1005 | 195 | 0.5 |
| H18Y/E35D/V68M/T79M/L85M | 1006 | 1161 | 2.9 |
| H18Y/V22D/E35D/M47V/N48K/V68M | 1007 | 1072 | 2.7 |
| Q27L/Q33L/E35D/T41S/M47V/N48K/ | 1008 | 170 | 0.4 |
| V68M/L85M | | | |
| Q33L/E35D/M47V/T62S/V68M/L85M | 1009 | 158 | 0.4 |
| Q33R/E35D/M38I/M47L/V68M | 1010 | 147 | 0.4 |
| R29C/E35D/M47L/V68M/A71G/L85M | 1011 | 155 | 0.4 |
| S21P/E35D/K37E/D46E/M47I/V68M | 1012 | 1064 | 2.7 |
| S21P/E35D/K37E/D46E/M47I/V68M/R94L | 1013 | 1205 | 3.0 |
| T13R/E35D/M47L/V68M | 1014 | 1021 | 2.6 |
| T13R/Q27L/Q33L/E35D/T41S/M47V/N48KN | 1016 | 170 | 0.4 |
| 68M/L85M | | | |
| T13R/Q33L/E35D/M47L/V68M/L85M | 1017 | 153 | 0.4 |
| T13R/Q33L/E35D/M47V/T62S/V68M/L85M | 1018 | 136 | 0.3 |
| T13R/Q33R/E35D/M38I/M47L/V68M | 1019 | 152 | 0.4 |
| T13R/Q33R/E35D/M38I/M47L/ | 1020 | 993 | 2.5 |
| V68M/E95V/L97Q | | | |
| T13R/Q33R/E35D/M38I/M47L/V68M/L85M | 1021 | 153 | 0.4 |
| T13R/Q33R/E35D/M38I/M47L/V68M/L85M/ | 1022 | 580 | 1.5 |
| R94Q | | | |
| T13R/Q33R/E35D/M47L/V68M | 1023 | 399 | 1.0 |
| T13R/Q33R/E35D/M47L/V68M/L85M | 1024 | 1160 | 2.9 |
| V22D/E24D/E35D/M47L/V68M | 1025 | 974 | 2.4 |
| V22D/E24D/E35D/M47L/V68M/L85M/D90G | 1026 | 963 | 2.4 |
| V22D/E24D/E35D/M47V/V68M | 1027 | 1023 | 2.6 |
| CD80 WT IgV-Fc | 150 | 400 | 1.0 |
| WT CD80 ECD-Fc H22.6 | 2 | 521 | 1.3 |

B. Generation of Variant CD8r IgV Binding Domains and High-Throughput Selection

Additional CD80 IgV variants were selected after generating 300 CD80 IgV-Fc constructs from the yeast outputs described in Example 7. Supernatants containing the CD80 IgV-Fc proteins were then screened for PD-L1 binding in a 96-well plate format using an Octet® System. Variants that exhibited high PD-L1 binding were selected and rescreened for binding as described in Example 7 above, and variants were selected that exhibited high PD-L11 binding. Exemplary variants and the FACS binding data are provided in Table E14. The selected variants also were assessed for bioactivity using the methods substantially as described in Example 9, and the results are shown in Table E15.

TABLE E14
Flow Binding to Jurkats (CD28) and CHO cells stably expressing CTLA4 or PD-L1

| | | CTLA4 | | CD28 | | PD-L1 | | Ratio |
|---|---|---|---|---|---|---|---|---|
| | SEQ | MFI at | Fold | MFI at | Fold | MFI at | Fold | of |
| | ID NO | 33.3 | change | 33.3 | change | 33.3 | change | PDL1: |
| CD80 mutation(s) | (IgV) | nM | to WT CD80 | nM | to WT | nM | to WT | CD28 |
| A26E/Q27R/E35D/M47L/ | 538 | 10848 | 10.6 | 78 | 1.9 | 9315 | 111.7 | 119 |
| N48Y/L85Q | | | | | | | | |
| E35D/D46E/M47L/V68M/ | 539 | 214 | 0.2 | 15 | 0.4 | 13200 | 158.3 | 863 |
| L85Q/F92L | | | | | | | | |
| E35D/M47I/T62S/L85Q/ | 540 | 8913 | 8.7 | 111 | 2.7 | 8417 | 100.9 | 76 |
| E88D | | | | | | | | |
| E24D/Q27R/E35D/T41S | 541 | 13867 | 13.5 | 66 | 1.6 | 2858 | 34.3 | 44 |
| M47V/L85Q | | | | | | | | |
| S15T/H18Y/E35D/M47V/ | 542 | 10994 | 10.7 | 1068 | 25.9 | 13883 | 166.5 | 13 |
| T62A/N64S/A71G/L85Q/ | | | | | | | | |
| D90N | | | | | | | | |
| E35D/M47LN68M/A71G/ | 543 | 10332 | 10.1 | 1400 | 33.9 | 16832 | 201.8 | 12 |
| L85Q/D90G | | | | | | | | |
| H18Y/E35D/M47I/V68M/ | 544 | 10036 | 9.8 | 1905 | 46.1 | 14487 | 173.7 | 8 |
| A71G/R94L | | | | | | | | |
| deltaE10-A98 | 545 | 125 | 0.1 | 15 | 0.4 | 45 | 0.5 | 3 |
| Q33R/M47V/T62N/A71G | 546 | 308 | 0.3 | 17 | 0.4 | 12216 | 146.5 | 719 |
| H18Y/V22A/E35D/T41S/ | 547 | 10290 | 10.0 | 1591 | 38.5 | 8459 | 101.4 | 5 |
| M47V/T62N/A71G/A91G | | | | | | | | |
| CD80 WT IgV-Fc | 150 | 1026 | 1.0 | 41 | 1.0 | 83 | 1.0 | 2 |
| CD80 ECD-Fc | 2 | 31725 | 30.9 | 30 | 0.7 | 68 | 0.8 | 2 |

TABLE E15
Jurkat/IL2 + CHO/OKT3/PD-L1 Reporter Assay:
Relative Luciferase Units (RLU)

| | SEQ | CD80-Fe | Fold Increase |
|---|---|---|---|
| | ID NO | Cone | over WT CD80- |
| CD80 Mutations | (IgV) | 5.0 nM | IgV-Fe |
| A26E/Q27R/E35D/M47L/N48Y/L85Q | 538 | 433 | 1.1 |
| E35D/D46E/M47L/V68M/L85Q/F92L | 539 | 2551 | 6.4 |
| E35D/M47I/T62S/L85Q/E88D | 540 | 605 | 1.5 |
| E24D/Q27R/E35D/T41S/M47V/L85Q | 541 | 147 | 0.4 |
| S15T/H18Y/E35D/M47V/T62A/ | 542 | 872 | 2.2 |
| N64S/A71G/L85Q/D90N | | | |
| E35D/M47L/V68M/A71G/L85Q/D90G | 543 | 936 | 2.3 |
| H18Y/E35D/M47I/V68M/A71G/R94L | 544 | 879 | 2.2 |
| deltaE10-A98 | 545 | 137 | 0.3 |
| Q33R/M47V/T62N/A71G | 546 | 149 | 0.4 |
| H18Y/V22A/E35D/T41S/M47V/T62N/A71G/A91G | 547 | 1045 | 2.6 |
| CD80 WT IgV-Fc | 150 | 400 | 1.0 |
| CD80 ECD-Fc | 2 | 521 | 1.3 |

C. Generation of CD80 IgV Consensus Variants

Consensus variants of CD80 IgV variants were designed based on the alignments of outputs from all of the yeast selections described above. The consensus sequences were then used to generate CD80 IgV-Fc proteins that were then tested for binding and bioactivity as described above. The binding and bioactivity results are provided in Tables E16 and E17, respectively.

TABLE E16
Flow Binding to Jurkats (CD28) and CHO cells stably expressing
CTLA4 or PD-L1

| | | CTLA4 | | CD28 | | PD-L1 | | |
|---|---|---|---|---|---|---|---|---|
| | | | Fold | | Fold | | Fold | |
| | | MFI | change | MFI | change | MFI | change | Ratio |
| | | at | to | at | to | at | to | of |
| | SEQ ID | 33.3 | WT | 33.3 | WT | 33.3 | WT | PDL1: |
| CD80 Mutations | NO (IgV) | nM | CD80 | nM | CD80 | nM | CD80 | CD28 |
| H18Y/E35D/D46E/M47I/ | 996 | 19236 | 18.4 | 1006 | 24.4 | 2082 | 29.4 | 2.1 |
| V68M/R94L | | | | | | | | |
| H18Y/E35D/M47I/V68M/ | 998 | 19722 | 18.9 | 1429 | 34.7 | 9299 | 131.2 | 6.5 |
| Y87N | | | | | | | | |
| H18Y/E35D/M47L/V68M/ | 999 | 20660 | 19.8 | 2848 | 69.1 | 9894 | 139.5 | 3.5 |
| A71G/L85M | | | | | | | | |
| H18Y/E35D/M47L/V68M/ | 1000 | 18022 | 17.2 | 2602 | 63.2 | 9629 | 135.8 | 3.7 |
| E95V/L97Q | | | | | | | | |
| H18Y/E35D/M47L/Y53F/ | 1001 | 19528 | 18.7 | 478 | 11.6 | 9576 | 135.1 | 20.0 |
| V68M/A71G | | | | | | | | |
| H18Y/E35D/M47L/Y53F/ | 1002 | 19754 | 18.9 | 2194 | 53.3 | 9339 | 131.7 | 4.3 |
| V68M/A71G/K93R/E95V | | | | | | | | |
| H18Y/E35D/M47V/V68M/ | 1003 | 19306 | 18.5 | 1387 | 33.7 | 3094 | 43.6 | 2.2 |
| L85M | | | | | | | | |
| H18Y/E35D/V68M/A71G/ | 1004 | 19396 | 18.6 | 455 | 11.0 | 1836 | 25.9 | 4.0 |
| R94Q/E95V | | | | | | | | |
| H18Y/E35D/V68M/L85M/ | 1005 | 21955 | 21.0 | 962 | 23.3 | 9283 | 130.9 | 9.6 |
| R94Q | | | | | | | | |
| CD80 WT IgV-Fc | 150 | 1045 | 1.0 | 41.2 | 1.0 | 70.9 | 1.0 | 1.7 |
| CD80 ECD-Fc | 2 | 46137 | 44.2 | 46 | 1.1 | 58 | 0.8 | 1.3 |

TABLE E17
Jurkat/IL2 + CHO/OKT3/PD-L1 Reporter Assay:
Relative Luciferase Units (RLU)

| | SEQ | CD80 | Fold Increase |
|---|---|---|---|
| | ID | Cone | over WT |
| CD80 Mutations | NO: | 5.0 nM | CD80-IgV-Fe |
| H18Y/E35D/D46E/M47I/V68M/ | 996 | 2850 | 7.1 |
| R94L | | | |
| H18Y/E35D/M47I/V68M/Y87N | 998 | 2196 | 5.5 |
| H18Y/E35D/M47L/V68M/A71G/ | 999 | 2193 | 5.5 |
| L85M | | | |
| H18Y/E35D/M47L/V68M/E95V/L97 [symbol] | 1000 | 2052 | 5.1 |
| H18Y/E35D/M47L/Y53F/V68M/ | 1001 | 2277 | 5.7 |
| A71G | | | |
| H18Y/E35D/M47L/Y53F/V68M/ | 1002 | 2212 | 5.5 |
| A71G/K93R/E95V | | | |
| H18Y/E35D/M47V/V68M/L85M | 1003 | 2575 | 6.4 |
| H18Y/E35D/V68M/A71G/R94Q/ | 1004 | 1968 | 4.9 |
| E95V | | | |
| H18Y/E35D/V68M/L85M/R94Q | 1005 | 2215 | 5.5 |
| CD80 WT IgV-Fc | 150 | 400 | 1.0 |
| CD80 ECD-Fc | 2 | 521 | 1.3 |

[symbol]

indicates data missing or illegible when filed

### Example 11

### Assessment of Binding Activity of a Panel of CD80 IgV Variants

To identify residues involved in binding and activity with reference to a selected set of variants set forth in SEQ ID NOs: 465, 491, and 495, a panel of reversion (back) mutations were designed and expressed as Fc fusion proteins substantially as described in Examples 4 and 5. The variants generated contained between 1 and 6 mutations found in SEQ ID NOS: 465, 491, and 495 in various combinations as set forth in Table E18.

TABLE E18
Additional CD80 Variants

| Mutation | SEQ ID NO: |
|---|---|
| E35D | 198 |
| D46V | 1028 |
| M47L | 1029 |
| V68M | 1030 |
| L85Q | 1031 |
| E35D/D46V | 1032 |
| E35D/M47L | 208 |
| E35D/L85Q | 1034 |
| D46V/M47L | 1035 |
| D46V/V68M | 1036 |
| D46V/L85Q | 1037 |
| M47L/V68M | 1038 |
| M47L/L85Q | 1039 |
| V68M/L85Q | 1040 |
| E35D/D46V/M47L | 1041 |
| E35D/D46V/V68M | 1042 |
| E35D/D46V/L85Q | 1043 |
| E35D/M47L/V68M | 971 |
| E35D/M47L/L85Q | 418 |
| E35D/V68M/L85Q | 1044 |
| D46V/M47L/V68M | 1045 |
| D46V/M47L/L85Q | 1046 |
| D46V/V68M/L85Q | 1047 |
| M47L/V68M/L85Q | 1048 |
| E35D/D46V/M47L/V68M | 976 |
| E35D/D46V/M47L/L85Q | 1049 |
| E35D/D46V/V68M/L85Q | 1050 |
| E35D/M47L/V68M/L85Q | 1051 |
| D46V/M47L/V68M/L85Q | 1052 |
| E35D/D46V/M47L/V68M/L85Q | 975 |
| M47V | 1053 |
| N48K | 1054 |
| K89N | 1055 |
| E35D/M47V | 944 |
| E35D/N48K | 1056 |
| E35D/K89N | 1057 |
| M47V/N48K | 1058 |
| M47V/V68M | 1059 |
| M47V/K89N | 1060 |
| N48K/V68M | 1061 |
| N48K/K89N | 1062 |
| V68M/K89N | 1063 |
| E35D/M47V/N48K | 1064 |
| E35D/M47V/V68M | 1065 |
| E35D/M47V/K89N | 1066 |
| E35D/N48K/V68M | 1067 |
| E35D/N48K/K89N | 1068 |
| E35D/V68M/K89N | 1069 |
| M47V/N48K/V68M | 1070 |
| M47V/N48K/K89N | 1071 |
| M47V/V68M/K89N | 1072 |
| N48K/V68M/K89N | 1073 |
| E35D/M47V/N48K/V68M | 979 |
| E35D/M47V/N48K/K89N | 1074 |
| E35D/M47V/V68M/K89N | 1075 |
| E35D/N48K/V68M/K89N | 1076 |
| M47V/N48K/V68M/K89N | 1077 |
| E35D/D46V/M47V/N48K/V68M | 1078 |
| E35D/D46V/M47V/V68M/L85Q | 1079 |
| E35D/D46V/M47V/V68M/K89N | 1080 |
| E35D/M47V/N48K/V68M/L85Q | 1081 |
| E35D/M47V/N48K/V68M/K89N | 465 |
| E35D/M47V/V68M/L85Q/K89N | 1082 |
| A26E/E35D/M47L/V68M/A71G/D90G | 1083 |
| H18Y/E35D/M47L/V68M/A71G/D90G | 1084 |
| H18Y/A26E/M47L/V68M/A71G/D90G | 1085 |
| H18Y/A26E/E35D/V68M/A71G/D90G | 1086 |
| H18Y/A26E/E35D/M47L/A71G/D90G | 1087 |
| H18Y/A26E/E35D/M47L/V68M/D90G | 1088 |
| H18Y/A26E/E35D/M47L/V68M/A71G | 1089 |
| E35D/M47L/V68M/A71G/D90G | 1090 |
| H18Y/M47L/V68M/A71G/D90G | 1091 |
| H18Y/A26E/V68M/A71G/D90G | 1092 |
| H18Y/A26E/E35D/A71G/D90G | 1093 |
| H18Y/A26E/E35D/M47L/D90G | 1094 |
| H18Y/A26E/E35D/M47L/V68M | 1095 |
| A26E/M47L/V68M/A71G/D90G | 1096 |
| A26E/E35D/V68M/A71G/D90G | 1097 |
| A26E/E35D/M47L/A71G/D90G | 1098 |
| A26E/E35D/M47L/V68M/D90G | 1099 |
| A26E/E35D/M47L/V68M/A71G | 1100 |
| H18Y/E35D/V68M/A71G/D90G | 1101 |
| H18Y/E35D/M47L/A71G/D90G | 1102 |
| H18Y/E35D/M47L/V68M/D90G | 1103 |
| H18Y/E35D/M47L/V68M/A71G | 1104 |
| H18Y/A26E/M47L/A71G/D90G | 1105 |
| H18Y/A26E/M47L/V68M/D90G | 1106 |
| H18Y/A26E/M47L/V68M/A71G | 1107 |
| H18Y/A26E/E35D/V68M/D90G | 1108 |
| H18Y/A26E/E35D/V68M/A71G | 1109 |
| H18Y/A26E/E35D/M47L/A71G | 1110 |
| M47L/V68M/A71G/D90G | 1111 |
| H18Y/V68M/A71G/D90G | 1112 |
| H18Y/A26E/A71G/D90G | 1113 |
| H18Y/A26E/E35D/D90G | 1114 |
| H18Y/A26E/E35D/M47L | 1115 |
| E35D/V68M/A71G/D90G | 1116 |
| E35D/M47L/A71G/D90G | 1117 |
| E35D/M47L/V68M/D90G | 1118 |
| E35D/M47L/V68M/A71G | 1119 |
| A26E/V68M/A71G/D90G | 1120 |
| A26E/M47L/A71G/D90G | 1121 |
| A26E/M47L/V68M/D90G | 1122 |
| A26E/M47L/V68M/A71G | 1123 |
| A26E/E35D/V68M/D90G | 1125 |
| A26E/E35D/V68M/A71G | 1126 |
| A26E/E35D/M47L/D90G | 1127 |
| A26E/E35D/M47L/A71G | 1128 |
| H18Y/M47L/A71G/D90G | 1129 |
| H18Y/M47L/V68M/D90G | 1130 |
| H18Y/M47L/V68M/A71G | 1131 |
| H18Y/E35D/A71G/D90G | 1132 |
| H18Y/E35D/M47L/A71G | 1136 |
| H18Y/A26E/V68M/D90G | 1138 |
| H18Y/A26E/V68M/A71G | 1139 |
| H18Y/A26E/M47L/D90G | 1140 |
| H18Y/A26E/M47L/A71G | 1141 |
| H18Y/A26E/E35D/V68M | 1144 |

The variants were tested for binding and bioactivity as described above. The binding results are set forth in Tables E19 and E20, and the bioactivity results are set forth in Tables E21 and E22.

TABLE E19
Flow Binding to Jurkats (CD28) and CHO cells stably expressing CTLA4 or PD-
L1

| | | CTLA4 | | CD28 | | PD-L1 | | |
|---|---|---|---|---|---|---|---|---|
| | | | Fold | | Fold | | Fold | |
| | | MFI | change | MFI | change | MFI | change | Ratio |
| | | at | to | at | to | at | to | of |
| | SEQ ID | 33.3 | WT | 33.3 | WT | 33.3 | WT | PDL1: |
| Mutation(s) | NO (IgV) | nM | CD80 | nM | CD80 | nM | CD80 | CD28 |
| E35D | 198 | 42923 | 1.1 | 134 | 0.2 | 2584 | 20.2 | 19.3 |
| M47L | 2814 | 30774 | 0.8 | 309 | 0.4 | 1895 | 14.8 | 6.1 |
| V68M | 2815 | 568 | 0.0 | 37.9 | 0.1 | 118 | 0.9 | 3.1 |
| L85Q | 2816 | 3002 | 0.1 | 35 | 0.0 | 97 | 0.8 | 2.8 |
| E35D/D46V | 2817 | 50112 | 1.2 | 880 | 1.2 | 3971 | 31.0 | 4.5 |
| E35D/M47L | 208 | 48010 | 1.2 | 411 | 0.6 | 7529 | 58.8 | 18.3 |
| D46V/M47L | 2820 | 49711 | 1.2 | 918 | 1.3 | 3905 | 30.5 | 4.3 |
| D46V/V68M | 2821 | 5334 | 0.1 | 556 | 0.8 | 2271 | 17.7 | 4.1 |
| D46V/L85Q | 2822 | 41896 | 1.0 | 131 | 0.2 | 2197 | 17.2 | 16.8 |
| M47L/L85Q | 2824 | 31671 | 0.8 | 88.1 | 0.1 | 5801 | 45.3 | 65.8 |
| V68M/L85Q | 2825 | 3288 | 0.1 | 91.7 | 0.1 | 347 | 2.7 | 3.8 |
| E35D/D46V/M47L | 2826 | 44977 | 1.1 | 1165 | 1.6 | 7988 | 62.4 | 6.9 |
| E35D/D46V/V68M | 2827 | 31195 | 0.8 | 1820 | 2.6 | 26114 | 204.0 | 14.3 |
| E35D/D46V/L85Q | 2828 | 48005 | 1.2 | 196 | 0.3 | 4039 | 31.6 | 20.6 |
| E35D/M47L/V68M | 2756 | 28603 | 0.7 | 1243 | 1.8 | 27896 | 217.9 | 22.4 |
| E35D/M47L/L85Q | 2203 | 12909 | 0.3 | 46.3 | 0.1 | 6097 | 47.6 | 131.7 |
| E35D/V68M/L85Q | 2829 | 42761 | 1.1 | 76.2 | 0.1 | 5971 | 46.6 | 78.4 |
| D46V/M47L/V68M | 2830 | 34688 | 0.9 | 2183 | 3.1 | 28020 | 218.9 | 12.8 |
| D46V/M47L/L85Q | 2831 | 40153 | 1.0 | 567 | 0.8 | 5976 | 46.7 | 10.5 |
| D46V/V68M/L85Q | 2832 | 7567 | 0.2 | 104 | 0.1 | 4170 | 32.6 | 40.1 |
| M47L/V68M/L85Q | 2833 | 11134 | 0.3 | 60.9 | 0.1 | 4039 | 31.6 | 66.3 |
| E35D/D46V/M47L/V68M | 2761 | 34319 | 0.8 | 1808 | 2.6 | 29266 | 228.6 | 16.2 |
| E35D/D46V/M47L/L85Q | 2834 | 38150 | 0.9 | 268 | 0.4 | 7523 | 58.8 | 28.1 |
| E35D/D46V/V68M/L85Q | 2835 | 32176 | 0.8 | 261 | 0.4 | 23637 | 184.7 | 90.6 |
| E35D/M47L/V68M/L85Q | 2836 | 28106 | 0.7 | 159 | 0.2 | 15307 | 119.6 | 96.3 |
| D46V/M47L/V68M/L85Q | 2837 | 32521 | 0.8 | 660 | 0.9 | 29743 | 232.4 | 45.1 |
| E35D/D46V/M47L/V68M/ | 2760 | 26207 | 0.6 | 464 | 0.7 | 28418 | 222.0 | 61.2 |
| L85Q | | | | | | | | |
| M47V | 2838 | 33341 | 0.8 | 68.7 | 0.1 | 2317 | 18.1 | 33.7 |
| N48K | 2839 | 4952 | 0.1 | 60.1 | 0.1 | 481 | 3.8 | 8.0 |
| K89N | 2840 | 944 | 0.0 | 56.3 | 0.1 | 52.8 | 0.4 | 0.9 |
| E35D/M47V | 2729 | 44569 | 1.1 | 501 | 0.7 | 6796 | 53.1 | 13.6 |
| E35D/N48K | 2841 | 41325 | 1.0 | 194 | 0.3 | 6545 | 51.1 | 33.7 |
| E35D/K89N | 2842 | 21755 | 0.5 | 236 | 0.3 | 757 | 5.9 | 3.2 |
| M47V/N48K | 2843 | 44640 | 1.1 | 413 | 0.6 | 3083 | 24.1 | 7.5 |
| M47V/V68M | 2844 | 7282 | 0.2 | 328 | 0.5 | 4294 | 33.5 | 13.1 |
| M47V/K89N | 2845 | 32381 | 0.8 | 197 | 0.3 | 622 | 4.9 | 3.2 |
| N48K/V68M | 2846 | 2341 | 0.1 | 118 | 0.2 | 754 | 5.9 | 6.4 |
| N48K/K89N | 2847 | 4370 | 0.1 | 170 | 0.2 | 186 | 1.5 | 1.1 |
| V68M/K89N | 2848 | 2330 | 0.1 | 210 | 0.3 | 538 | 4.2 | 2.6 |
| E35D/M47V/N48K | 2849 | 47430 | 1.2 | 771 | 1.1 | 4852 | 37.9 | 6.3 |
| E35D/M47V/V68M | 2850 | 26988 | 0.7 | 791 | 1.1 | 16645 | 130.0 | 21.0 |
| E35D/M47V/K89N | 2851 | 39282 | 1.0 | 507 | 0.7 | 4336 | 33.9 | 8.6 |
| E35D/N48K/V68M | 2852 | 33583 | 0.8 | 642 | 0.9 | 17733 | 138.5 | 27.6 |
| E35D/N48K/K89N | 2853 | 34727 | 0.9 | 411 | 0.6 | 5766 | 45.0 | 14.0 |
| E35D/V68M/K89N | 2854 | 24838 | 0.6 | 1191 | 1.7 | 10422 | 81.4 | 8.8 |
| M47V/N48K/V68M | 2855 | 34612 | 0.9 | 641 | 0.9 | 14464 | 113.0 | 22.6 |
| M47V/N48K/K89N | 2856 | 42071 | 1.0 | 366 | 0.5 | 2366 | 18.5 | 6.5 |
| M47V/V68M/K89N | 2857 | 24787 | 0.6 | 1324 | 1.9 | 11806 | 92.2 | 8.9 |
| N48K/V68M/K89N | 2858 | 19129 | 0.5 | 1176 | 1.7 | 11464 | 89.6 | 9.7 |
| E35D/M47V/N48K/V68M | 2764 | 32913 | 0.8 | 789 | 1.1 | 23479 | 183.4 | 29.8 |
| E35D/M47V/N48K/K89N | 2859 | 43756 | 1.1 | 701 | 1.0 | 6669 | 52.1 | 9.5 |
| E35D/M47V/V68M/K89N | 2860 | 29493 | 0.7 | 1610 | 2.3 | 21827 | 170.5 | 13.6 |
| E35D/N48K/V68M/K89N | 2861 | 29772 | 0.7 | 1534 | 2.2 | 17425 | 136.1 | 11.4 |
| M47V/N48K/V68M/K89N | 2862 | 29777 | 0.7 | 1597 | 2.3 | 23666 | 184.9 | 14.8 |
| E35D/D46V/M47V/N48K/ | 2863 | 23880 | 0.6 | 1085 | 1.5 | 25940 | 202.7 | 23.9 |
| V68M | | | | | | | | |
| E35D/D46V/M47V/V68M/ | 2864 | 36463 | 0.9 | 331 | 0.5 | 26290 | 205.4 | 79.4 |
| L85Q | | | | | | | | |
| E35D/D46V/M47V/V68M/ | 2865 | 15124 | 0.4 | 2119 | 3.0 | 21603 | 168.8 | 10.2 |
| K89N | | | | | | | | |
| E35D/M47V/N48K/V68M/ | 2866 | 26104 | 0.6 | 118 | 0.2 | 10479 | 81.9 | 88.8 |
| L85Q | | | | | | | | |
| E35D/M47V/N48K/V68M/ | 2250 | 20884 | 0.5 | 1348 | 1.9 | 14800 | 115.6 | 11.0 |
| K89N | | | | | | | | |
| E35D/M47V/V68M/L85Q/ | 2867 | 30276 | 0.7 | 246 | 0.3 | 12085 | 94.4 | 49.1 |
| K89N | | | | | | | | |
| WT CD80 ECD-Fc | | 40376 | 1.0 | 709 | 1.0 | 128 | 1.0 | 0.2 |
| (Abcam) | | | | | | | | |
| Fc Control | 1520 | 52 | 0.0 | 12.7 | 0.0 | 44 | 0.3 | 3.5 |

TABLE E20
Flow Binding to Jurkats (CD28) and CHO cells stably expressing CTLA4 or PD-
L1

| | | CTLA4 | | CD28 | | PD-L1 | | |
|---|---|---|---|---|---|---|---|---|
| | | | Fold | | Fold | | Fold | |
| | | MFI | change | MFI | change | MFI | change | Ratio |
| | | at | to | at | to | at | to | of |
| | SEQ ID | 33.3 | WT | 33.3 | WT | 33.3 | WT | PDL1: |
| Mutation(s) | NO (IgV) | nM | CD80 | nM | CD80 | nM | CD80 | CD28 |
| A26E/E35D/M47L/V68M/ | 1083 | 21749 | 16.0 | 2211 | 50.4 | 30232 | 693.4 | 13.7 |
| A71G/D90G | | | | | | | | |
| H18Y/E35D/M47L/V68M/ | 1084 | 19892 | 14.6 | 2793 | 63.6 | 29944 | 686.8 | 10.7 |
| A71G/D90G | | | | | | | | |
| H18Y/A26E/M47L/V68M/ | 1085 | 121 | 0.1 | 2556 | 58.2 | 31716 | 727.4 | 12.4 |
| A71G/D90G | | | | | | | | |
| H18Y/A26E/E35D/V68M/ | 1086 | 23386 | 17.2 | 1757 | 40.0 | 28683 | 657.9 | 16.3 |
| A71G/D90G | | | | | | | | |
| H18Y/A26E/E35D/M47L/ | 1087 | 21215 | 15.6 | 1099 | 25.0 | 16926 | 388.2 | 15.4 |
| A71G/D90G | | | | | | | | |
| H18Y/A26E/E35D/M47L/ | 1088 | 24855 | 18.3 | 2675 | 60.9 | 25217 | 578.4 | 9.4 |
| V68M/D90G | | | | | | | | |
| H18Y/A26E/E35D/M47L/ | 1089 | 25404 | 18.7 | 526 | 12.0 | 28546 | 654.7 | 54.3 |
| V68M/A71G | | | | | | | | |
| E35D/M47L/V68M/A71G/ | 1090 | 26007 | 19.1 | 3072 | 70.0 | 29377 | 673.8 | 9.6 |
| D90G | | | | | | | | |
| H18Y/M47L/V68M/A71G/ | 1091 | 22235 | 16.4 | 3184 | 72.5 | 29517 | 677.0 | 9.3 |
| D90G | | | | | | | | |
| H18Y/A26E/V68M/A71G/ | 1092 | 18305 | 13.5 | 2683 | 61.1 | 27872 | 639.3 | 10.4 |
| D90G | | | | | | | | |
| H18Y/A26E/E35D/A71G/ | 1093 | −100 | −0.1 | 1075 | 24.5 | 14822 | 340.0 | 13.8 |
| D90G | | | | | | | | |
| H18Y/A26E/E35D/M47L/ | 1094 | 19736 | 14.5 | 1379 | 31.4 | 12698 | 291.2 | 9.2 |
| D90G | | | | | | | | |
| H18Y/A26E/E35D/M47L/ | 1095 | 20015 | 14.7 | 626 | 14.3 | 24683 | 566.1 | 39.4 |
| V68M | | | | | | | | |
| A26E/M47L/V68M/A71G/ | 1096 | 21807 | 16.0 | 2790 | 63.6 | 28139 | 645.4 | 10.1 |
| D90G | | | | | | | | |
| A26E/E35D/V68M/A71G/ | 1097 | 23286 | 17.1 | 2102 | 47.9 | 26510 | 608.0 | 12.6 |
| D90G | | | | | | | | |
| A26E/E35D/M47L/A71G/ | 1098 | 22127 | 16.3 | 1272 | 29.0 | 14550 | 333.7 | 11.4 |
| D90G | | | | | | | | |
| A26E/E35D/M47L/V68M/ | 1099 | 26698 | 19.6 | 2908 | 66.2 | 24978 | 572.9 | 8.6 |
| D90G | | | | | | | | |
| A26E/E35D/M47L/V68M/ | 1100 | 24587 | 18.1 | 417 | 9.5 | 27806 | 637.8 | 66.7 |
| A71G | | | | | | | | |
| H18Y/E35D/V68M/A71G/ | 1101 | 24335 | 17.9 | 2724 | 62.1 | 30088 | 690.1 | 11.0 |
| D90G | | | | | | | | |
| H18Y/E35D/M47L/A71G/ | 1102 | 22983 | 16.9 | 1273 | 29.0 | 13327 | 305.7 | 10.5 |
| D90G | | | | | | | | |
| H18Y/E35D/M47L/V68M/ | 1103 | 22834 | 16.8 | 3389 | 77.2 | 27410 | 628.7 | 8.1 |
| D90G | | | | | | | | |
| H18Y/E35D/M47L/V68M/ | 1104 | 23667 | 17.4 | 928 | 21.1 | 30377 | 696.7 | 32.7 |
| A71G | | | | | | | | |
| H18Y/A26E/M47L/A71G/ | 1105 | 25420 | 18.7 | 2047 | 46.6 | 17737 | 406.8 | 8.7 |
| D90G | | | | | | | | |
| H18Y/A26E/M47L/V68M/ | 1106 | 28649 | 21.1 | 32 | 0.7 | 23594 | 541.1 | 737.3 |
| D90G | | | | | | | | |
| H18Y/A26E/M47L/V68M/ | 1107 | 21742 | 16.0 | 544 | 12.4 | 29730 | 681.9 | 54.7 |
| A71G | | | | | | | | |
| H18Y/A26E/E35D/V68M/ | 1108 | 19331 | 14.2 | 2584 | 58.9 | 23206 | 532.2 | 9.0 |
| D90G | | | | | | | | |
| H18Y/A26E/E35D/V68M/ | 1109 | 19394 | 14.3 | 394 | 9.0 | 27476 | 630.2 | 69.7 |
| A71G | | | | | | | | |
| H18Y/A26E/E35D/M47L/ | 1110 | 19353 | 14.2 | 379 | 8.6 | 16887 | 387.3 | 44.6 |
| A71G | | | | | | | | |
| M47L/V68M/A71G/D90G | 1111 | 17418 | 12.8 | 3610 | 82.2 | 31114 | 713.6 | 8.6 |
| H18Y/V68M/A71G/D90G | 1112 | 22321 | 16.4 | 3414 | 77.8 | 30670 | 703.4 | 9.0 |
| H18Y/A26E/A71G/D90G | 1113 | 19878 | 14.6 | 2001 | 45.6 | 15491 | 355.3 | 7.7 |
| H18Y/A26E/E35D/D90G | 1114 | 22813 | 16.8 | 46.5 | 1.1 | 10019 | 229.8 | 215.5 |
| H18Y/A26E/E35D/M47L | 1115 | 23990 | 17.7 | 324 | 7.4 | 9951 | 228.2 | 30.7 |
| E35D/V68M/A71G/D90G | 1116 | 23290 | 17.1 | 2843 | 64.8 | 28005 | 642.3 | 9.9 |
| E35D/M47L/A71G/D90G | 1117 | 20921 | 15.4 | 1331 | 30.3 | 12073 | 276.9 | 9.1 |
| E35D/M47L/V68M/D90G | 1118 | 27607 | 20.3 | 3414 | 77.8 | 23482 | 538.6 | 6.9 |
| E35D/M47L/V68M/A71G | 1119 | 24656 | 18.1 | 806 | 18.4 | 27872 | 639.3 | 34.6 |
| A26E/V68M/A71G/D90G | 1120 | 8666 | 6.4 | 1194 | 27.2 | 3195 | 73.3 | 2.7 |
| A26E/M47L/A71G/D90G | 1121 | 21955 | 16.2 | 1955 | 44.5 | 13204 | 302.8 | 6.8 |
| A26E/M47L/V68M/D90G | 1122 | 21900 | 16.1 | 2583 | 58.8 | 10626 | 243.7 | 4.1 |
| A26E/M47L/V68M/A71G | 1123 | 3227 | 2.4 | 98.7 | 2.2 | 1667 | 38.2 | 16.9 |
| A26E/E35D/V68M/D90G | 1125 | 13879 | 10.2 | 1683 | 38.3 | 6987 | 160.3 | 4.2 |
| A26E/E35D/V68M/A71G | 1126 | 11791 | 8.7 | 135 | 3.1 | 12611 | 289.2 | 93.4 |
| A26E/E35D/M47L/D90G | 1127 | 18167 | 13.4 | 1550 | 35.3 | 9577 | 219.7 | 6.2 |
| A26E/E35D/M47L/A71G | 471 | 20645 | 15.2 | 236 | 5.4 | 11666 | 267.6 | 49.4 |
| H18Y/M47L/A71G/D90G | 1129 | 18162 | 13.4 | 1601 | 36.5 | 10796 | 247.6 | 6.7 |
| H18Y/M47L/V68M/D90G | 1130 | 19006 | 14.0 | 3795 | 86.4 | 21768 | 499.3 | 5.7 |
| H18Y/M47L/V68M/A71G | 1131 | 21298 | 15.7 | 1192 | 27.2 | 28478 | 653.2 | 23.9 |
| H18Y/E35D/A71G/D90G | 1132 | 25886 | 19.0 | 1310 | 29.8 | 8524 | 195.5 | 6.5 |
| H18Y/E35D/M47L/A71G | 1136 | 22368 | 16.5 | 604 | 13.8 | 11881 | 272.5 | 19.7 |
| H18Y/A26E/V68M/D90G | 1138 | 25794 | 19.0 | 2394 | 54.5 | 12845 | 294.6 | 5.4 |
| H18Y/A26E/V68M/A71G | 1139 | 11323 | 8.3 | 99.4 | 2.3 | 6866 | 157.5 | 69.1 |
| H18Y/A26E/M47L/D90G | 1140 | 23485 | 17.3 | 2858 | 65.1 | 8933 | 204.9 | 3.1 |
| H18Y/A26E/M47L/A71G | 1141 | 22108 | 16.3 | 611 | 13.9 | 15563 | 356.9 | 25.5 |
| H18Y/A26E/E35D/V68M | 1144 | 20929 | 15.4 | 372 | 8.5 | 17904 | 410.6 | 48.1 |
| H18Y/A26E/E35D/M47L/ | 491 | 18244 | 13.4 | 1836 | 41.8 | 29167 | 669.0 | 15.9 |
| V68M/A71G/D90G | | | | | | | | |
| CD80 WT IgV-Fc | 150 | 1359 | 1.0 | 43.9 | 1.0 | 43.6 | 1.0 | 1.0 |
| CD80 ECD-Fc | 2 | 19552 | 14.4 | 42.3 | 1.0 | 6377 | 146.3 | 150.8 |
| Fc Control | 1520 | 37.9 | 0.0 | 15.4 | 0.4 | 77.1 | 1.8 | 5.0 |

TABLE E21
Jurkat/IL2 + CHO/OKT3/PD-L1 Reporter Assay: Relative
Luciferase Units (RLU)

| | | | Fold Increase |
|---|---|---|---|
| | SEQ | CD80 | over |
| | ID | Conc | WT CD80- |
| Mutation(s) | NO | 5.0 nM | IgV-Fe |
| E35D | 198 | 368 | 3.2 |
| M47L | 1029 | 530 | 4.6 |
| V68M | 1030 | 130 | 1.1 |
| L85Q | 1031 | 132 | 1.1 |
| E35D/D46V | 1032 | 609 | 5.3 |
| E35D/M47L | 208 | 603 | 5.2 |
| D46V/M47L | 1035 | 773 | 6.7 |
| D46V/V68M | 1036 | 292 | 2.5 |
| D46V/L85Q | 1037 | 342 | 3.0 |
| M47L/L85Q | 1039 | 416 | 3.6 |
| V68M/L85Q | 1040 | 146 | 1.3 |
| E35D/D46V/M47L | 1041 | 746 | 6.5 |
| E35D/D46V/V68M | 1042 | 799 | 6.9 |
| E35D/D46V/L85Q | 1043 | 410 | 3.6 |
| E35D/M47L/V68M | 971 | 749 | 6.5 |
| E35D/M47L/L85Q | 418 | 177 | 1.5 |
| E35D/V68M/L85Q | 1044 | 511 | 4.4 |
| D46V/M47L/V68M | 1045 | 724 | 6.3 |
| D46V/M47L/L85Q | 1046 | 598 | 5.2 |
| D46V/V68M/L85Q | 1047 | 267 | 2.3 |
| M47L/V68M/L85Q | 1048 | 238 | 2.1 |
| E35D/D46V/M47L/V68M | 976 | 681 | 5.9 |
| E35D/D46V/M47L/L85Q | 1049 | 481 | 4.2 |
| E35D/D46V/V68M/L85Q | 1050 | 864 | 7.5 |
| E35D/M47L/V68M/L85Q | 1051 | 890 | 7.7 |
| D46V/M47L/V68M/L85Q | 1052 | 654 | 5.7 |
| E35D/D46V/M47L/V68M/L85Q | 975 | 712 | 6.2 |
| M47V | 1053 | 445 | 3.9 |
| N48K | 1054 | 160 | 1.4 |
| K89N | 1055 | 116 | 1.0 |
| E35D/M47V | 944 | 543 | 4.7 |
| E35D/N48K | 1056 | 590 | 5.1 |
| E35D/K89N | 1057 | 293 | 2.5 |
| M47V/N48K | 1058 | 490 | 4.3 |
| M47V/V68M | 1059 | 553 | 4.8 |
| M47V/K89N | 1060 | 312 | 2.7 |
| N48K/V68M | 1061 | 127 | 1.1 |
| N48K/K89N | 1062 | 127 | 1.1 |
| V68M/K89N | 1063 | 100 | 0.9 |
| E35D/M47V/N48K | 1064 | 561 | 4.9 |
| E35D/M47V/V68M | 1065 | 841 | 7.3 |
| E35D/M47V/K89N | 1066 | 668 | 5.8 |
| E35D/N48K/V68M | 1067 | 721 | 6.3 |
| E35D/N48K/K89N | 1068 | 719 | 6.3 |
| E35D/V68M/K89N | 1069 | 537 | 4.7 |
| M47V/N48K/V68M | 1070 | 664 | 5.8 |
| M47V/N48K/K89N | 1071 | 472 | 4.1 |
| M47V/V68M/K89N | 1072 | 862 | 7.5 |
| N48K/V68M/K89N | 1073 | 614 | 5.3 |
| E35D/M47V/N48K/V68M | 979 | 747 | 6.5 |
| E35D/M47V/N48K/K89N | 1074 | 814 | 7.1 |
| E35D/M47V/V68M/K89N | 1075 | 779 | 6.8 |
| E35D/N48K/V68M/K89N | 1076 | 772 | 6.7 |
| M47V/N48K/V68M/K89N | 1077 | 671 | 5.8 |
| E35D/D46V/M47V/N48K/V68M | 1078 | 696 | 6.1 |
| E35D/D46V/M47V/V68M/L85Q | 1079 | 980 | 8.5 |
| E35D/D46V/M47V/V68M/K89N | 1080 | 817 | 7.1 |
| E35D/M47V/N48K/V68M/L85Q | 1081 | 907 | 7.9 |
| E35D/M47V/N48K/V68M/K89N | 2250 | 767 | 6.7 |
| E35D/M47V/V68M/L85Q/K89N | 1082 | 854 | 7.4 |
| CD80 WT IgV-Fc | 150 | 115 | 1.0 |
| CD80 ECD-Fc | 465 | 131 | 1.1 |
| Fc Control | 1520 | 97 | 0.8 |

TABLE E22
Jurkat/IL2 + CHO/OKT3/PD-L1 Reporter Assay: Relative
Luciferase Units (RLU)

| | | | Fold Increase |
|---|---|---|---|
| | SEQ | CD80 | over WT |
| | ID | Conc | CD80- |
| Mutation(s) | NO | 5.0 nM | IgV-Fe |
| A26E/E35D/M47L/V68M/A71G/D90G | 1083 | 1117 | 2.86 |
| H18Y/E35D/M47L/V68M/A71G/D90G | 1084 | 1028 | 2.64 |
| H18Y/A26E/M47L/V68M/A71G/D90G | 1085 | 853 | 2.19 |
| H18Y/A26E/E35D/V68M/A71G/D90G | 1086 | 940 | 2.41 |
| H18Y/A26E/E35D/M47L/A71G/D90G | 1087 | 1015 | 2.60 |
| H18Y/A26E/E35D/M47L/V68M/D90G | 1088 | 893 | 2.29 |
| H18Y/A26E/E35D/M47L/V68M/A71G | 1089 | 976 | 2.50 |
| E35D/M47L/V68M/A71G/D90G | 1090 | 1041 | 2.67 |
| H18Y/M47L/V68M/A71G/D90G | 1091 | 986 | 2.53 |
| H18Y/A26E/V68M/A71G/D90G | 1092 | 974 | 2.50 |
| H18Y/A26E/E35D/A71G/D90G | 1093 | 956 | 2.45 |
| H18Y/A26E/E35D/M47L/D90G | 1094 | 925 | 2.37 |
| H18Y/A26E/E35D/M47L/V68M | 1095 | 895 | 2.29 |
| A26E/M47L/V68M/A71G/D90G | 1096 | 793 | 2.03 |
| A26E/E35D/V68M/A71G/D90G | 1097 | 912 | 2.34 |
| A26E/E35D/M47L/A71G/D90G | 1098 | 1132 | 2.90 |
| A26E/E35D/M47L/V68M/D90G | 1099 | 1091 | 2.80 |
| A26E/E35D/M47L/V68M/A71G | 1100 | 1010 | 2.59 |
| H18Y/E35D/V68M/A71G/D90G | 1101 | 815 | 2.09 |
| H18Y/E35D/M47L/A71G/D90G | 1102 | 851 | 2.18 |
| H18Y/E35D/M47L/V68M/D90G | 1103 | 852 | 2.18 |
| H18Y/E35D/M47L/V68M/A71G | 1104 | 853 | 2.19 |
| H18Y/A26E/M47L/A71G/D90G | 1105 | 1036 | 2.66 |
| H18Y/A26E/M47L/V68M/D90G | 1106 | 1075 | 2.76 |
| H18Y/A26E/M47L/V68M/A71G | 1107 | 1160 | 2.97 |
| H18Y/A26E/E35D/V68M/D90G | 1108 | 1049 | 2.69 |
| H18Y/A26E/E35D/V68M/A71G | 1109 | 961 | 2.46 |
| H18Y/A26E/E35D/M47L/A71G | 1110 | 944 | 2.42 |
| M47L/V68M/A71G/D90G | 1111 | 771 | 1.98 |
| H18Y/V68M/A71G/D90G | 1112 | 797 | 2.04 |
| H18Y/A26E/A71G/D90G | 1113 | 933 | 2.39 |
| H18Y/A26E/E35D/D90G | 1114 | 948 | 2.43 |
| H18Y/A26E/E35D/M47L | 1115 | 1208 | 3.10 |
| E35D/V68M/A71G/D90G | 1116 | 990 | 2.54 |
| E35D/M47L/A71G/D90G | 1117 | 784 | 2.01 |
| E35D/M47L/V68M/D90G | 1118 | 711 | 1.82 |
| E35D/M47L/V68M/A71G | 1119 | 745 | 1.91 |
| A26E/V68M/A71G/D90G | 1120 | 590 | 1.51 |
| A26E/M47L/A71G/D90G | 1121 | 827 | 2.12 |
| A26E/M47L/V68M/D90G | 1122 | 821 | 2.11 |
| A26E/M47L/V68M/A71G | 1123 | 517 | 1.33 |
| A26E/E35D/V68M/D90G | 1125 | 871 | 2.23 |
| A26E/E35D/V68M/A71G | 1126 | 839 | 2.15 |
| A26E/E35D/M47L/D90G | 1127 | 843 | 2.16 |
| A26E/E35D/M47L/A71G | 471 | 766 | 1.96 |
| H18Y/M47L/A71G/D90G | 1129 | 675 | 1.73 |
| H18Y/M47L/V68M/D90G | 1130 | 834 | 2.14 |
| H18Y/M47L/V68M/A71G | 1131 | 881 | 2.26 |
| H18Y/E35D/A71G/D90G | 1132 | 1487 | 3.81 |
| H18Y/E35D/M47L/A71G | 1136 | 1387 | 3.56 |
| H18Y/A26E/V68M/D90G | 1138 | 1131 | 2.90 |
| H18Y/A26E/V68M/A71G | 1139 | 469 | 1.20 |
| H18Y/A26E/M47L/D90G | 1140 | 1159 | 2.97 |
| H18Y/A26E/M47L/A71G | 1141 | 1107 | 2.84 |
| H18Y/A26E/E35D/V68M | 1144 | 1214 | 3.11 |
| CD80 WT IgV-Fc | 150 | 390 | 1.00 |

### Example 12

### Variant Optimization Via NNK Library Selection

Additional variant CD80 IgV domain-containing molecules were generated with combinations of mutations at positions 18, 26, 35, 47, 48, 68, 71, 85, 88, 90 and 93 with reference to positions set forth in SEQ ID NOs: 465, 491, and 495. The variants were generated from an NNK library at the selected positions, where N=A,G,C or T and K=T or G, such that the degenerate codons encode all potential amino acids, but prevent the encoding of two stop residues TAA and TGA. The NNK containing DNA was introduced into yeast substantially as described in Example 2 to generate yeast libraries. The libraries were used to select yeast expressing affinity modified variants of CD80 substantially as described in Example 3.

Outputs from three rounds of FACS selections with rhPD-L1-Fc substantially as described in Example 4 were further formatted, selected and expressed as inert Fc-fusion proteins substantially as described in Example 5. The Fc-fusion proteins were tested for binding, substantially as described in Example 7, and bioactivity, substantially described in Example 9. Binding and bioactivity of wild-type CD80 ECD-Fc (inert), wild-type CD80 IgV-Fc (inert), H18Y/A26E/E35D/M47L/V68M/A71G/D90G (SEQ ID NO: 491) CD80 IgV-Fc (inert), and inert Fc alone were also measured for reference. Results from the binding and activity studies are provided in Tables E23 and E24, respectively.

TABLE E23
Flow Binding to Jurkat (CD28) and CHO cells stably expressing CTLA4 or PD-L1

| | | CTLA4 | | CD28 | | PD-L1 | | |
|---|---|---|---|---|---|---|---|---|
| | | | Fold | | Fold | | Fold | |
| | | MFI | change | MFI | change | MFI | change | Ratio |
| | | at | to | at | to | at | to | of |
| | SEQ ID | 33.3 | WT | 33.3 | WT | 33.3 | WT | PDL1: |
| Mutation(s) | NO (IgV) | nM | CD80 | nM | CD80 | nM | CD80 | CD28 |
| H18Y/E35D/M47V/V68M/ | 1207 | 23650 | 17.1 | 3227 | 31.6 | 64919 | 393.4 | 20.1 |
| A71G | | | | | | | | |
| H18C/A26P/E35D/M47L/ | 1208 | 23371 | 16.9 | 1906 | 18.7 | 67010 | 406.1 | 35.2 |
| V68M/A71G | | | | | | | | |
| H18I/A26P/E35D/M47V/ | 1209 | 21923 | 15.8 | 2573 | 25.2 | 64919 | 393.4 | 25.2 |
| V68M/A71G | | | | | | | | |
| H18L/A26N/D46E/V68M/ | 1210 | 17045 | 12.3 | 7253 | 71.1 | 67999 | 412.1 | 9.4 |
| A71G/D90G | | | | | | | | |
| H18L/E35D/M47V/V68M/ | 1211 | 20280 | 14.7 | 6349 | 62.2 | 64761 | 392.5 | 10.2 |
| A71G/D90G | | | | | | | | |
| H18T/A26N/E35D/M47L/ | 1212 | 20911 | 15.1 | 1366 | 13.4 | 68498 | 415.1 | 50.1 |
| V68M/A71G | | | | | | | | |
| H18V/A26K/E35D/M47L/ | 1213 | 22932 | 16.6 | 3641 | 35.7 | 67338 | 408.1 | 18.5 |
| V68M/A71G | | | | | | | | |
| H18V/A26N/E35D/M47V/ | 1214 | 22395 | 16.2 | 1297 | 12.7 | 68165 | 413.1 | 52.6 |
| V68M/A71G | | | | | | | | |
| H18V/A26P/E35D/M47V/ | 1215 | 13669 | 9.9 | 2253 | 22.1 | 55417 | 335.9 | 24.6 |
| V68L/A71G | | | | | | | | |
| H18V/A26P/E35D/M47L/ | 1216 | 16192 | 11.7 | 2452 | 24.0 | 52405 | 317.6 | 21.4 |
| V68M/A71G | | | | | | | | |
| H18V/E35D/M47V/V68M/ | 1217 | 16769 | 12.1 | 2115 | 20.7 | 43588 | 264.2 | 20.6 |
| A71G/D90G | | | | | | | | |
| H18Y/A26P/E35D/M471/ | 1218 | 12156 | 8.8 | 5125 | 50.2 | 54482 | 330.2 | 10.6 |
| V68M/A71G | | | | | | | | |
| H18Y/A26P/E35D/M47V/ | 1219 | 17904 | 12.9 | 6911 | 67.8 | 51521 | 312.2 | 7.5 |
| V68M/A71G | | | | | | | | |
| H18Y/E35D/M47V/V68L/ | 1220 | 16458 | 11.9 | 2549 | 25.0 | 47905 | 290.3 | 18.8 |
| A71G/D90G | | | | | | | | |
| H18Y/E35D/M47V/V68M/ | 1221 | 17165 | 12.4 | 6792 | 66.6 | 52151 | 316.1 | 7.7 |
| A71G/D90G | | | | | | | | |
| A26P/E35D/M47I/V68M/ | 1222 | 19761 | 14.3 | 8189 | 80.3 | 54747 | 331.8 | 6.7 |
| A71G/D90G | | | | | | | | |
| H18V/A26G/E35D/M47V/ | 1223 | 25398 | 18.4 | 8189 | 80.3 | 66198 | 401.2 | 8.1 |
| V68M/A71G/D90G | | | | | | | | |
| H18V/A26S/E35D/M47L/ | 1224 | 24919 | 18.0 | 8063 | 79.0 | 73884 | 447.8 | 9.2 |
| V68M/A71G/D90G | | | | | | | | |
| H18V/A26R/E35D/M47L/ | 1225 | 23151 | 16.7 | 9620 | 94.3 | 73166 | 443.4 | 7.6 |
| V68M/A71G/D90G | | | | | | | | |
| H18V/A26D/E35D/M47V/ | 1226 | 22132 | 16.0 | 6253 | 61.3 | 67503 | 409.1 | 10.8 |
| V68M/A71G/D90G | | | | | | | | |
| H18V/A26Q/E35D/M47V/ | 1227 | 17654 | 12.8 | 3126 | 30.6 | 33597 | 203.6 | 10.7 |
| V68L/A71G/D90G | | | | | | | | |
| H18A/A26P/E35D/M47L/ | 1228 | 23763 | 17.2 | 4731 | 46.4 | 33436 | 202.6 | 7.1 |
| V68M/A71G/D90G | | | | | | | | |
| H18A/A26N/E35D/M47L/ | 1229 | 21360 | 15.4 | 4913 | 48.2 | 36284 | 219.9 | 7.4 |
| V68M/A71G/D90G | | | | | | | | |
| H18F/A26P/E35D/M47I/ | 1230 | 23932 | 17.3 | 4801 | 47.1 | 32253 | 195.5 | 6.7 |
| V68M/A71G/D90G | | | | | | | | |
| H18F/A26H/E35D/M47L/ | 1231 | 16420 | 11.9 | 8392 | 82.3 | 20666 | 125.2 | 2.5 |
| V68M/A71G/D90G | | | | | | | | |
| H18F/A26N/E35D/M47V/ | 1232 | 15206 | 11.0 | 3170 | 31.1 | 22395 | 135.7 | 7.1 |
| V68M/A71G/D90K | | | | | | | | |
| H18Y/A26N/E35D/M47F/ | 1233 | 14618 | 10.6 | 82.2 | 0.8 | 26510 | 160.7 | 322.5 |
| V68M/A71G/D90G | | | | | | | | |
| H18Y/A26P/E35D/M47Y/ | 1234 | 8281 | 6.0 | 1818 | 17.8 | 27280 | 165.3 | 15.0 |
| V68I/A71G/D90G | | | | | | | | |
| H18Y/A26Q/E35D/M47T/ | 1235 | 16652 | 12.0 | 6733 | 66.0 | 24450 | 148.2 | 3.6 |
| V68M/A71G/D90G | | | | | | | | |
| H18R/A26P/E35D/D46N/ | 1236 | 17327 | 12.5 | 18589 | 182.2 | 29306 | 177.6 | 1.6 |
| M47V/V68M/A71G/D90P | | | | | | | | |
| H18F/A26D/E35D/D46E/ | 1237 | 17205 | 12.4 | 6028 | 59.1 | 27541 | 166.9 | 4.6 |
| M47T/V68M/A71G/D90G | | | | | | | | |
| H18Y/A26E/E35D/M47L/ | 491 | 21512 | 15.5 | 5202 | 51.0 | 35251 | 213.6 | 6.8 |
| V68M/A71G/D90G | | | | | | | | |
| CD80 WT IgV-Fc | | 1384 | 1.0 | 102 | 1.0 | 165 | 1.0 | 1.6 |
| CD80 WT ECD-Fc | | 17862 | 12.9 | 57.8 | 0.6 | 161 | 1.0 | 2.8 |
| Fc Control | | 194 | 0.1 | 81 | 0.8 | 185 | 1.1 | 2.3 |

TABLE E24
Jurkat/IL2 + CHO/OKT3/PD-L1 Reporter Assay: Relative
Luciferase Units (RLU)

| | | | Fold |
|---|---|---|---|
| | | | Increase |
| | SEQ | CD80 | over WT |
| | ID | Conc | CD80- |
| CD80 Mutation(s) | NO: | 5.0 nM | IgV-Fe |
| H18Y/E35D/M47V/V68M/A71G | 1207 | 963 | 5.6 |
| H18C/A26P/E35D/M47L/V68M/A71G | 1208 | 936 | 5.5 |
| H181/A26P/E35D/M47V/V68M/A71G | 1209 | 916 | 5.4 |
| H18L/A26N/D46E/V68M/A71G/D90G | 1210 | 815 | 4.8 |
| H18L/E35D/M47V/V68M/A71G/D90G | 1211 | 910 | 5.3 |
| H18T/A26N/E35D/M47L/V68M/A71G | 1212 | 1053 | 6.2 |
| H18V/A26K/E35D/M47L/V68M/A71G | 1213 | 957 | 5.6 |
| H18V/A26N/E35D/M47V/V68M/A71G | 1214 | 985 | 5.8 |
| H18V/A26P/E35D/M47V/V68L/A71G | 1215 | 881 | 5.2 |
| H18V/A26P/E35D/M47L/V68M/A71G | 1216 | 808 | 4.7 |
| H18V/E35D/M47V/V68M/A71G/D90G | 1217 | 854 | 5.0 |
| H18Y/A26P/E35D/M47T/V68M/A71G | 1218 | 761 | 4.5 |
| H18Y/A26P/E35D/M47V/V68M/A71G | 1219 | 821 | 4.8 |
| H18Y/E35D/M47V/V68L/A71G/D90G | 1220 | 862 | 5.0 |
| H18Y/E35D/M47V/V68M/A71G/D90G | 1221 | 825 | 4.8 |
| A26P/E35D/M47I/V68M/A71G/D90G | 1222 | 823 | 4.8 |
| H18V/A26G/E35D/M47V/V68M/A71G/D90G | 1223 | 907 | 5.3 |
| H18V/A26S/E35D/M47L/V68M/A71G/D90G | 1224 | 883 | 5.2 |
| H18V/A26R/E35D/M47L/V68M/A71G/D90G | 1225 | 738 | 4.3 |
| H18V/A26D/E35D/M47V/V68M/A71G/D90G | 1226 | 771 | 4.5 |
| H18V/A26Q/E35D/M47V/V68L/A71G/D90G | 1227 | 795 | 4.6 |
| H18A/A26P/E35D/M47L/V68M/A71G/D90G | 1228 | 857 | 5.0 |
| H18A/A26N/E35D/M47L/V68M/A71G/D90G | 1229 | 1054 | 6.2 |
| H18F/A26P/E35D/M47I/V68M/A71G/D90G | 1230 | 926 | 5.4 |
| H18F/A26H/E35D/M47L/V68M/A71G/D90G | 1231 | 907 | 5.3 |
| H18F/A26N/E35D/M47V/V68M/A71G/D90K | 1232 | 919 | 5.4 |
| H18Y/A26N/E35D/M47F/V68M/A71G/D90G | 1233 | 911 | 5.3 |
| H18Y/A26P/E35D/M47Y/V68I/A71G/D90G | 1234 | 865 | 5.1 |
| H18Y/A26Q/E35D/M47T/V68M/A71G/D90G | 1235 | 994 | 5.8 |
| H18R/A26P/E35D/D46N/M47V/V68M/ | 1236 | 972 | 5.7 |
| A71G/D90P | | | |
| H18F/A26D/E35D/D46E/M47T/V68M/ | 1237 | 833 | 4.9 |
| A71G/D90G | | | |
| H18Y/A26E/E35D/M47L/V68M/A71G/D90G | 491 | 912 | 5.3 |
| CD80 WT IgV-Fc | 150 | 171 | 1.0 |
| CD80 WT ECD-Fc | 2 | 159 | 0.9 |
| Fc Control | 1520 | 129 | 0.8 |

### Example 13

### CD80 IgV-Fc Linker Variants

CD80 IgV-Fc variants were constructed with different linking regions (linkers) between the IgV and Fc domains and binding and/or bioactivity was assessed. Fusion proteins, containing CD80 E35D/M47V/N48K/V68M/K89N IgV-Fc and E35D/D46V/M47L/V68M/L85Q/E88D IgV-Fc proteins, were generated containing EAAAK (SEQ ID NO: 1241), (EAAAK)₃ (SEQ ID NO: 1242), GS(G₄S)₃ (SEQ ID NO: 1243), GS(G₄S)₅ (SEQ ID NO: 1244) linkers.

CD80 IgV-Fc proteins were also generated that contained the E35D/M47V/N48K/V68M/K89N or E35D/D46V/M47L/V68M/L85Q/E88D modifications in a CD80 IgV backbone sequence that was deleted for three amino acids that connect the IgV to IgC in wildtype CD80 (backbone sequence set forth in SEQ ID NO: 1245). The generated variant CD80 IgV was then fused to an inert Fc that was additionally lacking 6 amino acids of the binge region (Fc set forth in SEQ ID NO: 1240). Molecules generated by this strategy were fused directly to the Fc with no additional linker, designated as “delta” linker.

The CD80-IgV-Fc variants were then tested for binding and bioactivity as described in Examples 7 and 9. Binding and bioactivity of wild-type CD80 IgV (SEQ ID NO: 150)-Fc (inert), CD80 ECD (SEQ ID NO:2)-Fc (inert), containing a GSG₄S linker (SEQ ID NO: 1522) and inert Fc alone were also measured for comparison. The results are provided in Tables E25 and E26, respectively.

TABLE E25
Flow Binding to Jurkats (CD28) and CHO cells stably expressing CTLA4 or PD-L1

| | | CTLA4 | | CD28 | | PD-L1 | | |
|---|---|---|---|---|---|---|---|---|
| | | | Fold | | Fold | | Fold | |
| | | | change | | change | | change | |
| | | | to | | to | | to | Ratio of |
| | | MFI at | WT | MFI at | WT | MFI at | WT | PDL1: |
| Mutation(s) | linker | 33.3 nM | CD80 | 33.3 nM | CD80 | 33.3 nM | CD80 | CD28 |
| E35D/M47V/N48K/ | delta | 3091 | 2.6 | 4678 | 83.7 | 20442 | 438.7 | 4 |
| V68M/K89N | EAAAK | 27516 | 23.0 | 2634 | 47.1 | 22862 | 490.6 | 9 |
| | (EAAAK)₃ | 27132 | 22.6 | 1285 | 23.0 | 24476 | 525.2 | 19 |
| | GS(G₄S)₃ | 29793 | 24.9 | 2109 | 37.7 | 24222 | 519.8 | 11 |
| | GS(G4S)₅ | 26994 | 22.5 | 1154 | 20.6 | 22707 | 487.3 | 20 |
| E35D/D46V/M47L/ | delta | 12177 | 10.2 | 4173 | 74.7 | 22538 | 483.6 | 5 |
| V68M/L85Q/E88D | EAAAK | 28959 | 24.2 | 563 | 10.1 | 24821 | 532.6 | 44 |
| | (EAAAK)₃ | 32048 | 26.8 | 197 | 3.5 | 25461 | 546.4 | 129 |
| | GS(G₄S)₃ | 26961 | 22.5 | 267 | 4.8 | 22596 | 484.9 | 85 |
| | GS(G₄S)₅ | 26607 | 22.2 | 143 | 2.6 | 22408 | 480.9 | 157 |
| CD80 WT IgV-Fc | GSG₄S | 1198 | 1.0 | 56 | 1.0 | 47 | 1.0 | 1 |
| CD80 ECD-Fc | GSG₄S | 32735 | 27.3 | 37 | 0.7 | 35 | 0.7 | 1 |
| Inert Fc (control) | N/A | 40 | 0.0 | 20 | 0.3 | 58 | 1.2 | 3 |

TABLE E26
Jurkat/IL2 + CHO/OKT3/PD-L1 Reporter Assay: Relative Luciferase
Units (RLU)

| | | | Fold |
|---|---|---|---|
| | | CD80 | Increase |
| | | Conc | over WT |
| Mutation(s) | linker | 5.0 nM | CD80-IgV-Fe |
| E35D/M47V/N48K/V68M/K89N | delta | 1026 | 2.63 |
| | EAAAK | 1707 | 4.38 |
| | (EAAAK)₃ | 1761 | 4.52 |
| | GS(G₄S)₃ | 1400 | 3.59 |
| | GS(G4S)₅ | 1541 | 3.95 |
| E35D/D46V/M47L/V68M/L85Q/ | delta | 1079 | 2.77 |
| E88D | EAAAK | 1462 | 3.75 |
| | (EAAAK)₃ | 2046 | 5.25 |
| | GS(G₄S)₃ | 1592 | 4.08 |
| | GS(G4S)₅ | 2053 | 5.26 |
| CD80 WT IgV-Fc | GSG₄S | 390 | 1.00 |

### Example 14

### Assessment of Bioactivity of Affinity-Matured CD80 IgSF Domain-Containing Molecules Using a T Cell Stimulation Assay

CD80-IgV-Fc molecules, containing either an inert Fc or effector Fc, were tested at 3 concentrations, 1 nM, 10 nM and 100 nM, for their ability to stimulate T cells in the presence of artificial antigen presenting cells (aAPCs), K562/OKT3+/−PD-L1, as determined by cytokine release (IFN-gamma and IL-2) and T cell proliferation.

100,000 isolated Pan T cells were incubated with 8,000 K562/OKT3 or K562/OTK3/PD-L1 cells (12.5:1 ratio) and 1 nM, 10 nM, or 100 nM CD80-IgV-Fc (effector) or CD80-IgV-Fc (inert). The cell mixture was also incubated with an anti-PD-L1 antibody, wild-type human IgG1, human IgG1 Fc (inert), wild-type CD80 IgV-Fc (effector), wild-type CD80 IgV-Fc (inert), wild-type CD80 ECD-Fc (inert), wild-type CD80 ECD-Fc (effector), or no treatment as controls. IFN-gamma, IL-2 and proliferation were determined after 72 hr. incubation.

Results for IL-2 release are set forth in Table E27. In the first experiment, co-culture of T cells and K562/OKT3 aAPC (not expressing PD-L11), in the presence of certain exemplary assessed variant CD80 IgV-Fc (effector) molecules, resulted in increased IL-2 production. In a second experiment, CD28 costimulation was increased in the presence of certain variant CD80 IgV-Fc (inert) molecules upon co-culture of T cells with K562/OKT3/PD-L1 aAPCs, consistent with PD-L1-dependent CD28 costimulation activity for these variants. CD80 IgV-Fc molecules that poorly bind PD-L1 (i.e. E35G/KA717ED/L72P) did not generate significant costimulation and IL-2 production. In some cases, certain variant CD8 IgV-Fc (effector) molecules, like E35D, were capable of effecting CD28 costimulation only in the presence of PD-L1-expressing aAPC. IFN-gamma and proliferation results were similar to those observed for IL-2 release.

TABLE E27
Primary T Cell CD28 Costimulation via Fc Receptor- or PD-L1-Mediated Cross-
Linking of CD80-IgC-Fc Molecules

| | SEQ | K562/OKT3 (No PD-L1) | | | K562/OKT3/PD-L1 | | |
|---|---|---|---|---|---|---|---|
| | ID NO | CD80-IgV Fc (effector) | | | CD80-IgV Fc (inert) | | |
| CD80 Mutation(s) | (IgV) | 1 nM | 10 nM | 100 nM | 1 nM | 10 nM | 100 nM |
| E35D/M47I | 177 | 11140 | 21590 | 27162 | 244 | 3432 | 8313 |
| A71D/L72V/E95K | 192 | 10593 | 15145 | 21314 | <LOD | <LOD | <LOD |
| E35D | 198 | 7598 | 7988 | 8380 | <LOD | 210 | 2739 |
| E35D/M47I/L70M | 199 | 15695 | 25997 | 25294 | 311 | 6982 | 8393 |
| E35D/M43L/L70M | 201 | 8025 | 7712 | 10496 | <LOD | 52 | 1204 |
| E35D/D46V/L85Q | 203 | 14329 | 21462 | 25421 | <LOD | 102 | 1429 |
| H18Y/A26T/E35D/A71D/L85Q | 207 | 11960 | 20452 | 20581 | <LOD | <LOD | <LOD |
| E35D/M47L | 208 | 14571 | 23581 | 26827 | 268 | 2695 | 7533 |
| E23G/A26S/E35D/T62N/A71D/ | 216 | 15377 | 23462 | 27028 | <LOD | <LOD | 102 |
| L72V/L85M | | | | | | | |
| E35G/K54E/A71D/L72P | 219 | 7032 | 7902 | 8886 | <LOD | <LOD | 59 |
| A26E/E35D/M47L/L85Q | 221 | 6847 | 8318 | 10113 | 72 | 268 | 1455 |
| WT CD80 IgV-Fc (effector) | 150 | 7167 | 7123 | 6203 | Not | Not | Not |
| | | | | | Tested | Tested | Tested |
| WT CD80 IgV-Fc (inert) | 150 | Not | Not | Not | <LOD | 7 | 52 |
| | | Tested | Tested | Tested | | | |
| WT CD80 ECD-Fc (inert) | 2 | 8046 | 7022 | 6481 | Not | Not | Not |
| | (ECD) | | | | Tested | Tested | Tested |
| WT CD80 ECD-Fc (effector) | 2 | 11434 | 20185 | 23118 | 507 | 3114 | 8393 |
| | (ECD) | | | | | | |
| Anti-PD-L1 mAb | | 8220 | 8621 | 6903 | 461 | 821 | 1045 |
| Inert Fc Control | | 7040 | 6335 | 5512 | <LOD | 143 | <LOD |
| WT IgG1 Fc Control | — | 7077 | 6916 | 6258 | Not | Not | Not |
| | | | | | Tested | Tested | Tested |

### Example 15

### Assessment of Variant CD80 Polypeptides Blocking PD-L1/PD-1 Interaction or PD-L1-Dependent Costimulation

A. PD-L1/PD-1 Binding and Blocking

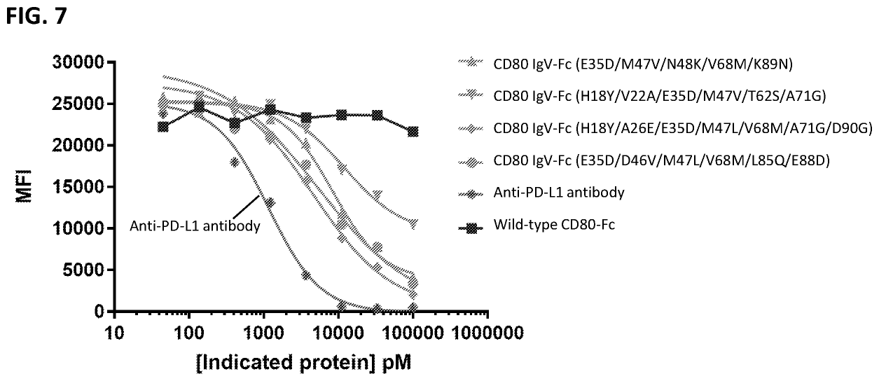

Binding of selected immunomodulatory fusion proteins to cells expressing PD-L1 was assessed to test for blocking of the PD-L1/PD-1 interaction. CHO/PD-L1 cells were stained with a titration of variant CD80 IgV-Fc domain-containing molecules, washed and then incubated with fluorescently conjugated PD-1-Fc. Exemplary variant CD80 IgV domain-containing molecules tested contained E35D/M47V/N48K/V68M/K89N (SEQ ID NO: 465), H18Y/V22A/E35D/M47V/T62S/A71G (SEQ ID NO: 490), H18Y/A26E/E35D/M47L/V68M/A71G/D90G (SEQ ID NO: 491), and E35D/D46V/M47L/V68M/L85Q/E88D (SEQ ID NO: 495). As a control, an anti-PD-L1 antibody and a wild-type CD80 IgV-Fc were also assessed. Samples were acquired on a flow cytometer and MFIs of the fluorescently labeled PD-1 were determined by Flowjo software analysis. As shown in FIG. 7, the exemplary variant CD80 IgV-Fc molecules tested were shown to antagonize or block binding of PD-1 to PD-L1.

B. Activity

Exemplary variant CD80-Fc polypeptides were assessed for their ability to deliver PD-L1 dependent costimulation using Jurkat/IL-2 reporter cells, expressing PD-1, as described above. The Jurkat/IL-2 reporter cells were incubated with K562/OKT3/PD-L1 artificial antigen presenting cells (aAPCs), described above, in the presence of titrated amounts (ranging from 40 pM to 100 nM) of exemplary variant CD80 IgV-Fc polypeptides. Among the exemplary variant CD80 IgV-Fc polypeptides were molecules containing a variant IgV, either E35D/M47V/N48K/V68M/K89N (SEQ ID NO: 465), H18Y/V22A/E35D/M47V/T62S/A71G (SEQ ID NO:490), H18Y/A26E/E35D/M47L/V68M/A71G/D90G (SEQ IN NO: 491), or E35D/D46V/M47L/V68M/L85Q/E88D (SEQ ID NO:495), fused to the exemplary Fc (C220S/L234A/L235E/G237A by EU numbering; SEQ ID NO: 1520), or allotypes thereof. Other tested variant CD80 IgV-Fc polypeptides contained a variant IgV, either E35D/M47I/L70M, SEQ ID NO:199; or E35D/M47L, SEQ ID NO:208) fused to wild-type IgG1 (SEQ ID NO: 1517). As a control, PD-L1-expressing cells were also incubated with wild-Type CD80 IgV-Fc (SEQ ID NO:150) or with an anti-PDL1 antibody (BioLegend USA).

Jurkat/IL-2/PD-1 reporter cells were plated at 100,000 cells per well in Jurkat Assay buffer (RPMI1640+5% FBS). The Jurkat cells were then incubated with test or control proteins for 15 minutes at room temperature. K562/OKT3/PD-L1 cells were then added such that each well had a final ratio of 5:1 Jurkat: K562 cells. Jurkat cells, K562 cells, and test or control proteins were incubated for 5 hours at 37 degrees Celsius in a humidified 5% CO₂ incubation chamber. Plates were then removed from the incubator and acclimated to room temperature for 15 minutes. 100 μL of a cell lysis and luciferase substrate solution (BioGlo luciferase reagent, Promega) were added to each well and the plates were incubated on an orbital shaker for 10 minutes. Luminescence was measured with a 1 second per well integration time using a BioTek Cytation luminometer, and a fold increase in luminescence value (RLU) was determined for each test sample.

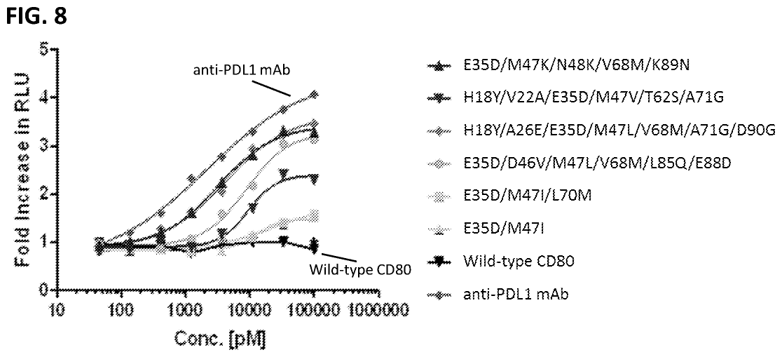

As shown in FIG. 8, the addition of the exemplary assessed variant CD80 IgV-Fc, blocked PD-L1 mediated suppression of the TCR activation and/or agonized CD28, resulting in increased luminescence. Variant molecules identified for increased binding affinity to PD-L1 exhibited greater activity in agonizing T cell activation.

### Example 16

### In Vivo Anti-Tumor Activity of Variant CD80 Polypeptides

A. Anti-Tumor Activity of CD80 Variants

Mouse MC38 tumor cells were stably transfected with human PD-L1 (MC38 hPD-L1) and implanted subcutaneously into C57BU6 mice. An inert Fc control or exemplary variant CD80 IgV-Fc molecules, containing a variant IgV (E35D/M47I/L70M, SEQ ID NO:199; or E35D/M47L, SEQ ID NO:208) fused to either an inert Fc molecule (e.g. SEQ ID NO: 1520, or allotypes thereof) or an Fc molecule capable of mediating effector activity (SEQ ID NO:1517), were injected i.p., 100 μg/mouse, on days 8, 10, 13, 15 and 17 post-implantation. Tumor volume was tracked over time.

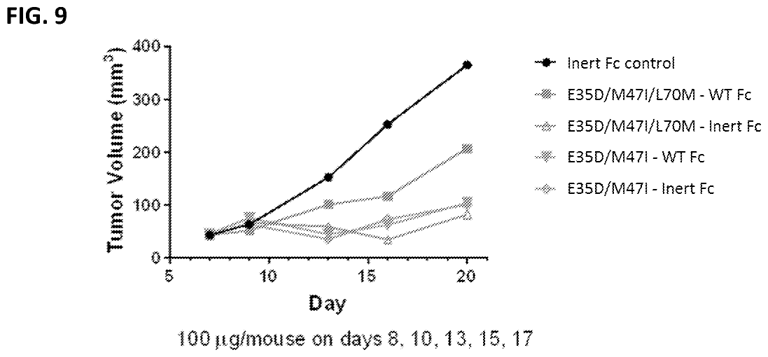

As shown in FIG. 9, suppression of tumor growth was observed in all mice treated with CD80-IgV compared to the Fc control, demonstrating that the variant CD80 IgV-Fc molecules were functionally active in vivo.

B. Dose Dependency of Anti-Tumor Activity

1. Tumor Volume (50 ug, 100 ug, and 500 ug Doses)

70 female C57CL/6 mice, containing similar tumor volumes of approximately 50-51 mm³, following implantation of MC38 hPD-L1 tumor cells, were staged and divided into 5 treatment groups containing 14 mice each. Group 1 (isotype control) received 75 μg Fc only (SEQ ID NO: 1520); Groups 2, 3 and 4 received 50, 100, and 500 μg, respectively, CD80 variant E35D/M47L (SEQ ID NO: 208) fused to an inert human Fc (SEQ ID NO: 1520, or allotypes thereof) via a GSG₄S linker (SEQ ID NO: 1522); and Group 5 received 100 μg human anti-PD-L1 mAb (durvalumab), on days 8, 10, and 12. Tumor volumes were measured on days 7, 10, and 12. On day 13, 5 animals were sacrificed for analysis as described in the sections below. Tumor measurements resumed for the remaining 9 mice for each group on days 17, 20 and 27. On days 26, 28, and 31, the animals in Group 1 (Fc isotype control) received an intratumoral injection of 100 μg E35D/M47L CD80-IgV-Fc.

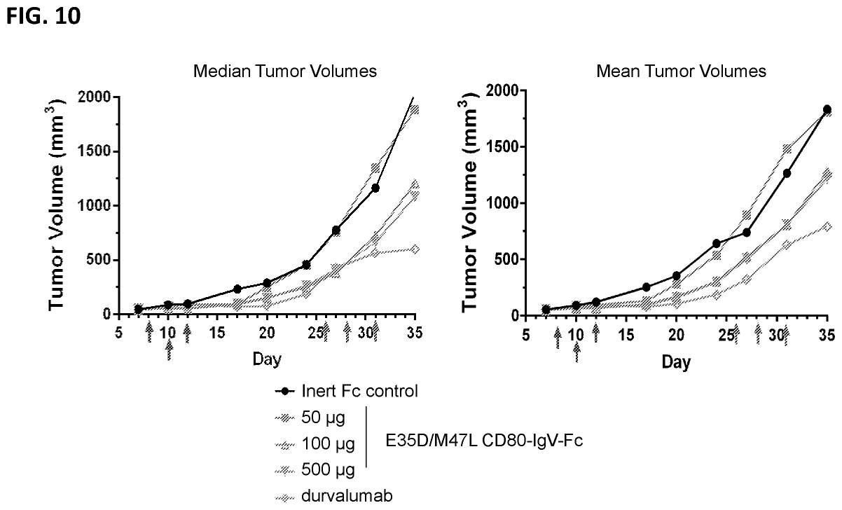

The median and mean tumor volumes are depicted in FIG. 10. As shown, a dose-dependent decrease in tumor volumes were observed in treated with CD80-IgV-Fc compared to the Fc control. In this study, the median tumor volume observed in mice treated with the 100 μg to 500 μg CD80-IgV-Fc was similar to mice treated with the anti-PD-L1 antibody control.

Cytokine Analysis

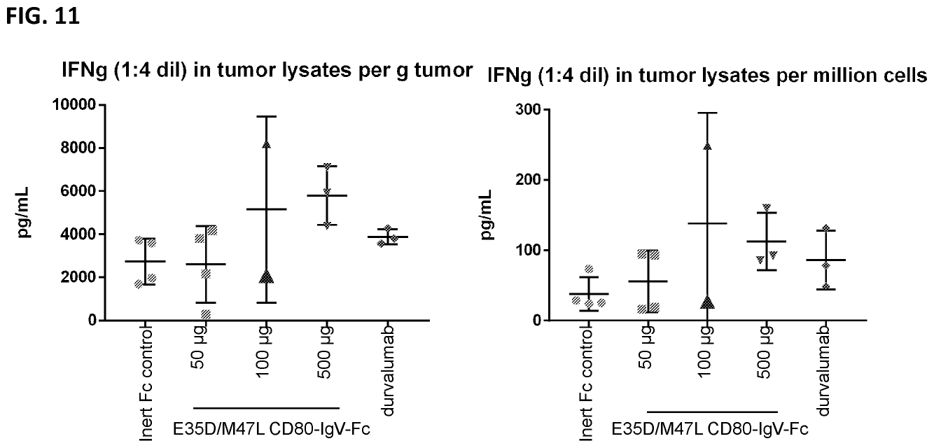

Following the enzymatic digestion of MC38 tumors, the lysate solution was centrifuged, and the supernatants collected and stored at −80° C. until ready for assay. The concentration of mouse IFNγ in each sample was then measured using a commercial ELISA kit (R&D Systems, Inc.) according to manufacturer's instructions, and concentrations were normalized based on either tumor weight or total cell number isolated from tumor. Results, set forth in FIG. 11, indicated that the highest dose (500 μg) of E35D/M47L CD80-IgV-Fc resulted in the highest concentrations of IFNγ in the tumor lysates, suggesting that the CD80-IgV-Fc is producing IFNγ as a result of its treatment, a mechanism that is known to promote anti-tumor immunity.

C. Anti-Tumor and Rechallenge Activity of CD80 Selected Variants

95 female C57BU6 mice were implanted with MC38 hPD-L1 tumor cells. The tumors were staged on Day 7, and 77 mice with similar tumor volumes of approximately 60 mm³ were divided into 7 treatment groups containing 11 mice each. Group 1 (Isotype control) received 75 μg inert Fc only (SEQ ID NO: 1520); Group 2 received 100 μg CD80 variant E35D/M47V/N48K/V68M/K89N IgV (SEQ ID NO: 465)-Fc (inert); Group 3 received 100 μg CD80 variant H18Y/A26E/E35D/M47L/V68M/A71G/D90G IgV (SEQ ID NO: 491)-Fc (inert); Group 4 received 100 pg CD80 variant E35D/D46V/M47U/V68M/L85Q/E88D IgV (SEQ ID NO: 495)-Fc (inert); Group 5 received 100 μg CD80 variant E35D/D46E/M47V/V68M/D90G/K93E IgV (SEQ ID NO: 499)-Fc (inert); Group 6 received 100 μg CD80 variant E35D/M47L (SEQ ID NO: 208)-Fc (inert); and Group 7 received 100 μg human anti-PD-L1 mAb (durvalumab), on days 7, 9 and 11. For the variant CD80-IgV-Fc molecules, the CD80IgV domains were fused to inert human Fc, such as set forth in SEQ ID NO: 1520, or allotypes thereof, via a GSG₄S linker (SEQ ID NO: 1522. Tumor volumes were measured on days 14, 17, 21, 24, 28, 31, and 37. Animals receiving the Fc isotype control were terminated by day 28 due to excess tumor burden.

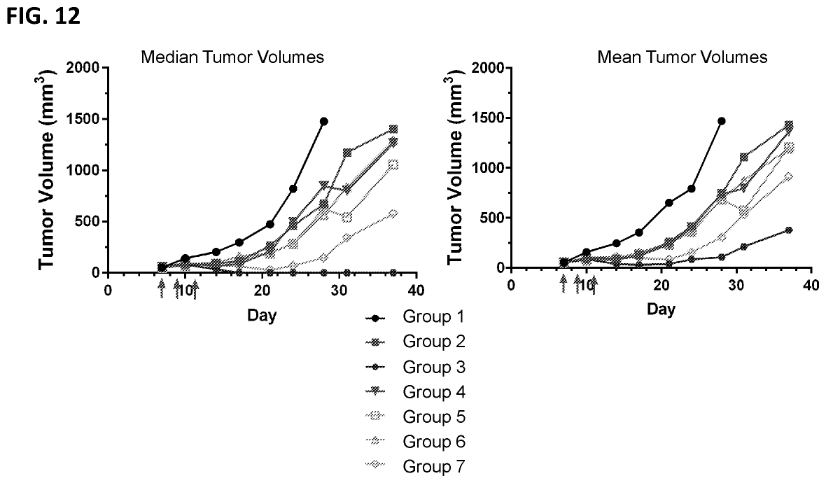

The median and mean tumor volumes are depicted in FIG. 12, which shows that all tested CD80-IgV-Fc molecules exhibited similar or, in some cases, substantially improved activity compared to the anti-PD-L1 control. Upon completion of the study, 8 mice from Group 3, 2 mice from Group 4, 1 mouse from Group 6, and 2 mice from Group 7 no longer had detectable tumors and were designated “tumor-free.”

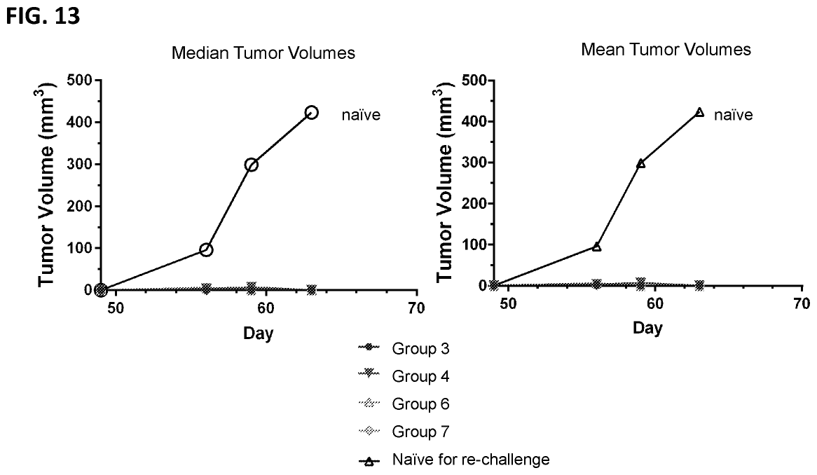

On day 49, tumor-free mice, from Groups 3, 4, 6, and 7, and 2 naïve C57CL/6 mice were re-challenged with an additional injection of hPD-L1 MC38 cells. Tumor volumes were measured on days 56, 59, and 63. The results are depicted in FIG. 13. Naïve mice exhibited rapid tumor growth, as expected. At day 59, 8/8 mice from Group 3, 1/2 mice from Group 4, 1/1 mouse from Group 6, and 2/2 mice from Group 7 were tumor-free, and by day 63, all mice in Group 3, Group 4, Group 6, and Group 7 were tumor-free. This result is consistent with an observation that the tested agents, including CD80-IgV-Fc molecules, were able to provide long-lasting, durable immunity, anti-tumor effects.

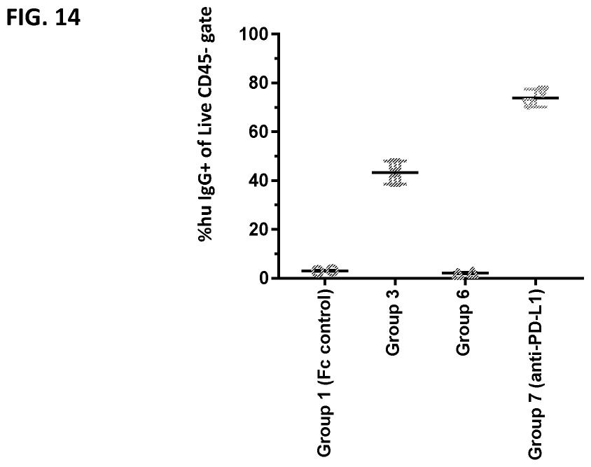

Tumors from mice sacrificed 3 days after the second dose were digested and live CD45-tumor cells were analyzed for the presence of bound inert Fc, CD80 variant-Fc, and anti-PD-L1 antibody by flow cytometry. The results for Groups 1, 3, 6 and 7 are provided in FIG. 14. Similar to the study described above, the results showed that the CD80-IgV-Fc molecules exhibited less binding to the tumor compared to the anti-PD-L1 antibody control. Despite this, superior activity by CD80-IgV-Fc, such as shown by mice treated with the exemplary CD80-IgV-Fc set forth in SEQ ID NO: 491 (H18Y/A26E/E35D/M47L/V68M/A71G/D90G), could be achieved consistent with the differentiating factor in activity being due to CD28 agonism (PD-L1-dependent CD28 costimulation) and/or CTLA-4 antagonism.

D. Anti-Tumor Activity of CD80 Variant and Anti-PD-L1 Antibody

75 animals were staged into 3 treatment groups 7 days after implantation with hPD-L1 MC38 tumor cells. Group 1 received 3 injections of 75 μg inert Fc (SEQ ID NO: 1520), Group 2 received 3 injections of 100 μg CD80 variant H18Y/A26E/E35D/M47L/V68M/A71G/D90G IgV (SEQ ID NO: 491)-Fc (inert), and Group 3 received 3 injections of 100 μg of human anti-PD-L1 mAb (durvalumab), with the injections taking place on Days 8, 10 and 12 after implantation. Tumor volumes were measured every 3-4 days, from Day 11 until Day 35. 3 days after the 1^{st} dose, 2^{nd} dose and 3^{rd} dose, 4 mice from each group were sacrificed for tumor and LN analyses, leaving 13 mice for tumor volume measurements throughout the study period.

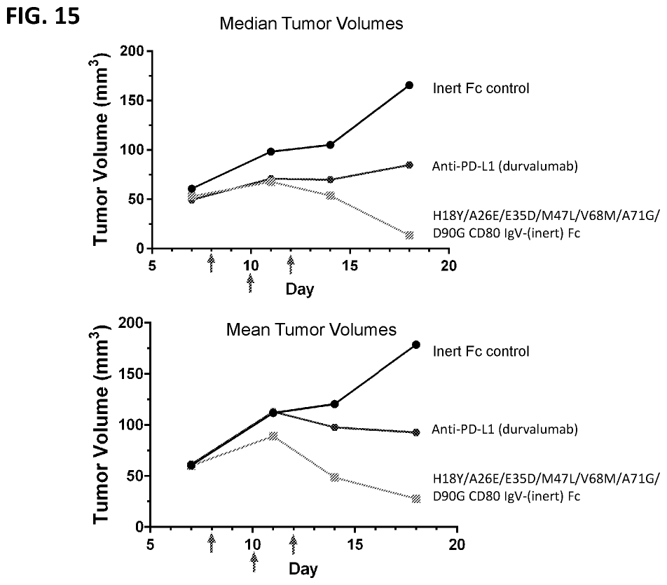

FIG. 15 shows a greater decrease in the median and mean tumor volumes of mice treated in this study with the exemplary CD80-IgV-Fc compared to the anti-PD-L1 control. On Day 18, 0/13 mice of Group 1 (Fc control-treated) were tumor-free, 6/13 mice of Group 2 (CD80 variant IgV-Fc-treated) were tumor-free, and 3/13 mice of Group 3 (durvalumab-treated) were tumor-free. At day 35, 1/13, 6/13, and 3/13 mice were tumor free in Groups 1, 2, and 3, respectively. Mice treated with the variant CD80-IgV-Fc exhibited tumors that on average were reduced in size compared to tumors of mice treated with anti-hPD-L1 antibody or the inert Fc control.

Tumor Cell Characterization

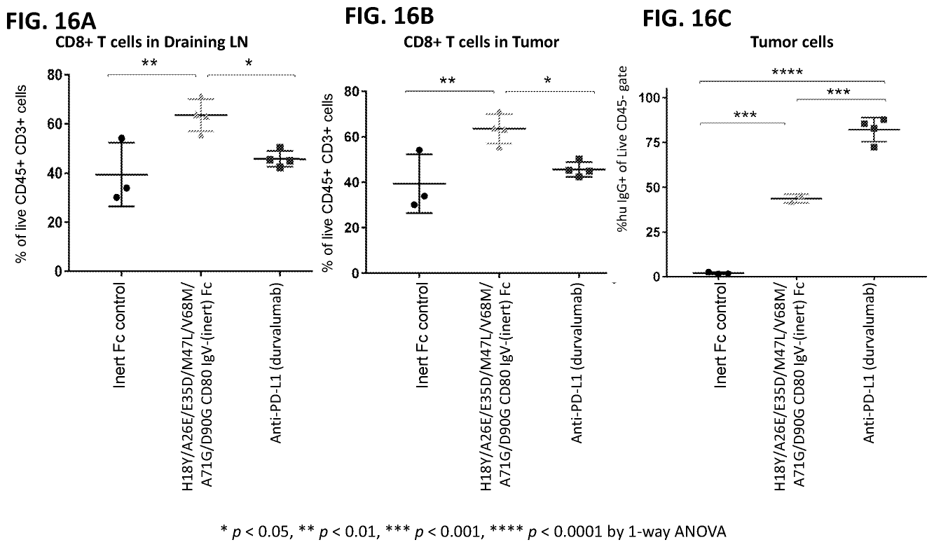

Three days following the 2nd dose of the Fc control, the CD80 variant IgV-Fc, and anti-PD-L1 antibody (durvalumab), tumors and draining lymph nodes (LN) were harvested from 34 mice from each treatment group. Tissues were processed to single cell suspensions (tumors were enzymatically digested as a part of the processing, whereas draining LN were not), and subjected to multi-color flow cytometric analysis of CD8+ T cells on the CD45+ cell subset (immune cells in either the LN or tumor), as well as % hIgG+ staining on the CD45− cell subset (tumor cells) to detect molecules (CD80-IgV-Fc or anti-PD-L1) bound to the tumor cells. The results are provided in FIG. 16A-C.

The percentages of CD8+ T cells were significantly greater (p<0.05 or p<0.01) in both the TIL and the LN for mice treated with H18Y/A26E/E35D/M47L/V68M/A71G/D90G CD80-IgV-Fc as compared to the Fc control or the anti-PD-L1 antibody treatments (FIGS. 16A (LN) and 16B (tumor). This indicates that H18Y/A26E/E35D/M47L/V68M/A71G/D90G CD80-IgV-Fc treatment can promote CD8+ T cell expansion in vivo, an important contributor to anti-tumor immunity. Furthermore, H18Y/A26E/E35D/M47L/V68M/A71G/D90G CD80-IgV-Fc was detected on the tumor (ex vivo via hIgG+ staining on CD45− cells) though at reduced levels as compared to those of the anti-PD-L1 antibody (FIG. 16C). Despite reduced presence of E35D/M47L CD80-Fc on the tumor, compared to anti-PD-L1 detected, the anti-tumor activity was superior for the CD80-Fc as compared to the anti-PD-L1 antibody (see section B1 above section). These results are consistent with an observation that the activity of CD80-IgV-Fc may not be only to PD-L1/PD-1 antagonism, but that the differentiating factor may relate to CD28 agonism (PD-L1-dependent CD28 costimulation) and/or CTLA-4 antagonism activities.

### Example 17

### Cytotoxicity to huPD-L1 Transduced MC38 Tumor Cells Compared to Anti-PD-L1 Antibody

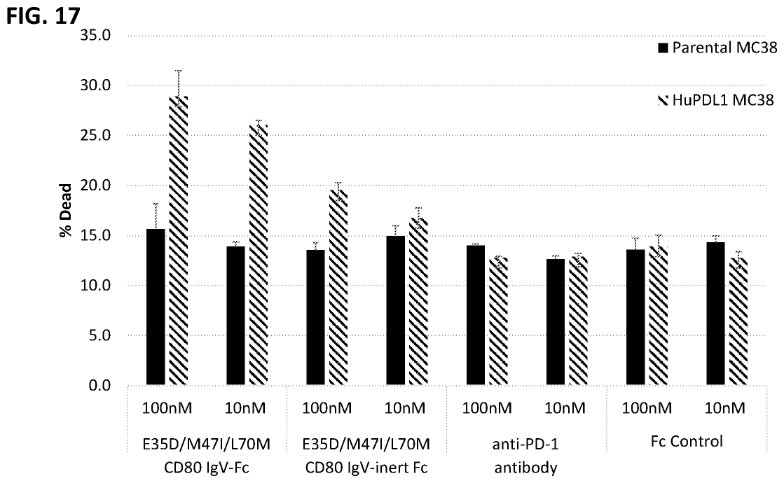

This Example describes the assessment of in vitro cytotoxicity of huPD-L1 transduced MC38 tumor cells. MC38 tumor cells, non-transduced or transduced with huPD-L1, were treated with Mitomycin-C and plated with human pan T cells labelled with CFSE at a 1:5 ratio. Variant CD80 IgV-Fc, containing E35D/M47I/L70M (SEQ ID NO: 125), with either WT IgG1 Fc or an inert Fc were added to MC38 tumor cells at 100 nM or 10 nM and cultured with cells for 72 hours. As a control, an exemplary anti-PD-1 antibody nivolumab or an Fc (inert) only control also were assessed. Cells were then harvested and stained with 7-AAD viability dye. After acquiring samples on a flow cytometer, the percentage of dead cells was calculated using Flowjo analysis by gating on 7-AAD+ cells in the CFSE− gate. As shown in FIG. 17, increased cytotoxicity against huPD-L1 transduced MC38 tumor cells, but not non-transduced MC38 parental cells, was observed by exemplary assessed variant CD80 IgV-Fc molecules. In this assay, cytotoxic activity was not observed in the presence of the control anti-PD-1 antibody, indicating that the variant CD80 IgV Fc molecules exhibit improved activity compared to the anti-PD-1 antibody control.

### Example 18

### CD80 Variant Binding to Primary Human T Cells and Monocytes

Binding of exemplary variant CD80-IgV Fc molecules to primary CD28+ human CD4 T cells and human PD-L1+ monocytes was assessed. The exemplary variant CD80 IgV-Fc molecules that were assessed contained E35D/M47V/N48K/V68M/K89N (SEQ ID NO: 465), H18Y/A26E/E35D/M47L/V68M/A71G/D90G (SEQ ID NO: 491), E35D/D46V/M47L/V68M/L85Q/E88D (SEQ ID NO: 495), and E35D/D46E/M47V/V68M/D90G/K93E (SEQ ID NO: 499).

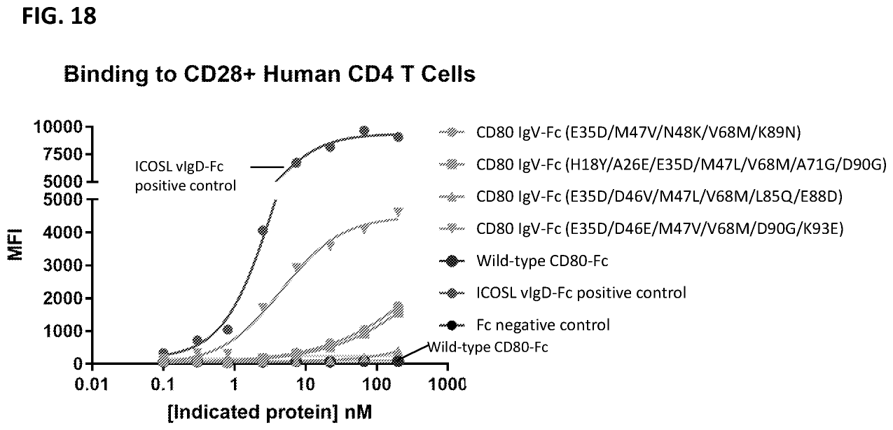

Unactivated human pan T cells were incubated with various concentrations of variant CD80 IgV-Fc and then were stained with anti-CD4, anti-CD8 and anti-human IgG to detect the Fc portion of the CD80 IgV-Fc. As a control, binding of wild-type CD80 IgV-Fc, an Fc only negative control, and a CD28-binding ICOSL vIgD-Fc also was assessed. Binding was assessed by flow cytometry and MFI was determined using Flowjo analysis software. As shown in FIG. 18, the tested variant CD80 IgV-Fc molecules demonstrated differential binding to primary human T cells, which, in some cases, was greater than wildtype CD80-IgV-Fc.

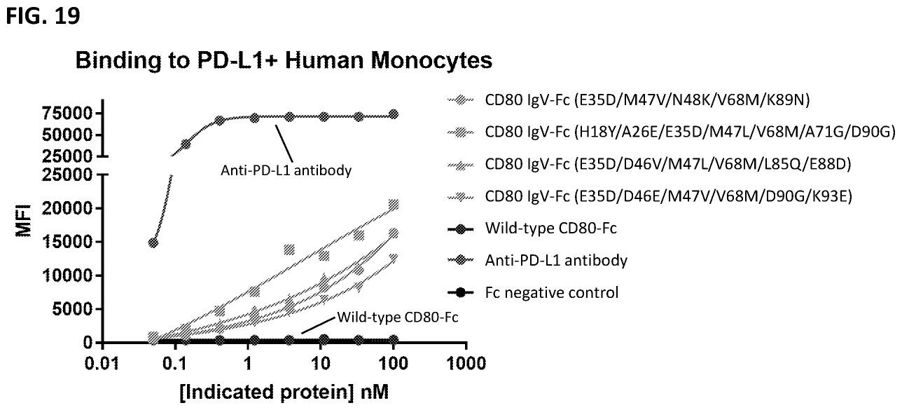

For binding to human monocyte-expressed PD-L1, human PBMC were plated overnight in the presence of anti-CD3 and anti-CD28. Cells were harvested the next day, incubated with various concentrations of variant CD80 IgV-Fc or an anti-PD-L1 antibody control (durvalumab), and then were stained with anti-CD14 to identify monocytes and anti-human IgG to detect the Fc portion of CD80 IgV molecules. Binding was assessed by flow cytometry and MFI was determined using Flowjo analysis software. As shown in FIG. 19, all tested variant CD80 IgV-Fc molecules demonstrated substantially greater binding to primary human monocytes than wild-type CD80 IgV-Fc.

### Example 19

### Variant CD80 IgV-Fc Antagonism of PD-L1 Mediated PD-1 SHP2 Recruitment

This Example describes a Jurkat/PD-1/SHP2 Signaling Assay to assess the effect of the variant CD80 IgV-Fc molecules to antagonize the recruitment of the cytoplasmic protein tryrosine phosphatase SHP-2 to PD-1 by blocking PD-L1/PD-1 interaction. In an exemplary assay, a Jurkat cell line containing a recombinant β-galactosidase (β-gal) fragment Enzyme Donor (ED) tagged PD-1 receptor and an Enzyme Acceptor (EA) tagged SHP-2 domain were used (e.g. DiscoverX, USA; cat. 93-1106C19). In the assay, SHP-2 recruitment to PD-1 results in the EA and ED being in close proximity to allow complementation of the two enzyme fragments forming a functional beta-Gal enzyme that hydrolyzes a substrate to generate a chemiluminescent signal.

K562/OKT3/PD-L1 aAPC were pre-incubated with various concentrations of exemplary variant CD80 IgV-Fc (inert) for 30 minutes. The exemplary variant CD80 IgV-Fc molecules that were assessed contained H18Y/A26E/E35D/M47L/V68M/A71G/D90G (SEQ ID NO: 491), E35D/M47V/N48K/V68M/K89N (SEQ ID NO: 465), E35D/D46V/M47L/V68M/L85Q/E88D (SEQ ID NO: 495), and E35D/D46E/M47V/V68M/D90G/K93E (SEQ ID NO: 499). As a control, wild-type CD80 IgV-Fc (inert), an anti-PD-L1 antibody, and an Fc (inert) only control were also assessed. Jurkat/PD-1/SHP2 cells (DiscoverX Pathhunter Enzyme Complementation Fragment Recruitment line) were added and cells were incubated for 2 hours. The substrate for beta-Gal (DiscoverX Bioassay Detection reagent) was added to the wells, incubated for 1 hour at room temperature in the dark, and the luciferase was measured on a microplate reader (BioTek Cytation).

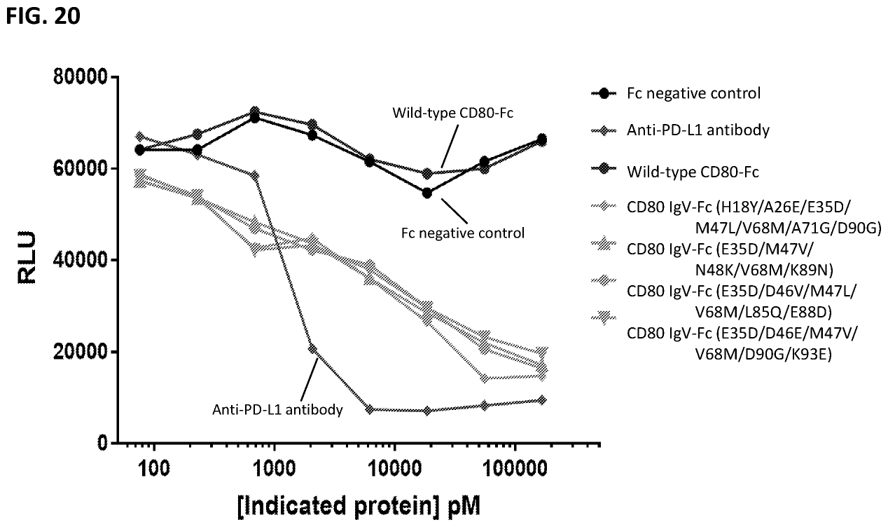

As shown in FIG. 20, the exemplary variant CD80 IgV-Fc molecules decreased luciferase activity, consistent with an observation that the variant CD80 IgV-Fc molecules exhibited potent activity to antagonize PD-L1 mediated PD-1 SHP2 recruitment. Potent antagonist activity also was observed by the anti-PD-L1 positive control, but the wild-type CD80 IgV-Fc molecule did not exhibit PD-1/PD-L1 antagonist activity as evidenced by no decrease in luciferase signal detected in the presence of a wild-type CD80 IgV-Fc molecule.

### Example 20

### CD80 Variant Antagonism of B7/CTLA-4 Binding

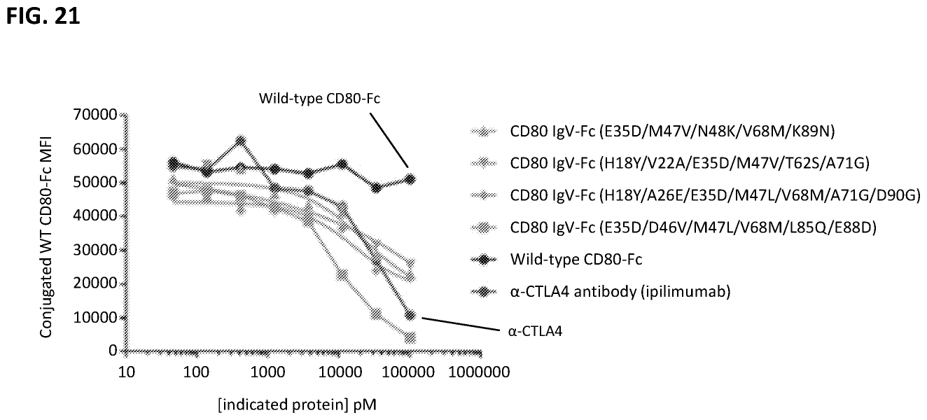

To assess the ability of CD80 vIgD-Fc to antagonize the interaction of CTLA-4 and B7 binding, CHO cells, stably expressing surface human CTLA-4 were plated with a titration of E35D/M47V/N48K/V68M/K89N (SEQ ID NO: 465), H18Y/V22A/E35D/M47V/T62S/A71G (SEQ ID NO: 490), H18Y/A26E/E35D/M47L/V68M/A71G/D90G (SEQ ID NO: 491) E35D/D46V/M47L/V68M/L85Q/E88D (SEQ ID NO: 495), or wild-type CD80 vIgD-Fc, or an anti-CTLA-4 antibody (ipilimumab) as a positive control. After washing, cells were incubated with 25 nM fluorochrome-conjugated wild-type CD80-Fc. Bound fluorescent competitor protein was detected and measured by flow cytometry. As shown in FIG. 21, all CD80 vIgD-Fc variants, but not wild-type CD80-Fc, antagonized the binding of CD80 to CTLA-4.

### Example 21

### Assessment of Combination of CD80 Variant Molecule and Anti-PD-1 Antibody in the huPD-L1/B16-F10 Melanoma Model

This Example describes the assessment of anti-tumor activity of exemplary tested variant CD80 IgV-Fc (inert) (variant CD80 IgV containing amino acid substitutions H18Y/A26E/E35D/M47L/V68M/A71G/D90G; SEQ ID NO: 491). This variant is an exemplary variant identified to have increased binding affinity for PD-L1 compared to wild-type CD80 and activity to block PD-L1 and CTLA-4 and to provide PD-L1-dependent T cell activation via CD28 costimulatory receptor. To test the anti-tumor activity of the exemplary variant, it was evaluated alone or in combination with an anti-mouse PD-1 monoclonal antibody (clone RMP1-14, rat IgG2a) in mice bearing human PD-L1 (huPD-L1)-expressing B16-F10 tumors, which is a syngeneic mouse melanoma model. This model is an aggressive and, in many cases, a treatment-resistant model.

The B16-F10 cell line was transduced with huPD-L1 to ensure target expression on the tumor by the variant CD80 IgV-Fc. Subconfluent cells (˜80% confluent) were harvested on the day of implantation (study day 0). The cells were washed twice and brought to a final concentration of 5×10⁶ cells/mL in DPBS. Female C57BL/6NJ mice (Jackson Labs, USA) were implanted subcutaneously with approximately 0.5×10⁶ huPD-L1/B16-F10 cells. For injections, 0.1 mL of cells (0.5×10⁶ cells) were injected subcutaneously (SC) per mouse in the right mid-flank region. The B16-F10 cells at time of implant were evaluated to confirm expression of huPD-L1 by flow cytometry. Mice were staged on Day 6 and randomized to groups with similar mean tumor volumes (43 mm³).

On day 6, mice were randomized into four groups of 12 mice each, with each group having a similar mean tumor volume (42.8 mm³). The tested molecules were delivered through intraperitoneal (IP) injection, with a total of 3 doses delivered via IP injection, on days 6, 8 and 11 as outlined in Table E28.

TABLE E28
Treatment Descriptions

| | | | | Dose | Dose | Route |
|---|---|---|---|---|---|---|
| | | | | Volume | Schedule | of |
| Group | # of | | Dose | (mL/ | (D = | Deliv- |
| # | Mice | Test Article(s) | Level | kg) | day) | ery |
| 1 | 12 | Fc control | 75 μg | 5 | D6, D8, D11 | IP |
| 2 | 12 | Variant CD80 | 100 μg | 5 | D6, D8, D11 | IP |
| | | IgV-Fc | | | | |
| | | (H18Y/A26E/ | | | | |
| | | E35D/M47L/ | | | | |
| | | V68M/A71G/ | | | | |
| | | D90G; | | | | |
| | | SEQ ID | | | | |
| | | NO: 491) | | | | |
| 3 | 12 | Anti-mouse | 100 μg | 5 | D6, D8, D11 | IP |
| | | PD-1 mAb | | | | |
| 4 | 12 | Variant CD80 | 100 μg | 5 | D6, D8, D11 | IP |
| | | IgV-Fc | | | | |
| | | (H18Y/A26E/ | | | | |
| | | E35D/ | | | | |
| | | M47L/V68M/ | | | | |
| | | A71G/D90G; | | | | |
| | | SEQ ID | | | | |
| | | NO: 491) | | | | |
| | | Anti-mouse | 100 μg | 5 | D6, D8, D11 | IP |
| | | PD-1 mAb | | | | |

Tumors were measured with electronic calipers two-dimensionally twice weekly, beginning on day 6 post-tumor cell implant. Tumor volume was calculated as length×(width×2)×0.5, with the length being the longer of the two measurements. Tumor growth inhibition (TGI) values were obtained as measures of anti-tumor activity calculated using the following formula: [(mean or median Fc control tumor size −mean or median treated tumor size) divided by mean or median Fc control tumor size]×100. Calculations for the mean and median were determined on the last day in which at least 70% of mice were alive on study (day 18 post-tumor cell implant).

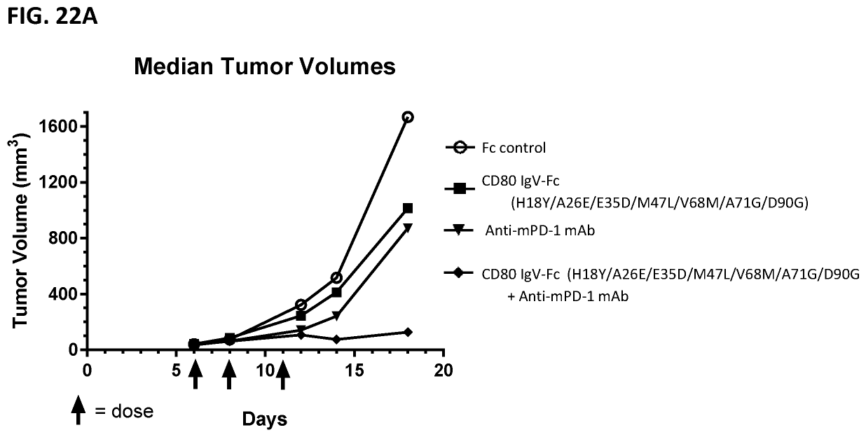

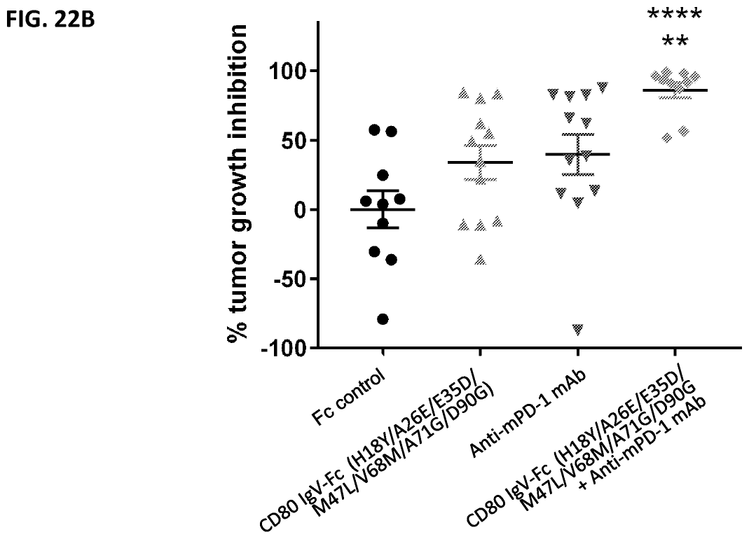

As shown in FIG. 22A, the combination of variant CD80 IgV-Fc (H18Y/A26E/E35D/M47L/V68M/A71G/D90G) and mPD-1 mAb significantly reduced tumor growth (median tumor volumes) over time compared to groups treated with Fc control, variant CD80 IgV-Fc (H18Y/A26E/E35D/M47L/V68M/A71G/D90G) alone or anti-mouse PD-1 mAb alone (p<0.05; 2-way repeated measures ANOVA).

A percent mean and median tumor growth inhibition (TGI) among individual mice treated were also determined based on tumor volumes from the last day in which at least 70% of mice from each group were alive on study (day 18), using the following formula: [(mean or median Fc control tumor size −mean or median test article treated tumor size) divided by Fc control mean or median tumor size]×100). The anti-tumor activity of the combination as measured by TGI shown in Table E29 and FIG. 22B is consistent with the finding that the combination of variant CD80 IgV-Fc and mPD-1 mAb significantly reduced tumor growth compared to control groups (Kruskal-Wallis test: ** p<0.01 versus the variant CD80-IgV-Fc and anti-mPD-1 mAb groups; **** p<0.0001 versus the Fc control group). The results showed that the exemplary variant CD80 IgV-Fc (H18Y/A26E/E35D/M47L/V68M/A71G/D90G) was particularly effective (92% tumor growth inhibition) in improving the antitumor activity of anti-PD-1 mAb in mice bearing huPD-L1+B16-F10 tumors, a tumor that is known to be poorly immunogenic and treatment-recalcitrant.

TABLE E29
Summary of Anti-Tumor Growth Activity Measured by TGI

| | | | Dose | | Mean | Median |
|---|---|---|---|---|---|---|
| Group | | Dose | Schedule | | TGI | TGI |
| # | Test Article | Level | (days) | Route | (D18) | (D18) |
| 1 | Fc control | 75 μg | D6, D8, D11 | IP | n/a | n/a |
| 2 | Variant | 100 μg | D6, D8, D11 | IP | 33.9 | 39.2 |
| | CD80 IgV-Fc | | | | | |
| | (H18Y/A26E/ | | | | | |
| | E35D/M47L/ | | | | | |
| | V68M/A71G/ | | | | | |
| | D90G; | | | | | |
| | SEQ ID NO: 491) | | | | | |
| 3 | Anti-mouse | 100 μg | D6, D8, D11 | IP | 39.8 | 47.8 |
| | PD-1 mAb | | | | | |
| 4 | Variant | 100 μg | D6, D8, D11 | IP | 86.1 | 92.4 |
| | CD80 IgV-Fc | | | | | |
| | (H18Y/A26E/ | | | | | |
| | E35D/M47L/ | | | | | |
| | V68M/A71G/ | | | | | |
| | D90G; | | | | | |
| | SEQ ID NO: 491) | | | | | |
| | Anti-mouse | 100 μg | D6, D8, D11 | IP | | |
| | PD-1 mAb | | | | | |

These results demonstrate substantial improvements in anti-tumor activity of a combination therapy including anti-PD-1 and an exemplary provided variant CD80-Fc polypeptides, such as variant CD80 IgV-Fc (inert), including those that exhibits increased binding affinity to PD-L1.

### Example 22

### Assessment of T Cell Response with Combination of CD80 Variant Molecule and Anti-PD-1 Antibody

A cytomegalovirus (CMV) antigen-specific functional assay was used to assess the effect of combination of an anti-PD-1 antibody (e.g. nivolumab) and an exemplary tested variant CD80 IgV-Fc (inert) (H18Y/A26E/E35D/M47L/V68M/A71G/D90G; SEQ ID NO: 491) on T cell responses.

Peripheral blood mononuclear cells (PBMC) obtained from CMV seropositive donor were thawed and CMV lysate added at 1 μg/mL to 250,000/well PBMC. The tested exemplary variant CD80 IgV-Fc or wild-type CD80 ECD-Fc was added at various concentrations in the presence or absence of 50 nM concentration of anti-PD-1 antibody (nivolumab). In addition, the anti-PD-1 antibody alone was also tested. An Fc only molecule was also tested as control. Supernatant was collected 48 hours after incubation to assay IL-2 by ELISA.

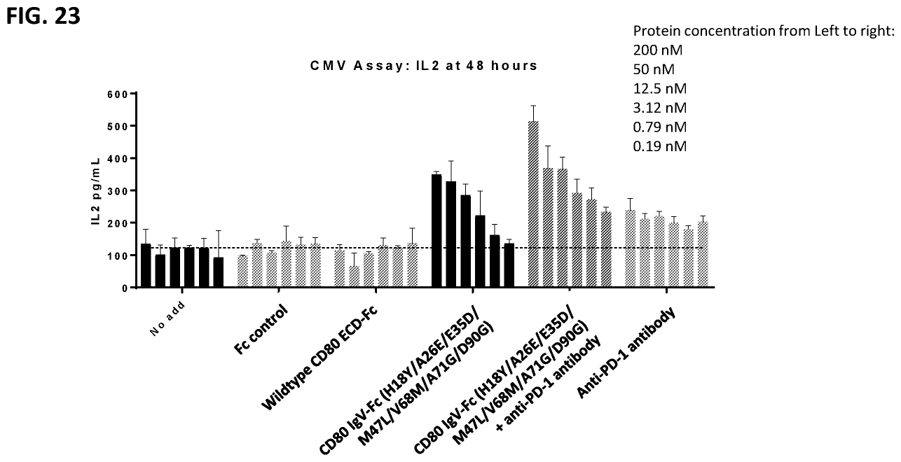

As shown in FIG. 23, the tested variant CD80 IgV-Fc molecule showed augmentation of IL-2 production compared to the Fc only control. A dose-dependent increase in IL-2 production also was observed in the presence of increasing concentrations of variant CD80 IgV-Fc. Furthermore, the combination of the tested variant CD80 IgV-Fc molecule and anti-PD-1 antibody showed greater increase in IL-2 production compared to levels of IL-2 production in the presence of either molecule alone.

### Example 23

### Assessment of Dosing and Delivery of CD80 Variant Molecules

This Example describes the assessment of anti-tumor activity of exemplary tested variant CD80 IgV-Fc (inert) (H18Y/A26E/E35D/M47L/V68M/A71G/D90G; SEQ ID NO: 491) in a syngeneic mouse tumor model of huPD-L1-expressing MC38 tumors delivered by either intraperitoneal (IP) dose or by intratumoral (IT) injections.

A. Tumor Model

The MC38 cell line was transduced with human PD-L1 (huPD-L1) Subconfluent cells (˜80% confluent) were harvested on the day of implantation (study day 0), washed twice and brought to a final concentration of 15×10⁶ cells/mL in DPBS. Female C57BL/6NJ mice (Jackson Labs, USA) were implanted subcutaneously with approximately 1.5×10⁶ cells (0.1 mL) in the right mid-flank region.

On day 7, mice were randomized into six groups, with each group having a similar mean tumor volume (46.6 mm³).

B. Tumor Growth Assessment

The exemplary tested variant CD80 IgV-Fc (H18Y/A26E/E35D/M47L/V68M/A71G/D90G; SEQ ID NO: 491) was delivered to groups of tumor-bearing mice once via IP injection on day 8 post-cell implantation at 100, 500 and 1500 μg. The 500 μg dose was also administered to a group of mice as a split dose (three IP injections of 167 μg each, given on days 8, 11 and 14 post-cell implant). An additional group of mice received the variant CD80 IgV-Fc via IT delivery on days 8, 11 and 14 at 100 μg each day. Groups treated with the test molecule were compared to a group of mice that human Fc control by one IP injection on day 8. Treatment groups are summarized in Table E30.

TABLE E30
Summary of Treatment Groups

| | | | | | | | Mice (N) |
|---|---|---|---|---|---|---|---|
| | | | | Dose | | | for Ex |
| | | | | Vol- | Dose | Route | Vivo |
| | # of | | | ume | Schedule | of | Tumor |
| Group | Mice | | Dose | (mL/ | (D = | Deli- | Analysis |
| # | (total) | Test Article | Level | kg) | day) | very | (D11) |
| 1 | 14 | Fc control | 75 μg | 5 | D8 × 1 | IP | 5 |
| 2 | 14 | Variant | 100 μg | 5 | D8 × 1 | IP | 5 |
| 3 | 14 | CD80 IgV- | 500 μg | 5 | D8 × 1 | IP | 5 |
| 4 | 14 | Fc | 1500 μg | 5 | D8 × 1 | IP | 5 |
| 5 | 9 | (H18Y/A26 | 167 μg | 5 | D8, 11, 14 | IP | 0 |
| 6 | 14 | E/E35D/ | 100 μg | 1 | D8, 11, 14 | IT | 5 |
| | | M47L/ | | | | | |
| | | V68M/ | | | | | |
| | | A71G/ | | | | | |
| | | D90G) | | | | | |

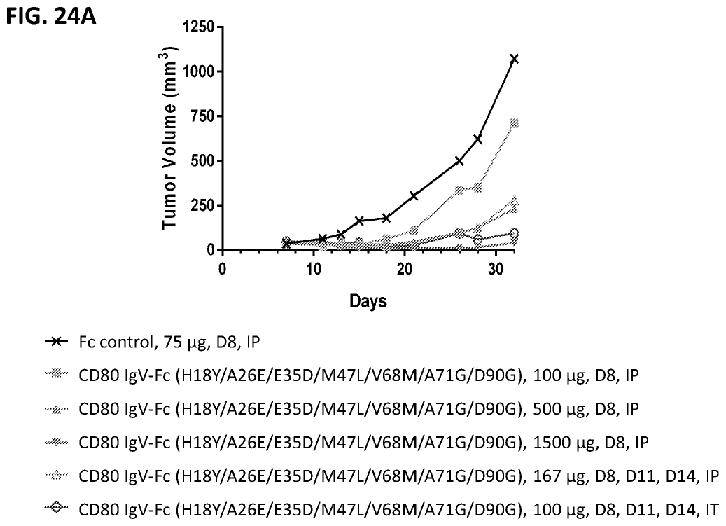

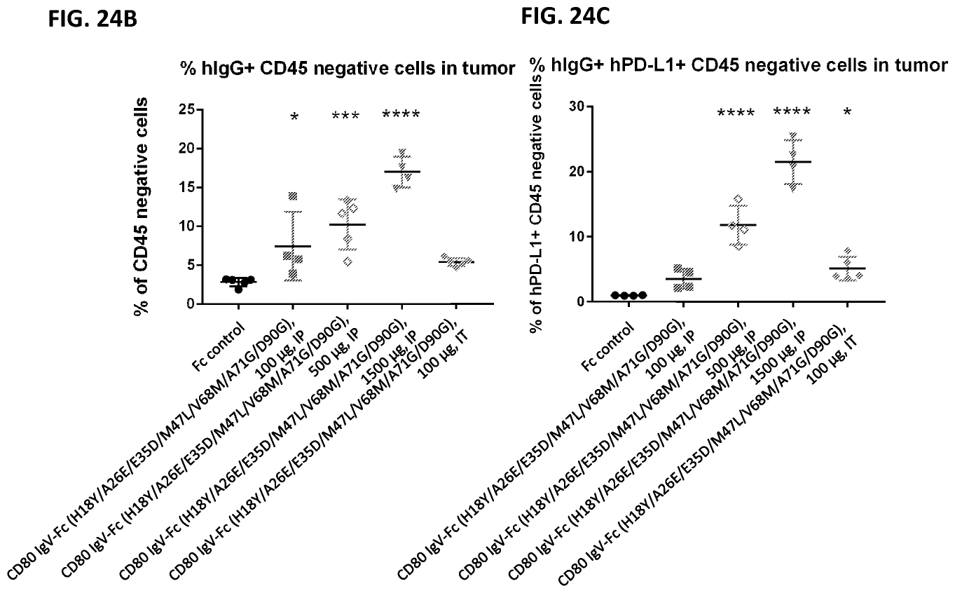

Tumor volume was measured as described in Example 21 to assess anti-tumor activity. Median tumor growth curves among individual treated mice over time were plotted. As shown in FIG. 24A, mice treated with the exemplary tested variant CD80 IgV-Fc by a single IP injection were characterized by a dose-dependent increase in anti-tumor activity compared to Fc control treatment. Each of the groups that received 100 (as one or three IP injections), 500 μg (as one IP injections) or 1500 μg of variant CD80 IgV-Fc by IP injection demonstrated substantial reduction in tumor volume compared to control mice. Dosing by IT injection also resulted in substantial anti-tumor growth activity as compared to the Fc control-treated group.

C. Tumor Analysis

On day 11 post-tumor cell implant and prior to dosing of test articles, five mice from each of Group 1-4 and Group 6 were sacrificed, and the tumors were harvested, weighed and processed for ex vivo tumor analysis. The MC38 cells at time of implant and tumors harvested from a subset of mice on day 11 by enzymatic digestion were evaluated for expression of huPD-L1 or human IgG (to detect Fc of cell-bound test molecules) by flow cytometry. The percent positive and median fluorescent intensity (MFI) of huPD-L1 and huIgG were quantified on the CD45-negative subset of tumor cells.

The percent of cells positive for PD-L1 among CD45-negative cells from day 11 harvested cells was similar among all treatment groups.

For the groups of mice treated IP with variant CD80 IgV-Fc, the day 11 tumor cells showed a significant and dose-dependent increase in the percent of cells detected with bound test article using anti-huIgG among CD45-negative cell subset (FIG. 24B) or among PD-L1+CD45− cell subset (FIG. 24C). This observation is consistent with dose-dependent tumor localization of the tested molecule, and demonstrates an increased tumor exposure with higher doses of the variant CD80 IgV-Fc. The mice that received 100 μg variant CD80 IgV-Fc by IT delivery had a comparable percentage of detected therapeutic huIgG staining at this time point (reflecting only the first dose of the variant CD80 IgV-Fc delivered IT) that was similar to the same dose level administered by IP injection.

Tumors at day 11 were also evaluated for CD8+ T cells and for antigen-specific CD8+ T cells within the CD3+ cell population. p15E is an MHC class I restricted T cell antigen expressed in the MC38 tumor cell lines derived from C57BL/6 mice. A mouse MHC class I p15E tetramer labeled with PE (MBL International Corp.) was used to detect the p15E-MHC class I restricted T cell receptor.

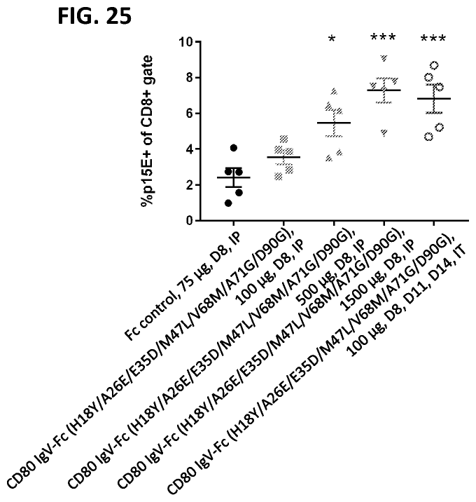

The percentage of p15e tetramer+CD8+ T cells among total cells in the tumors was determined. Cells from tumors from mice treated with either 500 or 1500 μg variant CD80 IgV-Fc (IP) or 100 μg variant CD80 IgV-Fc (IT) had a significantly greater percent of p15E-specific CD8+ T cells as compared to Fc control-treated mice (FIG. 25). This result shows that higher doses of variant CD80 IgV-Fc delivered IP or doses administered IT result in increased percentages of antigen-specific CD8+ T cells in the tumors.

### Example 24

### Generation of Multivalent Variant CD80 IgSF Domain Fusion Proteins and Binding Assessment

This Example describes generation of variant CD80 IgSF domain fusion proteins containing at least two affinity modified IgV domains from identified variant CD80 polypeptides. Specifically, two units of exemplary variant CD80 IgV with H18Y/A26E/E35D/M47L/V68M/A71G/D90G (SEQ ID NO: 491) were linked together and fused to an Fc in various configurations. Tetravalent and hexavalent molecules were generated.

A. Generation of Multivalent Proteins

Multivalent variant CD80 IgSF domain fusion proteins were generated in various configurations as follows. Multivalent variant CD80 IgSF domain fusion proteins were expressed ad purified substantially as described in Example 5. In the generated multivalent proteins, the variant CD80 IgV variants were variously linked to the N- or C-terminus of a human IgG1 Fc region via a GSGGGGS (SEQ ID NO:1522) or 3× GGGGS (SEQ ID NO: 1504) peptide linker. In this study, constructs were generated using either an effectorless human IgG1 Fc region (inert Fc) or a human IgG1 Fc region capable of mediating effector activity (effector Fc).

The inert Fc region used in generated constructs had the sequence set forth in SEQ ID NO: 1518 and contained the mutation C220S, L234A, L235E, G237A, by EU numbering (the mutations corresponded to C5S, L19A, L20E, G22A, with reference to wild-type human IgG1 Fc set forth in SEQ ID NO: 1502). In some cases, the Fc contained removal of the C-terminal lysine, K447del by EU numbering (corresponding to deletion of position 232, with reference to wild-type or unmodified Fc set forth in SEQ ID NO: 1502).

The effector Fc had the sequence set forth in SEQ ID NO: 1527 and contained the mutation C220S, E356D and M358L, by EU numbering (the mutations corresponded to C5S, E141D, and M143L, which further contained removal of the C-terminal lysine, K447del by EU numbering (corresponding to deletion of position 232) with reference to wild-type human IgG1 Fc (set forth in SEQ ID NO: 1502). Other Fc regions also are suitable for generation of multivalent molecules.

SEQ ID NO: 1518
EPKSSDKTHTCPPCPAPEAEGAPSVFLFPPKPKD
TLMISRTPEVTCVVVDVSHEDPEVKFNWYVDGVE
VHNAKTKPREEQYNSTYRVVSVLTVLHQDWLNGK
EYKCKVSNKALPAPIEKTISKAKGQPREPQVYTL
PPSRDELTKNQVSLTCLVKGFYPSDIAVEWESNG
QPENNYKTTPPVLDSDGSFFLYSKLTVDKSRWQQ
GNVFSCSVMHEALHNHYTQKSLSLSPG
SEQ ID NO: 1527
EPKSSDKTHTCPPCPAPELLGGPSVFLFPPKPKD
TLMISRTPEVTCVVVDVSHEDPEVKFNWYVDGVE
VHNAKTKPREEQYNSTYRVVSVLTVLHQDWLNGK
EYKCKVSNKALPAPIEKTISKAKGQPREPQVYTL
PPSRDELTKNQVSLTCLVKGFYPSDIAVEWESNG
QPENNYKTTPPVLDSDGSFFLYSKLTVDKSRWQQ
GNVFSCSVMHEALHNHYTQKSLSLSPG

Nucleic acid molecules encoding the multivalent constructs also contained residues encoding the exemplary signal peptide MGSTAILALLLAVLQGVSA (set forth in SEQ ID NO: 276). Expression constructs encoding Fc fusion proteins of interest were transiently expressed in Expi293. For each multivalent protein, the encoding nucleic acid molecule was designed to produce proteins in various configurations with sequences in the order shown:

Multivalent variant CD80 IgSF domain
fusion protein 1 (SEQ ID NO: 1529):
CD80 variant IgV (SEQ ID NO: 491)-
GSGGGGS (SEQ ID NO: 1522)-
Fc (SEQ ID NO: 1518)-3x GGGGS
(SEQ ID NO: 1504)-CD80 variant
IgV (SEQ ID NO: 491)
Multivalent variant CD80 IgSF domain
fusion protein 2 (SEQ ID NO: 1531):
CD80 variant IgV (SEQ ID NO: 491)-
GSGGGGS (SEQ ID NO: 1522)-
Fc (SEQ ID NO: 1527)-
3x GGGGS (SEQ ID NO: 1504)-
CD80 variant IgV (SEQ ID NO: 491)
Multivalent variant CD80 IgSF domain
fusion protein 3 (SEQ ID NO: 1533):
CD80 variant IgV (SEQ ID NO: 491)-
3x GGGGS (SEQ ID NO: 1504)-
CD80 variant IgV (SEQ ID NO: 491)-
GSGGGGS (SEQ ID NO: 1522)-
Fc (SEQ ID NO: 1518)
Multivalent variant CD80 IgSF domain
fusion protein 4 (SEQ ID NO: 1535):
CD80 variant IgV (SEQ ID NO: 491)-
3x GGGGS (SEQ ID NO: 1504)-
CD80 variant IgV (SEQ ID NO: 491)-
GSGGGGS (SEQ ID NO: 1522)-
Fc (SEQ ID NO: 1527)

B. Binding Assessment

Binding assays were carried out to assess the specificity and affinity of multivalent proteins to cell-expressed CTLA-4, CD28, and PD-L1 counter structures. The multivalent variant CD80 IgSF domain fusion proteins were tested for binding, substantially as described in Example 7 except that 11.1 nM of each variant CD80 Fc-fusion molecules were added to cells engineered to express the indicated cognate counter structure ligand (i.e., CTLA-4, PD-L1, or CD28). The ratio of the MFI of the multivalent variant CD80 IgV-Fc, compared to the binding of the unmodified CD80-ECD-Fc fusion molecule (R&D Systems, USA) not containing the amino acid substitution(s), to the same cell-expressed counter structure ligand is shown in Table E31. As shown, the multivalent variant CD80 IgSF domain fusion proteins exhibited increased binding for one or more of the counter structures.

TABLE E31
Binding to cells stably expressing CD28, CTLA4 or PD-L1

| | Protein | Flow Binding to Jurkats (CD28) and | | | | | | | | |
|---|---|---|---|---|---|---|---|---|---|---|
| | SEQ | CHO cells stably expressing CTLA4 or PD-L1 | | | | | | | | |
| | ID NO | CTLA4 | | | | CD28 | | PD-L1 | | |
| | (DNA | | Fold | | | Fold | | | Fold | |
| | SEQ | | change | | | change | | | change | |
| | ID | MFI at | to WT | EC50 | MFI at | to WT | EC50 | MFI at | to WT | EC50 |
| Description | NO) | 11.1 nM | CD80 | (nM) | 11.1 nM | CD80 | (nM) | 11.1 nM | CD80 | (nM) |
| Multivalent | 1529 | 10486 | 0.6 | 1.1 | 2736 | 19.7 | 0.2 | 14349 | 166.8 | 2.1 |
| protein 1: | (1528) | | | | | | | | | |
| Variant CD80 | | | | | | | | | | |
| IgV-GSG4S- | | | | | | | | | | |
| Fc-3xG4S- | | | | | | | | | | |
| variant CD80 | | | | | | | | | | |
| IgV | | | | | | | | | | |
| Multivalent | 1531 | 10564 | 0.6 | 1.1 | 2636 | 19.0 | 0.3 | 12757 | 148.3 | 3.0 |
| protein 2: | (1530) | | | | | | | | | |
| Variant CD80 | | | | | | | | | | |
| IgV-GSG4S- | | | | | | | | | | |
| Fc-3xG4S- | | | | | | | | | | |
| variant CD80 | | | | | | | | | | |
| IgV | | | | | | | | | | |
| Multivalent | 1533 | 10993 | 0.7 | 5.0 | 2736 | 19.7 | 0.4 | 14822 | 172.3 | 3.0 |
| protein 3: | (1532) | | | | | | | | | |
| Variant CD80 | | | | | | | | | | |
| IgV-3xG4S- | | | | | | | | | | |
| variant CD80 | | | | | | | | | | |
| IgV-GSG4S- | | | | | | | | | | |
| Fc | | | | | | | | | | |
| Multivalent | 1535 | 12479 | 0.8 | 1.9 | 2702 | 19.4 | 0.5 | 15819 | 183.9 | 0.9 |
| protein 4: | (1534) | | | | | | | | | |
| Variant CD80 | | | | | | | | | | |
| IgV-3xG4S- | | | | | | | | | | |
| variant CD80 | | | | | | | | | | |
| IgV-GSG4S- | | | | | | | | | | |
| Fc | | | | | | | | | | |
| CD80 IgV-Fc | 491 | 16243 | 1.0 | 1.3 | 2630 | 18.9 | 6.5 | 19622 | 228.2 | 3.6 |
| WT CD80 | | 16610 | 1.0 | 2.2 | 139 | 1.0 | 164.4 | 86 | 1.0 | 31.2 |
| ECD-Fc | | | | | | | | | | |
| Fc control | | 106 | 0.0 | 0.0 | 26 | 0.2 | 0.0 | 158 | 1.8 | 0.0 |

C. Blocking PD-L1/PD-1 Interaction

The multivalent variant CD80 IgSF domain fusion proteins were assessed to test for blocking of the PD-L1/PD-1 and CTLA-4/CD80 interaction performed substantially as described in Example 15. CHO/OKT3/PD-L1 and CHO/CTLA-4 cells were counted and plated at 100,000 cells/well and then incubated with fluorescently conjugated PD-1-Fc and CD80-Fc. Exemplary multivalent variant CD80 IgSF domain fusion proteins were incubated with the cells for 30 minutes. As a control, an Fc only molecule, anti-PD-L1 antibody, anti-CTA-4 antibody and a bivalent variant CD80 IgV-Fc were also assessed. Cells were washed and incubated with 100 nM fluorescently conjugated PD-1-Fc and CD80-Fc competitor for 30 minutes. Cells were then washed and samples were acquired on a flow cytometer and MFIs of the fluorescently labeled molecules were determined.

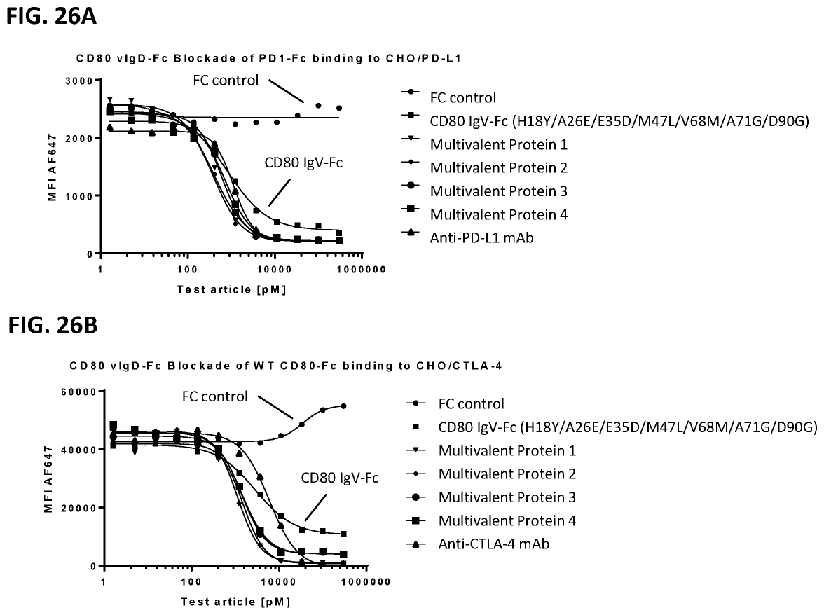

As shown in FIGS. 26A and 26B, the exemplary multivalent variant CD80 IgV-Fc molecules tested were shown to antagonize or block binding of PD-1 to PD-L1 and CD80 to CTLA-4. Improved antagonist activity to block PD1/PD-L1 and CD80/CTLA-4 was observed compared to the bivalent variant CD80 IgV-Fc.

D. Cytomegalovirus (CMV) Antigen Specific T Cell Response

The multivalent variant CD80 IgSF domain fusion proteins were assessed to test for IL-2 production in the CMV assay substantially as described in Example 22. Exemplary multivalent variant CD80 IgSF domain fusion proteins generated as described above were tested at various concentrations from 100000 pM to 46 pM

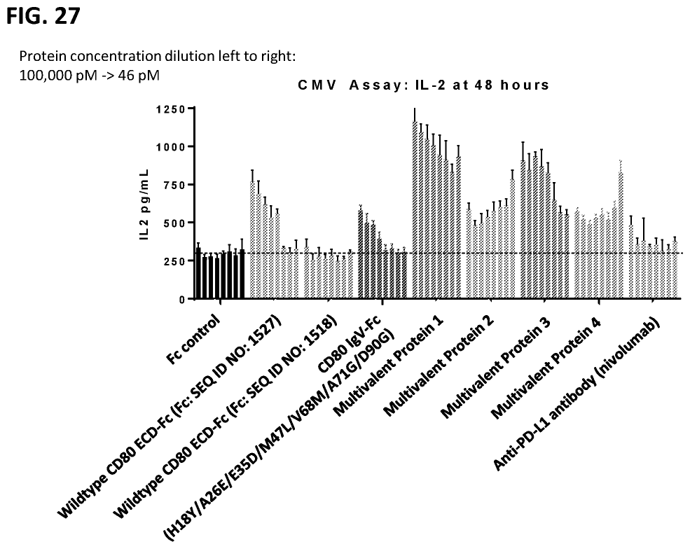

As shown in FIG. 27, the tested multivalent molecules showed a greater increase in IL-2 production compared to levels of IL-2 production in the presence of molecules containing a wildtype CD80 ECD-Fc molecule or the variant CD80 IgV-Fc molecule in a bivalent format. The effectorless multivalent molecules containing the Fc set forth in SEQ ID NO: 1518 (inert) showed increased IL-2 productions compared to corresponding molecules on the effector Fc backbone (SEQ ID NO: 1527). Particularly at higher concentrations, lower levels of IL-2 production were observed with effector Fc constructs compared to inert Fc constructs. Without wishing to be bound by theory, the differences in activity with the different Fc constructs may be due to higher exposure or affinity for PD-L1 vs. FcR in this assay, thereby resulting in a greater PD-L1-dependent CD28 agonism than FcR-dependent CD28 agonism in this assay.

### Example 25

### Generation of Additional Variant CD80 IgV Domains and Assessment of Binding Activity

Constructs were generated based on a wildtype human CD80 amino acid sequence of the extracellular domain (ECD) set forth in SEQ ID NO: 2 (corresponding to residues 35-242 as set forth in UniProt Accession No. P33681) as follows:

VIHVTKEVKEVATLSCGHNVSVEELAQTRIYWQKE
KKMVLTMMSGDMNIWPEYKNRTIFDITNNLSIVIL
ALRPSDEGTYECVVLKYEKDAFKREHLAEVTLSV
KADFPTPSISDFEIPTSNIRRIICSTSGGFPEPH
LSWLENGEELNAINTTVSQDPETELYAVSSKLDF
NMTTNHSFMCLIKYGHLRVNQTFNWNTTKQEHFP
DN

For these variants, yeast displayed targeted or random CD80 libraries were selected against each of CD28, CTLA-4 and PD-L1 separately. The CD80 variants were generated and expressed as Fc fusion proteins essentially as described in Examples 2-5.

A. Binding to Cell-Expressed Counter Structures

This Example describes Fc-fusion binding studies to show specificity and affinity of CD80 domain variant immunomodulatory proteins for cognate binding partners.

To produce cells expressing cognate binding partners, full-length mammalian surface expression constructs for each of human CD28, CTLA-4 and PD-L1, were designed in pcDNA3.1 expression vector (Life Technologies) and sourced from Genscript, USA. Binding studies were carried out using the Expi293F transient transfection system (Life Technologies, USA). The number of cells needed for the experiment was determined, and the appropriate 30 ml scale of transfection was performed using the manufacturer's suggested protocol. For each CD28, CTLA-4, PD-L1, or mock 30 ml transfection, 75 million Expi293F cells were incubated with 30 μg expression construct DNA and 1.5 ml diluted ExpiFectamine 293 reagent for 48 hours, at which point cells were harvested for staining.

For staining by flow cytometry, 200,000 cells of appropriate transient transfection or negative control were plated in 96-well round bottom plates. Cells were spun down and resuspended in staining buffer (PBS (phosphate buffered saline), 1% BSA (bovine serum albumin), and 0.1% sodium azide) for 20 minutes to block non-specific binding. Afterwards, cells were centrifuged again and resuspended in staining buffer containing 100 nM to 1 nM variant immunomodulatory protein, depending on the experiment of each candidate variant CD80 Fc protein in 50 μl. Primary staining was performed on ice for 45 minutes, before washing cells in staining buffer twice. PE-conjugated anti-human Fc (Jackson ImmunoResearch, USA) was diluted 1:150 in 50 μl staining buffer and added to cells and incubated another 30 minutes on ice. Secondary antibody was washed out twice, cells were fixed in 4% formaldehyde/PBS, and samples were analyzed on FACScan flow cytometer (Becton Dickinson, USA).

Mean Fluorescence Intensity (MFI) was calculated for each transfectant and negative parental line with Cell Quest Pro software (Becton Dickinson, USA).

B. Bioactivity Characterization

This Example further describes Fc-fusion variant protein bioactivity characterization in human primary T cell in vitro assays.

1. Mixed Lymphocyte Reaction (MLR)

Soluble rCD80.Fc bioactivity was tested in a human Mixed Lymphocyte Reaction (MLR). Human primary dendritic cells (DC) were generated by culturing monocytes isolated from PBMC (BenTech Bio, USA) in vitro for 7 days with 500U/ml rIL-4 (R&D Systems, USA) and 250 U/ml rGM-CSF (R&D Systems, USA) in Ex-Vivo 15 media (Lonza, Switzerland). 10,000 matured DC and 100,000 purified allogeneic CD4+ T cells (BenTech Bio, USA) were co-cultured with variant CD80 Fc fusion proteins and controls in 96 well round bottom plates in 200 μl final volume of Ex-Vivo 15 media. On day 5, IFN-gamma secretion in culture supernatants was analyzed using the Human IFN-gamma Duoset ELISA kit (R&D Systems, USA). Optical density was measured by VMax ELISA Microplate Reader (Molecular Devices, USA) and quantitated against titrated rIFN-gamma standard included in the IFN-gamma Duo-set kit (R&D Systems, USA).

2. Anti-CD3 Coimmobilization Assay

Costimulatory bioactivity of CD80 fusion variants was determined in anti-CD3 coimmobilization assays. 1 nM or 4 nM mouse anti-human CD3 (OKT3, Biolegends, USA) was diluted in PBS with 1 nM to 80 nM rCD80.Fc variant proteins. This mixture was added to tissue culture treated flat bottom 96-well plates (Corning, USA) overnight to facilitate adherence of the stimulatory proteins to the wells of the plate. The next day, unbound protein was washed off the plates and 100,000 purified human pan T cells (BenTech Bio, US) or human T cell clone BC3 (Astarte Biologics, USA) were added to each well in a final volume of 200 μl of Ex-Vivo 15 media (Lonza, Switzerland). Cells were cultured 3 days before harvesting culture supernatants and measuring human IFN-gamma levels with Duoset ELISA kit (R&D Systems, USA) as described above.

C. Results

Results for the binding and activity studies for exemplary tested variants are shown in Tables E32, E33, and E34. In particular, Table E32 indicates exemplary IgSF domain amino acid substitutions (replacements) in the ECD of CD80 selected in the screen for affinity-maturation against the respective cognate structure CD28. Table E33 indicates exemplary IgSF domain amino acid substitutions (replacements) in the ECD of CD80 selected in the screen for affinity-maturation against the respective cognate structure PD-L1. For the Tables, the exemplary amino acid substitutions are designated by amino acid position number corresponding to the respective reference unmodified ECD sequence.

Also shown is the binding activity as measured by the Mean Fluorescence Intensity (MFI) value for binding of each variant Fc-fusion molecule to cells engineered to express the cognate counter structure ligand and the ratio of the MFI compared to the binding of the corresponding unmodified ECD-Fc fusion molecule not containing the amino acid substitution(s) to the same cell-expressed counter structure ligand. The functional activity of the variant Fc-fusion molecules to modulate the activity of T cells also is shown based on the calculated levels of IFN-gamma in culture supernatants (pg/ml) generated either i) with the indicated variant ECD-Fc fusion molecule coimmobilized with anti-CD3 or ii) with the indicated variant ECD-Fc fusion molecule in an MLR assay. Tables E32-34 also depict the ratio of IFN-gamma produced by each variant ECD-Fc compared to the corresponding unmodified ECD-Fc in both functional assays.

As shown, the selections resulted in the identification of a number of CD80 IgSF domain variants that were affinity-modified to exhibit increased binding for at least one, and in some cases more than one, cognate counter structure ligand. In addition, the results showed that affinity modification of the variant molecules also exhibited improved activities to both increase and decrease immunological activity depending on the format of the molecule. For example, coimmobilization of the ligand likely provides a multivalent interaction with the cell to cluster or increase the avidity to favor agonist activity and increase T cell activation compared to the unmodified (e.g. wildtype) ECD-Fc molecule not containing the amino acid replacement(s). However, when the molecule is provided as a bivalent Fc molecule in solution, the same IgSF domain variants exhibited an antagonist activity to decrease T cell activation compared to the unmodified (e.g. wildtype) ECD-Fv molecule not containing the amino acid replacement(s).

TABLE E32
Variant CD80 selected against CD28. Molecule sequences, binding data, and
costimulatory bioactivity data.

| | | | | | Coimmobilization | |
|---|---|---|---|---|---|---|
| | | | | | with anti CD3 | MLR |
| | | Binding | | | IFN- | IFN-gamma |
| | | CD28 | CTLA- | PD-L1 | gamma | levels |
| | SEQ | MFI | 4 MFI | MFI | pg/ml | pg/ml |
| | ID NO | (parental | (parental | (parental | (parental | (parental |
| CD80 mutation(s) | (ECD) | ratio) | ratio) | ratio) | ratio) | ratio) |
| L70Q/A91G/N144D | 1383 | 125 | 283 | 6 | 93 | 716 |
| | | (1.31) | (1.36) | (0.08) | (1.12) | (0.83) |
| L70Q/A91G/T130A | 1448 | 96 | 234 | 7 | 99 | 752 |
| | | (1.01) | (1.13) | (0.10) | (1.19) | (0.87) |
| L70Q/A91G/I118A/ | 1384 | 123 | 226 | 7 | 86 | 741 |
| T120S/T130A/K169E | | (1.29) | (1.09) | (0.10) | (1.03) | (0.86) |
| V4M/L70Q/A91G/I118V/ | 1385 | 89 | 263 | 6 | 139 | 991 |
| T120S/T130A/K169E | | (0.94) | (1.26) | (0.09) | (1.67) | (1.14) |
| L70Q/A91G/I118V/T120S/ | 1386 | 106 | 263 | 6 | 104 | 741 |
| T130A/K169E | | (1.12) | (1.26) | (0.09) | (1.25) | (0.86) |
| V20L/L70Q/A91S/I118V/ | 1388 | 105 | 200 | 9 | 195 | 710 |
| T120S/T130A | | (1.11) | (0.96) | (0.13) | (2.34) | (0.82) |
| S44P/L70Q/A91G/T130A | 1453 | 88 | 134 | 5 | 142 | 854 |
| | | (0.92) | (0.64) | (0.07) | (1.71) | (0.99) |
| L70Q/A91G/E117G/I118V/ | 1389 | 120 | 193 | 6 | 98 | 736 |
| T120S/T130A | | (1.27) | (0.93) | (0.08) | (1.05) | (0.85) |
| A91G/T120S/I118V/T130A | 1390 | 84 | 231 | 44 | 276 | 714 |
| | | (0.89) | (1.11) | (0.62) | (3.33) | (0.82) |
| L70R/A91G/I118V/T120S/ | 1391 | 125 | 227 | 6 | 105 | 702 |
| T130A/T199S | | (1.32) | (1.09) | (0.09) | (1.26) | (0.81) |
| L70Q/E81A/A91G/I118V/T120S/ | 1392 | 140 | 185 | 18 | 98 | 772 |
| I127T/T130A | | (1.48) | (0.89) | (0.25) | (1.18) | (0.89) |
| L70Q/Y87N/A91G/T130A | 1458 | 108 | 181 | 6 | 136 | 769 |
| | | (1.13) | (0.87) | (0.08) | (1.63) | (0.89) |
| T28S/L70Q/A91G/ | 1393 | 32 | 65 | 6 | 120 | 834 |
| E95K/I118V/T120S/I126V/ | | (0.34) | (0.31) | (0.08) | (1.44) | (0.96) |
| T130A/K169E | | | | | | |
| N63S/L70Q/A91G/ | 1394 | 124 | 165 | 6 | 116 | 705 |
| S114T/I118V/T120S/T130A | | (1.30) | (0.79) | (0.08) | (1.39) | (0.81) |
| K36E/I67T/L70Q/A91G/ | 1395 | 8 | 21 | 5 | 53 | 852 |
| I118V/T1205/T130A/N152T | | (0.09) | (0.10) | (0.08) | (0.63) | (0.98) |
| E52G/L70Q/A91G/D107N/I118V/ | 1396 | 113 | 245 | 6 | 94 | 874 |
| T1205/T130A/K169E | | (1.19) | (1.18) | (0.08) | (1.13) | (1.01) |
| K37E/F595/L70Q/A91G/T120S/ | 1397 | 20 | 74 | 6 | 109 | 863 |
| T130A | | (0.21) | (0.36) | (0.08) | (1.31) | (1.00) |
| A91G/S103P | 1464 | 39 | 56 | 9 | 124 | 670 |
| | | (0.41) | (0.27) | (0.13) | (1.49) | (0.77) |
| K89E/T130A | 1465 | 90 | 148 | 75 | 204 | 761 |
| | | (0.95) | (0.71) | (1.07) | (2.45) | (0.88) |
| A91G | 1466 | 96 | 200 | 85 | 220 | 877 |
| | | (1.01) | (0.96) | (1.21) | (2.65) | (1.01) |
| D60V/A91G/I118V/T120S/ | 1398 | 111 | 222 | 12 | 120 | 744 |
| T130A/K169E | | (1.17) | (1.07) | (0.18) | (1.44) | (0.86) |
| K54M/L70Q/A91G/Y164H | 1399 | 68 | 131 | 5 | 152 | 685 |
| | | (0.71) | (0.63) | (0.08) | (1.83) | (0.79) |
| M38T/L70Q/E77G/A91G/ | 1400 | 61 | 102 | 5 | 119 | 796 |
| I118V/T120S/T130A/N152T | | (0.64) | (0.49) | (0.07) | (1.43) | (0.92) |
| R29H/E52G/L70R/ | 1470 | 100 | 119 | 5 | 200 | 740 |
| E88G/A91G/T130A | | (1.05) | (0.57) | (0.08) | (2.41) | (0.85) |
| Y31H/T41G/M43L/L70Q/A91G/ | 1401 | 85 | 85 | 6 | 288 | 782 |
| I118V/T120S//I126V/T130A | | (0.89) | (0.41) | (0.08) | (3.47) | (0.90) |
| V68A/T110A | 1472 | 103 | 233 | 48 | 163 | 861 |
| | | (1.08) | (1.12) | (0.68) | (1.96) | (0.99) |
| L65H/D90G/T110A/F116L | 1402 | 33 | 121 | 11 | 129 | 758 |
| | | (0.35) | (0.58) | (0.15) | (1.55) | (0.88) |
| R29H/E52G/D90N/I118V/T120S/ | 1403 | 66 | 141 | 11 | 124 | 800 |
| T130A | | (0.69) | (0.68) | (0.15) | (1.49) | (0.92) |
| A91G/L102S | 1475 | 6 | 6 | 5 | 75 | 698 |
| | | (0.06) | (0.03) | (0.08) | (0.90) | (0.81) |
| I67T/L70Q/A91G/I118V/T120S | 1405 | 98 | 160 | 5 | 1751 | 794 |
| | | (1.03) | (0.77) | (0.08) | (21.1) | (0.92) |
| L70Q/A91G/T110A/ | 1406 | 8 | 14 | 5 | 77 | 656 |
| I118V/T120S/T130A | | (0.09) | (0.07) | (0.07) | (0.93) | (0.76) |
| M38V/T41D/M43I/ | 1407 | 5 | 8 | 8 | 82 | 671 |
| W50G/D76G/V83A/ | | (0.06) | (0.04) | (0.11) | (0.99) | (0.78) |
| K89E/I118V/T120S/I126V/T130A | | | | | | |
| V22A/L70Q/S121P | 1479 | 5 | 7 | 5 | 105 | 976 |
| | | (0.06) | (0.04) | (0.07) | (1.27) | (1.13) |
| A12V/S15F/Y31H/M38L/ | 1408 | 6 | 6 | 5 | 104 | 711 |
| T41G/M43L/D90N/T130A/P137L/ | | (0.06) | (0.03) | (0.08) | (1.25) | (0.82) |
| N149D/N152T | | | | | | |
| I67F/L70R/E88G/A91G/ | 1409 | 5 | 6 | 6 | 62 | 1003 |
| I118V/T120S/T130A | | (0.05) | (0.03) | (0.08) | (0.74) | (1.16) |
| E24G/L25P/L70Q/A91G/I118V/ | 1410 | 26 | 38 | 8 | 101 | 969 |
| T120S/N152T | | (0.27) | (0.18) | (0.11) | (1.21) | (1.12) |
| A91G/F92L/F108L/1118V/T120S | 1411 | 50 | 128 | 16 | 59 | 665 |
| | | (0.53) | (0.61) | (0.11) | (0.71) | (0.77) |
| WT CD80 | 2 | 95 | 208 | 70 | 83 | 866 |
| | | (1.00) | (1.00) | (1.00) | (1.00) | (1.00) |

TABLE E33
Variant CD80 selected against PD-L1. Molecule sequences, binding data, and
costimulatory bioactivity data.

| | | | | | Coimmobilization | |
|---|---|---|---|---|---|---|
| | | | | | with | MLR |
| | | Binding | | | anti-CD3 | IFN-gamma |
| | | CD28 | CTLA-4 | PD-L1 | IFN-gamma | levels |
| | SEQ ID | MFI | MFI | MFI | pg/ml | pg/ml |
| | NO | (parental | (parental | (parental | (parental | (parental |
| CD80 mutation(s) | (ECD) | ratio) | ratio) | ratio) | ratio) | ratio) |
| R29D/Y31L/Q33H/ | 1484 | 1071 | 1089 | 37245 | 387 | 5028 |
| K36G/M38I/T41A/ | | (0.08) | (0.02) | (2.09) | (0.76) | (0.26) |
| M43R/M47T/E81V/ | | | | | | |
| L85R/K89N/A91T/ | | | | | | |
| F92P/K93V/R94L/ | | | | | | |
| I118T/N149S | | | | | | |
| R29D/Y31L/Q33H/ | 1485 | 1065 | 956 | 30713 | 400 | 7943 |
| K36G/M38I/T41A/ | | (0.08) | (0.02) | (1.72) | (0.79) | (0.41) |
| M43R/M47T/E81V/ | | | | | | |
| L85R/K89N/A91T/ | | | | | | |
| F92P/K93V/R94L/ | | | | | | |
| N144S/N149S | | | | | | |
| R29D/Y31L/Q33H/ | 1486 | 926 | 954 | 47072 | 464 | 17387 |
| K36G/M38I/T41A/ | | (0.07) | (0.02) | (2.64) | (0.91) | (0.91) |
| M42T/M43R/M47T/ | | | | | | |
| E81V/L85R/K89N/ | | | | | | |
| A91T/F92P/K93V/ | | | | | | |
| R94L/L148S/N149S | | | | | | |
| E24G/R29D/Y31L/ | 1487 | 1074 | 1022 | 1121 | 406 | 13146 |
| Q33H/K36G/M38I/ | | (0.08) | (0.02) | (0.06) | (0.80) | (0.69) |
| T41A/M43R/M47T/ | | | | | | |
| F59L/E81V/L85R/ | | | | | | |
| K89N/A91T/F92P/ | | | | | | |
| K93V/R94L/H96R/ | | | | | | |
| N149S/C182S | | | | | | |
| R29D/Y31L/Q33H/ | 1488 | 1018 | 974 | 25434 | 405 | 24029 |
| K36G/M38I/T41A/ | | (0.08) | (0.02) | (1.43) | (0.80) | (1.25) |
| M43R/M47T/E81V/ | | | | | | |
| L85R/K89N/A91T/ | | | | | | |
| F92P/K93V/R94L/N149S | | | | | | |
| R29V/M43Q/E81R/ | 1489 | 1029 | 996 | 1575 | 342 | 11695 |
| L85I/K89R/D90L/ | | (0.08) | (0.02) | (0.09) | (0.67) | (0.61) |
| A91E/F92N/K93Q/R94G | | | | | | |
| T41I/A91G | 1490 | 17890 | 50624 | 12562 | 433 | 26052 |
| | | (1.35) | (1.01) | (0.70) | (0.85) | (1.36) |
| E88D/K89R/D90K/A91G/ | 1412 | 41687 | 49429 | 20140 | 773 | 6345 |
| F92Y/K93R/N122S/ | | (3.15) | (0.99) | (1.13) | (1.52) | (0.33) |
| N177S | | | | | | |
| E88D/K89R/D90K/A91G/ | 1494 | 51663 | 72214 | 26405 | 1125 | 9356 |
| F92Y/K93R | | (3.91) | (1.44) | (1.48) | (2.21) | (0.49) |
| K36G/K37Q/M38I/ | 1414 | 1298 | 1271 | 3126 | 507 | 3095 |
| L40M/F59L/E81V/L85R/ | | (0.10) | (0.03) | (0.18) | (1.00) | (0.16) |
| K89N/A91T/F92P/ | | | | | | |
| K93V/R94L/E99G/ | | | | | | |
| T130A/N149S | | | | | | |
| E88D/K89R/D90K/ | 1494 | 31535 | 50868 | 29077 | 944 | 5922 |
| A91G/F92Y/K93R | | (2.38) | (1.02) | (1.63) | (1.85) | (0.31) |
| K36G/K37Q/M38I/L40M | 1495 | 1170 | 1405 | 959 | 427 | 811 |
| | | (0.09) | (0.03) | (0.05) | (0.84) | (0.04) |
| K36G/L40M | 1412 | 29766 | 58889 | 20143 | 699 | 30558 |
| | | (2.25) | (1.18) | (1.13) | (1.37) | (1.59) |
| WTCD80 | 2 | 13224 | 50101 | 17846 | 509 | 19211 |
| | | (1.00) | (1.00) | (1.00) | (1.00) | (1.00) |

TABLE E34
CD80 variants selected against CTLA-4 or PD-L1. Molecule sequences, binding data,
and costimulatory bioactivity data.

| | | PD-Ll | Anti-CD3 IFN- |
|---|---|---|---|
| CD28 | CTLA-4 | tfxn | gamma |

| | SEQ ID | tfxn MFI | tfxn MFI | MFI | Coimmobilization |
|---|---|---|---|---|---|
| | NO | (parental | (parental | (parental | Assay pg/ml |
| CD80 mutation(s) | (ECD) | ratio) | ratio) | ratio) | (parental ratio) |
| R29D, Y31L, Q33H, K36G, M38I, | 1415 | 3536 | 5731 | 173405 | 109 (0.24) |
| T41A, M43R, M47T, E81V, L85R, | | (0.08) | (0.01) | (0.08) | |
| K89N, A91T, F92P, K93V, R94L, | | | | | |
| I118V, T120S, I127T, T130A, | | | | | |
| K169E | | | | | |
| R29D, Y31L, Q33H, K36G, M38I, | 1416 | 4962 | 2027 | 626341 | 162 (0.36) |
| T41A, M43R, M47T, L70Q, E81V, | | (0.11) | (0.01) | (0.11) | |
| L85R, K89N, A91T, F92P, K93V, | | | | | |
| R94L, I118V, T120S, I127T, | | | | | |
| T130A, | | | | | |
| H18L, R29D, Y31L, Q33H, K36G, | 1417 | 3489 | 2521 | 215826 | 206 (0.46) |
| M38I, T41A, M43R, M47T, E81V, | | (0.08) | (0.01) | (0.08) | |
| L85R, K89N, A91T, F92P, K93V, | | | | | |
| R94L, I118V, T120S, I127T, | | | | | |
| T130A, K169E | | | | | |
| R29D, Y31L, Q33H, K36G, M38I, | 1418 | 2736 | 2493 | 157897 | 141 (0.31) |
| T41A, M43R, M47T, E81V, L85R, | | (0.06) | (0.01) | (0.06) | |
| K89N, A91T, F92P, K93V, R94L, | | | | | |
| I118V, T120S, T130A, K169E, | | | | | |
| M174T | | | | | |
| R29D, Y31L, Q33H, K36G, M38I, | 1419 | 2393 | 2663 | 137062 | 230 (0.51) |
| T41A, M43R, M47T, N48D, F59L, | | (0.05) | (0.01) | (0.05) | |
| E81V, L85R, K89N, A91T, F92P, | | | | | |
| K93V, R94L, I118V, T120S, I127T, | | | | | |
| T130A, H188D | | | | | |
| H18R, R29D, Y31L, Q33H, K36G, | 1420 | 3023 | 2303 | 158977 | 305 (0.68) |
| K37E, M38I, T41A, M43R, M47T, | | (0.07) | (0.01) | (0.07) | |
| L70Q, E81V, L85R, K89N, A91T, | | | | | |
| F92P, K93V, R94L, I118V, T120S, | | | | | |
| T130A, K169E, H188D | | | | | |
| R29D, Y31L, Q33H, K36G, M38I, | 1421 | 2135 | 2816 | 374117 | 291 (0.65) |
| T41A, M43R, M47T, L70Q, E81V, | | (0.05) | (0.01) | (0.05) | |
| L85R, K89N, A91T, F92P, K93V, | | | | | |
| R94L, I118V, T120S, I127T, | | | | | |
| T130A, E143G, K169E, M174V, | | | | | |
| H188D | | | | | |
| R29D, Y31L, Q33H, K36G, M38I, | 1415 | 2157 | 2819 | 114963 | 197 (0.44) |
| T41A, M43R, M47T, E81V, L85R, | | (0.05) | (0.01) | (0.05) | |
| K89N, A91T, F92P, K93V, R94L, | | | | | |
| I118V, T120S, I127T, T130A, | | | | | |
| K169E | | | | | |
| R29D, Y31L, Q33H, K36G, M38I, | 1416 | 2126 | 2377 | 530029 | 135 (0.30) |
| T41A, M43R, M47T, L70Q, E81V, | | (0.05) | (0.01) | (0.05) | |
| L85R, K89N, A91T, F92P, K93V, | | | | | |
| R94L, I118V, T120S, I127T, T130A | | | | | |
| R29D, I30V, Y31L, Q33H, K36G, | 1422 | 1914 | 2024 | 179536 | 127 (0.28) |
| M38I, T41A, M43R, M47T, E81V, | | (0.04) | (0.01) | (0.04) | |
| L85R, K89N, A91T, F92P, K93V, | | | | | |
| R94L, I118V, T120S, I127T, | | | | | |
| T130A, H188D | | | | | |
| R29D, Y31L, Q33H, K36G, M38I, | 1424 | 2377 | 2177 | 438352 | 203 (0.45) |
| T41A, M43R, M47T, L70Q, E81V, | | (0.05) | (0.01) | (0.05) | |
| L85R, K89N, A91T, F92P, K93V, | | | | | |
| R94L, I118V, T120S, I127T, | | | | | |
| T130A, K169E | | | | | |
| R29D, Y31L, Q33H, K36G, M38I, | 1425 | 2106 | 2122 | 14201 | 226 (0.50) |
| T41A, M43R, M47T, L70Q, E81V, | | (0.05) | (0.01) | (0.05) | |
| K89N, A91T, F92P, K93V, R94L, | | | | | |
| I118V, T120S, I127T, T130A | | | | | |
| R29D, Y31L, Q33H, K36G, M38I, | 1426 | 1887 | 2201 | 110092 | 231 (0.51) |
| T41A, M43R, M47T, L85R, K89N, | | (0.04) | (0.01) | (0.04) | |
| A91T, F92P, K93V, R94L, I118V, | | | | | |
| T120S, I127T, T130A, K169E, | | | | | |
| H188D | | | | | |
| R29D, I30V, Y31L, Q33H, K36G, | 1422 | 2060 | 2385 | 94786 | 237 (0.53) |
| M38I, T41A, M43R, M47T, E81V, | | (0.05) | (0.01) | (0.05) | |
| L85R, K89N, A91T, F92P, K93V, | | | | | |
| R94L, I118V, T120S, I127T, | | | | | |
| T130A, H188D | | | | | |
| R29D, Y31L, Q33H, K36G, M38I, | 1427 | 2009 | 2623 | 110589 | 165 (0.37) |
| T41A, M43R, M47T, E81V, L85R, | | (0.04) | (0.01) | (0.04) | |
| K89N, A91T, F92P, K93V, R94L, | | | | | |
| F108L, I118V, T120S, T130A, | | | | | |
| K169E, H188D | | | | | |
| R29D, Y31L, Q33H, K36G, M38I, | 1312 | 1925 | 2979 | 379558 | 213 (0.47) |
| T41A, M43R, M47T, L70Q, E81V, | | (0.04) | (0.01) | (0.04) | |
| L85R, K89N, A91T, F92P, K93V, | | | | | |
| R94L, T130A, H188D | | | | | |
| R29D, Y31L, Q33H, K36G, M38I, | 1428 | 2245 | 2842 | 631549 | 118 (0.26) |
| T41A, M43R, M47T, L70Q, E81V, | | (0.05) | (0.01) | (0.05) | |
| L85R, K89N, A91T, F92P, K93V, | | | | | |
| R94L, I118V, T120S, T130A, | | | | | |
| N149D, K169E, H188D | | | | | |
| H18L, R29D, Y31L, Q33H, K36G, | 1429 | 2759 | 2247 | 760438 | 157 (0.35) |
| M38I, T41A, M43R, M47T, L70Q, | | (0.06) | (0.01) | (0.06) | |
| E81V, L85R, K89N, A91T, F92P, | | | | | |
| K93V, R94L, I118V, T120S, | | | | | |
| T130A, K169E, H188D | | | | | |
| R29D, Y31L, Q33H, K36G, M38I, | 1430 | 1585 | 2736 | 456003 | 278 (0.62) |
| T41A, M43R, M47T, E81V, L85R, | | (0.03) | (0.01) | (0.03) | |
| K89N, A91T, F92P, K93V, R94L, | | | | | |
| I118V, T120S, I127T, C128Y, | | | | | |
| T130A, H188D | | | | | |
| R29D, Y31L, Q33H, K36G, M38I, | 1316 | 2633 | 3379 | 133095 | 190 (0.42) |
| T41A, M43R, M47T, E81V, L85R, | | (0.06) | (0.01) | (0.06) | |
| K89N, A91T, F92P, K93V, R94F, | | | | | |
| T130A, K169E | | | | | |
| H18L, R29D, Y31L, Q33H, K36G, | 1431 | 1732 | 2082 | 117465 | 174 (0.39) |
| M38I, T41A, M43R, M47T, E81V, | | (0.04) | (0.01) | (0.04) | |
| L85R, K89N, A91T, F92P, K93V, | | | | | |
| R94L, E99D, T130A | | | | | |
| H18L, R29D, Y31L, Q33H, K36G, | 1432 | 2011 | 2502 | 711479 | 232 (0.51) |
| M38I, T41A, M43R, M47T, L70Q, | | (0.04) | (0.01) | (0.04) | |
| E81V, L85R, K89N, A91T, F92P, | | | | | |
| K93V, R94L, I118V, T120S, | | | | | |
| T130A, K169E | | | | | |
| R29D, Y31L, Q33H, K36G, M38I, | 1319 | 2026 | 2443 | 572017 | 202 (0.45) |
| T41A, M43R, M47T, L70Q, E81V, | | (0.04) | (0.01) | (0.04) | |
| L85R, K89N, A91T, F92P, K93I, | | | | | |
| R94L, L97R, T130A | | | | | |
| R29D, Y31L, Q33H, K36G, M38I, | 1320 | 1296 | 2119 | 777509 | 101 (0.22) |
| T41A, M43R, M47T, L70Q, E81V, | | (0.03) | (0.01) | (0.03) | |
| L85R, K89N, A91T, F92P, K93I, | | | | | |
| R94L, L97R, T130A, L148S | | | | | |
| H18L, R29D, Y31L, Q33H, K36G, | 1321 | 1188 | 2161 | 190176 | 97 (0.22) |
| M38I, T41A, M43R, M47T, E81V, | | (0.03) | (0.01) | (0.03) | |
| L85R, K89N, | | | | | |
| A91T, F92P, K93V, R94L, I118V, | | | | | |
| T120S, I127T, T130A, K169E | | | | | |
| R29D, Y31L, Q33H, K36G, M38I, | 1433 | 1203 | 1863 | 217243 | 288 (0.64) |
| T41A, M43R, M47T, I61N, E81V, | | (0.03) | (0.01) | (0.03) | |
| L85R, K89N, A91T, F92P, K93V, | | | | | |
| R94F, V104A, I118V, T120S, | | | | | |
| I126V, T130A | | | | | |
| R29D, Y31L, Q33H, K36G, M38I, | 1434 | 1289 | 2625 | 124188 | 111 (0.25) |
| T41A, M43R, M47T, E81V, L85R, | | (0.03) | (0.01) | (0.03) | |
| K89N, A91T, F92P, K93V, R94F, | | | | | |
| I118V, T120S, T130A | | | | | |
| R29D, Y31L, Q33H, K36G, M38I, | 1435 | 1228 | 1973 | 145285 | 114 (0.25) |
| T41A, M43R, M47T, T62S, E81V, | | (0.03) | (0.01) | (0.03) | |
| L85R, K89N, A91T, F92P, K93V, | | | | | |
| R94L, I118V, T120S, T130A, | | | | | |
| K169E, T175A | | | | | |
| H18L, R29D, Y31L, Q33H, K36G, | 1325 | 1244 | 2091 | 109646 | 114 (0.25) |
| M38I, T41A, M43R, M47T, E81V, | | (0.03) | (0.01) | (0.03) | |
| L85R, K89N, A91T, F92P, K93V, | | | | | |
| R94L, F116S, T130A, H188D | | | | | |
| H18L, R29D, Y31L, Q33H, K36G, | 1436 | 1221 | 2251 | 89654 | 143 (0.32) |
| M38I, T41A, M43R, M47T, E81V, | | (0.03) | (0.01) | (0.03) | |
| L85R, K89N, A91T, F92P, K93V, | | | | | |
| R94L, I118V, T120S, I127T, | | | | | |
| T130A, L142S, H188D | | | | | |
| C16S, H18L, R29D, Y31L, Q33H, | 1437 | 1212 | 1800 | 4497 | 247 (0.55) |
| K36G, M38I, T41A, M43R, M47T, | | (0.03) | (0.01) | (0.03) | |
| E81V, L85R, K89N, A91T, F92P, | | | | | |
| K93V, R94L, T110A, I118V, | | | | | |
| H188D | | | | | |
| R29D, Y31L, Q33H, K36G, M38I, | 1438 | 2620 | 2241 | 66183 | 125 (0.28) |
| T41A, M43R, M47T, A91G, I118V, | | (0.06) | (0.01) | (0.06) | |
| T120S, I127T, T130A, H188D | | | | | |
| R29D, Y31L, Q33H, K36G, M38I, | 1439 | 1907 | 1726 | 3508 | 224 (0.50) |
| T41A, M43R, M47T, L70Q, D76G, | | (0.04) | (0.01) | (0.04) | |
| A91G, S103L, I118V, T120S, | | | | | |
| I127T, T130A | | | | | |
| Y53C, L85R, K89N, A91T, F92P, | 1440 | 1396 | 1459 | 2552 | 75 (0.17) |
| K93V, R94L, I118V, T120S, I127T, | | (0.03) | (0.01) | (0.03) | |
| T130A, K169E | | | | | |
| T62S, E81V, L85R, K89N, A91T, | 1441 | 2947 | 2377 | 179622 | 107 (0.24) |
| F92P, K93V, R94L, I118V, T120S, | | (0.06) | (0.01) | (0.06) | |
| T130A, K169E | | | | | |
| L70Q, A91G, I118V, T120S, | 1386 | 12262 | 90597 | 4168 | 115 (0.25) |
| T130A, K169E | | (0.27) | (0.01) | (0.27) | |
| R29D, Y31L, Q33H, K36G, M38I, | 1332 | 1696 | 9960 | 4067 | 87 (0.19) |
| T41A, M43R, M47T, E81V, L85R, | | (0.04) | (0.01) | (0.04) | |
| K89N, A91T, F92P, K93V, R94L, | | | | | |
| S129L, H188D | | | | | |
| Y53C/L70Q/D90G/T130A/N149D/ | 1442 | 1990 | 3106 | 3289 | 122 (0.27) |
| N152T/H188D | | (0.04) | (0.01) | (0.04) | |
| WT CD80 | 2 | 45607 | 883950 | 93079 | 451 (1.00) |
| | | (1.00) | (1.00) | (1.00) | |
| H18L, R29D, Y31L, Q33H, K36G, | 1443 | 16232 | 140241 | 182403 | 108 (0.96) |
| M38I, T41A, M43R, M47T, E81V, | | (0.26) | (0.88) | (2.61) | |
| L85R, K89N, A91T, F92P, K93V, | | | | | |
| R94L, I118V, T120S, I127T, | | | | | |
| T130A, H188D | | | | | |
| K89E, T130A | 1465 | 46923 | 225651 | 196544 | 317 (2.81) |
| | | (0.75) | (1.41) | (2.82) | |
| K89E, K93E, T130A | 1335 | 39137 | 181037 | 206713 | 571 (5.06) |
| | | (0.63) | (1.13) | (2.96) | |
| K89E, T130A | 1465 | 61349 | 156244 | 126961 | 2539 (22.53) |
| | | (0.99) | (0.98) | (1.82) | |
| WT CD80 | 2 | 62220 | 160148 | 69786 | 113 (1.00) |
| | | (1.00) | (1.00) | (1.00) | |
| S21P, R29D, Y31L, Q33H, K36G, | 1336 | 4467 | 4434 | 146638 | 137 (0.65) |
| M38I, T41A, M43R, M47T, N48I, | | (0.20) | (0.02) | (5.87) | |
| V68A, E81V, L85R, K89N, A91T, | | | | | |
| F92P, K93V, R94L,P109H, I126L, | | | | | |
| K169I | | | | | |
| H18L, R29D, Y31L, Q33H, K36G, | 1337 | 4523 | 4565 | 4731 | 18 (0.09) |
| M38I, T41A, M43R, M47T, P74L, | | (0.21) | (0.02) | (0.19) | |
| Y80N, E81V, L85R, K89N, A91T, | | | | | |
| F92P, K93V, R94L, L97R | | | | | |
| R29D, Y31L, Q33H, K36G, M38I, | 1338 | 4675 | 4686 | 4098 | 54 (0.26) |
| T41A, M43R, M47T, E81V, L85R, | | (0.21) | (0.02) | (0.16) | |
| K89N, A91T, F92P, K93V, R94L, | | | | | |
| S21P, P74L, Y80N, D90N, T130A, | | | | | |
| N149S, E162G | | | | | |
| R29D, Y31L, Q33H, K36G, M38I, | 1339 | 4413 | 4618 | 221788 | 51 (0.24) |
| T41A, M43R, M47T, E81V, L85R, | | (0.20) | (0.02) | (8.88) | |
| K89N, A91T, F92P, K93V, R94L, | | | | | |
| H18L, V68M, T130A | | | | | |
| R29D, Y31L, Q33H, K36G, M38I, | 1340 | 4354 | 4413 | 201513 | 80 (0.38) |
| T41A, M43R, M47T, E81V, L85R, | | (0.20) | (0.02) | (8.06) | |
| K89N, A91T, F92P, K93V, R94L, | | | | | |
| V68M, T130A, N149S, R190S | | | | | |
| R29D, Y31L, Q33H, K36G, M38I, | 1341 | 4381 | 4491 | 5075 | 8 (0.04) |
| T41A, M43R, M47T, E81V, L85R, | | (0.20) | (0.02) | (0.20) | |
| K89N, A91T, F92P, K93V, R94L, | | | | | |
| H18L, P74L, Y80N, T130A, R190S | | | | | |
| C16G, V22A, R29D, Y31L, Q33H, | 1342 | 4459 | 4582 | 4383 | 45 (0.21) |
| K36G, M38I, T41A, M43R, M47T, | | (0.20) | (0.02) | (0.18) | |
| E81V, L85R, K89N, A91T, F92P, | | | | | |
| K93V, R94L, V68M, D76G, I118T, | | | | | |
| T130A, S140T, N149S, K169I, | | | | | |
| H178R, N192D | | | | | |
| R29D, Y31L, Q33H, K36G, M38I, | 1343 | 4371 | 4613 | 247135 | 61 (0.29) |
| T41A, M43R, M47T, E81V, L85R, | | (0.20) | (0.02) | (9.89) | |
| K89N, A91T, F92P, K93V, R94F, | | | | | |
| E117V, I118T, N149S, S168G, | | | | | |
| H188Q | | | | | |
| V22A, R29D, Y31L, Q33H, K36G, | 1344 | 4222 | 4381 | 235307 | 96 (0.46) |
| M38I, T41A, M43R, M47T, E81V, | | (0.19) | (0.022) | (9.42) | |
| L85R, K89N, A91T, F92P, K93V, | | | | | |
| R94L, V68M, T130A | | | | | |
| R29D, Y31L, Q33H, K36G, M38I, | 1345 | 4216 | 4305 | 149085 | 30 (0.14) |
| T41A, M43R, M47T, E81V, L85R, | | (0.19) | (0.022) | (5.97) | |
| K89N, A91T, F92P, K93V, R94F, | | | | | |
| N64S, I118T, T130A, N149S, K169I | | | | | |
| R29D, Y31L, Q33H, K36G, M38I, | 1346 | 4361 | 4459 | 95558 | 88 (0.42) |
| T41A, M43R, M47T, E81V, L85R, | | (0.20) | (0.022) | (3.82) | |
| K89N, A91T, F92P, K93V, R94L, | | | | | |
| V22A, V68M, D115G, I118T, | | | | | |
| T130A, G133D, N149S | | | | | |
| S129P | 1347 | 4458 | 5914 | 6669 | 0 (0.0) |
| | | (0.20) | (0.03) | (0.27) | |
| A91G, S129P | 1348 | 5144 | 26635 | 13569 | 0 (0.0) |
| | | (0.23) | (0.12) | (0.54) | |
| I69T, L70Q, A91G, T120S | 1349 | 74296 | 203363 | 6242 | 509 (2.42) |
| | | (3.37) | (0.89) | (0.25) | |
| S129P | 1347 | 5086 | 8957 | 6603 | 125 (0.60) |
| | | (0.23) | (0.04) | (0.26) | |
| Y31H, S129P | 1350 | 4553 | 4874 | 5788 | 76 (0.36) |
| | | (0.21) | (0.02) | (0.23) | |
| WT CD80 | 2 | 22053 | 227691 | 24989 | 211 (1.00) |
| | | (1.00) | (1.00) | (1.00) | |
| T28A, R29D, Y31L, Q33H, K36G, | 1351 | 2636 | 2169 | 99658 | 116 (0.62) |
| M38I, T41A, M43R, M47T, E81V, | | (0.17) | (0.03) | (9.45) | |
| L85R, K89N, A91T, F92P, K93V, | | | | | |
| R94L, V104L, T130A, N149S | | | | | |
| H18L, R29D, Y31L, Q33H, K36G, | 1352 | 2680 | 2165 | 107747 | 125 (0.68) |
| M38I, T41A, M43R, M47T, E81V, | | (0.17) | (0.03) | (10.22) | |
| L85R, K89N, A91T, F92P, K93V, | | | | | |
| R94L, L97R, N149S, H188Q | | | | | |
| H18L, R29D, Y31L, Q33H, K36G, | 1444 | 2482 | 2179 | 100116 | 96 (0.52) |
| M38I, T41A, M43R, M47T, E81V, | | (0.16) | (0.03) | (9.50) | |
| L85R, K89N, A91T, F92P, K93V, | | | | | |
| R94L, L97R, T130A, N149S | | | | | |
| R29D, Y31L, Q33H, K36G, M38I, | 1354 | 2455 | 2084 | 11855 | 132 (0.71) |
| T41A, M43R, M47T, E81V, L85R, | | (0.15) | (0.03) | (1.12) | |
| K89N, A91T, F92P, K93V, R94L, | | | | | |
| H18L, V68A, T130A, N149S, T154I | | | | | |
| R29D, Y31L, Q33H, K36G, M38I, | 1355 | 2804 | 2310 | 216297 | 191 (1.03) |
| T41A, M43R, M47T, E81V, L85R, | | (0.18) | (0.03) | (20.51) | |
| K89N, A91T, F92P, K93V, R94L, | | | | | |
| A12G, V68A, L97R, T130A, L183H | | | | | |
| R29D, Y31L, Q33H, K36G, M38I, | 1356 | 2460 | 2188 | 109263 | 150 (0.81) |
| T41A, M43R, M47T, E81V, L85R, | | (0.15) | (0.03) | (10.36) | |
| K89N, A91T, F92P, K93V, R94L, | | | | | |
| I118T, T130A, S140T, N149S, | | | | | |
| K169S | | | | | |
| R29D, Y31L, Q33H, K36G, M38I, | 1357 | 2569 | 2198 | 100074 | 130 (0.70) |
| T41A, M43R, M47T, E81V, L85R, | | (0.16) | (0.03) | (9.49) | |
| K89N, A91T, F92P, K93V, R94L, | | | | | |
| I118T, T130A, N149S, K169I, | | | | | |
| Q193L | | | | | |
| R29D, Y31L, Q33H, K36G, M38I, | 1358 | 2500 | 2188 | 147900 | 124 (0.67) |
| T41A, M43R, M47T, E81V, L85R, | | (0.16) | (0.03) | (14.03) | |
| K89N, A91T, F92P, K93V, R94L, | | | | | |
| V22A, I118T, T130A, N149S | | | | | |
| R29D, Y31L, Q33H, K36G, M38I, | 1359 | 2615 | 2210 | 118150 | 89 (0.48) |
| T41A, M43R, M47T, E81V, L85R, | | (0.16) | (0.03) | (11.21) | |
| K89N, A91T, F92P, K93V, R94L, | | | | | |
| I118T, T130A, N149S | | | | | |
| R29D, Y31L, Q33H, K36G, M38I, | 1360 | 2444 | 2246 | 115420 | 101 (0.55) |
| T41A, M43R, M47T, E81V, L85R, | | (0.15) | (0.03) | (10.95) | |
| K89N, A91T, F92P, K93V, R94L, | | | | | |
| I118T, T130A, N149S, K169I | | | | | |
| R29D, Y31L, Q33H, K36G, M38I, | 1361 | 2378 | 2123 | 112712 | 114 (0.61) |
| T41A, M43R, M47T, E81V, L85R, | | (0.15) | (0.03) | (10.69) | |
| K89N, A91T, F92P, K93V, R94F, | | | | | |
| T130A, N149S, K169I | | | | | |
| I118T, C128R | 1362 | 3093 | 3180 | 2620 | 122 (0.66) |
| | | (0.19) | (0.03) | (0.25) | |
| Q27R, R29C, M42T, S129P, E160G | 1363 | 2827 | 2623 | 2326 | 139 (0.75) |
| | | (0.18) | (0.03) | (0.22) | |
| S129P, T154A | 1364 | 3062 | 2622 | 2606 | 156 (0.84) |
| | | (0.19) | (0.03) | (0.25) | |
| WT CD80 | 2 | 15948 | 75099 | 10544 | 185 (1.00) |
| | | (1.00) | (1.00) | (1.00) | |

### Example 26

### Pharmacokinetic/Pharmacodynamic Modeling and Dose Selection of CD80 Variant Molecules in Humans

This Example describes preclinical pharmacokinetic (PK) and pharmacodynamic (PD) modeling and simulation of exemplary tested variant CD80 IgV-Fc (inert) (H18Y/A26E/E35D/M47L/V68M/A71G/D90G; SEQ ID NO: 491) to inform dose selection in humans. The PK/PD relationship was modeled based on data obtained from non-tumor bearing mice and a syngeneic mouse tumor model of huPD-L1-expressing MC38 tumors, and human PK was predicted from cynomolgus monkey.

### A. Modeling of PK

### 1. PK Model

A PK model was designed as a two compartment model with intercompartmental distribution (Q) between the first compartment (Central Compartment, V1) and the second compartment (Peripheral Compartment, V2). To capture intraperitoneal (IP) or subcutaneous (SC) injection routes, a third compartment (Depot Compartment) was included with a first-order rate constant (Ka) linking the third compartment uni-directionally to the first compartment. The model also included a constant F to account for loss in the amount of drug available to diffuse from the third compartment (Depot Compartment) to the first compartment (Central Compartment, V1) due to IP or SC injection. The total amount of available drug in the third compartment (Depot Compartment) is described as F×Dose. For intravenous injections (IV), the third compartment (Depot Compartment) was omitted.

Drug clearance (CL) from the first compartment (Central Compartment, V1) was modeled differently for each species. In the mouse model, a time-dependent increase in clearance due to the formation of anti-drug antibodies was accounted for as follows:

when t≤6 days, CL=CL₀;

when t≥6 days, CLₜ=CL₀×(e^{β×(t-6 days)});
where t is time in days, CL₀ is the clearance at day 0, CLₜ is the clearance at day t, and β is a constant. For monkeys, linear clearance from the first compartment (Central Compartment, V1) was assumed.

a. Mouse Model of PK

Non-tumor bearing female C57BU6NJ mice were administered single doses of 20 or 100 μg of the exemplary variant CD80 IgV-Fc (H18Y/A26E/E35D/M47L/V68M/A71G/D90G) through intraperitoneal (IP) or intravenous injection (IV). Serial serum samples were analyzed for variant CD80 IgV-Fc concentration and the data were used to develop the mouse PK model.

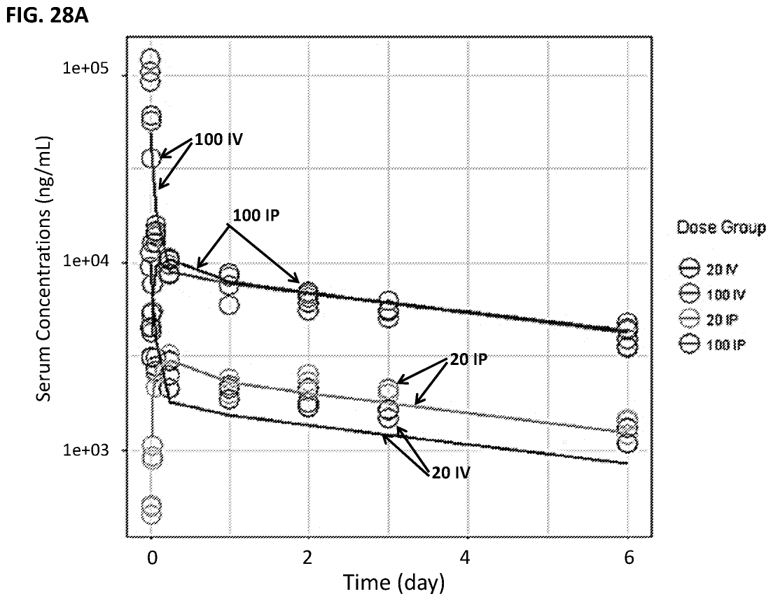

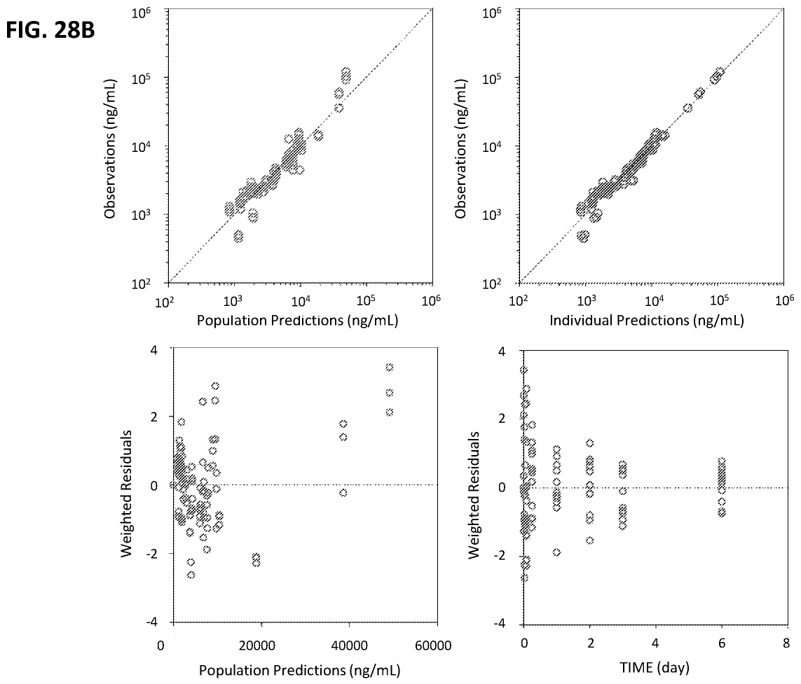

FIG. 28A shows observed (circles) and predicted (solid lines) serum concentration for dose groups over time. FIG. 28B shows the goodness of fit for the mouse PK model. The results indicate that the two-compartment model with a first-order absorption and time-dependent clearance describes well (predicts) the PK profiles in mice by either IV or IP injections.

PK was also compared in tumor-bearing versus non-tumor-bearing mice. Female C57BU6NJ mice were implanted with murine colon adenocarcinoma (MC38) cells expressing human PD-L1 and animals were randomly assigned into treatment groups in two dose ranging studies when individual animal tumor volume reached ˜50 mm³. In one study (Study #1), groups of individual mice were administered either a single dose of 100 μg of the exemplary variant CD80 IgV-Fc (H18Y/A26E/E35D/M47L/V68M/A71G/D90G) or a single dose of 33 μg of the exemplary variant CD80 IgV-Fc (H18Y/A26E/E35D/M47L/V68M/A71G/D90G) every 7 days for a total of 3 doses (Q7D×3 doses). In another study (Study #2), groups of individual mice were administered a single dose of 100, 500, or 1500 μg of the exemplary variant CD80 IgV-Fc (H18Y/A26E/E35D/M47L/V68M/A71G/D90G), or were administered 167 μg of the exemplary variant CD80 IgV-Fc (H18Y/A26E/E35D/M47L/V68M/A71G/D90G) every 3 days for a total of 3 doses (Q3D×3 doses). All treatments were administered IP. PK was determined in tumor-bearing mice in all groups and compared to the predicted PK in non-tumor bearing mice.

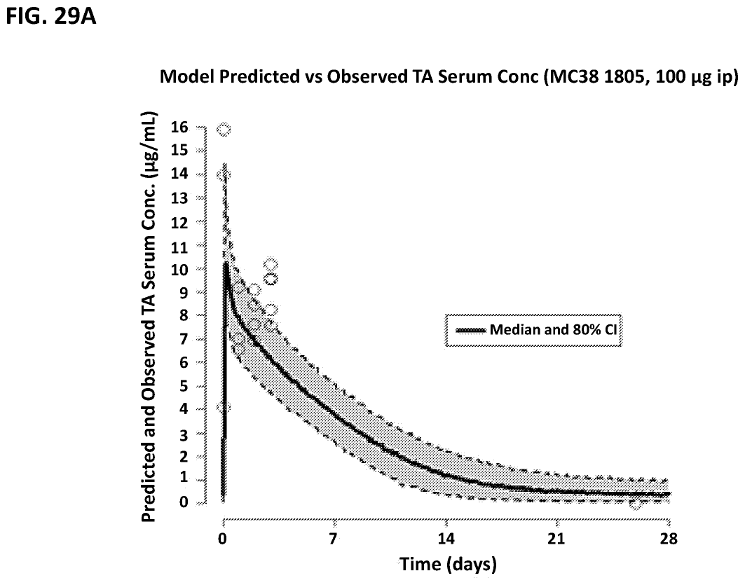

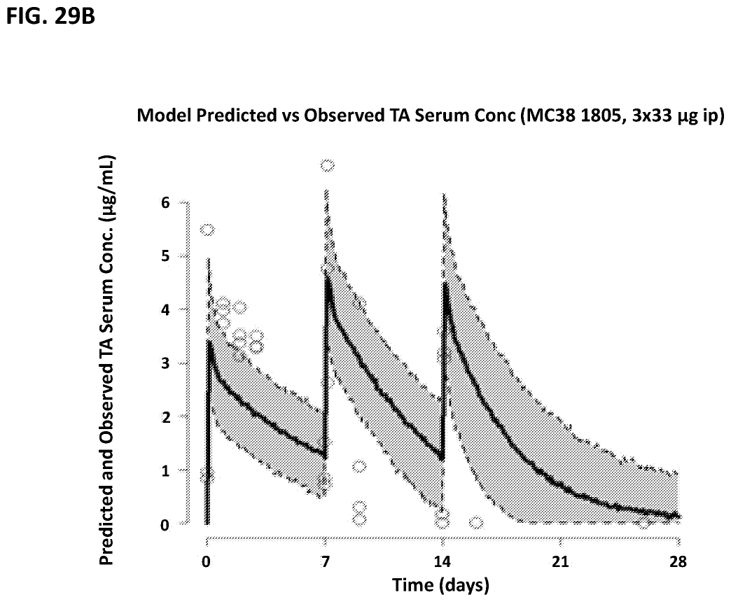

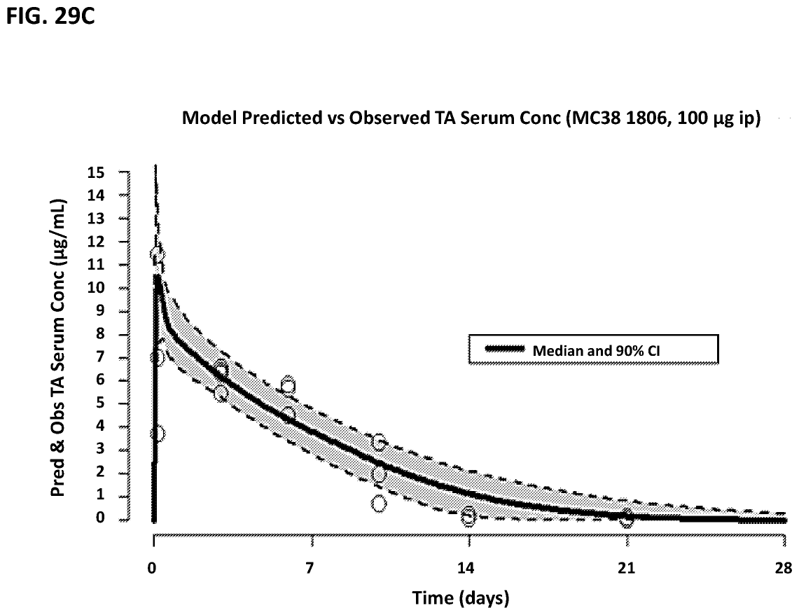

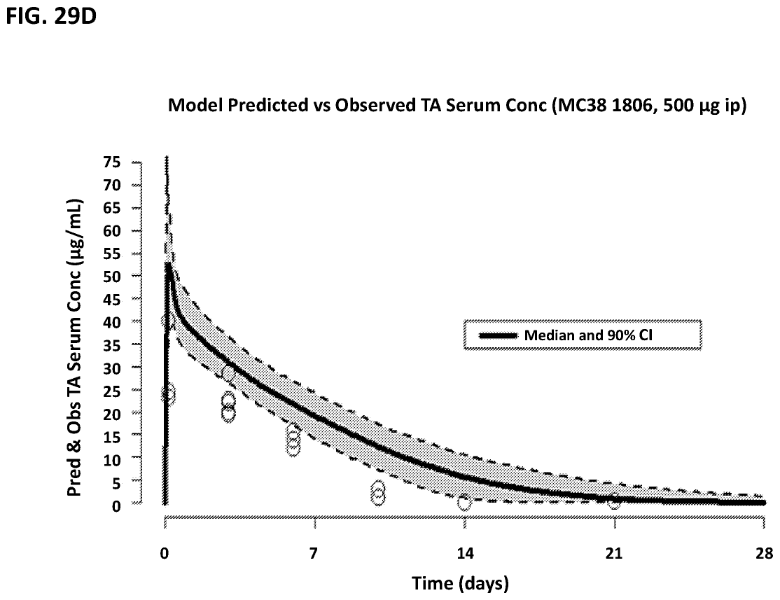

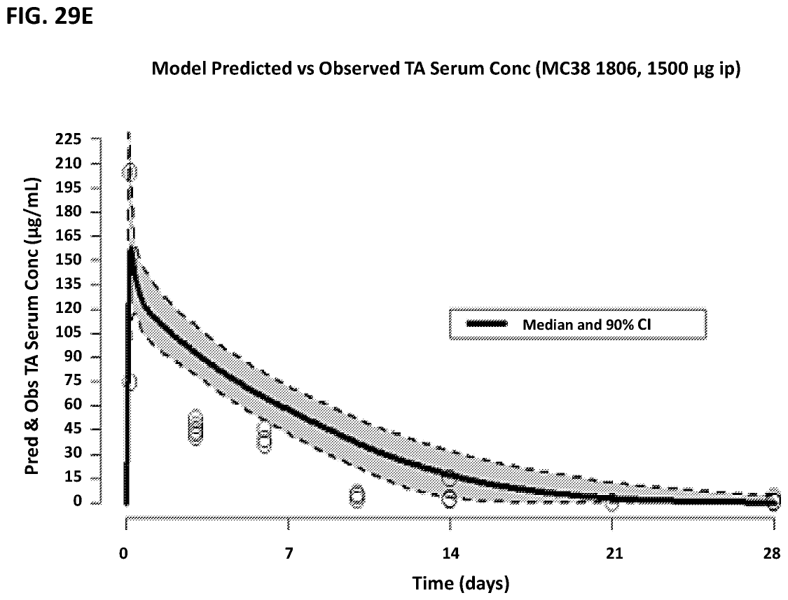

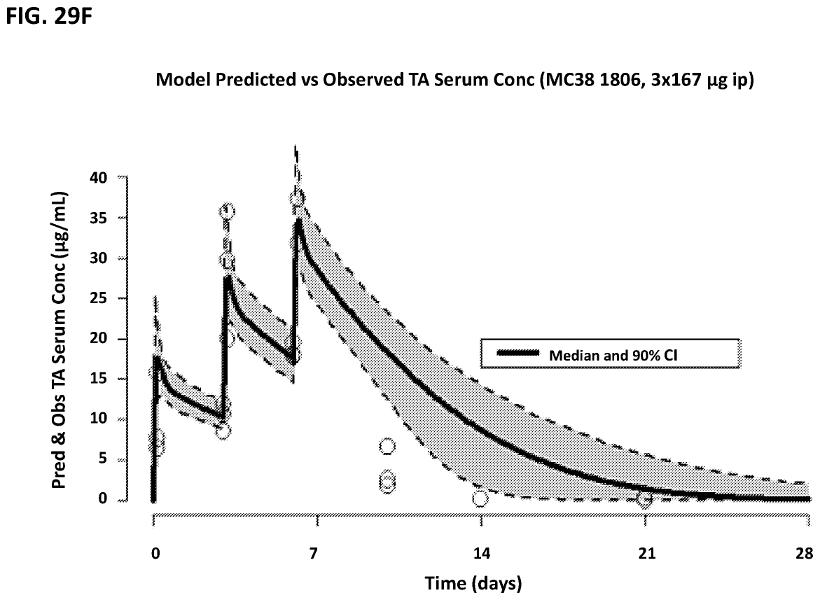

FIGS. 29A-29F show observed (circles) and predicted (solid lines) serum concentration, including confidence intervals (dashed lines), for treatment groups over time. These data show that PK of the exemplary variant CD80 IgV-Fc (H18Y/A26E/E35D/M47L/V68M/A71G/D90G) in hPD-L1-MC38 tumor-bearing mice overlaps with the PK in the non-tumor bearing PK model prediction. These results are consistent with a finding that PK in tumor-bearing and non-tumor bearing mice are similar, such that PK parameters obtained from non-tumor bearing mice can be used for tumor PK/PD model.

TABLE E35
provides estimated PK parameters for the
mouse PK model.
Estimated PK parameters for mouse PK model

| | Mouse | |
|---|---|---|
| Parameter | Estimates | Unit |
| Ka | 4.53 | Day⁻¹ |
| V1 | 1.94 | mL |
| V2 | 8.88 | mL |
| CL | 1.34 | mL/day |
| Q | 26.5 | mL/day |
| β | 0.0685 | Day⁻¹ |

| | |
|---|---|
| F | 99% |

b. Monkey Model of PK

Female cynomolgus monkeys were administered single doses of 0.1, 1, and 10 mg/kg of the exemplary variant CD80 IgV-Fc (H18Y/A26E/E35D/M47L/V68M/A71G/D90G) by IV infusion (30 min) or 10 mg/kg of the exemplary variant CD80 IgV-Fc (H18Y/A26E/E35D/M47L/V68M/A71G/D90G) by SC injection. Serial serum samples were analyzed for concentration of the variant CD80 IgV-Fc fusion protein and the data were used to develop the monkey PK model.

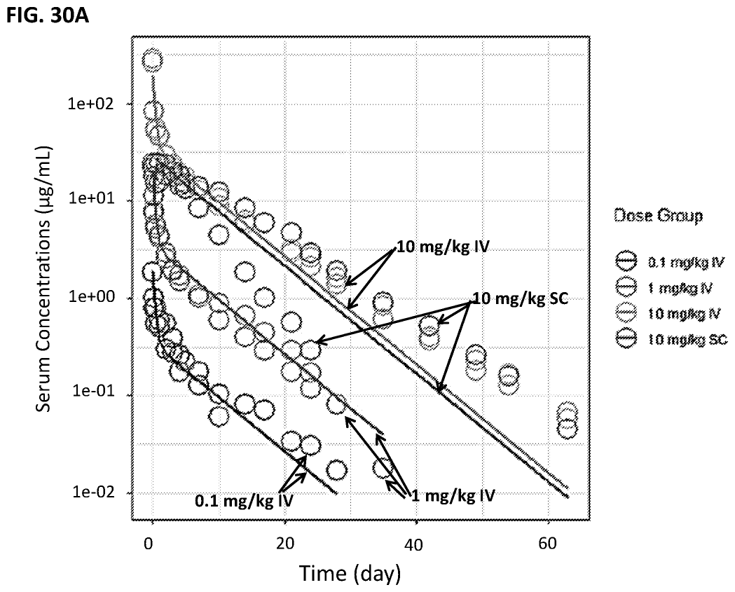

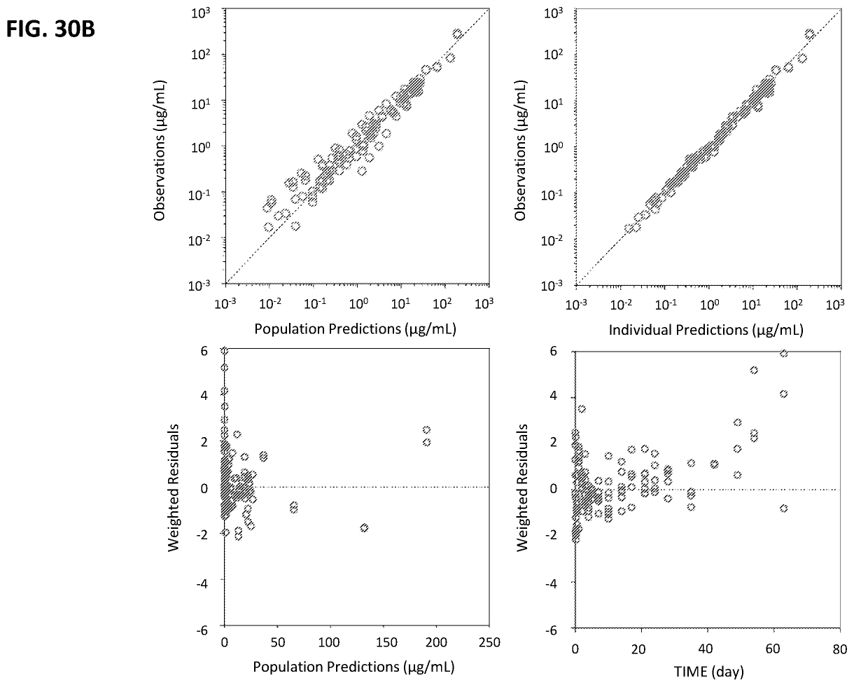

FIG. 30A shows observed (circles) and predicted (solid lines) serum concentration for each dose group over time. FIG. 30B shows the goodness of fit for the monkey PK model. These results indicate that the two-compartment model with linear clearance can characterize PK in monkey.

TABLE E36
provides estimated PK parameters for the monkey
PK model.
Estimated PK parameters for monkey PK model

| | Monkey | |
|---|---|---|
| Parameter | Estimates | Unit |
| Ka | 0.728 | Day⁻¹ |
| V1 | 50.9 | mL/kg |
| V2 | 158 | mL/kg |
| CL | 31.4 | mL/kg/day |
| Q | 105 | mL/kg/day |

| | |
|---|---|
| F | 66.4% |

### B. Modeling of PD

### 1. Mouse Model of PD

To model PD in a mouse tumor model, a sequential parameter estimation procedure, using NONMEM v7.4.2, was utilized, in which mouse PK parameters were fixed and PD parameters were estimated using mouse tumor data. A model of tumor growth was characterized by using individual animal tumor growth data from vehicle control treated groups.

Female CD57BU6NJ mice were implanted with murine colon adenocarcinoma MC38 cells expressing human PD-L1, assigned into treatment groups and dosed with exemplary variant CD80 IgV-Fc (H18Y/A26E/E35D/M47L/V68M/A71G/D90G) as described above in study #1 and study #2.

PD was modeled according to the schematic below, in which a signal distribution model (SDM) was employed to account for the tumor growth function and tumor growth inhibition function in the presence of the exemplary tested variant CD80 IgV-Fc (see, e.g., Lobo, E D et al., AAPS PharmSci. 2002; 4(4):E42).

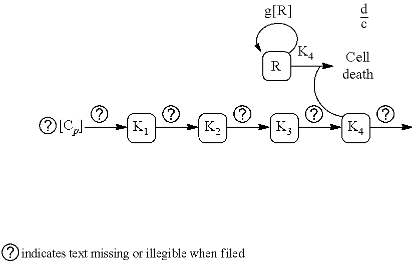

The SDM for PD included the following equations:

$$\frac{d R}{d T} = K g \times R - K 4 \times R \\ \frac{d K 1}{d t} = \frac{K - K 1}{τ} \\ \frac{d K 2}{d t} = \frac{K 1 - K 2}{τ} \\ \frac{d K 3}{d t} = \frac{K 2 - K 3}{τ} \\ \frac{d K 4}{d t} = \frac{K 3 - K 4}{τ} \\ K = \frac{K max \times {C}^{γ}}{{IC}_{50}^{γ} + {C}^{γ}}$$

where R represents tumor volume in mm³; K_{g} (day⁻¹) represents a first-order rate constant for net tumor growth; Kₘₐₓ (day⁻¹) represents the maximum kill rate constant; IC₅₀ (μg/mL) represents the drug concentration producing 50% of Kₘₐₓ; τ (day) is the mean transit time between compartments (e.g., K₁, K₂, K₃, and K₄, see schematic); γ is the Hill coefficient; f[Cp] (see schematic above) is the function of central compartment drug concentration change with time; a and g[R] (see schematic above) is the function of tumor growth change with time. In this model, the initial drug effect signal K is drug-concentration dependent. The initial effect signal is transduced through a cascade of transit compartments. At the end of transduction cascade, the initial drug effect results in the death of fraction of tumor cells.

A tumor static concentration (TSC), the minimum drug concentration where the tumor system is neither growing nor regressing, was calculated by the estimated PD parameters (Jumbe N L et al., J Pharmacokinet Pharmacodyn. 2010; 37:221), as follows:

$$TSC = {{IC}_{50} ( \frac{K g}{K max - K g} )}^{1 / γ}$$

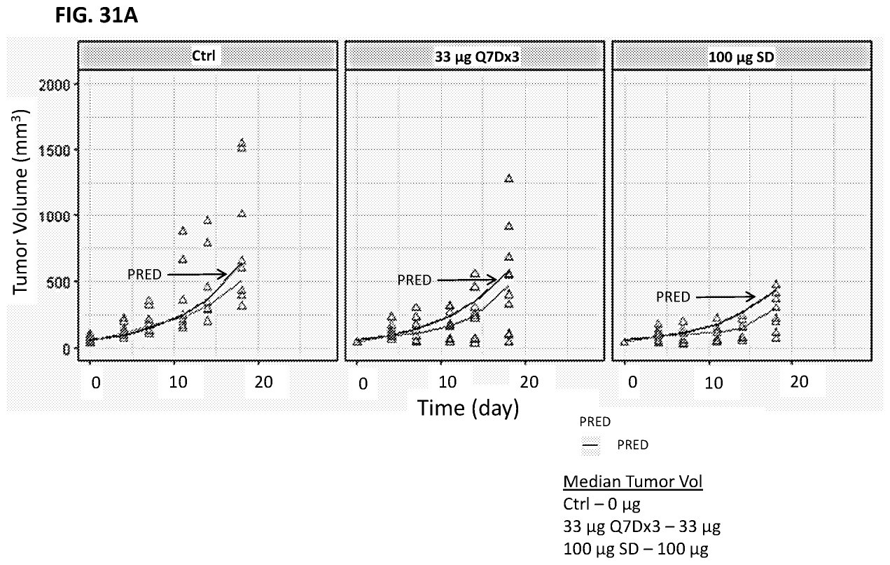

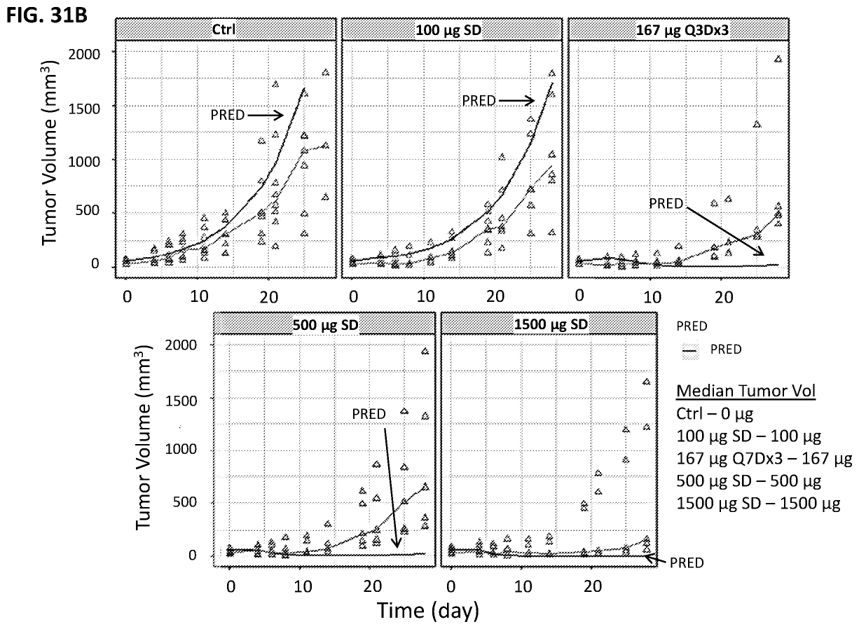

FIGS. 31A (study #1) and 31B (study #2) show observed (triangles with line fit) and predicted (solid lines) median tumor volume (mm³) for dose groups over days. Estimated PK parameters are set forth in Table E37. The results are supportive of the ability of a simple exponential tumor growth model to describe the hPD-L1-MC38 tumor growth data; the estimated tumor doubling time was approximately 5.3 days. The delayed tumor growth inhibition by the exemplary variant CD80 IgV-Fc was adequately described by the SDM. The TSC was calculated as 10.6 μg/mL. The results are consistent with an observation that inhibition of tumor growth by the exemplary variant CD80 IgV-Fc (H18Y/A26E/E35D/M47L/V68M/A71G/D90G) is both dose- and exposure-dependent.

TABLE E37
shows estimated PD parameters.
Estimated PD parameters for mouse PD model

| Parameter | Value | Unit |
|---|---|---|
| Wₒ | 56.6 | mm³ |
| K_{g} | 0.135 | Day⁻¹ |
| Kₘₐₓ | 0.689 | Day⁻¹ |
| IC₅₀ | 19.5 | μg/mL |
| γ | 2.32 | |
| τ | 0.65 | day |

### C. Predicted Human PK and Dose Selection in First-In-Human Study

Human PK parameters were calculated based on allometric scaling of the estimated monkey PK parameters based on body weight, with scaling factor 0.8 for CL and Q, and a factor of 1 for the first and second compartments (V1 and V2, see section A above). A starting dose was determined based on predicted maximum human serum concentration and target saturation. Predicted human PK profiles in combination with model estimated PD target concentration (TSC) was used to predict dose regimens in humans. Modeling was based on an IV route of administration.

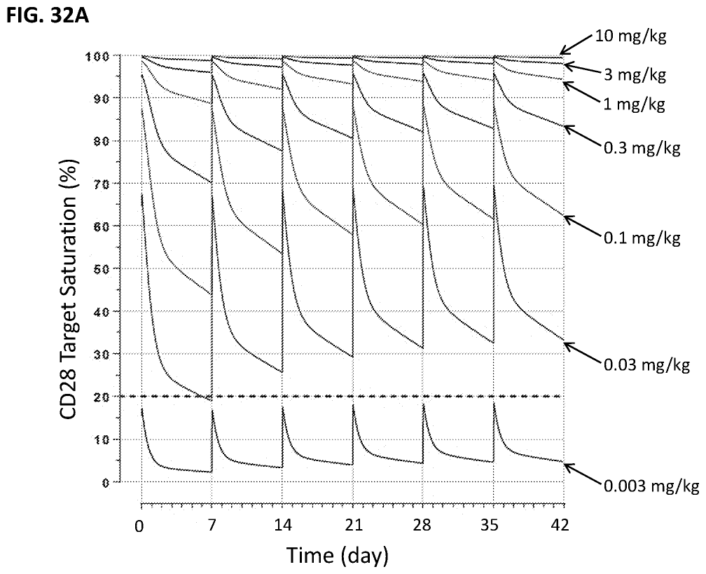

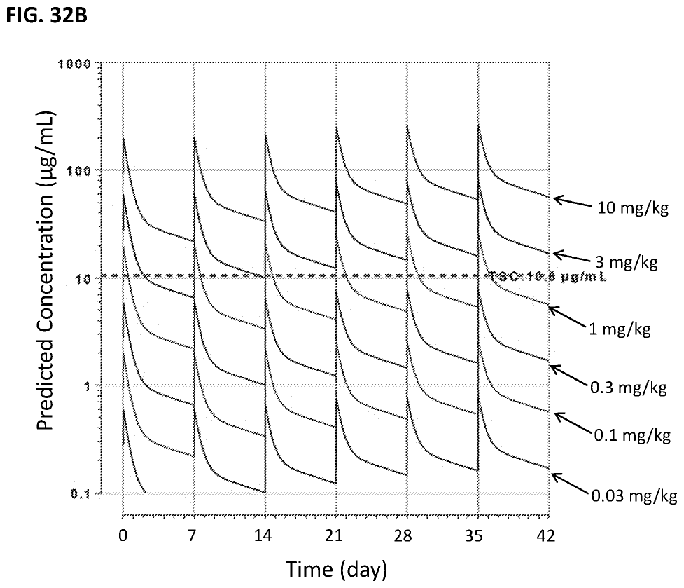

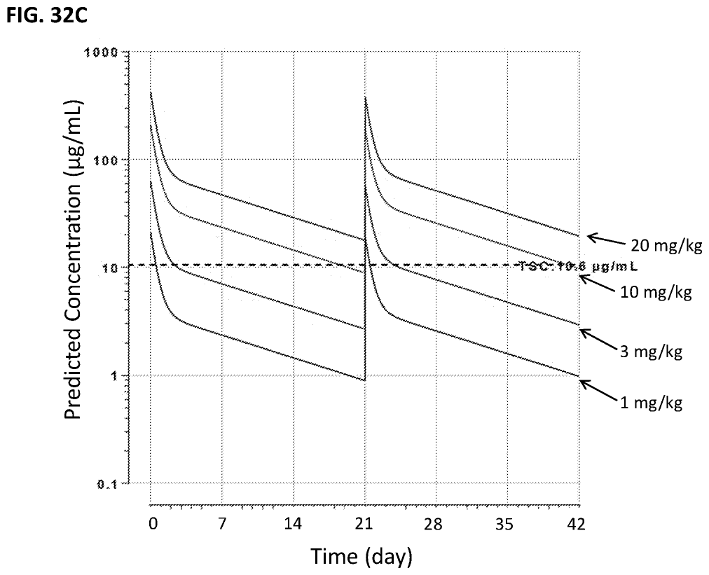

FIG. 32A shows the predicted target saturation with once weekly dosing (Q1W) of the exemplary tested variant CD80 IgV-Fc at different concentrations. Maximum target saturation was predicted to occur at the end of IV infusion. A starting dose of 0.003 mg/kg was determined to be the minimal anticipated biological effect level (MABEL) based on a predicted CD28 target saturation of 16% after the first IV dose of the variant CD80-IgV-Fc.

FIG. 32B shows the predicted human PK with once weekly dosing (Q1W) of the exemplary tested variant CD80 IgV-Fc at different concentrations, and FIG. 32C shows the predicted human PK with a dosing of the exemplary tested variant CD80 IgV-Fc every three weeks (Q3W) at different concentrations. Based on TSC, the projected clinical efficacious dose regimens were 1 to 3 mg/kg once a week (Q1W) and 3 to 10 mg/kg once every three weeks (Q3W).

### D. Conclusions

The translational strategy of combining non-clinical PK modeling/simulation and model-derived tumor static drug concentration (TSC) can be used to determine human dose selection. Transduction models, such as SDM described in this Example, may be used to inform immune-oncology biologic therapy.

### Example 27

### Assessment of Combination of CD80 Variant Molecule and Chemotherapeutic Agent in the MC38 Mouse Tumor Model

This Example describes the assessment of anti-tumor activity of exemplary tested variant CD80 IgV-Fc (inert) (variant CD80 IgV containing amino acid substitutions H18Y/A26E/E35D/M47L/V68M/A71G/D90G; SEQ ID NO: 491). To test the anti-tumor activity of the exemplary variant, it was evaluated alone or in combination with the platinum-based chemotherapeutic agent, oxaliplatin, in mice bearing human PD-L1-expressing MC38 tumors, which is a syngeneic mouse colon carcinoma solid tumor model.

The huPD-L1 MC38 cell line was generated by transducing parental MC38 cells with huPD-L1 using viral transduction. Eight-week old female C57/BL6NJ mice (The Jackson Laboratories, Sacramento, Calif.) were implanted subcutaneously with 0.5×10⁶ huPD-L1/MC38 cells. For injections, 0.1 mL of cells (0.5×10⁶ cells) were injected subcutaneously (SC) in the right mid-flank region.

On day 1, mice were randomized into four groups of 10 mice each, with all mice in each group having about equal mean tumor volumes (˜108 mm³). Tumors were measured with electronic calipers two-dimensionally twice weekly, beginning on day 7 post-tumor cell implant (referred to as “day 1”). Tumor volume was calculated as length×(width×2)×0.5, with the length being the longer of the two measurements. Beginning on day 1, the tested molecules were delivered through intraperitoneal (IP) injection, with a total of 3 doses of variant CD80 IgV-Fc delivered on days 1, 4, and 7 and oxaliplatin dosed on days 1, 8, and 15 as outlined in Table E38.

TABLE E38
Treatment Descriptions

| | | | | | Dose | Dose | |
|---|---|---|---|---|---|---|---|
| | | | | | Vol- | Schedule | Route |
| | | | | | ume | (D = | of |
| Group | # of | Test | Dose | | (mL/ | Study | Deliv- |
| # | Mice | Article(s) | Level | | kg) | Day) | ery |
| 1 | 10 | Fc control | 75 | μg | 5 | D1, D4, | IP |
| | | | | | | D7 | |
| 2 | 10 | Variant | 100 | μg | 5 | D1, D4, | IP |
| | | CD80 | | | | D7 | |
| | | IgV-Fc | | | | | |
| | | (H18Y/ | | | | | |
| | | A26E/ | | | | | |
| | | E35D/ | | | | | |
| | | M47L/ | | | | | |
| | | V68M/ | | | | | |
| | | A71G/D90; | | | | | |
| | | SEQ | | | | | |
| | | ID NO: | | | | | |
| | | 491) | | | | | |
| 3 | 10 | Fc control | 75 | μg | 5 | D1, D4, | IP |
| | | | | | | D7 | |
| | | Oxaliplatin | 5 | mg/kg | 10 | D1, D8, | IP |
| | | | | | | D15 | |
| 4 | 10 | Variant | 100 | μg | 5 | D1, D4, | IP |
| | | CD80 | | | | D7 | |
| | | IgV-Fc | | | | | |
| | | (H18Y/ | | | | | |
| | | A26E/ | | | | | |
| | | E35D/ | | | | | |
| | | M47L/ | | | | | |
| | | V68M/ | | | | | |
| | | A71G/D90; | | | | | |
| | | SEQ | | | | | |
| | | ID NO: | | | | | |
| | | 491) | | | | | |
| | | Oxaliplatin | 5 | mg/kg | 10 | D1, D8, | IP |
| | | | | | | D15 | |

Tumor growth inhibition (TGI) values for individual mice within each treatment group were calculated using tumor volumes from the last day in which all mice from each group were alive on study (day 25) using the following formula: [(mean Fc control tumor size −tumor size for individual mouse) divided by mean Fc control tumor size]×100.

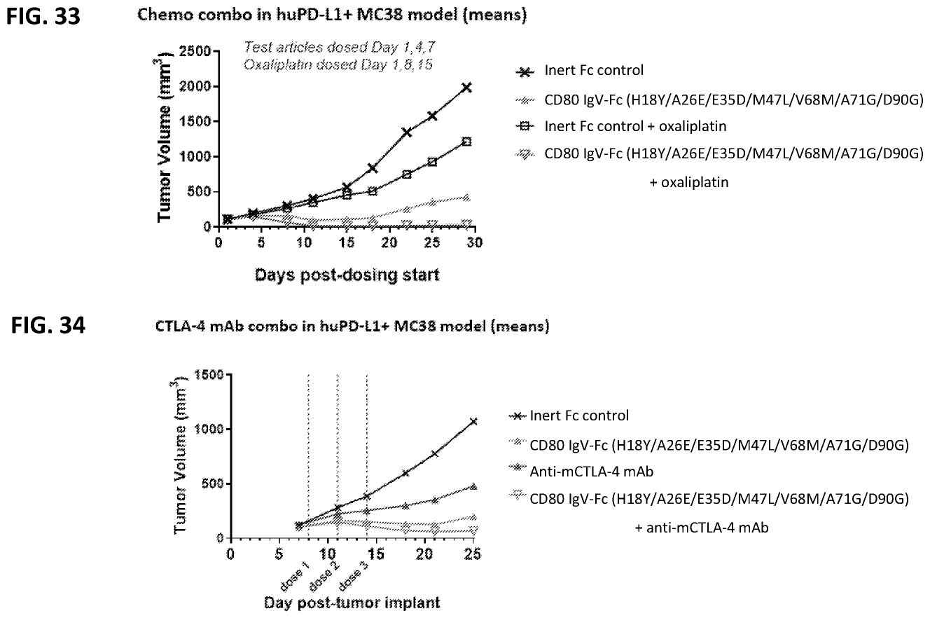

As shown in FIG. 33, oxaliplatin alone was only modestly effective as monotherapy in huPD-L1+MC38 tumor-bearing mice, while variant CD80 IgV-Fc (inert) (H18Y/A26E/E35D/M47L/V68M/A71G/D90G) had potent antitumor activity in this model. When the chemotherapy was administered in combination with CD80 IgV-Fc (inert) (H18Y/A26E/E35D/M47L/V68M/A71G/D90G), significantly greater reductions in tumor growth over time were observed than with either of the agents alone. Although all groups had equal-sized tumors at the start of treatment (108 mm³), the median tumor volume for the group tested with the combination of CD80 IgV-Fc (inert) (H18Y/A26E/E35D/M47L/V68M/A71G/D90G) and oxaliplatin dropped to zero by eleven days after start of dosing

A percent mean tumor growth inhibition (TGI) among individual mice treated was also determined based on tumor volume from the last day in which all groups had at least 70% of mice still alive on study (day 25), using the formula [(mean Fc control tumor size −mean treated tumor size) divided by mean Fc control tumor size]×100). The anti-tumor activity of the combination as measured by TGI is shown in Table E39. As shown, 30% of the mice in the monotherapy group were tumor free by the end of the study. Three weekly doses of oxaliplatin given in combination with Fc control resulted in less potent anti-tumor activity than that observed for the monotherapy. There were no tumor-free mice in the Fc control+oxaliplatin group, but the differences of tumor growth and mean TGI values in that group versus Fc control alone were statistically significant. The combination of weekly oxaliplatin treatments with variant CD80 IgV-Fc (inert) (H18Y/A26E/E35D/M47L/V68M/A71G/D90G) dosing resulted in significantly superior anti-tumor activity compared to all other groups, with 90% of mice becoming tumor free and a mean TGI value of nearly 100%.

TABLE E39
Summary of Anti-Tumor Growth Activity

| | | | | | Mean | # (%) TF |
|---|---|---|---|---|---|---|
| Group | Test | Dose | | Dose | % TGI | Mice |
| # | Article | Level | | Schedule | (D25) | (D32) |
| 1 | Fc control | 75 | μg | D1, D4, D7 | 0 | 0/10 (0) |
| 2 | Variant | 100 | μg | D1, D4, D7 | 77.5 | 3/10 |
| | CD80 | | | | | (33%) |
| | IgV-Fc | | | | | |
| | (H18Y/ | | | | | |
| | A26E/ | | | | | |
| | E35D/ | | | | | |
| | M47L/ | | | | | |
| | V68M/ | | | | | |
| | A71G/ | | | | | |
| | D90G; | | | | | |
| | SEQ | | | | | |
| | ID NO: | | | | | |
| | 491) | | | | | |
| 3 | Fc control | 75 | μg | D1, D4, D7 | 41.2 | 0/10 (0) |
| | Oxaliplatin | 5 | mg/kg | D1, D8, D15 | | |
| 4 | Variant | 100 | μg | D1, D4, D7 | 98.6 | 9/10 |
| | CD80 | | | | | (90%) |
| | IgV-Fc | | | | | |
| | (H18Y/ | | | | | |
| | A26E/ | | | | | |
| | E35D/ | | | | | |
| | M47L/ | | | | | |
| | V68M/ | | | | | |
| | A71G/ | | | | | |
| | D90G; | | | | | |
| | SEQ | | | | | |
| | ID NO: | | | | | |
| | 491) | | | | | |
| | Oxaliplatin | 5 | mg/kg | D1, D8, D15 | | |

TGI = tumor growth inhibition;
TF = tumor free;
D = day;
IP = intraperitoneal.

These results indicate that variant CD80 IgV-Fc (inert) molecules with increased binding affinity for PD-L1, such as the exemplary variant containing amino acid substitutions H18Y/A26E/E35D/M47L/V68M/A71G/D90G, can be administered in the presence of a chemotherapeutic agent such as the exemplary platinum-based chemotherapeutic agents oxaliplatin, and that efficacy may be enhanced with the combination treatment.

### Example 28

### Assessment of Combination of CD80 Variant Molecule and Anti-CTLA-4 Antibody in the MC38 Mouse Tumor Model

This Example describes the assessment of anti-tumor activity of exemplary tested variant CD80 IgV-Fc (inert) (variant CD80 IgV containing amino acid substitutions H18Y/A26E/E35D/M47L/V68M/A71G/D90G; SEQ ID NO: 491). To test the anti-tumor activity of the exemplary variant, the variant CD80 IgV-Fc was evaluated alone or in combination an exemplary anti-mouse checkpoint antibody against CTLA-4 (anti-CTLA-4; clone 9D9) in mice bearing human PD-L1-expressing MC38 tumors, which is a syngeneic mouse colon carcinoma solid tumor model.

Eight-week old female C57/BL6NJ mice (The Jackson Laboratories, Sacramento, Calif.) were implanted subcutaneously with 1.5×10⁶ huPD-L1/MC38 cells (described in Example 27. On days 8, 11 or 14 post-tumor cell implant mice, the tested molecules were delivered through intraperitoneal (IP) injection as set forth in Table E40. Tumor volume was determined substantially as described in Example 27.

TABLE E40
Treatment Description

| Group | # of | | Dose |
|---|---|---|---|
| # | Mice | Test Article(s) | Level |
| 1 | 9 | Fc control | 75 μg |
| 2 | 9 | Variant CD80 | 150 μg |
| | | IgV-Fc | |
| | | (H18Y/A26E/E3 | |
| | | 5D/M47L/V68M/ | |
| | | A71G/D90; SEQ | |
| | | ID NO: 491) | |
| 3 | 9 | Anti-CTLA-4 | 100 μg |
| 4 | 9 | Variant CD80 | 150 μg |
| | | IgV-Fc | |
| | | (H18Y/A26E/E3 | |
| | | 5D/M47L/V68M/ | |
| | | A71G/D90; SEQ | |
| | | ID NO: 491) | |
| | | Anti-CTLA-4 | 100 μg |

As shown in FIG. 34, anti-CTLA-4 alone was only modestly effective as monotherapy in huPD-L1+MC38 tumor-bearing mice, while variant CD80 IgV-Fc (inert) (CD80 IgV H18Y/A26E/E35D/M47L/V68M/A71G/D90G, SEQ ID NO:491) had potent antitumor activity in this model. When the anti-CTLA-4 checkpoint inhibitor was administered in combination with CD80 IgV-Fc (inert) (H18Y/A26E/E35D/M47L/V68M/A71G/D90G), a significantly greater reduction in tumor growth over time was observed than with either agent alone. These results are consistent with a finding that variant CD80 IgV-Fc (inert) molecules with increased binding affinity for PD-L1, such as the exemplary variant containing amino acid substitutions H18Y/A26E/E35D/M47L/V68M/A71G/D90G, is compatible with as a combination therapy with checkpoint inhibitors and can improve the antitumor activity of other only modestly effective treatment modalities, such as checkpoint-only blockades.

### Example 29

### Assessment of the Mechanism of Action of Variant CD80 IgV-Fc

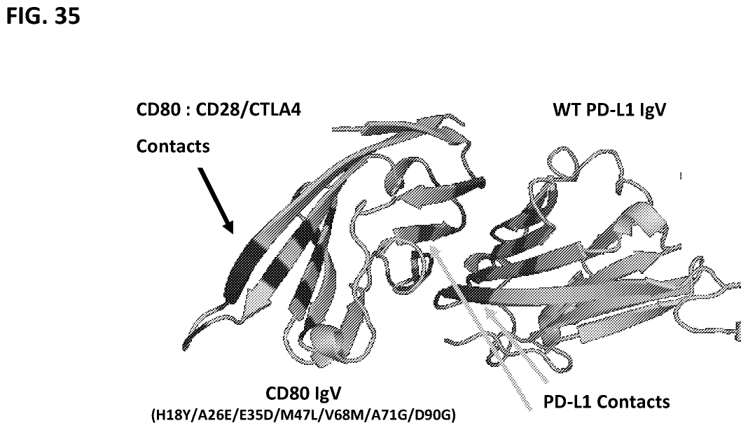

X-ray crystallography was used to elucidate the crystal structure of CD80 IgV domain of CD80 IgV-Fc (inert) (CD80 IgV H18Y/A26E/E35D/M47L/V68M/A71G/D90G; SEQ ID NO:491) to wild-type PD-L1. As shown in FIG. 35, the CD80 IgV:PD-L1 crystal structure (at a resolution of 3.15 Å) demonstrated a non-overlapping binding interface in which PD-L1 contact resides were distinct from the CD28:CD80 contacts. Residues involved in the interaction between CD80 and PD-L1 within 4.0 Å of each other were defined as interaction residues (M. D. Winn et al. Acta. Cryst. D 67, 235-242 (2011)) and included: CD80 residues Lys43, Glu44, Val45, Lys88, Asn89, Arg90, Thr91, Ile92, Asp94, Met102, Leu104, Gly105, Arg 107 and PD-L1 residues Ile54, Tyr56, Gln66, Val68, His69, Glu71, Arg113, Met115, Ile116, Ser 117, Gly120, Ala121, Asp122, Tyr123.

To further assess anti-tumor activity mechanistically, anti-tumor activity was evaluated in vivo using a human PD-L1-transduced MC38 tumor model substantially as described in Example 28, by combining CD80 IgV-Fc (inert) (H18Y/A26E/E35D/M47L/V68M/A71G/D90G; SEQ ID NO:491) treatment with anti-PD-L1 or anti-CD28 blocking antibodies. Anti-tumor activity was evaluated by serial tumor measurements. On days 8, 11 or 14 post-tumor cell implant mice, the tested molecules were delivered through intraperitoneal (IP) injection each alone or in combination at a concentration of 100 μg antibody or CD80 IgV-Fc.

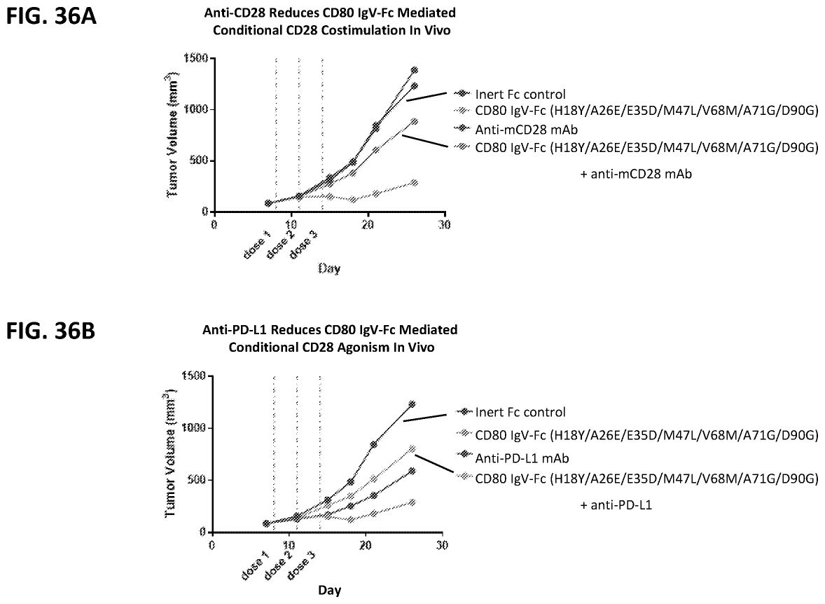

As shown in FIG. 36A (anti-CD28) or FIG. 36B (anti-PD-L1), the variant CD80 IgV-Fc (inert) (H18Y/A26E/E35D/M47L/V68M/A71G/D90G) demonstrated activity superior to PD-L1 blockade alone, and coadministration with either anti-CD28 or anti-PD-L1 reduced the antitumor activity of the variant CD80 IgV-Fc (inert) monotherapy.

Together, these results are consistent with an observation that the CD80 IgV domain utilizes separate, non-competing epitopes to bind CD28 and PD-L1. The exemplary variant CD80 IgV-Fc (inert) (H18Y/A26E/E35D/M47L/V68M/A71G/D90G) is able to simultaneously engage PD-L1 on tumor cells and CD28 or CTLA-4 on T cells, conferring the unique abilities to block both the PD-L1 and CTLA-4 checkpoints as well as eliciting CD28 costimulation in the presence of PD-L1. Further, the superior activity of the exemplary variant CD80 IgV-Fc (inert) (H18Y/A26E/E35D/M47L/V68M/A71G/D90G) over checkpoint inhibitor-only therapies, such as anti-PD-L1, indicates that the ability of the variant CD80 IgV-Fc (inert) to both mediate checkpoint inhibition and CD28 costimulation may result in improved anti-tumor responses.

### Example 30

### Generation of CD80 IgV-Fc Secreted Immunomodulatory Protein (SIP) and Assessment of SIP Secretion, Bioactivity, and Binding

To generate a CD80 IgV-Fc secreted immunomodulatory protein (SIP), DNA encoding exemplary SIPs was obtained as gene blocks from Integrated DNA Technologies (Coralville, USA) and then cloned by Gibson assembly (New England Biolabs Gibson assembly kit) into a modified version of pRRL vector between restriction sites downstream of MND promoter. Exemplary SIP constructs were generated to encode a protein, including the signal peptide, and additionally a tag moiety. Specifically, the vector also encoded GFP, and a furin-linker-P2a sequence was placed between the DNA encoding the SIP and the DNA encoding the GFP allowing contiguous expression of the SIP and GFP proteins. The gene blocks had the following structure in order: 53 base pair overlap with signal peptide sequence upstream of Afe1 restriction site (ATGGGGTCAACCGCCATCCTCGCCCTCCTCCTGGCTGTTCTCCAAGGAGTCAGCGCT (SEQ ID NO: 1545)), encoding the signal peptide set forth as MGSTAILALLLAVLQGVSA (SEQ ID NO: 1546)); DNA sequence encoding SIP amino acid sequence set forth in Table E41 below, also including in all cases one or more linkers set forth in SEQ ID NO:1504 (3×GGGGS) or SEQ ID NO: 1522 (GSGGGGS): DNA encoding a human IgG1 Fc region modified to eliminate Fc effector function (SEQ ID NO:1518); 38 base pair overlap with coding sequence downstream of a unique Blp1 restriction site within DNA encoding a Furin cleavage site (RAKR); additional ORF sequences cloned in downstream of the Furin cleavage site that included in order: DNA encoding linker sequence (SSGSGGSG, SEQ ID NO: 1593); DNA encoding ribosomal skipping sequence P2a (ATNFSLLKQAGDVEENPGP, SEQ ID NO: 1594); DNA encoding GFP (MVSKGEELFTGVVPILVELDGDVNGHKFSVSGEGEGDATYGKLTLKFICTTGKLPVPWPTLVTT LTYGVQCFSRYPDHMKQHDFFKSAMPEGYVQERTIFFKDDGNYKTRAEVKFEGDTLVNRIELKG IDFKEDGNILGHKLEYNYNSHNVYIMADKQKNGIKVNFKIRHNIEDGSVQLADHYQQNTPIGDGP VLLPDNHYLSTQSALSKDPNEKRDHMVLLEFVTAAGITLGMDELYK, SEQ ID NO: 1582); TAA stop codon.

Exemplary generated SIP constructs are set forth in Table E41. As shown, bivalent and other multivalent, e.g. tetravalent formats were generated, including SIP version of exemplary multivalent formats described in Example 24.

TABLE E41
CD80 IgV-Fc Secreted Immunomodulatory Protein (SIP) Variants

| | CD80 | | Inert | |
|---|---|---|---|---|
| | IgV | | Fc | |
| | SEQ | CD80 IgV | SEQ | Inert Fc |
| Description | ID NO | Mutations | ID NO | Mutations |
| Control SIP: | 150 | — | 1518 | C220S, |
| Wildtype | | | | L234A, |
| CD80 IgV- | | | | L235E, and |
| GSG4S-Inert | | | | G237A |
| Fc | | | | |
| SIP 1: | 491 | H18Y, A26E, E35D, | 1518 | C220S, |
| Variant CD80 | | M47L, V68M, A71G, | | L234A, |
| IgV-GSG4S- | | and D90G | | L235E, and |
| Inert Fc | | | | G237A |
| SIP 2: | 765 or | E35D, D46V, M47L, | 1518 | C220S, |
| Variant CD80 | 976 | and V68M | | L234A, |
| IgV-GSG4S- | | | | L235E, and |
| Inert Fc | | | | G237A |
| SIP 3: | 1176 | H18Y, E35D, M47V, | 1518 | C220S, |
| Variant CD80 | or | V68M, and A71G | | L234A, |
| IgV-GSG4S- | 1207 | | | L235E, |
| Inert Fc | | | | and G237A |
| SIP 4: | 1187 | H18Y, A26P, E35D, | 1518 | C220S, |
| Variant CD80 | or | M47I, V68M, | | L234A, |
| IgV-GSG4S- | 1218 | and A71G | | L235E, |
| Inert Fc | | | | and G237A |
| SIP 5: | 491 | H18Y, A26E, E35D, | 1518 | C220S, |
| Variant CD80 | | M47L, V68M, | | L234A, |
| IgV-GSG4S | | A71G, and D90G | | L235E, |
| Inert Fc- | | | | and G237A |
| 3xG4S- | | | | |
| Variant CD80 | | | | |
| IgV | | | | |
| SIP 6: | 491 | H18Y, A26E, E35D, | 1518 | C220S, |
| Variant CD80 | | M47L, | | L234A, |
| IgV-3xG4S- | | V68M, A71G, | | L235E, |
| Variant | | and D90G | | and G237A |
| CD80 IgV- | | | | |
| GSG4S-Inert | | | | |
| Fc | | | | |

To prepare lentiviral vectors, 4×10⁶ HEK293 cells were plated per 100 mm dish. On the next day, 4.5 μg of P-Mix (3 μg of PAX2 and 1.5 μg of pMD2G) was added to 6 μg of DNA encoding the SIPs constructs in a 5 mL polypropylene tube. Diluent buffer (10 mM HEPES/150 mM NaCl pH7.05/1L TC grade H20) was added to the tube to bring up the total volume of 500 μL. To the diluent DNA (PEI:total DNA 4:1), 42 μL of PEI (1 μg/μL) was added and mixed by vortexing. The mixture was incubated at room temperature for 10 minutes and cells were prepared by aspirating medium from the dish gently without disturbing the adherent cells, then replaced with 9 mL of Opti-MEM (1×). DNA/PEI mixture was then added to the dish and incubated at 37° C. for 24 hours. After 24 hours, media was aspirated from the dishes and replaced with 10 mL of fresh DMEM media and then incubated at 37° C. Viral supernatant was collected after 48 hours using a syringe attached to a 0.45 μm filter PES to remove cells and debris from the culture (Thermo Scientific Nalgene Syringe Filter).

Jurkat cells and donor Pan T-cells were transduced with the viral vectors encoding the CD80 IgV-Fc SIPs. Pan T-cells were thawed and activated with anti-CD3/anti-CD28 beads (Dynal) at a 1:1 ratio. Cells (1×10⁶ cells) were mixed with supernatant containing lentiviral particles encoding the indicated CD80 IgV-Fc SIPs with concentrations adjusted for a targeted MOI of 0.7. As a control, cells were transduced with a mock vector control. Transduction was performed in the presence of 8 μg/mL polybrene and 100 IU/mL IL-2. Cells were spun down at 1000 g for 30 min at 30° C. After 24 hours, 0.8 mL of media was removed from each well and replaced with fresh Xvivo15 plus media and IL2. The cells were fed every two days with fresh media and cytokines.

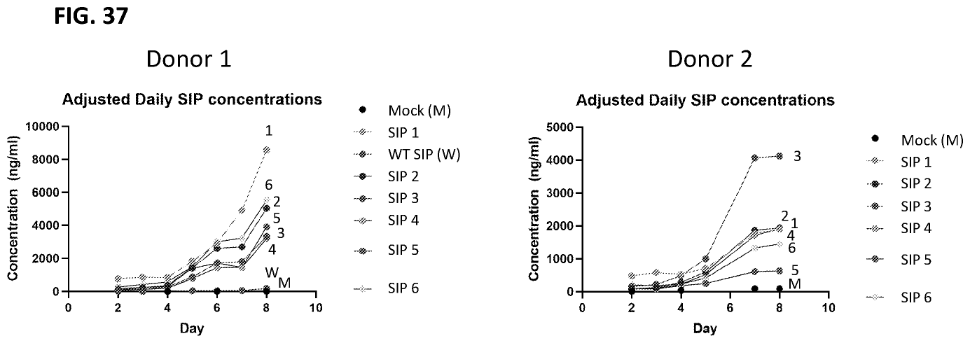

Starting on Day 2 after transduction, the culture supernatant from transduced cells above was collected daily for ELISA assays. Samples for ELISA assays included undiluted supernatant from transduced cells and 1:3 serial dilutions thereof, and a standard curve for determining supernatant SIP concentration was generated using 50 ng/mL of purified recombinant SIP protein and 1:3 serial dilutions thereof. As shown in FIG. 37, SIP proteins were detected in supernatant samples from transduced Pan T-cells from two different donors, but were not detected in supernatant samples from mock transduced cells.

To assess the capacity of CD80 IgV-Fc SIPs to modulate CD28 costimulation, a Jurkat/IL-2 reporter assay was performed substantially as described in Example 9. Jurkat effector cells expressing an IL-2-luciferase reporter were co-cultured with K562-derived artificial antigen presenting cells (aAPC) displaying transduced cell surface anti-CD3 single chain Fv (OKT3) and PD-L1. CD28 costimulation was assessed upon addition of a purified CD80 IgV-Fc protein control (SIP 1 from Table E41) starting at 25 nM and 1:3 serial dilutions thereof as well as undiluted supernatants and 1:3 serial dilutions thereof from SIP-transduced cells seven days after transduction.

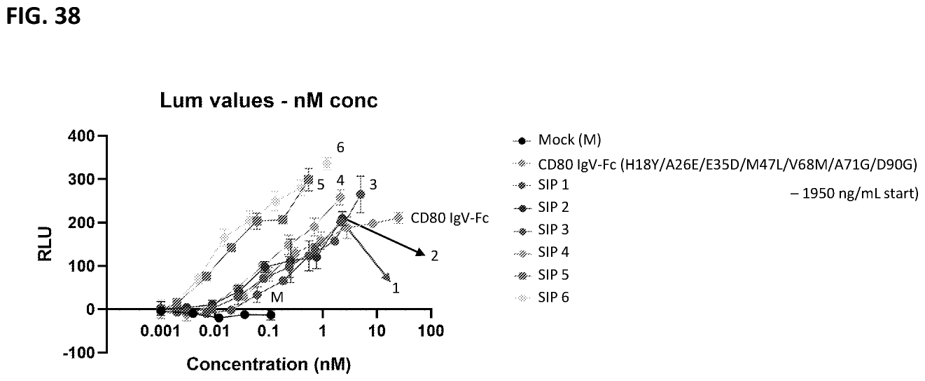

As shown in FIG. 38, exposure to SIP-containing supernatant resulted in increased CD28 costimulation and downstream signal transduction leading to activation of the IL-2 promoter with consequent luciferase induction from the reporter cell line. CD28 costimulation EC50 values for Jurkat- and Pan T-cell-secreted SIPs are presented in Table E42 below. Increased CD28 costimulation (as measured by luciferase activity) and a lower EC50 was observed for the multivalent SIP formats set forth as exemplary SIP 5 and SIP 6.

TABLE E42
CD28 Costimulation EC50 Values for Jurkat- and Pan
T-Cell-Secreted SIPs

| | | EC50 (pM) | EC50 (pM) |
|---|---|---|---|
| | EC50 (pM) | Donor 1 | Donor 2 |
| SIP Variant | Jurkat cells | Pan T-cells | Pan T-cells |
| SIP 1 (Purified) | 197 | 121 | 197 |
| SIP 1 (Supernatant) | 538 | 314 | 538 |
| SIP 2 (Supernatant) | 750 | 282 | 750 |
| SIP 3 (Supernatant) | 961 | 294 | 961 |
| SIP 4 (Supernatant) | 221 | 153 | 221 |
| SIP 5 (Supernatant) | 27 | 73 | 27 |
| SIP 6 (Supernatant) | 21 | 39 | 21 |

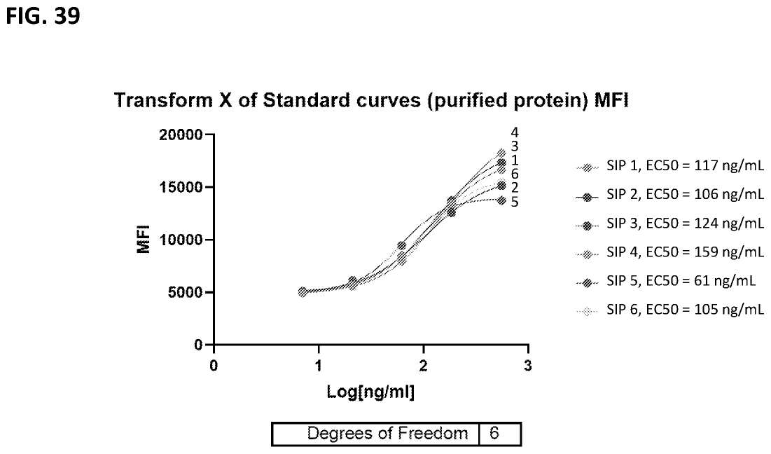

A cell-binding assay was performed to assess the binding of CD80 IgV-Fc SIPs to PD-L1-expressing K562-derived aAPCs. K562/OKT3/PD-L1+ cells were incubated with purified CD80 IgV-Fc SIPs at a starting concentration of 5 μg/mL and at 1:3 serial dilutions thereof. Bound CD80 IgV-Fc SIPs were detected using flow cytometry. SIP-binding standard curves and EC50 values are shown in FIG. 39.

Together, the results are consistent with an observation that T cells engineered to express soluble CD80-IgV SIP will block PD-L1 and PD1 interaction and engage CD28 costimulatory signal in a PDL1I-dependent manner. The results further confirm the increased potency of multivalent formats for inducing CD28-mediated costimulation.

### Example 31

### Assessment of Binding Activity of CD80 ECD-Fc Variants

CD80 ECD-Fc proteins were constructed with CD80 variants identified from the screens described above. Fusion proteins containing the full extracellular domain (ECD) of CD80 set forth in SEQ ID NO:2 in which is contained noted amino acid substitutions were fused to a human IgG1 Fc that has effector activity. As a control, an Fc fusion protein containing the ECD of wild-type CD80 set forth in SEQ ID NO:2 also was generated. The exemplary generated molecules included CD80 (A91G/I118V/T120S/T130A) ECD-Fc, CD80 (S21P/L70Q/D90G/I118V/T120S/T130A) ECD-Fc, CD80 (E88D/K89R/D90K/A91G/F92Y/K93R) ECD-Fc, CD80 (E35D/D46E/M47V/V68M/D90G/K93E) ECD-Fc, and a wildtype CD80 ECD-Fc. The effector Fc, which is set forth in SEQ ID NO:1527 also included the mutation C220S by EU numbering (corresponding to C5S of the Fc) and further contained removal of the C-terminal lysine, K447del by EU numbering (corresponding to deletion of position 232) with reference to wild-type human IgG1 Fc (set forth in SEQ ID NO: 1502).

The CD80-ECD-Fc variants were tested for binding to cell-expressed binding partners substantially as described in Example 7. The EC50 for binding also was determined. The results for binding are provided in Table E43. As shown, when formatted with the full ECD, all tested variant molecules exhibited a very low level of binding to PD-L1 similar to the wild-type CD80 ECD, such that the degree of binding was determined to represent substantially no detectable binding to PD-L1. In contrast, all exemplary molecules exhibited increased binding affinity to CTLA-4 compared to wild-type CD80 and, in some cases, an increase in binding affinity for CD28.

TABLE E43
Flow Binding to CHO cells stably expressing CTLA4
or PD-L1 and Jurkats (CD28)

| | | Binding to Transfected Cells | | | | | |
|---|---|---|---|---|---|---|---|
| | | CHO/ | | Jurkat/ | | CHO/ | |
| | | CTLA4 | | CD28 | | PD-L1 | |
| | | MFI | | MFI | | MFI | |
| | | at | | at | | at | |
| Protein | SEQ | 66.7 | EC50 | 66.7 | EC50 | 66.7 | EC50 |
| Description | ID NO | nM | (nM) | nM | (nM) | nM | (nM) |
| CD80 (A91G/ | 1390 | 24801 | 4.3 | 720 | >200 | 107 | No |
| I118V/ | | | | | | | |
| T120S/T130A) | | | | | nM | | Bind |
| ECD-Fc | | | | | | | |
| CD80 | 1541 | 27021 | 8.8 | 282 | >200 | 103 | No |
| (S21P/L70Q/ | | | | | nM | | Bind |
| D90G/I118V/ | | | | | | | |
| T120S/T130A) | | | | | | | |
| ECD-Fc | | | | | | | |
| CD80 (E88D/ | 1494 | 26384 | 3.6 | 4049 | >200 | 108 | No |
| K89R/D90K/ | | | | | nM | | Bind |
| A91G/F92Y/ | | | | | | | |
| K93R) | | | | | | | |
| ECD-Fc | | | | | | | |
| CD80 (E35D | 307 | 24566 | 10.5 | 343 | >200 | 287 | No |
| /D46E/M47V/ | | | | | nM | | Bind |
| V68M/D90G/ | | | | | | | |
| K93E) | | | | | | | |
| ECD-Fc | | | | | | | |
| Wildtype CD80 | 2 | 25337 | 4.0 | 120 | >200 | 104 | No |
| ECD-Fc | | | | | nM | | Bind |
| CD80 | 299 | 17820 | 3.9 | 5136 | 11.8 | 15065 | 14.5 |
| (H18Y/A26E/ | | | | | | | |
| E35D/M47L/ | | | | | | | |
| V68M/A71G/ | | | | | | | |
| D90G) | | | | | | | |
| IgV-Fc1.1 | | | | | | | |
| Human IgG1 | | | No | | No | | No |
| Isotype | | 69.4 | Bind | 73.2 | Bind | 96.4 | Bind |
| Control | | | | | | | |

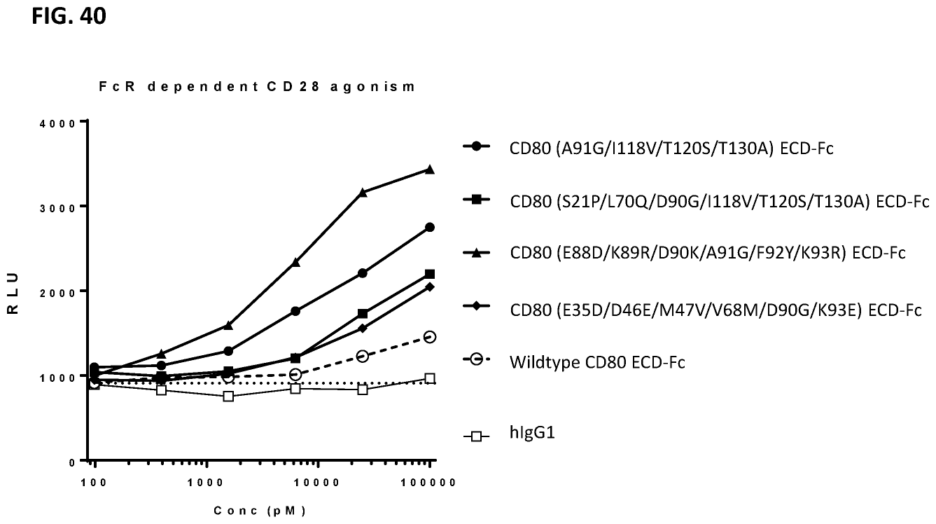

The bioactivity of the generated molecules also was assessed using a Jurkat report assay as described in Examples 9 to assess CD28 costimulation of the molecules. In this assay, the artificial antigen presenting cells (K562/OKT3) did not express PD-L1. The results from the Jurkat reporter assay are provided in Table E44 and FIG. 40. As shown, the generated variants CD80 molecules exhibited an increase in CD28 costimulation in this assay compared to wild-type CD80, which was dose-dependent. This result indicates that these molecules were able to mediate CD28 agonism.

TABLE E44
Jurkat/IL2 + CHO/OKT3 Reporter Assay:
Relative Luciferase Units (RLU)

| | | Relative Luminescence at each | | | | | |
|---|---|---|---|---|---|---|---|
| | SEQ | concentration (RLU) | | | | | |
| | ID | 100000 | 25000 | 6250 | 1563 | 391 | 98 |
| Protein Description | NO | pM | pM | pM | pM | pM | pM |
| CD80 (A91G/I118V/ | 1390 | 2750 | 2209 | 1761 | 1288 | 1119 | 1098 |
| T120S/T130A) | | | | | | | |
| ECD-Fc | | | | | | | |
| CD80 | 1541 | 2197 | 1730 | 1203 | 1049 | 993 | 1042 |
| (S21P/L70Q/D90G/ | | | | | | | |
| I118V/T120S/T130A) | | | | | | | |
| ECD-Fc | | | | | | | |
| CD80 (E88D/K89R/ | 1494 | 3434 | 3162 | 2338 | 1594 | 1258 | 998 |
| D90K/A91G/ | | | | | | | |
| F92Y/K93R) | | | | | | | |
| ECD-Fc | | | | | | | |
| CD80 | 307 | 2046 | 1558 | 1213 | 1022 | 939 | 949 |
| (E35D/D46E/M47V/ | | | | | | | |
| V68M/D90G/K93E) | | | | | | | |
| ECD-Fc | | | | | | | |
| Wildtype CD80 | 2 | 1454 | 1228 | 1012 | 984 | 976 | 915 |
| ECD-Fc | | | | | | | |
| Human IgG1 | | 965 | 835 | 845 | 754 | 828 | 894 |
| Isotype Ctl | | | | | | | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of treating a cancer in a subject, the method comprising:
(a) administering to a subject having a cancer a variant CD80-Fc fusion protein, wherein the variant CD80 fusion protein comprises (1) a variant CD80 extracellular domain or a portion thereof comprising an IgV domain and (2) an Fc domain, wherein the variant CD80 extracellular domain or the portion thereof comprises one or more amino acid substitutions at one or more positions in the sequence of amino acids of the extracellular domain or a portion thereof of an unmodified CD80 polypeptide,
wherein the variant CD80 fusion protein exhibits increased binding to PD-L1 compared to a fusion protein comprising the extracellular domain or portion thereof of the unmodified CD80 for PD-L1; and
(b) administering to the subject a therapeutically effective amount of an anti-cancer agent.

2. The method of claim 1, wherein the anti-cancer agent is an immune checkpoint inhibitor or a chemotherapeutic agent.

3-6. (canceled)

7. The method of claim 1, wherein the anti-cancer agent is an immune checkpoint inhibitor of PD-1 (PD-1 inhibitor).

8. A method of treating a cancer in a subject, the method comprising:
(a) administering to a subject having a cancer a variant CD80-Fc fusion protein, wherein the variant CD80-Fc fusion protein comprises (1) a variant CD80 extracellular domain or a portion thereof comprising an IgV domain and (2) an Fc domain, wherein the variant CD80 extracellular domain or the portion thereof comprises one or more amino acid substitutions at one or more positions in the sequence of amino acids of the extracellular domain or a portion thereof of an unmodified CD80 polypeptide,
wherein the variant CD80 fusion protein exhibits increased binding to PD-L1 compared to a fusion protein comprising the extracellular domain or portion thereof of the unmodified CD80 for PD-L1; and
(b) administering to the subject a therapeutically effective amount of a PD-1 inhibitor, wherein the PD-1 inhibitor disrupts the interaction between Programmed Death-1 (PD-1) and a ligand thereof.

9-12. (canceled)

13. The method of claim 8, wherein the PD-1 inhibitor is an antibody or antigen-binding fragment thereof that specifically binds to PD-1.

14. The method of claim 13, wherein the antibody or antigen-binding portion is selected from nivolumab, pembrolizumab, MEDI0680 (AMP514), PDR001, cemiplimab (REGN2810), pidilizumab (CT011), or an antigen-binding portion thereof.

15. The method of claim 8, wherein the PD-1 inhibitor is pembrolizumab.

16-22. (canceled)

23. The method of claim 8, wherein the variant CD80 fusion protein is administered subcutaneously.

24. The method of claim 8, wherein the variant CD80 fusion protein is administered intravenously.

25-31. (canceled)

32. A method of treating a cancer in a subject, the method comprising intratumorally administering to a subject having a cancer a therapeutically effective amount of a variant CD80-Fc fusion protein, wherein the variant CD80-Fc fusion protein comprises (1) a variant CD80 extracellular domain or a portion thereof comprising an IgV domain and (2) an Fc domain, wherein the variant CD80 extracellular domain or the portion thereof comprises one or more amino acid substitutions at one or more positions in the sequence of amino acids of the extracellular domain or a portion thereof of an unmodified CD80 polypeptide wherein the variant CD80 fusion protein exhibits increased binding to PD-L1 compared to a fusion protein comprising the extracellular domain or portion thereof of the unmodified CD80 for PD-L1.

33-42. (canceled)

43. The methods of claim 1, wherein the variant CD80-Fc fusion protein is administered in an amount of between 1.0 mg/kg to 10 mg/kg, inclusive, once every week (Q1W).

44. A method of treating a cancer in a subject, the method comprising administering to a subject having a cancer a variant CD80-Fc fusion protein in an amount of between about 1.0 mg/kg to 10 mg/kg, inclusive, once every week (Q1W), wherein the variant CD80-Fc fusion protein comprises (1) a variant CD80 extracellular domain or a portion thereof comprising an IgV domain and (2) a an Fc domain, wherein the variant CD80 extracellular domain or the portion thereof comprises one or more amino acid substitutions at one or more positions in the sequence of amino acids of the extracellular domain or a portion thereof of an unmodified CD80 polypeptide, wherein the variant CD80 fusion protein exhibits increased binding to PD-L1 compared to a fusion protein comprising the extracellular domain or portion thereof of the unmodified CD80 for PD-L1.

45. The method of claim 44, wherein the amount of the variant CD80 fusion protein administered Q1W is between about 1 mg/kg and about 3 mg/kg.

46. (canceled)

47. The method of claim 1, wherein the variant CD80-Fc fusion protein is administered in an amount between about 1.0 mg/kg and about 40 mg/kg, inclusive, once every three weeks (Q3W).

48. A method of treating a cancer in a subject, the method comprising administering to a subject having a cancer a variant CD80-Fc fusion protein in an amount of between about 1.0 mg/kg to 40 mg/kg, inclusive, once every three weeks (Q3W), wherein the variant CD80-Fc fusion protein comprises (1) a variant CD80 extracellular domain or a portion thereof comprising an IgV domain and (2) an Fc domain, wherein the variant CD80 extracellular domain or the portion thereof comprises one or more amino acid substitutions at one or more positions in the sequence of amino acids of the extracellular domain or a portion thereof of an unmodified CD80 polypeptide, wherein the variant CD80 fusion protein exhibits increased binding to PD-L1 compared to a fusion protein comprising the extracellular domain or portion thereof of the unmodified CD80 for PD-L1.

49. The method of claim 48, wherein the amount of the variant CD80 fusion protein administered Q3W is between about 3.0 mg/kg and about 10 mg/kg.

50-54. (canceled)

55. The method of claim 1, wherein prior to the administering, selecting a subject for treatment that has a tumor comprising cells surface positive for PD-L1 or CD28 and/or surface negative for a cell surface ligand selected from CD80 or CD86.

56. A method of treating a cancer in a subject, the method comprising administering a variant CD80-Fc fusion protein to a subject selected as having a tumor comprising cells surface negative for a cell surface ligand selected from CD80 or CD86, and/or surface positive for CD28, wherein the variant CD80-Fc fusion protein comprises (1) a variant CD80 extracellular domain or a portion thereof comprising an IgV domain and (1) an Fc domain, said variant CD80 extracellular domain or the portion thereof comprising one or more amino acid substitutions at one or more positions in the sequence of amino acids of the extracellular domain or a portion thereof of an unmodified CD80 polypeptide.

57-58. (canceled)

59. The method of claim 1, wherein the subject has been selected as having a tumor comprising cells surface positive for PD-L1.

60-67. (canceled)

68. The method of claim 1, wherein the one or more amino acid substitutions are selected from among H18Y, A26E, E35D, D46E, D46V, M47I, M47L, M47V, V68M, A71D, A71G, L85M, L85Q or D90G, with reference to numbering of SEQ ID NO:2, or a conservative amino acid substitution thereof.

69. (canceled)

70. The method of claim 1, wherein the one or more amino acid substitutions comprise H18Y/E35D, E35D/D46E, E35D/D46V, E35D/M47I, E35D/M47L, E35D/M47V, E35D/V68M, E35D/L85M, E35D/L85Q, D46E/M47I, D46E/M47L, D46E/M47V, D46V/M47I, D46V/M47L, D46V/M47L, D46E/V68M, D46V/V68M, H18Y/M47I, H18Y/M47L, H18Y/M47V, M47I/V68M, M47L/V68M or M47V/V68M, M47I/E85M, M47L/E85M, M47V/E85M, M47I/E85Q, M47L/E85Q or M47V/E85Q, with reference to numbering of SEQ ID NO:2.

71. The method of claim 1, wherein the one or more amino acid modifications comprise amino acid substitutions E35D/M47L/V68M, E35D/M47V/V68M or E35D/M47I/L70M.

72. The method of claim 1, wherein the one or more amino acid modifications comprise amino acid substitutions E35D/M47V/N48K/V68M/K89N, H18Y/A26E/E35D/M47I/V68M/A71G/D90G, E35D/D46E/M47V/V68M/D90G/K93E, or E35D/D46V/M47I/V68M/L850/E88D.

73-76. (canceled)

77. The method of claim 1, wherein the extracellular domain or portion thereof of the unmodified CD80 comprises (i) the sequence of amino acids set forth in SEQ ID NO:2, (ii) is a portion of (i) comprising an IgV domain.

78-81. (canceled)

82. The method of claim 1, wherein the variant CD80 extracellular domain or the portion thereof comprising the IgV domain comprises the sequence of amino acids 35-135 of SEQ ID NO:2 (SEQ ID NO:76) or 35-141 of SEQ ID NO:2 (SEQ ID NO:150) in which is contained the one or more amino acid substitutions.

83. The method of claim 1, wherein the variant CD80 extracellular domain or the portion thereof comprising the IgV domain is the sequence of amino acids 35-135 of SEQ ID NO:2 (SEQ ID NO:76) or 35-141 of SEQ ID NO:2 (SEQ ID NO:150) in which is contained the one or more amino acid substitutions.

84-85. (canceled)

86. The method of claim 1, wherein the amino acid sequence of the variant CD80 extracellular domain has at least or at least about 85%, sequence identity to the sequence of amino acids 35-135 of SEQ ID NO:2 (SEQ ID NO:76) or 35-141 of SEQ ID NO:2 (SEQ ID NO:150).

87-89. (canceled)

90. The method of claim 1, wherein the Fc region is a variant IgG1 Fc region comprising one or more amino acid substitutions in a wildtype Fc region, said variant Fc region exhibiting one or more effector function that is reduced compared to the wildtype Fc region.

91. The method of claim 90, wherein the Fc region comprises the amino acid substitution N297G, R292C/N297G/V302C, or L234A/L235E/G237A, each wherein the residue is numbered according to the EU index of Kabat.

92. The method of claim 90, wherein the Fc region is set forth in SEQ ID NO: 1508 or SEQ ID NO:1518.

93. The method of claim 90, wherein the variant CD80 extracellular domain or the portion comprising the IgV domain is linked to the Fc region via a linker.

94. The method of claim 93, wherein the linker is GSGGGGS (SEQ ID NO:1522), GS(G₄S)₃ (SEQ ID NO:1243) or GS(G₄S)₅ (SEQ ID NO:1244).

95. The method of claim 1, wherein the variant CD80 fusion protein is a homodimer of two variant CD80-Fc fusion proteins that are the same.

96. The method of claim 1, wherein the cancer is selected from the group consisting of melanoma, bladder cancer, leukemia, lymphoma, myeloma, liver cancer, brain cancer, renal cancer, breast cancer, pancreatic cancer, colorectal cancer, lung cancer, spleen cancer, cancer of the thymus or blood cells, prostate cancer, testicular cancer, ovarian cancer, uterine cancer, gastric carcinoma, a musculoskeletal cancer, a head and neck cancer, a gastrointestinal cancer, a germ cell cancer, or an endocrine and neuroendocrine cancer.

97. The method of claim 1, wherein the cancer is selected from the group consisting of melanoma, non-small cell lung cancer (NSCLC), renal cell carcinoma (RCC), gastric cancer, bladder cancer, diffuse large B-cell lymphoma (DLBCL), Hodgkin's lymphoma, ovarian cancer, head & neck squamous cell cancer (HNSCC), mesothelioma, and triple negative breast cancer (TNBC).

98-223. (canceled)
